(19) United States
(12) Patent Application Publication
(10) Pub. No.: US 2026/0248962 A1
(43) Pub. Date: Aug. 27, 2026

(54) COMPOSITIONS, SYSTEMS, AND METHODS FOR TARGETED TRANSCRIPTIONAL ACTIVATION

(71) Applicant: Tune Therapeutics, Inc., Seattle, WA (US)

(72) Inventors: Veronica GOUGH, Durham, NC (US); Matthew P. GEMBERLING, Durham, NC (US); Jennifer KWON, Durham, NC (US); Dilara SEN, Durham, NC (US); Matthew WOLPERT, Durham, NC (US)

(73) Assignee: Tune Therapeutics, Inc., Seattle, WA (US)

(21) Appl. No.: 18/993,440

(22) PCT Filed: Jul. 12, 2023
(86) PCT No.: PCT/US2023/070086

§ 371 Date: Jan. 10, 2025

Related U.S. Application Data
(60) Provisional application No. 63/388,592, filed on Jul. 12, 2022.
(60) Provisional application No. 63/393,809, filed on Jul. 29, 2022.
(60) Provisional application No. 63/442,761, filed on Feb. 1, 2023.

(51) Int. Cl. A61K 48/00; A61K 38/17; C07K 14/47; C12N 9/22; C12N 15/11; C12N 15/86; C12N 15/88

(52) U.S. Cl. CPC A61K 48/005; A61K 38/1709; C07K 14/4705; C12N 9/226; C12N 15/111; C12N 15/86; C12N 15/88; C07K 2319/09; C07K 2319/40; C07K 2319/71; C07K 2319/80; C07K 2319/81

(57) ABSTRACT

Provided in some aspects are transcriptional activation domains for targeted transcriptional activation. Also provided are multipartite effectors, fusion proteins, and DNA-targeting systems, such as CRISPR/Cas-based DNA-targeting systems, comprising two or more of the transcriptional activation domains. In some aspects, the compositions and methods provided herein facilitate targeted transcriptional activation by targeting the transcriptional activation domains or combinations thereof to a target site, such as a target site for a target gene. In some aspects, also provided are methods and uses related to the provided fusion proteins, effectors or DNA-targeting systems or combinations thereof, for example in connection with therapeutic applications.

### CROSS-REFERENCE TO RELATED APPLICATIONS

This application claims priority from U.S. provisional application No. 63/388,592 filed Jul. 12, 2022, U.S. provisional application No. 63/393,809 filed Jul. 29, 2022, and U.S. provisional application No. 63/442,761 filed Feb. 1, 2023, the contents of which are incorporated by reference in their entireties.

### INCORPORATION BY REFERENCE OF SEQUENCE LISTING

The present application is being filed along with a Sequence Listing in electronic format. The Sequence Listing is provided as a file entitled 224742002340SeqList.xml, created Jul. 12, 2023, which is 605,654 bytes in size. The information in the electronic format of the Sequence Listing is herein incorporated by reference in its entirety.

### FIELD

The present disclosure relates in some aspects to transcriptional activation domains for targeted transcriptional activation. Also provided are multipartite effectors, fusion proteins, and DNA-targeting systems, such as CRISPR/Cas-based DNA-targeting systems, comprising two or more of the transcriptional activation domains. In some aspects, the compositions and methods provided herein facilitate targeted transcriptional activation by targeting the transcriptional activation domains or combinations thereof to a target site, such as a target site for a target gene. In some aspects, also provided are methods and uses related to the provided fusion proteins, effectors or DNA-targeting systems or combinations thereof, for example in connection with therapeutic applications.

### BACKGROUND

Targeted epigenetic modification can be used in aspects such as investigating biology and regulation of gene expression. There is a paucity of existing effector domains for targeted epigenetic modifications, and existing approaches for effector domains may not result in the desired effect in various contexts, and improved effector domains, fusion proteins and DNA-targeting systems are needed. Provided are embodiments that meet such and other needs.

### SUMMARY

Provided herein are fusion proteins comprising transcriptional activation domains for targeted transcriptional activation. Also provided are fusion proteins, effector proteins, such as multipartite effector proteins (including multipartite activators), and DNA-targeting systems, such as CRISPR/Cas-based DNA-targeting systems, that comprise two or more of the transcriptional activation domains. In some aspects, the DNA-targeting systems can include any of the fusion proteins provided herein. In some aspects, the DNA-targeting systems also comprise one or more guide RNAs (gRNAs). Also provided are polynucleotides, vectors, cells, and pluralities and combinations thereof, that encode or comprise the fusion proteins or DNA-targeting systems, components thereof or gRNAs. Also provided are methods and uses related to any compositions, for example, in modulating the expression of a target locus, and/or in the treatment or therapy of diseases or disorders.

Provided herein are fusion proteins comprising two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. Provided herein are fusion proteins comprising two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2. In some of any embodiments, h.the transcriptional activation domain increases transcription of an endogenous locus when recruited to a target site at the endogenous locus. In some of any embodiments, the fusion proteins also include a DNA-targeting domain or a component thereof.

Provided herein are fusion proteins comprising a DNA-targeting domain or a component thereof, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. Provided herein are fusion proteins comprising a DNA-targeting domain or a component thereof, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2. In some of any embodiments, the transcriptional activation domain increases transcription of an endogenous locus when recruited to a target site at the endogenous locus.

In some of any embodiments, the DNA-targeting domain comprises a Cas-gRNA combination comprising a Cas protein or a variant thereof, and at least one gRNA that binds to the target site at the endogenous locus, and the component thereof fused to the two or more transcriptional activation domains is the Cas protein or a variant thereof.

In some of any embodiments, the DNA-targeting domain comprises a zinc finger protein (ZFP), a transcription activator-like effector (TALE), a meganuclease, a homing endonuclease, or an I-SceI enzyme or a variant thereof that binds to the target site at the endogenous locus.

Provided herein are fusion proteins comprising a Cas protein or a variant thereof, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. Provided herein are fusion proteins comprising a Cas protein or a variant thereof, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2. In some of any embodiments, the transcriptional activation domain increases transcription of an endogenous locus when recruited to a target site at the endogenous locus.

In some of any embodiments, the Cas protein or a variant thereof is capable of complexing with at least one gRNA.

Provided herein are fusion proteins comprising a zinc finger protein (ZFP), a transcription activator-like effector (TALE), a meganuclease, a homing endonuclease, or an I-SceI enzyme or a variant thereof, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. Provided herein are fusion proteins comprising a zinc finger protein (ZFP), a transcription activator-like effector (TALE), a meganuclease, a homing endonuclease, or an I-SceI enzyme or a variant thereof, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2. In some of any embodiments, the transcriptional activation domain increases transcription of an endogenous locus when recruited to a target site at the endogenous locus.

In some of any embodiments, the variant thereof comprises a catalytically inactive variant.

In some of any embodiments, the Cas protein or a variant thereof is a Cas9 or a variant thereof. In some of any embodiments, the Cas protein or a variant thereof protein is a deactivated Cas9 (dCas9).

In some of any embodiments, the Cas protein or a variant thereof is a Staphylococcus aureus Cas9 (SaCas9) or a variant thereof. In some of any embodiments, the Cas protein or a variant thereof is a Staphylococcus aureus dCas9 (dSaCas9) that comprises at least one amino acid mutation selected from D10A and N580A, with reference to numbering of positions of SEQ ID NO:3. In some of any embodiments, the Cas protein or a variant thereof comprises the sequence set forth in SEQ ID NO:2, and an amino acid sequence that has at least 90%, 91%, 92%, 93%, 94%, 95%, 96%, 97%, 98%, and 99% sequence identity thereto.

In some of any embodiments, the Cas9 or variant thereof is a Streptococcus pyogenes Cas9 (SpCas9) or a variant thereof. In some of any embodiments, the Cas protein or a variant thereof is a Streptococcus pyogenes dCas9 (dSpCas9) that comprises at least one amino acid mutation selected from D10A and H840A, with reference to numbering of positions of SEQ ID NO:7. In some of any embodiments, the Cas protein or a variant thereof comprises the sequence set forth in SEQ ID NO:6, and an amino acid sequence that has at least 90%, 91%, 92%, 93%, 94%, 95%, 96%, 97%, 98%, and 99% sequence identity thereto.

In some of any embodiments, the Cas protein or a variant thereof is a split variant Cas protein, wherein the split variant Cas protein comprises a first polypeptide comprising an N-terminal fragment of the variant Cas protein and an N-terminal Intein, and a second polypeptide comprising a C-terminal fragment of the variant Cas protein and a C-terminal Intein. In some of any embodiments, when the first polypeptide and the second polypeptide of the split variant Cas protein are present in proximity or present in the same cell, the N-terminal Intein and C-terminal Intein self-excise and ligate the N-terminal fragment and the C-terminal fragment of the variant Cas protein to form a full-length variant Cas protein. In some of any embodiments, the N-terminal Intein comprises an N-terminal Npu Intein, or the sequence set forth in SEQ ID NO:88, or an amino acid sequence that has at least 90%, 91%, 92%, 93%, 94%, 95%, 96%, 97%, 98%, or 99% sequence identity thereto, or a portion of any of the foregoing. In some of any embodiments, N-terminal fragment of the variant Cas protein comprises: the N-terminal fragment of variant SpCas9 from the N-terminal end up to position 573 of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or the sequence set forth in SEQ ID NO:86, or an amino acid sequence that has at least 90%, 91%, 92%, 93%, 94%, 95%, 96%, 97%, 98%, or 99% sequence identity thereto, or a portion of any of the foregoing. In some of any embodiments, the C-terminal Intein comprises a C-terminal Npu Intein, or the sequence set forth in SEQ ID NO:92, or an amino acid sequence that has at least 90%, 91%, 92%, 93%, 94%, 95%, 96%, 97%, 98%, or 99% sequence identity thereto, or a portion of any of the foregoing. In some of any embodiments, C-terminal fragment of the variant Cas protein comprises: the C-terminal fragment of variant SpCas9 from position 574 to the C-terminal end of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or the sequence set forth in SEQ ID NO:94, or an amino acid sequence that has at least 90%, 91%, 92%, 93%, 94%, 95%, 96%, 97%, 98%, or 99% sequence identity thereto, or a portion of any of the foregoing.

In some of any embodiments, the Cas protein or a variant thereof is a Cpf1 or a variant thereof. In some of any embodiments, the Cas protein or a variant thereof is a variant Cpf1 that that is a deactivated Cpf1 (dCpf1). In some of any embodiments, the variant comprises a catalytically inactive nuclease variant.

In some of any embodiments, the transcriptional activation domain of NCOA3 comprises: (i) the sequence set forth in SEQ ID NO:40; (ii) a contiguous portion of SEQ ID NO:40 of at least 20 amino acids; (iii) the sequence set forth in SEQ ID NO:27; (iv) a contiguous portion of SEQ ID NO:27 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of NCOA3 comprises the sequence set forth in SEQ ID NO:133 or an amino acid sequence that has at least 90%, 91%, 92%, 93%, 94%, 95%, 96%, 97%, 98%, or 99% sequence identity to SEQ ID NO:133. In some of any embodiments, the transcriptional activation domain of NCOA3 comprises the sequence set forth in SEQ ID NO:133.

In some of any embodiments, the transcriptional activation domain of ENL comprises: (i) the sequence set forth in SEQ ID NO:36; (ii) a contiguous portion of SEQ ID NO:36 of at least 20 amino acids; (iii) the sequence set forth in SEQ ID NO:23; (iv) a contiguous portion of SEQ ID NO:23 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of ENL comprises the sequence set forth in SEQ ID NO:131 or an amino acid sequence that has at least 90%, 91%, 92%, 93%, 94%, 95%, 96%, 97%, 98%, or 99% sequence identity to any of the foregoing. In some of any embodiments, the transcriptional activation domain of ENL comprises the sequence set forth in SEQ ID NO:131.

In some of any embodiments, the transcriptional activation domain of FOXO3 comprises: (i) the sequence set forth in SEQ ID NO:37; (ii) a contiguous portion of SEQ ID NO:37 of at least 20 amino acids; (iii) the sequence set forth in SEQ ID NO:24; (iv) a contiguous portion of SEQ ID NO:24 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of FOXO3 comprises the sequence set forth in SEQ ID NO:132 or an amino acid sequence that has at least 90%, 91%, 92%, 93%, 94%, 95%, 96%, 97%, 98%, or 99% sequence identity to any of the foregoing. In some of any embodiments, the transcriptional activation domain of FOXO3 comprises the sequence set forth in SEQ ID NO:132.

In some of any embodiments, the transcriptional activation domain of PYGO1 comprises: (i) the sequence set forth in SEQ ID NO:42; (ii) a contiguous portion of SEQ ID NO:42 of at least 20 amino acids; (iii) the sequence set forth in SEQ ID NO:29; (iv) a contiguous portion of SEQ ID NO:29 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of PYGO1 comprises the sequence set forth in SEQ ID NO:130 or an amino acid sequence that has at least 90%, 91%, 92%, 93%, 94%, 95%, 96%, 97%, 98%, or 99% sequence identity to any of the foregoing. In some of any embodiments, the transcriptional activation domain of PYGO1 comprises the sequence set forth in SEQ ID NO:130.

In some of any embodiments, the transcriptional activation domain of HSH2D comprises: (i) the sequence set forth in SEQ ID NO:38; (ii) a contiguous portion of SEQ ID NO:38 of at least 20 amino acids; (iii) the sequence set forth in SEQ ID NO:25; (iv) a contiguous portion of SEQ ID NO:25 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of HSH2D comprises the sequence set forth in SEQ ID NO:134 or an amino acid sequence that has at least 90%, 91%, 92%, 93%, 94%, 95%, 96%, 97%, 98%, or 99% sequence identity to any of the foregoing. In some of any embodiments, the transcriptional activation domain of HSH2D comprises the sequence set forth in SEQ ID NO:134.

In some of any embodiments, the transcriptional activation domain of NCOA2 comprises: (i) the sequence set forth in SEQ ID NO:39; (ii) a contiguous portion of SEQ ID NO:39 of at least 20 amino acids; (iii) the sequence set forth in SEQ ID NO:26; (iv) a contiguous portion of SEQ ID NO:26 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of NCOA2 comprises the sequence set forth in SEQ ID NO:135 or an amino acid sequence that has at least 90%, 91%, 92%, 93%, 94%, 95%, 96%, 97%, 98%, or 99% sequence identity to any of the foregoing. In some of any embodiments, the transcriptional activation domain of NCOA2 comprises the sequence set forth in SEQ ID NO:135.

In some of any embodiments, the transcriptional activation domain of NOTCH2 comprises: (i) the sequence set forth in SEQ ID NO:46; (ii) a contiguous portion of SEQ ID NO:46 of at least 20 amino acids; (iii) the sequence set forth in SEQ ID NO:33; (iv) a contiguous portion of SEQ ID NO:33 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of NOTCH2 comprises the sequence set forth in SEQ ID NO:136 or an amino acid sequence that has at least 90%, 91%, 92%, 93%, 94%, 95%, 96%, 97%, 98%, or 99% sequence identity to any of the foregoing. In some of any embodiments, the transcriptional activation domain of NOTCH2 comprises the sequence set forth in SEQ ID NO:136.

In some of any embodiments, the transcriptional activation domain of NOTCH2 comprises: (i) the sequence set forth in SEQ ID NO:46 or 390; (ii) a contiguous portion of SEQ ID NO:46 or 390 of at least 20 amino acids; (iii) the sequence set forth in SEQ ID NO:33 or 381; (iv) a contiguous portion of SEQ ID NO:33 or 381 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of NOTCH2 comprises the sequence set forth in SEQ ID NO:136 or an amino acid sequence that has at least 90%, 91%, 92%, 93%, 94%, 95%, 96%, 97%, 98%, or 99% sequence identity to any of the foregoing. In some of any embodiments, the transcriptional activation domain of NOTCH2 comprises the sequence set forth in SEQ ID NO:136.

In some of any embodiments, the transcriptional activation domain of DPOLA comprises: (i) the sequence set forth in SEQ ID NO:35; (ii) a contiguous portion of SEQ ID NO:35 of at least 20 amino acids; (iii) the sequence set forth in SEQ ID NO:22; (iv) a contiguous portion of SEQ ID NO:22 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of DPOLA comprises the sequence set forth in SEQ ID NO:176 or an amino acid sequence that has at least 90%, 91%, 92%, 93%, 94%, 95%, 96%, 97%, 98%, or 99% sequence identity to any of the foregoing. In some of any embodiments, the transcriptional activation domain of DPOLA comprises the sequence set forth in SEQ ID NO:176.

In some of any embodiments, the transcriptional activation domain of PSA1 comprises: (i) the sequence set forth in SEQ ID NO:41; (ii) a contiguous portion of SEQ ID NO:41 of at least 20 amino acids; (iii) the sequence set forth in SEQ ID NO:28; (iv) a contiguous portion of SEQ ID NO:28 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of PSA1 comprises the sequence set forth in SEQ ID NO:177 or an amino acid sequence that has at least 90%, 91%, 92%, 93%, 94%, 95%, 96%, 97%, 98%, or 99% sequence identity to any of the foregoing. In some of any embodiments, the transcriptional activation domain of PSA1 comprises the sequence set forth in SEQ ID NO:177.

In some of any embodiments, the transcriptional activation domain of RBM39 comprises: (i) the sequence set forth in SEQ ID NO:43; (ii) a contiguous portion of SEQ ID NO:43 of at least 20 amino acids; (iii) the sequence set forth in SEQ ID NO:30; (iv) a contiguous portion of SEQ ID NO:30 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of RBM39 comprises the sequence set forth in SEQ ID NO:178 or an amino acid sequence that has at least 90%, 91%, 92%, 93%, 94%, 95%, 96%, 97%, 98%, or 99% sequence identity to any of the foregoing. In some of any embodiments, the transcriptional activation domain of RBM39 comprises the sequence set forth in SEQ ID NO:178.

In some of any embodiments, the transcriptional activation domain of HERC2 comprises: (i) the sequence set forth in SEQ ID NO:44; (ii) a contiguous portion of SEQ ID NO:44 of at least 20 amino acids; (iii) the sequence set forth in SEQ ID NO:31; (iv) a contiguous portion of SEQ ID NO:31 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of HERC2 comprises the sequence set forth in SEQ ID NO:179 or an amino acid sequence that has at least 90%, 91%, 92%, 93%, 94%, 95%, 96%, 97%, 98%, or 99% sequence identity to any of the foregoing. In some of any embodiments, the transcriptional activation domain of HERC2 comprises the sequence set forth in SEQ ID NO:179.

In some of any of the embodiments, the transcriptional activation domain of ZNF473 comprises: (i) the sequence set forth in SEQ ID NO:387; (ii) a contiguous portion of SEQ ID NO:387 of at least 20 amino acids; (iii) the sequence set forth in SEQ ID NO:378; (iv) a contiguous portion of SEQ ID NO:378 of at least 20 amino acids; (v) an amino acid sequence that has at least 90%, 91%, 92%, 93%, 94%, 95%, 96%, 97%, 98%, or 99% sequence identity to any of the foregoing.

In some of any of the embodiments, the transcriptional activation domain of ANM2 comprises: (i) the sequence set forth in SEQ ID NO:388; (ii) a contiguous portion of SEQ ID NO:388 of at least 20 amino acids; (iii) the sequence set forth in SEQ ID NO:379; (iv) a contiguous portion of SEQ ID NO:379 of at least 20 amino acids; (v) an amino acid sequence that has at least 90%, 91%, 92%, 93%, 94%, 95%, 96%, 97%, 98%, or 99% sequence identity to any of the foregoing.

In some of any of the embodiments, the transcriptional activation domain of KIBRA comprises: (i) the sequence set forth in SEQ ID NO:389; (ii) a contiguous portion of SEQ ID NO:389 of at least 20 amino acids; (iii) the sequence set forth in SEQ ID NO:380; (iv) a contiguous portion of SEQ ID NO:380 of at least 20 amino acids; (v) an amino acid sequence that has at least 90%, 91%, 92%, 93%, 94%, 95%, 96%, 97%, 98%, or 99% sequence identity to any of the foregoing.

In some of any of the embodiments, the transcriptional activation domain of IKKA comprises: (i) the sequence set forth in SEQ ID NO:391; (ii) a contiguous portion of SEQ ID NO:391 of at least 20 amino acids; (iii) the sequence set forth in SEQ ID NO:382; (iv) a contiguous portion of SEQ ID NO:382 of at least 20 amino acids; (v) an amino acid sequence that has at least 90%, 91%, 92%, 93%, 94%, 95%, 96%, 97%, 98%, or 99% sequence identity to any of the foregoing.

In some of any of the embodiments, the transcriptional activation domain of APBB1 comprises: (i) the sequence set forth in SEQ ID NO:392; (ii) a contiguous portion of SEQ ID NO:392 of at least 20 amino acids; (iii) the sequence set forth in SEQ ID NO:383; (iv) a contiguous portion of SEQ ID NO:383 of at least 20 amino acids; (v) an amino acid sequence that has at least 90%, 91%, 92%, 93%, 94%, 95%, 96%, 97%, 98%, or 99% sequence identity to any of the foregoing.

In some of any of the embodiments, the transcriptional activation domain of SMN2 comprises: (i) the sequence set forth in SEQ ID NO:393; (ii) a contiguous portion of SEQ ID NO:393 of at least 20 amino acids; (iii) the sequence set forth in SEQ ID NO:384; (iv) a contiguous portion of SEQ ID NO:384 of at least 20 amino acids; (v) an amino acid sequence that has at least 90%, 91%, 92%, 93%, 94%, 95%, 96%, 97%, 98%, or 99% sequence identity to any of the foregoing.

In some of any of the embodiments, the transcriptional activation domain of SERTAD2 comprises: (i) the sequence set forth in SEQ ID NO:394; (ii) a contiguous portion of SEQ ID NO:394 of at least 20 amino acids; (iii) the sequence set forth in SEQ ID NO:385; (iv) a contiguous portion of SEQ ID NO:385 of at least 20 amino acids; (v) an amino acid sequence that has at least 90%, 91%, 92%, 93%, 94%, 95%, 96%, 97%, 98%, or 99% sequence identity to any of the foregoing.

In some of any of the embodiments, the transcriptional activation domain of MYBA comprises: (i) the sequence set forth in SEQ ID NO:395; (ii) a contiguous portion of SEQ ID NO:395 of at least 20 amino acids; (iii) the sequence set forth in SEQ ID NO:386; (iv) a contiguous portion of SEQ ID NO:386 of at least 20 amino acids; (v) an amino acid sequence that has at least 90%, 91%, 92%, 93%, 94%, 95%, 96%, 97%, 98%, or 99% sequence identity to any of the foregoing.

In some of any embodiments, the transcriptional activation domain is at least at or about 30, 40, 50, 60, or 70 amino acids in length. In some of any embodiments, the transcriptional activation domain is at least at or about 40 amino acids in length. In some of any embodiments, the transcriptional activation domain is at least at or about 50 amino acids in length. In some of any embodiments, the transcriptional activation domain is at least at or about 60 amino acids in length. In some of any embodiments, the transcriptional activation domain is at least at or about 70 amino acids in length.

In some of any embodiments, the transcriptional activation domain is at or about 120, 110, 100, 90, 80, 70, 60, 50, or 40 amino acids or less in length. In some of any embodiments, the transcriptional activation domain is 70 amino acids or less in length. In some of any embodiments, the transcriptional activation domain is 60 amino acids or less in length. In some of any embodiments, the transcriptional activation domain is 50 amino acids or less in length.

In some of any embodiments, the transcriptional activation domain is between at or about 40 and at or about 120, at or about 40 and at or about 110, at or about 40 and at or about 100, at or about 40 and at or about 90, at or about 40 and at or about 80, at or about 40 and at or about 70, at or about 40 and at or about 60, or at or about 40 and at or about 50 in length.

In some of any embodiments, the two or more transcriptional activation domains is two transcriptional activation domains.

In some of any embodiments, the fusion protein comprises: a transcriptional activation domain of PYGO1 and a transcriptional activation domain of NCOA3; a transcriptional activation domain of NOTCH2 and a transcriptional activation domain of NCOA3; a transcriptional activation domain of NCOA3 and a transcriptional activation domain of NCOA3; a transcriptional activation domain of HSH2D and a transcriptional activation domain of NCOA3; a transcriptional activation domain of FOXO3 and a transcriptional activation domain of NCOA3; a transcriptional activation domain of NCOA2 and a transcriptional activation domain of NCOA3; a transcriptional activation domain of ENL and a transcriptional activation domain of NCOA3; a transcriptional activation domain of PYGO1 and a transcriptional activation domain of FOXO3; a transcriptional activation domain of NOTCH2 and a transcriptional activation domain of FOXO3; a transcriptional activation domain of NCOA3 and a transcriptional activation domain of FOXO3; a transcriptional activation domain of HSH2D and a transcriptional activation domain of FOXO3; a transcriptional activation domain of FOXO3 and a transcriptional activation domain of FOXO3; a transcriptional activation domain of NCOA2 and a transcriptional activation domain of FOXO3; or a transcriptional activation domain of ENL and a transcriptional activation domain of FOXO3. In some of any of the embodiments, the fusion protein comprises: a transcriptional activation domain of MYBA and a transcriptional activation domain of FOXO3. In some of any of the embodiments, the fusion protein comprises: a transcriptional activation domain of SERTAD2 and a transcriptional activation domain of NCOA2.

In some of any embodiments, the fusion protein comprises, in N-terminus to C-terminus order: a transcriptional activation domain of PYGO1, a linker, and a transcriptional activation domain of NCOA3; a transcriptional activation domain of NOTCH2, a linker, and a transcriptional activation domain of NCOA3; a transcriptional activation domain of NCOA3, a linker, and a transcriptional activation domain of NCOA3; a transcriptional activation domain of HSH2D, a linker, and a transcriptional activation domain of NCOA3; a transcriptional activation domain of FOXO3, a linker, and a transcriptional activation domain of NCOA3; a transcriptional activation domain of NCOA2, a linker, and a transcriptional activation domain of NCOA3; a transcriptional activation domain of ENL, a linker, and a transcriptional activation domain of NCOA3; a transcriptional activation domain of PYGO1, a linker, and a transcriptional activation domain of FOXO3; a transcriptional activation domain of NOTCH2, a linker, and a transcriptional activation domain of FOXO3; a transcriptional activation domain of NCOA3, a linker, and a transcriptional activation domain of FOXO3; a transcriptional activation domain of HSH2D, a linker, and a transcriptional activation domain of FOXO3; a transcriptional activation domain of FOXO3, a linker, and a transcriptional activation domain of FOXO3; a transcriptional activation domain of NCOA2, a linker, and a transcriptional activation domain of FOXO3; or a transcriptional activation domain of ENL, a linker, and a transcriptional activation domain of FOXO3.

In some of any embodiments, the fusion protein comprises the sequence set forth in any one of SEQ ID NOS:140-153, or an amino acid sequence that has at least 90%, 91%, 92%, 93%, 94%, 95%, 96%, 97%, 98%, or 99% sequence identity to any of the foregoing. In some of any embodiments, the fusion protein comprises the sequence set forth in SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, or SEQ ID NO:153.

In some of any embodiments, the fusion protein comprises, in N-terminus to C-terminus order: a dCas9, a transcriptional activation domain of PYGO1 and a transcriptional activation domain of NCOA3; a dCas9, a transcriptional activation domain of NOTCH2 and a transcriptional activation domain of NCOA3; a dCas9, a transcriptional activation domain of NCOA3 and a transcriptional activation domain of NCOA3; a dCas9, a transcriptional activation domain of HSH2D and a transcriptional activation domain of NCOA3; a dCas9, a transcriptional activation domain of FOXO3 and a transcriptional activation domain of NCOA3; a dCas9, a transcriptional activation domain of NCOA2 and a transcriptional activation domain of NCOA3; a dCas9, a transcriptional activation domain of ENL and a transcriptional activation domain of NCOA3; a dCas9, a transcriptional activation domain of PYGO1 and a transcriptional activation domain of FOXO3; a dCas9, a transcriptional activation domain of NOTCH2 and a transcriptional activation domain of FOXO3; a dCas9, a transcriptional activation domain of NCOA3 and a transcriptional activation domain of FOXO3; a dCas9, a transcriptional activation domain of HSH2D and a transcriptional activation domain of FOXO3; a dCas9, a transcriptional activation domain of FOXO3 and a transcriptional activation domain of FOXO3; a dCas9, a transcriptional activation domain of NCOA2 and a transcriptional activation domain of FOXO3; or a dCas9, a transcriptional activation domain of ENL and a transcriptional activation domain of FOXO3. In some of any embodiments, the fusion protein comprises, in N-terminus to C-terminus order: a transcriptional activation domain of PYGO1, a transcriptional activation domain of NCOA3, and a dCas9; a transcriptional activation domain of NOTCH2, a transcriptional activation domain of NCOA3, and a dCas9; a transcriptional activation domain of NCOA3, a transcriptional activation domain of NCOA3, and a dCas9; a transcriptional activation domain of HSH2D, a transcriptional activation domain of NCOA3, and a dCas9; a transcriptional activation domain of FOXO3, a transcriptional activation domain of NCOA3, and a dCas9; a transcriptional activation domain of NCOA2, a transcriptional activation domain of NCOA3, and a dCas9; a transcriptional activation domain of ENL, a transcriptional activation domain of NCOA3, and a dCas9; a transcriptional activation domain of PYGO1, a transcriptional activation domain of FOXO3, and a dCas9; a transcriptional activation domain of NOTCH2, a transcriptional activation domain of FOXO3, and a dCas9; a transcriptional activation domain of NCOA3, a transcriptional activation domain of FOXO3, and a dCas9; a transcriptional activation domain of HSH2D, a transcriptional activation domain of FOXO3, and a dCas9; a transcriptional activation domain of FOXO3, a transcriptional activation domain of FOXO3, and a dCas9; a transcriptional activation domain of NCOA2, a transcriptional activation domain of FOXO3, and a dCas9; or a transcriptional activation domain of ENL, a transcriptional activation domain of FOXO3, and a dCas9.

In some of any embodiments, the two or more transcriptional activation domains is three transcriptional activation domains.

In some of any embodiments, the fusion protein comprises: a transcriptional activation domain of PYGO1, a transcriptional activation domain of FOXO3, and a transcriptional activation domain of NCOA3; a transcriptional activation domain of NOTCH2, a transcriptional activation domain of FOXO3, and a transcriptional activation domain of NCOA3; a transcriptional activation domain of NCOA3, a transcriptional activation domain of FOXO3, and a transcriptional activation domain of NCOA3; a transcriptional activation domain of HSH2D, a transcriptional activation domain of FOXO3, and a transcriptional activation domain of NCOA3; a transcriptional activation domain of FOXO3, a transcriptional activation domain of FOXO3, and a transcriptional activation domain of NCOA3; a transcriptional activation domain of NCOA2, a transcriptional activation domain of FOXO3, and a transcriptional activation domain of NCOA3; or a transcriptional activation domain of ENL, a transcriptional activation domain of FOXO3, and a transcriptional activation domain of NCOA3. In some of any embodiments, the fusion protein comprises, in N-terminus to C-terminus order: a transcriptional activation domain of PYGO1, a linker, a transcriptional activation domain of FOXO3, a linker, and a transcriptional activation domain of NCOA3; a transcriptional activation domain of NOTCH2, a linker, a transcriptional activation domain of FOXO3, a linker, and a transcriptional activation domain of NCOA3; a transcriptional activation domain of NCOA3, a linker, a transcriptional activation domain of FOXO3, a linker, and a transcriptional activation domain of NCOA3; a transcriptional activation domain of HSH2D, a linker, a transcriptional activation domain of FOXO3, a linker, and a transcriptional activation domain of NCOA3; a transcriptional activation domain of FOXO3, a linker, a transcriptional activation domain of FOXO3, a linker, and a transcriptional activation domain of NCOA3; a transcriptional activation domain of NCOA2, a linker, a transcriptional activation domain of FOXO3, a linker, and a transcriptional activation domain of NCOA3; or a transcriptional activation domain of ENL, a linker, a transcriptional activation domain of FOXO3, a linker, and a transcriptional activation domain of NCOA3.

In some of any embodiments, the fusion protein comprises the sequence set forth in any one of SEQ ID NOS:154-160 or SEQ ID NO: 377 or an amino acid sequence that has at least 90%, 91%, 92%, 93%, 94%, 95%, 96%, 97%, 98%, or 99% sequence identity to any of the foregoing.

In some of any embodiments, the fusion protein comprises the sequence set forth in SEQ ID NO:154, SEQ ID NO:155, SEQ ID NO:156, SEQ ID NO:157, SEQ ID NO:158, SEQ ID NO:159, or SEQ ID NO:160 or SEQ ID NO: 377.

In some of any embodiments, the fusion protein comprises a transcriptional activation domain of PYGO1, a linker, a transcriptional activation domain of FOXO3, a linker, and a transcriptional activation domain of NCOA3. In some of any embodiments, the fusion protein comprises the sequence set forth in SEQ ID NO:154.

In some of any embodiments, the fusion protein comprises a transcriptional activation domain of NCOA3, a linker, a transcriptional activation domain of FOXO3, a linker, and a transcriptional activation domain of NCOA3. In some of any embodiments, the fusion protein comprises the sequence set forth in SEQ ID NO:156.

In some of any embodiments, the fusion protein comprises a transcriptional activation domain of FOXO3, a linker, a transcriptional activation domain of FOXO3, a linker, and a transcriptional activation domain of NCOA3. In some of any embodiments, the fusion protein comprises the sequence set forth in SEQ ID NO:158.

In some of any embodiments, the fusion protein comprises a transcriptional activation domain of NCOA2, a linker, a transcriptional activation domain of FOXO3, a linker, and a transcriptional activation domain of NCOA3. In some of any embodiments, the fusion protein comprises the sequence set forth in SEQ ID NO:159.

In some embodiments, the multipartite activator comprises domains from NCOA3, FOXO3, and FOX03, respectively. In some embodiments, the multipartite activator comprises SEQ ID NO:377, or an amino acid sequence that has at least 90%, 91%, 92%, 93%, 94%, 95%, 96%, 97%, 98%, or 99% sequence identity to SEQ ID NO:377. In some embodiments, the multipartite activator is set forth in SEQ ID NO:377.

In some of any embodiments, the fusion protein comprises, in N-terminus to C-terminus order: a dCas9, a linker, a transcriptional activation domain of PYGO1, a linker, a transcriptional activation domain of FOXO3, a linker, and a transcriptional activation domain of NCOA3; a dCas9, a linker, a transcriptional activation domain of NOTCH2, a linker, a transcriptional activation domain of FOXO3, a linker, and a transcriptional activation domain of NCOA3; a dCas9, a linker, a transcriptional activation domain of NCOA3, a linker, a transcriptional activation domain of FOXO3, a linker, and a transcriptional activation domain of NCOA3; a dCas9, a linker, a transcriptional activation domain of HSH2D, a linker, a transcriptional activation domain of FOXO3, a linker, and a transcriptional activation domain of NCOA3; a dCas9, a linker, a transcriptional activation domain of FOXO3, a linker, a transcriptional activation domain of FOXO3, a linker, and a transcriptional activation domain of NCOA3; a dCas9, a linker, a transcriptional activation domain of NCOA2, a linker, a transcriptional activation domain of FOXO3, a linker, and a transcriptional activation domain of NCOA3; or a dCas9, a linker, a transcriptional activation domain of ENL, a linker, a transcriptional activation domain of FOXO3, a linker, and a transcriptional activation domain of NCOA3.

In some of any embodiments, the fusion protein comprises, in N-terminus to C-terminus order: a transcriptional activation domain of PYGO1, a linker, a transcriptional activation domain of FOXO3, a linker, a transcriptional activation domain of NCOA3, a linker, and a dCas9; a transcriptional activation domain of NOTCH2, a linker, a transcriptional activation domain of FOXO3, a linker, a transcriptional activation domain of NCOA3, a linker, and a dCas9; a transcriptional activation domain of NCOA3, a linker, a transcriptional activation domain of FOXO3, a linker, a transcriptional activation domain of NCOA3, a linker, and a dCas9; a transcriptional activation domain of HSH2D, a linker, a transcriptional activation domain of FOXO3, a linker, a transcriptional activation domain of NCOA3, a linker, and a dCas9; a transcriptional activation domain of FOXO3, a linker, a transcriptional activation domain of FOXO3, a linker, a transcriptional activation domain of NCOA3, a linker, and a dCas9; a transcriptional activation domain of NCOA2, a linker, a transcriptional activation domain of FOXO3, a linker, a transcriptional activation domain of NCOA3, a linker, and a dCas9; or a transcriptional activation domain of ENL, a linker, a transcriptional activation domain of FOXO3, a linker, a transcriptional activation domain of NCOA3, a linker, and a dCas9.

In some of any embodiments, the two or more transcriptional activation domains comprises four transcriptional activation domains. In some of any embodiments, the two or more transcriptional activation domains comprises five transcriptional activation domains.

In some of any embodiments, the fusion proteins also include one or more linkers. In some of any embodiments, a linker of one or more linkers is positioned between the two or more transcriptional activation domains and/or positioned between the polypeptide component of the DNA-targeting domain and one of the two or more transcriptional activation domains. In some of any embodiments, the linker is a polypeptide linker. In some of any embodiments, the polypeptide linker comprises a sequence selected from among SEQ ID NOS:62-67, 96, and 137-139.

In some of any embodiments, the fusion proteins also include one or more nuclear localization signals (NLSs). In some of any embodiments, the one or more NLSs comprises two or more NLSs. In some of any embodiments, a NLS of one or more NLSs is positioned between the two or more transcriptional activation domains. In some of any embodiments, a NLS of the one or more NLSs is positioned between the polypeptide component of the DNA-targeting domain and one of the two or more transcriptional activation domains. In some of any embodiments, the one or more NLSs comprises a sequence selected from among SEQ ID NOS:69-84.

In some of any embodiments, the fusion protein comprises the sequence set forth in any one of SEQ ID NOS:181-187, or an amino acid sequence that has at least 90%, 91%, 92%, 93%, 94%, 95%, 96%, 97%, 98%, or 99% sequence identity to any of the foregoing. In some of any embodiments, the fusion protein comprises the sequence set forth in SEQ ID NO:181, SEQ ID NO:182, SEQ ID NO:183, SEQ ID NO:184, SEQ ID NO:185, SEQ ID NO:186, or SEQ ID NO:187.

In some of any embodiments, the fusion proteins also include a tag. In some of any embodiments, the tag comprises an epitope tag or a split protein tag. In some of any embodiments, the tag is selected from among SEQ ID NOS:61, 88, 92, and 167.

In some of any embodiments, the fusion protein comprises the sequence set forth in any one of SEQ ID NOS:272-278, or an amino acid sequence that has at least 90%, 91%, 92%, 93%, 94%, 95%, 96%, 97%, 98%, or 99% sequence identity to any of the foregoing. In some of any embodiments, the fusion protein comprises the sequence set forth in SEQ ID NO:272, SEQ ID NO:273, SEQ ID NO:274, SEQ ID NO:275, SEQ ID NO:276, SEQ ID NO:277, or SEQ ID NO:278.

In some of any embodiments, the DNA-targeting domain is capable of hybridizing to the target site or is complementary to the target site at the endogenous locus.

In some of any embodiments, the at least one gRNA is capable of hybridizing to the target site or is complementary to the target site at the endogenous locus.

In some of any embodiments, the target site is located at a regulatory DNA element of the endogenous locus. In some of any embodiments, the regulatory DNA element is selected from among a promoter, an upstream regulatory element, an enhancer, an exon, an intron, a 5′ untranslated region (UTR), a 3′ UTR, or a downstream regulatory element.

In some of any of the embodiments, the endogenous locus is in a human cell. In some of any of the embodiments, the endogenous locus is in a stem cell; liver cell, optionally a hepatocyte; muscle cell; heart cell, optionally a cardiomyocyte; brain cell, optionally a neuron; blood cell; immune cell, optionally a lymphoid cell, optionally a T cell; or a cell derived from any of the foregoing.

In some of any embodiments, the endogenous locus is FXN. In some of any embodiments, the target site is located within the genomic coordinates hg38 chr9:68, 940, 179-69, 205, 519 or hg38 chr9:69, 027, 282-69, 028, 497. In some of any embodiments, the target site is located within the genomic coordinates hg38 chr9:69, 027, 615-69, 028, 101. In some of any embodiments, the target site comprises a sequence set forth in any one of SEQ ID NOS:208-228. In some of any embodiments, the target site comprises a sequence set forth in SEQ ID NO:208. In some of any embodiments, the target site comprises a sequence set forth in SEQ ID NO:214. In some of any embodiments, the target site comprises a sequence set forth in SEQ ID NO:228.

Also provided are DNA-targeting systems comprising any of the provided fusion proteins.

Also provided are DNA-targeting systems comprising any of the provided fusion proteins, and at least one gRNA.

Also provided are DNA-targeting systems comprising a DNA-targeting domain, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. Also provided are DNA-targeting systems comprising a DNA-targeting domain, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2. In some of any embodiments, the transcriptional activation domain increases transcription of an endogenous locus when recruited to a target site at the endogenous locus.

In some of any embodiments, the DNA-targeting domain comprises a Cas-gRNA combination comprising a Cas protein or a variant thereof, and at least one gRNA that binds to the target site at the endogenous locus.

In some of any embodiments, the DNA-targeting domain comprises a zinc finger protein (ZFP), a transcription activator-like effector (TALE), a meganuclease, a homing endonuclease, or an I-SceI enzyme or a variant thereof that binds to the target site at the endogenous locus.

Also provided are DNA-targeting systems comprising a Cas-gRNA combination comprising a Cas protein or a variant thereof, and at least one gRNA that binds to the target site at an endogenous locus, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. Also provided are DNA-targeting systems comprising a Cas-gRNA combination comprising a Cas protein or a variant thereof, and at least one gRNA that binds to the target site at an endogenous locus, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2. In some of any embodiments, the transcriptional activation domain increases transcription of an endogenous locus when recruited to a target site at the endogenous locus.

Also provided are DNA-targeting systems comprising a zinc finger protein (ZFP), a transcription activator-like effector (TALE), a meganuclease, a homing endonuclease, or an I-SceI enzyme or a variant thereof that binds to the target site at the endogenous locus, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. Also provided are DNA-targeting systems comprising a zinc finger protein (ZFP), a transcription activator-like effector (TALE), a meganuclease, a homing endonuclease, or an I-SceI enzyme or a variant thereof that binds to the target site at the endogenous locus, and two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2. In some of any embodiments, the transcriptional activation domain increases transcription of an endogenous locus when recruited to a target site at the endogenous locus.

In some of any embodiments, the Cas protein or a variant thereof, and the two or more transcriptional activation domains are fused in a fusion protein.

In some of any embodiments, the a zinc finger protein (ZFP), a transcription activator-like effector (TALE), a meganuclease, a homing endonuclease, or an I-SceI enzyme or a variant thereof, and the two or more transcriptional activation domains are fused in a fusion protein.

In some of any embodiments, the Cas protein or a variant thereof is a Cas9 or a variant thereof. In some of any embodiments, the Cas protein or a variant thereof protein is a deactivated Cas9 (dCas9).

In some of any embodiments, the Cas protein or a variant thereof is a Staphylococcus aureus Cas9 (SaCas9) or a variant thereof. In some of any embodiments, the Cas protein or a variant thereof is a Staphylococcus aureus dCas9 (dSaCas9) that comprises at least one amino acid mutation selected from D10A and N580A, with reference to numbering of positions of SEQ ID NO:3. In some of any embodiments, the Cas protein or a variant thereof comprises the sequence set forth in SEQ ID NO:2, and an amino acid sequence that has at least 90%, 91%, 92%, 93%, 94%, 95%, 96%, 97%, 98%, and 99% sequence identity thereto.

In some of any embodiments, the Cas9 or variant thereof is a Streptococcus pyogenes Cas9 (SpCas9) or a variant thereof. In some of any embodiments, the Cas protein or a variant thereof is a Streptococcus pyogenes dCas9 (dSpCas9) that comprises at least one amino acid mutation selected from D10A and H840A, with reference to numbering of positions of SEQ ID NO:7. In some of any embodiments, the Cas protein or a variant thereof comprises the sequence set forth in SEQ ID NO:6, and an amino acid sequence that has at least 90%, 91%, 92%, 93%, 94%, 95%, 96%, 97%, 98%, and 99% sequence identity thereto.

In some of any embodiments, the Cas protein or a variant thereof is a split variant Cas protein, wherein the split variant Cas protein comprises a first polypeptide comprising an N-terminal fragment of the variant Cas protein and an N-terminal Intein, and a second polypeptide comprising a C-terminal fragment of the variant Cas protein and a C-terminal Intein. In some of any embodiments, when the first polypeptide and the second polypeptide of the split variant Cas protein are present in proximity or present in the same cell, the N-terminal Intein and C-terminal Intein self-excise and ligate the N-terminal fragment and the C-terminal fragment of the variant Cas protein to form a full-length variant Cas protein. In some of any embodiments, the N-terminal Intein comprises an N-terminal Npu Intein, or the sequence set forth in SEQ ID NO:88, or an amino acid sequence that has at least 90%, 91%, 92%, 93%, 94%, 95%, 96%, 97%, 98%, or 99% sequence identity thereto, or a portion of any of the foregoing. In some of any embodiments, N-terminal fragment of the variant Cas protein comprises: the N-terminal fragment of variant SpCas9 from the N-terminal end up to position 573 of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or the sequence set forth in SEQ ID NO:86, or an amino acid sequence that has at least 90%, 91%, 92%, 93%, 94%, 95%, 96%, 97%, 98%, or 99% sequence identity thereto, or a portion of any of the foregoing. In some of any embodiments, the C-terminal Intein comprises a C-terminal Npu Intein, or the sequence set forth in SEQ ID NO:92, or an amino acid sequence that has at least 90%, 91%, 92%, 93%, 94%, 95%, 96%, 97%, 98%, or 99% sequence identity thereto, or a portion of any of the foregoing. In some of any embodiments, C-terminal fragment of the variant Cas protein comprises: the C-terminal fragment of variant SpCas9 from position 574 to the C-terminal end of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or the sequence set forth in SEQ ID NO:94, or an amino acid sequence that has at least 90%, 91%, 92%, 93%, 94%, 95%, 96%, 97%, 98%, or 99% sequence identity thereto, or a portion of any of the foregoing.

In some of any embodiments, the Cas protein or a variant thereof is a Cpf1 or a variant thereof. In some of any embodiments, the Cas protein or a variant thereof is a variant Cpf1 that that is a deactivated Cpf1 (dCpf1). In some of any embodiments, the variant comprises a catalytically inactive nuclease variant.

In some of any embodiments, the transcriptional activation domain of NCOA3 comprises: (i) the sequence set forth in SEQ ID NO:40; (ii) a contiguous portion of SEQ ID NO:40 of at least 20 amino acids; (iii) the sequence set forth in SEQ ID NO:27; (iv) a contiguous portion of SEQ ID NO:27 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of NCOA3 comprises the sequence set forth in SEQ ID NO:133 or an amino acid sequence that has at least 90%, 91%, 92%, 93%, 94%, 95%, 96%, 97%, 98%, or 99% sequence identity to SEQ ID NO:133. In some of any embodiments, the transcriptional activation domain of NCOA3 comprises the sequence set forth in SEQ ID NO:133.

In some of any embodiments, the transcriptional activation domain of ENL comprises: (i) the sequence set forth in SEQ ID NO:36; (ii) a contiguous portion of SEQ ID NO:36 of at least 20 amino acids; (iii) the sequence set forth in SEQ ID NO:23; (iv) a contiguous portion of SEQ ID NO:23 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of ENL comprises the sequence set forth in SEQ ID NO:131 or an amino acid sequence that has at least 90%, 91%, 92%, 93%, 94%, 95%, 96%, 97%, 98%, or 99% sequence identity to any of the foregoing. In some of any embodiments, the transcriptional activation domain of ENL comprises the sequence set forth in SEQ ID NO:131.

In some of any embodiments, the transcriptional activation domain of FOXO3 comprises: (i) the sequence set forth in SEQ ID NO:37; (ii) a contiguous portion of SEQ ID NO:37 of at least 20 amino acids; (iii) the sequence set forth in SEQ ID NO:24; (iv) a contiguous portion of SEQ ID NO:24 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of FOXO3 comprises the sequence set forth in SEQ ID NO:132 or an amino acid sequence that has at least 90%, 91%, 92%, 93%, 94%, 95%, 96%, 97%, 98%, or 99% sequence identity to any of the foregoing. In some of any embodiments, the transcriptional activation domain of FOXO3 comprises the sequence set forth in SEQ ID NO:132.

In some of any embodiments, the transcriptional activation domain of PYGO1 comprises: (i) the sequence set forth in SEQ ID NO:42; (ii) a contiguous portion of SEQ ID NO:42 of at least 20 amino acids; (iii) the sequence set forth in SEQ ID NO:29; (iv) a contiguous portion of SEQ ID NO:29 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of PYGO1 comprises the sequence set forth in SEQ ID NO:130 or an amino acid sequence that has at least 90%, 91%, 92%, 93%, 94%, 95%, 96%, 97%, 98%, or 99% sequence identity to any of the foregoing. In some of any embodiments, the transcriptional activation domain of PYGO1 comprises the sequence set forth in SEQ ID NO:130.

In some of any embodiments, the transcriptional activation domain of HSH2D comprises: (i) the sequence set forth in SEQ ID NO:38; (ii) a contiguous portion of SEQ ID NO:38 of at least 20 amino acids; (iii) the sequence set forth in SEQ ID NO:25; (iv) a contiguous portion of SEQ ID NO:25 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of HSH2D comprises the sequence set forth in SEQ ID NO:134 or an amino acid sequence that has at least 90%, 91%, 92%, 93%, 94%, 95%, 96%, 97%, 98%, or 99% sequence identity to any of the foregoing. In some of any embodiments, the transcriptional activation domain of HSH2D comprises the sequence set forth in SEQ ID NO:134.

In some of any embodiments, the transcriptional activation domain of NCOA2 comprises: (i) the sequence set forth in SEQ ID NO:39; (ii) a contiguous portion of SEQ ID NO:39 of at least 20 amino acids; (iii) the sequence set forth in SEQ ID NO:26; (iv) a contiguous portion of SEQ ID NO:26 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of NCOA2 comprises the sequence set forth in SEQ ID NO:135 or an amino acid sequence that has at least 90%, 91%, 92%, 93%, 94%, 95%, 96%, 97%, 98%, or 99% sequence identity to any of the foregoing. In some of any embodiments, the transcriptional activation domain of NCOA2 comprises the sequence set forth in SEQ ID NO:135.

In some of any embodiments, the transcriptional activation domain of NOTCH2 comprises: (i) the sequence set forth in SEQ ID NO:46; (ii) a contiguous portion of SEQ ID NO:46 of at least 20 amino acids; (iii) the sequence set forth in SEQ ID NO:33; (iv) a contiguous portion of SEQ ID NO:33 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of NOTCH2 comprises the sequence set forth in SEQ ID NO:136 or an amino acid sequence that has at least 90%, 91%, 92%, 93%, 94%, 95%, 96%, 97%, 98%, or 99% sequence identity to any of the foregoing. In some of any embodiments, the transcriptional activation domain of NOTCH2 comprises the sequence set forth in SEQ ID NO:136.

In some of any embodiments, the transcriptional activation domain of DPOLA comprises: (i) the sequence set forth in SEQ ID NO:35; (ii) a contiguous portion of SEQ ID NO:35 of at least 20 amino acids; (iii) the sequence set forth in SEQ ID NO:22; (iv) a contiguous portion of SEQ ID NO:22 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of DPOLA comprises the sequence set forth in SEQ ID NO:176 or an amino acid sequence that has at least 90%, 91%, 92%, 93%, 94%, 95%, 96%, 97%, 98%, or 99% sequence identity to any of the foregoing. In some of any embodiments, the transcriptional activation domain of DPOLA comprises the sequence set forth in SEQ ID NO:176.

In some of any embodiments, the transcriptional activation domain of PSA1 comprises: (i) the sequence set forth in SEQ ID NO:41; (ii) a contiguous portion of SEQ ID NO:41 of at least 20 amino acids; (iii) the sequence set forth in SEQ ID NO:28; (iv) a contiguous portion of SEQ ID NO:28 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of PSA1 comprises the sequence set forth in SEQ ID NO:177 or an amino acid sequence that has at least 90%, 91%, 92%, 93%, 94%, 95%, 96%, 97%, 98%, or 99% sequence identity to any of the foregoing. In some of any embodiments, the transcriptional activation domain of PSA1 comprises the sequence set forth in SEQ ID NO:177.

In some of any embodiments, the transcriptional activation domain of RBM39 comprises: (i) the sequence set forth in SEQ ID NO:43; (ii) a contiguous portion of SEQ ID NO:43 of at least 20 amino acids; (iii) the sequence set forth in SEQ ID NO:30; (iv) a contiguous portion of SEQ ID NO:30 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of RBM39 comprises the sequence set forth in SEQ ID NO:178 or an amino acid sequence that has at least 90%, 91%, 92%, 93%, 94%, 95%, 96%, 97%, 98%, or 99% sequence identity to any of the foregoing. In some of any embodiments, the transcriptional activation domain of RBM39 comprises the sequence set forth in SEQ ID NO:178.

In some of any embodiments, the transcriptional activation domain of HERC2 comprises: (i) the sequence set forth in SEQ ID NO:44; (ii) a contiguous portion of SEQ ID NO:44 of at least 20 amino acids; (iii) the sequence set forth in SEQ ID NO:31; (iv) a contiguous portion of SEQ ID NO:31 of at least 20 amino acids; (v) an amino acid sequence that has at least 90%, 91%, 92%, 93%, 94%, 95%, 96%, 97%, 98%, or 99% sequence identity to any of the foregoing. In some of any embodiments, the transcriptional activation domain of HERC2 comprises the sequence set forth in SEQ ID NO:179 or an amino acid sequence that has at least 90%, 91%, 92%, 93%, 94%, 95%, 96%, 97%, 98%, or 99% sequence identity to any of the foregoing. In some of any embodiments, the transcriptional activation domain of HERC2 comprises the sequence set forth in SEQ ID NO:179.

In some of any embodiments, the transcriptional activation domain is at least at or about 30, 40, 50, 60, or 70 amino acids in length. In some of any embodiments, the transcriptional activation domain is at least at or about 40 amino acids in length. In some of any embodiments, the transcriptional activation domain is at least at or about 50 amino acids in length. In some of any embodiments, the transcriptional activation domain is at least at or about 60 amino acids in length. In some of any embodiments, the transcriptional activation domain is at least at or about 70 amino acids in length.

In some of any embodiments, the transcriptional activation domain is at or about 120, 110, 100, 90, 80, 70, 60, 50, or 40 amino acids or less in length. In some of any embodiments, the transcriptional activation domain is 70 amino acids or less in length. In some of any embodiments, the transcriptional activation domain is 60 amino acids or less in length. In some of any embodiments, the transcriptional activation domain is 50 amino acids or less in length.

In some of any embodiments, the transcriptional activation domain is between at or about 40 and at or about 120, at or about 40 and at or about 110, at or about 40 and at or about 100, at or about 40 and at or about 90, at or about 40 and at or about 80, at or about 40 and at or about 70, at or about 40 and at or about 60, or at or about 40 and at or about 50 in length.

In some of any embodiments, the two or more transcriptional activation domains is two transcriptional activation domains.

In some of any embodiments, the fusion protein comprises: a transcriptional activation domain of PYGO1 and a transcriptional activation domain of NCOA3; a transcriptional activation domain of NOTCH2 and a transcriptional activation domain of NCOA3; a transcriptional activation domain of NCOA3 and a transcriptional activation domain of NCOA3; a transcriptional activation domain of HSH2D and a transcriptional activation domain of NCOA3; a transcriptional activation domain of FOXO3 and a transcriptional activation domain of NCOA3; a transcriptional activation domain of NCOA2 and a transcriptional activation domain of NCOA3; a transcriptional activation domain of ENL and a transcriptional activation domain of NCOA3; a transcriptional activation domain of PYGO1 and a transcriptional activation domain of FOXO3; a transcriptional activation domain of NOTCH2 and a transcriptional activation domain of FOXO3; a transcriptional activation domain of NCOA3 and a transcriptional activation domain of FOXO3; a transcriptional activation domain of HSH2D and a transcriptional activation domain of FOXO3; a transcriptional activation domain of FOXO3 and a transcriptional activation domain of FOXO3; a transcriptional activation domain of NCOA2 and a transcriptional activation domain of FOXO3; or a transcriptional activation domain of ENL and a transcriptional activation domain of FOXO3.

In some of any embodiments, the fusion protein comprises, in N-terminus to C-terminus order: a transcriptional activation domain of PYGO1, a linker, and a transcriptional activation domain of NCOA3; a transcriptional activation domain of NOTCH2, a linker, and a transcriptional activation domain of NCOA3; a transcriptional activation domain of NCOA3, a linker, and a transcriptional activation domain of NCOA3; a transcriptional activation domain of HSH2D, a linker, and a transcriptional activation domain of NCOA3; a transcriptional activation domain of FOXO3, a linker, and a transcriptional activation domain of NCOA3; a transcriptional activation domain of NCOA2, a linker, and a transcriptional activation domain of NCOA3; a transcriptional activation domain of ENL, a linker, and a transcriptional activation domain of NCOA3; a transcriptional activation domain of PYGO1, a linker, and a transcriptional activation domain of FOXO3; a transcriptional activation domain of NOTCH2, a linker, and a transcriptional activation domain of FOXO3; a transcriptional activation domain of NCOA3, a linker, and a transcriptional activation domain of FOXO3; a transcriptional activation domain of HSH2D, a linker, and a transcriptional activation domain of FOXO3; a transcriptional activation domain of FOXO3, a linker, and a transcriptional activation domain of FOXO3; a transcriptional activation domain of NCOA2, a linker, and a transcriptional activation domain of FOXO3; or a transcriptional activation domain of ENL, a linker, and a transcriptional activation domain of FOXO3.

In some of any embodiments, the fusion protein comprises the sequence set forth in any one of SEQ ID NOS:140-153, or an amino acid sequence that has at least 90%, 91%, 92%, 93%, 94%, 95%, 96%, 97%, 98%, or 99% sequence identity to any of the foregoing. In some of any embodiments, the fusion protein comprises the sequence set forth in SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, or SEQ ID NO:153.

In some of any embodiments, the fusion protein comprises, in N-terminus to C-terminus order: a dCas9, a transcriptional activation domain of PYGO1 and a transcriptional activation domain of NCOA3; a dCas9, a transcriptional activation domain of NOTCH2 and a transcriptional activation domain of NCOA3; a dCas9, a transcriptional activation domain of NCOA3 and a transcriptional activation domain of NCOA3; a dCas9, a transcriptional activation domain of HSH2D and a transcriptional activation domain of NCOA3; a dCas9, a transcriptional activation domain of FOXO3 and a transcriptional activation domain of NCOA3; a dCas9, a transcriptional activation domain of NCOA2 and a transcriptional activation domain of NCOA3; a dCas9, a transcriptional activation domain of ENL and a transcriptional activation domain of NCOA3; a dCas9, a transcriptional activation domain of PYGO1 and a transcriptional activation domain of FOXO3; a dCas9, a transcriptional activation domain of NOTCH2 and a transcriptional activation domain of FOXO3; a dCas9, a transcriptional activation domain of NCOA3 and a transcriptional activation domain of FOXO3; a dCas9, a transcriptional activation domain of HSH2D and a transcriptional activation domain of FOXO3; a dCas9, a transcriptional activation domain of FOXO3 and a transcriptional activation domain of FOXO3; a dCas9, a transcriptional activation domain of NCOA2 and a transcriptional activation domain of FOXO3; or a dCas9, a transcriptional activation domain of ENL and a transcriptional activation domain of FOXO3.

In some of any embodiments, the fusion protein comprises, in N-terminus to C-terminus order: a transcriptional activation domain of PYGO1, a transcriptional activation domain of NCOA3, and a dCas9; a transcriptional activation domain of NOTCH2, a transcriptional activation domain of NCOA3, and a dCas9; a transcriptional activation domain of NCOA3, a transcriptional activation domain of NCOA3, and a dCas9; a transcriptional activation domain of HSH2D, a transcriptional activation domain of NCOA3, and a dCas9; a transcriptional activation domain of FOXO3, a transcriptional activation domain of NCOA3, and a dCas9; a transcriptional activation domain of NCOA2, a transcriptional activation domain of NCOA3, and a dCas9; a transcriptional activation domain of ENL, a transcriptional activation domain of NCOA3, and a dCas9; a transcriptional activation domain of PYGO1, a transcriptional activation domain of FOXO3, and a dCas9; a transcriptional activation domain of NOTCH2, a transcriptional activation domain of FOXO3, and a dCas9; a transcriptional activation domain of NCOA3, a transcriptional activation domain of FOXO3, and a dCas9; a transcriptional activation domain of HSH2D, a transcriptional activation domain of FOXO3, and a dCas9; a transcriptional activation domain of FOXO3, a transcriptional activation domain of FOXO3, and a dCas9; a transcriptional activation domain of NCOA2, a transcriptional activation domain of FOXO3, and a dCas9; or a transcriptional activation domain of ENL, a transcriptional activation domain of FOXO3, and a dCas9.

In some of any embodiments, the two or more transcriptional activation domains is three transcriptional activation domains.

In some of any embodiments, the fusion protein comprises: a transcriptional activation domain of PYGO1, a transcriptional activation domain of FOXO3, and a transcriptional activation domain of NCOA3; a transcriptional activation domain of NOTCH2, a transcriptional activation domain of FOXO3, and a transcriptional activation domain of NCOA3; a transcriptional activation domain of NCOA3, a transcriptional activation domain of FOXO3, and a transcriptional activation domain of NCOA3; a transcriptional activation domain of HSH2D, a transcriptional activation domain of FOXO3, and a transcriptional activation domain of NCOA3; a transcriptional activation domain of FOXO3, a transcriptional activation domain of FOXO3, and a transcriptional activation domain of NCOA3; a transcriptional activation domain of NCOA2, a transcriptional activation domain of FOXO3, and a transcriptional activation domain of NCOA3; or a transcriptional activation domain of ENL, a transcriptional activation domain of FOXO3, and a transcriptional activation domain of NCOA3.

In some of any embodiments, the fusion protein comprises, in N-terminus to C-terminus order: a transcriptional activation domain of PYGO1, a linker, a transcriptional activation domain of FOXO3, a linker, and a transcriptional activation domain of NCOA3; a transcriptional activation domain of NOTCH2, a linker, a transcriptional activation domain of FOXO3, a linker, and a transcriptional activation domain of NCOA3; a transcriptional activation domain of NCOA3, a linker, a transcriptional activation domain of FOXO3, a linker, and a transcriptional activation domain of NCOA3; a transcriptional activation domain of HSH2D, a linker, a transcriptional activation domain of FOXO3, a linker, and a transcriptional activation domain of NCOA3; a transcriptional activation domain of FOXO3, a linker, a transcriptional activation domain of FOXO3, a linker, and a transcriptional activation domain of NCOA3; a transcriptional activation domain of NCOA2, a linker, a transcriptional activation domain of FOXO3, a linker, and a transcriptional activation domain of NCOA3; or a transcriptional activation domain of ENL, a linker, a transcriptional activation domain of FOXO3, a linker, and a transcriptional activation domain of NCOA3.

In some of any embodiments, the fusion protein comprises the sequence set forth in any one of SEQ ID NOS:154-160 or 377 or an amino acid sequence that has at least 90%, 91%, 92%, 93%, 94%, 95%, 96%, 97%, 98%, or 99% sequence identity to any of the foregoing.

In some of any embodiments, the fusion protein comprises the sequence set forth in SEQ ID NO:154, SEQ ID NO:155, SEQ ID NO:156, SEQ ID NO:157, SEQ ID NO:158, SEQ ID NO:159, or SEQ ID NO:160 or SEQ ID NO: 377.

In some of any embodiments, the fusion protein comprises a transcriptional activation domain of PYGO1, a linker, a transcriptional activation domain of FOXO3, a linker, and a transcriptional activation domain of NCOA3. In some of any embodiments, the fusion protein comprises the sequence set forth in SEQ ID NO:154.

In some of any embodiments, the fusion protein comprises a transcriptional activation domain of NCOA3, a linker, a transcriptional activation domain of FOXO3, a linker, and a transcriptional activation domain of NCOA3. In some of any embodiments, the fusion protein comprises the sequence set forth in SEQ ID NO:156.

In some of any embodiments, the fusion protein comprises a transcriptional activation domain of FOXO3, a linker, a transcriptional activation domain of FOXO3, a linker, and a transcriptional activation domain of NCOA3. In some of any embodiments, the fusion protein comprises the sequence set forth in SEQ ID NO:158.

In some of any embodiments, the fusion protein comprises a transcriptional activation domain of NCOA2, a linker, a transcriptional activation domain of FOXO3, a linker, and a transcriptional activation domain of NCOA3. In some of any embodiments, the fusion protein comprises the sequence set forth in SEQ ID NO:159.

In some embodiments, the multipartite activator comprises domains from NCOA3, FOXO3, and FOX03, respectively. In some embodiments, the multipartite activator comprises SEQ ID NO:377, or an amino acid sequence that has at least 90%, 91%, 92%, 93%, 94%, 95%, 96%, 97%, 98%, or 99% sequence identity to SEQ ID NO:377. In some embodiments, the multipartite activator is set forth in SEQ ID NO:377.

In some of any embodiments, the fusion protein comprises, in N-terminus to C-terminus order: a dCas9, a linker, a transcriptional activation domain of PYGO1, a linker, a transcriptional activation domain of FOXO3, a linker, and a transcriptional activation domain of NCOA3; a dCas9, a linker, a transcriptional activation domain of NOTCH2, a linker, a transcriptional activation domain of FOXO3, a linker, and a transcriptional activation domain of NCOA3; a dCas9, a linker, a transcriptional activation domain of NCOA3, a linker, a transcriptional activation domain of FOXO3, a linker, and a transcriptional activation domain of NCOA3; a dCas9, a linker, a transcriptional activation domain of HSH2D, a linker, a transcriptional activation domain of FOXO3, a linker, and a transcriptional activation domain of NCOA3; a dCas9, a linker, a transcriptional activation domain of FOXO3, a linker, a transcriptional activation domain of FOXO3, a linker, and a transcriptional activation domain of NCOA3; a dCas9, a linker, a transcriptional activation domain of NCOA2, a linker, a transcriptional activation domain of FOXO3, a linker, and a transcriptional activation domain of NCOA3; or a dCas9, a linker, a transcriptional activation domain of ENL, a linker, a transcriptional activation domain of FOXO3, a linker, and a transcriptional activation domain of NCOA3.

In some of any embodiments, the fusion protein comprises, in N-terminus to C-terminus order: a transcriptional activation domain of PYGO1, a linker, a transcriptional activation domain of FOXO3, a linker, a transcriptional activation domain of NCOA3, a linker, and a dCas9; a transcriptional activation domain of NOTCH2, a linker, a transcriptional activation domain of FOXO3, a linker, a transcriptional activation domain of NCOA3, a linker, and a dCas9; a transcriptional activation domain of NCOA3, a linker, a transcriptional activation domain of FOXO3, a linker, a transcriptional activation domain of NCOA3, a linker, and a dCas9; a transcriptional activation domain of HSH2D, a linker, a transcriptional activation domain of FOXO3, a linker, a transcriptional activation domain of NCOA3, a linker, and a dCas9; a transcriptional activation domain of FOXO3, a linker, a transcriptional activation domain of FOXO3, a linker, a transcriptional activation domain of NCOA3, a linker, and a dCas9; a transcriptional activation domain of NCOA2, a linker, a transcriptional activation domain of FOXO3, a linker, a transcriptional activation domain of NCOA3, a linker, and a dCas9; or a transcriptional activation domain of ENL, a linker, a transcriptional activation domain of FOXO3, a linker, a transcriptional activation domain of NCOA3, a linker, and a dCas9.

In some of any embodiments, the two or more transcriptional activation domains comprises four transcriptional activation domains. In some of any embodiments, the two or more transcriptional activation domains comprises five transcriptional activation domains.

In some of any embodiments, the fusion proteins also include one or more linkers. In some of any embodiments, a linker of one or more linkers is positioned between the two or more transcriptional activation domains and/or positioned between the polypeptide component of the DNA-targeting domain and one of the two or more transcriptional activation domains. In some of any embodiments, the linker is a polypeptide linker. In some of any embodiments, the polypeptide linker comprises a sequence selected from among SEQ ID NOS:62-67, 96, and 137-139.

In some of any embodiments, the fusion proteins also include one or more nuclear localization signals (NLSs). In some of any embodiments, the one or more NLSs comprises two or more NLSs. In some of any embodiments, a NLS of one or more NLSs is positioned between the two or more transcriptional activation domains. In some of any embodiments, a NLS of the one or more NLSs is positioned between the polypeptide component of the DNA-targeting domain and one of the two or more transcriptional activation domains. In some of any embodiments, the one or more NLSs comprises a sequence selected from among SEQ ID NOS:69-84.

In some of any embodiments, the fusion protein comprises the sequence set forth in any one of SEQ ID NOS:181-187, or an amino acid sequence that has at least 90%, 91%, 92%, 93%, 94%, 95%, 96%, 97%, 98%, or 99% sequence identity to any of the foregoing. In some of any embodiments, the fusion protein comprises the sequence set forth in SEQ ID NO:181, SEQ ID NO:182, SEQ ID NO:183, SEQ ID NO:184, SEQ ID NO:185, SEQ ID NO:186, or SEQ ID NO:187.

In some of any embodiments, the fusion proteins also include a tag. In some of any embodiments, the tag comprises an epitope tag or a split protein tag. In some of any embodiments, the tag is selected from among SEQ ID NOS:61, 88, 92, and 167.

In some of any embodiments, the fusion protein comprises the sequence set forth in any one of SEQ ID NOS:272-278, or an amino acid sequence that has at least 90%, 91%, 92%, 93%, 94%, 95%, 96%, 97%, 98%, or 99% sequence identity to any of the foregoing. In some of any embodiments, the fusion protein comprises the sequence set forth in SEQ ID NO:272, SEQ ID NO:273, SEQ ID NO:274, SEQ ID NO:275, SEQ ID NO:276, SEQ ID NO:277, or SEQ ID NO:278.

In some of any embodiments, the DNA-targeting domain is capable of hybridizing to the target site or is complementary to the target site at the endogenous locus.

In some of any embodiments, the at least one gRNA is capable of hybridizing to the target site or is complementary to the target site at the endogenous locus.

In some of any embodiments, the target site is located at a regulatory DNA element of the endogenous locus. In some of any embodiments, the regulatory DNA element is selected from among a promoter, an upstream regulatory element, an enhancer, an exon, an intron, a 5′ untranslated region (UTR), a 3′ UTR, or a downstream regulatory element.

In some of any embodiments, the endogenous locus is FXN. In some of any embodiments, the target site is located within the genomic coordinates hg38 chr9:68, 940, 179-69, 205, 519 or hg38 chr9:69, 027, 282-69, 028, 497. In some of any embodiments, the target site is located within the genomic coordinates hg38 chr9:69, 027, 615-69, 028, 101. In some of any embodiments, the target site comprises a sequence set forth in any one of SEQ ID NOS:208-228. In some of any embodiments, the target site comprises a sequence set forth in SEQ ID NO:208. In some of any embodiments, the target site comprises a sequence set forth in SEQ ID NO:214. In some of any embodiments, the target site comprises a sequence set forth in SEQ ID NO:228.

In some of any embodiments, the gRNA comprises a sequence set forth in any one of SEQ ID NOS:229-249. In some of any embodiments, the gRNA comprises a sequence set forth in SEQ ID NO:229. In some of any embodiments, the gRNA comprises a sequence set forth in SEQ ID NO:235. In some of any embodiments, the gRNA comprises a sequence set forth in SEQ ID NO:249.

Also provided are polynucleotides comprising a sequence encoding any of the provided fusion proteins or any of the provided DNA-targeting systems, or a portion or a component of any of the foregoing.

In some of any embodiments, the sequence encoding the fusion protein comprises a sequence set forth in any one of SEQ ID NOS:109-122, or a nucleic acid sequence that has at least 90%, 91%, 92%, 93%, 94%, 95%, 96%, 97%, 98%, or 99% sequence identity to any of the foregoing. In some of any embodiments, the sequence encoding the fusion protein comprises a sequence set forth in any one of SEQ ID NOS:109-122.

In some of any embodiments, the sequence encoding the fusion protein comprises a sequence set forth in any one of SEQ ID NOS:123-129, or a nucleic acid sequence that has at least 90%, 91%, 92%, 93%, 94%, 95%, 96%, 97%, 98%, or 99% sequence identity to any of the foregoing. In some of any embodiments, the sequence encoding the fusion protein comprises a sequence set forth in any one of SEQ ID NOS:123-129.

Also provided are pluralities of polynucleotides, comprising a first polynucleotide comprising any of the provided polynucleotides, and one or more second polynucleotides encoding an additional portion or an additional component of any of the provided fusion proteins or any of the provided DNA-targeting systems, or a portion or a component of any of the foregoing.

Also provided are vectors comprising any of the provided polynucleotides.

Also provided are vectors comprising any of the provided pluralities of polynucleotides.

In some of any embodiments, the vector is a viral vector. In some of any embodiments, the viral vector is an AAV vector. In some of any embodiments, the AAV vector is selected from among AAV1, AAV2, AAV3, AAV4, AAV5, AAV6, AAV7, AAV8, AAV9, AAV10, AAV11, AAV12, or AAV-DJ vector. In some of any embodiments, the AAV vector is an AAV5 vector or an AAV9 vector. In some of any embodiments, the viral vector is an AAV9 vector.

In some of any embodiments, the vector is a non-viral vector selected from: a lipid nanoparticle, a liposome, an exosome, or a cell penetrating peptide.

Also provided are pluralities of vectors, comprising a first vector comprising any of the provided vectors, and one or more second vectors comprising the one or more second polynucleotide of any of the provided pluralities of polynucleotides.

Also provided are cells comprising any of the provided fusion proteins, any of the provided DNA-targeting systems, any of the provided polynucleotides, any of the provided pluralities of polynucleotides, any of the provided vectors, or any of the provided pluralities of vectors, or a portion or a component of any of the foregoing.

Also provided are methods for modulating the expression of an endogenous locus in a cell. In some of any embodiments, the methods involve introducing any of the provided fusion proteins, any of the provided DNA-targeting systems, any of the provided polynucleotides, any of the provided pluralities of polynucleotides, any of the provided vectors, or any of the provided pluralities of vectors, or a portion or a component of any of the foregoing, into the cell.

Also provided are methods for modulating the expression of an endogenous locus in a subject. In some of any embodiments, the methods involve administering any of the provided fusion proteins, any of the provided DNA-targeting systems-, any of the provided polynucleotides, any of the provided pluralities of polynucleotides, any of the provided vectors, or any of the provided pluralities of vectors, or a portion or a component of any of the foregoing, to the subject.

In some of any embodiments, the fusion protein or the DNA-targeting system increases transcription of an endogenous locus when recruited to a target site at the endogenous locus.

In some of any embodiments, the endogenous locus is FXN.

In some of any embodiments, the target site is located within the genomic coordinates hg38 chr9:68, 940, 179-69, 205, 519 or hg38 chr9:69, 027, 282-69, 028, 497. In some of any embodiments, the target site is located within the genomic coordinates hg38 chr9:69, 027, 615-69, 028, 101. In some of any embodiments, the target site comprises a sequence set forth in any one of SEQ ID NOS:208-228. In some of any embodiments, the target site comprises a sequence set forth in SEQ ID NO:208. In some of any embodiments, the target site comprises a sequence set forth in SEQ ID NO:214. In some of any embodiments, the target site comprises a sequence set forth in SEQ ID NO:228.

In some of any embodiments, the cell is from a subject that has or is suspected of having a disease or disorder or the subject has or is suspected of having a disease or disorder. In some of any embodiments, the disease or disorder is associated with the reduction of expression of the endogenous locus. In some of any embodiments, the introducing, contacting or administering is carried out in vivo or ex vivo. In some of any embodiments, the subject is a human.

Also provided are pharmaceutical compositions comprising any of the provided fusion proteins, any of the provided DNA-targeting systems, any of the provided polynucleotides, any of the provided pluralities of polynucleotides, any of the provided vectors, or any of the provided pluralities of vectors, or a portion or a component of any of the foregoing.

In some of any embodiments, the provided pharmaceutical compositions are for use in treating a disease or disorder. In some of any embodiments, the provided pharmaceutical compositions are for use in the manufacture of a medicament for treating a disease or disorder.

Also provided are uses of any of the provided pharmaceutical compositions for treating a disease or disorder.

Also provided are uses of any of the provided pharmaceutical compositions in the manufacture of a medicament for treating a disease or disorder. In some of any embodiments, the disease or disorder is associated with the reduction of expression of an endogenous locus.

In some of any embodiments, the pharmaceutical composition is to be administered to a subject. In some of any embodiments, the administration is carried out in vivo or ex vivo.

In some of any embodiments, the fusion protein or the DNA-targeting system increases transcription of an endogenous locus when recruited to a target site at the endogenous locus.

In some of any embodiments, the endogenous locus is FXN. In some of any embodiments, the target site is located within the genomic coordinates hg38 chr9:68, 940, 179-69, 205, 519 or hg38 chr9:69, 027, 282-69, 028, 497. In some of any embodiments, the target site is located within the genomic coordinates hg38 chr9:69, 027, 615-69, 028, 101. In some of any embodiments, the target site comprises a sequence set forth in any one of SEQ ID NOS:208-228. In some of any embodiments, the target site comprises a sequence set forth in SEQ ID NO:208. In some of any embodiments, the target site comprises a sequence set forth in SEQ ID NO:214. In some of any embodiments, the target site comprises a sequence set forth in SEQ ID NO:228.

In some of any embodiments, the subject is a human.

### BRIEF DESCRIPTION OF THE DRAWINGS

FIGS. 1A and 1B show scatterplots of results from sequencing analysis of screen for transcriptional activation domains. WT-iPSCs expressing a frataxin promoter-targeting gRNA were transduced with pooled libraries of fusion proteins comprising fragments of nuclear localized proteins, fused to the N-terminus (FIG. 1A) or C-terminus (FIG. 1B) of dSaCas9. Transduced cells were subsequently sorted by flow cytometry into populations representing top 10% and bottom 10% of cells based on frataxin protein expression. Populations were sequenced to identify protein fragments enriched in the frataxin-high or frataxin-low populations based on DESeq2. Each dot in the scatterplots represents a single protein fragment. y-axis represents log fold change in frataxin-high versus frataxin-low populations. x-axis represents mean of normalized counts. Enriched protein fragments are highlighted in red, as transcriptional activators (positive log fold change; enriched transcriptional activators are also circled) and transcriptional repressors (negative log fold change). The N-terminal screen identified 9 transcriptional activators and the C-terminal screen identified 5 transcriptional activators.

FIGS. 2A and 2B show FXN mRNA expression in wild-type iPSCs (WT-iPSCs) stably expressing the frataxin promoter-targeting gRNA and transduced with dSaCas9 fusion proteins comprising protein fragments from the indicated genes, as well as with positive control (2×VP64) and negative control (control peptide) dSaCas9 fusion proteins. FIG. 2A shows N-terminal fusions, FIG. 2B shows C-terminal fusions. Expression was assessed by RT-qPCR in comparison to negative control. Dots represent experimental replicates, bars represent mean.

FIGS. 3A and 3B show FXN mRNA expression in iPSCs generated from a subject with a GAA trinucleotide repeat expansion in the frataxin gene (FA-iPSCs) stably expressing the frataxin promoter-targeting gRNA and transduced with dSaCas9 fusion proteins comprising protein fragments from the indicated genes, as well as with positive control (2×VP64) and negative control (control peptide) dSaCas9 fusion proteins. FIG. 3A shows N-terminal fusions, FIG. 3B shows C-terminal fusions. Expression was assessed by RT-qPCR in comparison to negative control. Dots represent experimental replicates, bars represent mean.

FIGS. 4A and 4B show FXN mRNA expression in iPSCs expressing the frataxin promoter-targeting gRNA and transduced with dSaCas9 fusion proteins with the indicated multipartite activators, a positive control dSaCas9 fusion protein (2×VP64), a negative control dSaCas9 fusion protein (CTRLFRAG), or no fusion protein (puro control). FA-iPSCs were used for all conditions except for the condition labeled “WT-CTRLFRAG” in which WT-IPSCs were used. Expression was assessed by RT-qPCR in comparison to negative control. Dots represent experimental replicates, bars represent mean.

FIGS. 5A-5C show Nrf2 mRNA expression in N2a cells transfected with indicated fusion proteins and/or gRNAs. FIG. 5A shows Nrf2 mRNA expression in N2a cells transfected with dSaCas9-2×VP64 and no gRNA (negative control), or dSaCas9-2×VP64 with indicated individual or combined Nrf2-targeting gRNAs A, B, and C. FIG. 5B and FIG. 5C show Nrf2 mRNA expression in N2a cells transfected with dSaCas9-2×VP64 and no gRNA (negative control), or with Nrf2 gRNA B and dSaCas9 fusion proteins with a negative control fragment, 2×VP64 (positive control) or indicated multipartite activators. Expression was assessed by RT-qPCR in comparison to negative control (no gRNA). Dots represent experimental replicates, bars represent mean.

FIG. 6 shows a schematic illustrating an exemplary dSaCas9-tripartite effector fusion protein, with domains from FOXO3 and NCOA3. The first domain (labeled “effector”) can comprise different domains, as described in the Examples.

FIG. 7 shows frataxin protein expression in FA-iPSC-derived cardiomyocytes following AAV-DJ delivery of dSaCas9 fusion proteins with indicated FXN-targeting gRNA or non-targeting gRNA (NT). Boxes indicating “tripartite effectors” indicate conditions with dSaCas9 fusion proteins with tripartite effectors comprising the indicated domain (e.g. FOXO3, NCOA2, NCOA3, or PYGO1), followed by a domain from FOXO3 and NCOA3, in the N- to C-terminal direction, e.g. as illustrated in FIG. 6.

FIGS. 8A-8C shows results from FA-iPSC-derived cardiomyocytes following delivery of the indicated dSaCas9 fusion proteins and FXN-targeting gRNA. Shown are MOI versus % of WT FXN protein expression (FIG. 8A), VCN versus % of WT FXN protein expression (FIG. 8B), or a summary table of the results (FIG. 8C).

FIG. 9 shows VCN versus % of WT FXN protein expression levels in FA-iPSC-derived cardiomyocytes following delivery of dSaCas9 fusion proteins with the indicated effectors for transcriptional activation. Individual domain names (e.g. NCOA3) stand for tripartite effectors comprising the domain, followed by FOXO3 and NCOA3, e.g. as illustrated in FIG. 6.

FIGS. 10A and 10B show FXN protein expression levels (in comparison to WT control) in FA-iPSC-derived cardiomyocytes (FIG. 10A) or FA-iPSC-derived neurons (FIG. 10B) following delivery of dSaCas9 fusion proteins with the indicated effectors for transcriptional activation. Individual domain names (e.g. FOXO3, NCOA2, NCOA3) stand for tripartite effectors comprising the domain, followed by FOXO3 and NCOA3, e.g. as illustrated in FIG. 6.

FIG. 11 shows FXN mRNA expression levels (as compared to WT cells) in FA-iPSC-derived cardiomyocytes following AAVDJ delivery of a) the fusion proteins comprising an FXN-targeting eZFP and VP64 or the indicated tripartite effectors, or b) dSaCas9 fusion proteins comprising 2×VP64 or the indicated tripartite effectors with FXN-targeting gRNA.

FIG. 12 shows FXN mRNA expression levels in FA-iPSC-derived neurons following AAVDJ delivery of a) the fusion proteins comprising a FXN-targeting eZFP and VP64 or the indicated tripartite effectors, or b) dSaCas9 fusion proteins comprising 2×VP64 or the indicated tripartite effectors with FXN-targeting gRNA.

FIG. 13 shows FXN mRNA expression levels in FA-iPSC-derived neurons (as compared to WT control cells) following AAVDJ delivery of fusion proteins comprising FXN-targeting eZFP and indicated tripartite effectors fused to the C-terminus or N-terminus of the eZFP.

FIGS. 14A-C show IL-2 expression levels in CAR T cells following delivery of DNA-targeting systems comprising an IL-2-targeting gRNA and dSpCas9 fused to either a FOXO3-FOXO3-NCOA3 tripartite effector (dSpCas9-FFN) or an NCOA3-FOXO3-NCOA3 tripartite effector (dSpCas9-NFN). FIG. 14A shows schematics illustrating the delivered fusion proteins. FIG. 14B shows IL-2 secretion after a first, second, and third stimulation with target antigen-expressing target cells, as measured by immunoassay. FIG. 14C shows IL-2 mRNA expression at timepoints after delivery of the DNA-targeting systems, as measured by qRT-PCT.

FIGS. 15A-G show IL-2 expression levels in CAR T cells at various timepoints post-electroporation (post-EP) with DNA-targeting systems comprising dSpCas9-NFN and an IL-2-targeting gRNA. FIGS. 15A-F show IL-2 expression as measured by intracellular cytokine staining (ICS) and flow cytometry, quantified as mean fluorescence intensity (FIG. 15A-C for day 2, day 7, and day 14 post-EP, respectively), or quantified as percentage of cells identified as positive for IL-2 (FIG. 15D-F for day 2, day 7, and day 14 post-EP, respectively). FIG. 15G shows IL-2 expression at day 7 post-EP as measured by qRT-PCR.

FIG. 16 shows CCR7 expression in Jurkat cells delivered with dSpCas9-2×VP64 and a non-targeting gRNA or a CCR7-targeting gRNA, as measured by flow cytometry at day 2, day 6, and day 10 post-delivery. Populations delivered with the CCR7-targeting gRNA are comparatively higher CCR7 expression, which can be measured, for example, as percentage of positive CCR7+ cells, or as mean fluorescence intensity of the signal corresponding to CCR7 expression, as shown in the figure.

FIG. 17 shows schematics illustrating various fusion proteins used to generate results in FIGS. 18-20. The top two schematics show fusion proteins comprising dSpCas9 fused at its N terminal to a FOXO3-FOXO3-NCOA3 tripartite effector (FFN) or an NCOA3-FOXO3-NCOA3 tripartite effector (NFN). The bottom schematic shows a fusion protein comprising dSpCas9 fused to a repeating GCN4 epitope array (5×GCN4). The GCN4 epitopes are recognized by, and recruit an anti-GCN4 single chain variable fragment (scFv) domain, which can be fused, for example, to individual transcriptional activation domains or multipartite effectors.

FIGS. 18A-18B show CCR7 expression in Jurkat cells as measured by flow cytometry, quantified as mean fluorescence intensity (FIG. 18A) or percentage of CCR7 high cells (FIG. 18B), following delivery of a CCR7-targeting gRNA and indicated fusion protein(s).

FIG. 19 shows CCR7 expression in Jurkat cells as measured by flow cytometry and quantified as mean fluorescence intensity following delivery of DNA-targeting systems comprising a CCR7-targeting gRNA, a dSpCas9-5×GCN4 fusion protein, and the indicated effectors fused to a GCN4-targeting scFv domain. For each effector, two bars are shown, corresponding to 1 μg (left bar) or 2 μg (right bar) of the mRNA encoding the effector-scFv fusion protein being delivered.

FIGS. 20A-B show heatmaps representing CCR7 expression in Jurkat cells as measured by flow cytometry and quantified as mean fluorescence intensity following delivery of DNA-targeting systems comprising a CCR7-targeting gRNA, a dSpCas9-5×GCN4 fusion protein, and the indicated combinations of effector-scFv fusion proteins. Results are shown on linear scale (FIG. 20A) and log scale (FIG. 20B).

### DETAILED DESCRIPTION

Provided herein are fusion proteins comprising transcriptional activation domains, such as two or more transcriptional activation domains, for targeted transcriptional activation, for example at a target locus. Also provided are fusion proteins, effector proteins, such as multipartite effector proteins (including multipartite activators), and DNA-targeting systems, such as CRISPR/Cas-based DNA-targeting systems, that comprise two or more of the transcriptional activation domains. In some aspects, the provided DNA-targeting systems comprise an effector protein or fusion protein provided herein. In some aspects, the DNA-targeting systems comprise one or more gRNAs. In some aspects, the transcriptional activation domains, fusion proteins, effector proteins, and DNA-targeting systems leads to increased transcription of an endogenous gene, when recruited to a target site at the endogenous gene. In some aspects, provided are multipartite effectors for transcriptional activation, such as multipartite activators, and fusion proteins, comprising the two or more of the transcriptional activation domains. In some aspects, provided herein are DNA-targeting systems comprising the transcriptional activation domains, such as CRISPR-Cas-based DNA-targeting systems, that are capable of inducing targeted transcriptional activation of target genes, for example, when recruited to a target site at the target gene. In some aspects, provided herein are DNA-targeting systems comprising any of the provided fusion proteins, that are capable of inducing targeted transcriptional activation of target genes, for example, when recruited to a target site at the target gene. Also provided are polynucleotides, vectors, pluralities and combinations thereof, that encode the fusion proteins, effector proteins, DNA-targeting systems, gRNAs or components thereof. Also provided are cells, such as engineered cells, that encode the fusion proteins, effector proteins, DNA-targeting systems, gRNAs or components thereof. Also provided are methods and uses related to the provided compositions, for example in activating transcription of a target gene or modifying a phenotype of a cell, including in connection with therapeutic applications.

Provided herein are fusion proteins comprising two or more transcriptional activation domains. In some embodiments, the fusion protein comprises two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. In some embodiments, the two or more transcriptional activation domains are comprised in a multipartite activator, such as a bipartite activator (comprising 2 transcriptional activation domains), a tripartite activator (comprising 3 transcriptional activation domains), or a multipartite activator comprising 4, 5, 6, 7, 8, 9, 10, or more transcriptional activation domains. In some embodiments, the two or more transcriptional activation domains and/or multipartite activators herein are used for targeted transcriptional activation. In some embodiments, provided herein are fusion proteins and/or DNA-targeting systems comprising the transcriptional activation domains and/or the multipartite activators, and one or more DNA-targeting domains. In some embodiments, the DNA-targeting domain recruits the two or more transcriptional activation domains, and/or a multipartite activator to a target site of an endogenous locus, such as a target site for a gene, thereby increasing transcription of the endogenous locus.

Targeted epigenetic modulation is an approach for investigating biology and therapeutic applications. Sequence-specific DNA-targeting systems, such as zinc finger proteins, transcription-activator-like effectors, and CRISPR/Cas systems can be programmed by a user to target sequences of interest. These DNA-targeting systems can be used to recruit effector proteins such as transcriptional and epigenetic modulators to endogenous genomic loci, for example to activate or repress transcription of a target gene.

Despite the potential for targeted transcriptional activation as a therapeutic or investigative tool, the ability to activate transcription can be unpredictable or unreliable, and is dependent on the specific effectors that are recruited to particular target sites. Only a handful of transcriptional activation domains have been frequently used for targeted transcriptional activation (Adli, M. Nat. Commun. 9, 1911 (2018)). For a given target site for a gene of interest, some transcriptional activation domains may lead to increased transcription of the gene, and others may not. In addition, different transcriptional activation domains may lead to different levels of increased transcription, or may induce increased transcription for different amounts of time. For example, a transcriptional activation domain may only transiently increase transcription, or may induce durably (e.g. heritably) increased transcription. The effect of a given transcriptional activation domain being recruited to a particular target site on the transcription of a gene is also generally unpredictable. Often, a transcriptional activation domain must be tested at several target sites to identify a suitable target site for transcriptional activation of the gene of interest.

The predictability and degree of transcriptional activation can affect the therapeutic potential of the transcriptional activation. For example, weak transcriptional activation may not increase transcription of the target gene sufficiently to result in a therapeutic effect. In some cases, strong or durable transcriptional activation may lead to a therapeutic effect. Therapeutic potential for human subjects of some transcriptional activation domains may also be limited by the immunogenicity of the domain, for example if the domain is from a non-human organism (e.g. VP64). These challenges raise the need for an expanded and improved transcriptional activation domains and fusion proteins and DNA-targeting systems containing transcriptional activation domains for use targeted transcriptional activation.

Provided herein are transcriptional activation domains, multipartite effectors for transcriptional activation (e.g., multipartite activators) and fusion proteins comprising the transcriptional activation domains for targeted transcriptional activation. Also provided herein are fusion proteins and DNA-targeting systems that target the transcriptional activation domains and multipartite activators to specific target sites. In some aspects, the provided are an expanded set of domains for targeted transcriptional activation. In some aspects, the transcriptional activation domains are derived from human genes, thereby reducing potential for immunogenicity in human subjects. In some aspects, compared to domains derived from non-human organisms, such as virally-derived VP64, the provided transcriptional activation domains, multipartite effectors, and fusion proteins have reduced potential for immunogenicity, supporting increased therapeutic potential and safety when used in therapeutic applications in human subjects. In addition, not only are the provided transcriptional activation domains, multipartite effectors, and fusion proteins less likely to be immunogenetic, as described herein, they have been observed to exhibit robust effector function (e.g., increased transcription at an exemplary target locus) that is similar to, or in some cases improved compared to available fusion proteins with transcriptional activator domains such as VP64. Further, without wishing to be bound by theory, the provided transcriptional activation domains, multipartite effectors, and fusion proteins also may have additional functions and modes of action that leads to more robust activity at the target locus, compared to available fusion proteins with transcriptional activator domains such as VP64.

In some aspects, the transcriptional activation domains and multipartite effectors for transcriptional activations, and fusion proteins comprising the transcriptional activation domains, provide for potentials for more robust activity and refined control of transcriptional activity at a target locus, for example as the transcriptional activation domains and multipartite effectors contain domains from proteins having various functional activities (for example, domains from proteins involved in signal transduction or other protein-protein interaction) and can recruit other molecules and machinery to the target site, compared to transcriptional activation domains that are known to be mainly involved in recruiting canonical transcriptional machinery. In some aspects, the expanded set of transcriptional activation domains may allow for increased control of the degree of transcriptional activation at a given locus. In some aspects, a transcriptional activation domains, fusion proteins, and multipartite activators provided herein may provide increased degree of transcriptional activation, or increased durability of transcriptional activation, when targeted to a target site. In some aspects, the increased degree or durability of transcriptional activation may increase the therapeutic effect of the targeted transcriptional activation, e.g. by reducing the need for repeated administration and/or by increasing the effect of administration.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Domains for Targeted Transcriptional Activation

In some aspects, provided herein are transcriptional activation domains. Also provided are fusion proteins, effector proteins and/or DNA-targeting systems that contain two or more of the transcriptional activation domains. In some aspects, provided herein are multipartite effectors for transcriptional activation, e.g., multipartite activators, comprising two or more transcriptional activation domains, such as any provided herein. In some aspects, the provided fusion proteins comprise one or more of the provided multipartite effectors. In some embodiments, the provided DNA-targeting systems comprise one or more of the provided multipartite effectors. In some aspects, the transcriptional activation domains and multipartite activators increase, or are capable of increasing, transcription of an endogenous locus when recruited to a target site at the endogenous locus, for example increasing transcription of a gene when recruited to a target site for the gene. In some aspects, the transcriptional activation domains and multipartite activators are provided as part of a DNA-targeting system or fusion protein, such as any described herein. In some aspects, the transcriptional activation domains and multipartite activators are targeted to one or more target sites for a gene (or multiple genes) to activate, induce, catalyze, or lead to increased transcription of the gene. In some aspects, the transcriptional activation domains and multipartite activators are targeted to the target site via a DNA-targeting domain, such as a CRISPR/Cas-based, ZFN-based, or TALE-based DNA-targeting domain, including any of the DNA-targeting domains described herein, for example, in Section III.

### A. Transcriptional Activation Domains

In some aspects, provided herein are transcriptional activation domains. In some aspects, a transcriptional activation domain increases transcription of an endogenous locus when recruited to a target site at the endogenous locus. In some embodiments, the transcriptional activation domain is a domain that induces, catalyzes, or leads to increased transcription of a gene when ectopically recruited to the gene or a DNA regulatory element thereof. In some embodiments, the transcriptional activation domain activates, induces, catalyzes, or leads to: transcription activation, transcription co-activation, transcription elongation, or transcription de-repression. In some embodiments, the transcriptional activation domain induces transcriptional activation. In some embodiments, the transcriptional activation domain has one of the aforementioned activities itself (i.e. acts directly). In some embodiments, the effector domain recruits and/or interacts with a polypeptide domain that has one of the aforementioned activities (i.e. acts indirectly).

Activation of gene expression of endogenous genes, such as human genes, can be achieved by targeting (e.g. via a CRISPR-based, ZFN-based, or TALE-based DNA-targeting domain) of transcriptional activation domains to a target site for the genes, such as regulatory DNA elements thereof (e.g. a promoter or enhancer).

In some embodiments, a transcriptional activation domain provided herein comprises a domain from a human protein. In some embodiments, a transcriptional activation domain from a protein comprises any portion of the protein that is capable of acting as a transcriptional activation domain as described herein. In some embodiments, a transcription activation domain is or comprises a portion, fragment, domain or variant of a human protein, such as a portion, fragment, domain or variant of a human protein selected from among DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, and NOTCH2, that exhibits transcriptional activation, is capable of inducing or activating transcription from a gene), is a functional transcriptional activation domain, and/or has a function of transcription activation. In some embodiments, a transcription activation domain is or comprises a functional portion, a functional fragment, a functional domain or a functional variant of a human protein, such as a portion, fragment, domain or variant of a human protein selected from among DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, and NOTCH2, that exhibits transcriptional activation, is capable of inducing or activating transcription from a gene), is a functional transcriptional activation domain, and/or has a function of transcription activation. In some embodiments, a transcription activation domain is or comprises a partially or fully functional portion, a partially or fully functional fragment, a partially or fully functional domain or a partially or fully functional variant of a human protein, such as a portion, fragment, domain or variant of a human protein selected from among DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, and NOTCH2, that exhibits increases the transcription from a gene by at least 5%, 10%, 20%, 30%, 40% or 50%, 60%, 70%, 80%, 85%, 90%, or 100% or more, such as 2-fold, 5-fold, 10-fold, 20-fold, 30-fold, 40-fold, 50-fold, 60-fold, 70-fold, 80-fold, 90-fold, 100-fold, 200-fold, 300-fold, 400-food, 500-fold, 1000-fold or more, compared to the absence of the transcriptional activation domain.

In some embodiments, the transcriptional activation domain is 10, 15, 20, 22, 25, 30, 35, 37, 40, 42, 45, 47, 49, 50, 55, 57, 60, 61, 62, 65, 70, 72, 75, 76, or 80 amino acids in length, or within a range defined by any of the foregoing. In some embodiments, the transcriptional activation domain is at least 10, 15, 20, 22, 25, 30, 35, 37, 40, 42, 45, 47, 49, 50, 55, 57, 60, 61, 62, 65, 70, 72, 75, 76, or 80 amino acids in length. In some embodiments, the transcriptional activation domain is 10, 15, 20, 25, 30, 35, 40, 45, 50, 55, 60, 65, 70, 75, or 80 amino acids in length, or within a range defined by any of the foregoing. In some embodiments, the transcriptional activation domain is at least 10, 15, 20, 25, 30, 35, 40, 45, 50, 55, 60, 65, 70, 75, or 80 amino acids in length, or within a range defined by any of the foregoing. In some embodiments, the transcriptional activation domain is 22, 37, 42, 47, 49, 57, 61, 62, 70, 72, 76, or 80 amino acids in length, or within a range defined by any of the foregoing. In some embodiments, the transcriptional activation domain is at least 22, 37, 42, 47, 49, 57, 61, 62, 70, 72, 76, or 80 amino acids in length. In some embodiments, the transcriptional activation domain is between 10 and 80, 20 and 70, 30 and 80, 30 and 70, 30 and 60, 40 and 80, 40 and 70, 40 and 60, 40 and 50, 50 and 80, 50 and 70, 50 and 60 amino acids in length.

In some embodiments, the transcriptional activation domain comprises a transcriptional activation domain described in WO 2021/226077.

In some embodiments, a transcriptional activation domain comprises a domain from DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, or NOTCH2. In some aspects, a domain from a gene is referred to as a gene domain. For example, a domain from DPOLA may be referred to as a DPOLA domain herein. In any of the provided embodiments, the domain from DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, or NOTCH2, is or comprises the respective transcriptional activation domains described herein or a partially or fully functional fragment thereof, a domain thereof, or a portion thereof, such as a contiguous portion thereof of at least 10, 15, 20, 25, 30, 35, 40, 45, 50, 55, 60, 65, 70, 75, or 80 amino acids, such as at least 20 amino acids, or a variant thereof. In any of the provided embodiments, the domain from DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, or NOTCH2, is or comprises the sequence of the respective transcriptional activation domains described herein or a partially or fully functional fragment thereof, a domain thereof, or a portion thereof, such as a contiguous portion thereof of at least 10, 15, 20, 25, 30, 35, 40, 45, 50, 55, 60, 65, 70, 75, or 80 amino acids, such as at least 20 amino acids, or a variant thereof.

In some embodiments, the transcriptional activation domain comprises or is selected from a transcriptional activation domain shown in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof or an amino acid sequence that has at least 90%, 91%, 92%, 93%, 94%, 95%, 96%, 97%, 98%, or 99% sequence identity to any of transcriptional activation domain shown in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain comprises or is selected from a transcriptional activation domain shown in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof.

In some embodiments, the transcriptional activation domain comprises or is selected from a transcriptional activation domain shown in Table 1, or a domain or a portion thereof, such as a contiguous portion thereof of 10, 15, 20, 22, 25, 30, 35, 37, 40, 42, 45, 47, 49, 50, 55, 57, 60, 61, 62, 65, 70, 72, 75, 76, or 80 amino acids in length, or within a range defined by any of the foregoing. In some embodiments, the transcriptional activation domain comprises or is selected from a transcriptional activation domain shown in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids in length. In some embodiments, the transcriptional activation domain comprises or is selected from a transcriptional activation domain shown in Table 1, or a domain or a portion thereof, such as a contiguous portion thereof of 10, 15, 20, 25, 30, 35, 40, 45, 50, 55, 60, 65, 70, 75, or 80 amino acids in length, or within a range defined by any of the foregoing. In some embodiments, the transcriptional activation domain comprises or is selected from a transcriptional activation domain shown in Table 1, or a domain or a portion thereof, such as a contiguous portion thereof of at least 10, 15, 20, 25, 30, 35, 40, 45, 50, 55, 60, 65, 70, 75, or 80 amino acids in length, or within a range defined by any of the foregoing. In some embodiments, the transcriptional activation domain comprises or is selected from a transcriptional activation domain shown in Table 1, or a domain or a portion thereof, such as a contiguous portion thereof of 22, 37, 42, 47, 49, 57, 61, 62, 70, 72, 76, or 80 amino acids in length, or within a range defined by any of the foregoing. In some embodiments, the transcriptional activation domain comprises or is selected from a transcriptional activation domain shown in Table 1, or a domain or a portion thereof, such as a contiguous portion thereof of at least 22, 37, 42, 47, 49, 57, 61, 62, 70, 72, 76, or 80 amino acids in length. In some embodiments, the transcriptional activation domain comprises or is selected from a transcriptional activation domain shown in Table 1, or a domain or a portion thereof, such as a contiguous portion thereof of between 10 and 80, 20 and 70, 30 and 80, 30 and 70, 30 and 60, 40 and 80, 40 and 70, 40 and 60, 40 and 50, 50 and 80, 50 and 70, 50 and 60 amino acids in length. In some embodiments, the transcriptional activation domain is a transcriptional activation domain set forth in Table 1. Table 1 shows a list of human genes and exemplary transcriptional activation domains from each gene.

In some embodiments, any of the provided multipartite effector proteins, fusion proteins, and/or DNA targeting systems, such as a multipartite activator, comprises a combination of transcriptional activation domains, such as a combination of two or more, such as three or more, such as three or more, of any of transcriptional activation domains shown in Table 1. In some embodiments, any of the provided multipartite effector proteins, fusion proteins, and/or DNA targeting systems, such as a multipartite activator, comprises a combination of two or more, such as three or more, of any one of the SEQ ID NOS set forth in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to any one of the SEQ ID NOS:set forth in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, any of the provided multipartite effector proteins, fusion proteins, and/or DNA targeting systems, such as a multipartite activator, comprises a combination of two or more, such as three or more, ofany one of the SEQ ID NOS:set forth in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, any of the provided multipartite effector proteins, fusion proteins, and/or DNA targeting systems, such as a multipartite activator, comprises two or more, such as three or more, of any one of the SEQ ID NOS: set forth in Table 1.

TABLE 1
Human proteins and transcriptional activation domains

| | | SEQ ID NO: | |
|---|---|---|---|
| | SEQ ID NO: | of 80 amino | SEQ ID NO: |
| | of full | acid | of alternative |
| | length | transcription | transcription |
| | human | activation | activation |
| Gene | protein | domain | domain |
| DPOLA | 35 | 22 | 176 |
| ENL | 36 | 23 | 131 |
| FOXO3 | 37 | 24 | 132 |
| HSH2D | 38 | 25 | 134 |
| NCOA2 | 39 | 26 | 135 |
| NCOA3 | 40 | 27 | 133 |
| PSA1 | 41 | 28 | 177 |
| PYGO1 | 42 | 29 | 130 |
| RBM39 | 43 | 30 | 178 |
| HERC2 | 44 | 31 | 179 |
| NOTCH2 | 46 or 390 | 33 or 381 | 136 |
| ZNF473 | 387 | 378 | |
| ANM2 | 388 | 379 | |
| KIBRA | 389 | 380 | |
| IKKA | 391 | 382 | |
| APBB1 | 392 | 383 | |
| SMN2 | 393 | 384 | |
| SERTAD2 | 394 | 385 | |
| MYBA | 395 | 386 | |

In some embodiments, the transcriptional activation domain comprises any one of SEQ ID NOS:35-44, 387-395, and 46, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 9900 sequence identity to any one of SEQ ID NOS:35-44, 387-395, and 46, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof.

In some embodiments, the transcriptional activation domain comprises any one of SEQ ID NOS:22-31, 33, 130-136, 378-386 and 176-179, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to any one of SEQ ID NOS:22-31, 33, 130-136, 378-386 and 176-179, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof.

In some embodiments, a transcriptional activation domain comprises a DPOLA domain, i.e. a domain from DPOLA. In some aspects, DPOLA refers to the DNA polymerase alpha catalytic subunit protein encoded by the human POLA1 gene. DPOLA plays an essential role in the initiation of DNA synthesis. An exemplary human DPOLA sequence is set forth in SEQ ID NO:35. An exemplary DPOLA domain sequence is set forth in SEQ ID NO:22 and SEQ ID NO:176. In some embodiments, the transcriptional activation domain comprises a sequence set forth in any of SEQ ID NOS:35, 22, and 176 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in any of SEQ ID NOS:35, 22, and 176 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:22. In some embodiments, the transcriptional activation domain comprises a contiguous portion of SEQ ID NO:35 that is at least 80 amino acids in length. In some embodiments, the transcriptional activation domain comprises SEQ ID NO:22. In some embodiments, the transcriptional activation domain is set forth in SEQ ID NO:22. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:176. In some embodiments, the transcriptional activation domain comprises a contiguous portion of SEQ ID NO:35 that is at least 61 amino acids in length. In some embodiments, the transcriptional activation domain comprises SEQ ID NO:176. In some embodiments, the transcriptional activation domain is set forth in SEQ ID NO:176.

In some embodiments, a transcriptional activation domain comprises a ENL domain, i.e. a domain from ENL. In some aspects, ENL refers to the ENL protein encoded by the human MLLT1 gene. ENL functions as a chromatin reader component of the super elongation complex (SEC), a complex which increases the catalytic rate of RNA polymerase II transcription. An exemplary human ENL sequence is set forth in SEQ ID NO:36. An exemplary ENL domain sequence is set forth in SEQ ID NO:23 and SEQ ID NO:131. In some embodiments, the transcriptional activation domain comprises a sequence set forth in any of SEQ ID NOS:36, 23, and 131 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in any of SEQ ID NOS:36, 23, and 131 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:23. In some embodiments, the transcriptional activation domain comprises a contiguous portion of SEQ ID NO:36 that is at least 80 amino acids in length. In some embodiments, the transcriptional activation domain comprises SEQ ID NO:23. In some embodiments, the transcriptional activation domain is set forth in SEQ ID NO:23. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:131. In some embodiments, the transcriptional activation domain comprises a contiguous portion of SEQ ID NO:36 that is at least 62 amino acids in length. In some embodiments, the transcriptional activation domain comprises SEQ ID NO:131. In some embodiments, the transcriptional activation domain is set forth in SEQ ID NO:131.

In some embodiments, a transcriptional activation domain comprises a FOXO3 domain, i.e. a domain from FOXO3. In some aspects, FOXO3 refers to the Forkhead box protein 03 encoded by the human FOXO3 gene. FOXO3 functions as a transcriptional activator that recognizes and binds to specific DNA sequences. An exemplary human FOXO3 sequence is set forth in SEQ ID NO:37. An exemplary FOXO3 domain sequence is set forth in SEQ ID NO:24 and SEQ ID NO:132. In some embodiments, the transcriptional activation domain comprises a sequence set forth in any of SEQ ID NOS:37, 24, and 132 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in any of SEQ ID NOS:37, 24, and 132 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:24. In some embodiments, the transcriptional activation domain comprises a contiguous portion of SEQ ID NO:37 that is at least 80 amino acids in length. In some embodiments, the transcriptional activation domain comprises SEQ ID NO:24. In some embodiments, the transcriptional activation domain is set forth in SEQ ID NO:24. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:132. In some embodiments, the transcriptional activation domain comprises a contiguous portion of SEQ ID NO:37 that is at least 42 amino acids in length. In some embodiments, the transcriptional activation domain comprises SEQ ID NO:132. In some embodiments, the transcriptional activation domain is set forth in SEQ ID NO:132.

In some embodiments, a transcriptional activation domain comprises a HSH2D domain, i.e. a domain from HSH2D. In some aspects, HSH2D refers to the Hematopoietic SH2 domain-containing protein encoded by the human HSH2D gene. HSH2D functions as an adapter protein involved in tyrosine kinase and CD28 signaling. An exemplary human HSH2D sequence is set forth in SEQ ID NO:38. An exemplary HSH2D domain sequence is set forth in SEQ ID NO:25 and SEQ ID NO:134. In some embodiments, the transcriptional activation domain comprises a sequence set forth in any of SEQ ID NOS:38, 25, and 134 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in any of SEQ ID NOS:38, 25, and 134 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:25. In some embodiments, the transcriptional activation domain comprises a contiguous portion of SEQ ID NO:38 that is at least 80 amino acids in length. In some embodiments, the transcriptional activation domain comprises SEQ ID NO:25. In some embodiments, the transcriptional activation domain is set forth in SEQ ID NO:25. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:134. In some embodiments, the transcriptional activation domain comprises a contiguous portion of SEQ ID NO:38 that is at least 76 amino acids in length. In some embodiments, the transcriptional activation domain comprises SEQ ID NO: 134. In some embodiments, the transcriptional activation domain is set forth in SEQ ID NO:134.

In some embodiments, a transcriptional activation domain comprises a NCOA2 domain, i.e. a domain from NCOA2. In some aspects, NCOA2 refers to the Nuclear receptor coactivator 2 protein encoded by the human NCOA2 gene. NCOA2 functions as a transcriptional coactivator for steroid receptors and nuclear receptors. An exemplary human NCOA2 sequence is set forth in SEQ ID NO:39. An exemplary NCOA2 domain sequence is set forth in SEQ ID NO:26 and SEQ ID NO:135. In some embodiments, the transcriptional activation domain comprises a sequence set forth in any of SEQ ID NOS:39, 26, and 135 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in any of SEQ ID NOS:39, 26, and 135 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:26. In some embodiments, the transcriptional activation domain comprises a contiguous portion of SEQ ID NO:39 that is at least 80 amino acids in length. In some embodiments, the transcriptional activation domain comprises SEQ ID NO:26. In some embodiments, the transcriptional activation domain is set forth in SEQ ID NO:26. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:135. In some embodiments, the transcriptional activation domain comprises a contiguous portion of SEQ ID NO:39 that is at least 47 amino acids in length. In some embodiments, the transcriptional activation domain comprises SEQ ID NO:135. In some embodiments, the transcriptional activation domain is set forth in SEQ ID NO:135.

In some embodiments, a transcriptional activation domain comprises a NCOA3 domain, i.e. a domain from NCOA3. In some aspects, NCOA3 refers to the Nuclear receptor coactivator 3 protein encoded by the human NCOA3 gene. NCOA3 functions as a transcriptional coactivator for steroid receptors and nuclear receptors. An exemplary human NCOA3 sequence is set forth in SEQ ID NO:40. An exemplary NCOA3 domain sequence is set forth in SEQ ID NO:27 and SEQ ID NO:133. In some embodiments, the transcriptional activation domain comprises a sequence set forth in any of SEQ ID NOS:40, 27, and 133 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in any of SEQ ID NOS:40, 27, and 133 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:27. In some embodiments, the transcriptional activation domain comprises a contiguous portion of SEQ ID NO:40 that is at least 80 amino acids in length. In some embodiments, the transcriptional activation domain comprises SEQ ID NO:27. In some embodiments, the transcriptional activation domain is set forth in SEQ ID NO:27. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:133. In some embodiments, the transcriptional activation domain comprises a contiguous portion of SEQ ID NO:40 that is at least 49 amino acids in length. In some embodiments, the transcriptional activation domain comprises SEQ ID NO:133. In some embodiments, the transcriptional activation domain is set forth in SEQ ID NO:133.

In some embodiments, a transcriptional activation domain comprises a PSA1 domain, i.e. a domain from PSA1. In some aspects, PSA1 refers to the Proteasome subunit alpha type-1 protein encoded by the human PSMA1 gene. PSA1 functions as a component of the 20S core proteasome complex, which facilitates proteolytic degradation of intracellular proteins. An exemplary human PSA1 sequence is set forth in SEQ ID NO:41. An exemplary PSA1 domain sequence is set forth in SEQ ID NO:28 and SEQ ID NO:177. In some embodiments, the transcriptional activation domain comprises a sequence set forth in any of SEQ ID NOS:41, 28, and 177 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in any of SEQ ID NOS:41, 28, and 177 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:28. In some embodiments, the transcriptional activation domain comprises a contiguous portion of SEQ ID NO:41 that is at least 80 amino acids in length. In some embodiments, the transcriptional activation domain comprises SEQ ID NO:28. In some embodiments, the transcriptional activation domain is set forth in SEQ ID NO:28. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:177. In some embodiments, the transcriptional activation domain comprises a contiguous portion of SEQ ID NO:41 that is at least 22 amino acids in length. In some embodiments, the transcriptional activation domain comprises SEQ ID NO:177. In some embodiments, the transcriptional activation domain is set forth in SEQ ID NO:177.

In some embodiments, a transcriptional activation domain comprises a PYGO1 domain, i.e. a domain from PYGO1. In some aspects, PYGO1 refers to the Pygopus homolog 1 protein encoded by the human PYGO1 gene. PYGO1 is involved in Wnt pathway signal transduction. An exemplary human PYGO1 sequence is set forth in SEQ ID NO:42. An exemplary PYGO1 domain sequence is set forth in SEQ ID NO:29 and SEQ ID NO:130. In some embodiments, the transcriptional activation domain comprises a sequence set forth in any of SEQ ID NOS:42, 29, and 130 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in any of SEQ ID NOS:42, 29, and 130 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:29. In some embodiments, the transcriptional activation domain comprises a contiguous portion of SEQ ID NO:42 that is at least 80 amino acids in length. In some embodiments, the transcriptional activation domain comprises SEQ ID NO:29. In some embodiments, the transcriptional activation domain is set forth in SEQ ID NO:29. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:130. In some embodiments, the transcriptional activation domain comprises a contiguous portion of SEQ ID NO:42 that is at least 57 amino acids in length. In some embodiments, the transcriptional activation domain comprises SEQ ID NO:130. In some embodiments, the transcriptional activation domain is set forth in SEQ ID NO:130.

In some embodiments, a transcriptional activation domain comprises a RBM39 domain, i.e. a domain from RBM39. In some aspects, RBM39 refers to the RNA-binding protein 39 protein encoded by the human RBM39 gene. RBM39 functions as a RNA-binding protein that acts as a pre-mRNA splicing factor. An exemplary human RBM39 sequence is set forth in SEQ ID NO:43. An exemplary RBM39 domain sequence is set forth in SEQ ID NO:30 and SEQ ID NO:178. In some embodiments, the transcriptional activation domain comprises a sequence set forth in any of SEQ ID NOS:43, 30, and 178 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in any of SEQ ID NOS:43, 30, and 178 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:30. In some embodiments, the transcriptional activation domain comprises a contiguous portion of SEQ ID NO:43 that is at least 80 amino acids in length. In some embodiments, the transcriptional activation domain comprises SEQ ID NO:30. In some embodiments, the transcriptional activation domain is set forth in SEQ ID NO:30. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:178. In some embodiments, the transcriptional activation domain comprises a contiguous portion of SEQ ID NO:43 that is at least 70 amino acids in length. In some embodiments, the transcriptional activation domain comprises SEQ ID NO:178. In some embodiments, the transcriptional activation domain is set forth in SEQ ID NO:178.

In some embodiments, a transcriptional activation domain comprises a HERC2 domain, i.e. a domain from HERC2. In some aspects, HERC2 refers to the E3 ubiquitin-protein ligase HERC2 protein encoded by the human HERC2 gene. HERC2 functions as a regulator of ubiquitin-dependent retention of repair proteins on damaged chromosomes. An exemplary human HERC2 sequence is set forth in SEQ ID NO:44. An exemplary HERC2 domain sequence is set forth in SEQ ID NO:31 and SEQ ID NO: 179. In some embodiments, the transcriptional activation domain comprises a sequence set forth in any of SEQ ID NOS:44, 31, and 179 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in any of SEQ ID NOS:44, 31, and 179 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:31. In some embodiments, the transcriptional activation domain comprises a contiguous portion of SEQ ID NO:44 that is at least 80 amino acids in length. In some embodiments, the transcriptional activation domain comprises SEQ ID NO:31. In some embodiments, the transcriptional activation domain is set forth in SEQ ID NO:31. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:179. In some embodiments, the transcriptional activation domain comprises a contiguous portion of SEQ ID NO:44 that is at least 72 amino acids in length. In some embodiments, the transcriptional activation domain comprises SEQ ID NO:179. In some embodiments, the transcriptional activation domain is set forth in SEQ ID NO:179.

In some embodiments, a transcriptional activation domain comprises a NOTCH2 domain, i.e. a domain from NOTCH2. In some aspects, NOTCH2 refers to the Neurogenic locus notch homolog protein 2 protein encoded by the human NOTCH2 gene. NOTCH2 functions as a receptor for membrane-bound ligands such as Delta-1 to regulate cell-fate determination. An exemplary human NOTCH2 sequence is set forth in SEQ ID NO:46 and SEQ ID NO:390. An exemplary NOTCH2 domain sequence is set forth in SEQ ID NO:33, SEQ ID NO:381, and SEQ ID NO:136. In some embodiments, the transcriptional activation domain comprises a sequence set forth in any of SEQ ID NOS:46, 390, 381, 33, and 136 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in any of SEQ ID NOS:46, 390, 381, 33, and 136 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:33 or SEQ ID NO:381. In some embodiments, the transcriptional activation domain comprises a contiguous portion of SEQ ID NO:46 or 390 that is at least 80 amino acids in length. In some embodiments, the transcriptional activation domain comprises SEQ ID NO:33. In some embodiments, the transcriptional activation domain is set forth in SEQ ID NO:33. In some embodiments, the transcriptional activation domain comprises SEQ ID NO:381. In some embodiments, the transcriptional activation domain is set forth in SEQ ID NO:381. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:136. In some embodiments, the transcriptional activation domain comprises a contiguous portion of SEQ ID NO:46 that is at least 37 amino acids in length. In some embodiments, the transcriptional activation domain comprises SEQ ID NO:136. In some embodiments, the transcriptional activation domain is set forth in SEQ ID NO:136.

In some embodiments, a transcriptional activation domain comprises a ZNF473 domain, i.e. a domain from ZNF473. In some aspects, ZNF473 refers to the Zinc finger protein 473 protein encoded by the human ZNF473 gene. An exemplary human ZNF473 sequence is set forth in SEQ ID NO:387. An exemplary ZNF473 domain sequence is set forth in SEQ ID NO:378. In some embodiments, the transcriptional activation domain comprises a sequence set forth in SEQ ID NO:387 or 378, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in SEQ ID NO:387 or 378 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:378. In some embodiments, the transcriptional activation domain comprises a contiguous portion of SEQ ID NO:387 that is at least 80 amino acids in length. In some embodiments, the transcriptional activation domain comprises SEQ ID NO:378. In some embodiments, the transcriptional activation domain is set forth in SEQ ID NO:378.

In some embodiments, a transcriptional activation domain comprises a ANM2 domain, i.e. a domain from ANM2. In some aspects, ANM2 refers to the Protein arginine N-methyltransferase 2 protein encoded by the human PRMT2 gene. An exemplary human ANM2 sequence is set forth in SEQ ID NO:388. An exemplary ANM2 domain sequence is set forth in SEQ ID NO:379. In some embodiments, the transcriptional activation domain comprises a sequence set forth in SEQ ID NO:388 or 379, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in SEQ ID NO:388 or 379 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:379. In some embodiments, the transcriptional activation domain comprises a contiguous portion of SEQ ID NO:388 that is at least 80 amino acids in length. In some embodiments, the transcriptional activation domain comprises SEQ ID NO:379. In some embodiments, the transcriptional activation domain is set forth in SEQ ID NO:379.

In some embodiments, a transcriptional activation domain comprises a KIBRA domain, i.e. a domain from KIBRA. In some aspects, KIBRA refers to the KIBRA protein encoded by the human WWC1 gene. An exemplary human KIBRA sequence is set forth in SEQ ID NO:389. An exemplary KIBRA domain sequence is set forth in SEQ ID NO:380. In some embodiments, the transcriptional activation domain comprises a sequence set forth in SEQ ID NO:389 or 380, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in SEQ ID NO:389 or 380 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:380. In some embodiments, the transcriptional activation domain comprises a contiguous portion of SEQ ID NO:389 that is at least 80 amino acids in length. In some embodiments, the transcriptional activation domain comprises SEQ ID NO:380. In some embodiments, the transcriptional activation domain is set forth in SEQ ID NO:380.

In some embodiments, a transcriptional activation domain comprises a IKKA domain, i.e. a domain from IKKA. In some aspects, IKKA refers to the Inhibitor of nuclear factor kappa-B kinase subunit alpha protein encoded by the human CHUK gene. An exemplary human IKKA sequence is set forth in SEQ ID NO:391. An exemplary IKKA domain sequence is set forth in SEQ ID NO:382. In some embodiments, the transcriptional activation domain comprises a sequence set forth in SEQ ID NO:391 or 382, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in SEQ ID NO:391 or 382 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:382. In some embodiments, the transcriptional activation domain comprises a contiguous portion of SEQ ID NO:391 that is at least 80 amino acids in length. In some embodiments, the transcriptional activation domain comprises SEQ ID NO:382. In some embodiments, the transcriptional activation domain is set forth in SEQ ID NO:382.

In some embodiments, a transcriptional activation domain comprises a APBB1 domain, i.e. a domain from APBB1. In some aspects, APBB1 refers to the Amyloid beta precursor protein binding family B member 1 protein encoded by the human APBB1 gene. An exemplary human APBB1 sequence is set forth in SEQ ID NO:392. An exemplary APBB1 domain sequence is set forth in SEQ ID NO:383. In some embodiments, the transcriptional activation domain comprises a sequence set forth in SEQ ID NO:392 or 383, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in SEQ ID NO:392 or 383 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:383. In some embodiments, the transcriptional activation domain comprises a contiguous portion of SEQ ID NO:392 that is at least 80 amino acids in length. In some embodiments, the transcriptional activation domain comprises SEQ ID NO:383. In some embodiments, the transcriptional activation domain is set forth in SEQ ID NO:383.

In some embodiments, a transcriptional activation domain comprises a SMN2 domain, i.e. a domain from SMN2. In some aspects, SMN2 refers to the Survival motor neuron protein encoded by the human SMN1 or SMN2 gene. An exemplary human SMN2 sequence is set forth in SEQ ID NO:393. An exemplary SMN2 domain sequence is set forth in SEQ ID NO:384. In some embodiments, the transcriptional activation domain comprises a sequence set forth in SEQ ID NO:393 or 384, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in SEQ ID NO:393 or 384 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:384. In some embodiments, the transcriptional activation domain comprises a contiguous portion of SEQ ID NO:393 that is at least 80 amino acids in length. In some embodiments, the transcriptional activation domain comprises SEQ ID NO:384. In some embodiments, the transcriptional activation domain is set forth in SEQ ID NO:384.

In some embodiments, a transcriptional activation domain comprises a SERTAD2 domain, i.e. a domain from SERTAD2. In some aspects, SERTAD2 refers to the SERTA domain-containing protein 2 protein encoded by the human SERTAD2 gene. An exemplary human SERTAD2 sequence is set forth in SEQ ID NO:394. An exemplary SERTAD2 domain sequence is set forth in SEQ ID NO:385. In some embodiments, the transcriptional activation domain comprises a sequence set forth in SEQ ID NO:394 or 385, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in SEQ ID NO:394 or 385 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:385. In some embodiments, the transcriptional activation domain comprises a contiguous portion of SEQ ID NO:394 that is at least 80 amino acids in length. In some embodiments, the transcriptional activation domain comprises SEQ ID NO:385. In some embodiments, the transcriptional activation domain is set forth in SEQ ID NO:385.

In some embodiments, a transcriptional activation domain comprises a MYBA domain, i.e. a domain from MYBA. In some aspects, MYBA refers to the Myb-related protein A protein encoded by the human MYBA gene. An exemplary human MYBA sequence is set forth in SEQ ID NO:395. An exemplary MYBA domain sequence is set forth in SEQ ID NO:386. In some embodiments, the transcriptional activation domain comprises a sequence set forth in SEQ ID NO:395 or 386, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to a sequence set forth in SEQ ID NO:395 or 386 or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, the transcriptional activation domain is or comprises an amino acid sequence that has at least 90%, 91%, 92%, 93%, 94%, 95%, 96%, 97%, 98%, or 99% sequence identity to SEQ ID NO:386. In some embodiments, the transcriptional activation domain comprises a contiguous portion of SEQ ID NO:395 that is at least 80 amino acids in length. In some embodiments, the transcriptional activation domain comprises SEQ ID NO:386. In some embodiments, the transcriptional activation domain is set forth in SEQ ID NO:386.

A variety of other effector domains for transcriptional activation (e.g. transcriptional activation domains) are known and can be used in accord with or in conjunction with the provided embodiments. Other transcriptional activation domains for targeted activation are described, for example, in WO 2014/197748, WO 2016/130600, WO 2017/180915, WO 2021/226555, WO 2021/226077, WO 2013/176772, WO 2014/152432, WO 2014/093661, WO 2021/247570, Adli, M. Nat. Commun. 9, 1911 (2018), Perez-Pinera, P. et al. Nat. Methods 10, 973-976 (2013), Mali, P. et al. Nat. Biotechnol. 31, 833-838 (2013), Maeder, M. L. et al. Nat. Methods 10, 977-979 (2013), Gilbert, L. A. et al. Cell 154(2):442-451 (2013), and Nunez, J. K. et al. Cell 184(9):2503-2519 (2021).

In some embodiments, a transcriptional activation domain comprises a domain of a protein selected from among VP64, p65, Rta, p300, CBP, VPR, VPH, HSF1, a TET protein (e.g. TET1), a partially or fully functional fragment or domain thereof, or a combination of any of the foregoing.

In some embodiments, the transcriptional activation domain comprises a VP64 domain. For example, dCas9-VP64 can be targeted to a target site by one or more gRNAs to activate a gene. VP64 is a polypeptide composed of four tandem copies of VP16, a 16 amino acid transactivation domain of the Herpes simplex virus. VP64 domains, including in dCas fusion proteins, have been described, for example, in WO 2014/197748, WO 2013/176772, WO 2014/152432, and WO 2014/093661. In some embodiments, the transcriptional activation domain comprises at least one VP16 domain, or a VP16 tetramer (“VP64”) or a variant thereof. An exemplary VP64 domain is set forth in SEQ ID NO:162. In some embodiments, the transcriptional activation domain comprises SEQ ID NO:162, or a portion thereof, or an amino acid sequence that has at least 90%, 91%, 92%, 93%, 94%, 95%, 96%, 97%, 98%, or 99% sequence identity to SEQ ID NO:162, or a portion thereof. In some embodiments, the transcriptional activation domain is set forth in SEQ ID NO:162.

In some embodiments, the transcriptional activation domain comprises a p65 activation domain (p65AD). p65AD is the principal transactivation domain of the 65 kDa polypeptide of the nuclear form of the NF-KB transcription factor. An exemplary sequence of human transcription factor p65 is available at the Uniprot database under accession number Q04206. p65 domains, including in dCas fusion proteins, have been described, for example in WO 2017/180915 and Chavez, A. et al. Nat. Methods 12, 326-328 (2015). An exemplary p65 activation domain is set forth in SEQ ID NO: 193. In some embodiments, the transcriptional activation domain comprises SEQ ID NO: 193, or a portion thereof, or an amino acid sequence that has at least 90%, 91%, 92%, 93%, 94%, 95%, 96%, 97%, 98%, or 99% sequence identity to SEQ ID NO:193, or a portion thereof. In some embodiments, the transcriptional activation domain is set forth in SEQ ID NO:193.

In some embodiments, the transcriptional activation domain comprises an R transactivator (Rta) domain. Rta is an immediate-early protein of Epstein-Barr virus (EBV), and is a transcriptional activator that induces lytic gene expression and triggers virus reactivation. The Rta domain, including in dCas fusion proteins, has been described, for example in WO 2017/180915 and Chavez, A. et al. Nat. Methods 12, 326-328 (2015). An exemplary Rta domain is set forth in SEQ ID NO:194. In some embodiments, the transcriptional activation domain comprises SEQ ID NO:194, or a portion thereof, or an amino acid sequence that has at least 90%, 91%, 92%, 93%, 94%, 95%, 96%, 97%, 98%, or 99% sequence identity to SEQ ID NO:194, or a portion thereof. In some embodiments, the transcriptional activation domain is set forth in SEQ ID NO:194.

The transcriptional activation domain comprises a CREB-binding protein (CBP) domain or a p300 domain. In some aspects, CBP refers to the CREB-binding protein encoded by the human CREBBP gene. CBP is a coactivator that interacts with cAMP-response element binding protein (CREB). In some aspects, p300 refers to the Histone acetyltransferase p300 protein encoded by the human EP300 gene, and is a coactivator closely related to CBP. CBP and p300 each interact with a variety of transcriptional activators to affect gene transcription (Gerritsen, M. E. et al. PNAS 94(7):2927-2932 (1997)). In some embodiments, the transcriptional activation domain comprises a p300 domain. p300 domains (such as the catalytic core of p300) including in dCas fusion proteins for gene activation, has been described, for example, in WO 2016/130600, WO 2017/180915, and Hilton, I. B. et al., Nat. Biotechnol. 33(5):510-517 (2015). An exemplary human CBP sequence is set forth in SEQ ID NO:199. An exemplary human p300 sequence is set forth in SEQ ID NO:200. An exemplary p300 domain is set forth in SEQ ID NO:201. In some embodiments, the transcriptional activation domain comprises any one of SEQ ID NOS:199-201, or a portion thereof, or an amino acid sequence that has at least 90%, 91%, 92%, 93%, 94%, 95%, 96%, 97%, 98%, or 99% sequence identity to any one of SEQ ID NOS:199-201, or a portion thereof. In some embodiments, the transcriptional activation domain comprises SEQ ID NO:201, or a portion thereof, or an amino acid sequence that has at least 90%, 91%, 92%, 93%, 94%, 95%, 96%, 97%, 98%, or 99% sequence identity to SEQ ID NO:201, or a portion thereof. In some embodiments, the transcriptional activation domain is set forth in SEQ ID NO:201.

In some embodiments, the transcriptional activation domain comprises a HSF1 domain. In some aspects, HSF1 refers to the Heat shock factor protein 1 protein encoded by the human HSF1 gene. HSF1, including in dCas fusion proteins for gene activation, has been described, for example, in WO 2021/226555, WO 2015/089427, and Konermann et al. Nature 517(7536):583-8 (2015). An exemplary human HSF1 sequence is set forth in SEQ ID NO:202. An exemplary HSF1 domain sequence is set forth in SEQ ID NO:195. In some embodiments, the transcriptional activation domain comprises SEQ ID NO:195 or SEQ ID NO:202, or a portion thereof, or an amino acid sequence that has at least 90%, 91%, 92%, 93%, 94%, 95%, 96%, 97%, 98%, or 99% sequence identity to SEQ ID NO:195 or SEQ ID NO:202, or a portion thereof. In some embodiments, the transcriptional activation domain comprises SEQ ID NO:202, or a portion thereof, or an amino acid sequence that has at least 90%, 91%, 92%, 93%, 94%, 95%, 96%, 97%, 98%, or 99% sequence identity to SEQ ID NO:202, or a portion thereof. In some embodiments, the transcriptional activation domain is set forth in SEQ ID NO:202.

In some embodiments, the transcriptional activation domain comprises the tripartite activator VP64-p65-Rta (also known as VPR). VPR comprises three transcription activation domains (VP64, p65, and Rta) fused by short amino acid linkers, and can effectively upregulate target gene expression. VPR, including in dCas fusion proteins for gene activation, has been described, for example, in WO 2021/226555 and Chavez, A. et al. Nat. Methods 12, 326-328 (2015). An exemplary VPR polypeptide is set forth in SEQ ID NO:196. In some embodiments, the transcriptional activation domain comprises SEQ ID NO:196, or a portion thereof, or an amino acid sequence that has at least 90%, 91%, 92%, 93%, 94%, 95%, 96%, 97%, 98%, or 99% sequence identity to SEQ ID NO:196, or a portion thereof. In some embodiments, the transcriptional activation domain is set forth in SEQ ID NO:196.

In some embodiments, the transcriptional activation domain comprises VPH. VPH is a tripartite activator polypeptide comprising VP64, mouse p65, and HSF1. VPH, including in dCas fusion proteins for gene activation, has been described, for example, in WO 2021/226555. An exemplary VPH polypeptide is set forth in SEQ ID NO:197. In some embodiments, the transcriptional activation domain comprises SEQ ID NO:197, or a portion thereof, or an amino acid sequence that has at least 90%, 91%, 92%, 93%, 94%, 95%, 96%, 97%, 98%, or 99% sequence identity to SEQ ID NO:197, or a portion thereof. In some embodiments, the transcriptional activation domain is set forth in SEQ ID NO:197.

In some embodiments, the transcriptional activation domain has demethylase activity. The transcriptional activation domain can include an enzyme that removes methyl (CH3-) groups from nucleic acids, proteins (in particular histones), and other molecules. Alternatively, the transcriptional activation domain can convert the methyl group to hydroxymethylcytosine in a mechanism for demethylating DNA. The effector domain can catalyze this reaction. For example, the transcriptional activation domain that catalyzes this reaction may comprise a domain from a TET protein, for example TET1 (Ten-eleven translocation methylcytosine dioxygenase 1). In some aspects, TET1 refers to the Methylcytosine dioxygenase TET1 protein encoded by the human TET1 gene. TET1 catalyzes the conversion of the modified genomic base 5-methylcytosine (5mC) into 5-hydroxymethylcytosine (5hmC) and plays a key role in active DNA demethylation. TET1, including in dCas fusion proteins for gene activation, has been described, for example, in WO 2021/226555. An exemplary human TET1 sequence is set forth in SEQ ID NO:203. An exemplary TET1 catalytic domain is set forth in SEQ ID NO:198. In some embodiments, the transcriptional activation domain comprises SEQ ID NO:203 or SEQ ID NO:198, or a portion thereof, or an amino acid sequence that has at least 90%, 91%, 92%, 93%, 94%, 95%, 96%, 97%, 98%, or 99% sequence identity to SEQ ID NO:203 or SEQ ID NO:198, or a portion thereof. In some embodiments, the transcriptional activation domain comprises SEQ ID NO:198, or a portion thereof, or an amino acid sequence that has at least 90%, 91%, 92%, 93%, 94%, 95%, 96%, 97%, 98%, or 99% sequence identity to SEQ ID NO:198, or a portion thereof. In some embodiments, the transcriptional activation domain is set forth in SEQ ID NO: 198.

### B. Multipartite Effectors for Transcriptional Activation

In some aspects, provided herein are multipartite effectors for transcriptional activation, for example, multipartite transcriptional activation domains or multipartite activators. In some aspects, the multipartite activator is a fusion protein or a sequence of amino acids comprising two or more transcriptional activation domains, such as any of the transcriptional activation domains provided herein. In some aspects, the multipartite activator comprises two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. In some aspects, the multipartite activator is provided as part of a DNA-targeting system or fusion protein, such as any described herein.

In some aspects, the multipartite activator increases transcription of an endogenous locus when recruited to a target site at the endogenous locus. For example, the multipartite activator increases transcription of a gene when recruited (e.g. targeted to) a target site for the gene, such as a regulatory DNA element (e.g. a promoter or enhancer). Thus, in some aspects, a multipartite activator may itself be referred to as a transcriptional activation domain herein. In some embodiments, the multipartite activator induces, catalyzes, or leads to increased transcription of a gene when ectopically recruited to the gene or a DNA regulatory element thereof. In some embodiments, the multipartite activator activates, induces, catalyzes, or leads to: transcription activation, transcription co-activation, transcription elongation, or transcription de-repression. In some embodiments, the multipartite activator induces transcriptional activation. In some embodiments, the multipartite activator has one of the aforementioned activities itself (i.e. acts directly). In some embodiments, the multipartite activator recruits and/or interacts with a polypeptide domain that has one of the aforementioned activities (i.e. acts indirectly).

In some aspects, a multipartite activator provided herein comprises two or more transcriptional activation domains. In some aspects, the multipartite activator has an effect that is different from any one of the individual transcriptional activation domains comprised by the multipartite activator alone. The different effect may be quantitatively or qualitatively different. The multipartite activator may induce greater, more reliable, or more durable transcriptional activation of a target gene, in comparison to a transcriptional activation domain alone. The effect may be context-specific. For example, a multipartite activator may induce transcriptional activation in a specific context in which the transcriptional activation domain alone does not induce transcriptional activation to the same degree, at a detectable level, or at all, such as when targeted to a specific gene or target site of the gene. Thus, a multipartite activator does not necessarily lead to greater activation of a target gene than a transcriptional activation domain alone in every context, but may allow for activation of a target gene in different contexts and to a different degree than the transcriptional activation domain. A multipartite activator may have a more durable effect on transcription than a transcriptional activation domain alone. For example, a multipartite activator may lead to increased transcription of a target gene in a cell for a longer amount of time, or for a greater number of cell divisions or cell passages.

In some embodiments, the multipartite effector, e.g., multipartite activator, is a bipartite effector, e.g., bipartite activator (i.e. comprising two transcriptional activation domains). In some embodiments, the multipartite effector, e.g., multipartite activator, is a tripartite effector, e.g., tripartite activator (i.e. comprising three transcriptional activation domains). In some embodiments, the multipartite effector, e.g., multipartite activator comprises 4, 5, 6, 7, 8, 9, 10, or more transcriptional activation domains. In some embodiments, any two or more of the transcriptional activation domains are the same. In some embodiments, any two or more of the transcriptional activation domains are different.

In some embodiments, the multipartite activator comprises two or more transcriptional activation domains, each transcriptional activation domain comprising a domain of a protein selected from among DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, or NOTCH2. In some embodiments, the multipartite activator comprises two or more transcriptional activation domains, wherein one or more of the transcriptional activation domains comprises a domain of a protein selected from among DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, or NOTCH2. In some aspects, the transcriptional activation domain from DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, or NOTCH2, is or comprises any of the respective transcriptional activation domains described herein, for example, in Section IA, or a partially or fully functional fragment thereof, a domain thereof, or a portion thereof, such as a contiguous portion thereof of at least 30 amino acids, or a variant thereof. In some aspects, the transcriptional activation domain from DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, or NOTCH2, is or comprises any of sequences of the respective transcriptional activation domains described herein, for example, in Section IA, or a partially or fully functional fragment thereof, a domain thereof, or a portion thereof, such as a contiguous portion thereof of at least 30 amino acids, or a variant thereof.

In some embodiments, the multipartite activator further comprises one or more of any of the transcriptional activation domains provided herein, including any of the transcriptional activation domains described in Section I.A., such as VP64, p65, Rta, p300, CBP, VPR, VPH, HSF1, a TET protein (e.g. TET1), a partially or fully functional fragment or domain thereof, or a combination of any of the foregoing.

In some embodiments, the multipartite activator is a bipartite activator comprising a first transcriptional activation domain and a second transcriptional activation domain. In some aspects, each of the first transcriptional activation domain and the second transcriptional activation domain independently comprises a domain of a protein selected from among DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, and NOTCH2. In some aspects, each of the first transcriptional activation domain and the second transcriptional activation domain independently comprises a domain of a protein selected from among DPOLA, ENL, FOXO3, HSH2D, NCOA2, NCOA3, PSA1, PYGO1, RBM39, HERC2, and NOTCH2. In some embodiments, the first and second transcriptional activation domains, respectively, are from DPOLA and DPOLA; DPOLA and ENL; DPOLA and FOXO3; DPOLA and HERC2; DPOLA and HSH2D; DPOLA and NCOA2; DPOLA and NCOA3; DPOLA and NOTCH2; DPOLA and PSA1; DPOLA and PYGO1; DPOLA and RBM39; ENL and DPOLA; ENL and ENL; ENL and FOXO3; ENL and HERC2; ENL and HSH2D; ENL and NCOA2; ENL and NCOA3; ENL and NOTCH2; ENL and PSA1; ENL and PYGO1; ENL and RBM39; FOXO3 and DPOLA; FOXO3 and ENL; FOXO3 and FOXO3; FOXO3 and HERC2; FOXO3 and HSH2D; FOXO3 and NCOA2; FOXO3 and NCOA3; FOXO3 and NOTCH2; FOXO3 and PSA1; FOXO3 and PYGO1; FOXO3 and RBM39; HERC2 and DPOLA; HERC2 and ENL; HERC2 and FOXO3; HERC2 and HERC2; HERC2 and HSH2D; HERC2 and NCOA2; HERC2 and NCOA3; HERC2 and NOTCH2; HERC2 and PSA1; HERC2 and PYGO1; HERC2 and RBM39; HSH2D and DPOLA; HSH2D and ENL; HSH2D and FOXO3; HSH2D and HERC2; HSH2D and HSH2D; HSH2D and NCOA2; HSH2D and NCOA3; HSH2D and NOTCH2; HSH2D and PSA1; HSH2D and PYGO1; HSH2D and RBM39; NCOA2 and DPOLA; NCOA2 and ENL; NCOA2 and FOXO3; NCOA2 and HERC2; NCOA2 and HSH2D; NCOA2 and NCOA2; NCOA2 and NCOA3; NCOA2 and NOTCH2; NCOA2 and PSA1; NCOA2 and PYGO1; NCOA2 and RBM39; NCOA3 and DPOLA; NCOA3 and ENL; NCOA3 and FOXO3; NCOA3 and HERC2; NCOA3 and HSH2D; NCOA3 and NCOA2; NCOA3 and NCOA3; NCOA3 and NOTCH2; NCOA3 and PSA1; NCOA3 and PYGO1; NCOA3 and RBM39; NOTCH2 and DPOLA; NOTCH2 and ENL; NOTCH2 and FOXO3; NOTCH2 and HERC2; NOTCH2 and HSH2D; NOTCH2 and NCOA2; NOTCH2 and NCOA3; NOTCH2 and NOTCH2; NOTCH2 and PSA1; NOTCH2 and PYGO1; NOTCH2 and RBM39; PSA1 and DPOLA; PSA1 and ENL; PSA1 and FOXO3; PSA1 and HERC2; PSA1 and HSH2D; PSA1 and NCOA2; PSA1 and NCOA3; PSA1 and NOTCH2; PSA1 and PSA1; PSA1 and PYGO1; PSA1 and RBM39; PYGO1 and DPOLA; PYGO1 and ENL; PYGO1 and FOXO3; PYGO1 and HERC2; PYGO1 and HSH2D; PYGO1 and NCOA2; PYGO1 and NCOA3; PYGO1 and NOTCH2; PYGO1 and PSA1; PYGO1 and PYGO1; PYGO1 and RBM39; RBM39 and DPOLA; RBM39 and ENL; RBM39 and FOXO3; RBM39 and HERC2; RBM39 and HSH2D; RBM39 and NCOA2; RBM39 and NCOA3; RBM39 and NOTCH2; RBM39 and PSA1; RBM39 and PYGO1; or RBM39 and RBM39, respectively.

In some embodiments, the multipartite activator is a tripartite activator comprising a first transcriptional activation domain, a second transcriptional activation domain, and a third transcriptional activation domain. In some aspects, the first transcriptional activation domain, the second transcriptional activation domain, and the third transcriptional activation domain each independently comprises a domain of a protein selected from among DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, and NOTCH2. In some embodiments, the first and second transcriptional domains are the first and second transcriptional domains from any of the bipartite activators described above, and the third transcriptional domain independently comprises a domain of a protein selected from among DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, and NOTCH2.

In some embodiments, the multipartite activator is a tripartite activator comprising a first transcriptional activation domain, a second transcriptional activation domain, and a third transcriptional activation domain, each independently comprising a domain of a protein selected from among DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, and NOTCH2. In some embodiments, the multipartite activator is a tripartite activator comprising a first transcriptional activation domain, a second transcriptional activation domain, and a third transcriptional activation domain, each independently comprising a domain of a protein selected from among DPOLA, ENL, FOXO3, HSH2D, NCOA2, NCOA3, PSA1, PYGO1, RBM39, HERC2, and NOTCH2. In some embodiments, the first, second, and third transcriptional activation domains, respectively, are from NCOA3, NCOA3, and NCOA3; NCOA3, NCOA3, and ENL; NCOA3, NCOA3, and FOXO3; NCOA3, NCOA3, and PYGO1; NCOA3, NCOA3, and HSH2D; NCOA3, NCOA3, and NCOA2; NCOA3, NCOA3, and NOTCH2; NCOA3, ENL, and NCOA3; NCOA3, ENL, and ENL; NCOA3, ENL, and FOXO3; NCOA3, ENL, and PYGO1; NCOA3, ENL, and HSH2D; NCOA3, ENL, and NCOA2; NCOA3, ENL, and NOTCH2; NCOA3, FOXO3, and NCOA3; NCOA3, FOXO3, and ENL; NCOA3, FOXO3, and FOXO3; NCOA3, FOXO3, and PYGO1; NCOA3, FOXO3, and HSH2D; NCOA3, FOXO3, and NCOA2; NCOA3, FOXO3, and NOTCH2; NCOA3, PYGO1, and NCOA3; NCOA3, PYGO1, and ENL; NCOA3, PYGO1, and FOXO3; NCOA3, PYGO1, and PYGO1; NCOA3, PYGO1, and HSH2D; NCOA3, PYGO1, and NCOA2; NCOA3, PYGO1, and NOTCH2; NCOA3, HSH2D, and NCOA3; NCOA3, HSH2D, and ENL; NCOA3, HSH2D, and FOXO3; NCOA3, HSH2D, and PYGO1; NCOA3, HSH2D, and HSH2D; NCOA3, HSH2D, and NCOA2; NCOA3, HSH2D, and NOTCH2; NCOA3, NCOA2, and NCOA3; NCOA3, NCOA2, and ENL; NCOA3, NCOA2, and FOXO3; NCOA3, NCOA2, and PYGO1; NCOA3, NCOA2, and HSH2D; NCOA3, NCOA2, and NCOA2; NCOA3, NCOA2, and NOTCH2; NCOA3, NOTCH2, and NCOA3; NCOA3, NOTCH2, and ENL; NCOA3, NOTCH2, and FOXO3; NCOA3, NOTCH2, and PYGO1; NCOA3, NOTCH2, and HSH2D; NCOA3, NOTCH2, and NCOA2; NCOA3, NOTCH2, and NOTCH2; ENL, NCOA3, and NCOA3; ENL, NCOA3, and ENL; ENL, NCOA3, and FOXO3; ENL, NCOA3, and PYGO1; ENL, NCOA3, and HSH2D; ENL, NCOA3, and NCOA2; ENL, NCOA3, and NOTCH2; ENL, ENL, and NCOA3; ENL, ENL, and ENL; ENL, ENL, and FOXO3; ENL, ENL, and PYGO1; ENL, ENL, and HSH2D; ENL, ENL, and NCOA2; ENL, ENL, and NOTCH2; ENL, FOXO3, and NCOA3; ENL, FOXO3, and ENL; ENL, FOXO3, and FOXO3; ENL, FOXO3, and PYGO1; ENL, FOXO3, and HSH2D; ENL, FOXO3, and NCOA2; ENL, FOXO3, and NOTCH2; ENL, PYGO1, and NCOA3; ENL, PYGO1, and ENL; ENL, PYGO1, and FOXO3; ENL, PYGO1, and PYGO1; ENL, PYGO1, and HSH2D; ENL, PYGO1, and NCOA2; ENL, PYGO1, and NOTCH2; ENL, HSH2D, and NCOA3; ENL, HSH2D, and ENL; ENL, HSH2D, and FOXO3; ENL, HSH2D, and PYGO1; ENL, HSH2D, and HSH2D; ENL, HSH2D, and NCOA2; ENL, HSH2D, and NOTCH2; ENL, NCOA2, and NCOA3; ENL, NCOA2, and ENL; ENL, NCOA2, and FOXO3; ENL, NCOA2, and PYGO1; ENL, NCOA2, and HSH2D; ENL, NCOA2, and NCOA2; ENL, NCOA2, and NOTCH2; ENL, NOTCH2, and NCOA3; ENL, NOTCH2, and ENL; ENL, NOTCH2, and FOXO3; ENL, NOTCH2, and PYGO1; ENL, NOTCH2, and HSH2D; ENL, NOTCH2, and NCOA2; ENL, NOTCH2, and NOTCH2; FOXO3, NCOA3, and NCOA3; FOXO3, NCOA3, and ENL; FOXO3, NCOA3, and FOXO3; FOXO3, NCOA3, and PYGO1; FOXO3, NCOA3, and HSH2D; FOXO3, NCOA3, and NCOA2; FOXO3, NCOA3, and NOTCH2; FOXO3, ENL, and NCOA3; FOXO3, ENL, and ENL; FOXO3, ENL, and FOXO3; FOXO3, ENL, and PYGO1; FOXO3, ENL, and HSH2D; FOXO3, ENL, and NCOA2; FOXO3, ENL, and NOTCH2; FOXO3, FOXO3, and NCOA3; FOXO3, FOXO3, and ENL; FOXO3, FOXO3, and FOXO3; FOXO3, FOXO3, and PYGO1; FOXO3, FOXO3, and HSH2D; FOXO3, FOXO3, and NCOA2; FOXO3, FOXO3, and NOTCH2; FOXO3, PYGO1, and NCOA3; FOXO3, PYGO1, and ENL; FOXO3, PYGO1, and FOXO3; FOXO3, PYGO1, and PYGO1; FOXO3, PYGO1, and HSH2D; FOXO3, PYGO1, and NCOA2; FOXO3, PYGO1, and NOTCH2; FOXO3, HSH2D, and NCOA3; FOXO3, HSH2D, and ENL; FOXO3, HSH2D, and FOXO3; FOXO3, HSH2D, and PYGO1; FOXO3, HSH2D, and HSH2D; FOXO3, HSH2D, and NCOA2; FOXO3, HSH2D, and NOTCH2; FOXO3, NCOA2, and NCOA3; FOXO3, NCOA2, and ENL; FOXO3, NCOA2, and FOXO3; FOXO3, NCOA2, and PYGO1; FOXO3, NCOA2, and HSH2D; FOXO3, NCOA2, and NCOA2; FOXO3, NCOA2, and NOTCH2; FOXO3, NOTCH2, and NCOA3; FOXO3, NOTCH2, and ENL; FOXO3, NOTCH2, and FOXO3; FOXO3, NOTCH2, and PYGO1; FOXO3, NOTCH2, and HSH2D; FOXO3, NOTCH2, and NCOA2; FOXO3, NOTCH2, and NOTCH2; PYGO1, NCOA3, and NCOA3; PYGO1, NCOA3, and ENL; PYGO1, NCOA3, and FOXO3; PYGO1, NCOA3, and PYGO1; PYGO1, NCOA3, and HSH2D; PYGO1, NCOA3, and NCOA2; PYGO1, NCOA3, and NOTCH2; PYGO1, ENL, and NCOA3; PYGO1, ENL, and ENL; PYGO1, ENL, and FOXO3; PYGO1, ENL, and PYGO1; PYGO1, ENL, and HSH2D; PYGO1, ENL, and NCOA2; PYGO1, ENL, and NOTCH2; PYGO1, FOXO3, and NCOA3; PYGO1, FOXO3, and ENL; PYGO1, FOXO3, and FOXO3; PYGO1, FOXO3, and PYGO1; PYGO1, FOXO3, and HSH2D; PYGO1, FOXO3, and NCOA2; PYGO1, FOXO3, and NOTCH2; PYGO1, PYGO1, and NCOA3; PYGO1, PYGO1, and ENL; PYGO1, PYGO1, and FOXO3; PYGO1, PYGO1, and PYGO1; PYGO1, PYGO1, and HSH2D; PYGO1, PYGO1, and NCOA2; PYGO1, PYGO1, and NOTCH2; PYGO1, HSH2D, and NCOA3; PYGO1, HSH2D, and ENL; PYGO1, HSH2D, and FOXO3; PYGO1, HSH2D, and PYGO1; PYGO1, HSH2D, and HSH2D; PYGO1, HSH2D, and NCOA2; PYGO1, HSH2D, and NOTCH2; PYGO1, NCOA2, and NCOA3; PYGO1, NCOA2, and ENL; PYGO1, NCOA2, and FOXO3; PYGO1, NCOA2, and PYGO1; PYGO1, NCOA2, and HSH2D; PYGO1, NCOA2, and NCOA2; PYGO1, NCOA2, and NOTCH2; PYGO1, NOTCH2, and NCOA3; PYGO1, NOTCH2, and ENL; PYGO1, NOTCH2, and FOXO3; PYGO1, NOTCH2, and PYGO1; PYGO1, NOTCH2, and HSH2D; PYGO1, NOTCH2, and NCOA2; PYGO1, NOTCH2, and NOTCH2; HSH2D, NCOA3, and NCOA3; HSH2D, NCOA3, and ENL; HSH2D, NCOA3, and FOXO3; HSH2D, NCOA3, and PYGO1; HSH2D, NCOA3, and HSH2D; HSH2D, NCOA3, and NCOA2; HSH2D, NCOA3, and NOTCH2; HSH2D, ENL, and NCOA3; HSH2D, ENL, and ENL; HSH2D, ENL, and FOXO3; HSH2D, ENL, and PYGO1; HSH2D, ENL, and HSH2D; HSH2D, ENL, and NCOA2; HSH2D, ENL, and NOTCH2; HSH2D, FOXO3, and NCOA3; HSH2D, FOXO3, and ENL; HSH2D, FOXO3, and FOXO3; HSH2D, FOXO3, and PYGO1; HSH2D, FOXO3, and HSH2D; HSH2D, FOXO3, and NCOA2; HSH2D, FOXO3, and NOTCH2; HSH2D, PYGO1, and NCOA3; HSH2D, PYGO1, and ENL; HSH2D, PYGO1, and FOXO3; HSH2D, PYGO1, and PYGO1; HSH2D, PYGO1, and HSH2D; HSH2D, PYGO1, and NCOA2; HSH2D, PYGO1, and NOTCH2; HSH2D, HSH2D, and NCOA3; HSH2D, HSH2D, and ENL; HSH2D, HSH2D, and FOXO3; HSH2D, HSH2D, and PYGO1; HSH2D, HSH2D, and HSH2D; HSH2D, HSH2D, and NCOA2; HSH2D, HSH2D, and NOTCH2; HSH2D, NCOA2, and NCOA3; HSH2D, NCOA2, and ENL; HSH2D, NCOA2, and FOXO3; HSH2D, NCOA2, and PYGO1; HSH2D, NCOA2, and HSH2D; HSH2D, NCOA2, and NCOA2; HSH2D, NCOA2, and NOTCH2; HSH2D, NOTCH2, and NCOA3; HSH2D, NOTCH2, and ENL; HSH2D, NOTCH2, and FOXO3; HSH2D, NOTCH2, and PYGO1; HSH2D, NOTCH2, and HSH2D; HSH2D, NOTCH2, and NCOA2; HSH2D, NOTCH2, and NOTCH2; NCOA2, NCOA3, and NCOA3; NCOA2, NCOA3, and ENL; NCOA2, NCOA3, and FOXO3; NCOA2, NCOA3, and PYGO1; NCOA2, NCOA3, and HSH2D; NCOA2, NCOA3, and NCOA2; NCOA2, NCOA3, and NOTCH2; NCOA2, ENL, and NCOA3; NCOA2, ENL, and ENL; NCOA2, ENL, and FOXO3; NCOA2, ENL, and PYGO1; NCOA2, ENL, and HSH2D; NCOA2, ENL, and NCOA2; NCOA2, ENL, and NOTCH2; NCOA2, FOXO3, and NCOA3; NCOA2, FOXO3, and ENL; NCOA2, FOXO3, and FOXO3; NCOA2, FOXO3, and PYGO1; NCOA2, FOXO3, and HSH2D; NCOA2, FOXO3, and NCOA2; NCOA2, FOXO3, and NOTCH2; NCOA2, PYGO1, and NCOA3; NCOA2, PYGO1, and ENL; NCOA2, PYGO1, and FOXO3; NCOA2, PYGO1, and PYGO1; NCOA2, PYGO1, and HSH2D; NCOA2, PYGO1, and NCOA2; NCOA2, PYGO1, and NOTCH2; NCOA2, HSH2D, and NCOA3; NCOA2, HSH2D, and ENL; NCOA2, HSH2D, and FOXO3; NCOA2, HSH2D, and PYGO1; NCOA2, HSH2D, and HSH2D; NCOA2, HSH2D, and NCOA2; NCOA2, HSH2D, and NOTCH2; NCOA2, NCOA2, and NCOA3; NCOA2, NCOA2, and ENL; NCOA2, NCOA2, and FOXO3; NCOA2, NCOA2, and PYGO1; NCOA2, NCOA2, and HSH2D; NCOA2, NCOA2, and NCOA2; NCOA2, NCOA2, and NOTCH2; NCOA2, NOTCH2, and NCOA3; NCOA2, NOTCH2, and ENL; NCOA2, NOTCH2, and FOXO3; NCOA2, NOTCH2, and PYGO1; NCOA2, NOTCH2, and HSH2D; NCOA2, NOTCH2, and NCOA2; NCOA2, NOTCH2, and NOTCH2; NOTCH2, NCOA3, and NCOA3; NOTCH2, NCOA3, and ENL; NOTCH2, NCOA3, and FOXO3; NOTCH2, NCOA3, and PYGO1; NOTCH2, NCOA3, and HSH2D; NOTCH2, NCOA3, and NCOA2; NOTCH2, NCOA3, and NOTCH2; NOTCH2, ENL, and NCOA3; NOTCH2, ENL, and ENL; NOTCH2, ENL, and FOXO3; NOTCH2, ENL, and PYGO1; NOTCH2, ENL, and HSH2D; NOTCH2, ENL, and NCOA2; NOTCH2, ENL, and NOTCH2; NOTCH2, FOXO3, and NCOA3; NOTCH2, FOXO3, and ENL; NOTCH2, FOXO3, and FOXO3; NOTCH2, FOXO3, and PYGO1; NOTCH2, FOXO3, and HSH2D; NOTCH2, FOXO3, and NCOA2; NOTCH2, FOXO3, and NOTCH2; NOTCH2, PYGO1, and NCOA3; NOTCH2, PYGO1, and ENL; NOTCH2, PYGO1, and FOXO3; NOTCH2, PYGO1, and PYGO1; NOTCH2, PYGO1, and HSH2D; NOTCH2, PYGO1, and NCOA2; NOTCH2, PYGO1, and NOTCH2; NOTCH2, HSH2D, and NCOA3; NOTCH2, HSH2D, and ENL; NOTCH2, HSH2D, and FOXO3; NOTCH2, HSH2D, and PYGO1; NOTCH2, HSH2D, and HSH2D; NOTCH2, HSH2D, and NCOA2; NOTCH2, HSH2D, and NOTCH2; NOTCH2, NCOA2, and NCOA3; NOTCH2, NCOA2, and ENL; NOTCH2, NCOA2, and FOXO3; NOTCH2, NCOA2, and PYGO1; NOTCH2, NCOA2, and HSH2D; NOTCH2, NCOA2, and NCOA2; NOTCH2, NCOA2, and NOTCH2; NOTCH2, NOTCH2, and NCOA3; NOTCH2, NOTCH2, and ENL; NOTCH2, NOTCH2, and FOXO3; NOTCH2, NOTCH2, and PYGO1; NOTCH2, NOTCH2, and HSH2D; NOTCH2, NOTCH2, and NCOA2; or NOTCH2, NOTCH2, and NOTCH2, respectively.

In some embodiments, the first, second, and third transcriptional activation domains, respectively, are from PYGO1, FOXO3, and NCOA3, respectively. In some embodiments, the first, second, and third transcriptional activation domains, respectively, are from NOTCH2, FOXO3, and NCOA3, respectively. In some embodiments, the first, second, and third transcriptional activation domains, respectively, are from NCOA3, FOXO3, and NCOA3, respectively. In some embodiments, the first, second, and third transcriptional activation domains, respectively, are from HSH2D, FOXO3, and NCOA3, respectively. In some embodiments, the first, second, and third transcriptional activation domains, respectively, are from FOXO3, FOXO3, and NCOA3, respectively. In some embodiments, the first, second, and third transcriptional activation domains, respectively, are from NCOA2, FOXO3, and NCOA3, respectively. In some embodiments, the first, second, and third transcriptional activation domains, respectively, are from ENL, FOXO3, and NCOA3, respectively.

In some embodiments, any of the provided multipartite effector proteins, fusion proteins, and/or DNA targeting systems, such as a multipartite activator, comprises a combination of transcriptional activation domains, such as a combination of two or more of any of transcriptional activation domains shown in Table 1. In some embodiments, any of the provided multipartite effector proteins, fusion proteins, and/or DNA targeting systems, such as a multipartite activator, comprises a combination of two or more of any one of the SEQ ID NOS:set forth in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to any one of the SEQ ID NOS:set forth in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, any of the provided multipartite effector proteins, fusion proteins, and/or DNA targeting systems, such as a multipartite activator, comprises a combination of two or more of any one of the SEQ ID NOS:set forth in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, any of the provided multipartite effector proteins, fusion proteins, and/or DNA targeting systems, such as a multipartite activator, comprises two or more of any one of the SEQ ID NOS:set forth in Table 1.

In some embodiments, any of the provided multipartite effector proteins, fusion proteins, and/or DNA targeting systems, such as a multipartite activator, comprises a combination of transcriptional activation domains, such as a combination of three or more of any of transcriptional activation domains shown in Table 1. In some embodiments, any of the provided multipartite effector proteins, fusion proteins, and/or DNA targeting systems, such as a multipartite activator, comprises a combination of three or more of any one of the SEQ ID NOS:set forth in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof, or an amino acid sequence that has at least 90%, 91%, 92%, 93%, 94%, 95%, 96%, 97%, 98%, or 99% sequence identity to any one of the SEQ ID NOS:set forth in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, any of the provided multipartite effector proteins, fusion proteins, and/or DNA targeting systems, such as a multipartite activator, comprises a combination of three or more of any one of the SEQ ID NOS:set forth in Table 1, or a domain or a portion thereof, such as a contiguous portion thereof of at least 10, 15, 20, 22, 25, 30, 35, 37, 40, 42, 45, 47, 49, 50, 55, 57, 60, 61, 62, 65, 70, 72, 75, 76, or 80 amino acids, such as at least 20 amino acids, or a variant thereof. In some embodiments, any of the provided multipartite effector proteins, fusion proteins, and/or DNA targeting systems, such as a multipartite activator, comprises three or more of any one of the SEQ ID NOS:set forth in Table 1.

In some embodiments, the multipartite activator comprises the any one of the SEQ ID NOS:set forth in Table 2, or a domain, portion, or variant thereof, or an amino acid sequence that has at least 90%, 91%, 92%, 93%, 94%, 95%, 96%, 97%, 98%, or 99% sequence identity to any one of the SEQ ID NOS:set forth in Table 2. In some embodiments, the multipartite activator is or comprises any one of the SEQ ID NOS:set forth in Table 2. In some embodiments, the multipartite activator comprises a combination of transcriptional activation domains, such as any of the combinations of transcriptional activation domains shown in Table 2.

TABLE 2
Multipartite activators for Transcriptional Activation

| Bipartite/ | Transcriptional | Exemplary amino |
|---|---|---|
| Tripartite | Activation Domains | acid SEQ ID NO: |
| Bipartite | PYGO1, NCOA3 | 140 |
| Bipartite | NOTCH2, NCOA3 | 141 |
| Bipartite | NCOA3, NCOA3 | 142 |
| Bipartite | HSH2D, NCOA3 | 143 |
| Bipartite | FOXO3, NCOA3 | 144 |
| Bipartite | NCOA2, NCOA3 | 145 |
| Bipartite | ENL, NCOA3 | 146 |
| Bipartite | PYGO1, FOXO3 | 147 |
| Bipartite | NOTCH2, FOXO3 | 148 |
| Bipartite | NCOA3, FOXO3 | 149 |
| Bipartite | HSH2D, FOXO3 | 150 |
| Bipartite | FOXO3, FOXO3 | 151 |
| Bipartite | NCOA2, FOXO3 | 152 |
| Bipartite | ENL, FOXO3 | 153 |
| Tripartite | PYGO1, FOXO3, NCOA3 | 154 |
| Tripartite | NOTCH2, FOXO3, NCOA3 | 155 |
| Tripartite | NCOA3, FOXO3, NCOA3 | 156 |
| Tripartite | HSH2D, FOXO3, NCOA3 | 157 |
| Tripartite | FOXO3, FOXO3, NCOA3 | 158 |
| Tripartite | NCOA2, FOXO3, NCOA3 | 159 |
| Tripartite | ENL, FOXO3, NCOA3 | 160 |
| Tripartite | NCOA3, FOXO3, FOXO3 | 377 |

In some embodiments, the multipartite activator comprises any one of SEQ ID NOS: 140-160, or an amino acid sequence that has at least 90%, 91%, 92%, 93%, 94%, 95%, 96%, 97%, 98%, or 99% sequence identity to any one of SEQ ID NOS: 140-160. In some embodiments, the multipartite activator is set forth in any one of SEQ ID NOS: 140-160, or an amino acid sequence that has at least 90%, 91%, 92%, 93%, 94%, 95%, 96%, 97%, 98%, or 9900 sequence identity any one of SEQ ID NOS: 140-160, or a partially or fully functional fragment thereof, a domain thereof, or a portion thereof, such as a contiguous portion thereof of at least 10, 15, 20, 25, 30, 35, 40, 45, 50, 55, 60, 65, 70, 75, or 80 amino acids, or a variant thereof.

In some embodiments, the multipartite activator comprises domains from PYGO1 and NCOA3, respectively. In some embodiments, the multipartite activator comprises SEQ ID NO: 140, or an amino acid sequence that has at least 90%, 91%, 92%, 93%, 94%, 95%, 96%, 97%, 98%, or 99% sequence identity to SEQ ID NO: 140. In some embodiments, the multipartite activator is set forth in SEQ ID NO: 140.

In some embodiments, the multipartite activator comprises domains from NOTCH2 and NCOA3, respectively. In some embodiments, the multipartite activator comprises SEQ ID NO:141, or an amino acid sequence that has at least 90%, 91%, 92%, 93%, 94%, 95%, 96%, 97%, 98%, or 99% sequence identity to SEQ ID NO:141. In some embodiments, the multipartite activator is set forth in SEQ ID NO:141.

In some embodiments, the multipartite activator comprises domains from NCOA3 and NCOA3, respectively. In some embodiments, the multipartite activator comprises SEQ ID NO:142, or an amino acid sequence that has at least 90%, 91%, 92%, 93%, 94%, 95%, 96%, 97%, 98%, or 99% sequence identity to SEQ ID NO:142. In some embodiments, the multipartite activator is set forth in SEQ ID NO:142.

In some embodiments, the multipartite activator comprises domains from HSH2D and NCOA3, respectively. In some embodiments, the multipartite activator comprises SEQ ID NO:143, or an amino acid sequence that has at least 90%, 91%, 92%, 93%, 94%, 95%, 96%, 97%, 98%, or 99% sequence identity to SEQ ID NO:143. In some embodiments, the multipartite activator is set forth in SEQ ID NO:143.

In some embodiments, the multipartite activator comprises domains from FOXO3 and NCOA3, respectively. In some embodiments, the multipartite activator comprises SEQ ID NO:144, or an amino acid sequence that has at least 90%, 91%, 92%, 93%, 94%, 95%, 96%, 97%, 98%, or 99% sequence identity to SEQ ID NO:144. In some embodiments, the multipartite activator is set forth in SEQ ID NO:144.

In some embodiments, the multipartite activator comprises domains from NCOA2 and NCOA3, respectively. In some embodiments, the multipartite activator comprises SEQ ID NO:145, or an amino acid sequence that has at least 90%, 91%, 92%, 93%, 94%, 95%, 96%, 97%, 98%, or 99% sequence identity to SEQ ID NO:145. In some embodiments, the multipartite activator is set forth in SEQ ID NO:145.

In some embodiments, the multipartite activator comprises domains from ENL and NCOA3, respectively. In some embodiments, the multipartite activator comprises SEQ ID NO:146, or an amino acid sequence that has at least 90%, 91%, 92%, 93%, 94%, 95%, 96%, 97%, 98%, or 99% sequence identity to SEQ ID NO:146. In some embodiments, the multipartite activator is set forth in SEQ ID NO:146.

In some embodiments, the multipartite activator comprises domains from PYGO1 and FOXO3, respectively. In some embodiments, the multipartite activator comprises SEQ ID NO:147, or an amino acid sequence that has at least 90%, 91%, 92%, 93%, 94%, 95%, 96%, 97%, 98%, or 99% sequence identity to SEQ ID NO:147. In some embodiments, the multipartite activator is set forth in SEQ ID NO:147.

In some embodiments, the multipartite activator comprises domains from NOTCH2 and FOXO3, respectively. In some embodiments, the multipartite activator comprises SEQ ID NO:148, or an amino acid sequence that has at least 90%, 91%, 92%, 93%, 94%, 95%, 96%, 97%, 98%, or 99% sequence identity to SEQ ID NO:148. In some embodiments, the multipartite activator is set forth in SEQ ID NO:148.

In some embodiments, the multipartite activator comprises domains from NCOA3 and FOXO3, respectively. In some embodiments, the multipartite activator comprises SEQ ID NO:149, or an amino acid sequence that has at least 90%, 91%, 92%, 93%, 94%, 95%, 96%, 97%, 98%, or 99% sequence identity to SEQ ID NO:149. In some embodiments, the multipartite activator is set forth in SEQ ID NO:149.

In some embodiments, the multipartite activator comprises domains from HSH2D and FOXO3, respectively. In some embodiments, the multipartite activator comprises SEQ ID NO:150, or an amino acid sequence that has at least 90%, 91%, 92%, 93%, 94%, 95%, 96%, 97%, 98%, or 99% sequence identity to SEQ ID NO:150. In some embodiments, the multipartite activator is set forth in SEQ ID NO:150.

In some embodiments, the multipartite activator comprises domains from FOXO3 and FOXO3, respectively. In some embodiments, the multipartite activator comprises SEQ ID NO:151, or an amino acid sequence that has at least 90%, 91%, 92%, 93%, 94%, 95%, 96%, 97%, 98%, or 99% sequence identity to SEQ ID NO:151. In some embodiments, the multipartite activator is set forth in SEQ ID NO:151.

In some embodiments, the multipartite activator comprises domains from NCOA2 and FOXO3, respectively. In some embodiments, the multipartite activator comprises SEQ ID NO:152, or an amino acid sequence that has at least 90%, 91%, 92%, 93%, 94%, 95%, 96%, 97%, 98%, or 99% sequence identity to SEQ ID NO:152. In some embodiments, the multipartite activator is set forth in SEQ ID NO:152.

In some embodiments, the multipartite activator comprises domains from ENL and FOXO3, respectively. In some embodiments, the multipartite activator comprises SEQ ID NO:153, or an amino acid sequence that has at least 90%, 91%, 92%, 93%, 94%, 95%, 96%, 97%, 98%, or 99% sequence identity to SEQ ID NO:153. In some embodiments, the multipartite activator is set forth in SEQ ID NO:153.

In some embodiments, the multipartite activator comprises domains from PYGO1, FOXO3, and NCOA3, respectively. In some embodiments, the multipartite activator comprises SEQ ID NO:154, or an amino acid sequence that has at least 90%, 91%, 92%, 93%, 94%, 95%, 96%, 97%, 98%, or 99% sequence identity to SEQ ID NO:154. In some embodiments, the multipartite activator is set forth in SEQ ID NO:154.

In some embodiments, the multipartite activator comprises domains from NOTCH2, FOXO3, and NCOA3, respectively. In some embodiments, the multipartite activator comprises SEQ ID NO:155, or an amino acid sequence that has at least 90%, 91%, 92%, 93%, 94%, 95%, 96%, 97%, 98%, or 99% sequence identity to SEQ ID NO:155. In some embodiments, the multipartite activator is set forth in SEQ ID NO:155.

In some embodiments, the multipartite activator comprises domains from NCOA3, FOXO3, and NCOA3, respectively. In some embodiments, the multipartite activator comprises SEQ ID NO:156, or an amino acid sequence that has at least 90%, 91%, 92%, 93%, 94%, 95%, 96%, 97%, 98%, or 99% sequence identity to SEQ ID NO:156. In some embodiments, the multipartite activator is set forth in SEQ ID NO:156.

In some embodiments, the multipartite activator comprises domains from HSH2D, FOXO3, and NCOA3, respectively. In some embodiments, the multipartite activator comprises SEQ ID NO:157, or an amino acid sequence that has at least 90%, 91%, 92%, 93%, 94%, 95%, 96%, 97%, 98%, or 99% sequence identity to SEQ ID NO:157. In some embodiments, the multipartite activator is set forth in SEQ ID NO: 157.

In some embodiments, the multipartite activator comprises domains from FOXO3, FOXO3, and NCOA3, respectively. In some embodiments, the multipartite activator comprises SEQ ID NO:158, or an amino acid sequence that has at least 90%, 91%, 92%, 93%, 94%, 95%, 96%, 97%, 98%, or 99% sequence identity to SEQ ID NO:158. In some embodiments, the multipartite activator is set forth in SEQ ID NO:158.

In some embodiments, the multipartite activator comprises domains from NCOA2, FOXO3, and NCOA3, respectively. In some embodiments, the multipartite activator comprises SEQ ID NO:159, or an amino acid sequence that has at least 90%, 91%, 92%, 93%, 94%, 95%, 96%, 97%, 98%, or 99% sequence identity to SEQ ID NO:159. In some embodiments, the multipartite activator is set forth in SEQ ID NO:159.

In some embodiments, the multipartite activator comprises domains from ENL, FOXO3, and NCOA3, respectively. In some embodiments, the multipartite activator comprises SEQ ID NO:160, or an amino acid sequence that has at least 90%, 91%, 92%, 93%, 94%, 95%, 96%, 97%, 98%, or 99% sequence identity to SEQ ID NO:160. In some embodiments, the multipartite activator is set forth in SEQ ID NO:160.

In some embodiments, the multipartite activator comprises domains from NCOA3, FOXO3, and FOX03, respectively. In some embodiments, the multipartite activator comprises SEQ ID NO:377, or an amino acid sequence that has at least 90%, 91%, 92%, 93%, 94%, 95%, 96%, 97%, 98%, or 99% sequence identity to SEQ ID NO:377. In some embodiments, the multipartite activator is set forth in SEQ ID NO:377.

### II. Target Sites and Target Genes

In some embodiments, the transcriptional activation domains or multipartite effectors, such as multipartite activators disclosed herein, are targeted or recruited to a target site, such as a target site at an endogenous gene. In some embodiments, the transcriptional activation domains or multipartite effectors, such as multipartite activators, are targeted or recruited to the target site by any of the DNA-targeting systems and/or fusion proteins described herein. In some embodiments, the target site is at or comprised by an endogenous locus, such as a gene in a cell. In some embodiments, the target site is a target site in a target gene or regulatory DNA element or sequence thereof. In some embodiments, the target site is a target site for a target gene (i.e. the target site may be in the gene or a regulatory DNA element or sequence thereof). In some embodiments, the gene is in a cell, such as in the genome of a cell. In some embodiments, targeting or recruiting the transcriptional activation domain or multipartite activator to the target site leads to increased transcription of the target gene.

In some embodiments, the target site is in a cell, such as any suitable cell. In some embodiments, the cell is in or from any suitable organism, such as a human, mouse, dog, horse, rabbit, cattle, pig, hamster, gerbil, mouse, ferret, rat, cat, non-human primate, monkey, etc. In some embodiments, the cell is in or from a human. In some embodiments, the cell is any suitable cell, such as an immune cell (e.g. a T cell, B cell, or antigen-presenting cell), a liver cell (e.g. a hepatocyte), a cell of a nervous system (e.g. a neuron or glial cell), a heart cell (e.g. a cardiomyocyte) or a stem cell (e.g. an embryonic stem cell or induced pluripotent stem cell).

In some embodiments, the cell is modified or engineered. In some embodiments, the cell is in a subject (i.e. a cell in vivo). In some embodiments, the subject is a human. In some embodiments, the cell is from a subject or derived from a subject (e.g. the cell is a primary cell from a subject or is of a cell line derived from the subject). In some embodiments, the subject has or is suspected of having a condition, such as a disease or disorder. In some embodiments, increased transcription of the gene treats, reduces, or ameliorates the condition. In some embodiments, a therapy for treating the condition comprises increasing transcription of the gene.

In some embodiments, the target site is located in a regulatory DNA element of the target gene. In some embodiments, a regulatory DNA element is a sequence to which a gene regulatory protein may bind and affect transcription of the gene. In some embodiments, the regulatory DNA element is a cis, trans, distal, proximal, upstream, or downstream regulatory DNA element of a gene. In some embodiments, the regulatory DNA element is a promoter or enhancer of the gene. In some embodiments, the target site is located within a promoter, enhancer, exon, intron, untranslated region (UTR), 5′ UTR, or 3′ UTR of the gene. In some embodiments, a promoter is a nucleotide sequence to which RNA polymerase binds to begin transcription of the gene. In some embodiments, a promoter is a nucleotide sequence located within about 100 bp, about 500 bp, about 1000 bp, or more, of a transcriptional start site of the gene. In some embodiments the target site is located within a sequence of unknown or known function that is suspected of being able to control expression of a gene.

In some embodiments, the regulatory DNA element is a sequence to which any transcriptional activation domain, multipartite effector such as multipartite activator, fusion protein, or DNA-targeting system disclosed herein may target, bind, or be recruited to, thereby affecting (e.g. increasing or activating) transcription of a target gene, such as the gene in or near which the target site is located.

In some embodiments, the target gene is capable of regulating a phenotype in a cell. In some embodiments, transcriptional activation of the target gene can lead to or modulate one or more activities or functions of a cell, such as a phenotype. In some embodiments, the target gene is a gene for which increased expression of the gene regulates a cellular phenotype.

In some embodiments, the phenotype is associated with, related to, caused by, or therapeutic for a condition, such as a disease or disorder. In some aspects, the expression of the target gene and/or the dysregulation of expression of the target gene, such as the gene in or near which the target site is located, is associated with a disease or condition. In some aspects, a reduction or elimination of expression of the target gene is associated with a disease or condition. In some embodiments, increased expression of the target gene is therapeutic. In some embodiments, the phenotype is in a subject. For example, a subject may have a condition associated with or caused by decreased expression of the target gene. In some embodiments, the subject may have a condition that is treated, reduced, or ameliorated by increased expression of the target gene.

The target genes for increased transcription include any gene for which transcription and expression are increased in cells with a particular or desired function or activity, such as a cellular phenotype. Various methods may be utilized to characterize the transcription or expression levels of a gene in a cell, such as after the cell has been contacted or introduced with a provided transcriptional activation domain, multipartite effector such as multipartite activator, fusion protein, or DNA-targeting system, and optionally selected for a desired activity or function, such as cell phenotype. In some embodiments, analyzing the transcription activity or expression of a gene may be by RNA analysis. In some embodiments, the RNA analysis includes RNA quantification. In some embodiments, the RNA quantification occurs by reverse transcription quantitative PCR (RT-qPCR), multiplexed qRT-PCR, fluorescence in situ hybridization (FISH), high throughput RNA-sequencing (RNA-seq) or combinations thereof. In some embodiments, analyzing expression of a gene may be done by analyzing protein expression. In some embodiments, analysis of protein expression occurs by enzyme-linked immunoassay (ELISA), immunostaining, immunohistochemistry, flow cytometry, Bradford protein assay, or any other suitable method for analyzing protein expression.

In some embodiments, the gene is one in which expression of the gene in the cell is increased after having been contacted or introduced with a provided transcriptional activation domain, multipartite effector such as multipartite activator, fusion protein, or DNA-targeting system. In some embodiments, the increase in gene expression in the cell is about a log 2 fold change of greater than 1.0. For instance, the log 2 fold change is greater than at or about 1.5, at or about 2.0, at or about 2.5, at or about 3.0, at or about 4.0, at or about 5.0, at or about 6.0, at or about 7.0, at or about 8.0, at or about 9.0, at or about 10.0 or any value between any of the foregoing compared to the level of the gene in a control cell.

In some aspects, the phenotype is one that is characterized functionally. In some aspects, the phenotype can be characterized by one or more functions of the cells.

In some aspects, the phenotype is one that is characterized by a cell surface phenotype. It is understood that a cell that is positive (+) for a particular cell surface marker is a cell that expresses the marker on its surface at a level that is detectable. Likewise, it is understood that a cell that is negative (−) for a particular cell surface marker is a cell that expresses the marker on its surface at a level that is not detectable. Antibodies and other binding entities can be used to detect expression levels of marker proteins to identify or detect a given cell surface marker. Suitable antibodies may include polyclonal, monoclonal, fragments (such as Fab fragments), single chain antibodies and other forms of specific binding molecules. Antibody reagents for cell surface markers above are readily known to a skilled artisan. A number of well-known methods for assessing expression level of surface markers or proteins may be used, such as detection by affinity-based methods, e.g., immunoaffinity-based methods, e.g., in the context of surface markers, such as by flow cytometry. In some embodiments, the label is a fluorophore and the method for detection or identification of cell surface markers on cells (e.g. hepatocytes) is by flow cytometry. In some embodiments, different labels are used for each of the different markers by multicolor flow cytometry. In some embodiments, surface expression can be determined by flow cytometry, for example, by staining with an antibody that specifically binds to the marker and detecting the binding of the antibody to the marker.

In some embodiments, a cell is positive (pos or +) for a particular marker if there is detectable presence on or in the cell of a particular marker, which can be an intracellular marker or a surface marker. In some embodiments, surface expression is positive if staining by flow cytometry is detectable at a level substantially above the staining detected carrying out the same procedures with an isotype-matched control under otherwise identical conditions and/or at a level substantially similar to, or in some cases higher than, a cell known to be positive for the marker and/or at a level higher than that for a cell known to be negative for the marker. In some embodiments, a cell contacted by a DNA-targeting system described herein, has increased expression for a particular marker if the staining is substantially than a similar cell that was not contacted by the DNA-targeting system.

In some embodiments, a cell is negative (neg or −) for a particular marker if there is an absence of detectable presence on or in the cell of a particular marker, which can be an intracellular marker or a surface marker. In some embodiments, surface expression is negative if staining is not detectable by flow cytometry at a level substantially above the staining detected carrying out the same procedures with an isotype-matched control under otherwise identical conditions and/or at a level substantially lower than a cell known to be positive for the marker and/or at a level substantially similar to a cell known to be negative for the marker.

In some embodiments, the gene is a gene associated with a condition, such as a disease or disorder. In some embodiments, increased expression of the gene is therapeutic. Exemplary genes wherein increased expression could be therapeutic for a condition are shown in Table 3, along with the associated condition and tissue of gene expression.

TABLE 3
Exemplary genes for transcriptional activation

| Gene | Condition | Tissue |
|---|---|---|
| aromatic L-amino acid decarboxylase | Parkinson's disease | Brain |
| (AADC) | | |
| BCL2 Associated X (BAX) | Cancer | Tumor |
| brain-derived neurotrophic factor | Neurological conditions | Brain |
| (BDNF) | | |
| cystic fibrosis transmembrane | Cystic fibrosis | Lung |
| conductance regulator (CFTR) | | |
| factor IX | Hemophilia B | blood |
| factor VIII | Hemophilia A | blood |
| fragile X mental retardation 1 | Fragile X | Brain |
| (FMR1) | | |
| frataxin | Friedreich's ataxia | Brain |
| Growth factors (e.g., having a | Various | Various |
| protective or regenerative function) | | |
| HBG1/HBG2 | sickle cell anemia | Blood |
| IL-10 | Collitis, inflammatory bowel disease | Gut - T cells |
| IL1RA | rheumatoid arthritis | Cartilage |
| IL-2 | Various: graft versus host disease, | Various |
| | rheumatoid arthritis, lupus, type 1 diabetes | |
| mammary serine protease inhibitor | Cancer | Tumor |
| (maspin) | | |
| methyl-CpG-binding protein 2 | Rhett syndrome | Brain |
| (MECP2) | | |
| p53 | Cancer | Tumor |
| pigment epithelium-derived factor | Wet AMD, cancer | Eye, tumor |
| (PEDF) | | |
| platelet-derived growth factor | Tissue regeneration | Various - |
| (PDGF) | | muscle |
| sodium voltage-gated channel alpha | Dravet Syndrome | Brain |
| subunit 1 (SCN1A) | | |
| Triggering receptor expressed on | Alzheimer's disease | Brain |
| myeloid cells 2 (TREM-2) | | |
| ubiquitin-protein ligase E3A (Ube3a) | Angelman syndrome, Prader-Willi | Brain |
| | syndrome | |
| utrophin | Muscular dystrophy | Skeletal and |
| | | cardiac muscle |
| vascular endothelial growth factor | Tissue regeneration | Various - |
| (VEGF) | | muscle |

In some embodiments, the gene is a human frataxin (FXN) gene. Trinucleotide repeat expansion mutations in FXN can lead to decreased expression of FXN, causing Friedreich's ataxia. Friedreich's ataxia is a genetic, progressive, neurodegenerative movement disorder. In some embodiments, increased expression of FXN is therapeutic for a subject having or suspected of having Friedreich's ataxia. In some embodiments, the target site for a frataxin gene is located in a regulatory element of the frataxin gene, such as a promoter or enhancer.

In some embodiments, the target site is located within a FXN gene or regulatory DNA element thereof. In some embodiments, the target site is located within the genomic coordinates human genome assembly GRCh38 (hg38) chr9:68,940,179-69,205,519.

In some embodiments, the regulatory DNA element of FXN is a promoter. In some embodiments, the target site is located within 100 bp of a transcriptional start site of FXN. In some embodiments, the target site is located within the genomic coordinates hg38 chr9:69,034,622-69,036,670. In some embodiments, the target site is located within the genomic coordinates hg38 chr9:69,035,300-69,035,900. In some embodiments, the target site is located within the genomic coordinates chr9:69,034,900-69,035,900.

In some embodiments, the regulatory DNA element of FXN is an enhancer. In some embodiments, the target site is located within the genomic coordinates hg38 chr9:69,027,282-69,028,497. In some embodiments, the target site is located within the genomic coordinates hg38 chr9:69,027,615-69,028,101.

In some embodiments, the regulatory DNA element of FXN is an intronic enhancer, upstream enhancer, enhancer within a neighboring gene, downstream regulatory region, or other regulatory DNA element of FXN. In some embodiments, the target site is located within the genomic coordinates hg38 at chr9:69,044,201-69,045,347; chr9:69,030,752-69,031,507; chr9:68,999,262-69,000,023; chr9:69,085,468-69,086,426; chr9:69,096,701-69,097,567; chr9:69,120,690-69,123,549; or chr9:69,130,392-69,132,484.

In some embodiments, the target site for a frataxin gene is any target site listed in Table 4, or a subsequence thereof, as described in Section III.A.ii.

### III. DNA-Targeting Domains

In some embodiments, provided are DNA-targeting domains. In some aspects, a DNA-targeting domain is capable of specifically targeting (e.g. binding or hybridizing to) a target site, such as any target site described herein. In some aspects, a DNA-targeting domain targets a specific sequence of nucleotides, such as a DNA sequence. In some aspects, a DNA-targeting domain can be engineered (e.g. designed or programmed) to target a specific target site. In some aspects, a DNA-targeting domain recruits two or more transcriptional activation domains and/or a multipartite effector such as multipartite activator described herein to the target site, thereby inducing targeted gene activation.

In some embodiments, the DNA-targeting domain comprises a CRISPR associated (Cas) protein, zinc finger protein (ZFP), transcription activator-like effectors (TALE), meganuclease, homing endonuclease, I-SceI enzyme, or variants thereof. In some embodiments, the DNA-targeting domain comprises a catalytically inactive (e.g. nuclease-inactive or nuclease-inactivated) variant of any of the foregoing. In some embodiments, the DNA-targeting domain comprises a deactivated Cas9 (dCas9) protein or variant thereof that is a catalytically inactivated so that it is inactive for nuclease activity and is not able to cleave DNA.

In some embodiments, the DNA-targeting domain comprises a Cas-gRNA combination, comprising a Cas protein or variant thereof and at least one guide RNA (gRNA). In some embodiments, the gRNA binds to the target site. In some embodiments, the gRNA comprises a spacer sequence that is capable of targeting and/or hybridizing to the target site. In some embodiments, the gRNA is capable of complexing with the Cas protein or variant thereof, e.g. via a scaffold sequence of the gRNA. In some aspects, the gRNA directs or recruits the Cas protein or variant thereof to the target site.

Exemplary components and features of the DNA-targeting domains, including for CRISPR/Cas-based, ZFN-based, and TALE-based DNA-targeting domains are provided below.

### A. CRISPR/Cas-Based DNA-Targeting Domains

Provided herein are DNA-targeting domains based on CRISPR/Cas systems, i.e. CRISPR/Cas-based DNA-targeting domains, that are able to bind to a target site or a combination of target sites. In some embodiments, the CRISPR/Cas-based DNA-targeting domain is nuclease inactive, deactivated or nuclease-dead, such as a dCas (e.g. dCas9) so that the system binds to the target site without mediating nucleic acid cleavage. In some embodiments, the CRISPR/Cas-based DNA-targeting domain can include any known Cas protein or variant thereof, and generally a nuclease-inactive or dCas.

The CRISPR system (also known as CRISPR/Cas system, or CRISPR-Cas system) refers to a conserved microbial nuclease system, found in the genomes of bacteria and archaea, that provides a form of acquired immunity against invading phages and plasmids. Clustered Regularly Interspaced Short Palindromic Repeats (CRISPR), refers to loci containing multiple repeating DNA elements that are separated by non-repeating DNA sequences called spacers. Spacers are short sequences of foreign DNA that are incorporated into the genome between CRISPR repeats, serving as a ‘memory’ of past exposures. Spacers encode the DNA-targeting portion of RNA molecules that confer specificity for nucleic acid cleavage by the CRISPR system. CRISPR loci contain or are adjacent to one or more CRISPR-associated (Cas) genes, which can act as RNA-guided nucleases for mediating the cleavage, as well as non-protein coding DNA elements that encode RNA molecules capable of programming the specificity of the CRISPR-mediated nucleic acid cleavage.

In Type II CRISPR/Cas systems with the Cas protein Cas9, two RNA molecules and the Cas9 protein form a ribonucleoprotein (RNP) complex to direct Cas9 nuclease activity. The CRISPR RNA (crRNA) contains a spacer sequence that is complementary to a target nucleic acid sequence (target site), and that encodes the sequence specificity of the complex. The trans-activating crRNA (tracrRNA) base-pairs to a portion of the crRNA and forms a structure that complexes with the Cas9 protein, forming a Cas/RNA RNP complex.

Naturally occurring CRISPR/Cas systems, such as those with Cas9, have been engineered to allow efficient programming of Cas/RNA RNPs to target desired sequences in cells of interest, both for gene-editing and modulation of gene expression. The tracrRNA and crRNA have been engineered to form a single chimeric guide RNA molecule, commonly referred to as a guide RNA (gRNA), for example as described in WO 2013/176772, WO 2014/093661, WO 2014/093655, Jinek, M. et al. Science 337(6096):816-21 (2012), or Cong, L. et al. Science 339(6121):819-23 (2013), and as described herein, for example, in Section IIIA.ii. The spacer sequence of the gRNA can be chosen by a user to target the Cas/gRNA RNP complex to a desired locus, e.g. a desired target site in the target gene.

Cas proteins have also been engineered to be catalytically inactivated or nuclease inactive to allow targeting of Cas/gRNA RNPs without inducing cleavage at the target site. Mutations in Cas proteins can reduce or abolish nuclease activity of the Cas protein, rendering the Cas protein catalytically inactive. Cas proteins with reduced or abolished nuclease activity are referred to as deactivated Cas or dead Cas (dCas), or nuclease-inactive Cas (iCas) proteins, as referred to interchangeably herein. In some aspects, the dCas or iCas can still bind to target site in the DNA in a site- and/or sequence-specific manner, as long as it retains the ability to interact with the guide RNA (gRNA) which directs the Cas-gRNA combination to the target site.

dCas-fusion proteins with transcriptional and/or epigenetic regulators have been used as a versatile platform for ectopically regulating gene expression in target cells. These include fusion of a Cas with an effector domain, such as a transcriptional activator or transcriptional repressor. For example, fusing dCas9 with a transcriptional activator such as VP64 (a polypeptide composed of four tandem copies of VP16, a 16 amino acid transactivation domain of the Herpes simplex virus) can result in increased expression of a targeted gene. Alternatively, fusing dCas9 with a transcriptional repressor such as KRAB (Kruppel associated box) can result in reduced expression of a targeted gene. A variety of dCas-fusion proteins with transcriptional regulators have been engineered, for example as described in WO 2014/197748, WO 2016/130600, WO 2017/180915, WO 2021/226555, WO 2021/226077, WO 2013/176772, WO 2014/152432, WO 2014/093661, WO 2021/247570, Adli, M. Nat. Commun. 9, 1911 (2018), Perez-Pinera, P. et al. Nat. Methods 10, 973-976 (2013), Mali, P. et al. Nat. Biotechnol. 31, 833-838 (2013), Maeder, M. L. et al. Nat. Methods 10, 977-979 (2013), Gilbert, L. A. et al. Cell 154(2):442-451 (2013), and Nuñez, J. K. et al. Cell 184(9):2503-2519 (2021).

i. Cas Proteins

In some aspects, the DNA-targeting domain comprises a CRISPR-associated (Cas) protein. In some embodiments, the Cas protein is a variant Cas protein, such as a Cas protein derived from or based on a naturally occurring Cas protein or portion thereof. In some embodiments, the variant Cas protein comprises one or more modifications, mutations, or amino acid substitutions in comparison to the naturally occurring Cas protein. In particular embodiments provided herein, the Cas protein is nuclease-inactive (i.e. is a dCas protein).

In some embodiments, the Cas protein is derived from a Class 1 CRISPR system (i.e. multiple Cas protein system), such as a Type I, Type III, or Type IV CRISPR system. In some embodiments, the Cas protein is derived from a Class 2 CRISPR system (i.e. single Cas protein system), such as a Type II, Type V, or Type VI CRISPR system. In some embodiments, the Cas protein is derived from a Type V CRISPR system.

CRISPR/Cas systems may be multi-protein systems or single effector protein systems. Multi-protein, or Class 1, CRISPR systems include Type I, Type III, and Type IV systems. In some aspects, Class 2 systems include a single effector molecule and include Type II, Type V, and Type VI. In some embodiments, the DNA targeting system comprises components of CRISPR/Cas systems, such as a Type I, Type II, Type III, Type IV, Type V, or Type VI CRISPR system. In some embodiments, the Cas protein is from a Class 1 CRISPR system (i.e. multiple Cas protein system), such as a Type I, Type III, or Type IV CRISPR system. In some embodiments, the Cas protein is from a Class 2 CRISPR system (i.e. single Cas protein system), such as a Type II, Type V, or Type VI CRISPR system.

Various CRISPR/Cas systems and associated Cas proteins for use in gene editing and regulation have been described, for example in Moon et al. Exp. Mol. Med. 51, 1-11 (2019), Zhang, F. Q. Rev. Biophys. 52, E6 (2019), and Makarova et al. Methods Mol. Biol. 1311:47-75 (2015).

Type I CRISPR/Cas systems employ a large multisubunit ribonucleoprotein (RNP) complex called Cascade that recognizes double-stranded DNA (dsDNA) targets. After target recognition and verification, Cascade recruits the signature protein Cas3, a fused helicase-nuclease, to degrade DNA.

In some embodiments, the Cas protein is from a Type II CRISPR system. Exemplary Cas proteins of a Type II CRISPR system include Cas9. In some embodiments, the Cas protein is from a Cas9 protein or variant thereof, for example as described in WO 2013/176772, WO 2014/152432, WO 2014/093661, WO 2014/093655, Jinek. et al. Science 337(6096):816-21 (2012), Mali et al. Science 339(6121):823-6 (2013), Cong et al. Science 339(6121):819-23 (2013), Perez-Pinera et al. Nat. Methods 10, 973-976 (2013), or Mali et al. Nat. Biotechnol. 31, 833-838 (2013). In Type II CRISPR/Cas systems with the Cas protein Cas9, two RNA molecules and the Cas9 protein form a ribonucleoprotein (RNP) complex to direct Cas9 nuclease activity. The CRISPR RNA (crRNA) contains a spacer sequence that is complementary to a target nucleic acid sequence (target site), and that encodes the sequence specificity of the complex. The trans-activating crRNA (tracrRNA) base-pairs to a portion of the crRNA and forms a structure that complexes with the Cas9 protein, forming a Cas/RNA RNP complex. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage.

Different Type II systems have differing PAM requirements. The S. pyogenes CRISPR system may have the PAM sequence for this Cas9 (SpCas9) as 5′-NRG-3′, where R is either A or G, and characterized the specificity of this system in human cells. A unique capability of the CRISPR/Cas9 system is the straightforward ability to simultaneously target multiple distinct genomic loci by co-expressing a single Cas9 protein with two or more sgRNAs. For example, the Streptococcus pyogenes Type II system typically prefers to use an “NGG” sequence, where “N” can be any nucleotide, but also accepts other PAM sequences, such as “NAG” in engineered systems (Hsu et al., Nature Biotechnology (2013) doi:10.1038/nbt.2647). Similarly, the Cas9 derived from Neisseria meningitidis (NmCas9) normally has a native PAM of NNNNGATT (SEQ ID NO:52), but has activity across a variety of PAMs, including a highly degenerate NNNNGNNN (SEQ ID NO:302) PAM (Esvelt et al. Nature Methods (2013) doi:10.1038/nmeth.2681). In another example, the Cas9 derived from Campylobacter jejuni typically uses 5′-NNNNACAC-3′ (SEQ ID NO:306) or 5′-NNNNRYAC-3′ (SEQ ID NO:53) PAM sequences, where “N” can be any nucleotide, “R” can be either guanine (G) or adenine (A), and “Y” can be either cytosine (C) or thymine (T). In some aspects, the PAM sequences for spacer targeting depends on the type, ortholog, variant or species of the Cas protein.

In some embodiments, the Cas protein is derived from a Cas9 protein or variant thereof, for example as described in WO 2013/176772, WO 2014/152432, WO 2014/093661, WO 2014/093655, Jinek, M. et al. Science 337(6096):816-21 (2012), Mali, P. et al. Science 339(6121):823-6 (2013), Cong, L. et al. Science 339(6121):819-23 (2013), Perez-Pinera, P. et al. Nat. Methods 10, 973-976 (2013), or Mali, P. et al. Nat. Biotechnol. 31, 833-838 (2013). Various CRISPR/Cas systems and associated Cas proteins for use in gene editing and regulation have been described, for example in Moon, S. B. et al. Exp. Mol. Med. 51, 1-11 (2019), Zhang, F. Q. Rev. Biophys. 52, E6 (2019), and Makarova K. S. et al. Methods Mol. Biol. 1311:47-75 (2015).

In some embodiments, the Cas9 protein comprises a sequence from a Cas9 molecule of S. aureus. In some embodiments, the Cas9 protein comprises a sequence set forth in SEQ ID NO:3 or SEQ ID NO:4, or a variant thereof, such as an amino acid sequence that has at least 90%, 91%, 92%, 93%, 94%, 95%, 96%, 97%, 98%, or 99% sequence identity to SEQ ID NO:3 or SEQ ID NO:4. In some embodiments, the Cas9 protein comprises a sequence from a Cas9 molecule of S. pyogenes. In some embodiments, the Cas9 protein comprises a sequence set forth in SEQ ID NO:7 or SEQ ID NO:8, or a variant thereof, such as an amino acid sequence that has at least 90%, 91%, 92%, 93%, 94%, 95%, 96%, 97%, 98%, or 99% sequence identity to SEQ ID NO:7 or SEQ ID NO:8.

In Type III systems, the RNP complex is multimeric with a helicoid structure similar to Cascade. In contrast to Type I CRISPR/Cas systems, the Type III RNP complex recognizes complementary RNA sequences instead of dsDNA. RNA recognition stimulates a nonspecific DNA cleavage activity of the exemplary Type III Cas10 nuclease that is part of the RNP complex, such that DNA cleavage is achieved cotranscriptionally.

In some embodiments, the Cas protein is from a Type V CRISPR system. Exemplary Cas proteins of a Type V CRISPR system include Cas12a (also known as Cpf1), Cas12b (also known as C2cl), Cas12e (also known as CasX), Cas12k (also known as C2c5), Cas14a, and Cas14b. In some embodiments, the Cas protein is from a Cas12 protein (i.e. Cpf1) or variant thereof, for example as described in WO 2017/189308, WO2019/232069 and Zetsche et al. Cell. 163(3):759-71 (2015).

Exemplary Type V systems include those based on a Cas12 effector, and the C-terminus with only one RuvC endonuclease domain is the defining characteristic of the Type V systems. The RuvC nuclease domain cleaves dsDNA adjacent to protospacer adjacent motif (PAM) sequences and single-stranded DNA (ssDNA) nonspecifically. The Type V systems can be further divided into subtypes, each characterized by different signature proteins, PAM sequences, and properties. Non-limiting exemplary Cas proteins derived from Type V CRISPR systems include Cas12a (Cpf1), Un1Cas12f1, Cas12j (CasPhi, such as CasPhi-2), Cas12k, and CasMini. For example, Type V-A includes, for example, Cas12a, which uses “TTTV” (SEQ ID NO:56) PAM sequence, where “V” is adenine (A), cytosine (C), or guanine (G). Type V-F is includes, for example, Cas12f, which can use “TTTR” (SEQ ID NO:308), where “R” is G or A, or “TTTN” (SEQ ID NO:305), where “N” is any nucleotide. Type V-K is includes, for example, Cas12k, which uses “GGTT” (SEQ ID NO:307) PAM sequence.

In some embodiments, the Cas12a protein comprises a sequence from a Cas12a molecule of Acidaminococcus sp, such as an AsCas12a set forth in SEQ ID NO:327 or SEQ ID NO:328, or a variant thereof, such as an amino acid sequence that has at least 90%, 91%, 92%, 93%, 94%, 95%, 96%, 97%, 98%, or 99% sequence identity to SEQ ID NO:327 or SEQ ID NO:328.

Non-limiting examples of Cas9 orthologs from other bacterial strains include but are not limited to: Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS10; Burkholderiales bacterium 1_1_47; Caldicelulosiruptor becscii DSM 6725; Campylobacter jejuni; Candidatus Desulforudis audaxviator MP104C; Caldicellulosiruptor hydrothermalis 108; Clostridium phage c-st; Clostridium botulinum A3 str. Loch Maree; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMM01; Microcystis aeruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum_thermopropionicum SI; Petrotoga mobilis SJ95; Polaromonas naphthalenivorans CJ2; Polaromonas sp. JS666; Pseudoalteromonas haloplanktis TAC125; Streptomyces pristinaespiralis ATCC 25486; Streptomyces pristinaespiralis ATCC 25486; Streptococcus thermophilus; Streptomyces viridochromogenes DSM 40736; Streptosporangium roseum DSM 43021; Synechococcus sp. PCC 7335; and Thermosipho africanus TCF52B (Chylinski et al., RNA Biol., 2013; 10(5): 726-737).

In some embodiments, the DNA-targeting systems or fusion proteins comprise a Cas protein, such as a Cas protein set forth in any one of SEQ ID NOS:3, 4, 7, 8, 329, 330, 333-336, and 341-344, or a variant thereof, such as an amino acid sequence that has at least 90%, 91%, 92%, 93%, 94%, 95%, 96%, 97%, 98%, or 99% sequence identity to any one of SEQ ID NOS:3, 4, 7, 8, 329, 330, 333-336, and 341-344. In some embodiments, the Cas protein of any of the DNA-targeting systems or fusion proteins provided herein comprise a sequence set forth in any one of SEQ ID NOS:3, 4, 7, 8, 329, 330, 333-336, and 341-344, or a variant thereof, such as an amino acid sequence that has at least 90%, 91%, 92%, 93%, 94%, 95%, 96%, 97%, 98%, or 99% sequence identity to any one of SEQ ID NOS:3, 4, 7, 8, 329, 330, 333-336, and 341-344. In some aspects, the Cas protein lacks an initial methionine residue. In some aspects, the Cas protein comprises an initial methionine residue.

In some aspects, the Cas protein is a variant that lacks nuclease activity (i.e. is a dCas protein). In some embodiments, the Cas protein is mutated so that nuclease activity is reduced or eliminated. Such Cas proteins are referred to as deactivated Cas or dead Cas (dCas) or nuclease-inactive Cas (iCas) proteins, as referred to interchangeably herein. In some embodiments, the Cas protein is a variant Cas9 protein that lacks nuclease activity or that is a deactivated Cas9 (dCas9, or iCas9) protein.

In some aspects, in the provided DNA-targeting systems and fusion proteins, the DNA-targeting domain, e.g., Cas, is a deactivated Cas (dCas), or a nuclease-inactive Cas (iCas). In some embodiments, the component of the DNA-targeting domain, such as a protein component, comprises a Cas9 variant such as a deactivated Cas9 or inactivated Cas9. In some embodiments, the component of the DNA-targeting domain, such as a protein component, comprises a Cas12a variant such as a deactivated Cas12a (Cpf1) or inactivated Cas12a (Cpf1). In some aspects, the Cas9 protein may be mutated so that the nuclease activity is deactivated or inactivated (also referred to as dCas9 or iCas9). In some aspects, the Cas protein is a variant that lacks nuclease activity (i.e. is a dCas protein). In some embodiments, the Cas protein is mutated so that nuclease activity is reduced or eliminated. Such Cas proteins are referred to as deactivated Cas or dead Cas (dCas) or nuclease-inactive Cas (iCas) proteins, as referred to interchangeably herein. In some embodiments, the variant Cas protein is a variant Cas9 protein that lacks nuclease activity or that is a deactivated Cas9 (dCas9, or iCas9) protein. In some embodiments, the variant Cas protein is a variant Cpf1 protein that lacks nuclease activity or that is a deactivated Cas12a (dCas12a, or iCas12a) protein.

In some embodiments, Cas proteins are engineered to be catalytically inactivated or nuclease inactive to allow targeting of Cas/gRNA RNPs without inducing cleavage at the target site. Mutations in Cas proteins can reduce or abolish nuclease activity of the Cas protein, rendering the Cas protein catalytically inactive. Cas proteins with reduced or abolished nuclease activity are referred to as deactivated Cas (dCas), or nuclease-inactive Cas (iCas) proteins, as referred to interchangeably herein. In some aspects, the dCas or iCas can still bind to target site in the DNA in a site- and/or sequence-specific manner, as long as it retains the ability to interact with the guide RNA (gRNA) which directs the Cas-gRNA combination to the target site.

In some aspects, the dCas or iCas exhibits reduced or no endodeoxyribonuclease activity. For example, an exemplary dCas or iCas, for example dCas9 or iCas9, exhibits less than about 20%, less than about 15%, less than about 10%, less than about 5%, less than about 1%, or less than about 0.1%, of the endodeoxyribonuclease activity of a wild-type Cas protein, e.g., a wild-type Cas9 protein. In some embodiments, the dCas or iCas, for example dCas9 or iCas9, exhibits substantially no detectable endodeoxyribonuclease activity. In some embodiments, an exemplary dCas or iCas, for example dCas9 or iCas9, comprises one or more amino acid mutations, substitutions, deletions or insertions at a position corresponding to a position selected from D10, G12, G17, E762, H840, N854, N863, H982, H983, A984, D986, and/or a A987, with reference to a wild-type Streptococcus pyogenes Cas9 (SpCas9), for example, with reference to numbering of positions of a SpCas9 sequence set forth in SEQ ID NO:7. In some aspects, the dCas9 or iCas9 comprises one or more amino acid mutations, substitutions, deletions or insertions corresponding to D10A, G12A, G17A, E762A, H840A, N854A, N863A, H982A, H983A, A984A, and/or D986A, with reference to a wild-type Streptococcus pyogenes Cas9 (SpCas9), for example, with reference to numbering of positions of a SpCas9 sequence set forth in SEQ ID NO:7. Corresponding positions for mutations can be determined based on sequence alignments and determination of sequence conservation, for example, as described in WO 2013/171772 for Cas9 proteins from various species. In some aspects, the dCas protein lacks an initial methionine residue. In some aspects, the dCas protein comprises an initial methionine residue.

In some embodiments, the dCas9 protein comprises a sequence derived from a naturally occurring Cas9 molecule, or variant thereof. In some embodiments, the dCas9 protein can comprise a sequence derived from a naturally occurring Cas9 molecule of S. pyogenes, S. thermophilus, S. aureus, C. jejuni, N. meningitidis, F. novicida, S. canis, S. auricularis, or variant thereof. In some embodiments, the dCas9 protein comprises a sequence derived from a naturally occurring Cas9 molecule of S. aureus. In some embodiments, the dCas9 protein comprises a sequence derived from a naturally occurring Cas9 molecule of S. pyogenes. In some embodiments, the dCas9 protein comprises a sequence from a Cas9 molecule of C. jejuni.

Exemplary deactivated Cas9 (dCas9) derived from S. pyogenes contains silencing mutations of the RuvC and HNH nuclease domains (D10A and H840A), for example as described in WO 2013/176772, WO 2014/093661, Jinek et al. Science 337(6096):816-21 (2012), and Qi et al. Cell 152(5):1173-83 (2013). Exemplary dCas variants derived from the Cas12 system (i.e. Cpf1) are described, for example in WO 2017/189308 and Zetsche et al. Cell 163(3):759-71 (2015). Conserved domains that mediate nucleic acid cleavage, such as RuvC and HNH endonuclease domains, are readily identifiable in Cas orthologues, and can be mutated to produce inactive variants, for example as described in Zetsche et al. Cell 163(3):759-71 (2015). Other exemplary Cas orthologs or variants include engineered variants based on a Cas12f (also known as Cas14), including those described in Xu et al., Mol. Cell 81(20):4333-4345 (2021).

In some embodiments, the DNA-targeting domain comprises a Cas-gRNA combination that includes (a) a Cas protein or a variant thereof and (b) at least one gRNA. In some embodiments, the variant Cas protein lacks nuclease activity or is a deactivated Cas (dCas) protein. In some embodiments, the gRNA is capable of complexing with the Cas protein or variant thereof. In some embodiments, the gRNA comprises a gRNA spacer sequence that is capable of hybridizing to the target site or is complementary to the target site at a target gene.

In some embodiments, the Cas protein or a variant thereof is a Cas9 protein or a variant thereof. In some embodiments, the variant Cas protein is a variant Cas9 protein that lacks nuclease activity or that is a deactivated Cas9 (dCas9) protein. In some embodiments, the Cas9 protein or a variant thereof is a Staphylococcus aureus Cas9 (SaCas9) protein or a variant thereof. In some embodiments, the variant Cas9 protein is a Staphylococcus aureus dCas9 protein (dSaCas9) that comprises at least one amino acid mutation selected from D10A and N580A, with reference to numbering of positions of SEQ ID NO:3. In some embodiments, the variant Cas9 protein comprises the sequence set forth in SEQ ID NO:2, or an amino acid sequence that has at least 90%, 91%, 92%, 93%, 94%, 95%, 96%, 97%, 98%, or 99% sequence identity thereto. In some embodiments, the variant Cas9 protein comprises the sequence set forth in SEQ ID NO:2, which lacks an initial methionine residue. In some embodiments, the variant Cas9 protein comprises the sequence set forth in SEQ ID NO:309, which includes an initial methionine residue.

In some embodiments, the Cas9 protein or variant thereof is a Streptococcus pyogenes Cas9 (SpCas9) protein or a variant thereof. In some embodiments, the variant Cas9 is a Streptococcus pyogenes dCas9 (dSpCas9) protein that comprises at least one amino acid mutation selected from D10A and H840A, with reference to numbering of positions of SEQ ID NO:7. In some embodiments, the variant Cas9 protein comprises the sequence set forth in SEQ ID NO:6, or an amino acid sequence that has at least 90%, 91%, 92%, 93%, 94%, 95%, 96%, 97%, 98%, or 99% sequence identity thereto. In some embodiments, the variant Cas9 protein comprises the sequence set forth in SEQ ID NO:6, which lacks an initial methionine residue. In some embodiments, the variant Cas9 protein comprises the sequence set forth in SEQ ID NO:310, which includes an initial methionine residue.

In some embodiments, the Cas9 protein or variant thereof is a Campylobacter jejuni Cas9 (CjCas9) protein or a variant thereof. In some embodiments, the variant Cas9 comprises at least one amino acid mutation compared to the sequence set forth in SEQ ID NO:341 or 342. In some embodiments, the variant Cas9 protein comprises the sequence set forth in SEQ ID NO:339, or an amino acid sequence that has at least 90%, 91%, 92%, 93%, 94%, 95%, 96%, 97%, 98%, or 99% sequence identity thereto. In some embodiments, the variant Cas9 protein comprises the sequence set forth in SEQ ID NO:340, or an amino acid sequence that has at least 90%, 91%, 92%, 93%, 94%, 95%, 96%, 97%, 98%, or 99% sequence identity thereto. In some embodiments, the variant Cas9 protein comprises the sequence set forth in SEQ ID NO:342, which lacks an initial methionine residue. In some embodiments, the variant Cas9 protein comprises the sequence set forth in SEQ ID NO:341, which includes an initial methionine residue.

In some embodiments, the Cas protein or a variant thereof is a Cas12a protein or a variant thereof. In some embodiments, the variant Cas protein is a variant Cas12a protein that lacks nuclease activity or that is a deactivated Cas12a (dCas12a) protein. In some embodiments, the Cas12a protein or variant thereof is a Acidaminococcus sp. Cas12a (AsCas12a) protein or a variant thereof. In some embodiments, the variant Cas12a is a Acidaminococcus sp. dCas12a (dAsCas12a) protein that comprises at least one amino acid mutation compared to the sequence set forth in SEQ ID NO:329 or 330. In some embodiments, the variant Cas12a protein comprises the sequence set forth in SEQ ID NO:327, or an amino acid sequence that has at least 90%, 91%, 92%, 93%, 94%, 95%, 96%, 97%, 98%, or 99% sequence identity thereto. In some embodiments, the variant Cas12a protein comprises the sequence set forth in SEQ ID NO:328, or an amino acid sequence that has at least 90%, 91%, 92%, 93%, 94%, 95%, 96%, 97%, 98%, or 99% sequence identity thereto. In some embodiments, the variant Cas12a protein comprises the sequence set forth in SEQ ID NO:328, which lacks an initial methionine residue. In some embodiments, the variant Cas12a protein comprises the sequence set forth in SEQ ID NO:327, which includes an initial methionine residue.

In some embodiments, the Cas protein or a variant thereof is a CasPhi-2 protein or a variant thereof. In some embodiments, the variant Cas protein is a variant CasPhi-2 protein that lacks nuclease activity or that is a deactivated CasPhi-2 (dCasPhi-2) protein. In some embodiments, the variant CasPhi-2 comprises at least one amino acid mutation compared to the sequence set forth in SEQ ID NO:333 or 334. In some embodiments, the variant CasPhi-2 protein comprises the sequence set forth in SEQ ID NO:331, or an amino acid sequence that has at least 90%, 91%, 92%, 93%, 94%, 95%, 96%, 97%, 98%, or 99% sequence identity thereto. In some embodiments, the variant CasPhi-2 protein comprises the sequence set forth in SEQ ID NO:332, or an amino acid sequence that has at least 90%, 91%, 92%, 93%, 94%, 95%, 96%, 97%, 98%, or 99% sequence identity thereto. In some embodiments, the variant CasPhi-2 protein comprises the sequence set forth in SEQ ID NO:332, which lacks an initial methionine residue. In some embodiments, the variant CasPhi-2 protein comprises the sequence set forth in SEQ ID NO:331, which includes an initial methionine residue.

In some embodiments, the Cas protein or a variant thereof is a Un1Cas12f1 protein or a variant thereof. In some embodiments, the variant Cas protein is a variant Un1Cas12f1 protein that lacks nuclease activity or that is a deactivated Un1Cas12f1 (dUn1Cas12f1) protein. In some embodiments, the variant Un1Cas12f1 comprises at least one amino acid mutation compared to the sequence set forth in SEQ ID NO:335 or 336. In some embodiments, the variant Un1Cas12f1 protein comprises the sequence set forth in SEQ ID NO:337, or an amino acid sequence that has at least 90%, 91%, 92%, 93%, 94%, 95%, 96%, 97%, 98%, or 99% sequence identity thereto. In some embodiments, the variant Un1Cas12f1 protein comprises the sequence set forth in SEQ ID NO:338, or an amino acid sequence that has at least 90%, 91%, 92%, 93%, 94%, 95%, 96%, 97%, 98%, or 99% sequence identity thereto. In some embodiments, the variant Un1Cas12f1 protein comprises the sequence set forth in SEQ ID NO:338, which lacks an initial methionine residue. In some embodiments, the variant Un1Cas12f1 protein comprises the sequence set forth in SEQ ID NO:337, which includes an initial methionine residue.

In some embodiments, the Cas protein or a variant thereof is a Cas12k protein or a variant thereof. In some embodiments, the Cas12k protein comprises the sequence set forth in SEQ ID NO:343, or an amino acid sequence that has at least 90%, 91%, 92%, 93%, 94%, 95%, 96%, 97%, 98%, or 99% sequence identity thereto. In some embodiments, the Cas12k protein comprises the sequence set forth in SEQ ID NO:344, or an amino acid sequence that has at least 90%, 91%, 92%, 93%, 94%, 95%, 96%, 97%, 98%, or 99% sequence identity thereto. In some embodiments, the Cas12k protein comprises the sequence set forth in SEQ ID NO:344, which lacks an initial methionine residue. In some embodiments, the Cas12k protein comprises the sequence set forth in SEQ ID NO:343, which includes an initial methionine residue.

In some embodiments, the Cas protein or a variant thereof is a CasMini protein or a variant thereof, such as an engineered Cas protein or variant based on a Cas12f (also known as Cas14), including those described in Xu et al., Mol. Cell 81(20):4333-4345 (2021) or set forth in SEQ ID NO:303. In some embodiments, the variant Cas protein is a variant CasMini protein that lacks nuclease activity or that is a deactivated CasMini (dCasMini) protein. In some embodiments, the variant CasMini comprises at least one amino acid mutation compared to the sequence set forth in SEQ ID NO:303. In some embodiments, the variant CasMini protein comprises the sequence set forth in SEQ ID NO:303, or an amino acid sequence that has at least 90%, 91%, 92%, 93%, 94%, 95%, 96%, 97%, 98%, or 99% sequence identity thereto. In some embodiments, the CasMini protein comprises the sequence set forth in SEQ ID NO:303. In some embodiments, the variant CasMini protein comprises the sequence set forth in SEQ ID NO:345 or 346, or an amino acid sequence that has at least 90%, 91%, 92%, 93%, 94%, 95%, 96%, 97%, 98%, or 99% sequence identity thereto. In some embodiments, the CasMini protein comprises the sequence set forth in SEQ ID NO:345, which lacks an initial methionine residue. In some embodiments, the CasMini protein comprises the sequence set forth in SEQ ID NO:346, which includes an initial methionine residue.

DNA-targeting systems, in some cases comprising a fusion protein, such as dCas-fusion proteins include fusion of the Cas with an effector domain, such as a transcription activation domain. Any of a variety of effector domains, for example those that increase transcription from the target locus, including any described herein, for example, in Section II.D, can be used.

In some aspects, provided is a DNA-targeting system comprising a fusion protein comprising a DNA-targeting domain comprising a nuclease-inactive Cas protein or variant thereof, and an effector domain for increasing or inducing transcriptional activation (i.e. a transcriptional activator) when targeted to a target site in a FXN gene or regulatory element thereof. In some aspects, the DNA-targeting system also includes one or more gRNA, provided in combination or as a complex with the dCas protein or variant thereof, for targeting of the DNA-targeting system to the target site. In some embodiments, the fusion protein is guided to a specific target site sequence of the target gene by the guide RNA, wherein the effector domain mediates targeted epigenetic modification to increase or promote transcription of the target gene.

ii. Guide RNAs

In some embodiments, the Cas protein (e.g. dCas9) is provided in combination or as a complex with one or more guide RNA (gRNA). In some aspects, the gRNA is a nucleic acid that promotes the specific targeting or homing of the gRNA/Cas ribonucleoprotein (RNP) complex to the target site of the target gene, such as any described above. In some embodiments, a target site of a gRNA may be referred to as a protospacer.

Provided herein are gRNAs, such as gRNAs that target or bind to a target site, such as any described herein. In some embodiments, the gRNA is capable of complexing with the Cas protein or variant thereof. In some embodiments, the gRNA comprises a gRNA spacer sequence (i.e. a spacer sequence or a guide sequence) that is capable of hybridizing to the target site, or that is complementary to the target site. In some embodiments, the gRNA comprises a scaffold sequence that complexes with or binds to the Cas protein. In some embodiments, a gRNA specific to a target locus of interest (e.g. comprising a target site) is used to recruit an RNA-guided protein (e.g. a Cas protein) or variant thereof or a fusion protein comprising such RNA-guided protein (e.g., a Cas polypeptide), to the target site.

In some aspects, a “gRNA molecule” is a nucleic acid that promotes the specific targeting or homing of a gRNA molecule/Cas9 molecule complex to a target nucleic acid, such as a locus on the genomic DNA of a cell. gRNA molecules can be unimolecular (having a single RNA molecule), sometimes referred to herein as “chimeric” gRNAs, or modular (comprising more than one, and typically two, separate RNA molecules). In general, a spacer sequence of the guide RNA, is any polynucleotide sequences comprising at least a sequence portion that has sufficient complementarity with a target polynucleotide sequence, such as the at the FXN locus in humans, to hybridize with the target sequence at the target site and direct sequence-specific binding of the CRISPR complex to the target sequence. In some embodiments, in the context of formation of a CRISPR complex, “target sequence” is to a sequence to which a spacer sequence is designed to have complementarity, where hybridization between the target sequence and a spacer sequence of the guide RNA promotes the formation of a CRISPR complex. Full complementarity is not necessarily required, provided there is sufficient complementarity to cause hybridization and promote formation of a CRISPR complex. Generally, a spacer sequence is selected to reduce the degree of secondary structure within the spacer sequence. Secondary structure may be determined by any suitable polynucleotide folding algorithm.

In some embodiments, a guide RNA (gRNA) specific to a target locus of interest (e.g. at the FXN locus in humans) is used with RNA-guided nucleases or variants thereof, e.g., nuclease-inactive Cas variants, to target the provided DNA-targeting system to the target site or target position. Methods for designing gRNAs and exemplary spacer sequences are known. Exemplary gRNA structures that can be associated with particular RNA-guided nucleases or variants thereof, e.g., nuclease-inactive Cas variants, with particular domains and scaffold regions, are also known. In some aspects, gRNA molecules comprise a scaffold sequence, e.g., sequences that can be complexed with the Cas protein. In some aspects, the scaffold sequence is specific for the Cas protein.

In some embodiments, the gRNAs provided herein are chimeric gRNAs. In general, gRNAs can be unimolecular (i.e. composed of a single RNA molecule), or modular (comprising more than one, and typically two, separate RNA molecules). Modular gRNAs can be engineered to be unimolecular, wherein sequences from the separate modular RNA molecules are comprised in a single gRNA molecule, sometimes referred to as a chimeric gRNA, synthetic gRNA, or single gRNA. A guide RNA can comprise at least a spacer sequence that hybridizes to a target nucleic acid sequence of interest, and a CRISPR repeat sequence. In Type II systems, the gRNA also comprises a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex can bind a site-directed polypeptide, such that the guide RNA and site-direct polypeptide form a complex. The gRNA can provide target specificity to the complex by virtue of its association with the site-directed polypeptide. The gRNA thus can direct the activity of the site-directed polypeptide.

In some embodiments, the chimeric gRNA is a fusion of two non-coding RNA sequences: a crRNA sequence and a tracrRNA sequence, for example as described in WO 2013/176772, or Jinek, M. et al. Science 337(6096):816-21 (2012). In some embodiments, the chimeric gRNA mimics the naturally occurring crRNA:tracrRNA duplex involved in the Type II CRISPR/Cas system, wherein the naturally occurring crRNA:tracrRNA duplex acts as a guide for the Cas protein, e.g., Cas9 protein. Exemplary types of CRISPR/Cas systems and associated gRNA structures include those described in, for example, Moon et al. Exp. Mol. Med. 51, 1-11 (2019), Zhang, F. Q. Rev. Biophys. 52, E6 (2019), Makarova et al. Methods Mol. Biol. 1311:47-75 (2015), WO 2013/176772, or Jinek, M. et al. Science 337(6096):816-21 (2012).

In some aspects, the spacer sequence of a gRNA is a polynucleotide sequence comprising at least a portion that has sufficient complementarity with the target site to hybridize with the target site and direct sequence-specific binding of a CRISPR complex to the sequence of the target site. Full complementarity is not necessarily required, provided there is sufficient complementarity to cause hybridization and promote formation of a CRISPR complex. In some embodiments, the gRNA comprises a spacer sequence that is complementary, e.g., at least 80%, 85%, 90%, 95%, 98%, 99%, or 100% (e.g., fully complementary), to the target site. The strand of the target nucleic acid comprising the target site sequence may be referred to as the “complementary strand” of the target nucleic acid. In some aspects, the spacer sequence is a user-defined sequence. Guidance on the selection of spacer sequences can be found, e.g., in Fu et al., Nat Biotechnol 2014 (32:279-284) and Sternberg et al., Nature 2014 507:62-67.

In some embodiments, the gRNA spacer sequence is between about 14 nt and about 26 nt, between about 14 nt and about 24 nt, or between 16 nt and 22 nt in length. In some embodiments, the gRNA spacer sequence is 14 nt, 15 nt, 16 nt, 17 nt, 18 nt, 19 nt, 20 nt, 21 nt or 22 nt, 23 nt, 24 nt, 25 nt, or 26 nt in length. In some embodiments, the gRNA spacer sequence is 18 nt, 19 nt, 20 nt, 21 nt or 22 nt in length. In some embodiments, the gRNA spacer sequence is 18 nt in length. In some embodiments, the gRNA spacer sequence is 19 nt in length. In some embodiments, the gRNA spacer sequence is 20 nt in length. In some embodiments, the gRNA spacer sequence is 21 nt in length. In some embodiments, the gRNA spacer sequence is 22 nt in length.

Methods for designing gRNAs and exemplary targeting domains can include those described in, e.g., International PCT Pub. Nos. WO 2014/197748, WO 2016/130600, WO 2017/180915, WO 2021/226555, WO 2013/176772, WO 2014/152432, WO 2014/093661, WO 2014/093655, WO 2015/089427, WO 2016/049258, WO 2016/123578, WO 2021/076744, WO 2014/191128, WO 2015/161276, WO 2017/193107, and WO 2017/093969.

A target site of a gRNA may be referred to as a protospacer. In some aspects, the spacer is designed to target a protospacer (i.e. target site) with a specific protospacer-adjacent motif (PAM), i.e. a sequence immediately adjacent to the protospacer that contributes to and/or is required for Cas binding specificity. Different CRISPR/Cas systems have different PAM requirements for targeting. For example, S. pyogenes Cas9 uses the PAM 5′-NGG-3′ (SEQ ID NO:50), where N is any nucleotide; S. aureus Cas9 uses the PAM 5′-NNGRRT-3′ (SEQ ID NO:51), where N is any nucleotide, and R is G or A; N. meningitidis Cas9 uses the PAM 5′-NNNNGATT−3′ (SEQ ID NO:52), where N is any nucleotide; C. jejuni Cas9 uses the PAM 5′-NNNNRYAC-3′, (SEQ ID NO:53) or 5′-NNNNACAC-3′(SEQ ID NO:226), where N is any nucleotide, R is G or A, and Y is C or T; S. thermophilus uses the PAM 5′-NNAGAAW-3′ (SEQ ID NO:54), where N is any nucleotide and W is A or T; F. Novicida Cas9 uses the PAM 5′-NGG-3′ (SEQ ID NO:50), where N is any nucleotide; T. denticola Cas9 uses the PAM 5′-NAAAAC-3′ (SEQ ID NO:55), where N is any nucleotide; Cas12a (also known as Cpf1) uses the PAM 5′-TTTV-3′ (SEQ ID NO:56), where V is A, C, or G. Phage-derived CasPhi (such as CasPhi-2, also known as Cas12j), uses the PAM 5′-TBN-3′ (SEQ ID NO:304), where N is any nucleotide, and B is G, T, or C. Archaeal Un1Cas12f1 (also known as Cas14a1), uses the PAM 5′-TTTN−3′ (SEQ ID NO:305), where N is any nucleotide. A Cas12f protein (also known as Cas14) uses the PAM 5′-TTTR−3′ (SEQ ID NO:308), where R is G or A. A Cas12k,protein uses the PAM 5′-GGTT−3′ (SEQ ID NO:307). Cas proteins may use or be engineered to target sequences having different PAMs from those listed above. For example, variant SpCas9 proteins may use sequences having a PAM selected from: 5′-NGG-3′ (SEQ ID NO:50), 5′-NGAN-3′ (SEQ ID NO:57), 5′-NGNG-3′(SEQ ID NO:58), 5′-NGAG-3′(SEQ ID NO:59), or 5′-NGCG-3′(SEQ ID NO:60), where N is any nucleotide. Methods for designing or identifying gRNA spacer sequences and/or protospacer sequences in a particular region, are known. gRNA spacer sequences and/or protospacer sequences can be determined based on the type of Cas protein used and the associated PAM sequence.

In some embodiments, the PAM of a gRNA for complexing with S. pyogenes Cas9 or variant thereof is set forth in SEQ ID NO:NO:50. In some embodiments, the PAM of a gRNA for complexing with S. aureus Cas9 or variant thereof is set forth in SEQ ID NO:NO:51. In some embodiments, the PAM of a gRNA for complexing with a Type V CRISPR/Cas system, such as with Cas12a (also known as Cpf1) or variant thereof is set forth in SEQ ID NO:56.

A spacer sequence may be selected to reduce the degree of secondary structure within the spacer sequence. Secondary structure may be determined by any suitable polynucleotide folding algorithm.

In some embodiments, the gRNA (including the spacer sequence) will comprise the base uracil (U), whereas DNA encoding the gRNA molecule will comprise the base thymine (T). While not wishing to be bound by theory, in some embodiments, it is believed that the complementarity of the spacer sequence (i.e. guide sequence) with the target sequence contributes to specificity of the interaction of the gRNA molecule/Cas molecule complex with a target nucleic acid. It is understood that in a spacer sequence (i.e. guide sequence) and target sequence pair, the uracil bases in the spacer sequence (i.e. guide sequence) will pair with the adenine bases in the target sequence. A gRNA spacer sequence herein may be defined by the DNA sequence encoding the gRNA spacer, and/or the RNA sequence of the spacer.

In some embodiments, the gRNA comprises modified nucleotides, e.g. for increased stability. In some embodiments, one, more than one, or all of the nucleotides of a gRNA can have a modification, e.g., to render the gRNA less susceptible to degradation and/or improve bio-compatibility. By way of non-limiting example, the backbone of the gRNA can be modified with a phosphorothioate, or other modification(s). In some cases, a nucleotide of the gRNA can comprise a 2′ modification, e.g., a 2-acetylation, e.g., a 2′ methylation, or other modification(s).

In some embodiments the gRNA is a concatenation of two non-coding RNA sequences: a crRNA sequence and a tracrRNA sequence. The gRNA may target a desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II CRISPR/Cas system (e.g., Cas9). This duplex, which may include, for example, a 42-nucleotide crRNA and a 75-nucleotide tracrRNA, acts as a guide for the Cas9 protein to cleave the target nucleic acid. The “target region”, “target sequence” or “protospacer” as used interchangeably herein refers to the region of the target gene to which the CRISPR/Cas9-based system targets. The CRISPR/Cas9-based system may include two or more gRNAs, wherein the two or more gRNAs target different DNA sequences. The target DNA sequences may be overlapping or non-overlapping. The target DNA sequences may be located within or near the same gene or different genes. The target sequence or protospacer is followed by a PAM sequence at the 3′ end of the protospacer. Different Type II systems have differing PAM requirements. For example, the Streptococcus pyogenes Type II system uses an “NGG” sequence, where “N” can be any nucleotide.

In some aspects, the gRNA can target the DNA-targeting system to direct the activities of an associated polypeptide (e.g., fusion protein, DNA-targeting system, effector domain, etc.) to a specific target site within a target nucleic acid or a target locus.

In some embodiments, a gRNA provided herein targets a target site, such as any target site described herein. In some embodiments, the gRNA targets a target site for a target gene, such as for any target gene described herein. In some embodiments the gRNA hybridizes to the sequence complementary to the sequence defined as the target site. The strand of the target nucleic acid comprising the target site sequence may be referred to as the “complementary strand” of the target nucleic acid.

In some embodiments, the gRNA targets a target site for a FXN gene. In some embodiments, the gRNA targets a FXN gene or regulatory element thereof, such as an enhancer or promoter. In some aspects, provided herein is a guide RNA (gRNA) that binds a target site in an enhancer region of a frataxin (FXN) locus, wherein the target site is located within the genomic coordinates human genome assembly GRCh38 (hg38) chr9:69,027,282-69,028,497. In some aspects, provided herein is a guide RNA (gRNA) that binds a target site in an enhancer region of a frataxin (FXN) locus, wherein the target site is located within the genomic coordinates hg38 chr9:69,027,615-69,028,101.

In some embodiments, the gRNA targets a target site that comprises a sequence selected from any one of SEQ ID NOS:208-228, as shown in Table 4, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of any one of SEQ ID NOS:208-228 that is 14, 15, 16, 17, 18, 19, 20, or 21 nucleotides in length. In some embodiments, the target site is set forth in any one of SEQ ID NOS:208-228.

In some embodiments, the gRNA comprises a spacer sequence selected from any one of SEQ ID NOS:229-249, as shown in Table 4, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of any one of SEQ ID NOS:229-249 that is 14, 15, 16, 17, 18, 19, 20, or 21 nucleotides in length. In some embodiments, the spacer sequence of the gRNA is set forth in any one of SEQ ID NOS:229-249.

In some embodiments, the gRNA targets a target site that comprises a sequence selected from any one of SEQ ID NOS:319-326, 374, and 375, as shown in Table 4, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of any one of SEQ ID NOS:319-326, 374, and 375 that is 14, 15, 16, 17, 18, 19, 20, or 21 nucleotides in length. In some embodiments, the spacer sequence of the gRNA is set forth in any one of SEQ ID NOS:319-326, 374, and 375.

In some embodiments, the gRNA comprises a spacer sequence selected from any one of SEQ ID NOS:319-326, 374, and 375, as shown in Table 4, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of any one of SEQ ID NOS:319-326, 374, and 375 that is 14, 15, 16, 17, 18, 19, 20, or 21 nucleotides in length. In some embodiments, the spacer sequence of the gRNA is set forth in any one of SEQ ID NOS:319-326, 374, and 375.

In some embodiments, the gRNA targets a target site that comprises a sequence selected from any one of SEQ ID NOS:347-373, as shown in Table 4, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of any one of SEQ ID NOS:347-373 that is 14, 15, 16, 17, 18, 19, 20, or 21 nucleotides in length. In some embodiments, the spacer sequence of the gRNA is set forth in any one of SEQ ID NOS:347-373.

In some embodiments, the gRNA comprises a spacer sequence selected from any one of SEQ ID NOS:347-373, as shown in Table 4, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of any one of SEQ ID NOS:347-373 that is 14, 15, 16, 17, 18, 19, 20, or 21 nucleotides in length. In some embodiments, the spacer sequence of the gRNA is set forth in any one of SEQ ID NOS:347-373.

In some embodiments, any of the provided gRNA sequences is complexed with or is provided in combination with a Cas protein or a variant thereof. In some embodiments, any of the provided gRNA sequences is complexed with or is provided in combination with a Cas9. In some embodiments, the Cas9 is a dCas9. In some embodiments, the dCas9 is a dSaCas9, such as a dSaCas9 set forth in SEQ ID NO:2, or a variant and/or fusion thereof. In some embodiments, the dCas9 is a dSpCas9, such as a dSpCas9 set forth in SEQ ID NO:6, or a variant and/or fusion thereof. In some embodiments, any of the provided gRNA sequences is complexed with or is provided in combination with a Cas12a (also known as Cpf1). In some embodiments, the Cas12a is a dCas12a. In some embodiments, the dCas12a is a dSaCas12a, such as a dSaCas12a set forth in SEQ ID NO:328, or a variant and/or fusion thereof.

In some aspects, the gRNA comprises scaffold sequences. In some aspects, the scaffold sequence (in some cases including a crRNA sequence and/or a tracrRNA sequence) will be different depending on the Cas protein. In some aspects, different CRISPR/Cas systems have different gRNA scaffold sequences for associating with Cas protein.

In some embodiments, the gRNA further comprises a scaffold sequence. In some embodiments, the scaffold sequence is a SaCas9 scaffold sequence. In some embodiments, an exemplary scaffold sequence for S. aureus Cas9 comprises a sequence set forth in SEQ ID NO:169, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:169. In some embodiments, an exemplary scaffold sequence for S. aureus Cas9 comprises a sequence set forth in SEQ ID NO:169. In some embodiments, the scaffold sequence is a SpCas9 scaffold sequence. In some embodiments, an exemplary scaffold sequence for S. pyogenes Cas9 comprises a sequence set forth in SEQ ID NO:171, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:171. In some embodiments, an exemplary scaffold sequence for S. pyogenes Cas9 comprises a sequence set forth in SEQ ID NO:171. In some embodiments, an exemplary scaffold sequence for Acidaminococcus sp. Cas12a comprises a sequence set forth in SEQ ID NO:311, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:311. In some embodiments, an exemplary scaffold sequence for CasPhi-2 comprises a sequence set forth in SEQ ID NO:312, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:312. In some embodiments, an exemplary scaffold sequence for Un1Cas12f1 comprises a sequence set forth in SEQ ID NO:313, 314 or 315, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:313, 314 or 315. In some embodiments, an exemplary scaffold sequence for Un1Cas12f1 comprises a sequence set forth in SEQ ID NO:313, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:313. In some embodiments, an exemplary scaffold sequence for Un1Cas12f1 comprises a sequence set forth in SEQ ID NO:314, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:314. In some embodiments, an exemplary scaffold sequence for Un1Cas12f1 comprises a sequence set forth in SEQ ID NO:315, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:315. In some embodiments, an exemplary scaffold sequence for C. jejuni Cas9 comprises a sequence set forth in SEQ ID NO:316, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:316. In some embodiments, an exemplary scaffold sequence for Cas12k comprises a sequence set forth in SEQ ID NO:317, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:317. In some embodiments, an exemplary scaffold sequence for CasMini comprises a sequence set forth in SEQ ID NO:318, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:318.

In some embodiments, a gRNA provided herein comprises a spacer sequence selected from any one of SEQ ID NOS:229-238 and 249. In some embodiments, the gRNA further comprises a SaCas9 scaffold sequence set forth in SEQ ID NO:169. In some embodiments, the gRNA comprises the sequence selected from any one of SEQ ID NOS:254-263 and 274, as shown in Table 5, or a sequence having at or at least 80%, 85%, 90%, 91%, 92%, 93%, 94%, 95%, 96%, 97%, 98%, 99%, 99.5%, 99.9%, or 100% sequence identity to any one of SEQ ID NOS:254-263 and 274. In some embodiments, the gRNA is set forth in any one of SEQ ID NOS:254-263 and 274. In some embodiments, the gRNA is used with a DNA-targeting domain and/or fusion protein that comprises a dSaCas9, such as a dSaCas9 set forth in SEQ ID NO:2, or a variant and/or fusion thereof.

In some embodiments, a gRNA provided herein comprises a spacer sequence selected from any one of SEQ ID NOS:239-248. In some embodiments, the gRNA further comprises a SpCas9 scaffold sequence set forth in SEQ ID NO:171. In some embodiments, the gRNA comprises the sequence selected from any one of SEQ ID NOS:264-273, as shown in Table 5, or a sequence having at or at least 80%, 85%, 90%, 91%, 92%, 93%, 94%, 95%, 96%, 97%, 98%, 99%, 99.5%, 99.9%, or 100% sequence identity to any one of SEQ ID NOS:264-273. In some embodiments, the gRNA is set forth in any one of SEQ ID NOS:264-273. In some embodiments, the gRNA is used with a DNA-targeting domain and/or fusion protein that comprises a dSpCas9, such as a dSpCas9 set forth in SEQ ID NO:6, or a variant and/or fusion thereof.

In some embodiments, a gRNA provided herein comprises a spacer sequence selected from any one of SEQ ID NOS:319-326, 374, and 375. In some embodiments, the gRNA further comprises a SpCas9 scaffold sequence set forth in SEQ ID NO:171. In some embodiments, the gRNA is used with a DNA-targeting domain and/or fusion protein that comprises a dSpCas9, such as a dSpCas9 set forth in SEQ ID NO:6, or a variant and/or fusion thereof.

In some embodiments, a gRNA provided herein comprises a spacer sequence selected from any one of SEQ ID NOS:347-373. In some embodiments, the gRNA further comprises a SpCas9 scaffold sequence set forth in SEQ ID NO:311. In some embodiments, the gRNA is used with a DNA-targeting domain and/or fusion protein that comprises a dAsCas12a, such as a dAsCas12a set forth in SEQ ID NO:328, or a variant and/or fusion thereof.

TABLE 4
Genes, target site sequences, and gRNA spacer sequences

| | Target sequence | | | gRNA | Scaffold / |
|---|---|---|---|---|---|
| | (e.g. | Target | gRNA spacer | spacer | compatible |
| Gene | protospacer) | SEQ ID | sequence | SEQ ID | Cas9 |
| FXN | TCACACAGCTAGGAA | 208 | UCACACAGCUAGG | 229 | SaCas9 |
| | GTGGG | | AAGUGGG | | |
| FXN | GAGGCTGCTTGGCCG | 209 | GAGGCUGCUUGGC | 230 | SaCas9 |
| | CCGGT | | CGCCGGU | | |
| FXN | GCCCGCTCCGCCCTCC | 210 | GCCCGCUCCGCCCU | 231 | SaCas9 |
| | AGCG | | CCAGCG | | |
| FXN | AGCCTGCTTTGTGCAA | 211 | AGCCUGCUUUGUG | 232 | SaCas9 |
| | AGCA | | CAAAGCA | | |
| FXN | CCGGCCGATGACGCG | 212 | CCGGCCGAUGACG | 233 | SaCas9 |
| | CCGCGGG | | CGCCGCGGG | | |
| FXN | GGCCGGCTACTGCGC | 213 | GGCCGGCUACUGC | 234 | SaCas9 |
| | GGCGCCC | | GCGGCGCCC | | |
| FXN | ACCTCTAGCTGCTCCC | 214 | ACCUCUAGCUGCU | 235 | SaCas9 |
| | CCACAG | | CCCCCACAG | | |
| FXN | CCGCCCGCTCCGCCCT | 215 | CCGCCCGCUCCGCC | 236 | SaCas9 |
| | CCAGCG | | CUCCAGCG | | |
| FXN | GCCCGAGAGTCCACA | 216 | GCCCGAGAGUCCA | 237 | SaCas9 |
| | TGCTGCT | | CAUGCUGCU | | |
| FXN | GCTCTCCATTTTTGTT | 217 | GCUCUCCAUUUUU | 238 | SaCas9 |
| | AAATGC | | GUUAAAUGC | | |
| FXN | CTGCTGTAAACCCATA | 218 | CUGCUGUAAACCC | 239 | SpCas9 |
| | CCGG | | AUACCGG | | |
| FXN | GCAGAGTACAGATTT | 219 | GCAGAGUACAGAU | 240 | SpCas9 |
| | ACACA | | UUACACA | | |
| FXN | CAAGGGAGACTGCAG | 220 | CAAGGGAGACUGC | 241 | SpCas9 |
| | CCTGG | | AGCCUGG | | |
| FXN | AAGCTGGGAAGTTCT | 221 | AAGCUGGGAAGUU | 242 | SpCas9 |
| | TCCTG | | CUUCCUG | | |
| FXN | ATGCACGAATAGTGC | 222 | AUGCACGAAUAGU | 243 | SpCas9 |
| | TAAGC | | GCUAAGC | | |
| FXN | TACACAAGGCATCCG | 223 | UACACAAGGCAUC | 244 | SpCas9 |
| | TCTCC | | CGUCUCC | | |
| FXN | GGCTGCTTGGCCGCC | 224 | GGCUGCUUGGCCG | 245 | SpCas9 |
| | GGTAT | | CCGGUAU | | |
| FXN | TTTAGAAGCGGCGGG | 225 | UUUAGAAGCGGCG | 246 | SpCas9 |
| | CCACC | | GGCCACC | | |
| FXN | AATTGAGGCTGCTTG | 226 | AAUUGAGGCUGCU | 247 | SpCas9 |
| | GCCGC | | UGGCCGC | | |
| FXN | GCAAAGCACGGAGTG | 227 | GCAAAGCACGGAG | 248 | SpCas9 |
| | CAACC | | UGCAACC | | |
| FXN | TGCATGGGACAACCA | 228 | UGCAUGGGACAAC | 249 | SaCas9 |
| | CCACGA | | CACCACGA | | |
| FXN | AAGTAAGGCAAAAAG | 319 | | | SpCas9 |
| | ATTGC | | | | |
| FXN | CAATTTTACTCCTTAG | 320 | | | SpCas9 |
| | GGGA | | | | |
| FXN | GAGAGCAGATACTAC | 321 | | | SpCas9 |
| | TGCCA | | | | |
| FXN | TGGAGACCAAGAATG | 322 | | | SpCas9 |
| | CTGCT | | | | |
| FXN | TGATTTTGTGATCTTG | 323 | | | SpCas9 |
| | CGAA | | | | |
| FXN | TAACATGCTGTACAG | 324 | | | SpCas9 |
| | GTTTA | | | | |
| FXN | CTAGACCATCTAGCTT | 325 | | | SpCas9 |
| | TGTG | | | | |
| FXN | TCTCCACCCACTAGAT | 326 | | | SpCas9 |
| | GGCA | | | | |
| FXN | TCCCCTAAGGAGTAA | 374 | | | SpCas9 |
| | AATTG | | | | |
| FXN | TCTAACTTTGAATTAA | 375 | | | SpCas9 |
| | ATAT | | | | |
| FXN | TGGAAGTAAGGCAAA | 347 | | | AsCas12a |
| | AAGATTGC | | | | |
| FXN | AGGCAAAAAGATTGC | 348 | | | AsCas12a |
| | TTTGAATA | | | | |
| FXN | AGTGGTTCTCAACCA | 349 | | | AsCas12a |
| | GGGGCAAT | | | | |
| FXN | CAATTTTACTCCTTAG | 350 | | | AsCas12a |
| | GGGACCT | | | | |
| FXN | TTTTCACAATGTCTGG | 351 | | | AsCas12a |
| | AGACATT | | | | |
| FXN | TGGGAAATGCATCAT | 352 | | | AsCas12a |
| | TAGGTGAT | | | | |
| FXN | AATTGTACTACAGTA | 353 | | | AsCas12a |
| | GTTAAGTA | | | | |
| FXN | AGTATGGTAACATGC | 354 | | | AsCas12a |
| | TGTACAGG | | | | |
| FXN | TATAGTAGGCTAGAC | 355 | | | AsCas12a |
| | CATCTAGC | | | | |
| FXN | TCACAAAATCACCTA | 356 | | | AsCas12a |
| | ATGATGCA | | | | |
| FXN | TTGTCCCATGCATTGT | 357 | | | AsCas12a |
| | AAGGTGG | | | | |
| FXN | GAGAACCACTATTCT | 358 | | | AsCas12a |
| | GGTCTAAC | | | | |
| FXN | AATTAAATATTCAAA | 359 | | | AsCas12a |
| | GCAATCTT | | | | |
| FXN | ATCTTTTTGCCTTACT | 360 | | | AsCas12a |
| | TCCAAGT | | | | |
| FXN | CTTACTTCCAAGTTTT | 361 | | | AsCas12a |
| | AGATAAT | | | | |
| FXN | GCTCACGCCTGTCGTC | 362 | | | AsCas12a |
| | TCAGCAC | | | | |
| FXN | GTGAGGCCCAGGCGG | 363 | | | AsCas12a |
| | GCAGATCG | | | | |
| FXN | CAGCAGCTCCCAAGT | 364 | | | AsCas12a |
| | TCCTCCTG | | | | |
| FXN | CCCAAGTTCCTCCTGT | 365 | | | AsCas12a |
| | TTAGAAT | | | | |
| FXN | TAATATTTTCAAGGCT | 366 | | | AsCas12a |
| | GGATTTT | | | | |
| FXN | CTGGATTTTTTTGAAC | 367 | | | AsCas12a |
| | GAAATGC | | | | |
| FXN | TAGCTATTCTGCAGCT | 368 | | | AsCas12a |
| | CTGAAAG | | | | |
| FXN | TTTCACCTCGTTCCAG | 369 | | | AsCas12a |
| | GAAAGCA | | | | |
| FXN | TCACTGCAAGCTCTGC | 370 | | | AsCas12a |
| | CTCCCGG | | | | |
| FXN | CCGCCACCATGCCTG | 371 | | | AsCas12a |
| | GCTAATTT | | | | |
| FXN | ACACCCAGCTAGTTTT | 372 | | | AsCas12a |
| | TGTATTT | | | | |
| FXN | TTGTATTTTTTGTAGA | 373 | | | AsCas12a |
| | AAGGGGG | | | | |

TABLE 5
Genes and gene-targeting gRNAs

| | | gRNA | |
|---|---|---|---|
| Target | | SEQ | compatible |
| gene | gRNA sequence | ID | Cas9 |
| FXN | UCACACAGCUAGGAAGUGGGGUUUUAGUACUCUGGAAACA | 254 | SaCas9 |
| | GAAUCUACUAAAACAAGGCAAAAUGCCGUGUUUAUCUCGU | | |
| | CAACUUGUUGGCGAGA | | |
| FXN | GAGGCUGCUUGGCCGCCGGUGUUUUAGUACUCUGGAAACA | 255 | SaCas9 |
| | GAAUCUACUAAAACAAGGCAAAAUGCCGUGUUUAUCUCGU | | |
| | CAACUUGUUGGCGAGA | | |
| FXN | GCCCGCUCCGCCCUCCAGCGGUUUUAGUACUCUGGAAACAG | 256 | SaCas9 |
| | AAUCUACUAAAACAAGGCAAAAUGCCGUGUUUAUCUCGUC | | |
| | AACUUGUUGGCGAGA | | |
| FXN | AGCCUGCUUUGUGCAAAGCAGUUUUAGUACUCUGGAAACA | 257 | SaCas9 |
| | GAAUCUACUAAAACAAGGCAAAAUGCCGUGUUUAUCUCGU | | |
| | CAACUUGUUGGCGAGA | | |
| FXN | CCGGCCGAUGACGCGCCGCGGGGUUUUAGUACUCUGGAAA | 258 | SaCas9 |
| | CAGAAUCUACUAAAACAAGGCAAAAUGCCGUGUUUAUCUC | | |
| | GUCAACUUGUUGGCGAGA | | |
| FXN | GGCCGGCUACUGCGCGGCGCCCGUUUUAGUACUCUGGAAA | 259 | SaCas9 |
| | CAGAAUCUACUAAAACAAGGCAAAAUGCCGUGUUUAUCUC | | |
| | GUCAACUUGUUGGCGAGA | | |
| FXN | ACCUCUAGCUGCUCCCCCACAGGUUUUAGUACUCUGGAAAC | 260 | SaCas9 |
| | AGAAUCUACUAAAACAAGGCAAAAUGCCGUGUUUAUCUCG | | |
| | UCAACUUGUUGGCGAGA | | |
| FXN | CCGCCCGCUCCGCCCUCCAGCGGUUUUAGUACUCUGGAAAC | 261 | SaCas9 |
| | AGAAUCUACUAAAACAAGGCAAAAUGCCGUGUUUAUCUCG | | |
| | UCAACUUGUUGGCGAGA | | |
| FXN | GCCCGAGAGUCCACAUGCUGCUGUUUUAGUACUCUGGAAA | 262 | SaCas9 |
| | CAGAAUCUACUAAAACAAGGCAAAAUGCCGUGUUUAUCUC | | |
| | GUCAACUUGUUGGCGAGA | | |
| FXN | GCUCUCCAUUUUUGUUAAAUGCGUUUUAGUACUCUGGAAA | 263 | SaCas9 |
| | CAGAAUCUACUAAAACAAGGCAAAAUGCCGUGUUUAUCUC | | |
| | GUCAACUUGUUGGCGAGA | | |
| FXN | CUGCUGUAAACCCAUACCGGGUUUAAGAGCUAUGCUGGAA | 264 | SpCas9 |
| | ACAGCAUAGCAAGUUUAAAUAAGGCUAGUCCGUUAUCAAC | | |
| | UUGAAAAAGUGGCACCGAGUCGGUGC | | |
| FXN | GCAGAGUACAGAUUUACACAGUUUAAGAGCUAUGCUGGAA | 265 | SpCas9 |
| | ACAGCAUAGCAAGUUUAAAUAAGGCUAGUCCGUUAUCAAC | | |
| | UUGAAAAAGUGGCACCGAGUCGGUGC | | |
| FXN | CAAGGGAGACUGCAGCCUGGGUUUAAGAGCUAUGCUGGAA | 266 | SpCas9 |
| | ACAGCAUAGCAAGUUUAAAUAAGGCUAGUCCGUUAUCAAC | | |
| | UUGAAAAAGUGGCACCGAGUCGGUGC | | |
| FXN | AAGCUGGGAAGUUCUUCCUGGUUUAAGAGCUAUGCUGGAA | 267 | SpCas9 |
| | ACAGCAUAGCAAGUUUAAAUAAGGCUAGUCCGUUAUCAAC | | |
| | UUGAAAAAGUGGCACCGAGUCGGUGC | | |
| FXN | AUGCACGAAUAGUGCUAAGCGUUUAAGAGCUAUGCUGGAA | 268 | SpCas9 |
| | ACAGCAUAGCAAGUUUAAAUAAGGCUAGUCCGUUAUCAAC | | |
| | UUGAAAAAGUGGCACCGAGUCGGUGC | | |
| FXN | UACACAAGGCAUCCGUCUCCGUUUAAGAGCUAUGCUGGAA | 269 | SpCas9 |
| | ACAGCAUAGCAAGUUUAAAUAAGGCUAGUCCGUUAUCAAC | | |
| | UUGAAAAAGUGGCACCGAGUCGGUGC | | |
| FXN | GGCUGCUUGGCCGCCGGUAUGUUUAAGAGCUAUGCUGGAA | 270 | SpCas9 |
| | ACAGCAUAGCAAGUUUAAAUAAGGCUAGUCCGUUAUCAAC | | |
| | UUGAAAAAGUGGCACCGAGUCGGUGC | | |
| FXN | UUUAGAAGCGGCGGGCCACCGUUUAAGAGCUAUGCUGGAA | 271 | SpCas9 |
| | ACAGCAUAGCAAGUUUAAAUAAGGCUAGUCCGUUAUCAAC | | |
| | UUGAAAAAGUGGCACCGAGUCGGUGC | | |
| FXN | AAUUGAGGCUGCUUGGCCGCGUUUAAGAGCUAUGCUGGAA | 272 | SpCas9 |
| | ACAGCAUAGCAAGUUUAAAUAAGGCUAGUCCGUUAUCAAC | | |
| | UUGAAAAAGUGGCACCGAGUCGGUGC | | |
| FXN | GCAAAGCACGGAGUGCAACCGUUUAAGAGCUAUGCUGGAA | 273 | SpCas9 |
| | ACAGCAUAGCAAGUUUAAAUAAGGCUAGUCCGUUAUCAAC | | |
| | UUGAAAAAGUGGCACCGAGUCGGUGC | | |
| FXN | UGCAUGGGACAACCACCACGAGUUUUAGUACUCUGGAAAC | 274 | SaCas9 |
| | AGAAUCUACUAAAACAAGGCAAAAUGCCGUGUUUAUCUCG | | |
| | UCAACUUGUUGGCGAGA | | |

### B. Other DNA-Targeting Domains

In some of any of the provided embodiments, the DNA-targeting domain comprises a zinc finger protein (ZFP); a transcription activator-like effector (TALE); a meganuclease; a homing endonuclease; or an I-SceI enzyme or a variant thereof. In some embodiments, the DNA-targeting domain binds to the target site, e.g. at the endogenous locus and/or for the target gene. In some embodiments, the DNA-targeting domain comprises a catalytically inactive variant of any of the foregoing.

In some embodiments, the DNA-targeting domain comprises a zinc finger protein (ZFP), i.e. is a ZFP-based DNA-targeting domain. In some embodiments, a zinc finger protein (ZFP), a zinc finger DNA binding protein, or zinc finger DNA binding domain, is a protein, or a domain within a larger protein, that binds DNA in a sequence-specific manner through one or more zinc fingers, which are regions of amino acid sequence within the binding domain, having a structure that is stabilized through coordination of a zinc ion. The term zinc finger DNA binding protein is often abbreviated as zinc finger protein or ZFP. Among the ZFPs are artificial, or engineered, ZFPs, comprising ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is engineered to bind to a target site of choice.

In some cases, the DNA-targeting system is or comprises a zinc-finger DNA binding domain fused to an effector domain. In some embodiments, zinc fingers are custom-designed (i.e. designed by the user), or obtained from a commercial source. Various methods for designing zinc finger proteins are available. For example, methods for designing zinc finger proteins to bind to a target DNA sequence of interest are described, for example in Liu, Q. et al., PNAS, 94(11):5525-30 (1997); Wright, D. A. et al., Nat. Protoc., 1(3):1637-52 (2006); Gersbach, C. A. et al., Acc. Chem. Res., 47(8):2309-18 (2014); Bhakta M. S. et al., Methods Mol. Biol., 649:3-30 (2010); and Gaj et al., Trends Biotechnol, 31(7):397-405 (2013). In addition, various web-based tools for designing zinc finger proteins to bind to a DNA target sequence of interest are publicly available. See, for example, the Zinc Finger Tools design web site from Scripps available on the world wide web at scripps.edu/barbas/zfdesign/zfdesignhome.php. Various commercial services for designing zinc finger proteins to bind to a DNA target sequence of interest are also available. See, for example, the commercially available services or kits offered by Creative Biolabs (world wide web at creative-biolabs.com/Design-and-Synthesis-of-Artificial-Zinc-Finger-Proteins.html), the Zinc Finger Consortium Modular Assembly Kit available from Addgene (world wide web at addgene.org/kits/zfc-modular-assembly/), or the CompoZr Custom ZFN Service from Sigma Aldrich (world wide web at sigmaaldrich.com/life-science/zinc-finger-nuclease-technology/custom-zfn.html). For example, platforms for zinc-finger construction are available that provide specifically targeted zinc fingers for thousands of targets. See, e.g., Gaj et al., Trends in Biotechnology, 2013, 31(7), 397-405. Some gene-specific engineered zinc fingers are available commercially. In some cases, commercially available zinc fingers are used or are custom designed.

In some embodiments, the DNA-targeting domain is based on transcription activator-like effectors (TALEs), i.e. is a TALE-based DNA-targeting domain. TALEs are proteins naturally found in Xanthomonas bacteria. TALEs comprise a plurality of repeated amino acid sequences, each repeat having binding specificity for one base in a target sequence. Each repeat comprises a pair of variable residues in position 12 and 13 (repeat variable diresidue; RVD) that determine the nucleotide specificity of the repeat. In some embodiments, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In some embodiments, RVDs can be mutated towards other amino acid residues in order to modulate their specificity towards nucleotides A, T, C and G and in particular to enhance this specificity. Binding domains with similar modular base-per-base nucleic acid binding properties can also be derived from different bacterial species. These alternative modular proteins may exhibit more sequence variability than TALE repeats.

In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205.

In some embodiments, a TALE is a fusion protein comprising a nucleic acid binding domain derived from a TALE and an effector domain. In some embodiments, one or more sites in an endogenous locus can be targeted by engineered TALEs.

ZFP and TALE-based DNA-targeting domains can be engineered to bind to a predetermined nucleotide sequence, for example via engineering (altering one or more amino acids) of the recognition helix region of a naturally occurring zinc finger protein, by engineering of the amino acids in a TALE repeat involved in DNA binding (the repeat variable diresidue or RVD region), or by systematic ordering of modular DNA-targeting domains, such as TALE repeats or ZFP domains. Therefore, engineered ZFP or TALE proteins are proteins that are non-naturally occurring. Non-limiting examples of methods for engineering ZFPs and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

### IV. Fusion Proteins

In some aspects, provided herein are fusion proteins. In some embodiments, the fusion protein comprises any of the transcriptional activation domains and/or multipartite effector such as multipartite activator described herein. In some embodiments, the fusion protein comprises a DNA-targeting domain or component thereof described herein. In some embodiments, the fusion protein is capable of targeting the transcriptional activation domains and/or multipartite effector such as multipartite activator to a target site for a target gene. In some embodiments, the fusion protein is capable of being targeted to a target site for a target gene, by virtue of the DNA-targeting domain or component thereof. In some aspects, targeting of the transcriptional activation domains and/or multipartite effector such as multipartite activators or the fusion protein increases transcription of the target gene. In some aspects, the fusion protein is comprised in a DNA-targeting system, for example, in combination with a guide RNA (gRNA).

In some embodiments, the fusion protein comprises (a) a DNA-targeting domain or a component thereof, such as any described herein, and (b) any of the transcriptional activation domains and/or multipartite effector for transcriptional activation, such as any multipartite activator described herein. In some embodiments, the fusion protein comprises (a) any of the DNA-targeting domain or component thereof described herein, such as any described in Section III, and (b) any of the transcriptional activation domain or multipartite effector such as multipartite activator described herein, such as any described in Section I.

In some aspects, the fusion protein comprises a DNA-targeting domain or a component thereof, and (b) two or more transcriptional activation domains. In some embodiments, the transcriptional activation domain comprises any of the transcriptional activation domains provided herein, such as any described in Section I.A, a portion thereof, a partially or fully functional fragment or domain thereof, or a combination of any of the foregoing. In some embodiments, each transcriptional activation domain comprises a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. In some embodiments, one or more transcriptional activation domain comprises a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. In some embodiments, one or more transcriptional activation domain comprises any of the transcriptional activation domains provided herein, such as any described in Section I.A, including VP64, p65, Rta, p300, CBP, VPR, VPH, HSF1, a TET protein (e.g. TET1), a partially or fully functional fragment or domain thereof, or a combination of any of the foregoing. In some embodiments, the fusion protein comprises (a) a DNA-targeting domain or component thereof, and (b) a multipartite effector for transcriptional activation, such as any multipartite activator described herein, for example in Section I.B. In some embodiments, the DNA-targeting domain is a ZFN, TALE, or CRISPR/Cas-based DNA-targeting domain. In some embodiments, the DNA-targeting domain targets the fusion protein to a target site for a target gene, thereby increasing transcription of (i.e. activating) the target gene. In some aspects, the fusion protein is targeted to a target site for a gene and leads to increased transcription of the gene.

In some embodiments, any two or more domains of the fusion protein are heterologous, i.e. the domains are from different species, or at least one of the domains is not found in nature. In some aspects, the fusion protein is an engineered fusion protein, i.e. the fusion protein is not found in nature.

In some embodiments, the fusion protein comprises its constituent components (e.g. domains) in any suitable arrangement, orientation, or order. For example, a transcriptional activation domain may be fused to the N-terminus or C-terminus of the DNA-targeting domain of the fusion protein, and/or to the N-terminus or C-terminus of another transcriptional activation domain or multipartite effector such as multipartite activator of the fusion protein. In some embodiments, any two components of the fusion protein may be fused directly (i.e. without an intervening amino acid sequence). In some embodiments, any two components of the fusion protein may be fused indirectly, e.g. via an intervening amino acid sequence, such as a linker or nuclear localization signal (NLS), such as any linker or NLS described herein.

In some embodiments, the fusion protein comprises one or more linkers. In some embodiments, the one or more linkers connect any two components of the fusion protein. A linker may be included anywhere in the polypeptide sequence of the fusion protein, for example, between a transcriptional activation domain and the DNA-targeting domain or a component thereof. A linker may be of any length and designed to promote or restrict the mobility of components in the fusion protein. In some embodiments, inclusion of a linker in the fusion protein enhances the function of the fusion protein. For example, inclusion of the linker in the fusion protein may lead to enhanced activation of the target gene in comparison to a comparable fusion protein without the linker.

A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, 30, 35, 40, 45, 50, 55, 60, 65, 70, 75, 80 or 85 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may be rich in amino acids glycine (G), serine (S), and/or alanine (A). Linkers may include, for example, a GS linker. An exemplary GS linker is represented by the sequence GGGGS (SEQ ID NO:67). A linker may comprise repeats of a sequence, for example as represented by the formula (GGGGS)n, wherein n is an integer that represents the number of times the GGGGS sequence is repeated (e.g. between 1 and 10 times). The number of times a linker sequence is repeated can be adjusted to optimize the linker length and achieve appropriate separation of the functional domains. Other examples of linkers may include, for example, GGGGG (SEQ ID NO:63), GGAGG (SEQ ID NO:64), GGGGSSS (SEQ ID NO:65), or GGGGAAA (SEQ ID NO:66), or GSGSG (SEQ ID NO:206).

In some embodiments, the linker is an XTEN linker. In some aspects, an XTEN linker is a recombinant polypeptide (e.g., an unstructured recombinant peptide) lacking hydrophobic amino acid residues. Exemplary XTEN linkers are described in, for example, Schellenberger et al., Nature Biotechnology 27, 1186-1190 (2009) or WO 2021/247570. In some embodiments, the linker comprises a linker described in WO 2021/247570. In some aspects, the linker is or comprises the sequence set forth in SEQ ID NO:96 or SEQ ID NO:174, or a portion thereof, or an amino acid sequence that has at least 90%, 91%, 92%, 93%, 94%, 95%, 96%, 97%, 98%, or 99% sequence identity to any of the foregoing. In some embodiments, the linker comprises the sequence set forth in SEQ ID NO:96, or a portion thereof, or an amino acid sequence that has at least 90%, 91%, 92%, 93%, 94%, 95%, 96%, 97%, 98%, or 99% sequence identity to any of the foregoing. In some aspects, the linker comprises the sequence set forth in SEQ ID NO:96, or a contiguous portion of SEQ ID NO:96 of at least 5, 10, 15, 20, 25, 30, 35, 40, 45, 50, 55, 60, 65, 70 or 75 amino acids. In some aspects, the linker consists of the sequence set forth in SEQ ID NO:96, or a contiguous portion of SEQ ID NO:96 of at least 5, 10, 15, 20, 25, 30, 35, 40, 45, 50, 55, 60, 65, 70 or 75 amino acids. In some embodiments, the linker comprises the sequence set forth in SEQ ID NO:96. In some embodiments, the linker consist of the sequence set forth in SEQ ID NO:96. In some embodiments, the linker comprises the sequence set forth in SEQ ID NO:174, or a portion thereof, or an amino acid sequence that has at least 90%, 91%, 92%, 93%, 94%, 95%, 96%, 97%, 98%, or 99% sequence identity to any of the foregoing. In some aspects, the linker comprises the sequence set forth in SEQ ID NO:174, or a contiguous portion of SEQ ID NO:174 of at least 5, 10, or 15 amino acids. In some aspects, the linker consists of the sequence set forth in SEQ ID NO:174, or a contiguous portion of SEQ ID NO:174 of at least 5, 10, or 15 amino acids. In some embodiments, the linker comprises the sequence set forth in SEQ ID NO:174. In some embodiments, the linker consist of the sequence set forth in SEQ ID NO:174. Appropriate linkers may be selected or designed based rational criteria known in the art, for example as described in Chen et al. Adv. Drug Deliv. Rev. 65(10):1357-1369 (2013).

In some embodiments, the fusion protein comprises one or more nuclear localization signal (NLS). In some embodiments, a fusion protein described herein comprises one or more nuclear localization sequences (NLSs), such as about or more than about 1, 2, 3, 4, 5, 6, 7, 8, 9, 10, or more NLSs. When more than one NLS is present, each may be selected independently of the others, such that a single NLS may be present in more than one copy and/or in combination with one or more other NLSs present in one or more copies. Non-limiting examples of NLSs include an NLS sequence derived from: the NLS of the SV40 virus large T-antigen, having the amino acid sequence PKKKRKV (SEQ ID NO:70); the NLS from nucleoplasmin (e.g. the nucleoplasmin bipartite NLS with the sequence KRPAATKKAGQAKKKK (SEQ ID NO:69)); the c-myc NLS having the amino acid sequence PAAKRVKLD (SEQ ID NO:71) or RQRRNELKRSP (SEQ ID NO:72); the hRNPA1 M9 NLS having the sequence NQSSNFGPMKGGNFGGRSSGPYGGGGQYFAKPRNQGGY (SEQ ID NO:73); the sequence RMRIZFKNKGKDTAELRRRRVEVSVELRKAKKDEQILKRRNV (SEQ ID NO:74) of the IBB domain from importin-alpha; the sequences VSRKRPRP (SEQ ID NO:75) and PPKKARED (SEQ ID NO:76) of the myoma T protein; the sequence PQPKKKPL (SEQ ID NO:77) of human p53; the sequence SALIKKKKKMAP (SEQ ID NO:78) of mouse c-abl IV; the sequences DRLRR (SEQ ID NO:79) and PKQKKRK (SEQ ID NO:80) of the influenza virus NS1; the sequence RKLKKKIKKL (SEQ ID NO:81) of the Hepatitis virus delta antigen; the sequence REKKKFLKRR (SEQ ID NO:82) of the mouse Mx1 protein; the sequence KRKGDEVDGVDEVAKKKSKK (SEQ ID NO:83) of the human poly(ADP-ribose) polymerase; and the sequence RKCLQAGMNLEARKTKK (SEQ ID NO:84) of the steroid hormone receptors (human) glucocorticoid. The NLS may comprise a portion of any of the foregoing. In some embodiments, the one or more NLSs are of sufficient strength to drive localization and/or accumulation of the fusion protein in the nucleus of a eukaryotic cell, such as in a detectable amount. In some embodiments, the strength of nuclear localization activity may derive from the number of NLSs in the fusion protein, the particular NLS(s) used, or a combination of these factors. Detection of accumulation in the nucleus may be performed by any suitable technique. For example, a detectable marker may be fused to the fusion protein, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of the fusion protein (e.g. an assay for altered gene expression activity in a cell transformed with the fusion protein), as compared to a control condition (e.g. an untransformed cell).

In some aspects, the DNA-targeting system or fusion protein comprises one or more tags, linkers and/or NLS sequences. In some embodiments, exemplary tags, linkers and/or NLS sequences can be any described herein.

In some cases, sequences provided herein, including amino acid sequences for the DNA-targeting systems or fusion proteins provided herein, contain sequences of one or more tags, linkers and/or NLS sequences. In some aspects, it is understood that the exemplary tags, linkers and/or NLS sequences are not required or are not the sole or exclusive tags, linkers and/or NLS sequences that can be employed in the DNA-targeting systems or fusion proteins. In some aspects, sequences containing tags, linkers and/or NLS sequences are exemplary, and are not limited to the specific tags, linkers and/or NLS sequences contained in the described sequences. In some aspects, alternative tags, linkers and/or NLS sequences can be can be employed in the DNA-targeting systems or fusion proteins, or the DNA-targeting system or fusion protein in some cases does not contain or lacks a tag, linker and/or NLS. In some aspects, alternative tags, linkers and/or NLS sequences include other known tags, linkers and/or NLS sequences that have similar function or serve similar purposes.

In some embodiments, the fusion protein is a split protein, i.e. comprises two or more separate polypeptide domains that interact or self-assemble to form a functional fusion protein. In some embodiments, the split fusion protein is assembled from separate polypeptide domains comprising trans-splicing inteins. Inteins are internal protein elements that self-excise from their host protein and catalyze ligation of flanking sequences with a peptide bond. In some embodiments, the split fusion protein is assembled from a first polypeptide comprising an N-terminal intein and a second polypeptide comprising a C-terminal intein. In some embodiments, the N terminal intein is the N terminal Npu Intein set forth in SEQ ID NO:88. In some embodiments, the C terminal intein is the C terminal Npu intein set forth in SEQ ID NO:92.

Also provided are fusion proteins comprising a first polypeptide of a split variant Cas protein comprising an N-terminal fragment of a Cas protein and an N-terminal Intein, and any of the multipartite effector domains provided herein. Also provided are fusion proteins comprising a first polypeptide of a split variant Cas protein comprising an N-terminal fragment of a Cas protein and an N-terminal Intein, and any of the multipartite effector domains provided herein, wherein the multipartite effector domain increases transcription of a target locus. In some aspects, the first polypeptide of the split variant Cas protein, and a second polypeptide of the split variant Cas protein comprising a C-terminal fragment of the variant Cas protein and a C-terminal Intein, are present in proximity or present in the same cell, the N-terminal Intein and C-terminal Intein self-excise and ligate the N-terminal fragment and the C-terminal fragment of the variant Cas9 to form a full-length variant Cas9 protein.

Also provided are fusion proteins comprising a second polypeptide of a split variant Cas protein comprising a C-terminal fragment of a Cas protein and a C-terminal Intein and any of the multipartite effector domains provided herein. Also provided are fusion proteins comprising a second polypeptide of a split variant Cas protein comprising a C-terminal fragment of a Cas protein and a C-terminal Intein and any of the multipartite effector domains provided herein, wherein the multipartite effector domain increases transcription of a target locus. In some aspects, the second polypeptide of the split variant Cas protein, and a first polypeptide of the split variant Cas protein comprising an N-terminal fragment of the variant Cas protein and an N-terminal Intein, are present in proximity or present in the same cell, the N-terminal Intein and C-terminal Intein self-excise and ligate the N-terminal fragment and the C-terminal fragment of the variant Cas9 to form a full-length variant Cas9 protein.

In some embodiments, the split fusion protein comprises a split dCas9, such as a split dSpCas9. In an exemplary embodiment, a first polypeptide comprises an N-terminal fragment of dSpCas9, followed by an N terminal Npu Intein, and a second polypeptide comprises a C terminal Npu Intein, followed by a C-terminal fragment of dSpCas9. In some embodiments, the N- and C-terminal fragments of the fusion protein are split at position 573Glu of the SpCas9 molecule, with reference to SEQ ID NO:7.

In some aspects, the N-terminal Npu Intein and C-terminal Npu Intein may self-excise and ligate the two fragments, thereby forming the full-length dSpCas9 fusion protein when expressed in a cell.

In some embodiments, the polypeptides of a split protein may interact non-covalently to form a complex that recapitulates the activity of the non-split protein. For example, two domains of a Cas enzyme expressed as separate polypeptides may be recruited by a gRNA to form a ternary complex that recapitulates the activity of the full-length Cas enzyme in complex with the gRNA, for example as described in Wright et al. PNAS 112(10):2984-2989 (2015). In some embodiments, assembly of the split protein is inducible (e.g. light inducible, chemically inducible, small-molecule inducible).

In some aspects, the two polypeptides of a split fusion protein may be delivered and/or expressed from separate vectors, such as any of the vectors described herein. In some embodiments, the two polypeptides of a split fusion protein may be delivered to a cell and/or expressed from two separate AAV vectors, i.e. using a split AAV-based approach, for example as described in WO 2017/197238.

Approaches for the rationale design of split proteins and their delivery, including Cas proteins and fusions thereof, are described, for example, in WO 2016/114972, WO 2017/197238, Zetsche. et al. Nat. Biotechnol. 33(2):139-42 (2015), Wright et al. PNAS 112(10):2984-2989 (2015), Truong. et al. Nucleic Acids Res. 43, 6450-6458 (2015), and Fine et al. Sci. Rep. 5, 10777 (2015).

In some embodiments, the fusion protein comprises dCas9 and a multipartite effector such as multipartite activator comprising domains from FOXO3 and NCOA3, respectively. In some embodiments, the dCas is dSaCas9 or dSpCas9. In some embodiments, the fusion protein is dSaCas9-FOXO3-NCOA3. In some embodiments, the fusion protein comprises SEQ ID NO:180 or 275, or an amino acid sequence that has at least 90%, 91%, 92%, 93%, 94%, 95%, 96%, 97%, 98%, or 99% sequence identity to SEQ ID NO:180 or 275. In some embodiments, the fusion protein sequence is set forth in SEQ ID NO: 180. In some embodiments, the fusion protein sequence is set forth in SEQ ID NO:275.

In some embodiments, the fusion protein comprises dCas9 and a multipartite effector such as multipartite activator comprising domains from PYGO1, FOXO3, and NCOA3, respectively. In some embodiments, the dCas is dSaCas9 or dSpCas9. In some embodiments, the fusion protein is dSaCas9-PYGO1-FOXO3-NCOA3. In some embodiments, the fusion protein comprises SEQ ID NO:181 or 276, or an amino acid sequence that has at least 90%, 91%, 92%, 93%, 94%, 95%, 96%, 97%, 98%, or 99% sequence identity to SEQ ID NO:181 or 276. In some embodiments, the fusion protein sequence is set forth in SEQ ID NO:181. In some embodiments, the fusion protein sequence is set forth in SEQ ID NO:276.

In some embodiments, the fusion protein comprises dCas9 and a multipartite effector such as multipartite activator comprising domains from NOTCH2, FOXO3, and NCOA3, respectively. In some embodiments, the dCas is dSaCas9 or dSpCas9. In some embodiments, the fusion protein is dSaCas9-NOTCH2-FOXO3-NCOA3. In some embodiments, the fusion protein comprises SEQ ID NO:182 or 277, or an amino acid sequence that has at least 90%, 91%, 92%, 93%, 94%, 95%, 96%, 97%, 98%, or 99% sequence identity to SEQ ID NO:182 or 277. In some embodiments, the fusion protein sequence is set forth in SEQ ID NO:182. In some embodiments, the fusion protein sequence is set forth in SEQ ID NO:277.

In some embodiments, the fusion protein comprises dCas9 and a multipartite effector such as multipartite activator comprising domains from NCOA3, FOXO3, and NCOA3, respectively. In some embodiments, the dCas is dSaCas9 or dSpCas9. In some embodiments, the fusion protein is dSaCas9-NCOA3-FOXO3-NCOA3. In some embodiments, the fusion protein comprises SEQ ID NO:183 or 278, or an amino acid sequence that has at least 90%, 91%, 92%, 93%, 94%, 95%, 96%, 97%, 98%, or 99% sequence identity to SEQ ID NO:183 or 278. In some embodiments, the fusion protein sequence is set forth in SEQ ID NO:183. In some embodiments, the fusion protein sequence is set forth in SEQ ID NO:278.

In some embodiments, the fusion protein comprises dCas9 and a multipartite effector such as multipartite activator comprising domains from HSH2D, FOXO3, and NCOA3, respectively. In some embodiments, the dCas is dSaCas9 or dSpCas9. In some embodiments, the fusion protein is dSaCas9-HSH2D-FOXO3-NCOA3. In some embodiments, the fusion protein comprises SEQ ID NO:184 or 279, or an amino acid sequence that has at least 90%, 91%, 92%, 93%, 94%, 95%, 96%, 97%, 98%, or 99% sequence identity to SEQ ID NO:184 or 279. In some embodiments, the fusion protein sequence is set forth in SEQ ID NO:184. In some embodiments, the fusion protein sequence is set forth in SEQ ID NO:279.

In some embodiments, the fusion protein comprises dCas9 and a multipartite effector such as multipartite activator comprising domains from FOXO3, FOXO3, and NCOA3, respectively. In some embodiments, the dCas is dSaCas9 or dSpCas9. In some embodiments, the fusion protein is dSaCas9-FOXO3-FOXO3-NCOA3. In some embodiments, the fusion protein comprises SEQ ID NO:185 or 280, or an amino acid sequence that has at least 90%, 91%, 92%, 93%, 94%, 95%, 96%, 97%, 98%, or 99% sequence identity to SEQ ID NO:185 or 280. In some embodiments, the fusion protein sequence is set forth in SEQ ID NO:185. In some embodiments, the fusion protein sequence is set forth in SEQ ID NO:280.

In some embodiments, the fusion protein comprises dCas9 and a multipartite effector such as multipartite activator comprising domains from NCOA2, FOXO3, and NCOA3, respectively. In some embodiments, the dCas is dSaCas9 or dSpCas9. In some embodiments, the fusion protein is dSaCas9-NCOA2-FOXO3-NCOA3. In some embodiments, the fusion protein comprises SEQ ID NO:186 or 281, or an amino acid sequence that has at least 90%, 91%, 92%, 93%, 94%, 95%, 96%, 97%, 98%, or 99% sequence identity to SEQ ID NO:186 or 281. In some embodiments, the fusion protein sequence is set forth in SEQ ID NO:186. In some embodiments, the fusion protein sequence is set forth in SEQ ID NO:281.

In some embodiments, the fusion protein comprises dCas9 and a multipartite effector such as multipartite activator comprising domains from ENL, FOXO3, and NCOA3, respectively. In some embodiments, the dCas is dSaCas9 or dSpCas9. In some embodiments, the fusion protein is dSaCas9-ENL-FOXO3-NCOA3. In some embodiments, the fusion protein comprises SEQ ID NO:187 or 282, or an amino acid sequence that has at least 90%, 91%, 92%, 93%, 94%, 95%, 96%, 97%, 98%, or 99% sequence identity to SEQ ID NO:187 or 282. In some embodiments, the fusion protein sequence is set forth in SEQ ID NO: 187. In some embodiments, the fusion protein sequence is set forth in SEQ ID NO:282.

### V. DNA-Targeting Systems

In some embodiments, provided herein are DNA-targeting systems. In some embodiments, the DNA-targeting system comprises any of the transcriptional activation domains, multipartite effector such as multipartite activators, DNA-targeting domains, and/or fusion proteins described herein. In some aspects, the DNA-targeting system comprises a guide RNA (gRNA). In some aspects, the DNA-targeting system is capable of specifically targeting a target site, such as any target site described herein. In some aspects, the DNA-targeting system recruits two or more transcriptional activation domains and/or a multipartite effector such as multipartite activator for transcriptional activation to the target site, thereby increasing transcription of the target gene. In some aspects, the DNA-targeting system comprises a DNA-targeting domain that is capable of targeting, such as binding or hybridizing to, the target site. In some embodiments, the target site is any target site provided herein.

In some embodiments, the DNA-targeting system is a CRISPR/Cas-based DNA-targeting system (i.e. comprises one or more CRISPR/Cas-based DNA-targeting domains), a ZFN-based DNA-targeting system (i.e. comprises one or more ZFN-based DNA-targeting domains), or a TALE-based DNA-targeting system (i.e. comprises one or more TALE-based DNA-targeting domains). In some embodiments, the DNA-targeting system comprises different types of DNA-targeting domains, for example a DNA-targeting system comprising a CRISPR/Cas-based DNA-targeting domain and a ZFN-based DNA-targeting domain. In some aspects, the DNA-targeting system comprises a fusion protein comprising two or more transcriptional activation domain, and a DNA-targeting domain such as a CRISPR/Cas-based DNA-targeting domain, and one or more gRNAs.

In some embodiments, the DNA-targeting system comprises a fusion protein comprising a DNA-targeting domain, such as a ZFN-based or TALE-based DNA-targeting domain, and two or more transcriptional activation domains, each independently selected from NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. In some embodiments, the DNA-targeting system comprises a fusion protein comprising a DNA-targeting domain, such as a ZFN-based or TALE-based DNA-targeting domain, and a multipartite effector such as multipartite activator described herein.

In some embodiments, the DNA-targeting system comprises (a) a fusion protein comprising a dCas protein (e.g. dCas9), and two or more transcriptional activation domains, each independently selected from NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2, and (b) at least one gRNA. In some embodiments, the DNA-targeting system comprises (a) a fusion protein comprising a Cas protein (such as dCas9) and a multipartite effector such as multipartite activator described herein, and (b) at least one gRNA.

In some embodiments, the DNA-targeting system targets a single target site for a target gene. In some embodiments, the DNA-targeting system targets multiple target sites, such as two or more target sites. In some embodiments, two or more of the multiple target sites are for the same target gene (i.e. the targeting is combinatorial). In some aspects, targeting multiple sites for the same gene may lead to increased, or prolonged, transcriptional activation of the gene, in comparison to targeting a single target site for the gene. In some embodiments, two or more of the multiple target sites are for different genes (i.e. the targeting is multiplexed). In some aspects, targeting target sites for two or more different genes can lead to increased transcription of the two or more different genes. In some aspects, increased transcription of two or more different genes results in or promotes a cellular phenotype.

In some aspects, DNA-targeting systems targeting two or more target sites may comprise two or more DNA-targeting modules, each DNA-targeting module comprising a portion of the DNA-targeting system that targets one target site. For example, an individual DNA-targeting module may comprise a fusion protein comprising a DNA-targeting domain (e.g. a ZFN or TALE-based DNA-targeting domain) that targets a target site, and two or more transcriptional activation domains. In some embodiments, a DNA-targeting module comprises (a) a fusion protein comprising a Cas protein and two or more transcriptional activation domains, and (b) a gRNA that targets the Cas protein (and thereby the fusion protein) to the target site. A DNA-targeting system provided herein may comprise one or more (such as two) DNA-targeting modules.

In some embodiments, two DNA-targeting modules of a DNA-targeting system may comprise separate, (i.e. non-overlapping) components. For example, a DNA-targeting system may comprise two different fusion proteins, each fusion protein targeting and activating a different gene. For example, the DNA-targeting system may comprise a first DNA-targeting module comprising a first fusion protein comprising a DNA-targeting domain that targets a first target site, and a second DNA-targeting module comprising a second fusion protein comprising a DNA-targeting domain that targets a second target site. In another example, the DNA-targeting system may comprise a first DNA-targeting module comprising a first fusion protein comprising a DNA-targeting domain that targets a first target site, and a second DNA-targeting module comprising (a) a fusion protein comprising a dCas protein and (b) a gRNA that targets the dCas protein to a second target site. In another example, the DNA-targeting system may comprise a first DNA-targeting module comprising a first fusion protein comprising a first dCas protein and (b) a first gRNA that complexes with the first Cas protein and targets a first target site, and a second DNA-targeting module comprising a second fusion protein comprising a second dCas protein that is different from the first dCas protein, and (b) a second gRNA that complexes with the second dCas protein and targets the second dCas protein to a second target site. It will be understood that different Cas protein variants (e.g. SpCas9 and SaCas9) complex (i.e. are compatible) with different gRNA scaffold sequences and PAMs, as described herein. Thus, it is possible to engineer a single DNA-targeting system comprising multiple non-overlapping CRISPR/Cas-based DNA-targeting modules, each targeting a different target site.

In some embodiments, two DNA-targeting modules of a DNA-targeting system may comprise shared (i.e. overlapping) components. For example, a DNA-targeting system may comprise a first DNA-targeting module comprising (a) a fusion protein comprising a Cas protein, and (b) a gRNA that targets a target site of a first gene, and a second DNA-targeting module comprising (a) the fusion protein of the first DNA-targeting module, and (b) a gRNA that targets a target site of a second gene. It will be understood that providing two or more different gRNAs for a given Cas protein allows different molecules of the same Cas protein to be targeted to the target sites of the two or more gRNAs.

In some embodiments, the DNA-targeting system comprises a SunTag system. SunTag is a system for recruiting multiple copies of a protein to a polypeptide scaffold. In the SunTag system, the polypeptide scaffold comprises a repeating array of GCN4 peptides (peptides forming epitopes derived from the S. cerevisiae GCN4 gene), which recruits multiple copies of an antibody-fusion protein comprising scFv(GCN4), a single-chain variable fragment antibody that binds the repeated GCN4 peptide. The antibody-fusion protein may comprise an effector domain, such as a transcriptional activation domain. The repeating peptide array can be fused to a DNA-targeting domain (e.g. a CRISPR/Cas-based DNA-targeting domain), such that the effector domain is recruited to the target sequence. SunTag, including in dCas fusion proteins for gene activation, has been described, for example, in WO 2016/011070 and Tanenbaum, M. et al. Cell. 159(3):635-646 (2014).

In some embodiments, a DNA-targeting system provided herein comprises (a) a fusion protein comprising a DNA-targeting domain or component thereof and a SunTag repeating peptide array, and (b) an antibody-fusion protein comprising scFv(GCN4) and any of the transcriptional activation domains provided herein, such as two or more transcriptional activation domains selected from DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, and NOTCH2, and/or any of the multipartite effector such as any multipartite activators described herein. In some embodiments, the DNA-targeting system comprises (a) a fusion protein comprising dCas9 and a SunTag repeating peptide array, (b) an antibody-fusion protein comprising scFv(GCN4) and any of the transcriptional activation domains provided herein, such as two or more transcriptional activation domains selected from DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, and NOTCH2, and/or any of the multipartite effector such as any multipartite activators described herein, and (c) a gRNA. In some embodiments, the DNA-targeting system comprises (a) a fusion protein comprising a ZFN-based or TALE-based DNA-targeting domain and a SunTag repeating peptide array, and (b) an antibody-fusion protein comprising scFv(GCN4) and any of the transcriptional activation domains provided herein, such as two or more transcriptional activation domains selected from DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, and NOTCH2. In some embodiments, the SunTag repeating peptide array comprises a repeating GCN4 peptide having the amino acid sequence LLPKNYHLENEVARLKKLVGER (SEQ ID NO:61) separated by linkers comprising the sequence GGSGG (SEQ ID NO:62). Thus, the repeating peptide array may comprise two or more tandem copies of the amino acid sequence LLPKNYHLENEVARLKKLVGERGGSGG (SEQ ID NO:204). In some embodiments, the DNA-targeting system comprises an optimized version of the SunTag repeating peptide array. In some embodiments, the repeating peptide array comprises a repeating GCN4 peptide having the amino acid sequence EELLSKNYHLENEVARLKK (SEQ ID NO:205), separated by linkers comprising the sequence GSGSG (SEQ ID NO:206). Thus, the repeating peptide array may comprise two or more tandem copies of the amino acid sequence EELLSKNYHLENEVARLKKGSGSG (SEQ ID NO:207).

### VI. Polynucleotides, Vectors, and Methods for Delivery

In some aspects, provided are polynucleotides encoding any of the transcriptional activation domains, multipartite effectors such as any multipartite activators, DNA-targeting domains, gRNAs, fusion proteins, or DNA-targeting systems described herein, or a portion, component, or combination of any of the foregoing. In some aspects, the polynucleotides can encode any of the components of the aforementioned compositions, and/or any nucleic acid or proteinaceous molecule necessary to carry out aspects of the methods of the disclosure.

In some embodiments, provided are polynucleotides comprising the gRNAs described herein. In some embodiments, the gRNA is transcribed from a genetic construct (i.e. vector or plasmid) in the target cell. In some embodiments, the gRNA is produced by in vitro transcription and delivered to the target cell. In some embodiments, the gRNA comprises one or more modified nucleotides for increased stability. In some embodiments, the gRNA is delivered to the target cell pre-complexed as a RNP with the fusion protein.

In some embodiments, a provided polynucleotide encodes a fusion protein as described herein that comprises two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2. In some embodiments, the fusion protein further comprises a DNA-targeting domain (e.g. a ZFN-based or TALE-based DNA-targeting domain), or component thereof (e.g. a dCas protein of a CRISPR/Cas-based DNA-targeting domain). Examples of such domains and fusion proteins include any as described herein.

In some embodiments, the polynucleotide encodes an amino acid sequence set forth in any of SEQ ID NOS:140-160 and 180-187, or a sequence having at least about 80%, 85%, 90%, 91%, 92%, 93%, 94%, 95%, 96%, 97%, 98%, 99%, or 100% sequence identity thereto. In some embodiments, the polynucleotide encodes the amino acid sequence set forth in any one of SEQ ID NO:140-160 and 180-187.

In some embodiments, the polynucleotide comprises the sequence set forth in any of SEQ ID NOS:109-129, or a sequence having at least about 80%, 85%, 90%, 91%, 92%, 93%, 94%, 95%, 96%, 97%, 98%, 99%, or 100% sequence identity thereto. In some embodiments, the polynucleotide comprises the sequence set forth in any of SEQ ID NOS:109-129. In some embodiments, the polynucleotide comprises SEQ ID NO:109. In some embodiments, the polynucleotide comprises SEQ ID NO:110. In some embodiments, the polynucleotide comprises SEQ ID NO:111. In some embodiments, the polynucleotide comprises SEQ ID NO:112. In some embodiments, the polynucleotide comprises SEQ ID NO:113. In some embodiments, the polynucleotide comprises SEQ ID NO:114. In some embodiments, the polynucleotide comprises SEQ ID NO:115. In some embodiments, the polynucleotide comprises SEQ ID NO:116. In some embodiments, the polynucleotide comprises SEQ ID NO:117. In some embodiments, the polynucleotide comprises SEQ ID NO:118. In some embodiments, the polynucleotide comprises SEQ ID NO:119. In some embodiments, the polynucleotide comprises SEQ ID NO:120. In some embodiments, the polynucleotide comprises SEQ ID NO:121. In some embodiments, the polynucleotide comprises SEQ ID NO:122. In some embodiments, the polynucleotide comprises SEQ ID NO:123. In some embodiments, the polynucleotide comprises SEQ ID NO:124. In some embodiments, the polynucleotide comprises SEQ ID NO:125. In some embodiments, the polynucleotide comprises SEQ ID NO:126. In some embodiments, the polynucleotide comprises SEQ ID NO:127. In some embodiments, the polynucleotide comprises SEQ ID NO:128. In some embodiments, the polynucleotide comprises SEQ ID NO:129.

In some embodiments, the polynucleotide is RNA or DNA. In some embodiments, the polynucleotide, such as a polynucleotide encoding a provided fusion protein, is mRNA. In some embodiments, the gRNA is provided as RNA and a polynucleotide encoding the fusion protein is mRNA. The mRNA can be 5′ capped and/or 3′ polyadenylated. In another embodiment, a polynucleotide provided herein, such as a polynucleotide encoding a provided fusion protein, is DNA. The DNA can be present in a vector.

Also provided herein is a vector that contains any of the provided polynucleotides. In some embodiments, the vector comprises a genetic construct, such as a plasmid or an expression vector.

In some embodiments, an expression vector comprising a sequence encoding a fusion protein comprising a dCas may further comprise a polynucleotide sequence encoding at least one gRNA. In some embodiments, the expression vector comprises a polynucleotide sequence or combination of polynucleotide sequences encoding two gRNAs. In some embodiments, the expression vector comprises a polynucleotide sequence or combination of polynucleotide sequences encoding three gRNAs. The sequence encoding the gRNA can be operably linked to at least one transcriptional control sequence for expression of the gRNA in the cell. For example, DNA encoding the gRNA can be operably linked to a promoter sequence that is recognized by RNA polymerase III (Pol III). Examples of suitable Pol III promoters include, but are not limited to, mammalian U6, U3, H1, and 7SL RNA promoters.

In some embodiments, provided is a vector containing (a) a polynucleotide that encodes a fusion protein comprising a dCas and two or more transcriptional activation domains and/or a multipartite effector such as multipartite activator, and (b) a polynucleotide or combination of polynucleotides encoding a gRNA, or combination of gRNAs, such as two gRNAs, or three gRNAs. In some embodiments, the dCas is a dCas9, such as dSpCas9 or dSaCas9. In some embodiments, the polynucleotide encodes a fusion protein that includes a dSpCas9 set forth in SEQ ID NO:6, a dSaCas9 set forth in SEQ ID NO:2, or a dAsCas12a set forth in SEQ ID NO:328.

In some embodiments, the polynucleotide as provided herein can be codon optimized for efficient translation into protein in the eukaryotic cell or animal of interest. For example, codons can be optimized for expression in humans, mice, rats, hamsters, cows, pigs, cats, dogs, fish, amphibians, plants, yeast, insects, and so forth. Programs for codon optimization are available as freeware. Commercial codon optimization programs are also available.

In some embodiments, a polynucleotide described herein can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs (gRNAs) used in connection with the DNA-targeting systems, various promoters such as RNA polymerase III promoters, including for example U6 and H1, can be advantageous. Descriptions of and parameters for enhancing the use of such promoters are known in the art, and additional information and approaches include those described in, e.g., Ma, H. et al., Molecular Therapy-Nucleic Acids 3, e161 (2014) doi:10.1038/mtna.2014.12.

The expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, a cell type specific promoter (e.g. nervous system specific promoter), etc.).

In some examples, vectors can be capable of directing the expression of nucleic acids to which they are operatively linked. Such vectors are referred to herein as “recombinant expression vectors”, or more simply “expression vectors”, which serve equivalent functions.

Exemplary expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Other vectors can be used so long as they are compatible with the host cell.

In some embodiments, the vector is a viral vector, such as an adeno-associated virus (AAV) vector, a retroviral vector, a lentiviral vector, or a gammaretroviral vector. n some embodiments, the viral vector is an adeno-associated virus (AAV) vector. In some embodiments, the AAV vector is selected from among an AAV1, AAV2, AAV3, AAV4, AAV5, AAV6, AAV7, AAV8, AAV9, AAV10, AAV11, AAV12, or AAV-DJ vector. In some embodiments, the vector is a lentiviral vector. In some embodiments, the vector is a non-viral vector, for example a lipid nanoparticle, a liposome, an exosome, or a cell penetrating peptide.

In some embodiments, the vector comprises one vector, or two or more vectors.

In some aspects, provided herein are pluralities of vectors that comprise any of the vectors described herein, and one or more additional vectors comprising one or more additional polynucleotides encoding an additional portion or an additional component of any of the DNA-targeting systems described herein, any of the gRNAs described herein, any of the combinations described herein, or any of the fusion proteins described herein, or a portion or a component of any of the foregoing.

Provided are pluralities of vectors, that include: a first vector comprising any of the polynucleotides described herein; and a second vector comprising any of the polynucleotides described herein. Also provided herein are pluralities of vectors, comprising: a first vector comprising a polynucleotide encoding a first DNA-targeting system, a first Cas protein and/or a first gRNA of any of the DNA-targeting system described herein or any of the combinations of gRNAs described herein; and; a second vector comprising a polynucleotide encoding a second DNA-targeting system, a second Cas protein and/or a second gRNA of any of the DNA-targeting system described herein or any of the combinations of gRNAs described herein.

In some embodiments, polynucleotides can be cloned into a suitable vector, such as an expression vector or vectors. The expression vector can be any suitable recombinant expression vector, and can be used to transform or transfect any suitable cell. Suitable vectors include those designed for propagation and expansion or for expression or both, such as plasmids and viruses.

In some embodiments, the vector can be a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some embodiments, animal expression vectors include pEUK-Cl, pMAM and pMAMneo (Clontech). In some embodiments, a viral vector is used, such as a lentiviral or retroviral vector. In some embodiments, the recombinant expression vectors can be prepared using standard recombinant DNA techniques. In some embodiments, vectors can contain regulatory sequences, such as transcription and translation initiation and termination codons, which are specific to the type of host into which the vector is to be introduced, as appropriate and taking into consideration whether the vector is DNA- or RNA-based. In some embodiments, the vector can contain a nonnative promoter operably linked to the nucleotide sequence encoding the recombinant receptor. In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other promoters known to a skilled artisan also are contemplated.

In some embodiments, recombinant nucleic acids are transferred into cells using recombinant infectious virus particles, such as, e.g., vectors derived from simian virus 40 (SV40), adenoviruses, or adeno-associated virus (AAV). In some embodiments, recombinant nucleic acids are transferred into cells (e.g. central nervous system cells, such as neurons, or hepatocytes) using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3: 102-109.

In some embodiments, the vector is a lentiviral vector. In some embodiments, the lentiviral vector is an integrase-deficient lentiviral vector. In some embodiments, the lentiviral vector is a recombinant lentiviral vector. In some embodiments, the lentivirus is selected or engineered for a desired tropism (e.g. for central nervous system tropism, or tropism for a heart cell, such as a cardiomyocyte, a skeletal muscle cell, a nervous system cell, such as a neuron, a fibroblast, for liver cell or hepatocytes, or an induced pluripotent stem cell). In some embodiments, the cell for any of the provided compositions, such as DNA-targeting systems, fusion proteins, gRNAs, polynucleotides and/or vectors to be delivered is a heart cell, a skeletal muscle cell, a nervous system cell, or an induced pluripotent stem cell. Methods of lentiviral production, transduction, and engineering are known, for example as described in Kasaraneni, N. et al. Sci. Rep. 8(1):10990 (2018), Ghaleh, H. E. G. et al. Biomed. Pharmacother. 128:110276 (2020), and Milone, M. C. et al. Leukemia. 32(7):1529-1541 (2018). Additional methods for lentiviral transduction are described, for example in Wang et al. (2012) J. Immunother. 35(9): 689-701; Cooper et al. (2003) Blood. 101: 1637-1644; Verhoeyen et al. (2009) Methods Mol Biol. 506: 97-114; and Cavalieri et al. (2003) Blood. 102(2): 497-505.

In some embodiments, recombinant nucleic acids are transferred into cells (e.g. central nervous system cells, such as neurons, or a heart cell, a skeletal muscle cell, a nervous system cell, hepatocytes, or an induced pluripotent stem cell) via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material into immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

In some embodiments, the viral vector is an AAV vector. In some embodiments, the AAV vector is selected from among an AAV1, AAV2, AAV3, AAV4, AAV5, AAV6, AAV7, AAV8, AAV9, AAV10, AAV11, AAV12, or an AAV-DJ vector. In some embodiments, the AAV vector is an AAV vector engineered for central nervous system (CNS) tropism. In some embodiments, the AAV vector is selected from among an AAV1, AAV2, AAV3, AAV4, AAV5, AAV6, AAV7, AAV8, or AAV9 vector. In some embodiments, the AAV vector is an AAV5 vector or an AAV9 vector. In some aspects, the AAV vector is an AAV9 vector. In some aspects, the AAV vector is an AAV5 vector. In some aspects, the AAV vector is an AAV-DJ vector.

In some embodiments, the AAV is selected or engineered for a desired tropism (e.g. for central nervous system tropism, or tropism for a heart cell, such as a cardiomyocyte, a skeletal muscle cell, a nervous system cell, such as a neuron, a fibroblast, or an induced pluripotent stem cell (iPSC)). In some embodiments, the AAV is exhibits tropism for a cardiomyocyte. In some embodiments, the AAV is exhibits tropism for a nervous system cell. In some embodiments, the AAV is exhibits tropism for a cell of the central nervous system (CNS). In some embodiments, the AAV is exhibits tropism for a neuron. In some embodiments, the AAV is exhibits tropism for a fibroblast. In some embodiments, the AAV is exhibits tropism for an iPSC.

In some aspects, nucleic acids or polynucleotides encoding any of the DNA-targeting systems, guide RNAs, fusion proteins, or components, portions or combinations thereof can be delivered to cells or subjects using gene delivery vectors, such as viral vectors. In some aspects, provided herein are viral vectors that comprise any of the nucleic acids or polynucleotides described herein, any of the pluralities of nucleic acids or polynucleotides described herein, or a first polynucleotide or a second polynucleotide of any of the pluralities of polynucleotides described herein, or a portion or a component of any of the foregoing.

Examples of virions that can be employed to deliver any of the nucleic acids or polynucleotides provided herein include but are not limited to retroviral virions, lentiviral virions, adenovirus virions, herpes virus virions, alphavirus virions, and adeno-associated virus (AAV) virions. AAV is a 4.7 kb, single-stranded DNA virus. Recombinant virions based on AAV (rAAV virions) are associated with excellent clinical safety, since wild-type AAV is nonpathogenic and has no etiologic association with any known diseases. In addition, AAV offers the capability for highly efficient delivery and sustained expression of the delivered nucleic acid, composition or component thereof, in numerous tissues, including the nervous system, eye, muscle, lung and brain.

A “recombinant AAV vector (recombinant adeno-associated viral vector)” in some aspects refers to a polynucleotide vector comprising one or more heterologous sequences (i.e., nucleic acid sequence not of AAV origin) that are flanked by at least one AAV inverted terminal repeat sequences (ITR). In some aspects, the recombinant nucleic acid is flanked by two inverted terminal repeat sequences (ITRs). Such recombinant viral vectors can be replicated and packaged into infectious viral particles when present in a host cell that has been infected with a suitable helper virus (or that is expressing suitable helper functions) and that is expressing AAV rep and cap gene products (i.e., AAV Rep and Cap proteins). When a recombinant viral vector is incorporated into a larger polynucleotide (e.g., in a chromosome or in another vector such as a plasmid used for cloning or transfection), then the recombinant viral vector may be referred to as a “pro-vector” which can be “rescued” by replication and encapsidation in the presence of AAV packaging functions and suitable helper functions. A recombinant viral vector can be in any of a number of forms, including, but not limited to, plasmids, linear artificial chromosomes, complexed with lipids, encapsulated within liposomes, and encapsidated in a viral particle, for example, an AAV particle. A recombinant viral vector can be packaged into an AAV virus capsid to generate a “recombinant adeno-associated viral particle (recombinant viral particle)”.

An “rAAV virus” or “rAAV viral particle” refers to a viral particle composed of at least one AAV capsid protein and an encapsidated rAAV vector genome.

“AAV helper functions” refer to functions that allow AAV to be replicated and packaged by a host cell for producing viruses. AAV helper functions can be provided in any of a number of forms, including, but not limited to, helper virus or helper virus genes which aid in AAV replication and packaging. Other AAV helper functions are known, such as genotoxic agents.

A “helper virus” for AAV refers to a virus that allows AAV (which is a defective parvovirus) to be replicated and packaged by a host cell for producing viruses. A helper virus provides “helper functions” which allow for the replication of AAV. A number of such helper viruses have been identified, including adenoviruses, herpesviruses, poxviruses such as vaccinia and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include E1A functions, E1B functions, E2A functions, VA functions and E4orf6 functions. Baculoviruses available from depositories include Autographa californica nuclear polyhedrosis virus.

A preparation of rAAV is said to be “substantially free” of helper virus if the ratio of infectious AAV particles to infectious helper virus particles is at least about 102:1; at least about 104:1, at least about 106:1; or at least about 108:1 or more. In some aspects, preparations are also free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

In some aspects, the recombinant viral particles for delivery of any of the provided nucleic acids, compositions or components thereof comprise a self-complementary AAV (scAAV) genome. In some aspects, the recombinant AAV genome comprises a first heterologous polynucleotide sequence (e.g., coding strand) and a second heterologous polynucleotide sequence (e.g., the noncoding or antisense strand) wherein the first heterologous polynucleotide sequence can form intrastrand base pairs with the second polynucleotide sequence along most or all of its length. In some aspects, the first heterologous polynucleotide sequence and a second heterologous polynucleotide sequence are linked by a sequence that facilitates intrastrand base-pairing; e.g., a hairpin DNA structure. Hairpin structures are known, for example in siRNA molecules. In some aspects, the first heterologous polynucleotide sequence and a second heterologous polynucleotide sequence are linked by a mutated ITR. In some aspects, the scAAV viral particles comprise a monomeric form of an scAAV genome. In some aspects, the scAAV viral particles comprise the dimeric form of and scAAV genome. In some aspects, AUC as described herein is used to detect the presence of rAAV particles comprising the monomeric form of an scAAV genome. In some aspects, AUC as described herein is used to detect the presence of rAAV particles comprising the dimeric form of an scAAV genome. In some aspects, the packaging of scAAV genomes into capsid is monitored by AUC.

In some aspects, the rAAV particles comprise an AAV1 capsid, an AAV2 capsid, an AAV3 capsid, an AAV4 capsid, an AAV5 capsid, an AAV6 capsid (e.g., a wild-type AAV6 capsid, or a variant AAV6 capsid such as ShH10, as described in US 2012/0164106), an AAV7 capsid, an AAV8 capsid, an AAVrh8 capsid, an AAVrh8R, an AAV9 capsid (e.g., a wild-type AAV9 capsid, or a modified AAV9 capsid as described in US 2013/0323226), an AAV10 capsid, an AAVrh10 capsid, an AAV11 capsid, an AAV12 capsid, a tyrosine capsid mutant, a heparin binding capsid mutant, an AAV2R471A capsid, an AAVAAV2/2-7m8 capsid, an AAV-DJ capsid (e.g., an AAV-DJ/8 capsid, an AAV-DJ/9 capsid, or any other AAV-DJ capsid, such as any of the capsids described, for example, in US 2012/0066783 or Mao, Y. et al., BMC Biotechnol. 16:1 (2016)), an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid, or an AAV capsid described in U.S. Pat. No. 8,283,151 or WO 2003/042397. In some of the above embodiments described herein, the rAAV particles comprise at least one AAV1 ITR, AAV2 ITR, AAV3 ITR, AAV4 ITR, AAV5 ITR, AAV6 ITR, AAV7 ITR, AAV8 ITR, AAVrh8 ITR, AAV9 ITR, AAV10 ITR, AAVrh10 ITR, AAV11 ITR, AAV12 ITR, AAV-DJ ITR, goat AAV ITR, bovine AAV ITR, or mouse AAV ITR. In some aspects, the rAAV particles comprise ITRs from one AAV serotype and AAV capsid from another serotype. For example, the rAAV particles may comprise the nucleic acid to be delivered (e.g., encoding any of the DNA-targeting systems, fusion proteins, gRNA, compositions or components thereof) flanked by at least one AAV2 ITR encapsidated into an AAV9 capsid. Such combinations may be referred to as pseudotyped rAAV particles. Exemplary AAV vectors include those described, for example, in WO 2020/113034, US 20220001028, US 20220001028, US 20210317474, and US 20160097061.

In some aspects, the viral particle is a recombinant AAV particle comprising a nucleic acid to be delivered flanked by one or two ITRs. The nucleic acid is encapsidated in the AAV particle. The AAV particle also comprises capsid proteins. In some aspects, the nucleic acid comprises the protein coding sequence or RNA-expressing sequences to be delivered (e.g., any of the DNA-targeting systems, fusion proteins, gRNA, compositions or components thereof) operatively linked components in the direction of transcription, control sequences including transcription initiation and termination sequences, thereby forming an expression cassette. The expression cassette is flanked on the 5′ and 3′ end by at least one functional AAV ITR sequences. By “functional AAV ITR sequences” it is meant that the ITR sequences function as intended for the rescue, replication and packaging of the AAV virion. See Davidson et al., PNAS, 2000, 97(7)3428-32; Passini et al., J. Virol., 2003, 77(12):7034-40; and Pechan et al., Gene Ther., 2009, 16:10-16, all of which are incorporated herein in their entirety by reference. For practicing some aspects of the invention, the recombinant vectors comprise at least all of the sequences of AAV essential for encapsidation and the physical structures for infection by the rAAV. AAV ITRs for use in the vectors of the invention need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99(18): 11854-6; Gao et al., PNAS, 2003, 100(10):6081-6; and Bossis et al., J. Virol., 2003, 77(12):6799-810. Use of any AAV serotype is considered within the scope of the present invention. In some aspects, a rAAV vector is a vector derived from an AAV serotype, including without limitation, AAV1, AAV2, AAV3, AAV4, AAV5, AAV6, AAV7, AAV8, AAV9, AAVrh.8, AAVrh.10, AAV11, AAV12, a tyrosine capsid mutant, a heparin binding capsid mutant, an AAV2R471A capsid, an AAVAAV2/2-7m8 capsid, an AAV-DJ capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, or a mouse AAV capsid, or the like. In some aspects, the nucleic acid in the AAV comprises an ITR of AAV1, AAV2, AAV3, AAV4, AAV5, AAV6, AAV7, AAV8, AAV9, AAVrh.8, AAVrh10, AAV11, AAV12 or the like. In further embodiments, the rAAV particle comprises capsid proteins of AAV1, AAV2, AAV3, AAV4, AAV5, AAV6, AAV7, AAV8, AAV9, AAVrh.8, AAVrh.10, AAV11, AAV12 or the like. In further embodiments, the rAAV particle comprises capsid proteins of an AAV serotype from Clades A-F (Gao, et al. J. Virol. 2004, 78(12):6381).

Different AAV serotypes are used to optimize transduction of particular target cells or to target specific cell types within a particular target tissue (e.g., a diseased tissue). A rAAV particle can comprise viral proteins and viral nucleic acids of the same serotype or a mixed serotype. For example, a rAAV particle can comprise AAV9 capsid proteins and at least one AAV2 ITR or it can comprise AAV2 capsid proteins and at least one AAV9 ITR. In yet another example, a rAAV particle can comprise capsid proteins from both AAV9 and AAV2, and further comprise at least one AAV2 ITR. Any combination of AAV serotypes for production of a rAAV particle is provided herein as if each combination had been expressly stated herein.

In some aspects, the AAV comprises at least one AAV1 ITR and capsid protein from any of AAV-DJ, AAV2, AAV3, AAV4, AAV5, AAV6, AAV7, AAV8, AAV9, AAVrh.8, AAVrh10, AAV11, and/or AAV12. In some aspects, the AAV comprises at least one AAV2 ITR and capsid protein from any of AAV-DJ, AAV1, AAV3, AAV4, AAV5, AAV6, AAV7, AAV8, AAV9, AAVrh.8, AAVrh10, AAV11, and/or AAV12. In some aspects, the AAV comprises at least one AAV3 ITR and capsid protein from any of AAV-DJ, AAV1, AAV2, AAV4, AAV5, AAV6, AAV7, AAV8, AAV9, AAVrh.8, AAVrh10, AAV11, and/or AAV12. In some aspects, the AAV comprises at least one AAV4 ITR and capsid protein from any of AAV-DJ, AAV1, AAV2, AAV3, AAV5, AAV6, AAV7, AAV8, AAV9, AAVrh.8, AAVrh10, AAV11, and/or AAV12. In some aspects, the AAV comprises at least one AAV5 ITR and capsid protein from any of AAV-DJ, AAV1, AAV2, AAV3, AAV4, AAV6, AAV7, AAV8, AAV9, AAVrh.8, AAVrh10, AAV11, and/or AAV12. In some aspects, the AAV comprises at least one AAV6 ITR and capsid protein from any of AAV-DJ, AAV1, AAV2, AAV3, AAV4, AAV5, AAV7, AAV8, AAV9, AAVrh.8, AAVrh10, AAV11, and/or AAV12. In some aspects, the AAV comprises at least one AAV7 ITR and capsid protein from any of AAV-DJ, AAV1, AAV2, AAV3, AAV4, AAV5, AAV6, AAV8, AAV9, AAVrh.8, AAVrh10, AAV11, and/or AAV12. In some aspects, the AAV comprises at least one AAV8 ITR and capsid protein from any of AAV-DJ, AAV1, AAV2, AAV3, AAV4, AAV5, AAV6, AAV7, AAV9, AAVrh.8, AAVrh10, AAV11, and/or AAV12. In some aspects, the AAV comprises at least one AAV9 ITR and capsid protein from any of AAV-DJ, AAV1, AAV2, AAV3, AAV4, AAV5, AAV6, AAV7, AAV8, AAVrh.8, AAVrh10, AAV11, and/or AAV12. In some aspects, the AAV comprises at least one AAVrh8 ITR and capsid protein from any of AAV-DJ, AAV1, AAV2, AAV3, AAV4, AAV5, AAV6, AAV8, AAV9, AAVrh10, AAV11, and/or AAV12. In some aspects, the AAV comprises at least one AAVrh10 ITR and capsid protein from any of AAV-DJ, AAV1, AAV2, AAV3, AAV4, AAV5, AAV6, AAV7, AAV8, AAV9, AAV11, and/or AAV12. In some aspects, the AAV comprises at least one AAV11 ITR and capsid protein from any of AAV-DJ, AAV1, AAV2, AAV3, AAV4, AAV5, AAV6, AAV7, AAV8, AAVrh8, AAV9, AAVrh10, and/or AAV12. In some aspects, the AAV comprises at least one AAV12 ITR and capsid protein from any of AAV-DJ, AAV1, AAV2, AAV3, AAV4, AAV5, AAV6, AAV7, AAV8, AAV rh8, AAV9, AAVrh10, and/or AAV11. In some aspects, the AAV comprises at least one AAV-DJ ITR and capsid protein from any of AAV1, AAV2, AAV3, AAV4, AAV5, AAV6, AAV7, AAV8, AAV rh8, AAV9, AAVrh10, and/or AAV11.

In some aspects, the viral particles comprise a recombinant self-complementing genome. AAV viral particles with self-complementing genomes and methods of use of self-complementing AAV genomes are described in U.S. Pat. Nos. 6,596,535; 7,125,717; 7,765,583; 7,785,888; 7,790,154; 7,846,729; 8,093,054; and 8,361,457; and Wang Z., et al., (2003) Gene Ther 10:2105-2111, each of which are incorporated herein by reference in its entirety. A rAAV comprising a self-complementing genome will quickly form a double stranded DNA molecule by virtue of its partially complementing sequences (e.g., complementing coding and non-coding strands). In some aspects, an AAV viral particle comprises an AAV genome, wherein the rAAV genome comprises a first heterologous polynucleotide sequence (e.g., a coding strand) and a second heterologous polynucleotide sequence (e.g., the noncoding or antisense strand) wherein the first heterologous polynucleotide sequence can form intrastrand base pairs with the second polynucleotide sequence along most or all of its length. In some aspects, the first heterologous polynucleotide sequence and a second heterologous polynucleotide sequence are linked by a sequence that facilitates intrastrand base-pairing; e.g., a hairpin DNA structure. Hairpin structures include, for example in siRNA molecules. In some aspects, the first heterologous polynucleotide sequence and a second heterologous polynucleotide sequence are linked by a mutated ITR (e.g., the right ITR). The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

Methods for production of rAAV vectors, including transfection, stable cell line production, and infectious hybrid virus production systems which include adenovirus-AAV hybrids, herpesvirus-AAV hybrids (Conway, J E et al., (1997) J. Virology 71(11):8780-8789) and baculovirus-AAV hybrids can be employed. Typically, rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a nucleic acid to be delivered (such as any of the DNA-targeting systems, fusion proteins, compositions or components thereof) flanked by at least one AAV ITR sequences; and 5) suitable media and media components to support rAAV production. In some aspects, the AAV rep and cap gene products may be from any AAV serotype. In general, but not obligatory, the AAV rep gene product is of the same serotype as the ITRs of the rAAV vector genome as long as the rep gene products may function to replicated and package the rAAV genome. Suitable media may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551. In some aspects, the AAV helper functions are provided by adenovirus or HSV. In some aspects, the AAV helper functions are provided by baculovirus and the host cell is an insect cell (e.g., Spodoptera frugiperda (Sf9) cells).

Suitable rAAV production culture media of the present invention may be supplemented with serum or serum-derived recombinant proteins at a level of 0.5%-20% (v/v or w/v). Alternatively, rAAV vectors may be produced in serum-free conditions which may also be referred to as media with no animal-derived products. Commercial or custom media designed to support production of rAAV vectors may also be supplemented with one or more cell culture components, including without limitation glucose, vitamins, amino acids, and or growth factors, in order to increase the titer of rAAV in production cultures.

rAAV production cultures can be grown under a variety of conditions (over a wide temperature range, for varying lengths of time, and the like) suitable to the particular host cell being utilized. rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

rAAV vector particles of the invention may be harvested from rAAV production cultures by lysis of the host cells of the production culture or by harvest of the spent media from the production culture, provided the cells are cultured under conditions to cause release of rAAV particles into the media from intact cells, as described in U.S. Pat. No. 6,566,118). Suitable methods of lysing cells include for example multiple freeze/thaw cycles, sonication, microfluidization, and treatment with chemicals, such as detergents and/or proteases.

In some aspects, recombinant viral particles for delivery of the nucleic acids, compositions or components thereof are highly purified, suitably buffered, and concentrated. In some aspects, the viral particles are concentrated to at least about 1×10⁷ vg/mL to about 9×10¹³ vg/mL or any concentration therebetween.

In some aspects, adeno-associated virus (AAV)-based vectors are generally used vector system for neurologic gene therapy, with an excellent safety record established in multiple clinical trials (Kaplitt et al., (2007) Lancet 369:2097-2105; Eberling et al., (2008) Neurology 70:1980-1983; Fiandaca et al., (2009) Neuroimage 47 Suppl. 2:T27-35). In some cases, effective treatment of neurologic disorders has been hindered by problems associated with the delivery of AAV vectors to affected cell populations. This delivery issue has been especially problematic for disorders involving the cerebral cortex. Simple injections do not distribute AAV vectors effectively, relying on diffusion, which is effective only within a 1- to 3-mm radius. An alternative method, convection-enhanced delivery (CED) (Nguyen et al., (2003) J. Neurosurg. 98:584-590), has been used clinically in gene therapy (AAV2-hAADC) for Parkinson's disease (Fiandaca et al., (2008) Exp. Neurol. 209:51-57). The underlying principle of CED involves pumping infusate into brain parenchyma under sufficient pressure to overcome the hydrostatic pressure of interstitial fluid, thereby forcing the infused particles into close contact with the dense perivasculature of the brain. Pulsation of these vessels acts as a pump, distributing the particles over large distances throughout the parenchyma (Hadaczek et al., (2006) Hum. Gene Ther. 17:291-302). To increase the safety and efficacy of CED a reflux-resistant cannula (Krauze et al., (2009) Methods Enzymol. 465:349-362) can be employed along with monitored delivery with real-time MRI. Monitored delivery allows for the quantification and control of aberrant events, such as cannula reflux and leakage of infusate into ventricles (Eberling et al., (2008) Neurology 70:1980-1983; Fiandaca et al., (2009) Neuroimage 47 Suppl. 2:T27-35; Saito et al., (2011) Journal of Neurosurgery Pediatrics 7:522-526).

In some aspects, the nucleic acid to be delivered is operably linked to a promoter. In some aspects, the promoter expresses the nucleic acid to be delivered in a cell of the CNS. In some aspects, the promoter expresses the nucleic acid to be delivered in a brain cell. In some aspects, the promoter expresses the nucleic acid to be delivered in a neuron and/or a glial cell. In some aspects, the neuron is a medium spiny neuron of the caudate nucleus, a medium spiny neuron of the putamen, a neuron of the cortex layer IV and/or a neuron of the cortex layer V. In some aspects, the glial cell is an astrocyte. In some aspects, the promoter is a CBA promoter, a minimum CBA promoter, a CMV promoter or a GUSB promoter. In some aspects, the promoter is inducible. In further embodiments, the rAAV vector comprises one or more of an enhancer, a splice donor/splice acceptor pair, a matrix attachment site, or a polyadenylation signal.

In some aspects, the methods for delivering a recombinant adeno-associated viral (rAAV) particle to the central nervous system of a subject involve administering the rAAV particle to the striatum, wherein the rAAV particle comprises a rAAV vector encoding a nucleic acid to be delivered that is expressed in at least the cerebral cortex and striatum of the subject. In some aspects, methods for delivering a rAAV particle to the central nervous system of a subject involve administering the rAAV particle to the striatum, wherein the rAAV particle comprises an rAAV vector encoding a nucleic acid to be delivered that is expressed in at least the cerebral cortex and striatum of the subject and wherein the rAAV particle comprises an AAV serotype 1 (AAV1) capsid. In some aspects, methods for delivering a rAAV particle to the central nervous system of a subject comprise administering the rAAV particle to the striatum, wherein the rAAV particle comprises an rAAV vector encoding a nucleic acid to be delivered that is expressed in at least the cerebral cortex and striatum of the subject and wherein the rAAV particle comprises an AAV serotype 2 (AAV2) capsid. In some aspects, methods for treating a central nervous system-related disease in a subject involve administering a rAAV particle to the striatum, wherein the rAAV particle comprises a rAAV vector encoding a nucleic acid to be delivered that is expressed in at least the cerebral cortex and striatum of the subject. In some aspects, the subject is a human.

In some aspects, a rAAV particle is administered to one or more regions of the central nervous system (CNS). In some aspects, the rAAV particle is administered to the striatum. The striatum is known as a region of the brain that receives inputs from the cerebral cortex (the term “cortex” may be used interchangeably herein) and sends outputs to the basal ganglia (the striatum is also referred to as the striate nucleus and the neostriatum). In some aspects, the striatum controls both motor movements and emotional control/motivation and has been implicated in many neurological diseases, such as Huntington's disease. Several cell types of interest are located in the striatum, including without limitation spiny projection neurons (also known as medium spiny neurons), GABAergic interneurons, and cholinergic interneurons. Medium spiny neurons make up most of the striatal neurons. These neurons are GABAergic and express dopamine receptors. Each hemisphere of the brain contains a striatum.

In some aspects, important substructures of the striatum include the caudate nucleus and the putamen. In some aspects, the rAAV particle is administered to the caudate nucleus (the term “caudate” may be used interchangeably herein). The caudate nucleus is known as a structure of the dorsal striatum. The caudate nucleus has been implicated in control of functions such as directed movements, spatial working memory, memory, goal-directed actions, emotion, sleep, language, and learning. Each hemisphere of the brain contains a caudate nucleus.

In some aspects, the rAAV particle is administered to the putamen. Along with the caudate nucleus, the putamen is known as a structure of the dorsal striatum. The putamen comprises part of the lenticular nucleus and connects the cerebral cortex with the substantia nigra and the globus pallidus. Highly integrated with many other structures of the brain, the putamen has been implicated in control of functions such as learning, motor learning, motor performance, motor tasks, and limb movements. Each hemisphere of the brain contains a putamen.

In some aspects, rAAV particles may be administered to one or more sites of the striatum. In some aspects, the rAAV particle is administered to the putamen and the caudate nucleus of the striatum. In some aspects, the rAAV particle is administered to the putamen and the caudate nucleus of each hemisphere of the striatum. In some aspects, the rAAV particle is administered to at least one site in the caudate nucleus and two sites in the putamen.

In some aspects, the rAAV particle is administered to one hemisphere of the brain. In some aspects, the rAAV particle is administered to both hemispheres of the brain. For example, in some aspects, the rAAV particle is administered to the putamen and the caudate nucleus of each hemisphere of the striatum. In some aspects, the composition containing rAAV particles is administered to the striatum of each hemisphere. In some aspects, the composition containing rAAV particles is administered to striatum of the left hemisphere or the striatum of the right hemisphere and/or the putamen of the left hemisphere or the putamen of the right hemisphere. In some aspects, the composition containing rAAV particles is administered to any combination of the caudate nucleus of the left hemisphere, the caudate nucleus of the right hemisphere, the putamen of the left hemisphere and the putamen of the right hemisphere.

In some aspects, the methods involving administration to CNS an effective amount of recombinant viral particles to the striatum can be employed for delivery, wherein the rAAV particle comprises a rAAV vector encoding a nucleic acid to be delivered that is expressed in at least the cerebral cortex and striatum. In some aspects, the viral titer of the rAAV particles is at least about any of 5×10¹², 6×10¹², 7×10¹², 8×10¹², 9×10¹², 10×10¹², 11×10¹², 15×10¹², 20×10¹², 25×10¹², 30×10¹², or 50×10¹² genome copies/mL. In some aspects, the viral titer of the rAAV particles is about any of 5×10¹² to 6×10¹², 6×10¹² to 7×10¹², 7×10¹² to 8×10¹², 8×10¹² to 9×10¹²,9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30×10¹² to 50×10¹², or 50×10¹² to 100×10¹² genome copies/mL. In some aspects, the viral titer of the rAAV particles is about any of 5×10¹² to 10×10¹², 10×10¹² to 25×10¹², or 25×10¹² to 50×10¹² genome copies/mL. In some aspects, the viral titer of the rAAV particles is at least about any of 5×10⁹, 6×10⁹, 7×10⁹, 8×10⁹, 9×10⁹, 10×10⁹, 11×10⁹, 15×10⁹, 20×10⁹, 25×10⁹, 30×10⁹, or 50×10⁹ transducing units/mL. In some aspects, the viral titer of the rAAV particles is about any of 5×10⁹ to 6×10⁹, 6×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹ or 50×10⁹ to 100×10⁹ transducing units/mL. In some aspects, the viral titer of the rAAV particles is about any of 5×10⁹ to 10×10⁹, 10×10⁹ to 15×10⁹, 15×10⁹ to 25×10⁹, or 25×10⁹ to 50×10⁹ transducing units/mL. In some aspects, the viral titer of the rAAV particles is at least any of about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰, 40×10¹⁰, or 50×10¹⁰ infectious units/mL. In some aspects, the viral titer of the rAAV particles is at least any of about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 11×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 40×10¹⁰, 40×10¹⁰ to 50×10¹⁰, or 50×10¹⁰ to 100×10¹⁰ infectious units/mL. In some aspects, the viral titer of the rAAV particles is at least any of about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL.

In some aspects, an effective amount of recombinant viral particles is administered to the striatum, wherein the rAAV particle comprises a rAAV vector encoding a nucleic acid to be delivered that is expressed in at least the cerebral cortex and striatum. In some aspects, the dose of viral particles administered to the individual is at least about any of 1×10⁸ to about 1×10¹³ genome copies/kg of body weight. In some aspects, the dose of viral particles administered to the individual is about 1×10⁸ to 1×10¹³ genome copies/kg of body weight.

In some aspects, an effective amount of recombinant viral particles is administered to the striatum, wherein the rAAV particle comprises a rAAV vector encoding a nucleic acid to be delivered that is expressed in at least the cerebral cortex and striatum. In some aspects, the total amount of viral particles administered to the individual is at least about 1×10⁹ to about 1×10¹⁴ genome copies. In some aspects, the total amount of viral particles administered to the individual is about 1×10⁹ to about 1×10¹⁴ genome copies.

In some embodiments, the vector is a non-viral vector. In some aspects, exemplary non-viral vectors include polymers, lipids, peptides, inorganic materials, and hybrid systems. In some aspects, the non-viral vector is a lipid nanoparticle (LNP), a liposome, an exosome, or a cell penetrating peptide. In some aspects, the non-viral vector is a lipid nanoparticle (LNP). In some aspects, the LNP can be used for delivery to the liver. Exemplary non-viral vectors include those described in WO 2020/051561, US 20210301274, Zu et al., The AAPS Journal volume 23, Article number: 78 (2021), and Sung et al., Biomaterials Research volume 23, Article number: 8 (2019), Nyamay'Antu et al., Cell & Gene Therapy Insights 2019; 5(S1):51-57, and Yin et al., Nature Reviews Genetics 15:541-555 (2014).

In some embodiments, the vector is a non-viral vector selected from: a lipid nanoparticle, a liposome, an exosome, or a cell penetrating peptide

In some embodiments, a vector described herein is or comprises a lipid nanoparticle (LNP). In some embodiments, any of the epigenetic-modifying DNA-targeting systems, gRNAs, Cas-gRNA combinations, polynucleotides, fusion proteins, or components thereof described herein, are incorporated in lipid nanoparticles (LNPs), such as for delivery. In some embodiments, the lipid nanoparticle is a vector for delivery. In some embodiments, the nanoparticle may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC 3-DMA, DLin-KC2-DMA and DODMA.

Lipid nanoparticles can be used for the delivery of encapsulated or associated (e.g., complexed) therapeutic agents, including nucleic acids and proteins, such as those encoding and/or comprising CRISPR/Cas systems. See, e.g., U.S. Pat. Nos. 10,723,692, 10,941,395, and WO 2015/035136.

In some embodiments, the provided methods involve use of a lipid nanoparticle (LNP) comprising mRNA, such as mRNA encoding a protein component of any of the provided DNA-targeting systems, for example any of the fusion proteins provided herein. In some embodiments, the mRNA can be produced using methods known in the art such as in vitro transcription. In some embodiments of the method, the mRNA comprises a 5′ cap. In some embodiments, the 5′ cap is an altered nucleotide on the 5′ end of primary transcripts such as messenger RNA. In some aspects, the 5′ caps of the mRNA improves one or more of RNA stability and processing, mRNA metabolism, the processing and maturation of an RNA transcript in the nucleus, transport of mRNA from the nucleus to the cytoplasm, mRNA stability, and efficient translation of mRNA to protein. In some embodiments, a 5′ cap can be a naturally-occurring 5′ cap or one that differs from a naturally-occurring cap of an mRNA. A 5′ cap may be any 5′ cap known to a skilled artisan. In certain embodiments, the 5′ cap is selected from the group consisting of an Anti-Reverse Cap Analog (ARCA) cap, a 7-methyl-guanosine (7mG) cap, a CleanCap® analog, a vaccinia cap, and analogs thereof. For instance, the 5′ cap may include, without limitation, an anti-reverse cap analogs (ARCA) (U.S. Pat. No. 7,074,596), 7-methyl-guanosine, CleanCap® analogs, such as Cap 1 analogs (Trilink; San Diego, CA), or enzymatically capped using, for example, a vaccinia capping enzyme or the like. In some embodiments, the mRNA may be polyadenylated. The mRNA may contain various 5′ and 3′ untranslated sequence elements to enhance expression of the encoded protein and/or stability of the mRNA itself. Such elements can include, for example, posttranslational regulatory elements such as a woodchuck hepatitis virus post-transcriptional regulatory element (WPRE). In some embodiments, the mRNA comprises at least one nucleoside modification. The mRNA may contain modifications of naturally-occurring nucleosides to nucleoside analogs. Any nucleoside analogs known in the art are envisioned. Such nucleoside analogs can include, for example, those described in U.S. Pat. No. 8,278,036. In certain embodiments of the method, the nucleoside modification is selected from the group consisting of a modification from uridine to pseudouridine and uridine to Nl-methyl pseudouridine. In particular embodiments of the method the nucleoside modification is from uridine to pseudouridine.

In some embodiments, LNPs useful for in the present methods comprise a cationic lipid selected from DLin-DMA (1,2-dilinoleyloxy-3-dimethylaminopropane), DLin-MC3-DM A (dilinoleylmethyl-4-dimethylaminobutyrate), DLin-KC2-DMA (2,2-dilinoleyl-4-(2-dimethylaminoethyl)-[1,3]-dioxolane), DODMA (1,2-dioleyloxy-N,N-dimethyl-3-aminopropane), SS-OP (Bis[2-(4-{2-[4-(cis-9 octadecenoyloxy)phenylacetoxy]ethyl}piperidinyl)ethyl]disulfide), and derivatives thereof. DLin-MC3-DMA and derivatives thereof are described, for example, in WO 2010/144740. DODMA and derivatives thereof are described, for example, in U.S. Pat. No. 7,745,651 and Mok et al. (1999), Biochimica et Biophysica Acta, 1419(2): 137-150. DLin-DMA and derivatives thereof are described, for example, in U.S. Pat. No. 7,799,565. DLin-KC2-DMA and derivatives thereof are described, for example, in U.S. Pat. No. 9,139,554. SS-OP (NOF America Corporation, White Plains, NY) is described, for example, at https://www.nofamerica.com/store/index.php?dispatch=products.view&product_id=962. Additional and non-limiting examples of cationic lipids include methylpyridiyl-dialkyl acid (MPDACA), palmitoyl-oleoyl-nor-arginine (PONA), guanidino-dialkyl acid (GUADACA), 1,2-di-O-octadecenyl-3-trimethylammonium propane (DOTMA), 1,2-dioleoyl-3-trimethylammonium-propane (DOTAP), Bis{2-[N-methyl-N-(a-D-tocopherolhemisuccinatepropyl)amino]ethyl}disulfide (SS-33/3AP05), Bis{2-[4-(a-D-tocopherolhemisuccinateethyl)piperidyl]ethyl}disulfide (SS33/4PE15), Bis{2-[4-(cis-9-octadecenoateethyl)-1-piperidinyl]ethyl}disulfide (SS18/4PE16), and Bis{2-[4-(cis,cis-9,12-octadecadienoateethyl)-1-piperidinyl]ethyl}disulfide (SS18/4PE13). In further embodiments, the lipid nanoparticles also comprise one or more non-cationic lipids and a lipid conjugate.

In some embodiments, the molar concentration of the cationic lipid is from about 20% to about 80%, from about 30% to about 70%, from about 40% to about 60%, from about 45% to about 55%, or about 20%, about 25%, about 30%, about 35%, about 40%, about 45%, about 50%, about 55%, about 60%, about 65%, about 70%, about 75%, or about 80% of the total lipid molar concentration, wherein the total lipid molar concentration is the sum of the cationic lipid, the non-cationic lipid, and the lipid conjugate molar concentrations. In certain embodiments, the lipid nanoparticles comprise a molar ratio of cationic lipid to any of the polynucleotides of from about 1 to about 20, from about 2 to about 16, from about 4 to about 12, from about 6 to about 10, or about 1, about 2, about 3, about 4, about 5, about 6, about 7, about 8, about 9, about 10, about 11, about 12, about 13, about 14, about 15, about 16, about 17, about 18, about 19, or about 20.

In some embodiments, the lipid nanoparticles can comprise at least one non-cationic lipid. In particular embodiments, the molar concentration of the non-cationic lipids is from about 20% to about 80%, from about 30% to about 70%, from about 40% to about 70%, from about 40% to about 60%, from about 46% to about 50%, or about 20%, about 25%, about 30%, about 35%, about 40%, about 45%, about 48.5%, about 50%, about 55%, about 60%, about 65%, about 70%, about 75%, or about 80% of the total lipid molar concentration. Non-cationic lipids include, in some embodiments, phospholipids and steroids.

In some embodiments, phospholipids useful for the lipid nanoparticles described herein include, but are not limited to, 1,2-Distearoyl-sn-glycero-3-phosphocholine (DSPC), 1,2-Didecanoyl-sn-glycero-3-phosphocholine (DDPC), 1,2-Dierucoyl-sn-glycero-3-phosphate(Sodium Salt) (DEPA-NA), 1,2-Dierucoyl-sn-glycero-3-phosphocholine (DEPC), 1,2-Dierucoyl-sn-glycero-3-phosphoethanolamine (DEPE), 1,2-Dierucoyl-sn-glycero-3[Phospho-rac-(1-glycerol)(Sodium Salt) (DEPG-NA), 1,2-Dilinoleoyl-sn-glycero-3-phosphocholine (DLOPC), 1,2-Dilauroyl-sn-glycero-3-phosphate(Sodium Salt) (DLPA-NA), 1,2-Dilauroyl-sn-glycero-3-phosphocholine (DLPC), 1,2-Dilauroyl-sn-glycero-3-phosphoethanolamine (DLPE), 1,2-Dilauroyl-sn-glycero-3[Phospho-rac-(1-glycerol.)(Sodium Salt) (DLPG-NA), 1,2-Dilauroyl-sn-glycero-3[Phospho-rac-(1-glycerol)(Ammonium Salt) (DLPG-NH4), 1,2-Dilauroyl-sn-glycero-3-phosphoserine(Sodium Salt) (DLPS-NA), 1,2-Dimyristoyl-sn-glycero-3-phosphate(SodiumSalt) (DMPA-NA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3[Phospho-rac-(1-glycerol)(Sodium Salt) (DMPG-NA), 1,2-Dimyristoyl-sn-glycero-3[Phospho-rac-(1-glycerol)(Ammonium Salt) (DMPG-NH4), 1,2-Dimyristoyl-sn-glycero-3[Phospho-rac-(1-glycerol)(Sodium/Ammonium Salt) (DMPG-NH4/NA), 1,2-Dimyristoyl-sn-glycero-3-phosphoserine(Sodium Salt) (DMPS-NA), 1,2-Dioleoyl-sn-glycero-3-phosphate(Sodium Salt) (DOPA-NA), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3[Phospho-rac-(1-glycerol)(Sodium Salt) (DOPG-NA), 1,2-Dioleoyl-sn-glycero-3-phosphoserine(Sodium Salt) (DOPS-NA), 1,2-Dipalmitoyl-sn-glycero-3-phosphate(Sodium Salt) (DPPA-NA), 1,2-Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero-3[Phospho-rac-(1-glycerol)(Sodium Salt) (DPPG-NA), 1,2-Dipalmitoyl-sn-glycero-3[Phospho-rac-(1-glycerol)(Ammonium Salt) (DPPG-NH4), 1,2-Dipalmitoyl-sn-glycero-3-phosphoserine(Sodium Salt) (DPPS-NA), 1,2-Distearoyl-sn-glycero-3-phosphate(Sodium Salt) (DSPA-NA), 1,2-Distearoyl-sn-glycero-3-phosphoethanolamine (DSPE), 1,2-Distearoyl-sn-glycero-3[Phospho-rac-(1-glycerol)(Sodium Salt) (DSPG-NA), 1,2-Distearoyl-sn-glycero-3[Phospho-rac-(1-glycerol)(Ammonium Salt) (DSPG-NH4), 1,2-Distearoyl-sn-glycero-3-phosphoserine(Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LY S OPCM YRIS TIC), 1-Palmitoyl-sn-glycero-3-phosphocholine (LYSOPCPALMITIC), 1-Stearoyl-sn-glycero-3-phosphocholine (LYSOPC STEARIC), 1-Myristoyl-2-palmitoyl-sn-glycero3-phosphocholine (MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3[Phospho-rac-(1-glycerol)](Sodium Salt) (POPG-NA), 1-Palmitoyl-2-stearoyl-sn-glycero-3-phosphocholine (PS PC), 1-Stearoyl-2-myristoyl-sn-glycero-3-phosphocholine (SMPC), 1-Stearoyl-2-oleoyl-sn-glycero-3-phosphocholine (SOPC), and 1-Stearoyl-2-palmitoyl-sn-glycero-3-phosphocholine (SPPC). In particular embodiments, the phospholipid is DSPC. In particular embodiments, the phospholipid is DOPE. In particular embodiments, the phospholipid is DOPC.

In some embodiments, the non-cationic lipids comprised by the lipid nanoparticles include one or more steroids. Steroids useful for the lipid nanoparticles described herein include, but are not limited to, cholestanes such as cholesterol, cholanes such as cholic acid, pregnanes such as progesterone, androstanes such as testosterone, and estranes such as estradiol. Further steroids include, but are not limited to, cholesterol (ovine), cholesterol sulfate, desmosterol-d6, cholesterol-d7, lathosterol-d7, desmosterol, stigmasterol, lanosterol, dehydrocholesterol, dihydrolanosterol, zymosterol, lathosterol, zymosterol-d5, 14-demethyl-lanosterol, 14-demethyl-lanosterol-d6, 8(9)-dehydrocholesterol, 8(14)-dehydrocholesterol, diosgenin, DHEA sulfate, DHEA, lanosterol-d6, dihydrolanosterol-d7, campesterol-d6, sitosterol, lanosterol-95, Dihydro FF-MAS-d6, zymostenol-d7, zymostenol, sitostanol, campestanol, campesterol, 7 -dehydrodesmosterol, pregnenolone, sitosterol-d7, Dihydro T-MAS, Delta 5-avenasterol, Brassicasterol, Dihydro FF-MAS, 24-methylene cholesterol, cholic acid derivatives, cholesteryl esters, and glycosylated sterols. In particular embodiments, the lipid nanoparticles comprise cholesterol.

In some embodiments, the lipid nanoparticles comprise a lipid conjugate. Such lipid conjugates include, but are not limited to, ceramide PEG derivatives such as C8 PEG2000 ceramide, C16 PEG2000 ceramide, C8 PEG5000 ceramide, C16 PEG5000 ceramide, C8 PEG750 ceramide, and C16 PEG750 ceramide, phosphoethanolamine PEG derivatives such as 16:0 PEG5000PE, 14:0 PEG5000 PE, 18:0 PEG5000 PE, 18:1 PEG5000 PE, 16:0 PEG3000 PE, 14:0 PEG3000 PE, 18:0 PEG3000 PE, 18:1 PEG3000 PE, 16:0 PEG2000 PE, 14:0 PEG2000 PE, 18:0 PEG2000 PE, 18:1 PEG2000 PE 16:0 PEG1000 PE, 14:0 PEG1000 PE, 18:0 PEG1000 PE, 18:1 PEG 1000 PE, 16:0 PEG750 PE, 14:0 PEG750 PE, 18:0 PEG750 PE, 18:1 PEG750 PE, 16:0 PEG550 PE, 14:0 PEG550 PE, 18:0 PEG550 PE, 18:1 PEG550 PE, 16:0 PEG350 PE, 14:0 PEG350 PE, 18:0 PEG350 PE, and 18:1 PEG350, sterol PEG derivatives such as Chol-PEG600, and glycerol PEG derivatives such as DMG-PEG5000, DSG-PEG5000, DPG-PEG5000, DMG-PEG3000, DSG-PEG3000, DPG-PEG3000, DMG-PEG2000, DSG-PEG2000, DPG-PEG2000, DMG-PEG1000, DSG-PEG1000, DPG-PEG1000, DMG-PEG750, DSG-PEG750, DPG-PEG750, DMG-PEG550, DSG-PEG550, DPG-PEG550, DMG-PEG350, DSG-PEG350, and DPG-PEG350. In some embodiments, the lipid conjugate is a DMG-PEG. In some particular embodiments, the lipid conjugate is DMG-PEG2000. In some particular embodiments, the lipid conjugate is DMG-PEG5000.

It is within the level of a skilled artisan to select the cationic lipids, non-cationic lipids and/or lipid conjugates which comprise the lipid nanoparticle, as well as the relative molar ratio of such lipids to each other, such as based upon the characteristics of the selected lipid(s), the nature of the delivery to the intended target cells, and the characteristics of the nucleic acids and/or proteins to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method can be prepared by various techniques which are known to a skilled artisan. Nucleic acid-lipid particles and methods of preparation are disclosed in, for example, U.S. Patent Publication Nos. 20040142025 and 20070042031.

In some embodiments, the lipid nanoparticles will have a size within the range of about 25 to about 500 nm. In some embodiments, the lipid nanoparticles have a size from about 50 nm to about 300 nm, or from about 60 nm to about 120 nm. The size of the lipid nanoparticles may be determined by quasi-electric light scattering (QELS) as described in Bloomfield, Ann. Rev. Biophys. Bioeng., 10:421A150 (1981). A variety of methods are known in the art for producing a population of lipid nanoparticles of particular size ranges, for example, sonication or homogenization. One such method is described in U.S. Pat. No. 4,737,323.

In some embodiments, the lipid nanoparticles comprise a cell targeting molecule such as, for example, a targeting ligand (e.g., antibodies, scFv proteins, DART molecules, peptides, aptamers, and the like) anchored on the surface of the lipid nanoparticle that selectively binds the lipid nanoparticles to the targeted cell, such as any cell described herein.

In some embodiments, the vector exhibits tropism for one or more cell types. For example, the vector may exhibit liver cell and/or hepatocyte tropism, neural cell (e.g. neuron or glia) tropism, immune cell tropism, or tropism for any suitable cell type.

In some aspects, provided herein are pluralities of vectors that comprise any of the vectors described herein, and one or more additional vectors. In some embodiments, the one or more additional vectors comprise one or more additional polynucleotides encoding any additional transcriptional activation domain, multipartite effector such as multipartite activator, DNA-targeting domain, gRNA, fusion protein, DNA-targeting system, or a portion, component, or combination thereof. In some aspects, provided are pluralities of vectors, that include: a first vector comprising any of the polynucleotides described herein; a second vector comprising any of the polynucleotides described herein; and optionally one or more additional vectors comprising any of the polynucleotides described herein.

In some aspects, vectors provided herein may be referred to as delivery vehicles. In some aspects, any of the DNA-targeting systems, components thereof, or polynucleotides disclosed herein can be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Methods of introducing a nucleic acid into a host cell are known in the art, and any known method can be used to introduce a nucleic acid (e.g., an expression construct) into a cell. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. Direct delivery of the RNP complex, including the DNA-targeting domain complexed with the sgRNA, can eliminate the need for intracellular transcription and translation and can offer a robust platform for host cells with low transcriptional and translational activity. The RNP complexes can be introduced into the host cell by any of the methods known in the art.

Nucleic acids or RNPs of the disclosure can be incorporated into a host using virus-like particles (VLP). VLPs contain normal viral vector components, such as envelope and capsids, but lack the viral genome. For instance, nucleic acids expressing the Cas and sgRNA can be fused to the viral vector components such as gag and introduced into producer cells. The resulting virus-like particles containing the sgRNA-expressing vectors can infect the host cell for efficient editing.

Introduction of the complexes, polypeptides, and nucleic acids of the disclosure can occur by protein transduction domains (PTDs). PTDs, including the human immunodeficiency virus-1 TAT, herpes simplex virus-1 VP22, Drosophila Antennapedia Antp, and the polyarginines, are peptide sequences that can cross the cell membrane, enter a host cell, and deliver the complexes, polypeptides, and nucleic acids into the cell.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like, for example as described in WO 2017/193107, WO 2016/123578, WO 2014/152432, WO 2014/093661, WO 2014/093655, or WO 2021/226555.

Various methods for the introduction of polynucleotides are well known and may be used with the provided methods and compositions. Exemplary methods include those for transfer of polynucleotides encoding the DNA targeting systems provided herein, including via viral, e.g., retroviral or lentiviral, transduction, transposons, and electroporation.

### VII. Pharmaceutical Compositions and Formulations

In some aspects, provided herein are compositions, such as pharmaceutical compositions and formulations for administration, that include any of the transcriptional activation domains, multipartite effector such as multipartite activators, DNA-targeting domains, gRNAs, fusion proteins, or DNA-targeting systems described herein, or a portion, component, or combination of any of the foregoing, or any of the polynucleotides or vectors encoding the same. In some aspects, the pharmaceutical composition contains one or more DNA-targeting systems provided herein or a component thereof. In some aspects, the pharmaceutical composition comprises one or more vectors that contain polynucleotides that encode one or more components of the DNA-targeting systems provided herein. Such compositions can be used in accord with the provided methods, and/or with the provided articles of manufacture or compositions, such as in the prevention or treatment of diseases, conditions, and disorders, or in detection, diagnostic, and prognostic methods. In some aspects, the pharmaceutical composition can be used in any of the methods and treatments as disclosed herein, and in the preparation of a medicament in order to carry out such therapeutic methods.

The term “pharmaceutical formulation” refers to a preparation which is in such form as to permit the biological activity of an active ingredient contained therein to be effective, and which contains no additional components which are unacceptably toxic to a subject or a cell to which the formulation would be administered.

In some embodiments, the pharmaceutical composition may further comprise a pharmaceutically acceptable excipient. The pharmaceutically acceptable excipient may be functional molecules as vehicles, adjuvants, carriers, or diluents.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

In some embodiments, the transfection facilitating agent is a polyanion, polycation, including poly-L-glutamate (LGS), or lipid. In some embodiments, the transfection facilitating agent is poly-L-glutamate. In some embodiments, the transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the DNA-targeting system may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent in a solvent, such as in admixture with a suitable carrier, diluent, or excipient such as sterile water, physiological saline, glucose, dextrose, or the like. The formulations to be used for in vivo or ex vivo administration or use are generally sterile. Sterility may be readily accomplished, e.g., by filtration through sterile filtration membranes.

The pharmaceutical composition in some embodiments contains components in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the composition can be administered to a subject by any suitable means, for example, by bolus infusion or by injection, e.g., by intravenous or subcutaneous injection. In some embodiments, a given dose is administered by a single bolus administration of the composition. In some embodiments, the composition is administered by multiple bolus administrations of the composition, for example, over a period of no more than 3 days, or by continuous infusion administration of the composition. In some embodiments, the composition is administered parenterally, for example by intravenous, intramuscular, subcutaneous, or intraperitoneal administration. In some embodiments, the composition is administered to a subject using peripheral systemic delivery by intravenous, intraperitoneal, or subcutaneous injection.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

### VIII. Methods of Transcriptional Activation

In some aspects, provided herein are methods for modulating (e.g. increasing transcription of) the expression of a gene in a cell. In some embodiments, provided herein are methods for targeted transcriptional activation, such as methods for specifically increasing the transcription of one or more target genes. In some embodiments, the method includes introducing into the cell any of the transcriptional activation domains, multipartite effector such as multipartite activators, DNA-targeting domains, gRNAs, fusion proteins, DNA-targeting systems, polynucleotides, or vectors described herein. In some embodiments, the method includes introducing into the cell any of the DNA-targeting systems described herein, or a polynucleotide or vector containing or encoding the same. In some embodiments, the DNA-targeting system recruits two or more transcriptional activation domains and/or a multipartite effector such as multipartite activator described herein to a target site for the target gene, thereby increasing transcription of the target gene. In some embodiments, the DNA-targeting system targets and increases transcription of one or more genes. In some embodiments, the introducing into the cell is performed using any suitable method or composition, such as any described in Section VI or VII.

In some embodiments, the transcription of the one or more genes, such as one or more target genes described herein, is increased in comparison to a comparable unmodified cell not subjected to the method, i.e. not contacted or introduced with the DNA-targeting system. In some embodiments, the transcription of the one or more genes is increased by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, 5-fold, 10-fold, 20-fold, 30-fold, 40-fold, 50-fold, 100-fold, 200-fold, 300-fold, 400-fold, 500-fold, 1000-fold or greater. In some embodiments, the transcription is stably increased or transiently increased. In some embodiments, the increased transcription of the one or more genes promotes a phenotype, such as any phenotype described herein. In some embodiments, the increased transcription is measured by any suitable method, including but limited to: real-time quantitative RT (reverse transcriptase)-polymerase chain reaction (qRT-PCR), Northern Blot, microarray analysis, or RNA sequencing (RNA-Seq).

### IX. Methods of Treatment

Provided herein are methods of treatment, e.g., including administering any of the compositions, such as pharmaceutical compositions described herein. In some aspects, also provided are methods of administering any of the compositions described herein to a subject, such as a subject that has a disease or disorder. The compositions, such as pharmaceutical compositions, described herein are useful in a variety of therapeutic, diagnostic and prophylactic indications. For example, the compositions are useful in treating a variety of diseases and disorders in a subject. Such methods and uses include therapeutic methods and uses, for example, involving administration of the compositions, to a subject having a disease, condition, or disorder. In some embodiments, the compositions are administered in an effective amount to effect treatment of the disease or disorder. Uses include uses of the compositions in such methods and treatments, and in the preparation of a medicament in order to carry out such therapeutic methods. In some embodiments, the methods are carried out by administering the compositions to the subject having or suspected of having the disease or condition. In some embodiments, the methods thereby treat the disease or condition or disorder in the subject. Also provided are therapeutic methods for administering the compositions to subjects, e.g., patients.

In some embodiments, the compositions include any of the transcriptional activation domains, multipartite effector such as multipartite activators, fusion proteins, or DNA-targeting systems provided herein, or a polynucleotide or vector encoding the same, in which delivery of the composition to a subject increases transcription of one or more genes in a cell in the subject. In some embodiments, the increased transcription modulates one or more activities or function of a cell in the subject. In some embodiments, the subject has or is suspected of having a condition, such as a disease or disorder. In some embodiments, the increased transcription treats the condition in the subject.

Targeted transcriptional activation with programmable DNA-targeting systems provides a tool for investigating the role of specific genes and their biological effects, such as on cellular phenotypes. CRISPR/Cas-based DNA-targeting systems in particular can facilitate high-throughput screens to identify genes that promote or repress a phenotype of interest. Targeted transcriptional activation can also be used for therapeutic applications, e.g. by promoting a desired phenotype in a cell or a subject. Certain diseases and conditions are caused or exacerbated by reduced transcription of a gene that is essential to normal cellular function, as exemplified by reduced expression of frataxin in Friedreich's ataxia. In such diseases, targeted transcriptional activation of the affected gene could provide a therapeutic benefit.

In some embodiments, the subject has or is suspected of having Friedreich's ataxia (FA), resulting from a mutation in a frataxin (FXN) gene that causes reduced FXN expression. In some embodiments, administration or use of a composition that includes a DNA-targeting system provided herein, or a polynucleotide or vector encoding the same, increases expression of FXN, thereby treating the disease.

In some embodiments, the subject has or is suspected of having a condition, such as a disease or disorder, for which increased transcription of a gene is therapeutic, such as any gene listed in Table 3. In some embodiments, administration or use of a composition that includes a DNA-targeting system provided herein, or a polynucleotide or vector encoding the same, increases expression of the gene, thereby treating the condition.

In some embodiments, the methods of administering a composition provided herein to a subject are carried out in vivo (i.e. in a subject).

In some embodiments, the methods of administering a composition provided herein to a subject a are carried out ex vivo, such as in a cell. In some embodiments, the cell is a cell from the subject, such as a primary cell. In some embodiments, the cell is a cell that is derived from the subject, such as a descendant of a primary cell, an induced pluripotent stem cell (iPSC), or an engineered and/or modified cell (such as a CAR T cell). In some embodiments, the method modifies the phenotype of the cell. In some embodiments, the cell is administered to the subject.

In some aspects, increased transcription in a cell or a modified phenotype of a cell or a subject resulting from the methods provided herein are measured by any of a number of known methods. In some aspects measured by assessing clinical outcome. Specific thresholds for the parameters can be set to determine the efficacy of the methods of therapy provided herein.

### X. Kits and Articles of Manufacture

Also provided are articles of manufacture, systems, apparatuses, and kits useful in performing the provided embodiments. In some embodiments, the provided articles of manufacture or kits contain any of the transcriptional activation domains, multipartite effectors such as multipartite activators, DNA-targeting domains, fusion proteins, DNA-targeting systems, polynucleotides, vectors, pharmaceutical compositions, cells, or a portion or a component of any of the foregoing, or any combination thereof. In some embodiments, the articles of manufacture or kits include polypeptides, polynucleotides, nucleic acids, vectors, and/or cells useful in performing the provided methods.

In some embodiments, the articles of manufacture or kits include one or more containers, typically a plurality of containers, packaging material, and a label or package insert on or associated with the container or containers and/or packaging, generally including instructions for use, e.g., instructions for introducing or administering.

Also provided are articles of manufacture, systems, apparatuses, and kits useful in administering the provided compositions, e.g., pharmaceutical compositions, e.g., for use in therapy or treatment. In some embodiments, the articles of manufacture or kits provided herein contain vectors and/or plurality of vectors, such as any vectors and/or plurality of vectors described herein. In some aspects, the articles of manufacture or kits provided herein can be used for administration of the vectors and/or plurality of vectors, and can include instructions for use.

The articles of manufacture and/or kits containing compositions for therapy, may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### XI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”. In some embodiments, “about” may refer to ±25%, ±20%, ±15%, +10%, ±5%, or ±1%.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, corresponding residues can be identified, for example, using conserved and identical amino acid residues as guides. In general, to identify corresponding positions, the sequences of amino acids are aligned so that the highest order match is obtained (see, e.g.: Computational Molecular Biology, Lesk, A.M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

A “gene,” includes a DNA region encoding a gene product. Thus, the gene typically refers to coding and/or transcribed sequences. The sequence of a gene is typically present at a fixed chromosomal position or locus on a chromosome in the cell.

A “regulatory element” or “DNA regulatory element,” which terms are used interchangeably herein, in reference to a gene refers to DNA regions which regulate the production of a gene product, whether or not such regulatory sequences are adjacent to coding and/or transcribed sequences. Accordingly, a regulatory element includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, a “target site” or “target nucleic acid sequence” is a nucleic acid sequence that defines a portion of a nucleic acid to which a binding molecule (e.g. a DNA-targeting domain disclosed herein) will bind, provided sufficient conditions for binding exist.

The term “expression” with reference to a gene or “gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or can be a protein produced by translation of an mRNA. For instance, expression includes the transcription and/or translation of a particular nucleotide sequence drive by its promoter. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Hence, reference to expression or gene expression includes protein (or polypeptide) expression or expression of a transcribable product of or a gene such as mRNA. The protein expression may include intracellular expression or surface expression of a protein. Typically, expression of a gene product, such as mRNA or protein, is at a level that is detectable in the cell.

In some aspects, a “detectable” expression level, means a level that is detectable by standard techniques known to a skilled artisan, and include for example, differential display, RT (reverse transcriptase)-coupled polymerase chain reaction (PCR), Northern Blot, and/or RNase protection analyses as well as immunoaffinity-based methods for protein detection, such as flow cytometry, ELISA, or western blot. The degree of expression levels need only be large enough to be visualized or measured via standard characterization techniques.

In some aspects, the term “transcription activation” or “transcriptional activation” can refer to starting or increasing transcription from a particular gene or locus, such as the production or generation of messenger RNA (mRNA) from a gene or a locus, such as a coding gene or locus, or an increase in production or generation of rnRNA. In some aspects, the term “transcription activation” or “transcriptional activation” also includes an increased or an enhanced mRNA expression from a gene or a locus.

In some aspects, the term “transcription activation domain” or “transcriptional activation domain” can refer to a polypeptide domain or fragment that is capable of activating, inducing, catalyzing or leading to transcription activation, transcription co-activation, transcription elongation, or transcription de-repression. In some aspects, the term “transcription activation domain” or “transcriptional activation domain” includes a portion, fragment, domain or variant of a protein or a polypeptide that exhibits transcriptional activation, is capable of inducing or activating transcription from a gene), is a functional transcriptional activation domain, and/or has a function of transcription activation, and/or that is capable of leading to increased transcription from a gene by at least 5%, 10%, 20%, 30%, 40% or 50%, 60%, 70%, 80%, 85%, 90%, or 100% or more, such as 2-fold, 5-fold, 10-fold, 20-fold, 30-fold, 40-fold, 50-fold, 60-fold, 70-fold, 80-fold, 90-fold, 100-fold, 200-fold, 300-fold, 400-food, 500-fold, 1000-fold or more, compared to the absence of the transcriptional activation domain.

In some aspects, the term “increased expression”, “enhanced expression” or “overexpression” means any form of expression that is additional to the expression in an original or source cell that does not contain the modification for modulating a particular gene expression by a DNA-targeting system, for instance a wild-type expression level (Which can be absence of expression or immeasurable expression as well). Reference herein to “increased expression,” “enhanced expression” or “overexpression” is taken to mean an increase in gene expression relative to the level in a cell that does not contain the modification, such as the original source cell prior to contacting with, or engineering to introduce, the DNA-targeting system or fusion protein into a cell. The increase in expression can be at least 5%, 10%, 20%, 30%, 40% or 50%, 60%, 70%, 80%, 85%, 90%, or 100% or more. In some cases, the increase in expression can be at least 2-fold, 5-fold, 10-fold, 20-fold, 30-fold, 40-fold, 50-fold, 60-fold, 70-fold, 80-fold, 90-fold, 100-fold, 200-fold, 300-fold, 400-food, 500-fold, 1000-fold or more.

In some aspects, the term “reduced expression” or “decreased expression” means any form of expression that is lower than the expression in an original or source cell that does not contain the modification for modulating a particular gene expression by a DNA-targeting system, for instance a wild-type expression level (which can be absence of expression or immeasurable expression as well). Reference herein to “reduced expression,” or “decreased expression” is taken to mean a decrease in gene expression relative to the level in a cell that does not contain the modification, such as the original source cell prior to contacting with, or engineering to introduce, the DNA-binding system into a cell. The decrease in expression can be at least 5%, 10%, 20%, 30%, 40% or 50%, 60%, 70%, 80%, 85%, 90%, or 100% or more. In some cases, the decrease in expression can be at least 2-fold, 5-fold, 10-fold, 20-fold, 30-fold, 40-fold, 50-fold, 60-fold, 70-fold, 80-fold, 90-fold, 100-fold, 200-fold or more.

In some aspects, the term “increased transcription” refers to the level of transcription of a gene that is additional to the transcription of the gene in an original or source cell that does not contain the modification for modulating transcription by a DNA-targeting system, for instance a wild-type transcription level of a gene. Reference to increased transcription can refer to an increase in the levels of a transcribable product of a gene such as mRNA. Any of a variety of methods can be used to monitor or quantitate a level of a transcribable product such as mRNA, including but not limited to, real-time quantitative RT (reverse transcriptase)-polymerase chain reaction (qRT-PCR), Northern Blot, microarray analysis, or RNA sequencing (RNA-Seq). The increase in transcription can be at least 5%, 10%, 20%, 30%, 40% or 50%, 60%, 70%, 80%, 85%, 90%, or 100% or more. In some cases, the increase in transcription can be at least 2-fold, 5-fold, 10-fold, 20-fold, 30-fold, 40-fold, 50-fold, 60-fold, 70-fold, 80-fold, 90-fold, 100-fold, 200-fold or more.

In some aspects, the term “reduced transcription” or “decreased transcription” refers to the level of transcription of a gene that is lower than the transcription of the gene in an original or source cell that does not contain the modification for modulating transcription by a DNA-targeting system, for instance a wild-type transcription level of a gene. Reference to reduced transcription or decreased transcription can refer to reduction in the levels of a transcribable product of a gene such as mRNA. Any of a variety of methods can be used to monitor or quantitate a level of a transcribable product such as mRNA, including but not limited to, real-time quantitative RT (reverse transcriptase)-polymerase chain reaction (qRT-PCR), Northern Blot, microarray analysis, or RNA sequencing (RNA-Seq). The increase in transcription can be at least 5%, 10%, 20%, 30%, 40% or 50%, 60%, 70%, 80%, 85%, 90%, or 100% or more. In some cases, the increase in transcription can be at least 2-fold, 5-fold, 10-fold, 20-fold, 30-fold, 40-fold, 50-fold, 60-fold, 70-fold, 80-fold, 90-fold, 100-fold, 200-fold or more.

In some aspects, an “epigenetic modification” refers to changes in the gene expression that are non-genetic modifications, i.e. not caused by changes in the DNA sequences, but are due to epigenetic changes such as events like DNA methylations or histone modifications. An epigenetic modification may result in a heritable change in gene activity and expression that occur without alteration in DNA sequence. For instance, epigenetic modifications include non-genetic modifications such as chemical modifications to the cytosine residues of DNA (DNA methylation) and histone proteins associated with DNA (histone modifications).

In some aspects, the term “modification” or “modified” with reference to a cell refers to any change or alteration in a cell that impacts gene expression in the cell. In some embodiments, the modification is an epigenetic modification that directly changes the epigenetic state of a gene or regulatory elements thereof to alter (e.g. reduce or increase) expression of a gene product. In some embodiments, a modification described herein results in reduced expression of a target gene or selected polynucleotide sequence.

In some aspects, a “fusion” molecule is a molecule in which two or more subunit molecules are linked, such as covalently. Examples of a fusion molecule include, but are not limited to, fusion proteins (for example, a fusion between a DNA-binding domain such as a ZFP, TALE DNA-binding domain or CRISPR-Cas protein and one or more effector domains, such as a transactivation domain). The fusion molecule also may be part of a system in which a polynucleotide component associates with a polypeptide component to form a functional molecule (e.g., a CRISPR/Cas system in which a single guide RNA associates with a functional domain to modulate gene expression). Fusion molecules also include fusion nucleic acids, for example, a nucleic acid encoding the fusion protein. Expression of a fusion protein in a cell can result from delivery of the fusion protein to the cell or by delivery of a polynucleotide encoding the fusion protein to a cell, where the polynucleotide is transcribed, and the transcript is translated, to generate the fusion protein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as adenoviral vectors or lentiviral vectors.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include, but are not limited to, cosmids, plasmids (e.g., naked or contained in liposomes) and viruses (e.g., lentiviruses, retroviruses, adenoviruses, and adeno-associated viruses) that incorporate the recombinant polynucleotide.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomelic “nucleotides.” The monomelic nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In some aspects, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. In some aspects, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

In some aspects, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various known ways, in some embodiments, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared.

In some embodiments, “operably linked” may include the association of components, such as a DNA sequence, (e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

In some aspects, a composition refers to any mixture of two or more products, substances, or compounds, including cells. It may be a solution, a suspension, liquid, powder, a paste, aqueous, non-aqueous or any combination thereof.

In some aspects, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human. In some embodiments, the subject is a patient that is known or suspected of having a disease, disorder or condition.

In some aspects, the term “treating” and “treatment” includes administering to a subject an effective amount of a biological molecule, such as a therapeutic agent, so that the subject has a reduction in at least one symptom of the disease or an improvement in the disease, for example, beneficial or desired clinical results. For instance, a biological molecule may include cells (e.g. liver cells), such as cells that have been modified by a DNA-targeting system or polynucleotide(s) encoding the DNA-targeting system described herein. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a disease or disorder are alleviated by at least 5%, at least 10%, at least 20%, at least 30%, at least 40%, or at least 50% upon treatment of the disease.

For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a biological molecule, such as a compound or cells, that, when administered, is sufficient to prevent development of, or alleviate to some extent, one or more of the signs or symptoms of the disorder or disease being treated. The therapeutically effective amount will vary depending on the biological molecule, the disease and its severity and the age, weight, etc., of the subject to be treated.

### XII. Exemplary Embodiments

Among the provided embodiments are:

1. A fusion protein comprising:
two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2, wherein the transcriptional activation domain increases transcription of an endogenous locus when recruited to a target site at the endogenous locus.

2. The fusion protein of embodiment 1, further comprising a DNA-targeting domain or a component thereof.

3. A fusion protein comprising:
(1) a DNA-targeting domain or a component thereof, and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2, wherein the transcriptional activation domain increases transcription of an endogenous locus when recruited to a target site at the endogenous locus.

4. The fusion protein of embodiment 3, wherein the DNA-targeting domain comprises a Cas-gRNA combination comprising a Cas protein or a variant thereof, and at least one gRNA that binds to the target site at the endogenous locus, and the component thereof fused to the two or more transcriptional activation domains is the Cas protein or a variant thereof.

5. The fusion protein of embodiment 3, wherein the DNA-targeting domain comprises a zinc finger protein (ZFP), a transcription activator-like effector (TALE), a meganuclease, a homing endonuclease, or an I-SceI enzyme or a variant thereof that binds to the target site at the endogenous locus.

6. A fusion protein comprising:
(1) a Cas protein or a variant thereof, and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2, wherein the transcriptional activation domain increases transcription of an endogenous locus when recruited to a target site at the endogenous locus.

7. The fusion protein of embodiment 4 or 6, wherein the Cas protein or a variant thereof is capable of complexing with at least one gRNA.

8. A fusion protein comprising:
(1) a zinc finger protein (ZFP), a transcription activator-like effector (TALE), a meganuclease, a homing endonuclease, or an I-SceI enzyme or a variant thereof, and (2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2, wherein the transcriptional activation domain increases transcription of an endogenous locus when recruited to a target site at the endogenous locus.

9. The fusion protein of any of embodiments 4-8, wherein the variant thereof comprises a catalytically inactive variant.

10. The fusion protein of any of embodiments 4, 6, and 7, wherein the Cas protein or a variant thereof is a Cas9 or a variant thereof.

11. The fusion protein of any of embodiments 4, 6, 7, and 10, wherein the Cas protein or a variant thereof protein is a deactivated Cas9 (dCas9).

12. The fusion protein of any of embodiments 4, 6, 7, 10 and 11, wherein the Cas protein or a variant thereof is a Staphylococcus aureus Cas9 (SaCas9) or a variant thereof.

13. The fusion protein of any of embodiments 4, 6, 7, and 10-12, wherein the Cas protein or a variant thereof is a Staphylococcus aureus dCas9 (dSaCas9) that comprises at least one amino acid mutation selected from D10A and N580A, with reference to numbering of positions of SEQ ID NO:3.

14. The fusion protein of any of embodiments 4, 6, 7, and 10-13, wherein the Cas protein or a variant thereof comprises the sequence set forth in SEQ ID NO:2, and an amino acid sequence that has at least 90%, 91%, 92%, 93%, 94%, 95%, 96%, 97%, 98%, and 99% sequence identity thereto.

15. The fusion protein of any of embodiments 4, 6, 7, 10 and 11, wherein the Cas9 or variant thereof is a Streptococcus pyogenes Cas9 (SpCas9) or a variant thereof.

16. The fusion protein of any of embodiments 4, 6, 7, 10, 11, and 15, wherein the Cas protein or a variant thereof is a Streptococcus pyogenes dCas9 (dSpCas9) that comprises at least one amino acid mutation selected from D10A and H840A, with reference to numbering of positions of SEQ ID NO:7.

17. The fusion protein of any of embodiments 4, 6, 7, 10, 11, 15, and 16, wherein the Cas protein or a variant thereof comprises the sequence set forth in SEQ ID NO:6, and an amino acid sequence that has at least 90%, 91%, 92%, 93%, 94%, 95%, 96%, 97%, 98%, and 99% sequence identity thereto.

18. The fusion protein of any of embodiments 4, 6, 7, and 10-17, wherein the Cas protein or a variant thereof is a split variant Cas protein, wherein the split variant Cas protein comprises a first polypeptide comprising an N-terminal fragment of the variant Cas protein and an N-terminal Intein, and a second polypeptide comprising a C-terminal fragment of the variant Cas protein and a C-terminal Intein.

19. The fusion protein of embodiment 4, 6, 7, and 10-18, wherein when the first polypeptide and the second polypeptide of the split variant Cas protein are present in proximity or present in the same cell, the N-terminal Intein and C-terminal Intein self-excise and ligate the N-terminal fragment and the C-terminal fragment of the variant Cas protein to form a full-length variant Cas protein.

20. The fusion protein of embodiment 18 or 19, wherein the N-terminal Intein comprises an N-terminal Npu Intein, or the sequence set forth in SEQ ID NO:88, or an amino acid sequence that has at least 90%, 91%, 92%, 93%, 94%, 95%, 96%, 97%, 98%, or 99% sequence identity thereto, or a portion of any of the foregoing.

21. The fusion protein of any of embodiments 18-20, wherein the N-terminal fragment of the variant Cas protein comprises:
the N-terminal fragment of variant SpCas9 from the N-terminal end up to position 573 of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or
the sequence set forth in SEQ ID NO:86, or an amino acid sequence that has at least 90%, 91%, 92%, 93%, 94%, 95%, 96%, 97%, 98%, or 99% sequence identity thereto, or a portion of any of the foregoing.

22. The fusion protein of any of embodiments 18-21, wherein the C-terminal Intein comprises a C-terminal Npu Intein, or the sequence set forth in SEQ ID NO:92, or an amino acid sequence that has at least 90%, 91%, 92%, 93%, 94%, 95%, 96%, 97%, 98%, or 99% sequence identity thereto, or a portion of any of the foregoing.

23. The fusion protein of any of embodiments 18-22, wherein the C-terminal fragment of the variant Cas protein comprises:
the C-terminal fragment of variant SpCas9 from position 574 to the C-terminal end of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or the sequence set forth in SEQ ID NO:94, or an amino acid sequence that has at least 90%, 91%, 92%, 93%, 94%, 95%, 96%, 97%, 98%, or 99% sequence identity thereto, or a portion of any of the foregoing.

24. The fusion protein of any of embodiments 4, 6, 7, or 10-23, wherein the Cas protein or a variant thereof is a Cpf1 or a variant thereof.

25. The fusion protein of any of embodiments 4, 6, and 7, wherein the Cas protein or a variant thereof is a variant Cpf1 that that is a deactivated Cpf1 (dCpf1).

26. The fusion protein of embodiment 24 or 25, wherein the variant comprises a catalytically inactive nuclease variant.

27. The fusion protein of any of embodiments 1-26, wherein the transcriptional activation domain of NCOA3 comprises:
(i) the sequence set forth in SEQ ID NO:40;
(ii) a contiguous portion of SEQ ID NO:40 of at least 20 amino acids;
(iii) the sequence set forth in SEQ ID NO:27;
(iv) a contiguous portion of SEQ ID NO:27 of at least 20 amino acids;
(v) an amino acid sequence that has at least 90%, 91%, 92%, 93%, 94%, 95%, 96%, 97%, 98%, or 99% sequence identity to any of the foregoing.

28. The fusion protein of any of embodiments 1-27, wherein the transcriptional activation domain of NCOA3 comprises the sequence set forth in SEQ ID NO:133 or an amino acid sequence that has at least 90%, 91%, 92%, 93%, 94%, 95%, 96%, 97%, 98%, or 99% sequence identity to SEQ ID NO:133.

29. The fusion protein of any of embodiments 1-28, wherein the transcriptional activation domain of NCOA3 comprises the sequence set forth in SEQ ID NO:133.

30. The fusion protein of any of embodiments 1-29, wherein the transcriptional activation domain of ENL comprises:
(i) the sequence set forth in SEQ ID NO:36;
(ii) a contiguous portion of SEQ ID NO:36 of at least 20 amino acids;
(iii) the sequence set forth in SEQ ID NO:23;
(iv) a contiguous portion of SEQ ID NO:23 of at least 20 amino acids;
(v) an amino acid sequence that has at least 90%, 91%, 92%, 93%, 94%, 95%, 96%, 97%, 98%, or 99% sequence identity to any of the foregoing.

31. The fusion protein of any of embodiments 1-30, wherein the transcriptional activation domain of ENL comprises the sequence set forth in SEQ ID NO:131 or an amino acid sequence that has at least 90%, 91%, 92%, 93%, 94%, 95%, 96%, 97%, 98%, or 99% sequence identity to any of the foregoing.

32. The fusion protein of any of embodiments 1-31, wherein the transcriptional activation domain of ENL comprises the sequence set forth in SEQ ID NO:131.

33. The fusion protein of any of embodiments 1-32, wherein the transcriptional activation domain of FOXO3 comprises:
(i) the sequence set forth in SEQ ID NO:37;
(ii) a contiguous portion of SEQ ID NO:37 of at least 20 amino acids;
(iii) the sequence set forth in SEQ ID NO:24;
(iv) a contiguous portion of SEQ ID NO:24 of at least 20 amino acids;
(v) an amino acid sequence that has at least 90%, 91%, 92%, 93%, 94%, 95%, 96%, 97%, 98%, or 99% sequence identity to any of the foregoing.

34. The fusion protein of any of embodiments 1-33, wherein the transcriptional activation domain of FOXO3 comprises the sequence set forth in SEQ ID NO:132 or an amino acid sequence that has at least 90%, 91%, 92%, 93%, 94%, 95%, 96%, 97%, 98%, or 99% sequence identity to any of the foregoing.

35. The fusion protein of any of embodiments 1-34, wherein the transcriptional activation domain of FOXO3 comprises the sequence set forth in SEQ ID NO:132.

36. The fusion protein of any of embodiments 1-35, wherein the transcriptional activation domain of PYGO1 comprises:
(i) the sequence set forth in SEQ ID NO:42;
(ii) a contiguous portion of SEQ ID NO:42 of at least 20 amino acids;
(iii) the sequence set forth in SEQ ID NO:29;
(iv) a contiguous portion of SEQ ID NO:29 of at least 20 amino acids;
(v) an amino acid sequence that has at least 90%, 91%, 92%, 93%, 94%, 95%, 96%, 97%, 98%, or 99% sequence identity to any of the foregoing.

37. The fusion protein of any of embodiments 1-36, wherein the transcriptional activation domain of PYGO1 comprises the sequence set forth in SEQ ID NO:130 or an amino acid sequence that has at least 90%, 91%, 92%, 93%, 94%, 95%, 96%, 97%, 98%, or 99% sequence identity to any of the foregoing.

38. The fusion protein of any of embodiments 1-37, wherein the transcriptional activation domain of PYGO1 comprises the sequence set forth in SEQ ID NO:130.

39. The fusion protein of any of embodiments 1-38, wherein the transcriptional activation domain of HSH2D comprises:
(i) the sequence set forth in SEQ ID NO:38;
(ii) a contiguous portion of SEQ ID NO:38 of at least 20 amino acids;
(iii) the sequence set forth in SEQ ID NO:25;
(iv) a contiguous portion of SEQ ID NO:25 of at least 20 amino acids;
(v) an amino acid sequence that has at least 90%, 91%, 92%, 93%, 94%, 95%, 96%, 97%, 98%, or 99% sequence identity to any of the foregoing.

40. The fusion protein of any of embodiments 1-39, wherein the transcriptional activation domain of HSH2D comprises the sequence set forth in SEQ ID NO:134 or an amino acid sequence that has at least 90%, 91%, 92%, 93%, 94%, 95%, 96%, 97%, 98%, or 99% sequence identity to any of the foregoing.

41. The fusion protein of any of embodiments 1-40, wherein the transcriptional activation domain of HSH2D comprises the sequence set forth in SEQ ID NO:134.

42. The fusion protein of any of embodiments 1-41, wherein the transcriptional activation domain of NCOA2 comprises:
(i) the sequence set forth in SEQ ID NO:39;
(ii) a contiguous portion of SEQ ID NO:39 of at least 20 amino acids;
(iii) the sequence set forth in SEQ ID NO:26;
(iv) a contiguous portion of SEQ ID NO:26 of at least 20 amino acids;
(v) an amino acid sequence that has at least 90%, 91%, 92%, 93%, 94%, 95%, 96%, 97%, 98%, or 99% sequence identity to any of the foregoing.

43. The fusion protein of any of embodiments 1-42, wherein the transcriptional activation domain of NCOA2 comprises the sequence set forth in SEQ ID NO:135 or an amino acid sequence that has at least 90%, 91%, 92%, 93%, 94%, 95%, 96%, 97%, 98%, or 99% sequence identity to any of the foregoing.

44. The fusion protein of any of embodiments 1-43, wherein the transcriptional activation domain of NCOA2 comprises the sequence set forth in SEQ ID NO:135.

45. The fusion protein of any of embodiments 1-44, wherein the transcriptional activation domain of NOTCH2 comprises:
(i) the sequence set forth in SEQ ID NO:46;
(ii) a contiguous portion of SEQ ID NO:46 of at least 20 amino acids;
(iii) the sequence set forth in SEQ ID NO:33;
(iv) a contiguous portion of SEQ ID NO:33 of at least 20 amino acids;
(v) an amino acid sequence that has at least 90%, 91%, 92%, 93%, 94%, 95%, 96%, 97%, 98%, or 99% sequence identity to any of the foregoing.

46. The fusion protein of any of embodiments 1-45, wherein the transcriptional activation domain of NOTCH2 comprises the sequence set forth in SEQ ID NO:136 or an amino acid sequence that has at least 90%, 91%, 92%, 93%, 94%, 95%, 96%, 97%, 98%, or 99% sequence identity to any of the foregoing.

47. The fusion protein of any of embodiments 1-46, wherein the transcriptional activation domain of NOTCH2 comprises the sequence set forth in SEQ ID NO:136.

48. The fusion protein of any of embodiments 1-47, wherein the transcriptional activation domain of DPOLA comprises:
(i) the sequence set forth in SEQ ID NO:35;
(ii) a contiguous portion of SEQ ID NO:35 of at least 20 amino acids;
(iii) the sequence set forth in SEQ ID NO:22;
(iv) a contiguous portion of SEQ ID NO:22 of at least 20 amino acids;
(v) an amino acid sequence that has at least 90%, 91%, 92%, 93%, 94%, 95%, 96%, 97%, 98%, or 99% sequence identity to any of the foregoing.

49. The fusion protein of any of embodiments 1-48, wherein the transcriptional activation domain of DPOLA comprises the sequence set forth in SEQ ID NO:176 or an amino acid sequence that has at least 90%, 91%, 92%, 93%, 94%, 95%, 96%, 97%, 98%, or 99% sequence identity to any of the foregoing.

50. The fusion protein of any of embodiments 1-49, wherein the transcriptional activation domain of DPOLA comprises the sequence set forth in SEQ ID NO:176.

51. The fusion protein of any of embodiments 1-50, wherein the transcriptional activation domain of PSA1 comprises:
(i) the sequence set forth in SEQ ID NO:41;
(ii) a contiguous portion of SEQ ID NO:41 of at least 20 amino acids;
(iii) the sequence set forth in SEQ ID NO:28;
(iv) a contiguous portion of SEQ ID NO:28 of at least 20 amino acids;
(v) an amino acid sequence that has at least 90%, 91%, 92%, 93%, 94%, 95%, 96%, 97%, 98%, or 99% sequence identity to any of the foregoing.

52. The fusion protein of any of embodiments 1-51, wherein the transcriptional activation domain of PSA1 comprises the sequence set forth in SEQ ID NO:177 or an amino acid sequence that has at least 90%, 91%, 92%, 93%, 94%, 95%, 96%, 97%, 98%, or 99% sequence identity to any of the foregoing.

53. The fusion protein of any of embodiments 1-52, wherein the transcriptional activation domain of PSA1 comprises the sequence set forth in SEQ ID NO:177.

54. The fusion protein of any of embodiments 1-53, wherein the transcriptional activation domain of RBM39 comprises:
(i) the sequence set forth in SEQ ID NO:43;
(ii) a contiguous portion of SEQ ID NO:43 of at least 20 amino acids;
(iii) the sequence set forth in SEQ ID NO:30;
(iv) a contiguous portion of SEQ ID NO:30 of at least 20 amino acids;
(v) an amino acid sequence that has at least 90%, 91%, 92%, 93%, 94%, 95%, 96%, 97%, 98%, or 99% sequence identity to any of the foregoing.

55. The fusion protein of any of embodiments 1-54, wherein the transcriptional activation domain of RBM39 comprises the sequence set forth in SEQ ID NO:178 or an amino acid sequence that has at least 90%, 91%, 92%, 93%, 94%, 95%, 96%, 97%, 98%, or 99% sequence identity to any of the foregoing.

56. The fusion protein of any of embodiments 1-55, wherein the transcriptional activation domain of RBM39 comprises the sequence set forth in SEQ ID NO:178.

57. The fusion protein of any of embodiments 1-56, wherein the transcriptional activation domain of HERC2 comprises:
(i) the sequence set forth in SEQ ID NO:44;
(ii) a contiguous portion of SEQ ID NO:44 of at least 20 amino acids;
(iii) the sequence set forth in SEQ ID NO:31;
(iv) a contiguous portion of SEQ ID NO:31 of at least 20 amino acids;
(v) an amino acid sequence that has at least 90%, 91%, 92%, 93%, 94%, 95%, 96%, 97%, 98%, or 99% sequence identity to any of the foregoing.

58. The fusion protein of any of embodiments 1-57, wherein the transcriptional activation domain of HERC2 comprises the sequence set forth in SEQ ID NO:179 or an amino acid sequence that has at least 90%, 91%, 92%, 93%, 94%, 95%, 96%, 97%, 98%, or 99% sequence identity to any of the foregoing.

59. The fusion protein of any of embodiments 1-58, wherein the transcriptional activation domain of HERC2 comprises the sequence set forth in SEQ ID NO:179.

60. The fusion protein of any of embodiments 1-59, wherein the transcriptional activator domain is at least at or about 30, 40, 50, 60, or 70 amino acids in length.

61. The fusion protein of any of embodiments 1-60, wherein the transcriptional activator domain is at least at or about 40 amino acids in length.

62. The fusion protein of any of embodiments 1-60, wherein the transcriptional activator domain is at least at or about 50 amino acids in length.

63. The fusion protein of any of embodiments 1-60, wherein the transcriptional activator domain is at least at or about 60 amino acids in length.

64. The fusion protein of any of embodiments 1-60, wherein the transcriptional activator domain is at least at or about 70 amino acids in length.

65. The fusion protein of any of embodiments 1-60, wherein the transcriptional activator domain is at or about 120, 110, 100, 90, 80, 70, 60, 50, or 40 amino acids or less in length.

66. The fusion protein of any of embodiments 1-60 and 65, wherein the transcriptional activator domain is 70 amino acids or less in length.

67. The fusion protein of any of embodiments 1-60 and 65, wherein the transcriptional activator domain is 60 amino acids or less in length.

68. The fusion protein of any of embodiments 1-60 and 65, wherein the transcriptional activator domain is 50 amino acids or less in length.

69. The fusion protein of any of embodiments 1-59, wherein the transcriptional activator domain is between at or about 40 and at or about 120, at or about 40 and at or about 110, at or about 40 and at or about 100, at or about 40 and at or about 90, at or about 40 and at or about 80, at or about 40 and at or about 70, at or about 40 and at or about 60, or at or about 40 and at or about 50 amino acids in length.

70. The fusion protein of any of embodiments 1-69, wherein the fusion protein comprises a multipartite effector comprising at least two of the two or more transcriptional activation domains.

71. The fusion protein of embodiment 70, wherein the multipartite effector is composed of two transcriptional activation domains.

72. The fusion protein of embodiment 70 or embodiment 71, wherein the multipartite effector is set forth in any of SEQ ID NOS:140-153, a portion thereof, or an amino acid sequence that has at least 90%, 91%, 92%, 93%, 94%, 95%, 96%, 97%, 98%, or 99% sequence identity to any of the foregoing

73. The fusion protein of any of embodiments 1-70, wherein the two or more transcriptional activation domains is two transcriptional activation domains.

74. The fusion protein of any of embodiments 1-73, wherein the fusion protein comprises:
a transcriptional activation domain of PYGO1 and a transcriptional activation domain of NCOA3;
a transcriptional activation domain of NOTCH2 and a transcriptional activation domain of NCOA3;
a transcriptional activation domain of NCOA3 and a transcriptional activation domain of NCOA3;
a transcriptional activation domain of HSH2D and a transcriptional activation domain of NCOA3;
a transcriptional activation domain of FOXO3 and a transcriptional activation domain of NCOA3;
a transcriptional activation domain of NCOA2 and a transcriptional activation domain of NCOA3;
a transcriptional activation domain of ENL and a transcriptional activation domain of NCOA3;
a transcriptional activation domain of PYGO1 and a transcriptional activation domain of FOXO3;
a transcriptional activation domain of NOTCH2 and a transcriptional activation domain of FOXO3;
a transcriptional activation domain of NCOA3 and a transcriptional activation domain of FOXO3;
a transcriptional activation domain of HSH2D and a transcriptional activation domain of FOXO3;
a transcriptional activation domain of FOXO3 and a transcriptional activation domain of FOXO3;
a transcriptional activation domain of NCOA2 and a transcriptional activation domain of FOXO3; or
a transcriptional activation domain of ENL and a transcriptional activation domain of FOXO3.

75. The fusion protein of any of embodiments 1-74, wherein the fusion protein comprises, in N-terminus to C-terminus order:
a transcriptional activation domain of PYGO1, a linker, and a transcriptional activation domain of NCOA3;
a transcriptional activation domain of NOTCH2, a linker, and a transcriptional activation domain of NCOA3;
a transcriptional activation domain of NCOA3, a linker, and a transcriptional activation domain of NCOA3;
a transcriptional activation domain of HSH2D, a linker, and a transcriptional activation domain of NCOA3;
a transcriptional activation domain of FOXO3, a linker, and a transcriptional activation domain of NCOA3;
a transcriptional activation domain of NCOA2, a linker, and a transcriptional activation domain of NCOA3;
a transcriptional activation domain of ENL, a linker, and a transcriptional activation domain of NCOA3;
a transcriptional activation domain of PYGO1, a linker, and a transcriptional activation domain of FOXO3;
a transcriptional activation domain of NOTCH2, a linker, and a transcriptional activation domain of FOXO3;
a transcriptional activation domain of NCOA3, a linker, and a transcriptional activation domain of FOXO3;
a transcriptional activation domain of HSH2D, a linker, and a transcriptional activation domain of FOXO3;
a transcriptional activation domain of FOXO3, a linker, and a transcriptional activation domain of FOXO3;
a transcriptional activation domain of NCOA2, a linker, and a transcriptional activation domain of FOXO3; or
a transcriptional activation domain of ENL, a linker, and a transcriptional activation domain of FOXO3.

76. The fusion protein of any of embodiments 1-74, wherein the fusion protein comprises the sequence set forth in any one of SEQ ID NOS:140-153, or an amino acid sequence that has at least 90%, 91%, 92%, 93%, 94%, 95%, 96%, 97%, 98%, or 99% sequence identity to any of the foregoing, optionally wherein the fusion protein comprises the sequence set forth in SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, or SEQ ID NO:153.

77. The fusion protein of any of embodiments 1-76, wherein the fusion protein comprises, in N-terminus to C-terminus order:
a dCas, optionally a dCas9, a transcriptional activation domain of PYGO1 and a transcriptional activation domain of NCOA3;
a dCas, optionally a dCas9, a transcriptional activation domain of NOTCH2 and a transcriptional activation domain of NCOA3;
a dCas, optionally a dCas9, a transcriptional activation domain of NCOA3 and a transcriptional activation domain of NCOA3;
a dCas, optionally a dCas9, a transcriptional activation domain of HSH2D and a transcriptional activation domain of NCOA3;
a dCas, optionally a dCas9, a transcriptional activation domain of FOXO3 and a transcriptional activation domain of NCOA3;
a dCas, optionally a dCas9, a transcriptional activation domain of NCOA2 and a transcriptional activation domain of NCOA3;
a dCas, optionally a dCas9, a transcriptional activation domain of ENL and a transcriptional activation domain of NCOA3;
a dCas, optionally a dCas9, a transcriptional activation domain of PYGO1 and a transcriptional activation domain of FOXO3;
a dCas, optionally a dCas9, a transcriptional activation domain of NOTCH2 and a transcriptional activation domain of FOXO3;
a dCas, optionally a dCas9, a transcriptional activation domain of NCOA3 and a transcriptional activation domain of FOXO3;
a dCas, optionally a dCas9, a transcriptional activation domain of HSH2D and a transcriptional activation domain of FOXO3;
a dCas, optionally a dCas9, a transcriptional activation domain of FOXO3 and a transcriptional activation domain of FOXO3;
a dCas, optionally a dCas9, a transcriptional activation domain of NCOA2 and a transcriptional activation domain of FOXO3; or
a dCas, optionally a dCas9, a transcriptional activation domain of ENL and a transcriptional activation domain of FOXO3.

78. The fusion protein of any of embodiments 1-76, wherein the fusion protein comprises, in N-terminus to C-terminus order:
a ZFP, a transcriptional activation domain of PYGO1 and a transcriptional activation domain of NCOA3;
a ZFP, a transcriptional activation domain of NOTCH2 and a transcriptional activation domain of NCOA3;
a ZFP, a transcriptional activation domain of NCOA3 and a transcriptional activation domain of NCOA3;
a ZFP, a transcriptional activation domain of HSH2D and a transcriptional activation domain of NCOA3;
a ZFP, a transcriptional activation domain of FOXO3 and a transcriptional activation domain of NCOA3;
a ZFP, a transcriptional activation domain of NCOA2 and a transcriptional activation domain of NCOA3;
a ZFP, a transcriptional activation domain of ENL and a transcriptional activation domain of NCOA3;
a ZFP, a transcriptional activation domain of PYGO1 and a transcriptional activation domain of FOXO3;
a ZFP, a transcriptional activation domain of NOTCH2 and a transcriptional activation domain of FOXO3;
a ZFP, a transcriptional activation domain of NCOA3 and a transcriptional activation domain of FOXO3;
a ZFP, a transcriptional activation domain of HSH2D and a transcriptional activation domain of FOXO3;
a ZFP, a transcriptional activation domain of FOXO3 and a transcriptional activation domain of FOXO3;
a ZFP, a transcriptional activation domain of NCOA2 and a transcriptional activation domain of FOXO3; or
a ZFP, a transcriptional activation domain of ENL and a transcriptional activation domain of FOXO3.

79. The fusion protein of any of embodiments 1-76, wherein the fusion protein comprises, in N-terminus to C-terminus order:
a transcriptional activation domain of PYGO1, a transcriptional activation domain of NCOA3, and a dCas, optionally a dCas9;
a transcriptional activation domain of NOTCH2, a transcriptional activation domain of NCOA3, and a dCas, optionally a dCas9;
a transcriptional activation domain of NCOA3, a transcriptional activation domain of NCOA3, and a dCas, optionally a dCas9;
a transcriptional activation domain of HSH2D, a transcriptional activation domain of NCOA3, and a dCas, optionally a dCas9;
a transcriptional activation domain of FOXO3, a transcriptional activation domain of NCOA3, and a dCas, optionally a dCas9;
a transcriptional activation domain of NCOA2, a transcriptional activation domain of NCOA3, and a dCas, optionally a dCas9;
a transcriptional activation domain of ENL, a transcriptional activation domain of NCOA3, and a dCas, optionally a dCas9;
a transcriptional activation domain of PYGO1, a transcriptional activation domain of FOXO3, and a dCas, optionally a dCas9;
a transcriptional activation domain of NOTCH2, a transcriptional activation domain of FOXO3, and a dCas, optionally a dCas9;
a transcriptional activation domain of NCOA3, a transcriptional activation domain of FOXO3, and a dCas, optionally a dCas9;
a transcriptional activation domain of HSH2D, a transcriptional activation domain of FOXO3, and a dCas, optionally a dCas9;
a transcriptional activation domain of FOXO3, a transcriptional activation domain of FOXO3, and a dCas, optionally a dCas9;
a transcriptional activation domain of NCOA2, a transcriptional activation domain of FOXO3, and a dCas, optionally a dCas9; or
a transcriptional activation domain of ENL, a transcriptional activation domain of FOXO3, and a dCas, optionally a dCas9.

80. The fusion protein of any of embodiments 1-76, wherein the fusion protein comprises, in N-terminus to C-terminus order:
a transcriptional activation domain of PYGO1, a transcriptional activation domain of NCOA3, and a ZFP;
a transcriptional activation domain of NOTCH2, a transcriptional activation domain of NCOA3, and a ZFP;
a transcriptional activation domain of NCOA3, a transcriptional activation domain of NCOA3, and a ZFP;
a transcriptional activation domain of HSH2D, a transcriptional activation domain of NCOA3, and a ZFP;
a transcriptional activation domain of FOXO3, a transcriptional activation domain of NCOA3, and a ZFP;
a transcriptional activation domain of NCOA2, a transcriptional activation domain of NCOA3, and a ZFP;
a transcriptional activation domain of ENL, a transcriptional activation domain of NCOA3, and a ZFP;
a transcriptional activation domain of PYGO1, a transcriptional activation domain of FOXO3, and a ZFP;
a transcriptional activation domain of NOTCH2, a transcriptional activation domain of FOXO3, and a ZFP;
a transcriptional activation domain of NCOA3, a transcriptional activation domain of FOXO3, and a ZFP;
a transcriptional activation domain of HSH2D, a transcriptional activation domain of FOXO3, and a ZFP;
a transcriptional activation domain of FOXO3, a transcriptional activation domain of FOXO3, and a ZFP;
a transcriptional activation domain of NCOA2, a transcriptional activation domain of FOXO3, and a ZFP; or
a transcriptional activation domain of ENL, a transcriptional activation domain of FOXO3, and a ZFP.

81. The fusion protein of any of embodiments 1-70 and 74-80, wherein the two or more transcriptional activation domains is three transcriptional activation domains.

82. The fusion protein of any of embodiments 1-70 and 81, wherein the fusion protein comprises a multipartite effector comprising at least three transcriptional activation domains.

83. The fusion protein of embodiment 70 and embodiment 82, wherein the multipartite effector is composed of two transcriptional activation domains or three transcriptional activation domains.

84. The fusion protein of embodiment 70, 82 or 83, wherein the multipartite effector is set forth in any of SEQ ID NOS:154-160 and 377, a portion thereof, or an amino acid sequence that has at least 90%, 91%, 92%, 93%, 94%, 95%, 96%, 97%, 98%, or 99% sequence identity to any of the foregoing.

85. The fusion protein of any of embodiments 1-70 and 74-84, wherein the fusion protein comprises:
a transcriptional activation domain of PYGO1, a transcriptional activation domain of FOXO3, and a transcriptional activation domain of NCOA3;
a transcriptional activation domain of NOTCH2, a transcriptional activation domain of FOXO3, and a transcriptional activation domain of NCOA3;
a transcriptional activation domain of NCOA3, a transcriptional activation domain of FOXO3, and a transcriptional activation domain of NCOA3;
a transcriptional activation domain of HSH2D, a transcriptional activation domain of FOXO3, and a transcriptional activation domain of NCOA3;
a transcriptional activation domain of FOXO3, a transcriptional activation domain of FOXO3, and a transcriptional activation domain of NCOA3;
a transcriptional activation domain of NCOA2, a transcriptional activation domain of FOXO3, and a transcriptional activation domain of NCOA3;
a transcriptional activation domain of ENL, a transcriptional activation domain of FOXO3, and a transcriptional activation domain of NCOA3; or
a transcriptional activation domain of NCOA3, a transcriptional activation domain of FOXO3, and a transcriptional activation domain of FOXO3.

86. The fusion protein of any of embodiments 1-70 and 74-85, wherein the fusion protein comprises, in N-terminus to C-terminus order:
a transcriptional activation domain of PYGO1, a linker, a transcriptional activation domain of FOXO3, a linker, and a transcriptional activation domain of NCOA3;
a transcriptional activation domain of NOTCH2, a linker, a transcriptional activation domain of FOXO3, a linker, and a transcriptional activation domain of NCOA3;
a transcriptional activation domain of NCOA3, a linker, a transcriptional activation domain of FOXO3, a linker, and a transcriptional activation domain of NCOA3;
a transcriptional activation domain of HSH2D, a linker, a transcriptional activation domain of FOXO3, a linker, and a transcriptional activation domain of NCOA3;
a transcriptional activation domain of FOXO3, a linker, a transcriptional activation domain of FOXO3, a linker, and a transcriptional activation domain of NCOA3;
a transcriptional activation domain of NCOA2, a linker, a transcriptional activation domain of FOXO3, a linker, and a transcriptional activation domain of NCOA3;
a transcriptional activation domain of ENL, a linker, a transcriptional activation domain of FOXO3, a linker, and a transcriptional activation domain of NCOA3; or
a transcriptional activation domain of NCOA3, a linker, a transcriptional activation domain of FOXO3, a linker, and a transcriptional activation domain of FOXO3.

87. The fusion protein of any of embodiments 1-70 and 74-86, wherein the fusion protein comprises the sequence set forth in any one of SEQ ID NOS:154-160 and 377 or an amino acid sequence that has at least 90%, 91%, 92%, 93%, 94%, 95%, 96%, 97%, 98%, or 99% sequence identity to any of the foregoing.

88. The fusion protein of any of embodiments 1-70 and 74-87, wherein the fusion protein comprises the sequence set forth in SEQ ID NO:154, SEQ ID NO:155, SEQ ID NO:156, SEQ ID NO:157, SEQ ID NO:158, SEQ ID NO:159, or SEQ ID NO:160 or SEQ ID NO: 377.

89. The fusion protein of any of embodiments 70 and 82-88, wherein the multipartite effector comprises, in the N-terminal to C-terminal direction, domains from FOXO3, FOXO3, and NCOA3, respectively.

90. The fusion protein of embodiment 89, wherein the multipartite effector comprises the sequence set forth in SEQ ID NO:158, or an amino acid sequence that has at least 90%, 91%, 92%, 93%, 94%, 95%, 96%, 97%, 98%, or 99% sequence identity thereto.

91. The fusion protein of any of embodiments 70 and 82-88, wherein the multipartite effector comprises, in the N-terminal to C-terminal direction, domains from NCOA3, FOXO3, and NCOA3, respectively

92. The fusion protein of embodiment 91, wherein the multipartite effector comprises the sequence set forth in SEQ ID NO:156, or an amino acid sequence that has at least 90%, 91%, 92%, 93%, 94%, 95%, 96%, 97%, 98%, or 99% sequence identity thereto

93. The fusion protein of any of embodiments 70 and 82-88, wherein the multipartite effector comprises, in the N-terminal to C-terminal direction, domains from NCOA2, FOXO3, and NCOA3, respectively

94. The fusion protein of embodiment 93, wherein the multipartite effector comprises the sequence set forth in SEQ ID NO:159, or an amino acid sequence that has at least 90%, 91%, 92%, 93%, 94%, 95%, 96%, 97%, 98%, or 99% sequence identity thereto

95. The fusion protein of any of embodiments 70 and 82-88, wherein the multipartite effector comprises, in the N-terminal to C-terminal direction, domains from PYGO1, FOXO3, and NCOA3, respectively

96. The fusion protein of embodiment 95, wherein the multipartite effector comprises the sequence set forth in SEQ ID NO:154, or an amino acid sequence that has at least 90%, 91%, 92%, 93%, 94%, 95%, 96%, 97%, 98%, or 99% sequence identity thereto

97. The fusion protein of any of embodiments 70 and 82-88, wherein the multipartite effector comprises, in the N-terminal to C-terminal direction, domains from NCOA3, FOXO3, and FOXO3, respectively

98. The fusion protein of embodiment 97, wherein the multipartite effector comprises the sequence set forth in SEQ ID NO:377, or an amino acid sequence that has at least 90%, 91%, 92%, 93%, 94%, 95%, 96%, 97%, 98%, or 99% sequence identity thereto

99. The fusion protein of any of embodiments 1-70 and 74-88, wherein the fusion protein comprises a transcriptional activation domain of PYGO1, a linker, a transcriptional activation domain of FOXO3, a linker, and a transcriptional activation domain of NCOA3.

100. The fusion protein of any of embodiments 1-70, 74-88, 95, 96, 99, wherein the fusion protein comprises the sequence set forth in SEQ ID NO:154.

101. The fusion protein of any of embodiments 1-70 and 74-88, wherein the fusion protein comprises a transcriptional activation domain of NCOA3, a linker, a transcriptional activation domain of FOXO3, a linker, and a transcriptional activation domain of NCOA3.

102. The fusion protein of any of embodiments 1-70, 74-88, 91, 92, and 101, wherein the fusion protein comprises the sequence set forth in SEQ ID NO:156.

103. The fusion protein of any of embodiments 1-70, 74-88, wherein the fusion protein comprises a transcriptional activation domain of FOXO3, a linker, a transcriptional activation domain of FOXO3, a linker, and a transcriptional activation domain of NCOA3.

104. The fusion protein of any of embodiments 1-70, 74-88, 89, 90 and 103, wherein the fusion protein comprises the sequence set forth in SEQ ID NO:158.

105. The fusion protein of any of embodiments 1-70 and 74-88, wherein the fusion protein comprises a transcriptional activation domain of NCOA2, a linker, a transcriptional activation domain of FOXO3, a linker, and a transcriptional activation domain of NCOA3.

106. The fusion protein of any of embodiments 1-70, 74-88, 93, 94 and 105, wherein the fusion protein comprises the sequence set forth in SEQ ID NO:159.

107. The fusion protein of any of embodiments 1-70 and 74-88, wherein the fusion protein comprises, in N-terminus to C-terminus order:
a dCas, optionally a dCas9, a linker, a transcriptional activation domain of PYGO1, a linker, a transcriptional activation domain of FOXO3, a linker, and a transcriptional activation domain of NCOA3;
a dCas, optionally a dCas9, a linker, a transcriptional activation domain of NOTCH2, a linker, a transcriptional activation domain of FOXO3, a linker, and a transcriptional activation domain of NCOA3;
a dCas, optionally a dCas9, a linker, a transcriptional activation domain of NCOA3, a linker, a transcriptional activation domain of FOXO3, a linker, and a transcriptional activation domain of NCOA3;
a dCas, optionally a dCas9, a linker, a transcriptional activation domain of HSH2D, a linker, a transcriptional activation domain of FOXO3, a linker, and a transcriptional activation domain of NCOA3;
a dCas, optionally a dCas9, a linker, a transcriptional activation domain of FOXO3, a linker, a transcriptional activation domain of FOXO3, a linker, and a transcriptional activation domain of NCOA3;
a dCas, optionally a dCas9, a linker, a transcriptional activation domain of NCOA2, a linker, a transcriptional activation domain of FOXO3, a linker, and a transcriptional activation domain of NCOA3;
a dCas, optionally a dCas9, a linker, a transcriptional activation domain of ENL, a linker, a transcriptional activation domain of FOXO3, a linker, and a transcriptional activation domain of NCOA3; or
a dCas, optionally a dCas9, a linker, a transcriptional activation domain of NCOA3, a linker, a transcriptional activation domain of FOXO3, a linker, and a transcriptional activation domain of FOXO3.

108. The fusion protein of any of embodiments 1-70 and 74-88, wherein the fusion protein comprises, in N-terminus to C-terminus order:
a ZFP, a linker, a transcriptional activation domain of PYGO1, a linker, a transcriptional activation domain of FOXO3, a linker, and a transcriptional activation domain of NCOA3; a ZFP, a linker, a transcriptional activation domain of NOTCH2, a linker, a transcriptional activation domain of FOXO3, a linker, and a transcriptional activation domain of NCOA3;
a ZFP, a linker, a transcriptional activation domain of NCOA3, a linker, a transcriptional activation domain of FOXO3, a linker, and a transcriptional activation domain of NCOA3;
a ZFP, a linker, a transcriptional activation domain of HSH2D, a linker, a transcriptional activation domain of FOXO3, a linker, and a transcriptional activation domain of NCOA3;
a ZFP, a linker, a transcriptional activation domain of FOXO3, a linker, a transcriptional activation domain of FOXO3, a linker, and a transcriptional activation domain of NCOA3;
a ZFP, a linker, a transcriptional activation domain of NCOA2, a linker, a transcriptional activation domain of FOXO3, a linker, and a transcriptional activation domain of NCOA3;
a ZFP, a linker, a transcriptional activation domain of ENL, a linker, a transcriptional activation domain of FOXO3, a linker, and a transcriptional activation domain of NCOA3; or
a ZFP, a linker, a transcriptional activation domain of NCOA3, a linker, a transcriptional activation domain of FOXO3, a linker, and a transcriptional activation domain of FOXO3.

109. The fusion protein of any of embodiments 1-70 and 74-88, wherein the fusion protein comprises, in N-terminus to C-terminus order:
a transcriptional activation domain of PYGO1, a linker, a transcriptional activation domain of FOXO3, a linker, a transcriptional activation domain of NCOA3, a linker, and a dCas, optionally a dCas9;
a transcriptional activation domain of NOTCH2, a linker, a transcriptional activation domain of FOXO3, a linker, a transcriptional activation domain of NCOA3, a linker, and a dCas, optionally a dCas9;
a transcriptional activation domain of NCOA3, a linker, a transcriptional activation domain of FOXO3, a linker, a transcriptional activation domain of NCOA3, a linker, and a dCas, optionally a dCas9;
a transcriptional activation domain of HSH2D, a linker, a transcriptional activation domain of FOXO3, a linker, a transcriptional activation domain of NCOA3, a linker, and a dCas, optionally a dCas9;
a transcriptional activation domain of FOXO3, a linker, a transcriptional activation domain of FOXO3, a linker, a transcriptional activation domain of NCOA3, a linker, and a dCas, optionally a dCas9;
a transcriptional activation domain of NCOA2, a linker, a transcriptional activation domain of FOXO3, a linker, a transcriptional activation domain of NCOA3, a linker, and a dCas, optionally a dCas9;
a transcriptional activation domain of ENL, a linker, a transcriptional activation domain of FOXO3, a linker, a transcriptional activation domain of NCOA3, a linker, and a dCas, optionally a dCas9; or
a transcriptional activation domain of NCOA3, a linker, a transcriptional activation domain of FOXO3, a linker, a transcriptional activation domain of FOXO3, a linker, and a dCas, optionally a dCas9.

110. The fusion protein of any of embodiments 1-70 and 74-88, wherein the fusion protein comprises, in N-terminus to C-terminus order:
a transcriptional activation domain of PYGO1, a linker, a transcriptional activation domain of FOXO3, a linker, a transcriptional activation domain of NCOA3, a linker, and a ZFP;
a transcriptional activation domain of NOTCH2, a linker, a transcriptional activation domain of FOXO3, a linker, a transcriptional activation domain of NCOA3, a linker, and a ZFP;
a transcriptional activation domain of NCOA3, a linker, a transcriptional activation domain of FOXO3, a linker, a transcriptional activation domain of NCOA3, a linker, and a ZFP;
a transcriptional activation domain of HSH2D, a linker, a transcriptional activation domain of FOXO3, a linker, a transcriptional activation domain of NCOA3, a linker, and a ZFP;
a transcriptional activation domain of FOXO3, a linker, a transcriptional activation domain of FOXO3, a linker, a transcriptional activation domain of NCOA3, a linker, and a ZFP;
a transcriptional activation domain of NCOA2, a linker, a transcriptional activation domain of FOXO3, a linker, a transcriptional activation domain of NCOA3, a linker, and a ZFP; or
a transcriptional activation domain of ENL, a linker, a transcriptional activation domain of FOXO3, a linker, a transcriptional activation domain of NCOA3, a linker, and a ZFP; or
a transcriptional activation domain of NCOA3, a linker, a transcriptional activation domain of FOXO3, a linker, a transcriptional activation domain of FOXO3, a linker, and a ZFP.

111. The fusion protein of any of embodiments 1-70, 74, 75, 85, and 86, wherein the two or more transcriptional activation domains comprises four transcriptional activation domains.

112. The fusion protein of any of embodiments 1-70, 74, 75, 85, and 86, wherein the two or more transcriptional activation domains comprises five transcriptional activation domains.

113. The fusion protein of any of embodiments 1-112, wherein the fusion protein further comprises one or more linkers.

114. The fusion protein of embodiment 113, wherein a linker of the one or more linkers is positioned between the two or more transcriptional activation domains and/or positioned between the polypeptide component of the DNA-targeting domain and one of the two or more transcriptional activation domains.

115. The fusion protein of embodiment 113 or embodiment 114, wherein the linker is a polypeptide linker.

116. The fusion protein of embodiment 115, wherein the polypeptide linker comprises a sequence selected from among SEQ ID NOS:62-67, 96, and 137-139.

117. The fusion protein of any of embodiments 1-116, wherein the fusion protein further comprises one or more nuclear localization signals (NLSs).

118. The fusion protein of embodiment 117, wherein the one or more NLSs comprises two or more NLSs.

119. The fusion protein of embodiment 117 or embodiment 118, wherein a NLS of one or more NLSs is positioned between the two or more transcriptional activation domains.

120. The fusion protein of any of embodiments 117-119, wherein a NLS of the one or more NLSs is positioned between the polypeptide component of the DNA-targeting domain and one of the two or more transcriptional activation domains.

121. The fusion protein of any of embodiments 117-120, wherein the one or more NLSs comprises a sequence selected from among SEQ ID NOS:69-84.

122. The fusion protein of any of embodiments 1-88 and 107, 109 and 111-121, wherein the fusion protein comprises the sequence set forth in any one of SEQ ID NOS:181-187, or an amino acid sequence that has at least 90%, 91%, 92%, 93%, 94%, 95%, 96%, 97%, 98%, or 99% sequence identity to any of the foregoing.

123. The fusion protein of any of embodiments 1-88 and 107, 109 and 111-122, wherein the fusion protein comprises the sequence set forth in SEQ ID NO:181, SEQ ID NO:182, SEQ ID NO:183, SEQ ID NO:184, SEQ ID NO:185, SEQ ID NO:186, or SEQ ID NO:187.

124. The fusion protein of any of embodiments 1-123, further comprising a tag.

125. The fusion protein of embodiment 124, wherein the tag comprises an epitope tag or a split protein tag.

126. The fusion protein of embodiment 124 or embodiment 125, wherein the tag is selected from among SEQ ID NOS:61, 88, 92, and 167.

127. The fusion protein of any of embodiments 1-88 and 107, 109 and 111-126, wherein the fusion protein comprises the sequence set forth in any one of SEQ ID NOS:272-278, or an amino acid sequence that has at least 90%, 91%, 92%, 93%, 94%, 95%, 96%, 97%, 98%, or 99% sequence identity to any of the foregoing.

128. The fusion protein of any of embodiments 1-88 and 107, 109 and 111-127, wherein the fusion protein comprises the sequence set forth in SEQ ID NO:272, SEQ ID NO:273, SEQ ID NO:274, SEQ ID NO:275, SEQ ID NO:276, SEQ ID NO:277, or SEQ ID NO:278.

129. The fusion protein of any of embodiments 1-128, wherein the DNA-targeting domain is capable of hybridizing to the target site or is complementary to the target site at the endogenous locus.

130. The fusion protein of any of embodiments 1-129, wherein the at least one gRNA is capable of hybridizing to the target site or is complementary to the target site at the endogenous locus.

131. The fusion protein of embodiment 129 or claim 130, wherein the target site is located at a regulatory DNA element of the endogenous locus.

132. The fusion protein of embodiment 131, wherein the regulatory DNA element is selected from among a promoter, an upstream regulatory element, an enhancer, an exon, an intron, a 5′ untranslated region (UTR), a 3′ UTR, or a downstream regulatory element.

133. The fusion protein of any of embodiments 129-132, wherein the endogenous locus is FXN.

134. The fusion protein of any of embodiments 129-133, wherein the target site is located within the genomic coordinates hg38 chr9:68,940,179-69,205,519 or hg38 chr9:69,027,282-69,028,497. 135. The fusion protein of any of embodiments 129-134, wherein the target site is located within the genomic coordinates hg38 chr9:69,027,615-69,028,101.

136. The fusion protein of any of embodiments 129-134, wherein the target site comprises a sequence set forth in any one of SEQ ID NOS:208-228.

137. The fusion protein of any of embodiments 129-134 and 136, wherein the target site comprises a sequence set forth in SEQ ID NO:208.

138. The fusion protein of any of embodiments 129-134 and 136, wherein the target site comprises a sequence set forth in SEQ ID NO:214.

139. The fusion protein of any of embodiments 129-136, wherein the target site comprises a sequence set forth in SEQ ID NO:228.

140. A DNA-targeting system comprising the fusion protein of any of embodiments 1-139.

141. A DNA-targeting system comprising the fusion protein of any of embodiments 1-139, and at least one gRNA.

142. A DNA-targeting system comprising:
(1) a DNA-targeting domain, and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2, wherein the transcriptional activation domain increases transcription of an endogenous locus when recruited to a target site at the endogenous locus.

143. The DNA-targeting system of embodiment 142, wherein the DNA-targeting domain comprises a Cas-gRNA combination comprising a Cas protein or a variant thereof, and at least one gRNA that binds to the target site at the endogenous locus.

144. The DNA-targeting system of embodiment 143, wherein the DNA-targeting domain comprises a zinc finger protein (ZFP), a transcription activator-like effector (TALE), a meganuclease, a homing endonuclease, or an I-SceI enzyme or a variant thereof that binds to the target site at the endogenous locus.

145. A DNA-targeting system comprising:
(1) a Cas-gRNA combination comprising a Cas protein or a variant thereof, and at least one gRNA that binds to the target site at an endogenous locus, and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2, wherein the transcriptional activation domain increases transcription of an endogenous locus when recruited to a target site at the endogenous locus.

146. A DNA-targeting system comprising:
(1) a zinc finger protein (ZFP), a transcription activator-like effector (TALE), a meganuclease, a homing endonuclease, or an I-SceI enzyme or a variant thereof that binds to the target site at an endogenous locus, and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2, wherein the transcriptional activation domain increases transcription of an endogenous locus when recruited to a target site at the endogenous locus.

147. A DNA-targeting system comprising:
(1) a zinc finger protein (ZFP) that binds to the target site at an endogenous locus, and (2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, and HERC2, wherein the transcriptional activation domain increases transcription of an endogenous locus when recruited to a target site at the endogenous locus.

148. The DNA-targeting system of embodiment 143 or 145, wherein the Cas protein or a variant thereof, and the two or more transcriptional activation domains are fused in a fusion protein.

149. The DNA-targeting system of embodiment 144 or 146, wherein the zinc finger protein (ZFP), a transcription activator-like effector (TALE), a meganuclease, a homing endonuclease, or an I-SceI enzyme or a variant thereof, and the two or more transcriptional activation domains are fused in a fusion protein.

150. The DNA-targeting system of embodiment 144 or 147, wherein the zinc finger protein (ZFP) and the two or more transcriptional activation domains are fused in a fusion protein.

151. The DNA-targeting system of any of embodiments 143-149, wherein the variant thereof comprises a catalytically inactive variant.

152. The DNA-targeting system of any of embodiments 143, 145, 148, and 151, wherein the Cas protein or a variant thereof is a Cas9 or a variant thereof.

153. The DNA-targeting system of any of embodiments 143, 145, 148, 151, and 152, wherein the Cas protein or a variant thereof is a deactivated Cas9 (dCas9).

154. The DNA-targeting system of any of embodiments 143, 145, 148, and 151-153, wherein the Cas protein or a variant thereof is a Staphylococcus aureus Cas9 (SaCas9) or a variant thereof.

155. The DNA-targeting system of any of embodiments 143, 145, 148, and 151-154, wherein the Cas protein or a variant thereof is a Staphylococcus aureus dCas9 (dSaCas9) that comprises at least one amino acid mutation selected from D10A and N580A, with reference to numbering of positions of SEQ ID NO:3.

156. The DNA-targeting system of any of embodiments 143, 145, 148, and 151-155, wherein the Cas protein or a variant thereof comprises the sequence set forth in SEQ ID NO:2, or an amino acid sequence that has at least 90%, 91%, 92%, 93%, 94%, 95%, 96%, 97%, 98%, or 99% sequence identity thereto.

157. The DNA-targeting system of any of embodiments 143, 145, 148, and 151-153, wherein the Cas protein or a variant thereof is a Streptococcus pyogenes Cas9 (SpCas9) or a variant thereof.

158. The DNA-targeting system of any of embodiments 143, 145, 148, 151-153 and 157, wherein the Cas protein or a variant thereof is a Streptococcus pyogenes dCas9 (dSpCas9) that comprises at least one amino acid mutation selected from D10A and H840A, with reference to numbering of positions of SEQ ID NO:7.

159. The DNA-targeting system of any of embodiments 143, 145, 148, 151-153, 157 and 158, wherein the Cas protein or a variant thereof comprises the sequence set forth in SEQ ID NO:6, or an amino acid sequence that has at least 90%, 91%, 92%, 93%, 94%, 95%, 96%, 97%, 98%, or 99% sequence identity thereto.

160. The DNA-targeting system of any of embodiments 143, 145, 148, and 151-159, wherein the Cas protein or a variant thereof protein is a split variant Cas protein, wherein the split variant Cas protein comprises a first polypeptide comprising an N-terminal fragment of the variant Cas protein and an N-terminal Intein, and a second polypeptide comprising a C-terminal fragment of the variant Cas protein and a C-terminal Intein.

161. The DNA-targeting system of embodiment 160, wherein when the first polypeptide and the second polypeptide of the split variant Cas protein are present in proximity or present in the same cell, the N-terminal Intein and C-terminal Intein self-excise and ligate the N-terminal fragment and the C-terminal fragment of the variant Cas protein to form a full-length variant Cas protein.

162. The DNA-targeting system of embodiment 160 or 161, wherein the N-terminal Intein comprises an N-terminal Npu Intein, or the sequence set forth in SEQ ID NO:88, or an amino acid sequence that has at least 90%, 91%, 92%, 93%, 94%, 95%, 96%, 97%, 98%, or 99% sequence identity thereto, or a portion of any of the foregoing.

163. The DNA-targeting system of any of embodiments 160-162, wherein the N-terminal fragment of the variant Cas protein comprises:
the N-terminal fragment of variant SpCas9 from the N-terminal end up to position 573 of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or the sequence set forth in SEQ ID NO:86, or an amino acid sequence that has at least 90%, 91%, 92%, 93%, 94%, 95%, 96%, 97%, 98%, or 99% sequence identity thereto, or a portion of any of the foregoing.

164. The DNA-targeting system of any of embodiments 160-163, wherein the C-terminal Intein comprises a C-terminal Npu Intein, or the sequence set forth in SEQ ID NO:92, or an amino acid sequence that has at least 90%, 91%, 92%, 93%, 94%, 95%, 96%, 97%, 98%, or 99% sequence identity thereto, or a portion of any of the foregoing.

165. The DNA-targeting system of any of embodiments 160-164, wherein the C-terminal fragment of the variant Cas protein comprises:
the C-terminal fragment of variant SpCas9 from position 574 to the C-terminal end of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or the sequence set forth in SEQ ID NO:94, or an amino acid sequence that has at least 90%, 91%, 92%, 93%, 94%, 95%, 96%, 97%, 98%, or 99% sequence identity thereto, or a portion of any of the foregoing.

166. The DNA-targeting system of any of embodiments 143, 145, 148, and 151, wherein the Cas protein or a variant thereof is a Cpf1 or a variant thereof.

167. The DNA-targeting system of any of embodiments 143, 145, 148, 151 and 166, wherein the Cas protein or a variant thereof is a variant Cpf1 that that is a deactivated Cpf1 (dCpf1).

168. The DNA-targeting system of embodiment 166 or 167, wherein the variant comprises a catalytically inactive nuclease variant.

169. The DNA-targeting system of any of embodiments 142-168, wherein the transcriptional activation domain of NCOA3 comprises:
(i) the sequence set forth in SEQ ID NO:40;
(ii) a contiguous portion of SEQ ID NO:40 of at least 20 amino acids;
(iii) the sequence set forth in SEQ ID NO:27;
(iv) a contiguous portion of SEQ ID NO:27 of at least 20 amino acids;
(v) an amino acid sequence that has at least 90%, 91%, 92%, 93%, 94%, 95%, 96%, 97%, 98%, or 99% sequence identity to any of the foregoing.

170. The DNA-targeting system of any of embodiments 142-169, wherein the transcriptional activation domain of NCOA3 comprises the sequence set forth in SEQ ID NO:133 or an amino acid sequence that has at least 90%, 91%, 92%, 93%, 94%, 95%, 96%, 97%, 98%, or 99% sequence identity to SEQ ID NO:133.

171. The DNA-targeting system of any of embodiments 142-170, wherein the transcriptional activation domain of NCOA3 comprises the sequence set forth in SEQ ID NO:133.

172. The DNA-targeting system of any of embodiments 142-171, wherein the transcriptional activation domain of ENL comprises:
(i) the sequence set forth in SEQ ID NO:36;
(ii) a contiguous portion of SEQ ID NO:36 of at least 20 amino acids;
(iii) the sequence set forth in SEQ ID NO:23;
(iv) a contiguous portion of SEQ ID NO:23 of at least 20 amino acids;
(v) an amino acid sequence that has at least 90%, 91%, 92%, 93%, 94%, 95%, 96%, 97%, 98%, or 99% sequence identity to any of the foregoing.

173. The DNA-targeting system of any of embodiments 142-172, wherein the transcriptional activation domain of ENL comprises the sequence set forth in SEQ ID NO:131 or an amino acid sequence that has at least 90%, 91%, 92%, 93%, 94%, 95%, 96%, 97%, 98%, or 99% sequence identity to any of the foregoing.

174. The DNA-targeting system of any of embodiments 142-173, wherein the transcriptional activation domain of ENL comprises the sequence set forth in SEQ ID NO:131.

175. The DNA-targeting system of any of embodiments 142-174, wherein the transcriptional activation domain of FOXO3 comprises:
(i) the sequence set forth in SEQ ID NO:37;
(ii) a contiguous portion of SEQ ID NO:37 of at least 20 amino acids;
(iii) the sequence set forth in SEQ ID NO:24;
(iv) a contiguous portion of SEQ ID NO:24 of at least 20 amino acids;
(v) an amino acid sequence that has at least 90%, 91%, 92%, 93%, 94%, 95%, 96%, 97%, 98%, or 99% sequence identity to any of the foregoing.

176. The DNA-targeting system of any of embodiments 142-175, wherein the transcriptional activation domain of FOXO3 comprises the sequence set forth in SEQ ID NO:132 or an amino acid sequence that has at least 90%, 91%, 92%, 93%, 94%, 95%, 96%, 97%, 98%, or 99% sequence identity to any of the foregoing.

177. The DNA-targeting system of any of embodiments 142-176, wherein the transcriptional activation domain of FOXO3 comprises the sequence set forth in SEQ ID NO:132.

178. The DNA-targeting system of any of embodiments 142-177, wherein the transcriptional activation domain of PYGO1 comprises:
(i) the sequence set forth in SEQ ID NO:42;
(ii) a contiguous portion of SEQ ID NO:42 of at least 20 amino acids;
(iii) the sequence set forth in SEQ ID NO:29;
(iv) a contiguous portion of SEQ ID NO:29 of at least 20 amino acids;
(v) an amino acid sequence that has at least 90%, 91%, 92%, 93%, 94%, 95%, 96%, 97%, 98%, or 99% sequence identity to any of the foregoing.

179. The DNA-targeting system of any of embodiments 142-178, wherein the transcriptional activation domain of PYGO1 comprises the sequence set forth in SEQ ID NO:130 or an amino acid sequence that has at least 90%, 91%, 92%, 93%, 94%, 95%, 96%, 97%, 98%, or 99% sequence identity to any of the foregoing.

180. The DNA-targeting system of any of embodiments 142-179, wherein the transcriptional activation domain of PYGO1 comprises the sequence set forth in SEQ ID NO:130.

181. The DNA-targeting system of any of embodiments 142-180, wherein the transcriptional activation domain of HSH2D comprises:
(i) the sequence set forth in SEQ ID NO:38;
(ii) a contiguous portion of SEQ ID NO:38 of at least 20 amino acids;
(iii) the sequence set forth in SEQ ID NO:25;
(iv) a contiguous portion of SEQ ID NO:25 of at least 20 amino acids;
(v) an amino acid sequence that has at least 90%, 91%, 92%, 93%, 94%, 95%, 96%, 97%, 98%, or 99% sequence identity to any of the foregoing.

182. The DNA-targeting system of any of embodiments 142-181, wherein the transcriptional activation domain of HSH2D comprises the sequence set forth in SEQ ID NO:134 or an amino acid sequence that has at least 90%, 91%, 92%, 93%, 94%, 95%, 96%, 97%, 98%, or 99% sequence identity to any of the foregoing.

183. The DNA-targeting system of any of embodiments 142-182, wherein the transcriptional activation domain of HSH2D comprises the sequence set forth in SEQ ID NO:134.

184. The DNA-targeting system of any of embodiments 142-183, wherein the transcriptional activation domain of NCOA2 comprises:
(i) the sequence set forth in SEQ ID NO:39;
(ii) a contiguous portion of SEQ ID NO:39 of at least 20 amino acids;
(iii) the sequence set forth in SEQ ID NO:26;
(iv) a contiguous portion of SEQ ID NO:26 of at least 20 amino acids;
(v) an amino acid sequence that has at least 90%, 91%, 92%, 93%, 94%, 95%, 96%, 97%, 98%, or 99% sequence identity to any of the foregoing.

185. The DNA-targeting system of any of embodiments 142-184, wherein the transcriptional activation domain of NCOA2 comprises the sequence set forth in SEQ ID NO:135 or an amino acid sequence that has at least 90%, 91%, 92%, 93%, 94%, 95%, 96%, 97%, 98%, or 99% sequence identity to any of the foregoing.

186. The DNA-targeting system of any of embodiments 142-185, wherein the transcriptional activation domain of NCOA2 comprises the sequence set forth in SEQ ID NO:135.

187. The DNA-targeting system of any of embodiments 142-186 wherein the transcriptional activation domain of NOTCH2 comprises:
(i) the sequence set forth in SEQ ID NO:46;
(ii) a contiguous portion of SEQ ID NO:46 of at least 20 amino acids;
(iii) the sequence set forth in SEQ ID NO:33;
(iv) a contiguous portion of SEQ ID NO:33 of at least 20 amino acids;
(v) an amino acid sequence that has at least 90%, 91%, 92%, 93%, 94%, 95%, 96%, 97%, 98%, or 99% sequence identity to any of the foregoing.

188. The DNA-targeting system of any of embodiments 142-187, wherein the transcriptional activation domain of NOTCH2 comprises the sequence set forth in SEQ ID NO:136 or an amino acid sequence that has at least 90%, 91%, 92%, 93%, 94%, 95%, 96%, 97%, 98%, or 99% sequence identity to any of the foregoing.

189. The DNA-targeting system of any of embodiments 142-188, wherein the transcriptional activation domain of NOTCH2 comprises the sequence set forth in SEQ ID NO:136. 190. The DNA-targeting system of any of embodiments 142-189, wherein the transcriptional activation domain of DPOLA comprises:
(i) the sequence set forth in SEQ ID NO:35;
(ii) a contiguous portion of SEQ ID NO:35 of at least 20 amino acids;
(iii) the sequence set forth in SEQ ID NO:22;
(iv) a contiguous portion of SEQ ID NO:22 of at least 20 amino acids;
(v) an amino acid sequence that has at least 90%, 91%, 92%, 93%, 94%, 95%, 96%, 97%, 98%, or 99% sequence identity to any of the foregoing.

191. The DNA-targeting system of any of embodiments 142-190, wherein the transcriptional activation domain of DPOLA comprises the sequence set forth in SEQ ID NO:176 or an amino acid sequence that has at least 90%, 91%, 92%, 93%, 94%, 95%, 96%, 97%, 98%, or 99% sequence identity to any of the foregoing.

192. The DNA-targeting system of any of embodiments 142-191, wherein the transcriptional activation domain of DPOLA comprises the sequence set forth in SEQ ID NO:176.

193. The DNA-targeting system of any of embodiments 142-192, wherein the transcriptional activation domain of PSA1 comprises:
(i) the sequence set forth in SEQ ID NO:41;
(ii) a contiguous portion of SEQ ID NO:41 of at least 20 amino acids;
(iii) the sequence set forth in SEQ ID NO:28;
(iv) a contiguous portion of SEQ ID NO:28 of at least 20 amino acids;
(v) an amino acid sequence that has at least 90%, 91%, 92%, 93%, 94%, 95%, 96%, 97%, 98%, or 99% sequence identity to any of the foregoing.

194. The DNA-targeting system of any of embodiments 142-193, wherein the transcriptional activation domain of PSA1 comprises the sequence set forth in SEQ ID NO:177 or an amino acid sequence that has at least 90%, 91%, 92%, 93%, 94%, 95%, 96%, 97%, 98%, or 99% sequence identity to any of the foregoing.

195. The DNA-targeting system of any of embodiments 142-194, wherein the transcriptional activation domain of PSA1 comprises the sequence set forth in SEQ ID NO:177.

196. The DNA-targeting system of any of embodiments 142-195, wherein the transcriptional activation domain of RBM39 comprises:
(i) the sequence set forth in SEQ ID NO:43;
(ii) a contiguous portion of SEQ ID NO:43 of at least 20 amino acids;
(iii) the sequence set forth in SEQ ID NO:30;
(iv) a contiguous portion of SEQ ID NO:30 of at least 20 amino acids;
(v) an amino acid sequence that has at least 90%, 91%, 92%, 93%, 94%, 95%, 96%, 97%, 98%, or 99% sequence identity to any of the foregoing.

197. The DNA-targeting system of any of embodiments 142-196, wherein the transcriptional activation domain of RBM39 comprises the sequence set forth in SEQ ID NO:178 or an amino acid sequence that has at least 90%, 91%, 92%, 93%, 94%, 95%, 96%, 97%, 98%, or 99% sequence identity to any of the foregoing.

198. The DNA-targeting system of any of embodiments 142-197, wherein the transcriptional activation domain of RBM39 comprises the sequence set forth in SEQ ID NO:178.

199. The DNA-targeting system of any of embodiments 142-198, wherein the transcriptional activation domain of HERC2 comprises:
(i) the sequence set forth in SEQ ID NO:44;
(ii) a contiguous portion of SEQ ID NO:44 of at least 20 amino acids;
(iii) the sequence set forth in SEQ ID NO:31;
(iv) a contiguous portion of SEQ ID NO:31 of at least 20 amino acids;
(v) an amino acid sequence that has at least 90%, 91%, 92%, 93%, 94%, 95%, 96%, 97%, 98%, or 99% sequence identity to any of the foregoing.

200. The DNA-targeting system of any of embodiments 142-199, wherein the transcriptional activation domain of HERC2 comprises the sequence set forth in SEQ ID NO:179 or an amino acid sequence that has at least 90%, 91%, 92%, 93%, 94%, 95%, 96%, 97%, 98%, or 99% sequence identity to any of the foregoing.

201. The DNA-targeting system of any of embodiments 142-200, wherein the transcriptional activation domain of HERC2 comprises the sequence set forth in SEQ ID NO:179.

202. The DNA-targeting system of any of embodiments 142-201, wherein the transcriptional activator domain is at least at or about 30, 40, 50, 60, or 70 amino acids in length.

203. The DNA-targeting system of any of embodiments 142-202, wherein the transcriptional activator domain is at least at or about 40 amino acids in length.

204. The DNA-targeting system of any of embodiments 142-202, wherein the transcriptional activator domain is at least at or about 50 amino acids in length.

205. The DNA-targeting system of any of embodiments 142-202, wherein the transcriptional activator domain is at least at or about 60 amino acids in length.

206. The DNA-targeting system of any of embodiments 142-202, wherein the transcriptional activator domain is at least at or about 70 amino acids in length.

207. The DNA-targeting system of any of embodiments 142-202, wherein the transcriptional activator domain is at or about 120, 110, 100, 90, 80, 70, 60, 50, or 40 amino acids or less in length.

208. The DNA-targeting system of any of embodiments 142-202 and 207, wherein the transcriptional activator domain is 70 amino acids or less in length.

209. The DNA-targeting system of any of embodiments 142-202 and 207, wherein the transcriptional activator domain is 60 amino acids or less in length.

210. The DNA-targeting system of any of embodiments 142-202 and 207, wherein the transcriptional activator domain is 50 amino acids or less in length.

211. The DNA-targeting system of any of embodiments 142-201, wherein the transcriptional activator domain is between at or about 40 and at or about 120, at or about 40 and at or about 110, at or about 40 and at or about 100, at or about 40 and at or about 90, at or about 40 and at or about 80, at or about 40 and at or about 70, at or about 40 and at or about 60, or at or about 40 and at or about 50 amino acids in length.

212. The DNA-targeting system of any of embodiments 142-211, wherein the fusion protein comprises a multipartite effector comprising at least two of the two or more transcriptional activation domains.

213. The DNA-targeting system embodiment 212, wherein the multipartite effector is composed of two transcriptional activation domains.

214. The DNA-targeting system embodiment 212 or embodiment 213, wherein the multipartite effector is set forth in any of SEQ ID NOS:140-153, a portion thereof, or an amino acid sequence that has at least 90%, 91%, 92%, 93%, 94%, 95%, 96%, 97%, 98%, or 99% sequence identity to any of the foregoing

215. The DNA-targeting system of any of embodiments 142-212, wherein the two or more transcriptional activation domains is two transcriptional activation domains.

216. The DNA-targeting system of any of embodiments 142-215, wherein the fusion protein comprises:
a transcriptional activation domain of PYGO1 and a transcriptional activation domain of NCOA3;
a transcriptional activation domain of NOTCH2 and a transcriptional activation domain of NCOA3;
a transcriptional activation domain of NCOA3 and a transcriptional activation domain of NCOA3;
a transcriptional activation domain of HSH2D and a transcriptional activation domain of NCOA3;
a transcriptional activation domain of FOXO3 and a transcriptional activation domain of NCOA3;
a transcriptional activation domain of NCOA2 and a transcriptional activation domain of NCOA3;
a transcriptional activation domain of ENL and a transcriptional activation domain of NCOA3;
a transcriptional activation domain of PYGO1 and a transcriptional activation domain of FOXO3;
a transcriptional activation domain of NOTCH2 and a transcriptional activation domain of FOXO3;
a transcriptional activation domain of NCOA3 and a transcriptional activation domain of FOXO3;
a transcriptional activation domain of HSH2D and a transcriptional activation domain of FOXO3;
a transcriptional activation domain of FOXO3 and a transcriptional activation domain of FOXO3;
a transcriptional activation domain of NCOA2 and a transcriptional activation domain of FOXO3; or
a transcriptional activation domain of ENL and a transcriptional activation domain of FOXO3.

217. The DNA-targeting system of any of embodiments 142-216, wherein the fusion protein comprises, in N-terminus to C-terminus order:
a transcriptional activation domain of PYGO1, a linker, and a transcriptional activation domain of NCOA3;
a transcriptional activation domain of NOTCH2, a linker, and a transcriptional activation domain of NCOA3;
a transcriptional activation domain of NCOA3, a linker, and a transcriptional activation domain of NCOA3;
a transcriptional activation domain of HSH2D, a linker, and a transcriptional activation domain of NCOA3;
a transcriptional activation domain of FOXO3, a linker, and a transcriptional activation domain of NCOA3;
a transcriptional activation domain of NCOA2, a linker, and a transcriptional activation domain of NCOA3;
a transcriptional activation domain of ENL, a linker, and a transcriptional activation domain of NCOA3;
a transcriptional activation domain of PYGO1, a linker, and a transcriptional activation domain of FOXO3;
a transcriptional activation domain of NOTCH2, a linker, and a transcriptional activation domain of FOXO3;
a transcriptional activation domain of NCOA3, a linker, and a transcriptional activation domain of FOXO3;
a transcriptional activation domain of HSH2D, a linker, and a transcriptional activation domain of FOXO3;
a transcriptional activation domain of FOXO3, a linker, and a transcriptional activation domain of FOXO3;
a transcriptional activation domain of NCOA2, a linker, and a transcriptional activation domain of FOXO3; or
a transcriptional activation domain of ENL, a linker, and a transcriptional activation domain of FOXO3.

218. The DNA-targeting system of any of embodiments 142-217, wherein the fusion protein comprises the sequence set forth in any one of SEQ ID NOS:140-153, or an amino acid sequence that has at least 90%, 91%, 92%, 93%, 94%, 95%, 96%, 97%, 98%, or 99% sequence identity to any of the foregoing.

219. The DNA-targeting system of any of embodiments 142-218, wherein the fusion protein comprises the sequence set forth in SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, or SEQ ID NO:153.

220. The DNA-targeting system of embodiment any of embodiments 142-219, wherein the fusion protein comprises, in N-terminus to C-terminus order:
a dCas, optionally a dCas9, a transcriptional activation domain of PYGO1 and a transcriptional activation domain of NCOA3;
a dCas, optionally a dCas9, a transcriptional activation domain of NOTCH2 and a transcriptional activation domain of NCOA3;
a dCas, optionally a dCas9, a transcriptional activation domain of NCOA3 and a transcriptional activation domain of NCOA3;
a dCas, optionally a dCas9, a transcriptional activation domain of HSH2D and a transcriptional activation domain of NCOA3;
a dCas, optionally a dCas9, a transcriptional activation domain of FOXO3 and a transcriptional activation domain of NCOA3;
a dCas, optionally a dCas9, a transcriptional activation domain of NCOA2 and a transcriptional activation domain of NCOA3;
a dCas, optionally a dCas9, a transcriptional activation domain of ENL and a transcriptional activation domain of NCOA3;
a dCas, optionally a dCas9, a transcriptional activation domain of PYGO1 and a transcriptional activation domain of FOXO3;
a dCas, optionally a dCas9, a transcriptional activation domain of NOTCH2 and a transcriptional activation domain of FOXO3;
a dCas, optionally a dCas9, a transcriptional activation domain of NCOA3 and a transcriptional activation domain of FOXO3;
a dCas, optionally a dCas9, a transcriptional activation domain of HSH2D and a transcriptional activation domain of FOXO3;
a dCas, optionally a dCas9, a transcriptional activation domain of FOXO3 and a transcriptional activation domain of FOXO3;
a dCas, optionally a dCas9, a transcriptional activation domain of NCOA2 and a transcriptional activation domain of FOXO3; or
a dCas, optionally a dCas9, a transcriptional activation domain of ENL and a transcriptional activation domain of FOXO3.

221. The DNA-targeting system of embodiment any of embodiments 142-219, wherein the fusion protein comprises, in N-terminus to C-terminus order: a ZFP, a transcriptional activation domain of PYGO1 and a transcriptional activation domain of NCOA3;
a ZFP, a transcriptional activation domain of NOTCH2 and a transcriptional activation domain of NCOA3;
a ZFP, a transcriptional activation domain of NCOA3 and a transcriptional activation domain of NCOA3;
a ZFP, a transcriptional activation domain of HSH2D and a transcriptional activation domain of NCOA3;
a ZFP, a transcriptional activation domain of FOXO3 and a transcriptional activation domain of NCOA3;
a ZFP, a transcriptional activation domain of NCOA2 and a transcriptional activation domain of NCOA3;
a ZFP, a transcriptional activation domain of ENL and a transcriptional activation domain of NCOA3;
a ZFP, a transcriptional activation domain of PYGO1 and a transcriptional activation domain of FOXO3;
a ZFP, a transcriptional activation domain of NOTCH2 and a transcriptional activation domain of FOXO3;
a ZFP, a transcriptional activation domain of NCOA3 and a transcriptional activation domain of FOXO3;
a ZFP, a transcriptional activation domain of HSH2D and a transcriptional activation domain of FOXO3;
a ZFP, a transcriptional activation domain of FOXO3 and a transcriptional activation domain of FOXO3;
a ZFP, a transcriptional activation domain of NCOA2 and a transcriptional activation domain of FOXO3; or
a ZFP, a transcriptional activation domain of ENL and a transcriptional activation domain of FOXO3.

222. The DNA-targeting system of any of embodiments 142-219, wherein the fusion protein comprises, in N-terminus to C-terminus order:
a transcriptional activation domain of PYGO1, a transcriptional activation domain of NCOA3, and a dCas, optionally a dCas9;
a transcriptional activation domain of NOTCH2, a transcriptional activation domain of NCOA3, and a dCas, optionally a dCas9;
a transcriptional activation domain of NCOA3, a transcriptional activation domain of NCOA3, and a dCas, optionally a dCas9;
a transcriptional activation domain of HSH2D, a transcriptional activation domain of NCOA3, and a dCas, optionally a dCas9;
a transcriptional activation domain of FOXO3, a transcriptional activation domain of NCOA3, and a dCas, optionally a dCas9;
a transcriptional activation domain of NCOA2, a transcriptional activation domain of NCOA3, and a dCas, optionally a dCas9;
a transcriptional activation domain of ENL, a transcriptional activation domain of NCOA3, and a dCas, optionally a dCas9;
a transcriptional activation domain of PYGO1, a transcriptional activation domain of FOXO3, and a dCas, optionally a dCas9;
a transcriptional activation domain of NOTCH2, a transcriptional activation domain of FOXO3, and a dCas, optionally a dCas9;
a transcriptional activation domain of NCOA3, a transcriptional activation domain of FOXO3, and a dCas, optionally a dCas9;
a transcriptional activation domain of HSH2D, a transcriptional activation domain of FOXO3, and a dCas, optionally a dCas9;
a transcriptional activation domain of FOXO3, a transcriptional activation domain of FOXO3, and a dCas, optionally a dCas9;
a transcriptional activation domain of NCOA2, a transcriptional activation domain of FOXO3, and a dCas, optionally a dCas9; or
a transcriptional activation domain of ENL, a transcriptional activation domain of FOXO3, and a dCas, optionally a dCas9.

223. The DNA-targeting system of any of embodiments 142-219, wherein the fusion protein comprises, in N-terminus to C-terminus order:
a transcriptional activation domain of PYGO1, a transcriptional activation domain of NCOA3, and a ZFP;
a transcriptional activation domain of NOTCH2, a transcriptional activation domain of NCOA3, and a ZFP;
a transcriptional activation domain of NCOA3, a transcriptional activation domain of NCOA3, and a ZFP;
a transcriptional activation domain of HSH2D, a transcriptional activation domain of NCOA3, and a ZFP;
a transcriptional activation domain of FOXO3, a transcriptional activation domain of NCOA3, and a ZFP;
a transcriptional activation domain of NCOA2, a transcriptional activation domain of NCOA3, and a ZFP;
a transcriptional activation domain of ENL, a transcriptional activation domain of NCOA3, and a ZFP;
a transcriptional activation domain of PYGO1, a transcriptional activation domain of FOXO3, and a ZFP;
a transcriptional activation domain of NOTCH2, a transcriptional activation domain of FOXO3, and a ZFP;
a transcriptional activation domain of NCOA3, a transcriptional activation domain of FOXO3, and a ZFP;
a transcriptional activation domain of HSH2D, a transcriptional activation domain of FOXO3, and a ZFP;
a transcriptional activation domain of FOXO3, a transcriptional activation domain of FOXO3, and a ZFP;
a transcriptional activation domain of NCOA2, a transcriptional activation domain of FOXO3, and a ZFP; or
a transcriptional activation domain of ENL, a transcriptional activation domain of FOXO3, and a ZFP.

224. The DNA-targeting system of any of embodiments 142-212 and 216-223, wherein the two or more transcriptional activation domains is three transcriptional activation domains.

225. The DNA-targeting system of any of embodiments 142-212 and 224, wherein the fusion protein comprises a multipartite effector comprising at least three transcriptional activation domains.

226. The DNA-targeting system of embodiment 212 and 225, wherein the multipartite effector is composed of two transcriptional activation domains or three transcriptional activation domains.

227. The DNA-targeting system of embodiment 212, 225 and 226, wherein the multipartite effector is set forth in any of SEQ ID NOS:154-160 and 377, a portion thereof, or an amino acid sequence that has at least 90%, 91%, 92%, 93%, 94%, 95%, 96%, 97%, 98%, or 99% sequence identity to any of the foregoing

228. The DNA-targeting system of any of embodiments 142-212 and 216-227, wherein the fusion protein comprises:
a transcriptional activation domain of PYGO1, a transcriptional activation domain of FOXO3, and a transcriptional activation domain of NCOA3;
a transcriptional activation domain of NOTCH2, a transcriptional activation domain of FOXO3, and a transcriptional activation domain of NCOA3;
a transcriptional activation domain of NCOA3, a transcriptional activation domain of FOXO3, and a transcriptional activation domain of NCOA3;
a transcriptional activation domain of HSH2D, a transcriptional activation domain of FOXO3, and a transcriptional activation domain of NCOA3;
a transcriptional activation domain of FOXO3, a transcriptional activation domain of FOXO3, and a transcriptional activation domain of NCOA3;
a transcriptional activation domain of NCOA2, a transcriptional activation domain of FOXO3, and a transcriptional activation domain of NCOA3;
a transcriptional activation domain of ENL, a transcriptional activation domain of FOXO3, and a transcriptional activation domain of NCOA3; or
a transcriptional activation domain of NCOA3, a transcriptional activation domain of FOXO3, and a transcriptional activation domain of FOXO3.

229. The DNA-targeting system of any of embodiments 212 and 225-228, wherein the multipartite effector comprises, in the N-terminal to C-terminal direction, domains from FOXO3, FOXO3, and NCOA3, respectively

230. The DNA-targeting system of embodiment 229, wherein the multipartite effector comprises the sequence set forth in SEQ ID NO:158, or an amino acid sequence that has at least 90%, 91%, 92%, 93%, 94%, 95%, 96%, 97%, 98%, or 99% sequence identity thereto.

231. The DNA-targeting system of any of embodiments 212 and 225-228, wherein the multipartite effector comprises, in the N-terminal to C-terminal direction, domains from NCOA3, FOXO3, and NCOA3, respectively

232. The DNA-targeting system of embodiment 231, wherein the multipartite effector comprises the sequence set forth in SEQ ID NO:156, or an amino acid sequence that has at least 90%, 91%, 92%, 93%, 94%, 95%, 96%, 97%, 98%, or 99% sequence identity thereto

233. The DNA-targeting system of any of embodiments 212 and 225-228, wherein the multipartite effector comprises, in the N-terminal to C-terminal direction, domains from NCOA2, FOXO3, and NCOA3, respectively

234. The DNA-targeting system of embodiment 233, wherein the multipartite effector comprises the sequence set forth in SEQ ID NO:159, or an amino acid sequence that has at least 90%, 91%, 92%, 93%, 94%, 95%, 96%, 97%, 98%, or 99% sequence identity thereto

235. The DNA-targeting system of any of embodiments 212 and 225-228, wherein the multipartite effector comprises, in the N-terminal to C-terminal direction, domains from PYGO1, FOXO3, and NCOA3, respectively

236. The DNA-targeting system of embodiment 235, wherein the multipartite effector comprises the sequence set forth in SEQ ID NO:154, or an amino acid sequence that has at least 90%, 91%, 92%, 93%, 94%, 95%, 96%, 97%, 98%, or 99% sequence identity thereto

237. The DNA-targeting system of any of embodiments 212 and 225-228, wherein the multipartite effector comprises, in the N-terminal to C-terminal direction, domains from NCOA3, FOXO3, and FOXO3, respectively

238. The DNA-targeting system of embodiment 237, wherein the multipartite effector comprises the sequence set forth in SEQ ID NO:377, or an amino acid sequence that has at least 90%, 91%, 92%, 93%, 94%, 95%, 96%, 97%, 98%, or 99% sequence identity thereto.

239. The DNA-targeting system of any one of any of embodiments 142-212 and 216-238, wherein the fusion protein comprises, in N-terminus to C-terminus order:
a transcriptional activation domain of PYGO1, a linker, a transcriptional activation domain of FOXO3, a linker, and a transcriptional activation domain of NCOA3;
a transcriptional activation domain of NOTCH2, a linker, a transcriptional activation domain of FOXO3, a linker, and a transcriptional activation domain of NCOA3;
a transcriptional activation domain of NCOA3, a linker, a transcriptional activation domain of FOXO3, a linker, and a transcriptional activation domain of NCOA3;
a transcriptional activation domain of HSH2D, a linker, a transcriptional activation domain of FOXO3, a linker, and a transcriptional activation domain of NCOA3;
a transcriptional activation domain of FOXO3, a linker, a transcriptional activation domain of FOXO3, a linker, and a transcriptional activation domain of NCOA3;
a transcriptional activation domain of NCOA2, a linker, a transcriptional activation domain of FOXO3, a linker, and a transcriptional activation domain of NCOA3;
a transcriptional activation domain of ENL, a linker, a transcriptional activation domain of FOXO3, a linker, and a transcriptional activation domain of NCOA3; or
a transcriptional activation domain of NCOA3, a linker, a transcriptional activation domain of FOXO3, a linker, and a transcriptional activation domain of FOXO3.

240. The DNA-targeting system of any of embodiments 142-212 and 216-239, wherein the fusion protein comprises the sequence set forth in any one of SEQ ID NOS:154-160 and 377 or an amino acid sequence that has at least 90%, 91%, 92%, 93%, 94%, 95%, 96%, 97%, 98%, or 99% sequence identity to any of the foregoing.

241. The DNA-targeting system of any of embodiments 142-212 and 216-240, wherein the fusion protein comprises the sequence set forth in SEQ ID NO:154, SEQ ID NO:155, SEQ ID NO:156, SEQ ID NO:157, SEQ ID NO:158, SEQ ID NO:159, or SEQ ID NO:160 or SEQ ID NO: 377.

242. The DNA-targeting system of any of embodiments 142-212 and 216-241, wherein the fusion protein comprises a transcriptional activation domain of PYGO1, a linker, a transcriptional activation domain of FOXO3, a linker, and a transcriptional activation domain of NCOA3.

243. The DNA-targeting system of any of embodiments 142-212 and 216-242, wherein the fusion protein comprises the sequence set forth in SEQ ID NO:154.

244. The DNA-targeting system of any of embodiments 142-212 and 216-241, wherein the fusion protein comprises a transcriptional activation domain of NCOA3, a linker, a transcriptional activation domain of FOXO3, a linker, and a transcriptional activation domain of NCOA3.

245. The DNA-targeting system of any of embodiments 142-212 and 216-241, and 244, wherein the fusion protein comprises the sequence set forth in SEQ ID NO:156.

246. The DNA-targeting system of any of embodiments 142-212 and 216-241, wherein the fusion protein comprises a transcriptional activation domain of FOXO3, a linker, a transcriptional activation domain of FOXO3, a linker, and a transcriptional activation domain of NCOA3.

247. The DNA-targeting system of any of embodiments 142-212 and 216-241, and 246, wherein the fusion protein comprises the sequence set forth in SEQ ID NO:158.

248. The DNA-targeting system of any of embodiments 142-212 and 216-241, wherein the fusion protein comprises a transcriptional activation domain of NCOA2, a linker, a transcriptional activation domain of FOXO3, a linker, and a transcriptional activation domain of NCOA3.

249. The DNA-targeting system of any of embodiments 142-212 and 216-241, and 248, wherein the fusion protein comprises the sequence set forth in SEQ ID NO:159.

250. The DNA-targeting system of any of embodiments 142-212 and 216-241, wherein the fusion protein comprises, in N-terminus to C-terminus order:
a Cas9, optionally dCas9, a linker, a transcriptional activation domain of PYGO1, a linker, a transcriptional activation domain of FOXO3, a linker, and a transcriptional activation domain of NCOA3;
a Cas9, optionally dCas9, a linker, a transcriptional activation domain of NOTCH2, a linker, a transcriptional activation domain of FOXO3, a linker, and a transcriptional activation domain of NCOA3;
a Cas9, optionally dCas9, a linker, a transcriptional activation domain of NCOA3, a linker, a transcriptional activation domain of FOXO3, a linker, and a transcriptional activation domain of NCOA3;
a Cas9, optionally dCas9, a linker, a transcriptional activation domain of HSH2D, a linker, a transcriptional activation domain of FOXO3, a linker, and a transcriptional activation domain of NCOA3;
a Cas9, optionally dCas9, a linker, a transcriptional activation domain of FOXO3, a linker, a transcriptional activation domain of FOXO3, a linker, and a transcriptional activation domain of NCOA3;
a Cas9, optionally dCas9, a linker, a transcriptional activation domain of NCOA2, a linker, a transcriptional activation domain of FOXO3, a linker, and a transcriptional activation domain of NCOA3; or
a Cas9, optionally dCas9, a linker, a transcriptional activation domain of ENL, a linker, a transcriptional activation domain of FOXO3, a linker, and a transcriptional activation domain of NCOA3; or
a Cas9, optionally dCas9, a linker, a transcriptional activation domain of NCOA3, a linker, a transcriptional activation domain of FOXO3, a linker, and a transcriptional activation domain of FOXO3.

251. The DNA-targeting system of any of embodiments 142-212 and 216-241, wherein the fusion protein comprises, in N-terminus to C-terminus order:
a ZFP, a linker, a transcriptional activation domain of PYGO1, a linker, a transcriptional activation domain of FOXO3, a linker, and a transcriptional activation domain of NCOA3;
a ZFP, a linker, a transcriptional activation domain of NOTCH2, a linker, a transcriptional activation domain of FOXO3, a linker, and a transcriptional activation domain of NCOA3;
a ZFP, a linker, a transcriptional activation domain of NCOA3, a linker, a transcriptional activation domain of FOXO3, a linker, and a transcriptional activation domain of NCOA3;
a ZFP, a linker, a transcriptional activation domain of HSH2D, a linker, a transcriptional activation domain of FOXO3, a linker, and a transcriptional activation domain of NCOA3;
a ZFP, a linker, a transcriptional activation domain of FOXO3, a linker, a transcriptional activation domain of FOXO3, a linker, and a transcriptional activation domain of NCOA3;
a ZFP, a linker, a transcriptional activation domain of NCOA2, a linker, a transcriptional activation domain of FOXO3, a linker, and a transcriptional activation domain of NCOA3; or
a ZFP, a linker, a transcriptional activation domain of ENL, a linker, a transcriptional activation domain of FOXO3, a linker, and a transcriptional activation domain of NCOA3; or
a ZFP, a linker, a transcriptional activation domain of NCOA3, a linker, a transcriptional activation domain of FOXO3, a linker, and a transcriptional activation domain of FOXO3.

252. The DNA-targeting system of any of embodiments 142-212 and 216-241, wherein the fusion protein comprises, in N-terminus to C-terminus order:

a transcriptional activation domain of PYGO1, a linker, a transcriptional activation domain of FOXO3, a linker, a transcriptional activation domain of NCOA3, a linker, and a Cas9, optionally a dCas9;
a transcriptional activation domain of NOTCH2, a linker, a transcriptional activation domain of FOXO3, a linker, a transcriptional activation domain of NCOA3, a linker, and a Cas9, optionally a dCas9;
a transcriptional activation domain of NCOA3, a linker, a transcriptional activation domain of FOXO3, a linker, a transcriptional activation domain of NCOA3, a linker, and a Cas9, optionally a dCas9;
a transcriptional activation domain of HSH2D, a linker, a transcriptional activation domain of FOXO3, a linker, a transcriptional activation domain of NCOA3, a linker, and a Cas9, optionally a dCas9;
a transcriptional activation domain of FOXO3, a linker, a transcriptional activation domain of FOXO3, a linker, a transcriptional activation domain of NCOA3, a linker, and a Cas9, optionally a dCas9;
a transcriptional activation domain of NCOA2, a linker, a transcriptional activation domain of FOXO3, a linker, a transcriptional activation domain of NCOA3, a linker, and a Cas9, optionally a dCas9;
a transcriptional activation domain of ENL, a linker, a transcriptional activation domain of FOXO3, a linker, a transcriptional activation domain of NCOA3, a linker, and a Cas9, optionally a dCas9; or
a transcriptional activation domain of NCOA3, a linker, a transcriptional activation domain of FOXO3, a linker, a transcriptional activation domain of FOXO3, a linker, and a Cas9, optionally a dCas9.

253. The DNA-targeting system of any of embodiments 142-212 and 216-241, wherein the fusion protein comprises, in N-terminus to C-terminus order:
a transcriptional activation domain of PYGO1, a linker, a transcriptional activation domain of FOXO3, a linker, a transcriptional activation domain of NCOA3, a linker, and a ZFP;
a transcriptional activation domain of NOTCH2, a linker, a transcriptional activation domain of FOXO3, a linker, a transcriptional activation domain of NCOA3, a linker, and a ZFP;
a transcriptional activation domain of NCOA3, a linker, a transcriptional activation domain of FOXO3, a linker, a transcriptional activation domain of NCOA3, a linker, and a ZFP;
a transcriptional activation domain of HSH2D, a linker, a transcriptional activation domain of FOXO3, a linker, a transcriptional activation domain of NCOA3, a linker, and a ZFP;
a transcriptional activation domain of FOXO3, a linker, a transcriptional activation domain of FOXO3, a linker, a transcriptional activation domain of NCOA3, a linker, and a ZFP;
a transcriptional activation domain of NCOA2, a linker, a transcriptional activation domain of FOXO3, a linker, a transcriptional activation domain of NCOA3, a linker, and a ZFP;
a transcriptional activation domain of ENL, a linker, a transcriptional activation domain of FOXO3, a linker, a transcriptional activation domain of NCOA3, a linker, and a ZFP; or
a transcriptional activation domain of NCOA3, a linker, a transcriptional activation domain of FOXO3, a linker, a transcriptional activation domain of FOXO3, a linker, and a ZFP.

254. The DNA-targeting system of any of embodiments 142-212, 216-223, and 228-253, wherein the two or more transcriptional activation domains comprises four transcriptional activation domains.

255. The DNA-targeting system of any of embodiments 142-212, 216-223, and 228-253, wherein the two or more transcriptional activation domains comprises five transcriptional activation domains.

256. The DNA-targeting system of any of embodiments 142-255, further comprising one or more linkers.

257. The DNA-targeting system of any of embodiments 142-256, wherein a linker of one or more linkers is positioned between the two or more transcriptional activation domains and/or positioned between the polypeptide component of the DNA-targeting domain and one of the two or more transcriptional activation domains.

258. The DNA-targeting system of any of embodiments 256 and 257, wherein the linker is a polypeptide linker.

259. The DNA-targeting system of embodiment 258, wherein the polypeptide linker comprises a sequence selected from among SEQ ID NOS:62-67, 96, and 137-139.

260. The DNA-targeting system of any of embodiments 142-259, further comprising one or more nuclear localization signals (NLSs).

261. The DNA-targeting system of embodiment 260, wherein the one or more NLSs comprises two or more NLSs.

262. The DNA-targeting system of embodiment 260 or 261, wherein a NLS of one or more NLSs is positioned between the two or more transcriptional activation domains.

263. The DNA-targeting system of any of embodiments 218-262, wherein a NLS of the one or more NLSs is positioned between the polypeptide component of the DNA-targeting domain and one of the two or more transcriptional activation domains.

264. The DNA-targeting system of any of embodiments 218-263, wherein the one or more NLSs comprises a sequence selected from among SEQ ID NOS:69-84.

265. The DNA-targeting system of any of embodiments 142-264, wherein the fusion protein comprises the sequence set forth in any one of SEQ ID NOS:181-187, or an amino acid sequence that has at least 90%, 91%, 92%, 93%, 94%, 95%, 96%, 97%, 98%, or 99% sequence identity to any of the foregoing.

266. The DNA-targeting system of any of embodiments 142-265, wherein the fusion protein comprises the sequence set forth in SEQ ID NO:181, SEQ ID NO:182, SEQ ID NO:183, SEQ ID NO:184, SEQ ID NO:185, SEQ ID NO:186, or SEQ ID NO:187.

267. The DNA-targeting system of any of embodiments 142-266, further comprising a tag.

268. The DNA-targeting system of embodiment 267, wherein the tag comprises an epitope tag or a split protein tag.

269. The DNA-targeting system of embodiment 267 or 268, wherein the tag is selected from among SEQ ID NOS:61, 88, 92, and 167.

270. The DNA-targeting system of any of embodiments 142-269, wherein the fusion protein comprises the sequence set forth in any one of SEQ ID NOS:272-278, or an amino acid sequence that has at least 90%, 91%, 92%, 93%, 94%, 95%, 96%, 97%, 98%, or 99% sequence identity to any of the foregoing.

271. The DNA-targeting system of any of embodiments 142-270, wherein the fusion protein comprises the sequence set forth in SEQ ID NO:272, SEQ ID NO:273, SEQ ID NO:274, SEQ ID NO:275, SEQ ID NO:276, SEQ ID NO:277, or SEQ ID NO:278.

272. The DNA-targeting system of any of embodiments 142-271, wherein the DNA-targeting domain is capable of hybridizing to the target site or is complementary to the target site at the endogenous locus.

273. The DNA-targeting system of any of embodiments 142-272, wherein the at least one gRNA is capable of hybridizing to the target site or is complementary to the target site at the endogenous locus.

274. The DNA-targeting system of any of embodiments 142-273, wherein the target site is located at a regulatory DNA element of the endogenous locus.

275. The DNA-targeting system of embodiment 274, wherein the regulatory DNA element is selected from among a promoter, an upstream regulatory element, an enhancer, an exon, an intron, a 5′ untranslated region (UTR), a 3′ UTR, or a downstream regulatory element.

276. The DNA-targeting system of any of embodiments 142-275, wherein the endogenous locus is FXN.

277. The DNA-targeting system of any of embodiments 142-276, wherein the target site is located within the genomic coordinates hg38 chr9:69,027,282-69,028,497 or hg38 chr9:69,027,282-69,028,497.

278. The DNA-targeting system of any of embodiments 142-277, wherein the target site is located within the genomic coordinates hg38 chr9:69,027,615-69,028,101.

279. The DNA-targeting system of any of embodiments 142-277, wherein the target site comprises a sequence set forth in any one of SEQ ID NOS:208-228.

280. The DNA-targeting system of any of embodiments 142-277 and 279, wherein the target site comprises a sequence set forth in SEQ ID NO:208.

281. The DNA-targeting system of any of embodiments 142-277 and 279, wherein the target site comprises a sequence set forth in SEQ ID NO:214.

282. The DNA-targeting system of any of embodiments 142-277, wherein the target site comprises a sequence set forth in SEQ ID NO:228.

283. The DNA-targeting system of any of embodiments 141, 143, 145, and 148-277, wherein the gRNA comprises a sequence set forth in any one of SEQ ID NOS:229-249.

284. The DNA-targeting system of any of embodiments 141, 143, 145, 148-277, and 283, wherein the gRNA comprises a sequence set forth in SEQ ID NO:229.

285. The DNA-targeting system of any of embodiments 141, 143, 145, 148-277, and 283, wherein the gRNA comprises a sequence set forth in SEQ ID NO:235.

286. The DNA-targeting system of any of embodiments 141, 143, 145, 148-277, and 283, wherein the gRNA comprises a sequence set forth in SEQ ID NO:249.

287. A polynucleotide comprising a sequence encoding the fusion protein of any of embodiments 1-139 or the DNA-targeting system of any of embodiments 140-286, or a portion or a component of any of the foregoing.

288. The polynucleotide of embodiment 287, wherein the sequence encoding the fusion protein comprises a sequence set forth in any one of SEQ ID NOS:109-122, or a nucleic acid sequence that has at least 90%, 91%, 92%, 93%, 94%, 95%, 96%, 97%, 98%, or 99% sequence identity to any of the foregoing.

289. The polynucleotide of embodiment 287 or 288, wherein the sequence encoding the fusion protein comprises a sequence set forth in any one of SEQ ID NOS:109-122.

290. The polynucleotide of embodiment 287, wherein the sequence encoding the fusion protein comprises a sequence set forth in any one of SEQ ID NOS:123-129, or a nucleic acid sequence that has at least 90%, 91%, 92%, 93%, 94%, 95%, 96%, 97%, 98%, or 99% sequence identity to any of the foregoing.

291. The polynucleotide of embodiment 287 or 290, wherein the sequence encoding the fusion protein comprises a sequence set forth in any one of SEQ ID NOS:123-129.

292. A plurality of polynucleotides, comprising a first polynucleotide comprising the polynucleotide of any of embodiments 287-291, and one or more second polynucleotides encoding an additional portion or an additional component of the fusion protein of any of embodiments 1-120 or the DNA-targeting system of any of embodiments 121-245, or a portion or a component of any of the foregoing.

293. A vector comprising the polynucleotide of any of embodiments 287-291.

294. A vector comprising the plurality of polynucleotides of embodiment 292.

295. The vector of embodiment 293 or 294, wherein the vector is a viral vector.

296. The vector of embodiment 295, wherein the viral vector is an AAV vector.

297. The vector of embodiment 296, wherein the AAV vector is selected from among AAV1, AAV2, AAV3, AAV4, AAV5, AAV6, AAV7, AAV8, AAV9, AAV10, AAV11, AAV12, or AAV-DJ vector, optionally an AAV5 vector or an AAV9 vector.

298. The vector of any of embodiments 295-297, wherein the viral vector is an AAV9 vector.

299. The vector of embodiment 293 or 294, wherein the vector is a non-viral vector selected from: a lipid nanoparticle, a liposome, an exosome, or a cell penetrating peptide.

300. A plurality of vectors, comprising a first vector comprising the vector of any of embodiments 293-299, and one or more second vectors comprising the one or more second polynucleotide of the plurality of polynucleotides of embodiment 251.

301. A cell comprising the fusion protein of any of embodiments 1-139, the DNA-targeting system of any of embodiments 140-286, the polynucleotide of any of embodiments 287-291, the plurality of polynucleotides of embodiment 292, the vector of any of embodiments 293-299, or the plurality of vectors of embodiment 300, or a portion or a component of any of the foregoing.

302. A method for modulating the expression of an endogenous locus in a cell, the method comprising introducing the fusion protein of any of embodiments 1-139, the DNA-targeting system of any of embodiments 140-286, the polynucleotide of any of embodiments 287-291, the plurality of polynucleotides of embodiment 292, the vector of any of embodiments 293-299, or the plurality of vectors of embodiment 300, or a portion or a component of any of the foregoing, into the cell.

303. A method for modulating the expression of an endogenous locus in a subject, the method comprising administering the fusion protein of any of embodiments 1-139, the DNA-targeting system of any of embodiments 140-286, the polynucleotide of any of embodiments 287-291, the plurality of polynucleotides of embodiment 292, the vector of any of embodiments 293-299, or the plurality of vectors of embodiment 300, or a portion or a component of any of the foregoing, to the subject.

304. The method of embodiment 302 or 303, wherein the fusion protein or the DNA-targeting system increases transcription of an endogenous locus when recruited to a target site at the endogenous locus.

305. The method of any of embodiments 302-304, wherein the endogenous locus is FXN.

306. The method of any of embodiments 302-305, wherein the target site is located within the genomic coordinates hg38 chr9:68,940,179-69,205,519 or hg38 chr9:69,027,282-69,028,497.

307. The method of any of embodiments 302-306, wherein the target site is located within the genomic coordinates hg38 chr9:69,027,615-69,028,101.

308. The method of any of embodiments 302-306, wherein the target site comprises a sequence set forth in any one of SEQ ID NOS:208-228.

309. The method of any of embodiments 302-306 and 308, wherein the target site comprises a sequence set forth in SEQ ID NO:208.

310. The method of any of embodiments 302-306 and 308, wherein the target site comprises a sequence set forth in SEQ ID NO:214.

311. The method of any of embodiments 302-308, wherein the target site comprises a sequence set forth in SEQ ID NO:228.

312. The method of any of embodiments 302-311, wherein the cell is from a subject that has or is suspected of having a disease or disorder or the subject has or is suspected of having a disease or disorder.

313. The method of embodiment 312, wherein the disease or disorder is associated with the reduction of expression of the endogenous locus.

314. The method of any of embodiments 302-313, wherein the introducing, contacting or administering is carried out in vivo or ex vivo.

315. The method of any of embodiments 302-314, wherein the subject is a human.

316. A pharmaceutical composition comprising the fusion protein of any of embodiments 1-139, the DNA-targeting system of any of embodiments 140-286, the polynucleotide of any of embodiments 287-291, the plurality of polynucleotides of embodiment 292, the vector of any of embodiments 293-299, or the plurality of vectors of embodiment 300, or a portion or a component of any of the foregoing.

317. The pharmaceutical composition of embodiment 316, for use in treating a disease or disorder.

318. The pharmaceutical composition of embodiment 316, for use in the manufacture of a medicament for treating a disease or disorder.

319. Use of the pharmaceutical composition of embodiment 316 for treating a disease or disorder.

320. Use of the pharmaceutical composition of embodiment 316 in the manufacture of a medicament for treating a disease or disorder.

321. The pharmaceutical composition for use or the use of any of embodiments 316-320, wherein the disease or disorder is associated with the reduction of expression of an endogenous locus.

322. The pharmaceutical composition for use or the use of any of embodiments 316-321, wherein the pharmaceutical composition is to be administered to a subject.

323. The pharmaceutical composition for use or the use of any of embodiments 316-322, wherein the administration is carried out in vivo or ex vivo.

324. The pharmaceutical composition for use or the use of any of embodiments 316-323, wherein the fusion protein or the DNA-targeting system increases transcription of an endogenous locus when recruited to a target site at the endogenous locus.

325. The pharmaceutical composition for use or the use of any of embodiments 316-323, wherein the endogenous locus is frataxin (FXN).

326. The pharmaceutical composition for use or the use of any of embodiments 316-325, wherein the target site is located within the genomic coordinates hg38 chr9:68,940,179-69,205,519 or hg38 chr9:69,027,282-69,028,497. 327. The pharmaceutical composition for use or the use of any of embodiments 316-326, wherein the target site is located within the genomic coordinates hg38 chr9:69,027,615-69,028,101.

328. The pharmaceutical composition for use or the use of any of embodiments 316-326, wherein the target site comprises a sequence set forth in any one of SEQ ID NOS:208-228.

329. The pharmaceutical composition for use or the use of any of embodiments 316-326 and 328, wherein the target site comprises a sequence set forth in SEQ ID NO:208.

330. The pharmaceutical composition for use or the use of any of embodiments 316-326 and 328, wherein the target site comprises a sequence set forth in SEQ ID NO:214.

331. The pharmaceutical composition for use or the use of any of embodiments 316-328, wherein the target site comprises a sequence set forth in SEQ ID NO:228.

332. The pharmaceutical composition for use or the use of any of embodiments 316-331, wherein the subject is a human.

### XIII. Examples

The following examples are included for illustrative purposes only and are not intended to limit the scope of the invention.

### Example 1: Large-Scale Screen for Domains that Act as Transcriptional Activators

A library of plasmids was generated encoding fusion proteins comprising nuclear localized protein fragments, fused to the N-terminus or C-terminus of dCas9. The libraries were screened in a pooled format to identify protein fragments that act as transcriptional activators following targeted recruitment to the promoter of an exemplary target gene.

### A. Transcriptional Activation Domain Screen

A library of plasmids was generated encoding fusion proteins comprising protein fragments of nuclear localized proteins fused to the N-terminus of dSaCas9. A second library was generated with the protein fragments fused to the C-terminus of dSaCas9. The two dSaCas9-protein fragment libraries were each screened separately in a pooled format using induced pluripotent stem cells (iPSCs) expressing an exemplary gRNA targeting an exemplary target gene frataxin (FXN) promoter (SEQ ID NO:175). The gRNA targeted the target site sequence (i.e., comprised the spacer sequence) set forth in SEQ ID NO:208, and further comprised the SaCas9 scaffold sequence set forth in SEQ ID NO: 168.

Nuclear localized protein fragments acting as transcriptional activation domains (i.e., protein fragments containing transcriptional activation domains) were identified as follows. iPSCs expressing the frataxin promoter-targeting gRNA were transduced using lentivirus with the pooled dSaCas9-protein fragment library at a multiplicity of infection (MOI) of 0.4 and incubated for 1 day. Cells were then cultured in the presence of 1 μg/mL puromycin to enrich for transduced cells for 7 days. Cells were then fixed with 4% PFA at room temperature, permeabilized with MeOH overnight at −20° C. and labeled with a mouse monoclonal anti-frataxin fluorophore-conjugated antibody (abcam ab156033). Cells were sorted by flow cytometry into 2 populations comprising the top 10% and bottom 10% of cells based on frataxin protein expression.

Sequencing was performed to identify protein fragments enriched in the frataxin-high populations (transcriptional activators) in comparison to the frataxin-low populations. Genomic DNA was isolated from the sorted populations. Targeted PCR was performed to amplify the protein fragment sequences and append sequencing adapters. Each sample was barcoded separately. Samples were then sequenced using an Illumina MiSeq System. Three replicates of the frataxin-high and frataxin-low population were compared using DEseq2, a method for detecting differentially expressed transcripts.

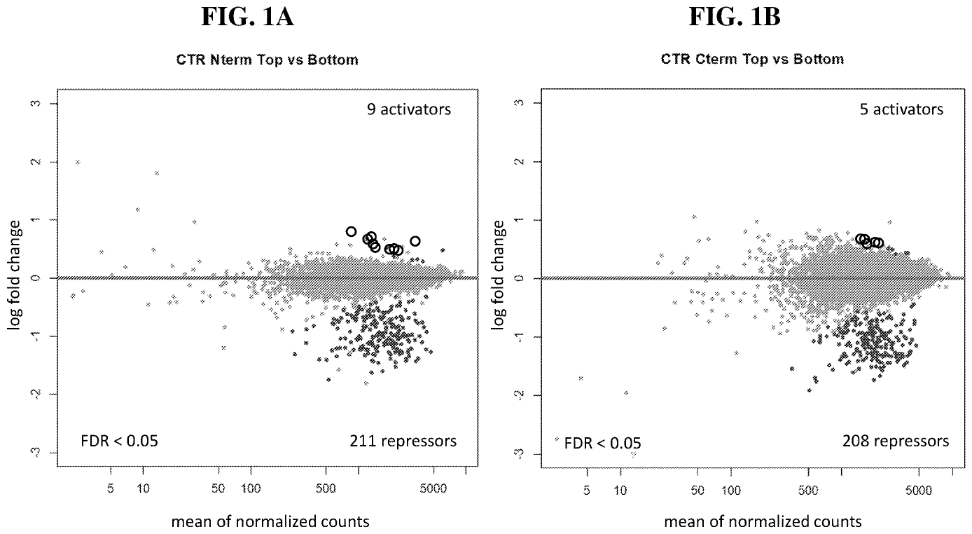

Based on sequencing results, 9 protein fragments acting as transcriptional activators were identified from the N-terminal dSaCas9-protein fragment library (FIG. 1A), and 5 protein fragments acting as transcriptional activators were identified from the C-terminal dSaCas9-protein fragment library (FIG. 1B). 3 of the protein fragments were identified in both screens, including fragments from the proteins NCOA2, NCOA3, and PYGO1.

### B. Validation of Transcriptional Activators

Protein fragments identified as transcriptional activators in the screen described above were verified individually. Wild type iPSCs and iPSCs generated from a subject with a GAA trinucleotide repeat expansion in the frataxin gene (which leads to reduced FXN expression), each expressing the frataxin promoter-targeting gRNA (SEQ ID NO:175), were transduced with nucleic acids encoding dSaCas9-2×VP64 (positive control) or dSaCas9-protein fragment fusion proteins comprising the protein fragments identified as transcriptional activators above. Protein fragments were fused to the N-terminus or C-terminus of dSaCas9.

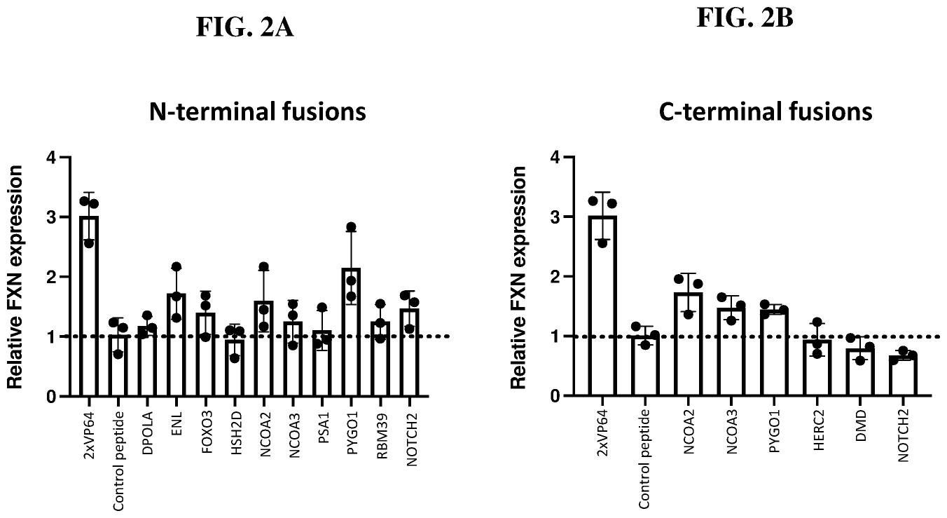

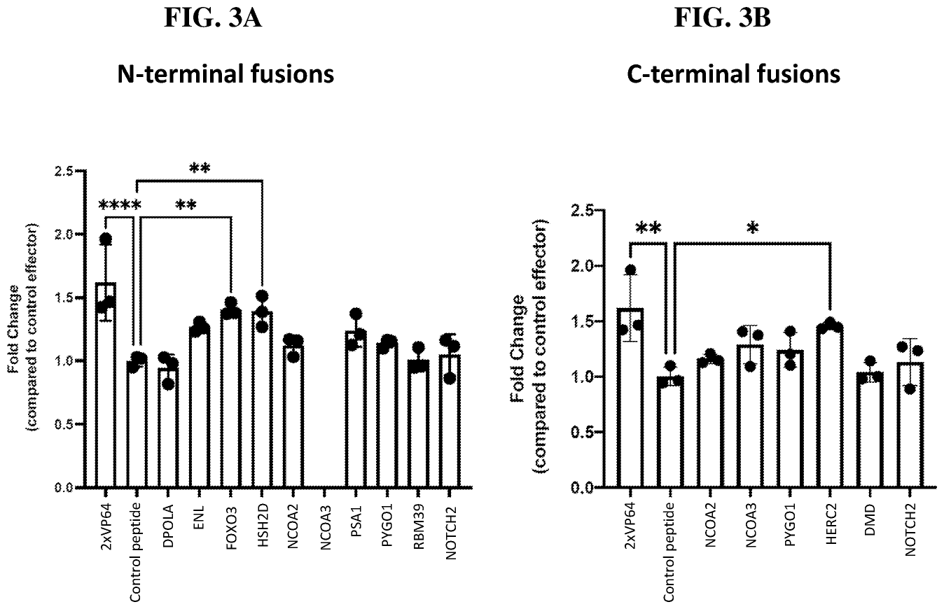

Resulting frataxin mRNA expression was measured by RT-qPCR, in comparison to negative control cells transduced with nucleic acids encoding a dSaCas9-fusion protein with a non-activating control peptide. RT-qPCR was performed using FXN Taqman probes (Hs00175940_m1) and control GAPDH probes (Hs02786624_g1). The results, as shown in FIG. 2A (N-terminal fusions, WT iPSC), FIG. 2B (C-terminal fusions, WT iPSC); FIG. 3A (N-terminal fusions, trinucleotide repeat expansion iPSC) and FIG. 3B (C-terminal fusions, trinucleotide repeat expansion iPSC) showed that several of the protein fragments act as transcriptional activators of the exemplary frataxin mRNA expression when recruited to the frataxin promoter by the dSaCas9 fusion protein and frataxin-targeting gRNA.

The results support the utility of the protein fragments containing transcriptional activation domains identified in the screen for targeted transcriptional activation (e.g., increase in transcription) following recruitment to a target site at an endogenous locus by a DNA-targeting domain.

### Example 2: Design of Multipartite Effectors for Transcriptional Activation

Protein fragments acting as transcriptional activation domains identified in Example 1 were used to design dCas9-effector fusion proteins containing multipartite effectors, for example having two or more of the individual transcriptional activation domains or shorter and/or alternate functional domain or fragments thereof.

Multipartite (e.g., bipartite or tripartite) effectors comprising two or more transcriptional activation domains or fragments thereof, including those from ENL, FOXO3, HSH2D, NCOA2, NCOA3, NOTCH2, and PYGO1, were designed. For several of the nuclear protein fragments, a shorter and/or alternate functional domain fragment (as shown in Table E1) was identified based on a protein domain annotation database. The identified 80 amino-acid protein fragments from the Example 1 above and shorter and/or alternate functional domain fragments are shown in Table E1.

TABLE E1
Transcriptional activation domains

| | 80AA | | Alternate | |
|---|---|---|---|---|
| | protein | 80 AA protein | protein | Alternate protein |
| Source | fragment | fragment | fragment | fragment |
| (gene) | SEQ ID NO: | sequence | SEQ ID NO: | sequence |
| DPOLA | 22 | KSKKGRQEALERLKKAKA | 176 | LERLKKAKAGEKYKYEVED |
| | | GEKYKYEVEDFTGVYEEV | | FTGVYEEVDEEQYSKLVQA |
| | | DEEQYSKLVQARQDDDWI | | RQDDDWIVDDDGIGYVEDG |
| | | VDDDGIGYVEDGREIFDDD | | REIF |
| | | LEDDALD | | |
| ENL | 23 | SKPEKILKKGTYDKAYTDE | 131 | AYTDELVELHRRLMALRER |
| | | LVELHRRLMALRERNVLQ | | NVLQQIVNLIEETGHFNVTN |
| | | QIVNLIEETGHFNVTNTTFD | | TTFDFDLFSLDETTVRKLQS |
| | | FDLFSLDETTVRKLQSCLEA | | CLE |
| | | VAT | | |
| FOXO3 | 24 | DSLSGSSLYSTSANLPVMG | 132 | HEKFPSDLDLDMFNGSLECD |
| | | HEKFPSDLDLDMFNGSLEC | | MESIIRSELMDADGLDFNFD |
| | | DMESIIRSELMDADGLDFNF | | SL |
| | | DSLISTQNVVGLNVGNFTG | | |
| | | AKQ | | |
| HSH2D | 25 | PEWFHGAISREDAENLLES | 134 | WFHGAISREDAENLLESQPL |
| | | QPLGSFLIRVSHSHVGYTLS | | GSFLIRVSHSHVGYTLSYKA |
| | | YKAQSSCCHFMVKLLDDG | | QSSCCHFMVKLLDDGTFMIP |
| | | TFMIPGEKVAHTSLDALVT | | GEKVAHTSLDALVTFH |
| | | FHQQ | | |
| NCOA2 | 26 | PFGSSPDDLLCPHPAAESPS | 135 | ESPSDEGALLDQLYLALRNF |
| | | DEGALLDQLYLALRNFDGL | | DGLEEIDRALGIPELVSQSQA |
| | | EEIDRALGIPELVSQSQAVD | | VDPEQF |
| | | PEQFSSQDSNIMLEQKAPVF | | |
| | | P | | |
| NCOA3 | 27 | LRNSLDDLVGPPSNLEGQS | 133 | EGQSDERALLDQLHTLLSNT |
| | | DERALLDQLHTLLSNTDAT | | DATGLEEIDRALGIPELVNQ |
| | | GLEEIDRALGIPELVNQGQA | | GQALEPKQD |
| | | LEPKQDAFQGQEAAVMMD | | |
| | | QKAG | | |
| PSA1 | 28 | FRNQYDNDVTVWSPQGRIH | 177 | YDNDVTVWSPQGRIHQIEYA |
| | | QIEYAMEAVKQGSATVGL | | ME |
| | | KSKTHAVLVALKRAQSELA | | |
| | | AHQKKILHVDNHIGISIAGL | | |
| | | TADA | | |
| PYGO1 | 29 | RHGHSSSDPVYPCGICTNEV | 130 | PCGICTNEVNDDQDAILCEA |
| | | NDDQDAILCEASCQKWFHR | | SCQKWFHRICTGMTETAYG |
| | | ICTGMTETAYGLLTAEASA | | LLTAEASAVWGCDTCMAD |
| | | VWGCDTCMADKDVQLMR | | |
| | | TRETF | | |
| RBM39 | 30 | GPMRLYVGSLHFNITEDML | 178 | LYVGSLHFNITEDMLRGIFEP |
| | | RGIFEPFGRIESIQLMMDSET | | FGRIESIQLMMDSETGRSKG |
| | | GRSKGYGFITFSDSECAKK | | YGFITFSDSECAKKALEQLN |
| | | ALEQLNGFELAGRPMKVG | | GFELAGRPM |
| | | HVT | | |
| HERC2 | 31 | TLIRKADLENHNKDGGFWT | 179 | RKADLENHNKDGGFWTVID |
| | | VIDGKVYDIKDFQTQSLTG | | GKVYDIKDFQTQSLTGNSIL |
| | | NSILAQFAGEDPVVALEAA | | AQFAGEDPVVALEAALQFE |
| | | LQFEDTRESMHAFCVGQYL | | DTRESMHAFCVGQY |
| | | EPDQ | | |
| NOTCH2 | 33 | NRYSCVCSPGFTGQRCNIDI | 136 | DIDECASNPCRKGATCINGV |
| | | DECASNPCRKGATCINGVN | | NGFRCICPEGPHHPSCY |
| | | GFRCICPEGPHHPSCYSQVN | | |
| | | ECLSNPCIHGNCTGGLSGY | | |
| | | KC | | |

Each bipartite effector included 2 transcriptional activation domains, and each tripartite effector included 3 transcriptional activation domains, each independently selected from among ENL, FOX03, HSH2D, NCOA2, NCOA3, NOTCH2, and PYGO1. Any two of the transcriptional activation domains in a multipartite effector can include the same domain or different domains. Bipartite and tripartite effectors also included one or more peptide linkers, such as a linker comprising a sequence selected from any one of SEQ ID NOS: 137-139. Linkers were located between the transcriptional activation domains and/or to the N- or C-terminus of the activation domains.

Exemplary bipartite and tripartite effectors are shown in Table E2.

TABLE E2
Exemplary Multipartite Effectors

| Bipartite/ | Activator | Exemplary amino |
|---|---|---|
| Tripartite | Domains | acid SEQ ID NO: |
| Bipartite | PYGO1, NCOA3 | 140 |
| Bipartite | NOTCH2, NCOA3 | 141 |
| Bipartite | NCOA3, NCOA3 | 142 |
| Bipartite | HSH2D, NCOA3 | 143 |
| Bipartite | FOXO3, NCOA3 | 144 |
| Bipartite | NCOA2, NCOA3 | 145 |
| Bipartite | ENL, NCOA3 | 146 |
| Bipartite | PYGO1, FOXO3 | 147 |
| Bipartite | NOTCH2, FOXO3 | 148 |
| Bipartite | NCOA3, FOXO3 | 149 |
| Bipartite | HSH2D, FOXO3 | 150 |
| Bipartite | FOXO3, FOXO3 | 151 |
| Bipartite | NCOA2, FOXO3 | 152 |
| Bipartite | ENL, FOXO3 | 153 |
| Tripartite | PYGO1, FOXO3, NCOA3 | 154 |
| Tripartite | NOTCH2, FOXO3, NCOA3 | 155 |
| Tripartite | NCOA3, FOXO3, NCOA3 | 156 |
| Tripartite | HSH2D, FOXO3, NCOA3 | 157 |
| Tripartite | FOXO3, FOXO3, NCOA3 | 158 |
| Tripartite | NCOA2, FOXO3, NCOA3 | 159 |
| Tripartite | ENL, FOXO3, NCOA3 | 160 |

### Example 3: Targeted Transcriptional Activation with Multipartite Effectors for Transcriptional Activation

Exemplary multipartite effectors were assessed for targeted transcriptional activation of an exemplary target gene.

Lentiviral vectors were designed and cloned, each comprising nucleic acids encoding a fusion protein comprising dSaCas9 and a multipartite effector (for example, as described in Example 2) or 2×VP64 (positive control), and a puromycin resistance cassette. iPSCs stably expressing a frataxin promoter-targeting gRNA, and containing a GAA trinucleotide repeat expansion in the frataxin gene (which leads to reduced FXN expression) were transduced with the lentiviral vectors. Cells were selected with 1 μg/mL puromycin for 6 days. On day 7 cells were harvested and RT-qPCR was performed to measure FXN expression, as compared to negative control cells transduced with lentivirus containing the puromycin resistance cassette but no multipartite effector. RT-qPCR was performed using FXN Taqman probes (Hs00175940_m1) and control GAPDH probes (Hs02786624_g1).

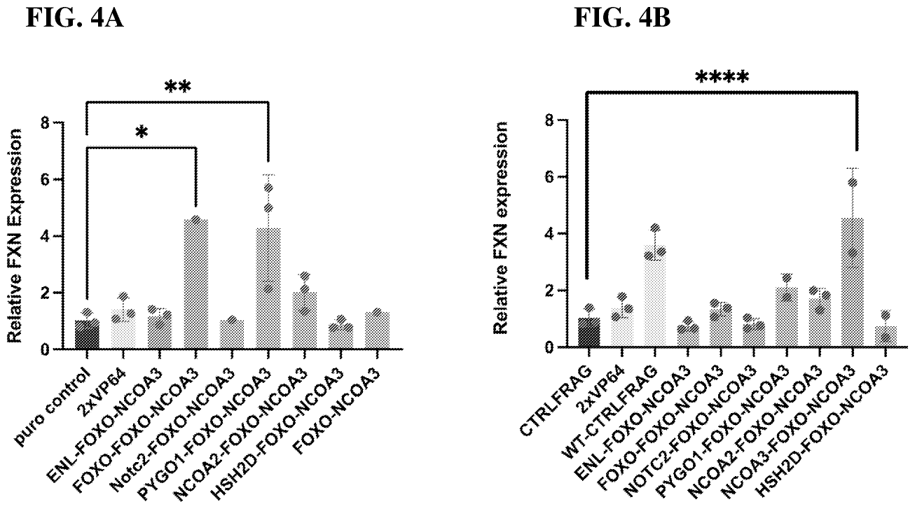

As shown in FIG. 4A, cells transduced with nucleic acids encoding dSaCas9 fusion proteins with multipartite effectors (comprising sequence set forth in any one of SEQ ID NOS: 180-187) exhibited substantially increased expression of FXN in comparison to negative control cells. In addition, several of the multipartite effectors mediated greater activation of FXN than 2×VP64.

All-in-one lentiviral vectors for expression of a FXN promoter-targeting gRNA, a dSaCas9-multipartite effector fusion protein, and EGFP (as a marker for transduction), were designed and cloned. The vectors were transduced into human iPSCs containing a GAA trinucleotide repeat in the frataxin gene (which leads to reduced FXN expression). On day 7, EGFP+ cells were collected and RT-qPCR was performed to measure FXN expression, as compared to negative control cells transduced using a dSaCas9 fusion with a control protein fragment (“CTRLFRAG”) containing a random 80AA sequence that was previously found to not activate or repress FXN. RT-qPCR was performed using FXN Taqman probes (Hs00175940_m1) and control GAPDH probes (Hs02786624_g1).

As shown in FIG. 4B, cells transduced with nucleic acids encoding dSaCas9 fusion proteins with multipartite effectors and a FXN promoter-targeting gRNA exhibited substantially increased expression of FXN in comparison to negative control cells. In addition, several of the multipartite effectors mediated greater activation of FXN than 2×VP64.

The results support the utility of the multipartite effectors for targeted transcriptional activation, including for therapies that may require targeted activation of one or more particular locus, for example in cases where reduced or absent expression of the locus is associated with a disease or disorder. Furthermore, the multipartite effectors can mediate improved targeted transcriptional activation in comparison to other known activation domains, such as VP64.

### Example 4: Targeted Nrf2 Activation in Mouse Neuroblasts

Targeted transcriptional activation of a different exemplary target gene Nrf2 was assessed in a different cell type, mouse neuroblasts using dSaCas9-2×VP64.

Mouse N2a cells (e.g., ATCC Neuro2a CCL-131 mouse neuroblasts) were obtained and cultured. N2a cells were transfected with dSaCas9-2×VP64 and one or all of three exemplary gRNAs targeting Nrf2 (Nuclear factor erythroid 2-related factor 2), as shown in Table E3.

TABLE E3
mouse Nrf2-targeting gRNAs

| | | SEQ |
|---|---|---|
| | | ID |
| gRNA name | gRNA spacer | NO: |
| mouse Nrf2 gRNA A | GCCCGAGGGCGGGGCATGGAC | 190 |
| mouse Nrf2 gRNA B | GCGAGAGGAGGATCAACAGTG | 191 |
| mouse Nrf2 gRNA C | GGCAGTTGGCCTCTTGCAAAGT | 192 |

The three Nrf2-targeting gRNAs were transfected individually or all together in combination. Cells were collected 7 days post-transfection and analyzed by RT-qPCR for expression of the targeted Nrf2 gene in comparison to control cells with no gRNA. RT-qPCR was performed using Taqman Nrf2 probes (Mm00477784_m1) and control GAPDH probes (Mm99999915_g1).

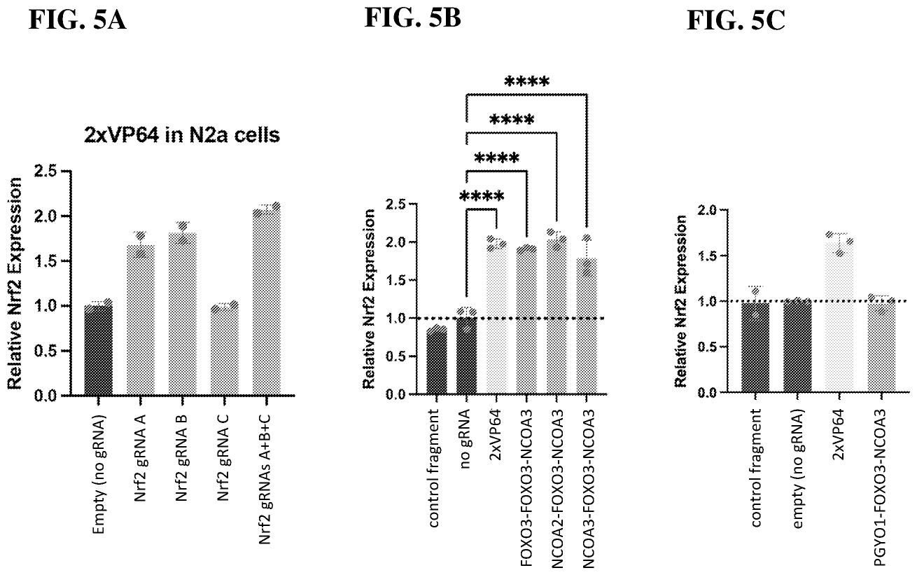

As shown in FIG. 5A, cells transduced with nucleic acids encoding dSaCas9-2×VP64 and the Nrf2-targeting gRNAs exhibited increased expression of Nrf2 in comparison to control cells. Cells transduced with a combination of gRNAs exhibited further increased expression of Nrf2.

The results further support the utility of targeted transcriptional activation using dCas-effector fusion proteins, including in various cell types, target loci and species, and with combinations of multiple gRNAs targeted to a single gene.

### Example 5: Targeted Transcriptional Activation with Multipartite Transcriptional Activators in Mouse Neuroblasts

Multipartite effectors were assessed for targeted transcriptional activation of an exemplary gene in a mouse neuroblast cell line.

Mouse N2a cells (as described above) were transfected with “all-in-one” vectors, each comprising nucleic acids encoding a mouse Nrf2-targeting gRNA (e.g., as described in Example 4) and a fusion protein comprising dSaCas9 and a multipartite effector (e.g., as described in Examples 2 and 3), or 2×VP64 (positive control). In negative control cells, either the vector encoded a fusion protein comprising dSaCas9 and a non-activating control fragment, or the gRNA was omitted. Cells were collected 3 days post-transfection and analyzed by RT-qPCR for Nrf2 expression. RT-qPCR was performed using Taqman Nrf2 probes (Mm00477784_m1) and control GAPDH probes (Mm99999915_g1).

As shown in FIG. 5B, the multipartite effectors FOXO3-FOXO3-NCOA3, NCOA2-FOXO3-NCOA3, and NCOA3-FOXO3-NCOA3 each led to robust activation of Nrf2 expression. FIG. 5C shows that PYGO1-FOXO3-NCOA3 did not lead to substantial activation of Nrf2 expression. The results further support the utility of targeted transcriptional activation using exemplary dCas-multipartite effector fusion proteins, including in various target loci, cell types and species.

The results support the utility of the designed multipartite effectors for targeted transcriptional activation various target loci, across multiple cell types and species, including in neural cells. The results also support the use of all-in-one vectors for delivery of DNA-targeting systems comprising the multipartite effectors. Finally, the results support the utility of the designed multipartite effectors for therapies that may require targeted activation of one or more particular locus, for example, in cases where reduced or absent expression of the locus is associated with a disease or disorder.

### Example 6: Targeted Transcriptional Activation with Multipartite Effectors in Non-Human Primate Fibroblasts

Multipartite effectors are assessed for targeted transcriptional activation in non-human primate cell lines.

Non-human primate cell lines, including those shown in Table E4, are obtained and cultured for the following experiments.

TABLE E4
Non-human primate cell lines

| Cell line Source/ | | | GAPDH Taqman | FXN Taqman |
|---|---|---|---|---|
| Cat. # | Species | Cell type | qPCR Probes | qPCR Probes |
| Coriell Institute, | Macaca Mulatta | Fibroblast | Rh02621745_g1 | Rh02827119_g1 |
| Cat#: AG07107 | (Rhesus monkey) | | | |
| Coriell Institute, | Macaca Fascicularis | Fibroblast | Mf04392546_g1 | Mf04363465_g1 |
| Cat#: AG21329 | (Crab-eating monkey) | | | |

Vectors for expression in non-human primate cell lines are designed, each comprising nucleic acids encoding a fusion protein comprising dCas9 and either a multipartite effector (e.g., as described in Examples 2 and 3) or 2×VP64 (positive control), and a transduction marker (e.g., a puromycin resistance cassette, blasticidin resistance cassette, or EGFP). The lentiviral vectors are transduced into the non-human primate cell lines, which express or are co-transduced with a gRNA targeting the promoter of an exemplary target gene (e.g., FXN), such as any of the gRNAs disclosed herein that can also target conserved sequences of the non-human primate FXN locus.

Following transduction, cells are enriched for transduced cells (e.g. using puromycin selection). Cells are collected 7 days post-transduction and analyzed by RT-qPCR for expression of the target FXN gene. Exemplary RT-qPCR Taqman probes for FXN expression (and control GAPDH expression) are shown in Table E4 for non-human primate cell lines. FXN expression is compared to expression in negative control cells, such as cells transduced with a puromycin resistance cassette alone.

Increased transcription of an exemplary target locus in comparison to negative control cells supports the utility of the multipartite effectors for targeted transcriptional activation across different species and cell types.

### Example 7: AAV Delivery of dCas9-Multipartite Effector Fusions and 2RNAs for Increasing FXN Expression in Cardiomyocytes and Neurons

dCas9 fusion proteins comprising multipartite effectors for transcriptional activation of an exemplary target gene (e.g., FXN) were tested in differentiated cells derived from FA-iPSCs. FA-iPSC-derived cells were transduced with an AAV vector encoding a dCas9-multipartite effector fusion and a FXN targeting gRNA and assessed for increased FXN protein levels.

A. Identification of Multipartite Transcriptional Effectors that Facilitate Increased FXN Protein Levels in FA-Derived Cardiomyocytes

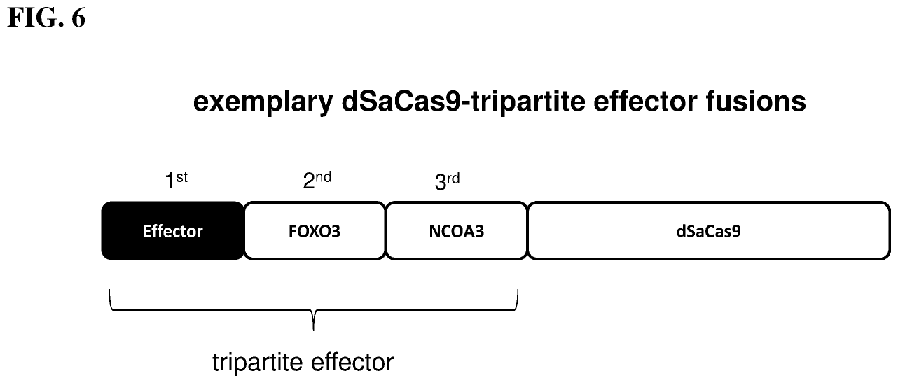

Multipartite effector domains comprising three transcriptional activation domains (i.e. tripartite effectors) designed as described in Example 2 were used to generate a series of dSaCas9-tripartite effector fusions. Each dSaCas9-tripartite effector fusion included in the N- to C-terminus direction, i) the tripartite effector, containing a first transcriptional activation domain that contained one of a PYGO1 domain (e.g. SEQ ID NO: 130), a FOXO3 domain (e.g. SEQ ID NO: 132), an NCOA2 domain (e.g. SEQ ID NO:135) and a NCOA3 (e.g. SEQ ID NO: 133), a second transcriptional activation domain containing a FOXO3 domain (e.g. as set forth in SEQ ID NO:132), a the third transcriptional activation domain comprising an NCOA3 domain (e.g. as set forth in SEQ ID NO:133), and ii) a dSaCas9 protein, generally as shown in FIG. 6. The multipartite effectors comprised domains from PYGO1, FOXO3, and NCOA3, respectively (i.e. PYGO1-FOXO3-NCOA3; e.g. as set forth in SEQ ID NO:154), NCOA3, FOXO3, and NCOA3, respectively (i.e. NCOA3-FOXO3-NCOA3; e.g. as set forth in SEQ ID NO:156), FOXO3, FOXO3, and NCOA3, respectively (i.e. FOXO3-FOXO3-NCOA3; e.g. as set forth in SEQ ID NO:158), or NCOA2, FOXO3, NCOA3, respectively (i.e. NCOA2-FOXO3-NCOA3; e.g. as set forth in SEQ ID NO:159). The individual effector domains were separated by linker sequences, such as a GGGGS linker (e.g. SEQ ID NO:138). Additionally, effectors composed of 1, 2, or 3 tandem copies of NCOA3 (i.e. 1×NCOA3, 2×NCOA3, and 3×NCOA3, respectively) also were tested in the fusion proteins.

iPSCs generated from Friedreich's ataxia patients iPSCs (FA-iPSCs) containing expanded GAA trinucleotide repeats (867/867 repeats) in the frataxin gene were generated and transduced with the dCas9-multipartite effector fusion and a FXN targeting gRNA. FA-iPSC-derived cardiomyocytes were transduced with an AAVDJ vector encoding an exemplary FXN promoter-targeting guide RNA or a non-targeting (NT) guide RNA and one of the dSaCas9 -tripartite effector fusions or dSaCas9 fusions with 1, 2, or 3 tandem copies of NCOA3, at a MOI of 3×10⁴ or 3×10⁵. In this experiment, the gRNA was the gRNA designated gRNA G (SEQ ID NO:256, protospacer set forth in SEQ ID NO:235; targeting SEQ ID NO:214) The effect of dCas9-multipartite effectors on FXN protein levels was compared to a reference dSaCas9-2×VP64 fusion protein in the presence of the same gRNA. Wild-type-derived (WT) and FA-iPSC-derived cardiomyocyte cells not transduced with the full DNA-targeting system were also included as positive and negative controls, respectively. Cells were harvested on day 7 post-transduction to assess FXN protein levels and vector copy number (VCN). Frataxin protein was measured by a Human Frataxin ELISA Kit (abcam #ab176112) and normalized to total protein as assessed by Bicinchoninic acid (BCA) assay.

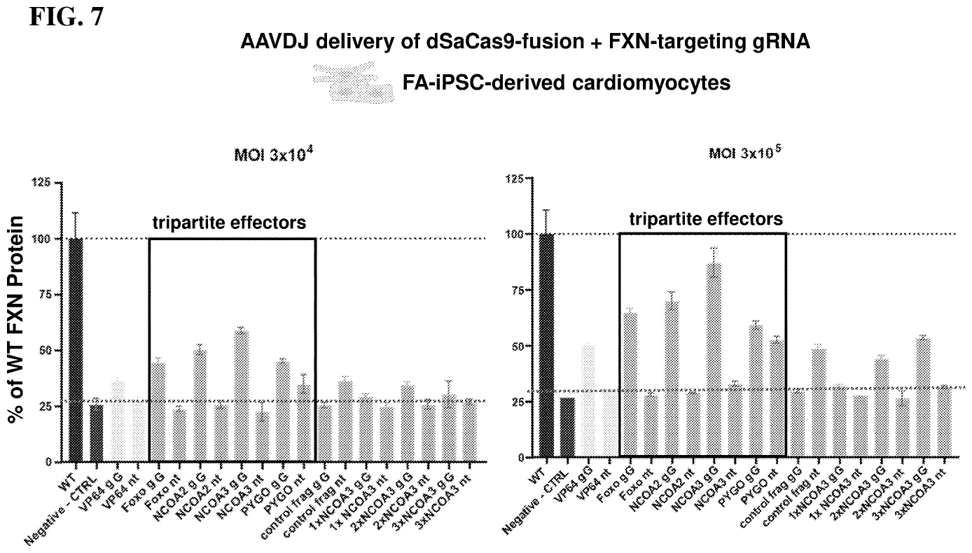

As shown in FIG. 7, there was a robust increase in FXN protein levels at both MOIs in FA-iPSC-derived cardiomyocytes expressing an exemplary promoter-targeting gRNA (e.g. guide RNA G) and several of the dSaCas9-tripartite effector fusions, including for NCOA3-FOXO3-NCOA3, NCOA2-FOXO3-NCOA3, and FOXO3-FOXO3-NCOA3 tripartite effectors. These tripartite effector fusions outperformed dSaCas9-2×VP64 in both MOI conditions, with the NCOA3-FOXO3-NCOA3 tripartite effector fusion exhibiting the strongest increase in FXN protein levels.

At an MOI of 3×10\hat{ }4, FA-iPSC-derived cardiomyocytes expressing guide RNA G and the NCOA3-FOXO3-NCOA3-daSaCas9 tripartite effector fusion increased FXN protein levels to approximately 60% of WT FXN protein levels whereas FA-cardiomyocytes expressing guide RNA G and dSaCas9-2×VP64 increased FXN protein levels to approximately 30% of WT FXN protein levels. A combination of guide RNA G and the NCOA2-FOXO3-NCOA3-daSaCas9 tripartite effector fusion increased FXN protein levels to approximately 50% of WT FXN protein levels and a combination of guide RNA G and the FOXO3-FOXO3-NCOA3-daSaCas9 tripartite effector fusion increased FXN protein levels to approximately 40% of WT FXN protein levels.

At 3×10≡MOI, the NCOA3-FOXO3-NCOA3-daSaCas9 tripartite effector fusion further increased FXN protein levels to almost 90% of WT when paired with guide RNA G. The NCOA2-FOXO3-NCOA3-daSaCas9 tripartite effector fusion increased FXN protein levels to approximately 70% of WT, and the FOXO3-FOXO3-NCOA3-daSaCas9 tripartite effector fusion increased FXN protein levels to approximately 65% of WT. In contrast, dSaCas9-2×VP64 increased FXN protein levels to 50% of WT FXN protein levels under similar conditions.

dSaCas9 effector fusions with activation domains composed solely of 1, 2, or 3 copies of NCOA3 did not substantially increase FXN protein levels in FA-iPSC-derived cardiomyocytes expressing guide RNA G at 3×10\hat{ }4 MOI. However, at 3×10\hat{ }5 MOI, dSaCas9 effector fusions with activation domains comprising 2 or 3 copies of NCOA3 increased FXN protein levels to 40% and 50% of WT in FA-iPSC-derived cardiomyocytes expressing guide RNA G.

B. The Tripartite NCOA3 Effector Increased FXN Levels More Effectively than 2×VP64.

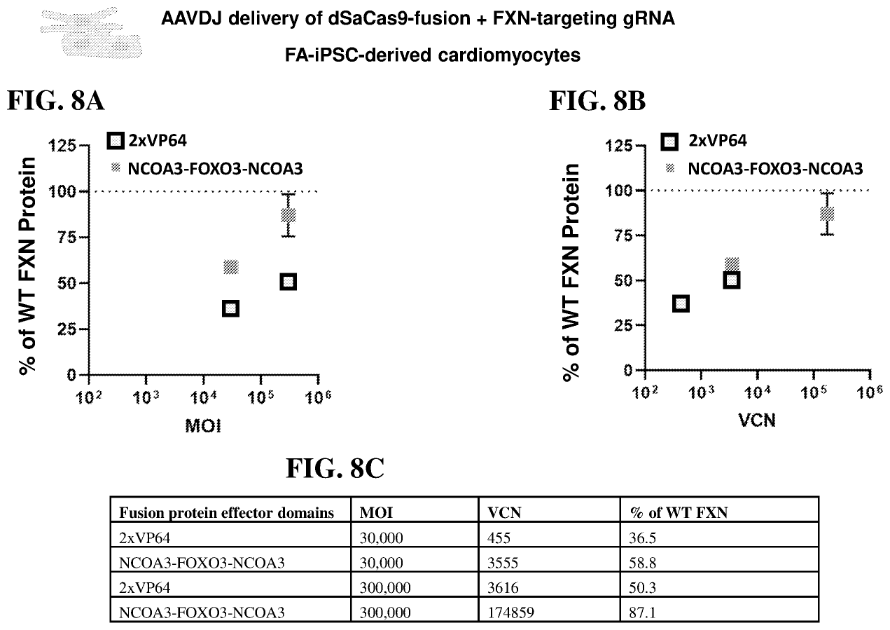

The VCN was calculated for FA-iPSC-derived cardiomyocytes expressing gRNA G and either the NCOA3-FOXO3-NCOA3-daSaCas9 or dSaCas9-2×VP64 effector fusion at various MOIs. As shown in FIG. 8A, NCOA3-FOXO3-NCOA3-daSaCas9 exhibited a higher VCN than dSaCas9-2×VP64 at both MOIs tested. As shown in FIG. 8B, NCOA3-FOXO3-NCOA3 still outperformed 2×VP64 at similar VCNs. Furthermore, NCOA3-FOXO3-NCOA3-daSaCas9 reached a similar VCN as dSaCas9-2×VP64 at a 10-fold lower MOI (FIG. 8C). The numeric values for the data represented in FIG. 8A and FIG. 8B are shown in FIG. 8C and Table E5.

TABLE E5
Transduction efficiency and resulting FXN expression
in FA-iPSC-derived cardiomyocytes transduced with gRNA
G and NCOA3-FOXO3-NCOA3-daSaCas9 or dSaCas9-2xVP64

| Fusion protein | | | |
|---|---|---|---|
| effector domains | MOI | VCN | % of WT FXN |
| 2xVP64 | 30,000 | 455 | 36.5 |
| NCOA3-FOXO3-NCOA3 | 30,000 | 3555 | 58.8 |
| 2xVP64 | 300,000 | 3616 | 50.3 |
| NCOA3-FOXO3-NCOA3 | 300,000 | 174859 | 87.1 |

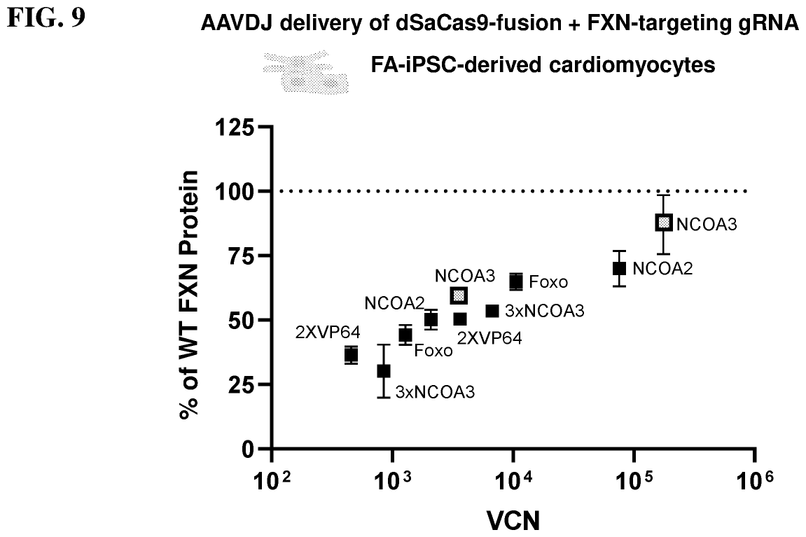

Further, the NCOA3-FOXO3-NCOA3-daSaCas9 tripartite fusion outperformed other tripartite effector fusions and dSaCas9-2×VP64 when adjusted for VCN (FIG. 9).

C. AAV Delivery of dSaCas9-Tripartite Effector Fusions Increases FXN Protein and mRNA Levels in FA-Derived Cells

Next, AAV delivery of the dSaCas9-tripartite effector fusions was assessed for the capacity to increase FXN protein levels in FA-iPSC-derived cardiomyocytes harboring expanded GAA trinucleotide repeats (867/867). FA-iPSC-derived cardiomyocytes were transduced with AAVDJ vectors encoding gRNA G or a non-targeting gRNA (NT) and one of the following dSaCas9-tripartite effector fusions: FOXO3-FOXO3-NCOA3-dSaCas9, NCOA2-FOXO3-NCOA3-dSaCas9, or NCOA3-FOXO3-NCOA3-dSaCas9. dSaCas9-2×VP64 was included as a control and a GFP control construct was used to transduce healthy (CTR GFP) and FA-derived cardiomyocytes (GFP). Cells were harvested on day 7 post-transduction to assess FXN protein levels by ELISA, generally as described above.

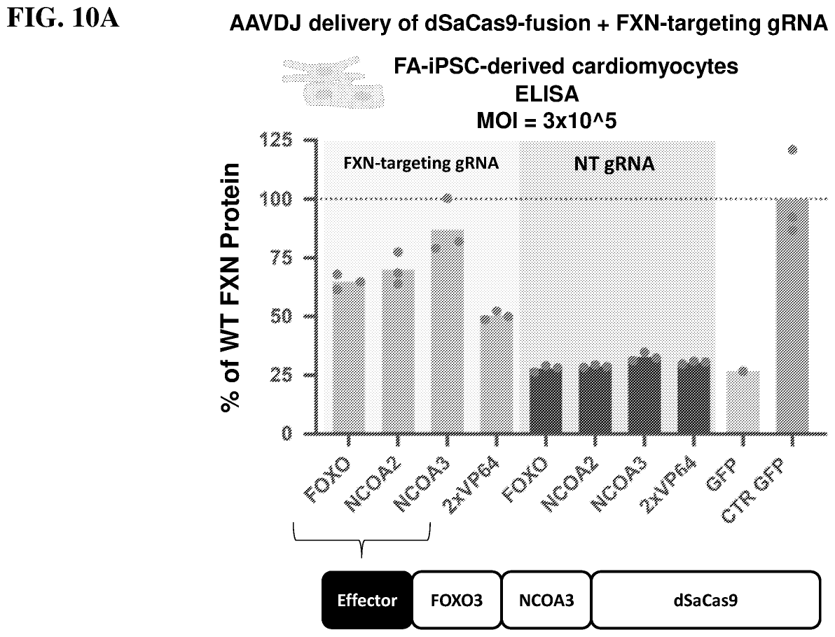

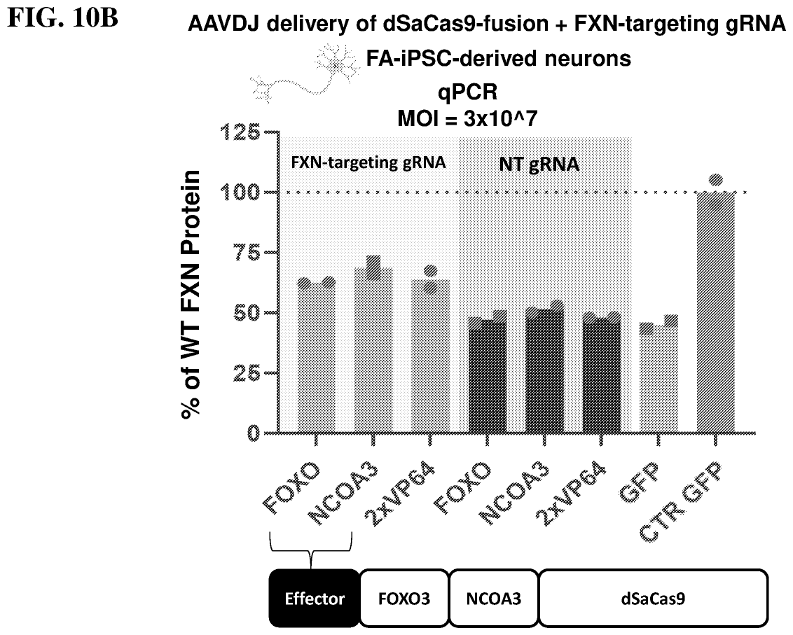

As shown in FIG. 10A, all dSaCas9 fusions with all three of the tripartite effectors increased FXN protein levels to a greater extent than dSaCas9-2×VP64 in FA-iPSC-derived cardiomyocytes expressing gRNA G. Expression of gRNA G and NCOA3-FOXO3-NCOA3-dSaCas9 increased FXN protein levels to approximately 80% of WT levels in FA-iPSC-derived cardiomyocytes. In comparison, the dSaCas9-2×VP64 increased FXN protein levels to approximately 50% of WT, while the negative GFP-transduced and non-targeting (NT) gRNA controls all remained at approximately 25% of WT FXN levels.

AAV delivery of the tripartite effector fusions was assessed for the capacity to increase FXN expression in FA-iPSC-derived neurons. FA-iPSC-derived neurons were transduced with AAVDJ vectors encoding either gRNA G or the non-targeting (NT) gRNA and FOXO3-FOXO3-NCOA3-dSaCas9, NCOA3-FOXO3-NCOA3-dSaCas9 or dSaCas9-2×VP64. Healthy (CTR GFP) and FA-derived (GFP) neurons were transduced with a GFP construct as positive and negative controls. Cells were harvested on day 7 post-transduction to assess FXN mRNA expression levels by RT-qPCR (normalized to GAPDH) and analyzed as fold change in comparison to FXN mRNA expression levels in WT control cells. As shown in FIG. 10B, each of the effector fusion proteins increased FXN mRNA levels in cells expressing gRNA G, in comparison to the NT gRNA control. The FOXO3-FOXO3-NCOA3-dSaCas9 and NCOA3-FOXO3-NCOA3-dSaCas9 tripartite effector fusions increased FXN mRNA levels to a similar or greater extent as dSaCas9-2×VP64 in FA-iPSC-derived neurons expressing gRNA G.

In summary, these results demonstrated that the tripartite effectors described herein function as potent activators of gene expression, as evidenced by activation leading to increased expression of the exemplary FXN gene when targeted to a FXN locus, and with activity similar to or exceeding that of 2×VP64. Further, dSaCas9-tripartite effector fusions paired with a gene-targeting gRNA (e.g. FXN targeting gRNA) were shown to increase both FXN protein and mRNA levels in FA-iPSC-derived cardiomyocytes and neurons. The tripartite effectors were derived from human proteins, in comparison to the virus-derived VP64 effectors. The results support the therapeutic utility of the tripartite effectors for targeted activation of gene expression, including in fusion proteins capable of targeting or being targeted to a particular locus in which gene expression is desired to be increased.

### Example 8: Fusion Proteins Containing eZFPs and Tripartite Effector Domains for Targeted Activation of FXN Expression

Exemplary tripartite effectors were fused with an eZFP targeting a promoter or enhancer region of an exemplary target gene (e.g., FXN) and tested for transcriptional activation. FA-derived cells were transduced with an AAV vector encoding the eZFP-tripartite effector fusion proteins and assessed for FXN expression.

The tripartite effectors of the eZFP fusion proteins comprised, from N-terminus to C-terminus: FOXO3, FOXO3, and NCOA3 (i.e. FOXO3-FOXO3-NCOA3; SEQ ID NO:299); NCOA3, FOXO3, and FOXO3 (i.e. NCOA3-FOXO3-FOXO3; SEQ ID NO:377); or NCOA3, FOXO3, and NCOA3 (i.e. NCOA3-FOXO3-NCOA3; SEQ ID NO:156).

FA-derived cardiomyocytes and neurons were transduced with AAVDJ vectors encoding a FXN-targeting eZFP fused to VP64 or the tripartite effectors, or dSaCas9 fused to VP64 (dSaCas9-2×VP64) or the tripartite effectors and gRNA G. Non-targeting (NT) gRNA and a GFP construct were included as negative controls for FA-derived cells. Cells were harvested on day 7 post-transduction to assess FXN mRNA expression levels by RT-qPCR (normalized to GAPDH) compared to FXN mRNA levels in WT cells.

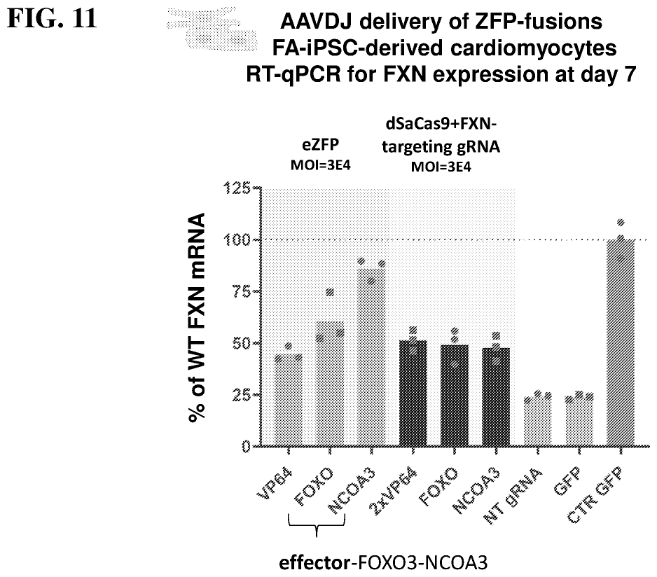

As shown in FIG. 11, all tested fusion proteins with the FXN-targeting eZFP increased FXN mRNA levels in FA-iPSC-derived cardiomyocytes. The eZFP fused with NCOA3-FOXO3-NCOA3 increased FXN mRNA to approximately 80% of WT levels, eZFP fused with FOXO3-FOXO3-NCOA3 increased FXN mRNA to approximately 60% of WT, and eZFP fused with 2×VP64 increased FXN mRNA to approximately 50% of WT. In contrast, the three dSaCas9-effector fusions paired with gRNA G each increased FXN mRNA to approximately 50% of WT in FA-iPSC-derived cardiomyocytes.

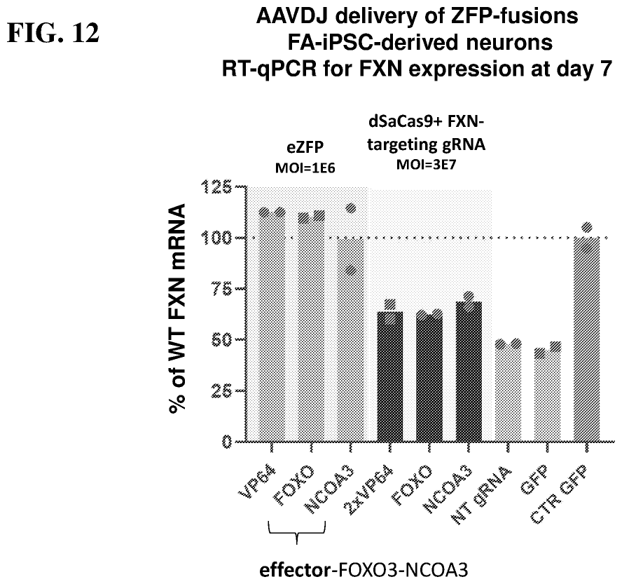

As shown in FIG. 12, all three eZFP effector fusions also increased FXN mRNA levels in FA-iPSC-derived neurons. Notably, FA-neurons expressing the eZFP effector fusion proteins exhibited 100% or more of WT FXN mRNA levels. The dSaCas9-effector fusions paired with gRNA G all increased FXN mRNA levels to approximately 60% of WT in FA-iPSC-derived neurons.

Various formats of a FXN-targeting eZFPtripartite effector fusion were generated with different tripartite effectors fused at the C-terminus or the N-terminus of an exemplary FXN-targeting eZFP, including the following effectors: NCOA3-FOXO3-FOXO3 (e.g. as set forth in SEQ ID NO:377), FOXO3-FOXO3-NCOA3 (e.g. as set forth in SEQ ID NO:299), and NCOA3-FOXO3-NCOA3 (e.g. as set forth in SEQ ID NO:156).

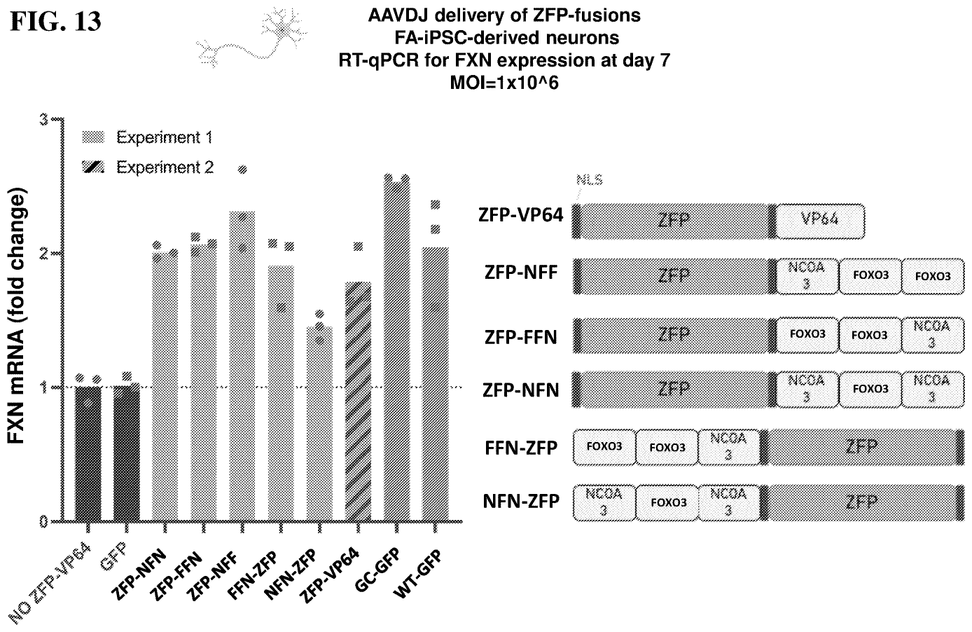

As shown in FIG. 13, all eZFP−tripartite effector fusion protein formats resulted in increased FXN mRNA in FA-neurons. C-terminal fusions of the tripartite effectors led to a stronger increase of FXN mRNA than N-terminal fusions. Further, the C-terminal tripartite fusions increased FXN expression more than FXN-targeting eZFP−VP64 and fully restored FXN mRNA to WT levels in FA-iPSC-derived neurons.

In summary, these results demonstrated that eZFP-tripartite effector fusions targeted an exemplary target gene, e.g., FXN, are potent transcriptional activators and can increase target gene expression levels to a similar or greater extent than dCas9-based transcriptional activation in source cells, such as FA-iPSC-derived cells. The eZFP tripartite effector fusion proteins also increased FXN mRNA levels to greater or similar levels as eZFP−VP64. Notably, all eZFP−effector fusion proteins restored FXN mRNA to 100% or more of WT levels in FA-iPSC-derived neurons. The fusion proteins containing the eZFP and tripartite effectors form a compact and human-derived fusion protein well-suited to AAV delivery, for delivery and targeting of genes in which expression is desired to be increased for treatment of a disease or condition, such as Friedreich ataxia (FA). The results generally support the therapeutic utility of the eZFPs and eZFP fusion proteins provided herein for targeted activation of gene expression.

### Example 9: Multipartite Effector Domains for Targeted Gene Activation in T Cells

DNA-targeting systems with multipartite activators were assessed for ability to activate an exemplary target gene (IL-2) in T cells.

### A. Targeted Gene Activation in T Cells Using Multipartite Activators

Human primary T cells from different donors were thawed and activated with activation reagents (e.g. anti-CD3/anti-CD28 activation reagents) on day 0, then transduced with an anti-Her2 chimeric antigen receptor (CAR) on day 1. On day 4, cells were delivered with DNA-targeting systems including guide RNA (gRNA) and effector fusion proteins containing multipartite effectors, as described below. Delivery was performed by electroporating cells with mRNA encoding the fusion protein and pre-transcribed gRNA.

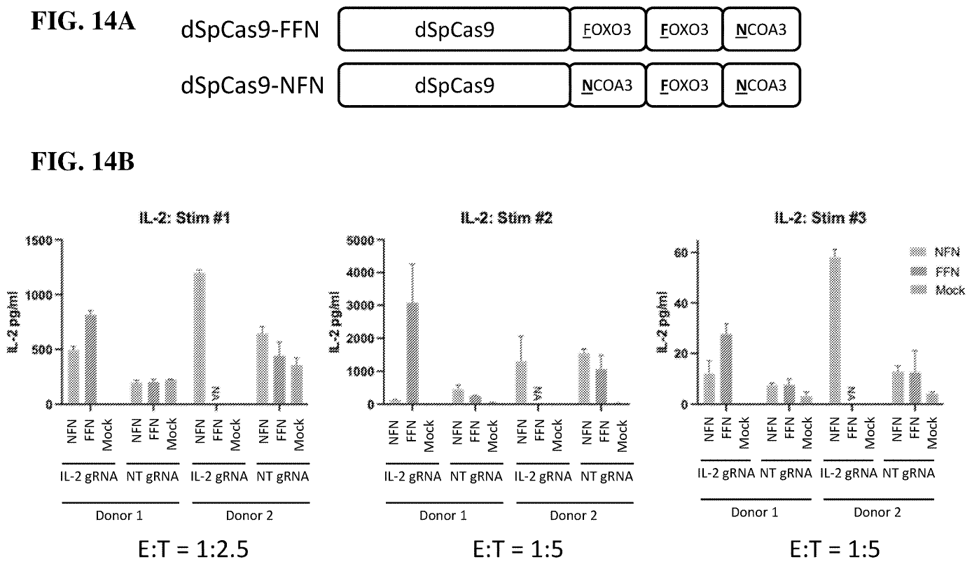

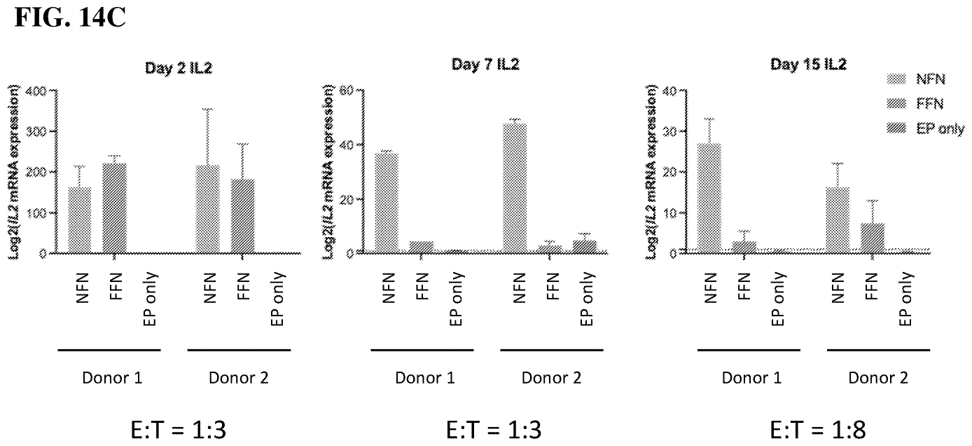

The Her2 CAR T cells were delivered with an IL-2-targeting gRNA and a dSpCas9-fusion protein comprising dSpCas9 (e.g., SEQ ID NO:6) fused at its C-terminus to a FOXO3-FOXO3-NCOA3 tripartite effector (FFN; SEQ ID NO:158), or to a NCOA3-FOXO3-NCOA3 tripartite effector (NFN; SEQ ID NO:156), e.g. as shown in FIG. 14A. Negative control cells received a non-targeting guide RNA (“NT gRNA”), were not transduced with a CAR (“Mock”), or were sham electroporated without the DNA-targeting system (“EP only”).

To assess IL-2 secretion after activation, the Her2 CAR T cells were serially stimulated with a Her2-expressing cell line, for 3 serial stimulations, each approximately 1 week apart. The Her2 CAR T cell:target cell ratio was 1:2.5 for stimulation #1, and 1:5 for simulation #2 and #3. Cells were assessed for IL-2 secretion after each stimulation (e.g., by using Meso Scale Discovery (MSD) immunoassay to measure secreted cytokine in the cell culture medium). Results were obtained using T cells derived from two different donors. As shown in FIG. 14B, stimulated CAR T cells delivered with the DNA-targeting systems with the tripartite effectors targeted to IL-2 exhibited increased secretion of IL-2 in comparison to negative control cells, including after multiple stimulations.

To assess IL-2 mRNA expression after activation, the Her2 CAR T cells were serially stimulated with Her2-expressing cell line, for 3 serial stimulations, each approximately 1 week apart. The Her2 CAR T cell: target cell ratio was 1:3 for stimulation #1 and #2 (assessed at day 2 and day 7 post-EP, respectively), and 1:8 for simulation #3 (assessed at day 15 post-EP). Cells were assessed for IL-2 mRNA expression after each stimulation by RT-qPCR. Results were obtained using T cells derived from two different donors. As shown in FIG. 14C, stimulated CAR T cells delivered with the DNA-targeting systems with tripartite effectors targeted to IL-2 exhibited increased expression of IL-2 mRNA in comparison to negative control cells, including after multiple stimulations.

### B. Targeted Gene Activation in T Cells Using an NCOA3-FOXO3-NCOA3 Multipartite Activator

Human primary T cells from different donors were thawed and activated with activation reagents (e.g. anti-CD3/anti-CD28 activation reagents) on day 0, then transduced with an anti-Her2 chimeric antigen receptor (CAR) on day 1. On day 4, cells were delivered with DNA-targeting systems including an IL-2 gRNA (either one or both of a first and second IL-2 targeting gRNA; IL-2 gRNA 1 or IL-2 gRNA 2, respectively) and a dCas9-fusion protein fused to a NCOA3-FOXO3-NCOA3 tripartite effector (NFN; SEQ ID NO:156). To deliver the DNA-targeting systems, cells were electroporated with mRNA encoding the fusion protein and pre-transcribed gRNA. Negative control cells received a non-targeting gRNA (“NT gRNA”), were not electroporated with a DNA-targeting system (“CAR only”). Cells were allowed to proliferate and were then stimulated with Her2-expressing target cells on day 7 post-EP and day 14 post-EP at a 1:1 ratio of effector CAR T cells: target cells.

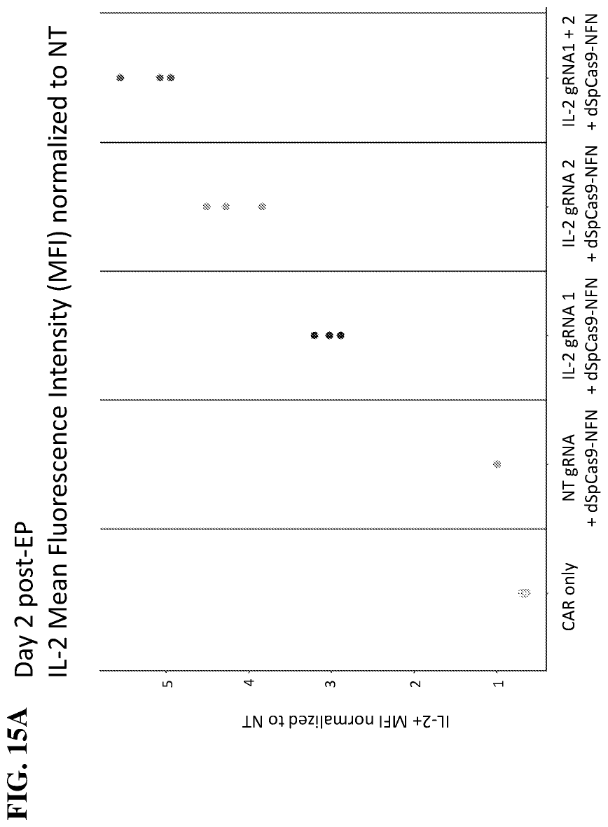

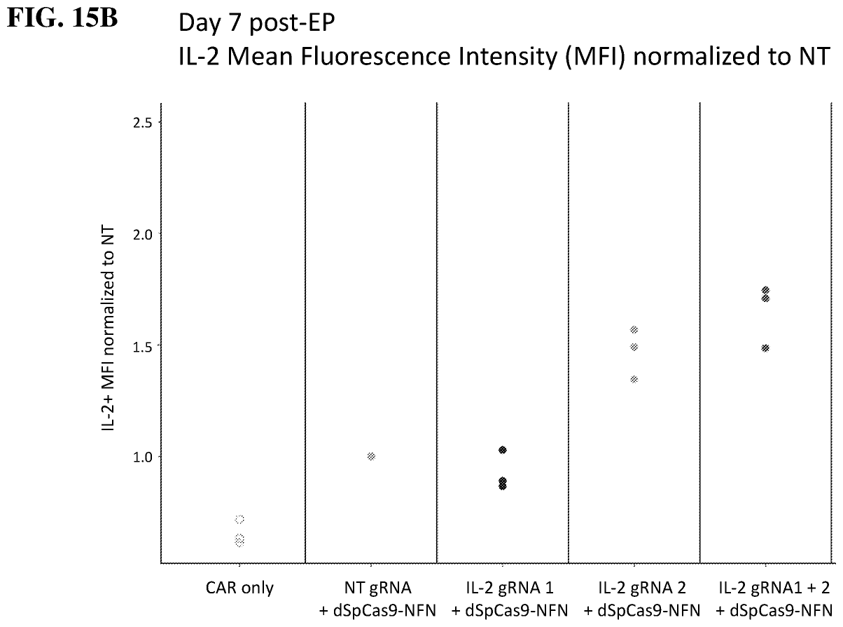

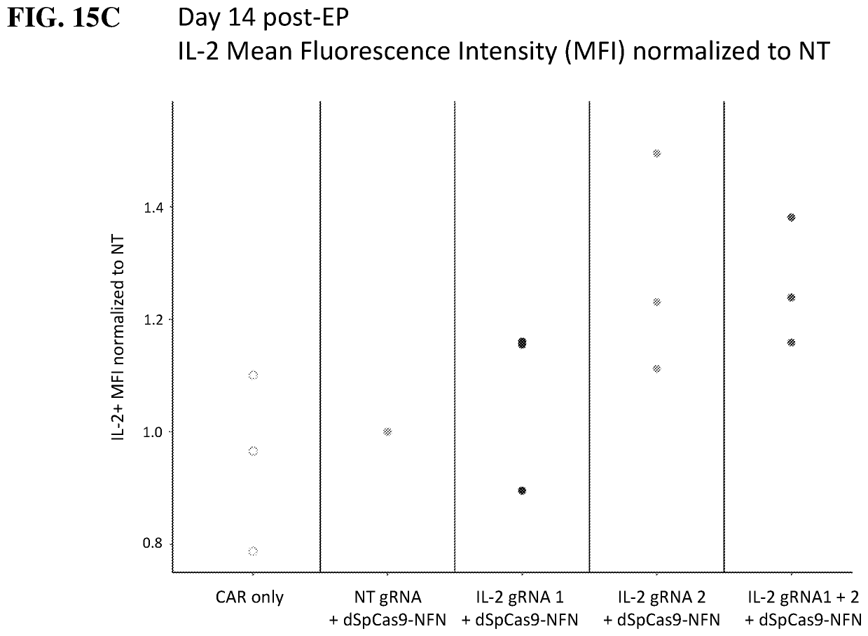

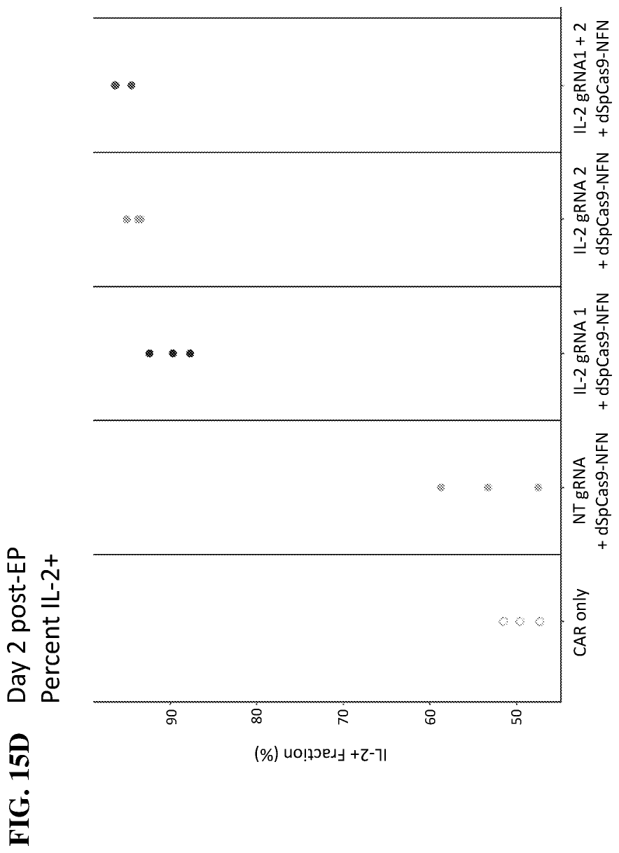

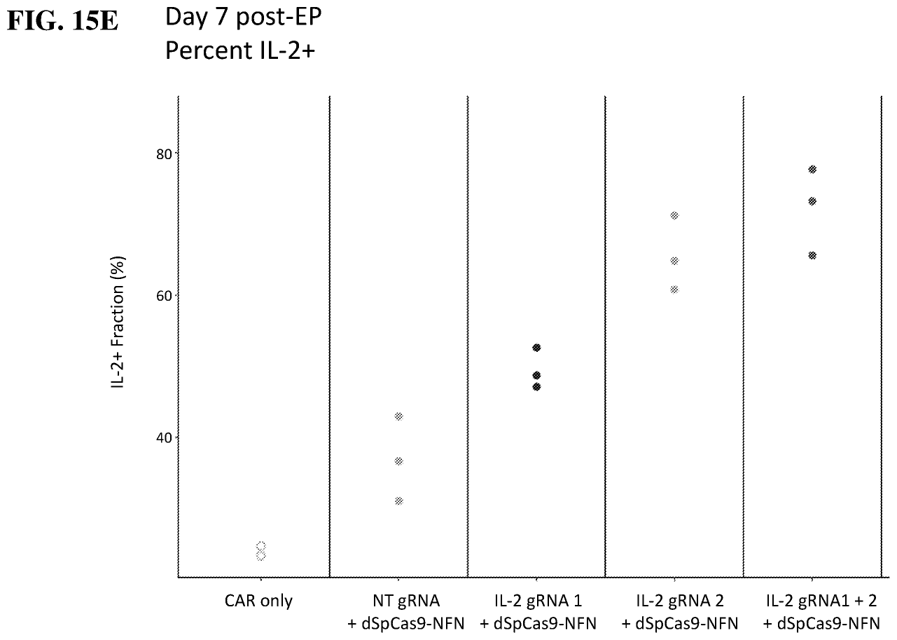

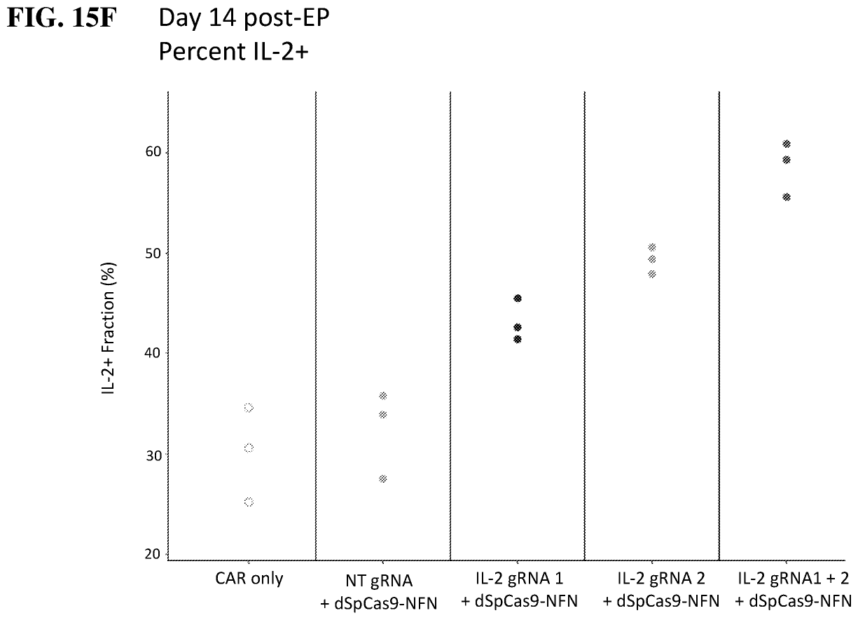

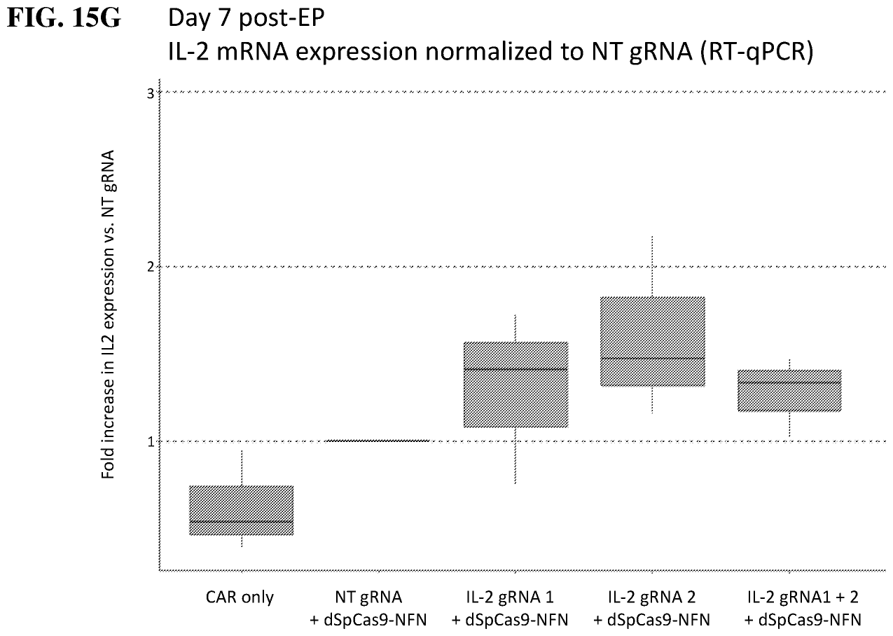

To assess surface IL-2 expression, cells were stained by intracellular cytokine staining (ICS) and assessed by flow cytometry at day 2 post-EP, and 24 hours after the stimulations on day 7 post-EP and day 14 post-EP. IL-2 expression was quantified as mean fluorescence intensity (MFI) normalized to control cells delivered with a non-targeting gRNA (“NT”) (as shown in FIGS. 15A-C), or as the percentage of cells that were positive for IL-2 (as shown in FIGS. 15D-F). Results were obtained for each condition using T cells derived from three different donors. As shown in FIGS. 15A-F, stimulated CAR T cells delivered with the DNA-targeting systems with tripartite effectors targeted to IL-2 exhibited increased surface expression of IL-2 in comparison to control cells for up to 2 weeks post-EP.

The cells were also assessed for IL-2 mRNA expression by RT-qPCR at 7 days post-EP, normalized to negative control cells delivered with a non-targeting gRNA (“NT”). Results were obtained for each condition using T cells derived from three different donors. As shown in FIG. 15G, stimulated CAR T cells delivered with the DNA-targeting systems with tripartite effectors targeted to IL-2 exhibited increased IL-2 mRNA expression in comparison to control cells.

The results demonstrate the utility of the multipartite effectors for targeted transcriptional activation in a variety of cell types, including T cells.

### Example 10: Multipartite Effector Domains for Targeted Activation of Genes in Jurkat Cells

Lymphoid cells (Jurkat cells; an immortalized human lymphoid cell line) were delivered with DNA-targeting systems for activation of an exemplary gene (CCR7) using multipartite activators or combinations of effector domains for targeted transcriptional activation, and assessed for CCR7 expression.

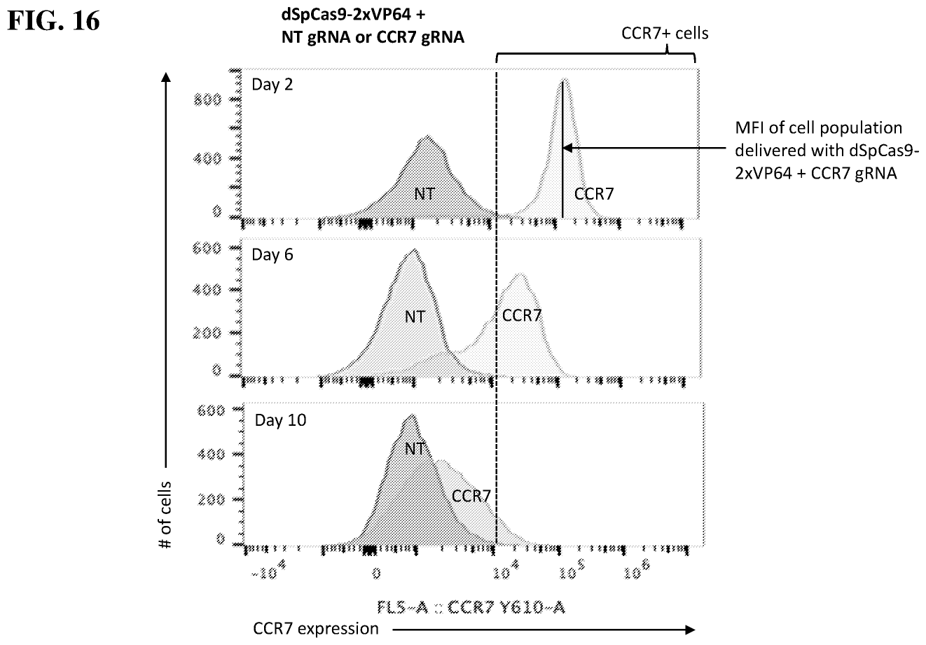

Jurkat cells were delivered with dSpCas9-2×VP64 and a CCR7-targeting gRNA. Negative control cells received a non-targeting gRNA (“NT”). Cells were assessed for CCR7 expression at day 2, day 6, and day 10 post-delivery by flow cytometry. As shown in FIG. 16, Jurkat cells delivered with the CCR7-targeting DNA-targeting system exhibited increased cell surface expression of CCR7 for up to 10 days post-delivery. The histogram curves demonstrate that CCR7 expression can be measured as mean fluorescence intensity (MFI) of a population, or as a percentage of cells that are CCR7 positive.

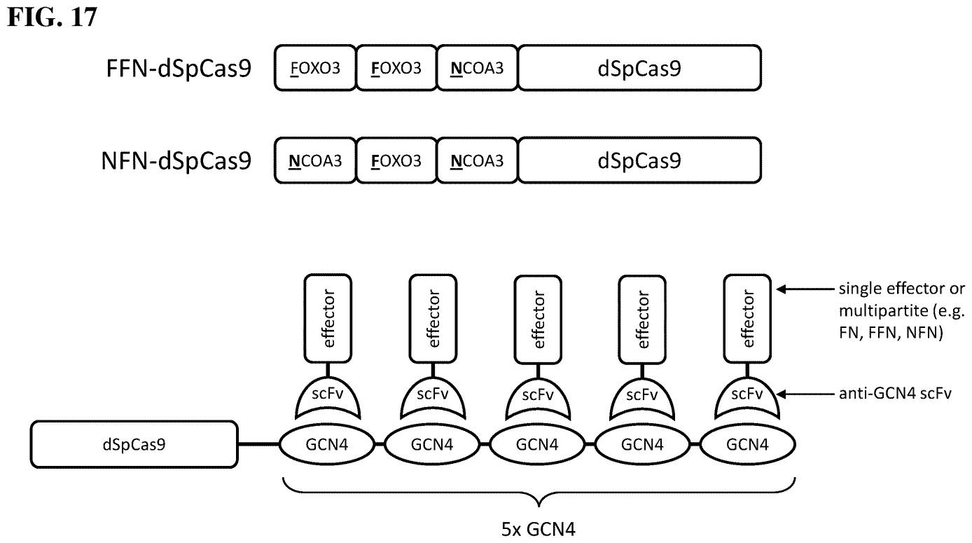

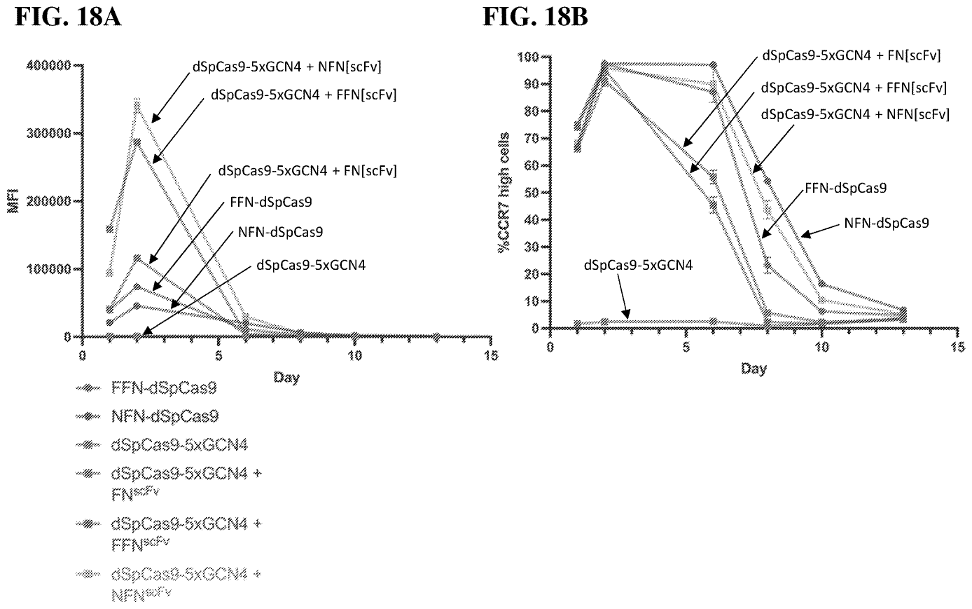

Next, Jurkat cells were delivered with DNA-targeting systems containing a CCR7-targeting gRNA and multipartite effectors for targeted activation, including a FOXO3-FOXO3-NCOA3 tripartite effector (FFN; SEQ ID NO:158), an NCOA3-FOXO3-NCOA3 tripartite effector (NFN; SEQ ID NO:156), or a FOXO3-NCOA3 bipartite effector (FN; SEQ ID NO:144). The effectors were either delivered in a fusion protein with dSpCas9, or in a fusion protein with a single-chain variable fragment (scFv) targeting a repeating GCN4 epitope present in a co-delivered dSpCas9-5×GCN4 fusion protein, as illustrated in FIG. 17. Negative control cells were delivered with the gRNA and dSpCas9-5×GCN4 fusion protein but not an effector for activation. Jurkat cells were assessed for CCR7 expression by flow cytometry at multiple timepoints after delivery. As shown in FIGS. 18A-B, each of the multipartite effectors induced increased expression of CCR7, as measured by CCR7 MFI (FIG. 18A) and % CCR7-high cells (FIG. 18B).

The results demonstrate the utility of the multipartite effectors for targeted transcriptional activation in a variety of cell types, including lymphoid cells.

### Example 11: Targeted Transcriptional Activation with Individual Effector Domains and Combinations Thereof

Jurkat cells (an immortalized human lymphoid cell line) were delivered with DNA-targeting systems for activation of an exemplary gene (CCR7) using individual effector domains or combinations thereof, and assessed for CCR7 expression.

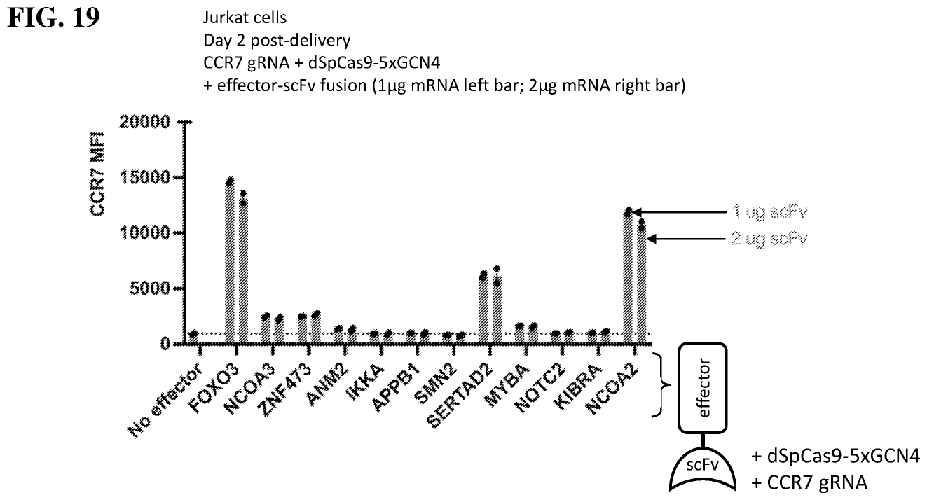

First, individual effector domains were tested for ability to induce targeted activation of the exemplary gene CCR7. Jurkat cells were delivered with DNA-targeting systems containing: a CCR7-targeting gRNA; a fusion protein containing dSpCas9 and a repeating GCN4 epitope array (dSpCas9-5×GCN4); and an effector-scFv fusion protein containing an effector domain set forth in Table E6 and an scFv domain targeting GCN4 (e.g. as shown in FIG. 17). The effector-scFv fusion protein was omitted in negative control cells. For each condition, cells were delivered with either 1 μg or 2 μg of mRNA encoding the effector-scFv fusion protein (shown in FIG. 19 as left and right bars for each condition, respectively).

TABLE E6
Individual effector domains from effector-scFv fusion proteins

| Effector | SEQ ID NO: |
|---|---|
| FOXO3 | 24 |
| NCOA3 | 27 |
| ZNF473 | 378 |
| ANM2 | 379 |
| IKKA | 382 |
| APPB1 | 383 |
| SMN2 | 384 |
| SERTAD2 | 385 |
| MYBA | 386 |
| NOTCH2 | 381 |
| KIBRA | 380 |
| NCOA2 | 26 |

2 days after delivery of the DNA-targeting systems, CCR7 expression was assessed by flow cytometry and determination of mean fluorescence intensity of the signal corresponding to CCR7 (CCR7 MFI). As shown in FIG. 19, several of the effector domains induced activation of CCR7 expression in comparison to control cells.

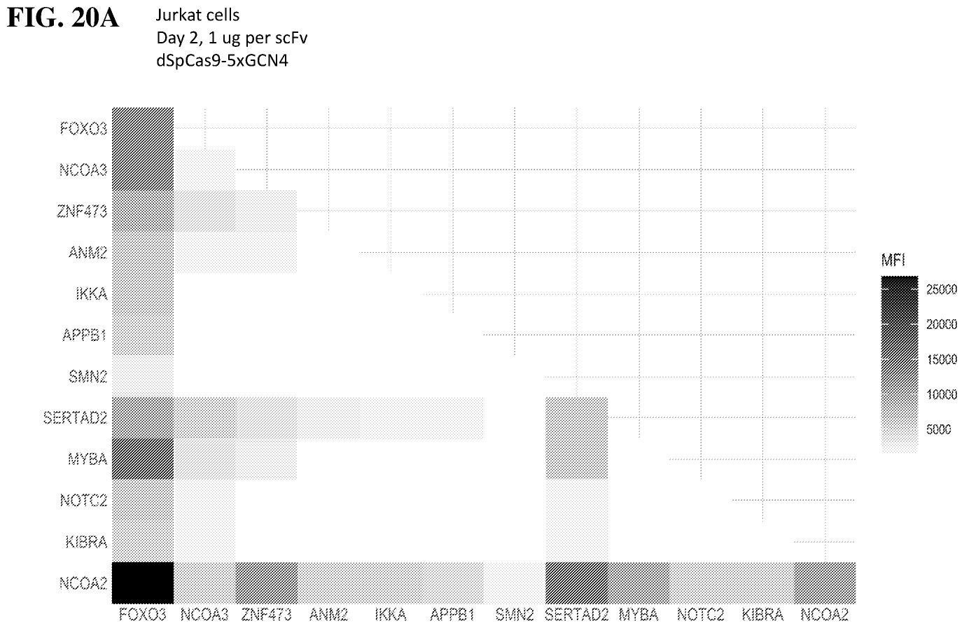

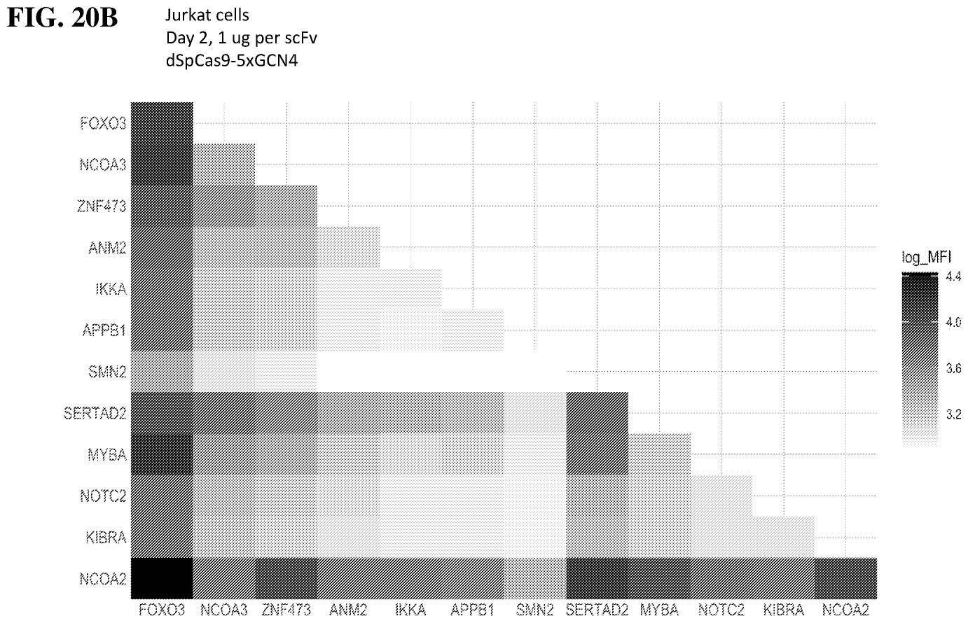

Next, combinations of the effector domains were tested for ability to induce targeted activation of the exemplary gene CCR7. To test combinations of the effector domains, Jurkat cells were delivered with DNA-targeting systems containing: a CCR7-targeting gRNA; a fusion protein containing dSpCas9 and a repeating GCN4 epitope array (dSpCas9-5×GCN4); and a first and second effector-scFv fusion protein each containing an effector domain and an scFv domain targeting GCN4. The effector domains of the first and second effector-scFv fusion proteins were each independently selected from one of the 12 effector domains shown in Table E6, with 144 different combinations of effector domains being tested. 2 days after delivery of the DNA-targeting systems, CCR7 expression was assessed by flow cytometry and determination of mean fluorescence intensity of the signal corresponding to CCR7 (CCR7 MFI). FIGS. 20A-B show heatmaps indicating CCR7 MFI for each combination of effector domains tested, with MFI shown on a linear scale (FIG. 20A) or log scale (FIG. 20B). The results show that several combinations of effector domains lead to increased CCR7 expression, including FOXO3+NCOA2, FOXO3+MYBA, SERTAD2+NCOA2, and FOXO3+NCOA3.

Together, the data support the utility of the tested effector domains, alone or in combinations (e.g. in multipartite effector fusion proteins), for targeted transcriptional activation in a variety of cell types, including lymphoid cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | AAGCGGAACTACATCCTGGGCCTGGCCATCGGCATCACCAGCGTGGGCTACGGCATCA | dSaCas9 (nt) |
| | TCGACTACGAGACACGGGACGTGATCGATGCCGGCGTGCGGCTGTTCAAAGAGGCCAA | |
| | CGTGGAAAACAACGAGGGCAGGCGGAGCAAGAGAGGCGCCAGAAGGCTGAAGCGGCGG | |
| | AGGCGGCATAGAATCCAGAGAGTGAAGAAGCTGCTGTTCGACTACAACCTGCTGACCG | |
| | ACCACAGCGAGCTGAGCGGCATCAACCCCTACGAGGCCAGAGTGAAGGGCCTGAGCCA | |
| | GAAGCTGAGCGAGGAAGAGTTCTCTGCCGCCCTGCTGCACCTGGCCAAGAGAAGAGGC | |
| | GTGCACAACGTGAACGAGGTGGAAGAGGACACCGGCAACGAGCTGTCCACCAAAGAGC | |
| | AGATCAGCCGGAACAGCAAGGCCCTGGAAGAGAAATACGTGGCCGAACTGCAGCTGGA | |
| | ACGGCTGAAGAAAGACGGCGAAGTGCGGGGCAGCATCAACAGATTCAAGACCAGCGAC | |
| | TACGTGAAAGAAGCCAAACAGCTGCTGAAGGTGCAGAAGGCCTACCACCAGCTGGACC | |
| | AGAGCTTCATCGACACCTACATCGACCTGCTGGAAACCCGGCGGACCTACTATGAGGG | |
| | ACCTGGCGAGGGCAGCCCCTTCGGCTGGAAGGACATCAAAGAATGGTACGAGATGCTG | |
| | ATGGGCCACTGCACCTACTTCCCCGAGGAACTGCGGAGCGTGAAGTACGCCTACAACG | |
| | CCGACCTGTACAACGCCCTGAACGACCTGAACAATCTCGTGATCACCAGGGACGAGAA | |
| | CGAGAAGCTGGAATATTACGAGAAGTTCCAGATCATCGAGAACGTGTTCAAGCAGAAG | |
| | AAGAAGCCCACCCTGAAGCAGATCGCCAAAGAAATCCTCGTGAACGAAGAGGATATTA | |
| | AGGGCTACAGAGTGACCAGCACCGGCAAGCCCGAGTTCACCAACCTGAAGGTGTACCA | |
| | CGACATCAAGGACATTACCGCCCGGAAAGAGATTATTGAGAACGCCGAGCTGCTGGAT | |
| | CAGATTGCCAAGATCCTGACCATCTACCAGAGCAGCGAGGACATCCAGGAAGAACTGA | |
| | CCAATCTGAACTCCGAGCTGACCCAGGAAGAGATCGAGCAGATCTCTAATCTGAAGGG | |
| | CTATACCGGCACCCACAACCTGAGCCTGAAGGCCATCAACCTGATCCTGGACGAGCTG | |
| | TGGCACACCAACGACAACCAGATCGCTATCTTCAACCGGCTGAAGCTGGTGCCCAAGA | |
| | AGGTGGACCTGTCCCAGCAGAAAGAGATCCCCACCACCCTGGTGGACGACTTCATCCT | |
| | GAGCCCCGTCGTGAAGAGAAGCTTCATCCAGAGCATCAAAGTGATCAACGCCATCATC | |
| | AAGAAGTACGGCCTGCCCAACGACATCATTATCGAGCTGGCCCGCGAGAAGAACTCCA | |
| | AGGACGCCCAGAAAATGATCAACGAGATGCAGAAGCGGAACCGGCAGACCAACGAGCG | |
| | GATCGAGGAAATCATCCGGACCACCGGCAAAGAGAACGCCAAGTACCTGATCGAGAAG | |
| | ATCAAGCTGCACGACATGCAGGAAGGCAAGTGCCTGTACAGCCTGGAAGCCATCCCTC | |
| | TGGAAGATCTGCTGAACAACCCCTTCAACTATGAGGTGGACCACATCATCCCCAGAAG | |
| | CGTGTCCTTCGACAACAGCTTCAACAACAAGGTGCTCGTGAAGCAGGAAGAAgcCAGC | |
| | AAGAAGGGCAACCGGACCCCATTCCAGTACCTGAGCAGCAGCGACAGCAAGATCAGCT | |
| | ACGAAACCTTCAAGAAGCACATCCTGAATCTGGCCAAGGGCAAGGGCAGAATCAGCAA | |
| | GACCAAGAAAGAGTATCTGCTGGAAGAACGGGACATCAACAGGTTCTCCGTGCAGAAA | |
| | GACTTCATCAACCGGAACCTGGTGGATACCAGATACGCCACCAGAGGCCTGATGAACC | |
| | TGCTGCGGAGCTACTTCAGAGTGAACAACCTGGACGTGAAAGTGAAGTCCATCAATGG | |
| | CGGCTTCACCAGCTTTCTGCGGCGGAAGTGGAAGTTTAAGAAAGAGCGGAACAAGGGG | |
| | TACAAGCACCACGCCGAGGACGCCCTGATCATTGCCAACGCCGATTTCATCTTCAAAG | |
| | AGTGGAAGAAACTGGACAAGGCCAAAAAAGTGATGGAAAACCAGATGTTCGAGGAAAA | |
| | GCAGGCCGAGAGCATGCCCGAGATCGAAACCGAGCAGGAGTACAAAGAGATCTTCATC | |
| | ACCCCCCACCAGATCAAGCACATTAAGGACTTCAAGGACTACAAGTACAGCCACCGGG | |
| | TGGACAAGAAGCCTAATAGAGAGCTGATTAACGACACCCTGTACTCCACCCGGAAGGA | |
| | CGACAAGGGCAACACCCTGATCGTGAACAATCTGAACGGCCTGTACGACAAGGACAAT | |
| | GACAAGCTGAAAAAGCTGATCAACAAGAGCCCCGAAAAGCTGCTGATGTACCACCACG | |
| | ACCCCCAGACCTACCAGAAACTGAAGCTGATTATGGAACAGTACGGCGACGAGAAGAA | |
| | TCCCCTGTACAAGTACTACGAGGAAACCGGGAACTACCTGACCAAGTACTCCAAAAAG | |
| | GACAACGGCCCCGTGATCAAGAAGATTAAGTATTACGGCAACAAACTGAACGCCCATC | |
| | TGGACATCACCGACGACTACCCCAACAGCAGAAACAAGGTCGTGAAGCTGTCCCTGAA | |
| | GCCCTACAGATTCGACGTGTACCTGGACAATGGCGTGTACAAGTTCGTGACCGTGAAG | |
| | AATCTGGATGTGATCAAAAAAGAAAACTACTACGAAGTGAATAGCAAGTGCTATGAGG | |
| | AAGCTAAGAAGCTGAAGAAGATCAGCAACCAGGCCGAGTTTATCGCCTCCTTCTACAA | |
| | CAACGATCTGATCAAGATCAACGGCGAGCTGTATAGAGTGATCGGCGTGAACAACGAC | |
| | CTGCTGAACCGGATCGAAGTGAACATGATCGACATCACCTACCGCGAGTACCTGGAAA | |
| | ACATGAACGACAAGAGGCCCCCCAGGATCATTAAGACAATCGCCTCCAAGACCCAGAG | |
| | CATTAAGAAGTACAGCACAGACATTCTGGGCAACCTGTATGAAGTGAAATCTAAGAAG | |
| | CACCCTCAGATCATCAAAAAGGGC | |
| 2 | KRNYILGLAIGITSVGYGIIDYETRDVIDAGVRLFKEANVENNEGRRSKRGARRLKRR | dSaCas9 (aa) |
| | RRHRIQRVKKLLFDYNLLTDHSELSGINPYEARVKGLSQKLSEEEFSAALLHLAKRRG | |
| | VHNVNEVEEDTGNELSTKEQISRNSKALEEKYVAELQLERLKKDGEVRGSINRFKTSD | |
| | YVKEAKQLLKVQKAYHQLDQSFIDTYIDLLETRRTYYEGPGEGSPFGWKDIKEWYEML | |
| | MGHCTYFPEELRSVKYAYNADLYNALNDLNNLVITRDENEKLEYYEKFQIIENVFKQK | |
| | KKPTLKQIAKEILVNEEDIKGYRVTSTGKPEFTNLKVYHDIKDITARKEIIENAELLD | |
| | QIAKILTIYQSSEDIQEELTNLNSELTQEEIEQISNLKGYTGTHNLSLKAINLILDEL | |
| | WHTNDNQIAIFNRLKLVPKKVDLSQQKEIPTTLVDDFILSPVVKRSFIQSIKVINAII | |
| | KKYGLPNDIIIELAREKNSKDAQKMINEMQKRNRQTNERIEEIIRTTGKENAKYLIEK | |
| | IKLHDMQEGKCLYSLEAIPLEDLLNNPFNYEVDHIIPRSVSFDNSENNKVLVKQEEAS | |
| | KKGNRTPFQYLSSSDSKISYETFKKHILNLAKGKGRISKTKKEYLLEERDINRFSVQK | |
| | DFINRNLVDTRYATRGLMNLLRSYFRVNNLDVKVKSINGGFTSFLRRKWKFKKERNKG | |
| | YKHHAEDALIIANADFIFKEWKKLDKAKKVMENQMFEEKQAESMPEIETEQEYKEIFI | |
| | TPHQIKHIKDFKDYKYSHRVDKKPNRELINDTLYSTRKDDKGNTLIVNNLNGLYDKDN | |
| | DKLKKLINKSPEKLLMYHHDPQTYQKLKLIMEQYGDEKNPLYKYYEETGNYLTKYSKK | |
| | DNGPVIKKIKYYGNKLNAHLDITDDYPNSRNKVVKLSLKPYRFDVYLDNGVYKFVTVK | |
| | NLDVIKKENYYEVNSKCYEEAKKLKKISNQAEFIASFYNNDLIKINGELYRVIGVNND | |
| | LLNRIEVNMIDITYREYLENMNDKRPPRIIKTIASKTQSIKKYSTDILGNLYEVKSKK | |
| | HPQIIKKG | |
| 3 | MKRNYILGLDIGITSVGYGIIDYETRDVIDAGVRLFKEANVENNEGRRSKRGARRLKR | WT Sa Cas9 (aa) |
| | RRRHRIQRVKKLLFDYNLLTDHSELSGINPYEARVKGLSQKLSEEEFSAALLHLAKRR | |
| | GVHNVNEVEEDTGNELSTKEQISRNSKALEEKYVAELQLERLKKDGEVRGSINRFKTS | |
| | DYVKEAKQLLKVQKAYHQLDQSFIDTYIDLLETRRTYYEGPGEGSPFGWKDIKEWYEM | |
| | LMGHCTYFPEELRSVKYAYNADLYNALNDLNNLVITRDENEKLEYYEKFQIIENVFKQ | |
| | KKKPTLKQIAKEILVNEEDIKGYRVTSTGKPEFTNLKVYHDIKDITARKEIIENAELL | |
| | DQIAKILTIYQSSEDIQEELTNLNSELTQEEIEQISNLKGYTGTHNLSLKAINLILDE | |
| | LWHTNDNQIAIFNRLKLVPKKVDLSQQKEIPTTLVDDFILSPVVKRSFIQSIKVINAI | |
| | IKKYGLPNDIIIELAREKNSKDAQKMINEMQKRNRQTNERIEEIIRTTGKENAKYLIE | |
| | KIKLHDMQEGKCLYSLEAIPLEDLLNNPFNYEVDHIIPRSVSFDNSFNNKVLVKQEEN | |
| | SKKGNRTPFQYLSSSDSKISYETFKKHILNLAKGKGRISKTKKEYLLEERDINRFSVQ | |
| | KDFINRNLVDTRYATRGLMNLLRSYFRVNNLDVKVKSINGGFTSFLRRKWKFKKERNK | |
| | GYKHHAEDALIIANADFIFKEWKKLDKAKKVMENQMFEEKQAESMPEIETEQEYKEIF | |
| | ITPHQIKHIKDFKDYKYSHRVDKKPNRELINDTLYSTRKDDKGNTLIVNNLNGLYDKD | |
| | NDKLKKLINKSPEKLLMYHHDPQTYQKLKLIMEQYGDEKNPLYKYYEETGNYLTKYSK | |
| | KDNGPVIKKIKYYGNKLNAHLDITDDYPNSRNKVVKLSLKPYRFDVYLDNGVYKFVTV | |
| | KNLDVIKKENYYEVNSKCYEEAKKLKKISNQAEFIASFYNNDLIKINGELYRVIGVNN | |
| | DLLNRIEVNMIDITYREYLENMNDKRPPRIIKTIASKTQSIKKYSTDILGNLYEVKSK | |
| | KHPQIIKKG | |
| 4 | KRNYILGLDIGITSVGYGIIDYETRDVIDAGVRLFKEANVENNEGRRSKRGARRLKRR | WT Sa Cas9 (aa) |
| | RRHRIQRVKKLLFDYNLLTDHSELSGINPYEARVKGLSQKLSEEEFSAALLHLAKRRG | |
| | VHNVNEVEEDTGNELSTKEQISRNSKALEEKYVAELQLERLKKDGEVRGSINRFKTSD | |
| | YVKEAKQLLKVQKAYHQLDQSFIDTYIDLLETRRTYYEGPGEGSPFGWKDIKEWYEML | |
| | MGHCTYFPEELRSVKYAYNADLYNALNDLNNLVITRDENEKLEYYEKFQIIENVFKQK | |
| | KKPTLKQIAKEILVNEEDIKGYRVTSTGKPEFTNLKVYHDIKDITARKEIIENAELLD | |
| | QIAKILTIYQSSEDIQEELTNLNSELTQEEIEQISNLKGYTGTHNLSLKAINLILDEL | |
| | WHTNDNQIAIFNRLKLVPKKVDLSQQKEIPTTLVDDFILSPVVKRSFIQSIKVINAII | |
| | KKYGLPNDIIIELAREKNSKDAQKMINEMQKRNRQTNERIEEIIRTTGKENAKYLIEK | |
| | IKLHDMQEGKCLYSLEAIPLEDLLNNPFNYEVDHIIPRSVSFDNSENNKVLVKQEENS | |
| | KKGNRTPFQYLSSSDSKISYETFKKHILNLAKGKGRISKTKKEYLLEERDINRFSVQK | |
| | DFINRNLVDTRYATRGLMNLLRSYFRVNNLDVKVKSINGGFTSFLRRKWKFKKERNKG | |
| | YKHHAEDALIIANADFIFKEWKKLDKAKKVMENQMFEEKQAESMPEIETEQEYKEIFI | |
| | TPHQIKHIKDFKDYKYSHRVDKKPNRELINDTLYSTRKDDKGNTLIVNNLNGLYDKDN | |
| | DKLKKLINKSPEKLLMYHHDPQTYQKLKLIMEQYGDEKNPLYKYYEETGNYLTKYSKK | |
| | DNGPVIKKIKYYGNKLNAHLDITDDYPNSRNKVVKLSLKPYRFDVYLDNGVYKFVTVK | |
| | NLDVIKKENYYEVNSKCYEEAKKLKKISNQAEFIASFYNNDLIKINGELYRVIGVNND | |
| | LLNRIEVNMIDITYREYLENMNDKRPPRIIKTIASKTQSIKKYSTDILGNLYEVKSKK | |
| | HPQIIKKG | |
| 5 | GACAAGAAGTACTCCATTGGGCTCGCCATCGGCACAAACAGCGTCGGCTGGGCCGTCA | dSpCas9 (nt) |
| | TTACGGACGAGTACAAGGTGCCGAGCAAAAAATTCAAAGTTCTGGGCAATACCGATCG | |
| | CCACAGCATAAAGAAGAACCTCATTGGCGCCCTCCTGTTCGACTCCGGGGAAACCGCC | |
| | GAAGCCACGCGGCTCAAAAGAACAGCACGGCGCAGATATACCCGCAGAAAGAATCGGA | |
| | TCTGCTACCtgcaGGAGATCTTTAGTAATGAGATGGCTAAGGTGGATGACTCTTTCTT | |
| | CCATAGGCTGGAGGAGTCCTTTTTGGTGGAGGAGGATAAAAAGCACGAGCGCCACCCA | |
| | ATCTTTGGCAATATCGTGGACGAGGTGGCGTACCATGAAAAGTACCCAACCATATATC | |
| | ATCTGAGGAAGAAGCTTGTAGACAGTACTGATAAGGCTGACTTGCGGTTGATCTATCT | |
| | CGCGCTGGCGCATATGATCAAATTTCGGGGACACTTCCTCATCGAGGGGGACCTGAAC | |
| | CCAGACAACAGCGATGTCGACAAACTCTTTATCCAACTGGTTCAGACTTACAATCAGC | |
| | TTTTCGAAGAGAACCCGATCAACGCATCCGGAGTTGACGCCAAAGCAATCCTGAGCGC | |
| | TAGGCTGTCCAAATCCCGGCGGCTCGAAAACCTCATCGCACAGCTCCCTGGGGAGAAG | |
| | AAGAACGGCCTGTTTGGTAATCTTATCGCCCTGTCACTCGGGCTGACCCCCAACTTTA | |
| | AATCTAACTTCGACCTGGCCGAAGATGCCAAGCTTCAACTGAGCAAAGACACCTACGA | |
| | TGATGATCTCGACAATCTGCTGGCCCAGATCGGCGACCAGTACGCAGACCTTTTTTTG | |
| | GCGGCAAAGAACCTGTCAGACGCCATTCTGCTGAGTGATATTCTGCGAGTGAACACGG | |
| | AGATCACCAAAGCTCCGCTGAGCGCTAGTATGATCAAGCGCTATGATGAGCACCACCA | |
| | AGACTTGACTTTGCTGAAGGCCCTTGTCAGACAGCAACTGCCTGAGAAGTACAAGGAA | |
| | ATTTTCTTCGATCAGTCTAAAAATGGCTACGCCGGATACATTGACGGCGGAGCAAGCC | |
| | AGGAGGAATTTTACAAATTTATTAAGCCCATCTTGGAAAAAATGGACGGCACCGAGGA | |
| | GCTGCTGGTAAAGCTTAACAGAGAAGATCTGTTGCGCAAACAGCGCACTTTCGACAAT | |
| | GGAAGCATCCCCCACCAGATTCACCTGGGCGAACTGCACGCTATCCTCAGGCGGCAAG | |
| | AGGATTTCTACCCCTTTTTGAAAGATAACAGGGAAAAGATTGAGAAAATCCTCACATT | |
| | TCGGATACCCTACTATGTAGGCCCCCTCGCCCGGGGAAATTCCAGATTCGCGTGGATG | |
| | ACTCGCAAATCAGAAGAGACCATCACTCCCTGGAACTTCGAGGAAGTCGTGGATAAGG | |
| | GGGCCTCTGCCCAGTCCTTCATCGAAAGGATGACTAACTTTGATAAAAATCTGCCTAA | |
| | CGAAAAGGTGCTTCCTAAACACTCTCTGCTGTACGAGTACTTCACAGTTTATAACGAG | |
| | CTCACCAAGGTCAAATACGTCACAGAAGGGATGAGAAAGCCAGCATTCCTGTCTGGAG | |
| | AGCAGAAGAAAGCTATCGTGGACCTCCTCTTCAAGACGAACCGGAAAGTTACCGTGAA | |
| | ACAGCTCAAAGAAGACTATTTCAAAAAGATTGAATGTTTCGACTCTGTTGAAATCAGC | |
| | GGAGTGGAGGATCGCTTCAACGCATCCCTGGGAACGTATCACGATCTCCTGAAAATCA | |
| | TTAAAGACAAGGACTTCCTGGACAATGAGGAGAACGAGGACATTCTTGAGGACATTGT | |
| | CCTCACCCTTACGTTGTTTGAAGATAGGGAGATGATTGAAGAACGCTTGAAAACTTAC | |
| | GCTCATCTCTTCGACGACAAAGTCATGAAACAGCTCAAGAGGCGCCGATATACAGGAT | |
| | GGGGGCGGCTGTCAAGAAAACTGATCAATGGgatcCGAGACAAGCAGAGTGGAAAGAC | |
| | AATCCTGGATTTTCTTAAGTCCGATGGATTTGCCAACCGGAACTTCATGCAGTTGATC | |
| | CATGATGACTCTCTCACCTTTAAGGAGGACATCCAGAAAGCACAAGTTTCTGGCCAGG | |
| | GGGACAGTCTTCACGAGCACATCGCTAATCTTGCAGGTAGCCCAGCTATCAAAAAGGG | |
| | AATACTGCAGACCGTTAAGGTCGTGGATGAACTCGTCAAAGTAATGGGAAGGCATAAG | |
| | CCCGAGAATATCGTTATCGAGATGGCCCGAGAGAACCAAACTACCCAGAAGGGACAGA | |
| | AGAACAGTAGGGAAAGGATGAAGAGGATTGAAGAGGGTATAAAAGAACTGGGGTCCCA | |
| | AATCCTTAAGGAACACCCAGTTGAAAACACCCAGCTTCAGAATGAGAAGCTCTACCTG | |
| | TACTACCTGCAGAACGGCAGGGACATGTACGTGGATCAGGAACTGGACATCAATCGGC | |
| | TCTCCGACTACGACGTGGATGCCATCGTGCCCCAGTCTTTTCTCAAAGATGATTCTAT | |
| | TGATAATAAAGTGTTGACAAGATCCGATAAAAATAGAGGGAAGAGTGATAACGTCCCC | |
| | TCAGAAGAAGTTGTCAAGAAAATGAAAAATTATTGGCGGCAGCTGCTGAACGCCAAAC | |
| | TGATCACACAACGGAAGTTCGATAATCTGACTAAGGCTGAACGAGGTGGCCTGTCTGA | |
| | GTTGGATAAAGCCGGCTTCATCAAAAGGCAGCTTGTTGAGACACGCCAGATCACCAAg | |
| | cacGTGGCCCAAATTCTCGATTCACGCATGAACACCAAGTACGATGAAAATGACAAAC | |
| | TGATTCGAGAGGTGAAAGTTATTACTCTGAAGTCTAAGCTGGTCTCAGATTTCAGAAA | |
| | GGACTTTCAGTTTTATAAGGTGAGAGAGATCAACAATTACCACCATGCGCATGATGCC | |
| | TACCTGAATGCAGTGGTAGGCACTGCACTTATCAAAAAATATCCCAAGCTTGAATCTG | |
| | AATTTGTTTACGGAGACTATAAAGTGTACGATGTTAGGAAAATGATCGCAAAGTCTGA | |
| | GCAGGAAATAGGCAAGGCCACCGCTAAGTACTTCTTTTACAGCAATATTATGAATTTT | |
| | TTCAAGACCGAGATTACACTGGCCAATGGAGAGATTCGGAAGCGACCACTTATCGAAA | |
| | CAAACGGAGAAACAGGAGAAATCGTGTGGGACAAGGGTAGGGATTTCGCGACAGTCCG | |
| | GAAGGTCCTGTCCATGCCGCAGGTGAACATCGTTAAAAAGACCGAAGTACAGACCGGA | |
| | GGCTTCTCCAAGGAAAGTATCCTCCCGAAAAGGAACAGCGACAAGCTGATCGCACGCA | |
| | AAAAAGATTGGGACCCCAAGAAATACGGCGGATTCGATTCTCCTACAGTCGCTTACAG | |
| | TGTACTGGTTGTGGCCAAAGTGGAGAAAGGGAAGTCTAAAAAACTCAAAAGCGTCAAG | |
| | GAACTGCTGGGCATCACAATCATGGAGCGATCAAGCTTCGAAAAAAACCCCATCGACT | |
| | TTCTCGAGGCGAAAGGATATAAAGAGGTCAAAAAAGACCTCATCATTAAGCTTCCCAA | |
| | GTACTCTCTCTTTGAGCTTGAAAACGGCCGGAAACGAATGCTCGCTAGTGCGGGCGAG | |
| | CTGCAGAAAGGTAACGAGCTGGCACTGCCCTCTAAATACGTTAATTTCTTGTATCTGG | |
| | CCAGCCACTATGAAAAGCTCAAAGGGTCTCCCGAAGATAATGAGCAGAAGCAGCTGTT | |
| | CGTGGAACAACACAAACACTACCTTGATGAGATCATCGAGCAAATAAGCGAATTCTCC | |
| | AAAAGAGTGATCCTCGCCGACGCTAACCTCGATAAGGTGCTTTCTGCTTACAATAAGC | |
| | ACAGGGATAAGCCCATCAGGGAGCAGGCAGAAAACATTATCCACTTGTTTACTCTGAC | |
| | CAACTTGGGCGCGCCTGCAGCCTTCAAGTACTTCGACACCACCATAGACAGAAAGCGG | |
| | TACACCTCTACAAAGGAGGTCCTGGACGCCACACTGATTCATCAGTCAATTACGGGGC | |
| | TCTATGAAACAAGAATCGACCTCTCTCAGCTCGGTGGAGAC | |
| 6 | DKKYSIGLAIGTNSVGWAVITDEYKVPSKKFKVLGNTDRHSIKKNLIGALLFDSGETA | dSpCas9 (aa) |
| | EATRLKRTARRRYTRRKNRICYLQEIFSNEMAKVDDSFFHRLEESFLVEEDKKHERHP | |
| | IFGNIVDEVAYHEKYPTIYHLRKKLVDSTDKADLRLIYLALAHMIKFRGHFLIEGDLN | |
| | PDNSDVDKLFIQLVQTYNQLFEENPINASGVDAKAILSARLSKSRRLENLIAQLPGEK | |
| | KNGLFGNLIALSLGLTPNFKSNFDLAEDAKLQLSKDTYDDDLDNLLAQIGDQYADLFL | |
| | AAKNLSDAILLSDILRVNTEITKAPLSASMIKRYDEHHQDLTLLKALVRQQLPEKYKE | |
| | IFFDQSKNGYAGYIDGGASQEEFYKFIKPILEKMDGTEELLVKLNREDLLRKQRTEDN | |
| | GSIPHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTVYNE | |
| | LTKVKYVTEGMRKPAFLSGEQKKAIVDLLFKTNRKVTVKQLKEDYFKKIECFDSVEIS | |
| | GVEDRFNASLGTYHDLLKIIKDKDFLDNEENEDILEDIVLTLTLFEDREMIEERLKTY | |
| | AHLFDDKVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKVMGRHK | |
| | PENIVIEMARENQTTQKGQKNSRERMKRIEEGIKELGSQILKEHPVENTQLQNEKLYL | |
| | YYLQNGRDMYVDQELDINRLSDYDVDAIVPQSFLKDDSIDNKVLTRSDKNRGKSDNVP | |
| | SEEVVKKMKNYWRQLLNAKLITQRKFDNLTKAERGGLSELDKAGFIKRQLVETRQITK | |
| | HVAQILDSRMNTKYDENDKLIREVKVITLKSKLVSDFRKDFQFYKVREINNYHHAHDA | |
| | YLNAVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAGE | |
| | LQKGNELALPSKYVNFLYLASHYEKLKGSPEDNEQKQLFVEQHKHYLDEIIEQISEFS | |
| | KRVILADANLDKVLSAYNKHRDKPIREQAENIIHLFTLTNLGAPAAFKYEDTTIDRKR | |
| | YTSTKEVLDATLIHQSITGLYETRIDLSQLGGD | |
| 7 | MDKKYSIGLDIGTNSVGWAVITDEYKVPSKKFKVLGNTDRHSIKKNLIGALLFDSGET | WT Sp Cas9 (aa) |
| | AEATRLKRTARRRYTRRKNRICYLQEIFSNEMAKVDDSFFHRLEESFLVEEDKKHERH | |
| | PIFGNIVDEVAYHEKYPTIYHLRKKLVDSTDKADLRLIYLALAHMIKERGHFLIEGDL | |
| | NPDNSDVDKLFIQLVQTYNQLFEENPINASGVDAKAILSARLSKSRRLENLIAQLPGE | |
| | KKNGLFGNLIALSLGLTPNFKSNFDLAEDAKLQLSKDTYDDDLDNLLAQIGDQYADLF | |
| | LAAKNLSDAILLSDILRVNTEITKAPLSASMIKRYDEHHQDLTLLKALVRQQLPEKYK | |
| | EIFFDQSKNGYAGYIDGGASQEEFYKFIKPILEKMDGTEELLVKLNREDLLRKQRTED | |
| | NGSIPHQIHLGELHAILRRQEDFYPFLKDNREKIEKILTFRIPYYVGPLARGNSRFAW | |
| | MTRKSEETITPWNFEEVVDKGASAQSFIERMTNFDKNLPNEKVLPKHSLLYEYFTVYN | |
| | ELTKVKYVTEGMRKPAFLSGEQKKAIVDLLFKTNRKVTVKQLKEDYFKKIECFDSVEI | |
| | SGVEDRENASLGTYHDLLKIIKDKDELDNEENEDILEDIVLTLTLFEDREMIEERLKT | |
| | YAHLFDDKVMKQLKRRRYTGWGRLSRKLINGIRDKQSGKTILDFLKSDGFANRNEMQL | |
| | IHDDSLTFKEDIQKAQVSGQGDSLHEHIANLAGSPAIKKGILQTVKVVDELVKVMGRH | |
| | KPENIVIEMARENQTTQKGQKNSRERMKRIEEGIKELGSQILKEHPVENTQLQNEKLY | |
| | LYYLQNGRDMYVDQELDINRLSDYDVDHIVPQSFLKDDSIDNKVLTRSDKNRGKSDNV | |
| | PSEEVVKKMKNYWRQLLNAKLITQRKFDNLTKAERGGLSELDKAGFIKRQLVETRQIT | |
| | KHVAQILDSRMNTKYDENDKLIREVKVITLKSKLVSDERKDFQFYKVREINNYHHAHD | |
| | AYLNAVVGTALIKKYPKLESEFVYGDYKVYDVRKMIAKSEQEIGKATAKYFFYSNIMN | |
| | FFKTEITLANGEIRKRPLIETNGETGEIVWDKGRDFATVRKVLSMPQVNIVKKTEVQT | |
| | GGFSKESILPKRNSDKLIARKKDWDPKKYGGFDSPTVAYSVLVVAKVEKGKSKKLKSV | |
| | KELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEF | |
| | SKRVILADANLDKVLSAYNKHRDKPIREQAENIIHLFTLTNLGAPAAFKYEDTTIDRK | |
| | RYTSTKEVLDATLIHQSITGLYETRIDLSQLGGD | |
| 8 | DKKYSIGLDIGTNSVGWAVITDEYKVPSKKFKVLGNTDRHSIKKNLIGALLFDSGETA | WT Sp Cas9 (aa) |
| | EATRLKRTARRRYTRRKNRICYLQEIFSNEMAKVDDSFFHRLEESFLVEEDKKHERHP | |
| | IFGNIVDEVAYHEKYPTIYHLRKKLVDSTDKADLRLIYLALAHMIKERGHFLIEGDLN | |
| | PDNSDVDKLFIQLVQTYNQLFEENPINASGVDAKAILSARLSKSRRLENLIAQLPGEK | |
| | KNGLFGNLIALSLGLTPNFKSNFDLAEDAKLQLSKDTYDDDLDNLLAQIGDQYADLFL | |
| | AAKNLSDAILLSDILRVNTEITKAPLSASMIKRYDEHHQDLTLLKALVRQQLPEKYKE | |
| | IFFDQSKNGYAGYIDGGASQEEFYKFIKPILEKMDGTEELLVKLNREDLLRKQRTFDN | |
| | GSIPHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTVYNE | |
| | LTKVKYVTEGMRKPAFLSGEQKKAIVDLLFKTNRKVTVKQLKEDYFKKIECFDSVEIS | |
| | GVEDRFNASLGTYHDLLKIIKDKDELDNEENEDILEDIVLTLTLFEDREMIEERLKTY | |
| | AHLFDDKVMKQLKRRRYTGWGRLSRKLINGIRDKQSGKTILDFLKSDGFANRNEMQLI | |
| | HDDSLTFKEDIQKAQVSGQGDSLHEHIANLAGSPAIKKGILQTVKVVDELVKVMGRHK | |
| | PENIVIEMARENQTTQKGQKNSRERMKRIEEGIKELGSQILKEHPVENTQLQNEKLYL | |
| | YYLQNGRDMYVDQELDINRLSDYDVDHIVPQSFLKDDSIDNKVLTRSDKNRGKSDNVP | |
| | SEEVVKKMKNYWRQLLNAKLITQRKFDNLTKAERGGLSELDKAGFIKRQLVETRQITK | |
| | HVAQILDSRMNTKYDENDKLIREVKVITLKSKLVSDFRKDFQFYKVREINNYHHAHDA | |
| | YLNAVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAGE | |
| | LQKGNELALPSKYVNFLYLASHYEKLKGSPEDNEQKQLFVEQHKHYLDEIIEQISEFS | |
| | KRVILADANLDKVLSAYNKHRDKPIREQAENIIHLFTLTNLGAPAAFKYFDTTIDRKR | |
| | YTSTKEVLDATLIHQSITGLYETRIDLSQLGGD | |
| 9 | AAGAGCAAGAAGGGCAGACAGGAGGCCCTGGAGAGACTGAAGAAGGCCAAGGCCGGCG | DPOLA (nt) |
| | AGAAGTACAAGTACGAGGTGGAGGACTTCACCGGCGTGTACGAGGAGGTGGACGAGGA | |
| | GCAGTACAGCAAGCTGGTGCAGGCCAGACAGGACGACGACTGGATCGTGGACGACGAC | |
| | GGCATCGGCTACGTGGAGGACGGCAGAGAGATCTTCGACGACGACCTGGAGGACGACG | |
| | CCCTGGAC | |
| 10 | AGCAAGCCCGAGAAGATCCTGAAGAAGGGCACCTACGACAAGGCCTACACCGACGAGC | ENL (nt) |
| | TGGTGGAGCTGCACAGAAGACTGATGGCCCTGAGAGAGAGAAACGTGCTGCAGCAGAT | |
| | CGTGAACCTGATCGAGGAGACCGGCCACTTCAACGTGACCAACACCACCTTCGACTTC | |
| | GACCTGTTCAGCCTGGACGAGACCACCGTGAGAAAGCTGCAGAGCTGCCTGGAGGCCG | |
| | TGGCCACC | |
| 11 | GACAGCCTGAGCGGCAGCAGCCTGTACAGCACCAGCGCCAACCTGCCCGTGATGGGCC | FOXO3 (nt) |
| | ACGAGAAGTTCCCCAGCGACCTGGACCTGGACATGTTCAACGGCAGCCTGGAGTGCGA | |
| | CATGGAGAGCATCATCAGAAGCGAGCTGATGGACGCCGACGGCCTGGACTTCAACTTC | |
| | GACAGCCTGATCAGCACCCAGAACGTGGTGGGCCTGAACGTGGGCAACTTCACCGGCG | |
| | CCAAGCAG | |
| 12 | CCCGAGTGGTTCCACGGCGCCATCAGCAGAGAGGACGCCGAGAACCTGCTGGAGAGCC | HSH2D (nt) |
| | AGCCCCTGGGCAGCTTCCTGATCAGAGTGAGCCACAGCCACGTGGGCTACACCCTGAG | |
| | CTACAAGGCCCAGAGCAGCTGCTGCCACTTCATGGTGAAGCTGCTGGACGACGGCACC | |
| | TTCATGATCCCCGGCGAGAAGGTGGCCCACACCAGCCTGGACGCCCTGGTGACCTTCC | |
| | ACCAGCAG | |
| 13 | CCCTTCGGCAGCAGCCCCGACGACCTGCTGTGCCCCCACCCCGCTGCTGAAAGCCCCA | NCOA2 (nt) |
| | GCGACGAGGGCGCTCTGCTGGACCAGCTGTACCTGGCCCTGAGAAACTTCGACGGCCT | |
| | GGAGGAGATCGACAGAGCCCTGGGCATCCCCGAGCTGGTGAGCCAGAGCCAGGCCGTG | |
| | GACCCCGAGCAGTTCAGCAGCCAGGACAGCAACATCATGCTGGAGCAGAAGGCCCCCG | |
| | TGTTCCCC | |
| 14 | CTGAGAAACAGCCTGGACGACCTGGTGGGCCCCCCCAGCAACCTGGAGGGCCAGAGCG | NCOA3 (nt) |
| | ACGAGAGAGCCCTGCTGGACCAGCTGCACACCCTGCTGAGCAACACCGACGCCACCGG | |
| | CCTGGAGGAGATCGACAGAGCCCTGGGCATCCCCGAGCTGGTGAACCAGGGCCAGGCC | |
| | CTGGAGCCCAAGCAGGACGCCTTCCAGGGCCAGGAGGCTGCCGTGATGATGGACCAGA | |
| | AGGCCGGC | |
| 15 | TTCAGAAACCAGTACGACAACGACGTGACCGTGTGGAGCCCCCAGGGCAGAATCCACC | PSA1 (nt) |
| | AGATCGAGTACGCCATGGAGGCCGTGAAGCAGGGCAGCGCCACCGTGGGCCTGAAGAG | |
| | CAAGACCCACGCCGTGCTGGTGGCCCTGAAGAGAGCCCAGAGCGAGCTGGCCGCCCAC | |
| | CAGAAGAAGATCCTGCACGTGGACAACCACATCGGCATCAGCATCGCCGGCCTGACCG | |
| | CCGACGCC | |
| 16 | AGACACGGCCACAGCAGCAGCGACCCCGTGTACCCCTGCGGCATCTGCACCAACGAGG | PYGO1 (nt) |
| | TGAACGACGACCAGGACGCCATCCTGTGCGAGGCCAGCTGCCAGAAGTGGTTCCACAG | |
| | AATCTGCACCGGCATGACCGAGACCGCCTACGGCCTGCTGACCGCCGAGGCCAGCGCT | |
| | GTGTGGGGATGCGACACCTGCATGGCCGACAAGGACGTGCAGCTGATGAGAACCAGAG | |
| | AGACCTTC | |
| 17 | GGCCCCATGAGACTGTACGTGGGCAGCCTGCACTTCAACATCACCGAGGACATGCTGA | RBM39 (nt) |
| | GAGGCATCTTCGAGCCCTTCGGCAGAATCGAGAGCATCCAGCTGATGATGGACAGCGA | |
| | GACCGGCAGAAGCAAGGGCTACGGCTTCATCACCTTCAGCGACAGCGAGTGCGCCAAG | |
| | AAGGCCCTGGAGCAGCTGAACGGCTTCGAGCTGGCCGGCAGACCCATGAAGGTGGGCC | |
| | ACGTGACC | |
| 18 | ACCCTGATCAGAAAGGCCGACCTGGAGAACCACAACAAGGACGGCGGCTTCTGGACCG | HERC2 (nt) |
| | TGATCGACGGCAAGGTGTACGACATCAAGGACTTCCAGACCCAGAGCCTGACCGGCAA | |
| | CAGCATCCTGGCCCAGTTCGCCGGCGAGGACCCCGTGGTGGCCCTGGAAGCTGCTCTG | |
| | CAGTTCGAGGACACCAGAGAGAGCATGCACGCCTTCTGCGTGGGCCAGTACCTGGAGC | |
| | CCGACCAG | |
| 19 | CCCTACTACATCAACCACGAGACCCAGACCACCTGCTGGGACCACCCCAAGATGACCG | DMD control (nt) |
| | AGCTGTACCAGAGCCTGGCCGACCTGAACAACGTGAGATTCAGCGCCTACAGGACCGC | |
| | CATGAAGCTGAGAAGACTGCAGAAGGCCCTGTGCCTGGACCTGCTGAGCCTGAGCGCC | |
| | GCCTGCGACGCCTTGGACCAGCACAACCTGAAGCAGAACGACCAGCCCATGGACATCC | |
| | TGCAGATC | |
| 20 | AACAGATACAGCTGCGTGTGCAGCCCCGGCTTCACCGGCCAGAGATGCAACATCGACA | NOTCH2 (nt) |
| | TCGACGAGTGCGCCAGCAACCCCTGCAGAAAGGGCGCCACCTGCATCAACGGCGTGAA | |
| | CGGCTTCAGATGCATCTGCCCCGAGGGCCCCCACCACCCCAGCTGCTACAGCCAGGTG | |
| | AACGAGTGCCTGAGCAACCCCTGCATCCACGGCAACTGCACCGGCGGCCTGAGCGGCT | |
| | ACAAGTGC | |
| 21 | GIVTLPHRYVYLIRFLADHVDASQSLTTRTRIDTFMCVACSFDGSNYTAVLSPQILAI | Control peptide |
| | RRNSCVRDMRFVARREYTKAVE | (aa) |
| 22 | KSKKGRQEALERLKKAKAGEKYKYEVEDFTGVYEEVDEEQYSKLVQARQDDDWIVDDD | DPOLA (aa) |
| | GIGYVEDGREIFDDDLEDDALD | |
| 23 | SKPEKILKKGTYDKAYTDELVELHRRLMALRERNVLQQIVNLIEETGHENVINTTEDE | ENL (aa) |
| | DLFSLDETTVRKLQSCLEAVAT | |
| 24 | DSLSGSSLYSTSANLPVMGHEKFPSDLDLDMENGSLECDMESIIRSELMDADGLDENF | FOXO3 (aa) |
| | DSLISTQNVVGLNVGNFTGAKQ | |
| 25 | PEWFHGAISREDAENLLESQPLGSFLIRVSHSHVGYTLSYKAQSSCCHFMVKLLDDGT | HSH2D (aa) |
| | FMIPGEKVAHTSLDALVTFHQQ | |
| 26 | PFGSSPDDLLCPHPAAESPSDEGALLDQLYLALRNEDGLEEIDRALGIPELVSQSQAV | NCOA2 (aa) |
| | DPEQFSSQDSNIMLEQKAPVFP | |
| 27 | LRNSLDDLVGPPSNLEGQSDERALLDQLHTLLSNTDATGLEEIDRALGIPELVNQGQA | NCOA3 (aa) |
| | LEPKQDAFQGQEAAVMMDQKAG | |
| 28 | FRNQYDNDVTVWSPQGRIHQIEYAMEAVKQGSATVGLKSKTHAVLVALKRAQSELAAH | PSA1 (aa) |
| | QKKILHVDNHIGISIAGLTADA | |
| 29 | RHGHSSSDPVYPCGICTNEVNDDQDAILCEASCQKWFHRICTGMTETAYGLLTAEASA | PYGO1 (aa) |
| | VWGCDTCMADKDVQLMRTRETF | |
| 30 | GPMRLYVGSLHFNITEDMLRGIFEPFGRIESIQLMMDSETGRSKGYGFITFSDSECAK | RBM39 (aa) |
| | KALEQLNGFELAGRPMKVGHVT | |
| 31 | TLIRKADLENHNKDGGFWTVIDGKVYDIKDFQTQSLTGNSILAQFAGEDPVVALEAAL | HERC2 (aa) |
| | QFEDTRESMHAFCVGQYLEPDQ | |
| 32 | PYYINHETQTTCWDHPKMTELYQSLADLNNVRESAYRTAMKLRRLQKALCLDLLSLSA | DMD (aa) |
| | ACDALDQHNLKQNDQPMDILQI | |
| 33 | NRYSCVCSPGFTGQRCNIDIDECASNPCRKGATCINGVNGFRCICPEGPHHPSCYSQV | NOTCH2 (aa) |
| | NECLSNPCIHGNCTGGLSGYKC | |
| 34 | IFKPEELRQALMPTLEALYRQDPESLPFRQPVDPQLLGIPDYFDIVKSPMDLSTIKRK | p300 core (aa) |
| | LDTGQYQEPWQYVDDIWLMENNAWLYNRKTSRVYKYCSKLSEVFEQEIDPVMQSLGYC | |
| | CGRKLEFSPQTLCCYGKQLCTIPRDATYYSYQNRYHFCEKCFNEIQGESVSLGDDPSQ | |
| | PQTTINKEQFSKRKNDTLDPELFVECTECGRKMHQICVLHHEIIWPAGFVCDGCLKKS | |
| | ARTRKENKFSAKRLPSTRLGTFLENRVNDFLRRQNHPESGEVTVRVVHASDKTVEVKP | |
| | GMKARFVDSGEMAESFPYRTKALFAFEEIDGVDLCFFGMHVQEYGSDCPPPNQRRVYI | |
| | SYLDSVHFFRPKCLRTAVYHEILIGYLEYVKKLGYTTGHIWACPPSEGDDYIFHCHPP | |
| | DQKIPKPKRLQEWYKKMLDKAVSERIVHDYKDIFKQATEDRLTSAKELPYFEGDFWPN | |
| | VLEESIKELEQEEEERKREENTSNESTDVTKGDSKNAKKKNNKKTSKNKSSLSRGNKK | |
| | KPGMPNVSNDLSQKLYATMEKHKEVFFVIRLIAGPAANSLPPIVDPDPLIPCDLMDGR | |
| | DAFLTLARDKHLEFSSLRRAQWSTMCMLVELHTQSQD | |
| 35 | MAPVHGDDCEIGASALSDSGSFVSSRARREKKSKKGRQEALERLKKAKAGEKYKYEVE | DNA polymerase |
| | DFTGVYEEVDEEQYSKLVQARQDDDWIVDDDGIGYVEDGREIFDDDLEDDALDADEKG | alpha catalytic |
| | KDGKARNKDKRNVKKLAVTKPNNIKSMFIACAGKKTADKAVDLSKDGLLGDILQDLNT | subunit (DPOLA) |
| | ETPQITPPPVMILKKKRSIGASPNPFSVHTATAVPSGKIASPVSRKEPPLTPVPLKRA | isoform 1 [Homo |
| | EFAGDDVQVESTEEEQESGAMEFEDGDFDEPMEVEEVDLEPMAAKAWDKESEPAEEVK | sapiens] (aa) |
| | QEADSGKGTVSYLGSFLPDVSCWDIDQEGDSSFSVQEVQVDSSHLPLVKGADEEQVFH | |
| | FYWLDAYEDQYNQPGVVFLFGKVWIESAETHVSCCVMVKNIERTLYFLPREMKIDLNT | |
| | GKETGTPISMKDVYEEFDEKIATKYKIMKFKSKPVEKNYAFEIPDVPEKSEYLEVKYS | |
| | AEMPQLPQDLKGETFSHVFGTNTSSLELFLMNRKIKGPCWLEVKSPQLLNQPVSWCKV | |
| | EAMALKPDLVNVIKDVSPPPLVVMAFSMKTMQNAKNHQNEIIAMAALVHHSFALDKAA | |
| | PKPPFQSHFCVVSKPKDCIFPYAFKEVIEKKNVKVEVAATERTLLGFFLAKVHKIDPD | |
| | IIVGHNIYGFELEVLLQRINVCKAPHWSKIGRLKRSNMPKLGGRSGFGERNATCGRMI | |
| | CDVEISAKELIRCKSYHLSELVQQILKTERVVIPMENIQNMYSESSQLLYLLEHTWKD | |
| | AKFILQIMCELNVLPLALQITNIAGNIMSRTLMGGRSERNEFLLLHAFYENNYIVPDK | |
| | QIFRKPQQKLGDEDEEIDGDTNKYKKGRKKAAYAGGLVLDPKVGFYDKFILLLDENSL | |
| | YPSIIQEFNICFTTVQRVASEAQKVTEDGEQEQIPELPDPSLEMGILPREIRKLVERR | |
| | KQVKQLMKQQDLNPDLILQYDIRQKALKLTANSMYGCLGFSYSRFYAKPLAALVTYKG | |
| | REILMHTKEMVQKMNLEVIYGDTDSIMINTNSTNLEEVFKLGNKVKSEVNKLYKLLEI | |
| | DIDGVFKSLLLLKKKKYAALVVEPTSDGNYVTKQELKGLDIVRRDWCDLAKDTGNFVI | |
| | GQILSDQSRDTIVENIQKRLIEIGENVLNGSVPVSQFEINKALTKDPQDYPDKKSLPH | |
| | VHVALWINSQGGRKVKAGDTVSYVICQDGSNLTASQRAYAPEQLQKQDNLTIDTQYYL | |
| | AQQIHPVVARICEPIDGIDAVLIATWLGLDPTQFRVHHYHKDEENDALLGGPAQLTDE | |
| | EKYRDCERFKCPCPTCGTENIYDNVEDGSGTDMEPSLYRCSNIDCKASPLTFTVQLSN | |
| | KLIMDIRRFIKKYYDGWLICEEPTCRNRTRHLPLQFSRTGPLCPACMKATLQPEYSDK | |
| | SLYTQLCFYRYIFDAECALEKLTTDHEKDKLKKQFFTPKVLQDYRKLKNTAEQFLSRS | |
| | GYSEVNLSKLFAGCAVKS | |
| 36 | MCAFPAFPQFWDSEIPGQRHQLRSSFCVCVCTCAPVCRLDSDFKRCTVQVRLELGHRA | protein ENL (ENL) |
| | QLRKKPTTEGFTHDWMVFVRGPEQCDIQHFVEKVVFWLHDSFPKPRRVCKEPPYKVEE | isoform X1 [Homo |
| | SGYAGFIMPIEVHFKNKEEPRKVCFTYDLFLNLEGNPPVNHLRCEKLTENNPTTEFRY | sapiens] (aa) |
| | KLLRAGGVMVMPEGADTVSRPSPDYPMLPTIPLSAFSDPKKTKPSHGSKDANKESSKT | |
| | SKPHKVTKEHRERPRKDSESKSSSKELEREQAKSSKDTSRKLGEGRLPKEEKAPPPKA | |
| | AFKEPKMALKETKLESTSPKGGPPPPPPPPPRASSKRPATADSPKPSAKKQKKSSSKG | |
| | SRSAPGTSPRTSSSSSFSDKKPAKDKSSTRGEKVKAESEPREAKKALEVEESNSEDEA | |
| | SFKSESAQSSPSNSSSSSDSSSDSDFEPSQNHSQGPLRSMVEDLQSEESDEDDSSSGE | |
| | EAAGKTNPGRDSRLSFSDSESDNSADSSLPSREPPPPQKPPPPNSKVSGRRSPESCSK | |
| | PEKILKKGTYDKAYTDELVELHRRLMALRERNVLQQIVNLIEETGHFNVTNTTFDEDL | |
| | FSLDETTVRKLQSCLEAVAT | |
| 37 | MAEAPASPAPLSPLEVELDPEFEPQSRPRSCTWPLQRPELQASPAKPSGETAADSMIP | forkhead box |
| | EEEDDEDDEDGGGRAGSAMAIGGGGGSGTLGSGLLLEDSARVLAPGGQDPGSGPATAA | protein O3 |
| | GGLSGGTQALLQPQQPLPPPQPGAAGGSGQPRKCSSRRNAWGNLSYADLITRAIESSP | (FOXO3) [Homo |
| | DKRLTLSQIYEWMVRCVPYFKDKGDSNSSAGWKNSIRHNLSLHSRFMRVQNEGTGKSS | sapiens] (aa) |
| | WWIINPDGGKSGKAPRRRAVSMDNSNKYTKSRGRAAKKKAALQTAPESADDSPSQLSK | |
| | WPGSPTSRSSDELDAWTDFRSRTNSNASTVSGRLSPIMASTELDEVQDDDAPLSPMLY | |
| | SSSASLSPSVSKPCTVELPRLTDMAGTMNLNDGLTENLMDDLLDNITLPPSQPSPTGG | |
| | LMQRSSSFPYTTKGSGLGSPTSSFNSTVFGPSSLNSLRQSPMQTIQENKPATFSSMSH | |
| | YGNQTLQDLLTSDSLSHSDVMMTQSDPLMSQASTAVSAQNSRRNVMLRNDPMMSFAAQ | |
| | PNQGSLVNQNLLHHQHQTQGALGGSRALSNSVSNMGLSESSSLGSAKHQQQSPVSQSM | |
| | QTLSDSLSGSSLYSTSANLPVMGHEKFPSDLDLDMENGSLECDMESIIRSELMDADGL | |
| | DFNFDSLISTQNVVGLNVGNFTGAKQASSQSWVPG | |
| 38 | MTEAGKLPLPLPPRLDWFVHTQMGQLAQDGVPEWFHGAISREDAENLLESQPLGSFLI | hematopoietic SH2 |
| | RVSHSHVGYTLSYKAQSSCCHFMVKLLDDGTFMIPGEKVAHTSLDALVTFHQQKPIEP | domain-containing |
| | RRELLTQPCRQKDPANVDYEDLFLYSNAVAEEAACPVSAPEEASPKPVLCHQSKERKP | protein (HSH2D) |
| | SAEMNRITTKEATSSCPPKSPLGETRQKLWRSLKMLPERGQRVRQQLKSHLATVNLSS | isoform 1 [Homo |
| | LLDVRRSTVISGPGTGKGSQDHSGDPTSGDRGYTDPCVATSLKSPSQPQAPKDRKVPT | sapiens] (aa) |
| | RKAERSVSCIEVTPGDRSWHQMVVRALSSQESKPEHQGLAEPENDQLPEEYQQPPPFA | |
| | PGYC | |
| 39 | MSGMGENTSDPSRAETRKRKECPDQLGPSPKRNTEKRNREQENKYIEELAELIFANEN | nuclear receptor |
| | DIDNFNFKPDKCAILKETVKQIRQIKEQEKAAAANIDEVQKSDVSSTGQGVIDKDALG | coactivator 2 |
| | PMMLEALDGFFFVVNLEGNVVFVSENVTQYLRYNQEELMNKSVYSILHVGDHTEFVKN | (NCOA2) isoform |
| | LLPKSIVNGGSWSGEPPRRNSHTFNCRMLVKPLPDSEEEGHDNQEAHQKYETMQCFAV | X1 [Homo sapiens] |
| | SQPKSIKEEGEDLQSCLICVARRVPMKERPVLPSSESFTTRQDLQGKITSLDTSTMRA | (aa) |
| | AMKPGWEDLVRRCIQKFHAQHEGESVSYAKRHHHEVLRQGLAFSQIYRFSLSDGTLVA | |
| | AQTKSKLIRSQTTNEPQLVISLHMLHREQNVCVMNPDLTGQTMGKPLNPISSNSPAHQ | |
| | ALCSGNPGQDMTLSSNINFPINGPKEQMGMPMGRFGGSGGMNHVSGMQATTPQGSNYA | |
| | LKMNSPSQSSPGMNPGQPTSMLSPRHRMSPGVAGSPRIPPSQFSPAGSLHSPVGVCSS | |
| | TGNSHSYTNSSLNALQALSEGHGVSLGSSLASPDLKMGNLQNSPVNMNPPPLSKMGSL | |
| | DSKDCFGLYGEPSEGTTGQAESSCHPGEQKETNDPNLPPAVSSERADGQSRLHDSKGQ | |
| | TKLLQLLTTKSDQMEPSPLASSLSDTNKDSTGSLPGSGSTHGTSLKEKHKILHRLLQD | |
| | SSSPVDLAKLTAEATGKDLSQESSSTAPGSEVTIKQEPVSPKKKENALLRYLLDKDDT | |
| | KDIGLPEITPKLERLDSKTDPASNTKLIAMKTEKEEMSFEPGDQPGSELDNLEEILDD | |
| | LQNSQLPQLFPDTRPGAPAGSVDKQAIINDLMQLTAENSPVTPVGAQKTALRISQSTF | |
| | NNPRPGQLGRLLPNQNLPLDITLQSPTGAGPFPPIRNSSPYSVIPQPGMMGNQGMIGN | |
| | QGNLGNSSTGMIGNSASRPTMPSGEWAPQSSAVRVTCAATTSAMNRPVQGGMIRNPAA | |
| | SIPMRPSSQPGQRQTLQSQVMNIGPSELEMNMGGPQYSQQQAPPNQTAPWPESILPID | |
| | QASFASQNRQPFGSSPDDLLCPHPAAESPSDEGALLDQLYLALRNEDGLEEIDRALGI | |
| | PELVSQSQAVDPEQFSSQDSNIMLEQKAPVFPQQYASQAQMAQGSYSPMQDPNFHTMG | |
| | QRPSYATLRMQPRPGLRPTGLVQNQPNQLRLQLQHRLQAQQNRQPLMNQISNVSNVNL | |
| | TLRPGVPTQAPINAQMLAQRQREILNQHLRQRQMHQQQQVQQRTLMMRGQGLNMTPSM | |
| | VAPSGMPATMSNPRIPQANAQQFPFPPNYGTGLRSPPPFTSPFSPVSPSVGSQLLSHS | |
| | SLHGSQMNLANQGMIGNLGGQLGPVRSPQVQHSTFQALSSGISQQPDPGFTGATTPQS | |
| | PLMSPRMAHTQSPMMQQSQANPAYQAPSDINGWAQGNMGGNSMFSQQSPPHFGQQANT | |
| | SMYSNNMNINVSMATNTGGMSSMNQMTGQISMTSVTSVPTSGLSSMGPEQVNDPALRG | |
| | GNLFPNQLPGMDMIKQEGDTTRKYC | |
| 40 | MSGLGENLDPLASDSRKRKLPCDTPGQGLTCSGEKRRREQESKYIEELAELISANLSD | nuclear receptor |
| | IDNFNVKPDKCAILKETVRQIRQIKEQGKTISNDDDVQKADVSSTGQGVIDKDSLGPL | coactivator 3 |
| | LLQALDGFLFVVNRDGNIVFVSENVTQYLQYKQEDLVNTSVYNILHEEDRKDFLKNLP | (NCOA3) isoform a |
| | KSTVNGVSWTNETQRQKSHTFNCRMLMKTPHDILEDINASPEMRQRYETMQCFALSQP | [Homo sapiens] |
| | RAMMEEGEDLQSCMICVARRITTGERTFPSNPESFITRHDLSGKVVNIDTNSLRSSMR | (aa) |
| | PGFEDIIRRCIQRFFSLNDGQSWSQKRHYQEAYLNGHAETPVYRESLADGTIVTAQTK | |
| | SKLFRNPVTNDRHGFVSTHFLQREQNGYRPNPNPVGQGIRPPMAGCNSSVGGMSMSPN | |
| | QGLQMPSSRAYGLADPSTTGQMSGARYGGSSNIASLTPGPGMQSPSSYQNNNYGLNMS | |
| | SPPHGSPGLAPNQQNIMISPRNRGSPKIASHQFSPVAGVHSPMASSGNTGNHSFSSSS | |
| | LSALQAISEGVGTSLLSTLSSPGPKLDNSPNMNITQPSKVSNQDSKSPLGFYCDQNPV | |
| | ESSMCQSNSRDHLSDKESKESSVEGAENQRGPLESKGHKKLLQLLTCSSDDRGHSSLT | |
| | NSPLDSSCKESSVSVTSPSGVSSSTSGGVSSTSNMHGSLLQEKHRILHKLLQNGNSPA | |
| | EVAKITAEATGKDTSSITSCGDGNVVKQEQLSPKKKENNALLRYLLDRDDPSDALSKE | |
| | LQPQVEGVDNKMSQCTSSTIPSSSQEKDPKIKTETSEEGSGDLDNLDAILGDLTSSDF | |
| | YNNSISSNGSHLGTKQQVFQGTNSLGLKSSQSVQSIRPPYNRAVSLDSPVSVGSSPPV | |
| | KNISAFPMLPKQPMLGGNPRMMDSQENYGSSMGGPNRNVTVTQTPSSGDWGLPNSKAG | |
| | RMEPMNSNSMGRPGGDYNTSLPRPALGGSIPTLPLRSNSIPGARPVLQQQQQMLQMRP | |
| | GEIPMGMGANPYGQAAASNQLGSWPDGMLSMEQVSHGTQNRPLLRNSLDDLVGPPSNL | |
| | EGQSDERALLDQLHTLLSNTDATGLEEIDRALGIPELVNQGQALEPKQDAFQGQEAAV | |
| | MMDQKAGLYGQTYPAQGPPMQGGFHLQGQSPSFNSMMNQMNQQGNFPLQGMHPRANIM | |
| | RPRTNTPKQLRMQLQQRLQGQQFLNQSRQALELKMENPTAGGAAVMRPMMQPQVSSQQ | |
| | GFLNAQMVAQRSRELLSHHFRQQRVAMMMQQQQQQQQQQQQQQQQQQQQQQQQQQQQQ | |
| | TQAFSPPPNVTASPSMDGLLAGPTMPQAPPQQFPYQPNYGMGQQPDPAFGRVSSPPNA | |
| | MMSSRMGPSQNPMMQHPQAASIYQSSEMKGWPSGNLARNSSFSQQQFAHQGNPAVYSM | |
| | VHMNGSSGHMGQMNMNPMPMSGMPMGPDQKYC | |
| 41 | MQLSKVKFRNQYDNDVTVWSPQGRIHQIEYAMEAVKQGSATVGLKSKTHAVLVALKRA | proteasome subunit |
| | QSELAAHQKKILHVDNHIGISIAGLTADARLLCNEMRQECLDSRFVEDRPLPVSRLVS | alpha type-1 |
| | LIGSKTQIPTQRYGRRPYGVGLLIAGYDDMGPHIFQTCPSANYFDCRAMSIGARSQSA | (PSA1) isoform 1 |
| | RTYLERHMSEFMECNLNELVKHGLRALRETLPAEQDLTTKNVSIGIVGKDLEFTIYDD | [Homo sapiens] |
| | DDVSPFLEGLEERPQRKAQPAQPADEPAEKADEPMEH | (aa) |
| 42 | MPAENSPAPAYKVSSHGGDSGLDGLGGPGVQLGSPDKKKRKANTQGPSFPPLSEYAPP | pygopus homolog 1 |
| | PNPNSDHLVAANPFDDNYNTISYKPLPSSNPYLGPGYPGFGGYSTFRMPPHVPPRMSS | (PYGO1) isoform 1 |
| | PYCGPYSLRNQPHPFPQNPLGMGFNRPHAFNFGPHDNSSFGNPSYNNALSQNVNMPNQ | [Homo sapiens] |
| | HFRQNPAENFSQIPPQNASQVSNPDLASNFVPGNNSNFTSPLESNHSFIPPPNTFGQA | (aa) |
| | KAPPPKQDFTQGATKNTNQNSSAHPPHLNMDDTVNQSNIELKNVNRNNAVNQENSRSS | |
| | STEATNNNPANGTQNKPRQPRGAADACTTEKSNKSSLHPNRHGHSSSDPVYPCGICTN | |
| | EVNDDQDAILCEASCQKWFHRICTGMTETAYGLLTAEASAVWGCDTCMADKDVQLMRT | |
| | RETFGPSAVGSDA | |
| 43 | MADDIDIEAMLEAPYKKDENKLSSANGHEERSKKRKKSKSRSRSHERKRSKSKERKRS | RNA-binding |
| | RDRERKKSKSRERKRSRSKERRRSRSRSRDRRFRGRYRSPYSGPKFNSAIRGKIGLPH | protein 39 |
| | SIKLSRRRSRSKSPFRKDKSPVREPIDNLTPEERDARTVFCMQLAARIRPRDLEEFFS | (RBM39) isoform a |
| | TVGKVRDVRMISDRNSRRSKGIAYVEFVDVSSVPLAIGLTGQRVLGVPIIVQASQAEK | [Homo sapiens] |
| | NRAAAMANNLQKGSAGPMRLYVGSLHFNITEDMLRGIFEPFGRIESIQLMMDSETGRS | (aa) |
| | KGYGFITFSDSECAKKALEQLNGFELAGRPMKVGHVTERTDASSASSFLDSDELERTG | |
| | IDLGTTGRLQLMARLAEGTGLQIPPAAQQALQMSGSLAFGAVAEFSFVIDLQTRLSQQ | |
| | TEASALAAAASVQPLATQCFQLSNMFNPQTEEEVGWDTEIKDDVIEECNKHGGVIHIY | |
| | VDKNSAQGNVYVKCPSIAAAIAAVNALHGRWFAGKMITAAYVPLPTYHNLFPDSMTAT | |
| | QLLVPSRR | |
| 44 | MPSESFCLAAQARLDSKWLKTDIQLAFTRDGLCGLWNEMVKDGEIVYTGTESTQNGEL | E3 ubiquitin- |
| | PPRKDDSVEPSGTKKEDLNDKEKKDEEETPAPIYRAKSILDSWVWGKQPDVNELKECL | protein |
| | SVLVKEQQALAVQSATTTLSALRLKQRLVILERYFIALNRTVFQENVKVKWKSSGISL | ligase HERC2 |
| | PPVDKKSSRPAGKGVEGLARVGSRAALSFAFAFLRRAWRSGEDADLCSELLQESLDAL | (HERC2) [Homo |
| | RALPEASLFDESTVSSVWLEVVERATRFLRSVVTGDVHGTPATKGPGSIPLQDQHLAL | sapiens] (aa) |
| | AILLELAVQRGTLSQMLSAILLLLQLWDSGAQETDNERSAQGTSAPLLPLLQRFQSII | |
| | CRKDAPHSEGDMHLLSGPLSPNESFLRYLTLPQDNELAIDLRQTAVVVMAHLDRLATP | |
| | CMPPLCSSPTSHKGSLQEVIGWGLIGWKYYANVIGPIQCEGLANLGVTQIACAEKREL | |
| | ILSRNGRVYTQAYNSDTLAPQLVQGLASRNIVKIAAHSDGHHYLALAATGEVYSWGCG | |
| | DGGRLGHGDTVPLEEPKVISAFSGKQAGKHVVHIACGSTYSAAITAEGELYTWGRGNY | |
| | GRLGHGSSEDEAIPMLVAGLKGLKVIDVACGSGDAQTLAVTENGQVWSWGDGDYGKLG | |
| | RGGSDGCKTPKLIEKLQDLDVVKVRCGSQFSIALTKDGQVYSWGKGDNQRLGHGTEEH | |
| | VRYPKLLEGLQGKKVIDVAAGSTHCLALTEDSEVHSWGSNDQCQHFDTLRVTKPEPAA | |
| | LPGLDTKHIVGIACGPAQSFAWSSCSEWSIGLRVPFVVDICSMTFEQLDLLLRQVSEG | |
| | MDGSADWPPPQEKECVAVATLNLLRLQLHAAISHQVDPEFLGLGLGSILLNSLKQTVV | |
| | TLASSAGVLSTVQSAAQAVLQSGWSVLLPTAEERARALSALLPCAVSGNEVNISPGRR | |
| | FMIDLLVGSLMADGGLESALHAAITAEIQDIEAKKEAQKEKEIDEQEANASTFHRSRT | |
| | PLDKDLINTGICESSGKQCLPLVQLIQQLLRNIASQTVARLKDVARRISSCLDFEQHS | |
| | RERSASLDLLLRFQRLLISKLYPGESIGQTSDISSPELMGVGSLLKKYTALLCTHIGD | |
| | ILPVAASIASTSWRHFAEVAYIVEGDFTGVLLPELVVSIVLLLSKNAGLMQEAGAVPL | |
| | LGGLLEHLDRENHLAPGKERDDHEELAWPGIMESFFTGQNCRNNEEVTLIRKADLENH | |
| | NKDGGFWTVIDGKVYDIKDFQTQSLTGNSILAQFAGEDPVVALEAALQFEDTRESMHA | |
| | FCVGQYLEPDQEIVTIPDLGSLSSPLIDTERNLGLLLGLHASYLAMSTPLSPVEIECA | |
| | KWLQSSIFSGGLQTSQIHYSYNEEKDEDHCSSPGGTPASKSRLCSHRRALGDHSQAFL | |
| | QAIADNNIQDHNVKDFLCQIERYCRQCHLTTPIMFPPEHPVEEVGRLLLCCLLKHEDL | |
| | GHVALSLVHAGALGIEQVKHRTLPKSVVDVCRVVYQAKCSLIKTHQEQGRSYKEVCAP | |
| | VIERLRFLFNELRPAVCNDLSIMSKFKLLSSLPRWRRIAQKIIRERRKKRVPKKPEST | |
| | DDEEKIGNEESDLEEACILPHSPINVDKRPIAIKSPKDKWQPLLSTVTGVHKYKWLKQ | |
| | NVQGLYPQSPLLSTIAEFALKEEPVDVEKMRKCLLKQLERAEVRLEGIDTILKLASKN | |
| | FLLPSVQYAMFCGWQRLIPEGIDIGEPLTDCLKDVDLIPPENRMLLEVTFGKLYAWAV | |
| | QNIRNVLMDASAKFKELGIQPVPLQTITNENPSGPSLGTIPQARFLLVMLSMLTLQHG | |
| | ANNLDLLLNSGMLALTQTALRLIGPSCDNVEEDMNASAQGASATVLEETRKETAPVQL | |
| | PVSGPELAAMMKIGTRVMRGVDWKWGDQDGPPPGLGRVIGELGEDGWIRVQWDTGSTN | |
| | SYRMGKEGKYDLKLAELPAAAQPSAEDSDTEDDSEAEQTERNIHPTAMMFTSTINLLQ | |
| | TLCLSAGVHAEIMQSEATKTLCGLLRMLVESGTTDKTSSPNRLVYREQHRSWCTLGFV | |
| | RSIALTPQVCGALSSPQWITLLMKVVEGHAPFTATSLQRQILAVHLLQAVLPSWDKTE | |
| | RARDMKCLVEKLFDFLGSLLTTCSSDVPLLRESTLRRRRVRPQASLTATHSSTLAEEV | |
| | VALLRTLHSLTQWNGLINKYINSQLRSITHSFVGRPSEGAQLEDYFPDSENPEVGGLM | |
| | AVLAVIGGIDGRLRLGGQVMHDEFGEGTVTRITPKGKITVQFSDMRTCRVCPLNQLKP | |
| | LPAVAFNVNNLPFTEPMLSVWAQLVNLAGSKLEKHKIKKSTKQAFAGQVDLDLLRCQQ | |
| | LKLYILKAGRALLSHQDKLRQILSQPAVQETGTVHTDDGAVVSPDLGDMSPEGPQPPM | |
| | ILLQQLLASATQPSPVKAIFDKQELEAAALAVCQCLAVESTHPSSPGFEDCSSSEATT | |
| | PVAVQHIRPARVKRRKQSPVPALPIVVQLMEMGFSRRNIEFALKSLTGASGNASSLPG | |
| | VEALVGWLLDHSDIQVTELSDADTVSDEYSDEEVVEDVDDAAYSMSTGAVVTESQTYK | |
| | KRADFLSNDDYAVYVRENIQVGMMVRCCRAYEEVCEGDVGKVIKLDRDGLHDLNVQCD | |
| | WQQKGGTYWVRYIHVELIGYPPPSSSSHIKIGDKVRVKASVTTPKYKWGSVTHQSVGV | |
| | VKAFSANGKDIIVDFPQQSHWTGLLSEMELVPSIHPGVTCDGCQMFPINGSRFKCRNC | |
| | DDFDFCETCFKTKKHNTRHTFGRINEPGQSAVFCGRSGKQLKRCHSSQPGMLLDSWSR | |
| | MVKSLNVSSSVNQASRLIDGSEPCWQSSGSQGKHWIRLEIFPDVLVHRLKMIVDPADS | |
| | SYMPSLVVVSGGNSLNNLIELKTININPSDTTVPLLNDCTEYHRYIEIAIKQCRSSGI | |
| | DCKIHGLILLGRIRAEEEDLAAVPFLASDNEEEEDEKGNSGSLIRKKAAGLESAATIR | |
| | TKVFVWGLNDKDQLGGLKGSKIKVPSFSETLSALNVVQVAGGSKSLFAVTVEGKVYAC | |
| | GEATNGRLGLGISSGTVPIPRQITALSSYVVKKVAVHSGGRHATALTVDGKVFSWGEG | |
| | DDGKLGHFSRMNCDKPRLIEALKTKRIRDIACGSSHSAALTSSGELYTWGLGEYGRLG | |
| | HGDNTTQLKPKMVKVLLGHRVIQVACGSRDAQTLALTDEGLVESWGDGDFGKLGRGGS | |
| | EGCNIPQNIERLNGQGVCQIECGAQFSLALTKSGVVWTWGKGDYFRLGHGSDVHVRKP | |
| | QVVEGLRGKKIVHVAVGALHCLAVTDSGQVYAWGDNDHGQQGNGTTTVNRKPTLVQGL | |
| | EGQKITRVACGSSHSVAWTTVDVATPSVHEPVLFQTARDPLGASYLGVPSDADSSAAS | |
| | NKISGASNSKPNRPSLAKILLSLDGNLAKQQALSHILTALQIMYARDAVVGALMPAAM | |
| | IAPVECPSFSSAAPSDASAMASPMNGEECMLAVDIEDRLSPNPWQEKREIVSSEDAVT | |
| | PSAVTPSAPSASARPFIPVTDDLGAASIIAETMTKTKEDVESQNKAAGPEPQALDEFT | |
| | SLLIADDTRVVVDLLKLSVCSRAGDRGRDVLSAVLSGMGTAYPQVADMLLELCVTELE | |
| | DVATDSQSGRLSSQPVVVESSHPYTDDTSTSGTVKIPGAEGLRVEFDRQCSTERRHDP | |
| | LTVMDGVNRIVSVRSGREWSDWSSELRIPGDELKWKFISDGSVNGWGWRFTVYPIMPA | |
| | AGPKELLSDRCVLSCPSMDLVTCLLDERLNLASNRSIVPRLAASLAACAQLSALAASH | |
| | RMWALQRLRKLLTTEFGQSININRLLGENDGETRALSFTGSALAALVKGLPEALQRQF | |
| | EYEDPIVRGGKQLLHSPFFKVLVALACDLELDTLPCCAETHKWAWFRRYCMASRVAVA | |
| | LDKRTPLPRLFLDEVAKKIRELMADSENMDVLHESHDIFKREQDEQLVQWMNRRPDDW | |
| | TLSAGGSGTIYGWGHNHRGQLGGIEGAKVKVPTPCEALATLRPVQLIGGEQTLFAVTA | |
| | DGKLYATGYGAGGRLGIGGTESVSTPTLLESIQHVFIKKVAVNSGGKHCLALSSEGEV | |
| | YSWGEAEDGKLGHGNRSPCDRPRVIESLRGIEVVDVAAGGAHSACVTAAGDLYTWGKG | |
| | RYGRLGHSDSEDQLKPKLVEALQGHRVVDIACGSGDAQTLCLTDDDTVWSWGDGDYGK | |
| | LGRGGSDGCKVPMKIDSLTGLGVVKVECGSQFSVALTKSGAVYTWGKGDYHRLGHGSD | |
| | DHVRRPRQVQGLQGKKVIAIATGSLHCVCCTEDGEVYTWGDNDEGQLGDGTTNAIQRP | |
| | RLVAALQGKKVNRVACGSAHTLAWSTSKPASAGKLPAQVPMEYNHLQEIPIIALRNRL | |
| | LLLHHLSELFCPCIPMFDLEGSLDETGLGPSVGFDTLRGILISQGKEAAFRKVVQATM | |
| | VRDRQHGPVVELNRIQVKRSRSKGGLAGPDGTKSVFGQMCAKMSSFGPDSLLLPHRVW | |
| | KVKFVGESVDDCGGGYSESIAEICEELQNGLTPLLIVTPNGRDESGANRDCYLLSPAA | |
| | RAPVHSSMFRFLGVLLGIAIRTGSPLSLNLAEPVWKQLAGMSLTIADLSEVDKDFIPG | |
| | LMYIRDNEATSEEFEAMSLPFTVPSASGQDIQLSSKHTHITLDNRAEYVRLAINYRLH | |
| | EFDEQVAAVREGMARVVPVPLLSLFTGYELETMVCGSPDIPLHLLKSVATYKGIEPSA | |
| | SLIQWFWEVMESFSNTERSLFLRFVWGRTRLPRTIADFRGRDFVIQVLDKYNPPDHFL | |
| | PESYTCFFLLKLPRYSCKQVLEEKLKYAIHFCKSIDTDDYARIALTGEPAADDSSDDS | |
| | DNEDVDSFASDSTQDYLTGH | |
| 45 | MLWWEEVEDCYEREDVQKKTFTKWVNAQFSKFGKQHIENLFSDLQDGRRLLDLLEGLT | dystrophin (DMD) |
| | GQKLPKEKGSTRVHALNNVNKALRVLQNNNVDLVNIGSTDIVDGNHKLTLGLIWNIIL | isoform X1 [Homo |
| | HWQVKNVMKNIMAGLQQTNSEKILLSWVRQSTRNYPQVNVINFTTSWSDGLALNALIH | sapiens] (aa) |
| | SHRPDLFDWNSVVCQQSATQRLEHAFNIARYQLGIEKLLDPEDVDTTYPDKKSILMYI | |
| | TSLFQVLPQQVSIEAIQEVEMLPRPPKVTKEEHFQLHHQMHYSQQITVSLAQGYERTS | |
| | SPKPRFKSYAYTQAAYVTTSDPTRSPFPSQHLEAPEDKSFGSSLMESEVNLDRYQTAL | |
| | EEVLSWLLSAEDTLQAQGEISNDVEVVKDQFHTHEGYMMDLTAHQGRVGNILQLGSKL | |
| | IGTGKLSEDEETEVQEQMNLLNSRWECLRVASMEKQSNLHRVLMDLQNQKLKELNDWL | |
| | TKTEERTRKMEEEPLGPDLEDLKRQVQQHKVLQEDLEQEQVRVNSLTHMVVVVDESSG | |
| | DHATAALEEQLKVLGDRWANICRWTEDRWVLLQDILLKWQRLTEEQCLFSAWLSEKED | |
| | AVNKIHTTGFKDQNEMLSSLQKLAVLKADLEKKKQSMGKLYSLKQDLLSTLKNKSVTQ | |
| | KTEAWLDNFARCWDNLVQKLEKSTAQISQAVTTTQPSLTQTTVMETVTTVTTREQILV | |
| | KHAQEELPPPPPQKKRQITVDSEIRKRLDVDITELHSWITRSEAVLQSPEFAIFRKEG | |
| | NFSDLKEKVNAIEREKAEKFRKLQDASRSAQALVEQMVNEGVNADSIKQASEQLNSRW | |
| | IEFCQLLSERLNWLEYQNNIIAFYNQLQQLEQMTTTAENWLKIQPTTPSEPTAIKSQL | |
| | KICKDEVNRLSDLQPQIERLKIQSIALKEKGQGPMFLDADFVAFTNHFKQVFSDVQAR | |
| | EKELQTIFDTLPPMRYQETMSAIRTWVQQSETKLSIPQLSVTDYEIMEQRLGELQALQ | |
| | SSLQEQQSGLYYLSTTVKEMSKKAPSEISRKYQSEFEEIEGRWKKLSSQLVEHCQKLE | |
| | EQMNKLRKIQNHIQTLKKWMAEVDVFLKEEWPALGDSEILKKQLKQCRLLVSDIQTIQ | |
| | PSLNSVNEGGQKIKNEAEPEFASRLETELKELNTQWDHMCQQVYARKEALKGGLEKTV | |
| | SLQKDLSEMHEWMTQAEEEYLERDFEYKTPDELQKAVEEMKRAKEEAQQKEAKVKLLT | |
| | ESVNSVIAQAPPVAQEALKKELETLTTNYQWLCTRLNGKCKTLEEVWACWHELLSYLE | |
| | KANKWLNEVEFKLKTTENIPGGAEEISEVLDSLENLMRHSEDNPNQIRILAQTLTDGG | |
| | VMDELINEELETFNSRWRELHEEAVRRQKLLEQSIQSAQETEKSLHLIQESLTFIDKQ | |
| | LAAYIADKVDAAQMPQEAQKIQSDLTSHEISLEEMKKHNQGKEAAQRVLSQIDVAQKK | |
| | LQDVSMKFRLFQKPANFEQRLQESKMILDEVKMHLPALETKSVEQEVVQSQLNHCVNL | |
| | YKSLSEVKSEVEMVIKTGRQIVQKKQTENPKELDERVTALKLHYNELGAKVTERKQQL | |
| | EKCLKLSRKMRKEMNVLTEWLAATDMELTKRSAVEGMPSNLDSEVAWGKATQKEIEKQ | |
| | KVHLKSITEVGEALKTVLGKKETLVEDKLSLLNSNWIAVTSRAEEWLNLLLEYQKHME | |
| | TFDQNVDHITKWIIQADTLLDESEKKKPQQKEDVLKRLKAELNDIRPKVDSTRDQAAN | |
| | LMANRGDHCRKLVEPQISELNHRFAAISHRIKTGKASIPLKELEQFNSDIQKLLEPLE | |
| | AEIQQGVNLKEEDENKDMNEDNEGTVKELLQRGDNLQQRITDERKREEIKIKQQLLQT | |
| | KHNALKDLRSQRRKKALEISHQWYQYKRQADDLLKCLDDIEKKLASLPEPRDERKIKE | |
| | IDRELQKKKEELNAVRRQAEGLSEDGAAMAVEPTQIQLSKRWREIESKFAQFRRLNFA | |
| | QIHTVREETMMVMTEDMPLEISYVPSTYLTEITHVSQALLEVEQLLNAPDLCAKDFED | |
| | LFKQEESLKNIKDSLQQSSGRIDIIHSKKTAALQSATPVERVKLQEALSQLDFQWEKV | |
| | NKMYKDRQGRFDRSVEKWRRFHYDIKIFNQWLTEAEQFLRKTQIPENWEHAKYKWYLK | |
| | ELQDGIGQRQTVVRTLNATGEEIIQQSSKTDASILQEKLGSLNLRWQEVCKQLSDRKK | |
| | RLEEQKNILSEFQRDLNEFVLWLEEADNIASIPLEPGKEQQLKEKLEQVKLLVEELPL | |
| | RQGILKQLNETGGPVLVSAPISPEEQDKLENKLKQTNLQWIKVSRALPEKQGEIEAQI | |
| | KDLGQLEKKLEDLEEQLNHLLLWLSPIRNQLEIYNQPNQEGPFDVKETEIAVQAKQPD | |
| | VEEILSKGQHLYKEKPATQPVKRKLEDLSSEWKAVNRLLQELRAKQPDLAPGLTTIGA | |
| | SPTQTVTLVTQPVVTKETAISKLEMPSSLMLEVPALADENRAWTELTDWLSLLDQVIK | |
| | SQRVMVGDLEDINEMIIKQKATMQDLEQRRPQLEELITAAQNLKNKTSNQEARTIITD | |
| | RIERIQNQWDEVQEHLQNRRQQLNEMLKDSTQWLEAKEEAEQVLGQARAKLESWKEGP | |
| | YTVDAIQKKITETKQLAKDLRQWQTNVDVANDLALKLLRDYSADDTRKVHMITENINA | |
| | SWRSIHKRVSEREAALEETHRLLQQFPLDLEKFLAWLTEAETTANVLQDATRKERLLE | |
| | DSKGVKELMKQWQDLQGEIEAHTDVYHNLDENSQKILRSLEGSDDAVLLQRRLDNMNF | |
| | KWSELRKKSLNIRSHLEASSDQWKRLHLSLQELLVWLQLKDDELSRQAPIGGDFPAVQ | |
| | KQNDVHRAFKRELKTKEPVIMSTLETVRIFLTEQPLEGLEKLYQEPRELPPEERAQNV | |
| | TRLLRKQAEEVNTEWEKLNLHSADWQRKIDETLERLRELQEATDELDLKLRQAEVIKG | |
| | SWQPVGDLLIDSLQDHLEKVKALRGEIAPLKENVSHVNDLARQLTTLGIQLSPYNLST | |
| | LEDLNTRWKLLQVAVEDRVRQLHEAHRDFGPASQHELSTSVQGPWERAISPNKVPYYI | |
| | NHETQTTCWDHPKMTELYQSLADLNNVRESAYRTAMKLRRLQKALCLDLLSLSAACDA | |
| | LDQHNLKQNDQPMDILQIINCLTTIYDRLEQEHNNLVNVPLCVDMCLNWLLNVYDTGR | |
| | TGRIRVLSFKTGIISLCKAHLEDKYRYLFKQVASSTGFCDQRRLGLLLHDSIQIPRQL | |
| | GEVASFGGSNIEPSVRSCFQFANNKPEIEAALFLDWMRLEPQSMVWLPVLHRVAAAET | |
| | AKHQAKCNICKECPIIGFRYRSLKHFNYDICQSCFFSGRVAKGHKMHYPMVEYCTPTT | |
| | SGEDVRDFAKVLKNKFRTKRYFAKHPRMGYLPVQTVLEGDNMETPVTLINFWPVDSAP | |
| | ASSPQLSHDDTHSRIEHYASRLAEMENSNGSYLNDSISPNESIDDEHLLIQHYCQSLN | |
| | QDSPLSQPRSPAQILISLESEERGELERILADLEEENRNLQAEYDRLKQQHEHKGLSP | |
| | LPSPPEMMPTSPQSPRDAELIAEAKLLRQHKGRLEARMQILEDHNKQLESQLHRLRQL | |
| | LEQPQAEAKVNGTTVSSPSTSLQRSDSSQPMLLRVVGSQTSDSMGEEDLLSPPQDTST | |
| | GLEEVMEQLNNSFPSSRGHNVGSLFHMADDLGRAMESLVSVMTDEEGAE | |
| 46 | MPALRPALLWALLALWLCCAAPAHALQCRDGYEPCVNEGMCVTYHNGTGYCKCPEGEL | NOTCH2 (NOTC2) |
| | GEYCQHRDPCEKNRCQNGGTCVAQAMLGKATCRCASGFTGEDCQYSTSHPCFVSRPCL | [Homo sapiens] |
| | NGGTCHMLSRDTYECTCQVGFTGKECQWTDACLSHPCANGSTCTTVANQFSCKCLTGF | (aa) |
| | TGQKCETDVNECDIPGHCQHGGTCLNLPGSYQCQCPQGFTGQYCDSLYVPCAPSPCVN | |
| | GGTCRQTGDFTFECNCLPGFEGSTCERNIDDCPNHRCQNGGVCVDGVNTYNCRCPPQW | |
| | TGQFCTEDVDECLLQPNACQNGGTCANRNGGYGCVCVNGWSGDDCSENIDDCAFASCT | |
| | PGSTCIDRVASFSCMCPEGKAGLLCHLDDACISNPCHKGALCDTNPLNGQYICTCPQG | |
| | YKGADCTEDVDECAMANSNPCEHAGKCVNTDGAFHCECLKGYAGPRCEMDINECHSDP | |
| | CQNDATCLDKIGGFTCLCMPGFKGVHCELEINECQSNPCVNNGQCVDKVNRFQCLCPP | |
| | GFTGPVCQIDIDDCSSTPCLNGAKCIDHPNGYECQCATGFTGVLCEENIDNCDPDPCH | |
| | HGQCQDGIDSYTCICNPGYMGAICSDQIDECYSSPCLNDGRCIDLVNGYQCNCQPGTS | |
| | GVNCEINFDDCASNPCIHGICMDGINRYSCVCSPGFTGQRCNIDIDECASNPCRKGAT | |
| | CINGVNGFRCICPEGPHHPSCYSQVNECLSNPCIHGNCTGGLSGYKCLCDAGWVGINC | |
| | EVDKNECLSNPCQNGGTCDNLVNGYRCTCKKGFKGYNCQVNIDECASNPCLNQGTCFD | |
| | DISGYTCHCVLPYTGKNCQTVLAPCSPNPCENAAVCKESPNFESYTCLCAPGWQGQRC | |
| | TIDIDECISKPCMNHGLCHNTQGSYMCECPPGFSGMDCEEDIDDCLANPCQNGGSCMD | |
| | GVNTFSCLCLPGFTGDKCQTDMNECLSEPCKNGGTCSDYVNSYTCKCQAGFDGVHCEN | |
| | NINECTESSCFNGGTCVDGINSFSCLCPVGFTGSFCLHEINECSSHPCLNEGTCVDGL | |
| | GTYRCSCPLGYTGKNCQTLVNLCSRSPCKNKGTCVQKKAESQCLCPSGWAGAYCDVPN | |
| | VSCDIAASRRGVLVEHLCQHSGVCINAGNTHYCQCPLGYTGSYCEEQLDECASNPCQH | |
| | GATCSDFIGGYRCECVPGYQGVNCEYEVDECQNQPCQNGGTCIDLVNHFKCSCPPGTR | |
| | GLLCEENIDDCARGPHCLNGGQCMDRIGGYSCRCLPGFAGERCEGDINECLSNPCSSE | |
| | GSLDCIQLTNDYLCVCRSAFTGRHCETFVDVCPQMPCLNGGTCAVASNMPDGFICRCP | |
| | PGFSGARCQSSCGQVKCRKGEQCVHTASGPRCFCPSPRDCESGCASSPCQHGGSCHPQ | |
| | RQPPYYSCQCAPPFSGSRCELYTAPPSTPPATCLSQYCADKARDGVCDEACNSHACQW | |
| | DGGDCSLTMENPWANCSSPLPCWDYINNQCDELCNTVECLFDNFECQGNSKTCKYDKY | |
| | CADHFKDNHCNQGCNSEECGWDGLDCAADQPENLAEGTLVIVVLMPPEQLLQDARSFL | |
| | RALGTLLHTNLRIKRDSQGELMVYPYYGEKSAAMKKQRMTRRSLPGEQEQEVAGSKVF | |
| | LEIDNRQCVQDSDHCFKNTDAAAALLASHAIQGTLSYPLVSVVSESLTPERTQLLYLL | |
| | AVAVVIILFIILLGVIMAKRKRKHGSLWLPEGFTLRRDASNHKRREPVGQDAVGLKNL | |
| | SVQVSEANLIGTGTSEHWVDDEGPQPKKVKAEDEALLSEEDDPIDRRPWTQQHLEAAD | |
| | IRRTPSLALTPPQAEQEVDVLDVNVRGPDGCTPLMLASLRGGSSDLSDEDEDAEDSSA | |
| | NIITDLVYQGASLQAQTDRTGEMALHLAARYSRADAAKRLLDAGADANAQDNMGRCPL | |
| | HAAVAADAQGVFQILIRNRVTDLDARMNDGTTPLILAARLAVEGMVAELINCQADVNA | |
| | VDDHGKSALHWAAAVNNVEATLLLLKNGANRDMQDNKEETPLFLAAREGSYEAAKILL | |
| | DHFANRDITDHMDRLPRDVARDRMHHDIVRLLDEYNVTPSPPGTVLTSALSPVICGPN | |
| | RSFLSLKHTPMGKKSRRPSAKSTMPTSLPNLAKEAKDAKGSRRKKSLSEKVQLSESSV | |
| | TLSPVDSLESPHTYVSDTTSSPMITSPGILQASPNPMLATAAPPAPVHAQHALSFSNL | |
| | HEMQPLAHGASTVLPSVSQLLSHHHIVSPGSGSAGSLSRLHPVPVPADWMNRMEVNET | |
| | QYNEMFGMVLAPAEGTHPGIAPQSRPPEGKHITTPREPLPPIVTFQLIPKGSIAQPAG | |
| | APQPQSTCPPAVAGPLPTMYQIPEMARLPSVAFPTAMMPQQDGQVAQTILPAYHPFPA | |
| | SVGKYPTPPSQHSYASSNAAERTPSHSGHLQGEHPYLTPSPESPDQWSSSSPHSASDW | |
| | SDVTTSPTPGGAGGGQRGPGTHMSEPPHNNMQVYA | |
| 47 | MAENVVEPGPPSAKRPKLSSPALSASASDGTDFGSLEDLEHDLPDELINSTELGLING | p300 (aa) |
| | GDINQLQTSLGMVQDAASKHKQLSELLRSGSSPNLNMGVGGPGQVMASQAQQSSPGLG | |
| | LINSMVKSPMTQAGLTSPNMGMGTSGPNQGPTQSTGMMNSPVNQPAMGMNTGMNAGMN | |
| | PGMLAAGNGQGIMPNQVMNGSIGAGRGRQNMQYPNPGMGSAGNLLTEPLQQGSPQMGG | |
| | QTGLRGPQPLKMGMMNNPNPYGSPYTQNPGQQIGASGLGLQIQTKTVLSNNLSPFAMD | |
| | KKAVPGGGMPNMGQQPAPQVQQPGLVTPVAQGMGSGAHTADPEKRKLIQQQLVLLLHA | |
| | HKCQRREQANGEVRQCNLPHCRTMKNVLNHMTHCQSGKSCQVAHCASSRQIISHWKNC | |
| | TRHDCPVCLPLKNAGDKRNQQPILTGAPVGLGNPSSLGVGQQSAPNLSTVSQIDPSSI | |
| | ERAYAALGLPYQVNQMPTQPQVQAKNQQNQQPGQSPQGMRPMSNMSASPMGVNGGVGV | |
| | QTPSLLSDSMLHSAINSQNPMMSENASVPSMGPMPTAAQPSTTGIRKQWHEDITQDLR | |
| | NHLVHKLVQAIFPTPDPAALKDRRMENLVAYARKVEGDMYESANNRAEYYHLLAEKIY | |
| | KIQKELEEKRRTRLQKQNMLPNAAGMVPVSMNPGPNMGQPQPGMTSNGPLPDPSMIRG | |
| | SVPNQMMPRITPQSGLNQFGQMSMAQPPIVPRQTPPLQHHGQLAQPGALNPPMGYGPR | |
| | MQQPSNQGQFLPQTQFPSQGMNVTNIPLAPSSGQAPVSQAQMSSSSCPVNSPIMPPGS | |
| | QGSHIHCPQLPQPALHQNSPSPVPSRTPTPHHTPPSIGAQQPPATTIPAPVPTPPAMP | |
| | PGPQSQALHPPPRQTPTPPTTQLPQQVQPSLPAAPSADQPQQQPRSQQSTAASVPTPT | |
| | APLLPPQPATPLSQPAVSIEGQVSNPPSTSSTEVNSQAIAEKQPSQEVKMEAKMEVDQ | |
| | PEPADTQPEDISESKVEDCKMESTETEERSTELKTEIKEEEDQPSTSATQSSPAPGQS | |
| | KKKIFKPEELRQALMPTLEALYRQDPESLPFRQPVDPQLLGIPDYFDIVKSPMDLSTI | |
| | KRKLDTGQYQEPWQYVDDIWLMENNAWLYNRKTSRVYKYCSKLSEVFEQEIDPVMQSL | |
| | GYCCGRKLEFSPQTLCCYGKQLCTIPRDATYYSYQNRYHFCEKCFNEIQGESVSLGDD | |
| | PSQPQTTINKEQFSKRKNDTLDPELFVECTECGRKMHQICVLHHEIIWPAGFVCDGCL | |
| | KKSARTRKENKFSAKRLPSTRLGTFLENRVNDFLRRQNHPESGEVTVRVVHASDKTVE | |
| | VKPGMKARFVDSGEMAESFPYRTKALFAFEEIDGVDLCFFGMHVQEYGSDCPPPNQRR | |
| | VYISYLDSVHFFRPKCLRTAVYHEILIGYLEYVKKLGYTTGHIWACPPSEGDDYIFHC | |
| | HPPDQKIPKPKRLQEWYKKMLDKAVSERIVHDYKDIFKQATEDRLTSAKELPYFEGDE | |
| | WPNVLEESIKELEQEEEERKREENTSNESTDVTKGDSKNAKKKNNKKTSKNKSSLSRG | |
| | NKKKPGMPNVSNDLSQKLYATMEKHKEVFFVIRLIAGPAANSLPPIVDPDPLIPCDLM | |
| | DGRDAFLTLARDKHLEFSSLRRAQWSTMCMLVELHTQSQDRFVYTCNECKHHVETRWH | |
| | CTVCEDYDLCITCYNTKNHDHKMEKLGLGLDDESNNQQAAATQSPGDSRRLSIQRCIQ | |
| | SLVHACQCRNANCSLPSCQKMKRVVQHTKGCKRKTNGGCPICKQLIALCCYHAKHCQE | |
| | NKCPVPFCLNIKQKLRQQQLQHRLQQAQMLRRRMASMQRTGVVGQQQGLPSPTPATPT | |
| | TPTGQQPTTPQTPQPTSQPQPTPPNSMPPYLPRTQAAGPVSQGKAAGQVTPPTPPQTA | |
| | QPPLPGPPPAAVEMAMQIQRAAETQRQMAHVQIFQRPIQHQMPPMTPMAPMGMNPPPM | |
| | TRGPSGHLEPGMGPTGMQQQPPWSQGGLPQPQQLQSGMPRPAMMSVAQHGQPLNMAPQ | |
| | PGLGQVGISPLKPGTVSQQALQNLLRTLRSPSSPLQQQQVLSILHANPQLLAAFIKQR | |
| | AAKYANSNPQPIPGQPGMPQGQPGLQPPTMPGQQGVHSNPAMQNMNPMQAGVQRAGLP | |
| | QQQPQQQLQPPMGGMSPQAQQMNMNHNTMPSQFRDILRRQQMMQQQQQQGAGPGIGPG | |
| | MANHNQFQQPQGVGYPPQQQQRMQHHMQQMQQGNMGQIGQLPQALGAEAGASLQAYQQ | |
| | RLLQQQMGSPVQPNPMSPQQHMLPNQAQSPHLQGQQIPNSLSNQVRSPQPVPSPRPQS | |
| | QPPHSSPSPRMQPQPSPHHVSPQTSSPHPGLVAAQANPMEQGHFASPDQNSMLSQLAS | |
| | NPGMANLHGASATDLGLSTDNSDLNSNLSQSTLDIH | |
| 48 | GCAACAAACTTCTCTCTGCTGAAACAAGCCGGAGATGTCGAAGAGAATCCTGGACCG | P2A sequence (nt) |
| 49 | ATNFSLLKQAGDVEENPGP | P2A sequence (aa) |
| 50 | NGG | PAM-SpCas9, F. |
| | | Novicida Cas9 |
| 51 | NNGRRT | PAM-SaCas9 |
| 52 | NNNNGATT | PAM-N. |
| | | meningitidis Cas9 |
| 53 | NNNNRYAC | PAM-C. jejuni |
| | | Cas9 |
| 54 | NNAGAAW | PAM-S. |
| | | thermophilus Cas9 |
| 55 | NAAAAC | PAM-T. denticola |
| | | Cas9 |
| 56 | TTTV | PAM- |
| | | Cas12a/Cpf1 Cas9 |
| 57 | NGAN | Variant PAM- |
| | | SpCas9 variant |
| 58 | NGNG | Variant PAM- |
| | | SpCas9 variant |
| 59 | NGAG | Variant PAM- |
| | | SpCas9 variant |
| 60 | NGCG | Variant PAM- |
| | | SpCas9 variant |
| 61 | LLPKNYHLENEVARLKKLVGER | SunTag GCN4 |
| | | peptide (aa) |
| 62 | GGSGG | GGSGG linker (aa) |
| 63 | GGGGG | GGGGG linker (aa) |
| 64 | GGAGG | GGAGG linker (aa) |
| 65 | GGGGSSS | GGGGSSS linker |
| | | (aa) |
| 66 | GGGGAAA | GGGGAAA linker |
| | | (aa) |
| 67 | GGGGS | (GGGGS)n linker |
| | | (aa), n is 1 to 10 |
| 68 | AAAAGGCCGGCGGCCACGAAAAAGGCCGGCCAGGCAAAAAAGAAAAAG | nucleoplasmin NLS |
| | | (nt) |
| 69 | KRPAATKKAGQAKKKK | nucleoplasmin NLS |
| | | (aa) |
| 70 | PKKKRKV | SV40 virus large T- |
| | | antigen NLS |
| 71 | PAAKRVKLD | c-myc NLS (aa) |
| 72 | RQRRNELKRSP | c-myc NLS (aa) |
| 73 | NQSSNFGPMKGGNFGGRSSGPYGGGGQYFAKPRNQGGY | hRNPA1 M9 NLS |
| | | (aa) |
| 74 | RMRIZFKNKGKDTAELRRRRVEVSVELRKAKKDEQILKRRNV | importin-alpha IBB |
| | | domain NLS (aa) |
| 75 | VSRKRPRP | myoma T protein |
| | | NLS (aa) |
| 76 | PPKKARED | myoma T protein |
| | | NLS (aa) |
| 77 | PQPKKKPL | human p53 NLS |
| | | (aa) |
| 78 | SALIKKKKKMAP | mouse c-abl IV |
| | | NLS (aa) |
| 79 | DRLRR | influenza virus NS1 |
| | | NLS (aa) |
| 80 | PKQKKRK | influenza virus NS1 |
| | | NLS (aa) |
| 81 | RKLKKKIKKL | Hepatitis virus delta |
| | | antigen NLS (aa) |
| 82 | REKKKFLKRR | mouse Mx1 protein |
| | | MNLS (aa) |
| 83 | KRKGDEVDGVDEVAKKKSKK | human poly(ADP- |
| | | ribose) polymerase |
| | | NLS (aa) |
| 84 | RKCLQAGMNLEARKTKK | steroid hormone |
| | | receptors (human) |
| | | glucocorticoid NLS |
| | | (aa) |
| 85 | GACAAGAAGTACTCCATTGGGCTCGCCATCGGCACAAACAGCGTCGGCTGGGCCGTCA | N term dSpCas9 N |
| | TTACGGACGAGTACAAGGTGCCGAGCAAAAAATTCAAAGTTCTGGGCAATACCGATCG | terminal fragement |
| | CCACAGCATAAAGAAGAACCTCATTGGCGCCCTCCTGTTCGACTCCGGGGAAACCGCC | (nt) |
| | GAAGCCACGCGGCTCAAAAGAACAGCACGGCGCAGATATACCCGCAGAAAGAATCGGA | |
| | TCTGCTACCtgcaGGAGATCTTTAGTAATGAGATGGCTAAGGTGGATGACTCTTTCTT | |
| | CCATAGGCTGGAGGAGTCCTTTTTGGTGGAGGAGGATAAAAAGCACGAGCGCCACCCA | |
| | ATCTTTGGCAATATCGTGGACGAGGTGGCGTACCATGAAAAGTACCCAACCATATATC | |
| | ATCTGAGGAAGAAGCTTGTAGACAGTACTGATAAGGCTGACTTGCGGTTGATCTATCT | |
| | CGCGCTGGCGCATATGATCAAATTTCGGGGACACTTCCTCATCGAGGGGGACCTGAAC | |
| | CCAGACAACAGCGATGTCGACAAACTCTTTATCCAACTGGTTCAGACTTACAATCAGC | |
| | TTTTCGAAGAGAACCCGATCAACGCATCCGGAGTTGACGCCAAAGCAATCCTGAGCGC | |
| | TAGGCTGTCCAAATCCCGGCGGCTCGAAAACCTCATCGCACAGCTCCCTGGGGAGAAG | |
| | AAGAACGGCCTGTTTGGTAATCTTATCGCCCTGTCACTCGGGCTGACCCCCAACTTTA | |
| | AATCTAACTTCGACCTGGCCGAAGATGCCAAGCTTCAACTGAGCAAAGACACCTACGA | |
| | TGATGATCTCGACAATCTGCTGGCCCAGATCGGCGACCAGTACGCAGACCTTTTTTTG | |
| | GCGGCAAAGAACCTGTCAGACGCCATTCTGCTGAGTGATATTCTGCGAGTGAACACGG | |
| | AGATCACCAAAGCTCCGCTGAGCGCTAGTATGATCAAGCGCTATGATGAGCACCACCA | |
| | AGACTTGACTTTGCTGAAGGCCCTTGTCAGACAGCAACTGCCTGAGAAGTACAAGGAA | |
| | ATTTTCTTCGATCAGTCTAAAAATGGCTACGCCGGATACATTGACGGCGGAGCAAGCC | |
| | AGGAGGAATTTTACAAATTTATTAAGCCCATCTTGGAAAAAATGGACGGCACCGAGGA | |
| | GCTGCTGGTAAAGCTTAACAGAGAAGATCTGTTGCGCAAACAGCGCACTTTCGACAAT | |
| | GGAAGCATCCCCCACCAGATTCACCTGGGCGAACTGCACGCTATCCTCAGGCGGCAAG | |
| | AGGATTTCTACCCCTTTTTGAAAGATAACAGGGAAAAGATTGAGAAAATCCTCACATT | |
| | TCGGATACCCTACTATGTAGGCCCCCTCGCCCGGGGAAATTCCAGATTCGCGTGGATG | |
| | ACTCGCAAATCAGAAGAGACCATCACTCCCTGGAACTTCGAGGAAGTCGTGGATAAGG | |
| | GGGCCTCTGCCCAGTCCTTCATCGAAAGGATGACTAACTTTGATAAAAATCTGCCTAA | |
| | CGAAAAGGTGCTTCCTAAACACTCTCTGCTGTACGAGTACTTCACAGTTTATAACGAG | |
| | CTCACCAAGGTCAAATACGTCACAGAAGGGATGAGAAAGCCAGCATTCCTGTCTGGAG | |
| | AGCAGAAGAAAGCTATCGTGGACCTCCTCTTCAAGACGAACCGGAAAGTTACCGTGAA | |
| | ACAGCTCAAAGAAGACTATTTCAAAAAGATTGAA | |
| 86 | DKKYSIGLAIGTNSVGWAVITDEYKVPSKKFKVLGNTDRHSIKKNLIGALLFDSGETA | N term dSpCas9 N |
| | EATRLKRTARRRYTRRKNRICYLQEIFSNEMAKVDDSFFHRLEESFLVEEDKKHERHP | terminal fragement |
| | IFGNIVDEVAYHEKYPTIYHLRKKLVDSTDKADLRLIYLALAHMIKERGHFLIEGDLN | |
| | PDNSDVDKLFIQLVQTYNQLFEENPINASGVDAKAILSARLSKSRRLENLIAQLPGEK | |
| | KNGLFGNLIALSLGLTPNFKSNFDLAEDAKLQLSKDTYDDDLDNLLAQIGDQYADLFL | |
| | AAKNLSDAILLSDILRVNTEITKAPLSASMIKRYDEHHQDLTLLKALVRQQLPEKYKE | |
| | IFFDQSKNGYAGYIDGGASQEEFYKFIKPILEKMDGTEELLVKLNREDLLRKQRTEDN | |
| | GSIPHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTVYNE | |
| | LTKVKYVTEGMRKPAFLSGEQKKAIVDLLFKTNRKVTVKQLKEDYFKKIE | |
| 87 | TGCCTGTCCTACGAGACAGAGATCCTGACAGTGGAGTATGGCCTGCTGCCAATCGGCA | N term Npu Intein |
| | AGATCGTGGAGAAGAGGATCGAGTGTACCGTGTACTCTGTGGATAACAATGGCAACAT | (nt) |
| | CTATACACAGCCCGTGGCACAGTGGCACGATAGGGGAGAGCAGGAGGTGTTCGAGTAT | |
| | TGCCTGGAGGACGGCAGCCTGATCAGGGCAACCAAGGACCACAAGTTCATGACAGTGG | |
| | ATGGCCAGATGCTGCCCATCGAC | |
| 88 | CLSYETEILTVEYGLLPIGKIVEKRIECTVYSVDNNGNIYTQPVAQWHDRGEQEVFEY | N term Npu Intein |
| | CLEDGSLIRATKDHKFMTVDGQMLPID | (aa) |
| 89 | ATGAAACGGACAGCCGACGGAAGCGAGTTCGAGTCACCAAAGAAGAAGCGGAAAGTCA | dSpCas9-573-C |
| | TCAAGATTGCTACACGGAAATACCTGGGAAAGCAGAACGTGTACGACATCGGCGTGGA | Intein (nt) |
| | GCGGGATCACAACTTCGCCCTGAAGAATGGCTTTATCGCCAGCAATTGTTTCGACTCT | |
| | GTTGAAATCAGCGGAGTGGAGGATCGCTTCAACGCATCCCTGGGAACGTATCACGATC | |
| | TCCTGAAAATCATTAAAGACAAGGACTTCCTGGACAATGAGGAGAACGAGGACATTCT | |
| | TGAGGACATTGTCCTCACCCTTACGTTGTTTGAAGATAGGGAGATGATTGAAGAACGC | |
| | TTGAAAACTTACGCTCATCTCTTCGACGACAAAGTCATGAAACAGCTCAAGAGGCGCC | |
| | GATATACAGGATGGGGGCGGCTGTCAAGAAAACTGATCAATGGgatcCGAGACAAGCA | |
| | GAGTGGAAAGACAATCCTGGATTTTCTTAAGTCCGATGGATTTGCCAACCGGAACTTC | |
| | ATGCAGTTGATCCATGATGACTCTCTCACCTTTAAGGAGGACATCCAGAAAGCACAAG | |
| | TTTCTGGCCAGGGGGACAGTCTTCACGAGCACATCGCTAATCTTGCAGGTAGCCCAGC | |
| | TATCAAAAAGGGAATACTGCAGACCGTTAAGGTCGTGGATGAACTCGTCAAAGTAATG | |
| | GGAAGGCATAAGCCCGAGAATATCGTTATCGAGATGGCCCGAGAGAACCAAACTACCC | |
| | AGAAGGGACAGAAGAACAGTAGGGAAAGGATGAAGAGGATTGAAGAGGGTATAAAAGA | |
| | ACTGGGGTCCCAAATCCTTAAGGAACACCCAGTTGAAAACACCCAGCTTCAGAATGAG | |
| | AAGCTCTACCTGTACTACCTGCAGAACGGCAGGGACATGTACGTGGATCAGGAACTGG | |
| | ACATCAATCGGCTCTCCGACTACGACGTGGATGCCATCGTGCCCCAGTCTTTTCTCAA | |
| | AGATGATTCTATTGATAATAAAGTGTTGACAAGATCCGATAAAAATAGAGGGAAGAGT | |
| | GATAACGTCCCCTCAGAAGAAGTTGTCAAGAAAATGAAAAATTATTGGCGGCAGCTGC | |
| | TGAACGCCAAACTGATCACACAACGGAAGTTCGATAATCTGACTAAGGCTGAACGAGG | |
| | TGGCCTGTCTGAGTTGGATAAAGCCGGCTTCATCAAAAGGCAGCTTGTTGAGACACGC | |
| | CAGATCACCAAgcacGTGGCCCAAATTCTCGATTCACGCATGAACACCAAGTACGATG | |
| | AAAATGACAAACTGATTCGAGAGGTGAAAGTTATTACTCTGAAGTCTAAGCTGGTCTC | |
| | AGATTTCAGAAAGGACTTTCAGTTTTATAAGGTGAGAGAGATCAACAATTACCACCAT | |
| | GCGCATGATGCCTACCTGAATGCAGTGGTAGGCACTGCACTTATCAAAAAATATCCCA | |
| | AGCTTGAATCTGAATTTGTTTACGGAGACTATAAAGTGTACGATGTTAGGAAAATGAT | |
| | CGCAAAGTCTGAGCAGGAAATAGGCAAGGCCACCGCTAAGTACTTCTTTTACAGCAAT | |
| | ATTATGAATTTTTTCAAGACCGAGATTACACTGGCCAATGGAGAGATTCGGAAGCGAC | |
| | CACTTATCGAAACAAACGGAGAAACAGGAGAAATCGTGTGGGACAAGGGTAGGGATTT | |
| | CGCGACAGTCCGGAAGGTCCTGTCCATGCCGCAGGTGAACATCGTTAAAAAGACCGAA | |
| | GTACAGACCGGAGGCTTCTCCAAGGAAAGTATCCTCCCGAAAAGGAACAGCGACAAGC | |
| | TGATCGCACGCAAAAAAGATTGGGACCCCAAGAAATACGGCGGATTCGATTCTCCTAC | |
| | AGTCGCTTACAGTGTACTGGTTGTGGCCAAAGTGGAGAAAGGGAAGTCTAAAAAACTC | |
| | AAAAGCGTCAAGGAACTGCTGGGCATCACAATCATGGAGCGATCAAGCTTCGAAAAAA | |
| | ACCCCATCGACTTTCTCGAGGCGAAAGGATATAAAGAGGTCAAAAAAGACCTCATCAT | |
| | TAAGCTTCCCAAGTACTCTCTCTTTGAGCTTGAAAACGGCCGGAAACGAATGCTCGCT | |
| | AGTGCGGGCGAGCTGCAGAAAGGTAACGAGCTGGCACTGCCCTCTAAATACGTTAATT | |
| | TCTTGTATCTGGCCAGCCACTATGAAAAGCTCAAAGGGTCTCCCGAAGATAATGAGCA | |
| | GAAGCAGCTGTTCGTGGAACAACACAAACACTACCTTGATGAGATCATCGAGCAAATA | |
| | AGCGAATTCTCCAAAAGAGTGATCCTCGCCGACGCTAACCTCGATAAGGTGCTTTCTG | |
| | CTTACAATAAGCACAGGGATAAGCCCATCAGGGAGCAGGCAGAAAACATTATCCACTT | |
| | GTTTACTCTGACCAACTTGGGCGCGCCTGCAGCCTTCAAGTACTTCGACACCACCATA | |
| | GACAGAAAGCGGTACACCTCTACAAAGGAGGTCCTGGACGCCACACTGATTCATCAGT | |
| | CAATTACGGGGCTCTATGAAACAAGAATCGACCTCTCTCAGCTCGGTGGAGACAAAAG | |
| | GCCGGCGGCCACGAAAAAGGCCGGCCAGGCAAAAAAGAAAAAGTGA | |
| 90 | MKRTADGSEFESPKKKRKVIKIATRKYLGKQNVYDIGVERDHNFALKNGFIASNCFDS | dSpCas9-573-C |
| | VEISGVEDRENASLGTYHDLLKIIKDKDELDNEENEDILEDIVLTLTLFEDREMIEER | Intein (aa) |
| | LKTYAHLFDDKVMKQLKRRRYTGWGRLSRKLINGIRDKQSGKTILDFLKSDGFANRNE | |
| | MQLIHDDSLTFKEDIQKAQVSGQGDSLHEHIANLAGSPAIKKGILQTVKVVDELVKVM | |
| | GRHKPENIVIEMARENQTTQKGQKNSRERMKRIEEGIKELGSQILKEHPVENTQLQNE | |
| | KLYLYYLQNGRDMYVDQELDINRLSDYDVDAIVPQSFLKDDSIDNKVLTRSDKNRGKS | |
| | DNVPSEEVVKKMKNYWRQLLNAKLITQRKEDNLTKAERGGLSELDKAGFIKRQLVETR | |
| | QITKHVAQILDSRMNTKYDENDKLIREVKVITLKSKLVSDFRKDFQFYKVREINNYHH | |
| | AHDAYLNAVVGTALIKKYPKLESEFVYGDYKVYDVRKMIAKSEQEIGKATAKYFFYSN | |
| | IMNFFKTEITLANGEIRKRPLIETNGETGEIVWDKGRDFATVRKVLSMPQVNIVKKTE | |
| | VQTGGFSKESILPKRNSDKLIARKKDWDPKKYGGFDSPTVAYSVLVVAKVEKGKSKKL | |
| | KSVKELLGITIMERSSFEKNPIDFLEAKGYKEVKKDLIIKLPKYSLFELENGRKRMLA | |
| | SAGELQKGNELALPSKYVNFLYLASHYEKLKGSPEDNEQKQLFVEQHKHYLDEIIEQI | |
| | SEFSKRVILADANLDKVLSAYNKHRDKPIREQAENIIHLFTLTNLGAPAAFKYFDTTI | |
| | DRKRYTSTKEVLDATLIHQSITGLYETRIDLSQLGGDKRPAATKKAGQAKKKK | |
| 91 | ATCAAGATTGCTACACGGAAATACCTGGGAAAGCAGAACGTGTACGACATCGGCGTGG | C term Npu Intein |
| | AGCGGGATCACAACTTCGCCCTGAAGAATGGCTTTATCGCCAGCAAT | (nt) |
| 92 | IKIATRKYLGKQNVYDIGVERDHNFALKNGFIASN | C term Npu Intein |
| | | (aa) |
| 93 | TGTTTCGACTCTGTTGAAATCAGCGGAGTGGAGGATCGCTTCAACGCATCCCTGGGAA | C term dSpCas9 C |
| | CGTATCACGATCTCCTGAAAATCATTAAAGACAAGGACTTCCTGGACAATGAGGAGAA | terminal fragment |
| | CGAGGACATTCTTGAGGACATTGTCCTCACCCTTACGTTGTTTGAAGATAGGGAGATG | (nt) |
| | ATTGAAGAACGCTTGAAAACTTACGCTCATCTCTTCGACGACAAAGTCATGAAACAGC | |
| | TCAAGAGGCGCCGATATACAGGATGGGGGGGGCTGTCAAGAAAACTGATCAATGGgat | |
| | CCGAGACAAGCAGAGTGGAAAGACAATCCTGGATTTTCTTAAGTCCGATGGATTTGCC | |
| | AACCGGAACTTCATGCAGTTGATCCATGATGACTCTCTCACCTTTAAGGAGGACATCC | |
| | AGAAAGCACAAGTTTCTGGCCAGGGGGACAGTCTTCACGAGCACATCGCTAATCTTGC | |
| | AGGTAGCCCAGCTATCAAAAAGGGAATACTGCAGACCGTTAAGGTCGTGGATGAACTC | |
| | GTCAAAGTAATGGGAAGGCATAAGCCCGAGAATATCGTTATCGAGATGGCCCGAGAGA | |
| | ACCAAACTACCCAGAAGGGACAGAAGAACAGTAGGGAAAGGATGAAGAGGATTGAAGA | |
| | GGGTATAAAAGAACTGGGGTCCCAAATCCTTAAGGAACACCCAGTTGAAAACACCCAG | |
| | CTTCAGAATGAGAAGCTCTACCTGTACTACCTGCAGAACGGCAGGGACATGTACGTGG | |
| | ATCAGGAACTGGACATCAATCGGCTCTCCGACTACGACGTGGATGCCATCGTGCCCCA | |
| | GTCTTTTCTCAAAGATGATTCTATTGATAATAAAGTGTTGACAAGATCCGATAAAAAT | |
| | AGAGGGAAGAGTGATAACGTCCCCTCAGAAGAAGTTGTCAAGAAAATGAAAAATTATT | |
| | GGCGGCAGCTGCTGAACGCCAAACTGATCACACAACGGAAGTTCGATAATCTGACTAA | |
| | GGCTGAACGAGGTGGCCTGTCTGAGTTGGATAAAGCCGGCTTCATCAAAAGGCAGCTT | |
| | GTTGAGACACGCCAGATCACCAAgcacGTGGCCCAAATTCTCGATTCACGCATGAACA | |
| | CCAAGTACGATGAAAATGACAAACTGATTCGAGAGGTGAAAGTTATTACTCTGAAGTC | |
| | TAAGCTGGTCTCAGATTTCAGAAAGGACTTTCAGTTTTATAAGGTGAGAGAGATCAAC | |
| | AATTACCACCATGCGCATGATGCCTACCTGAATGCAGTGGTAGGCACTGCACTTATCA | |
| | AAAAATATCCCAAGCTTGAATCTGAATTTGTTTACGGAGACTATAAAGTGTACGATGT | |
| | TAGGAAAATGATCGCAAAGTCTGAGCAGGAAATAGGCAAGGCCACCGCTAAGTACTTC | |
| | TTTTACAGCAATATTATGAATTTTTTCAAGACCGAGATTACACTGGCCAATGGAGAGA | |
| | TTCGGAAGCGACCACTTATCGAAACAAACGGAGAAACAGGAGAAATCGTGTGGGACAA | |
| | GGGTAGGGATTTCGCGACAGTCCGGAAGGTCCTGTCCATGCCGCAGGTGAACATCGTT | |
| | AAAAAGACCGAAGTACAGACCGGAGGCTTCTCCAAGGAAAGTATCCTCCCGAAAAGGA | |
| | ACAGCGACAAGCTGATCGCACGCAAAAAAGATTGGGACCCCAAGAAATACGGCGGATT | |
| | CGATTCTCCTACAGTCGCTTACAGTGTACTGGTTGTGGCCAAAGTGGAGAAAGGGAAG | |
| | TCTAAAAAACTCAAAAGCGTCAAGGAACTGCTGGGCATCACAATCATGGAGCGATCAA | |
| | GCTTCGAAAAAAACCCCATCGACTTTCTCGAGGCGAAAGGATATAAAGAGGTCAAAAA | |
| | AGACCTCATCATTAAGCTTCCCAAGTACTCTCTCTTTGAGCTTGAAAACGGCCGGAAA | |
| | CGAATGCTCGCTAGTGCGGGCGAGCTGCAGAAAGGTAACGAGCTGGCACTGCCCTCTA | |
| | AATACGTTAATTTCTTGTATCTGGCCAGCCACTATGAAAAGCTCAAAGGGTCTCCCGA | |
| | AGATAATGAGCAGAAGCAGCTGTTCGTGGAACAACACAAACACTACCTTGATGAGATC | |
| | ATCGAGCAAATAAGCGAATTCTCCAAAAGAGTGATCCTCGCCGACGCTAACCTCGATA | |
| | AGGTGCTTTCTGCTTACAATAAGCACAGGGATAAGCCCATCAGGGAGCAGGCAGAAAA | |
| | CATTATCCACTTGTTTACTCTGACCAACTTGGGCGCGCCTGCAGCCTTCAAGTACTTC | |
| | GACACCACCATAGACAGAAAGCGGTACACCTCTACAAAGGAGGTCCTGGACGCCACAC | |
| | TGATTCATCAGTCAATTACGGGGCTCTATGAAACAAGAATCGACCTCTCTCAGCTCGG | |
| | TGGAGAC | |
| 94 | CFDSVEISGVEDRFNASLGTYHDLLKIIKDKDFLDNEENEDILEDIVLTLTLFEDREM | C term dSpCas9 C |
| | IEERLKTYAHLFDDKVMKQLKRRRYTGWGRLSRKLINGIRDKQSGKTILDFLKSDGFA | terminal fragment |
| | NRNFMQLIHDDSLTFKEDIQKAQVSGQGDSLHEHIANLAGSPAIKKGILQTVKVVDEL | (aa) |
| | VKVMGRHKPENIVIEMARENQTTQKGQKNSRERMKRIEEGIKELGSQILKEHPVENTQ | |
| | LQNEKLYLYYLQNGRDMYVDQELDINRLSDYDVDAIVPQSFLKDDSIDNKVLTRSDKN | |
| | RGKSDNVPSEEVVKKMKNYWRQLLNAKLITQRKFDNLTKAERGGLSELDKAGFIKRQL | |
| | VETRQITKHVAQILDSRMNTKYDENDKLIREVKVITLKSKLVSDFRKDFQFYKVREIN | |
| | NYHHAHDAYLNAVVGTALIKKYPKLESEFVYGDYKVYDVRKMIAKSEQEIGKATAKYF | |
| | FYSNIMNFFKTEITLANGEIRKRPLIETNGETGEIVWDKGRDFATVRKVLSMPQVNIV | |
| | KKTEVQTGGFSKESILPKRNSDKLIARKKDWDPKKYGGFDSPTVAYSVLVVAKVEKGK | |
| | SKKLKSVKELLGITIMERSSFEKNPIDFLEAKGYKEVKKDLIIKLPKYSLFELENGRK | |
| | RMLASAGELQKGNELALPSKYVNFLYLASHYEKLKGSPEDNEQKQLFVEQHKHYLDEI | |
| | IEQISEFSKRVILADANLDKVLSAYNKHRDKPIREQAENIIHLFTLTNLGAPAAFKYF | |
| | DTTIDRKRYTSTKEVLDATLIHQSITGLYETRIDLSQLGGD | |
| 95 | GGAGGGCCGAGCTCTGGCGCACCCCCACCAAGTGGAGGGTCTCCTGCCGGGTCCCCAA | extended linker (nt) |
| | CATCTACTGAAGAAGGCACCAGCGAATCCGCAACGCCCGAGTCAGGCCCTGGTACCTC | |
| | CACAGAACCATCTGAAGGTAGTGCGCCTGGTTCCCCAGCTGGAAGCCCTACTTCCACC | |
| | GAAGAAGGCACGTCAACCGAACCAAGTGAAGGATCTGCCCCTGGGACCAGCACTGAAC | |
| | CATCTGAG | |
| 96 | GGPSSGAPPPSGGSPAGSPTSTEEGTSESATPESGPGTSTEPSEGSAPGSPAGSPTST | extended linker (aa) |
| | EEGTSTEPSEGSAPGTSTEPSE | |
| 97 | CCCTGCGGCATCTGCACCAACGAGGTGAACGACGACCAGGACGCCATCCTGTGCGAGG | PYGO1 |
| | CCAGCTGCCAGAAGTGGTTCCACAGAATCTGCACCGGCATGACCGAGACCGCCTACGG | transcriptional |
| | CCTGCTGACCGCCGAGGCCAGCGCTGTGTGGGGATGCGACACCTGCATGGCCGAC | activation domain |
| | | (nt) |
| 98 | GCCTACACCGACGAGCTGGTGGAGCTGCACAGAAGACTGATGGCCCTGAGAGAGAGAA | ENL transcriptional |
| | ACGTGCTGCAGCAGATCGTGAACCTGATCGAGGAGACCGGCCACTTCAACGTGACCAA | activation domain |
| | CACCACCTTCGACTTCGACCTGTTCAGCCTGGACGAGACCACCGTGAGAAAGCTGCAG | (nt) |
| | AGCTGCCTGGAG | |
| 99 | CACGAGAAATTCCCCTCCGATTTGGATCTGGATATGTTCAACGGGTCACTTGAATGTG | FOXO3 |
| | ACATGGAGAGCATTATACGCTCCGAACTTATGGACGCAGACGGTCTTGACTTTAACTT | transcriptional |
| | TGATTCCCTC | activation domain, |
| | | codon optimized |
| | | (nt) |
| 100 | CACGAGAAGTTCCCCAGCGACCTGGACCTGGACATGTTCAACGGCAGCCTGGAGTGCG | FOXO3 |
| | ACATGGAGAGCATCATCAGAAGCGAGCTGATGGACGCCGACGGCCTGGACTTCAACTT | transcriptional |
| | CGACAGCCTG | activation domain |
| | | (nt) |
| 101 | GAGGGCCAGAGCGACGAGAGAGCCCTGCTGGACCAGCTGCACACCCTGCTGAGCAACA | NCOA3 |
| | CCGACGCCACCGGCCTGGAGGAGATCGACAGAGCCCTGGGCATCCCCGAGCTGGTGAA | transcriptional |
| | CCAGGGCCAGGCCCTGGAGCCCAAGCAGGAC | activation domain |
| | | (nt) |
| 102 | GAAGGGCAAAGCGACGAGCGAGCACTCTTGGATCAGCTCCACACGCTGCTCTCAAACA | NCOA3 |
| | CTGATGCCACTGGCTTGGAAGAGATCGATCGAGCATTGGGAATCCCAGAGCTTGTAAA | transcriptional |
| | TCAAGGTCAGGCGCTGGAACCTAAACAGGAT | activation domain, |
| | | codon optimized |
| | | (nt) |
| 103 | TGGTTCCACGGCGCCATCAGCAGAGAGGACGCCGAGAACCTGCTGGAGAGCCAGCCCC | HSH2D |
| | TGGGCAGCTTCCTGATCAGAGTGAGCCACAGCCACGTGGGCTACACCCTGAGCTACAA | transcriptional |
| | GGCCCAGAGCAGCTGCTGCCACTTCATGGTGAAGCTGCTGGACGACGGCACCTTCATG | activation domain |
| | ATCCCCGGCGAGAAGGTGGCCCACACCAGCCTGGACGCCCTGGTGACCTTCCAC | (nt) |
| 104 | GAAAGCCCCAGCGACGAGGGCGCTCTGCTGGACCAGCTGTACCTGGCCCTGAGAAACT | NCOA2 |
| | TCGACGGCCTGGAGGAGATCGACAGAGCCCTGGGCATCCCCGAGCTGGTGAGCCAGAG | transcriptional |
| | CCAGGCCGTGGACCCCGAGCAGTTC | activation domain |
| | | (nt) |
| 105 | GACATCGACGAGTGCGCCAGCAACCCCTGCAGAAAGGGCGCCACCTGCATCAACGGCG | NOTCH2 |
| | TGAACGGCTTCAGATGCATCTGCCCCGAGGGCCCCCACCACCCCAGCTGCTAC | transcriptional |
| | | activation domain |
| | | (nt) |
| 106 | AACGGAGGTGGAGGCTCA | Multipartite linker 1 |
| | | (nt) |
| 107 | GGTGGAGGAGGCAGT | Multipartite linker 2 |
| | | (nt) |
| 108 | GGAGGAGGAGGTTCTGGGGGTGGAGGTAGCGGTGGTGGAGGTAGT | Multipartite linker 3 |
| | | (nt) |
| 109 | CCCTGCGGCATCTGCACCAACGAGGTGAACGACGACCAGGACGCCATCCTGTGCGAGG | Bipartite activator- |
| | CCAGCTGCCAGAAGTGGTTCCACAGAATCTGCACCGGCATGACCGAGACCGCCTACGG | PYGO1 NCOA3 |
| | CCTGCTGACCGCCGAGGCCAGCGCTGTGTGGGGATGCGACACCTGCATGGCCGACAAC | (nt) |
| | GGAGGTGGAGGCTCAGAGGGCCAGAGCGACGAGAGAGCCCTGCTGGACCAGCTGCACA | |
| | CCCTGCTGAGCAACACCGACGCCACCGGCCTGGAGGAGATCGACAGAGCCCTGGGCAT | |
| | CCCCGAGCTGGTGAACCAGGGCCAGGCCCTGGAGCCCAAGCAGGAC | |
| 110 | GACATCGACGAGTGCGCCAGCAACCCCTGCAGAAAGGGCGCCACCTGCATCAACGGCG | Bipartite activator- |
| | TGAACGGCTTCAGATGCATCTGCCCCGAGGGCCCCCACCACCCCAGCTGCTACAACGG | NOTCH2 NCOA3 |
| | AGGTGGAGGCTCAGAGGGCCAGAGCGACGAGAGAGCCCTGCTGGACCAGCTGCACACC | (nt) |
| | CTGCTGAGCAACACCGACGCCACCGGCCTGGAGGAGATCGACAGAGCCCTGGGCATCC | |
| | CCGAGCTGGTGAACCAGGGCCAGGCCCTGGAGCCCAAGCAGGAC | |
| 111 | GAAGGGCAAAGCGACGAGCGAGCACTCTTGGATCAGCTCCACACGCTGCTCTCAAACA | Bipartite activator- |
| | CTGATGCCACTGGCTTGGAAGAGATCGATCGAGCATTGGGAATCCCAGAGCTTGTAAA | NCOA3 NCOA3 |
| | TCAAGGTCAGGCGCTGGAACCTAAACAGGATGGTGGAGGAGGCAGTGAGGGCCAGAGC | (nt) |
| | GACGAGAGAGCCCTGCTGGACCAGCTGCACACCCTGCTGAGCAACACCGACGCCACCG | |
| | GCCTGGAGGAGATCGACAGAGCCCTGGGCATCCCCGAGCTGGTGAACCAGGGCCAGGC | |
| | CCTGGAGCCCAAGCAGGAC | |
| 112 | TGGTTCCACGGCGCCATCAGCAGAGAGGACGCCGAGAACCTGCTGGAGAGCCAGCCCC | Bipartite activator- |
| | TGGGCAGCTTCCTGATCAGAGTGAGCCACAGCCACGTGGGCTACACCCTGAGCTACAA | HSH2D NCOA3 |
| | GGCCCAGAGCAGCTGCTGCCACTTCATGGTGAAGCTGCTGGACGACGGCACCTTCATG | (nt) |
| | ATCCCCGGCGAGAAGGTGGCCCACACCAGCCTGGACGCCCTGGTGACCTTCCACAACG | |
| | GAGGTGGAGGCTCAGAGGGCCAGAGCGACGAGAGAGCCCTGCTGGACCAGCTGCACAC | |
| | CCTGCTGAGCAACACCGACGCCACCGGCCTGGAGGAGATCGACAGAGCCCTGGGCATC | |
| | CCCGAGCTGGTGAACCAGGGCCAGGCCCTGGAGCCCAAGCAGGAC | |
| 113 | CACGAGAAGTTCCCCAGCGACCTGGACCTGGACATGTTCAACGGCAGCCTGGAGTGCG | Bipartite activator- |
| | ACATGGAGAGCATCATCAGAAGCGAGCTGATGGACGCCGACGGCCTGGACTTCAACTT | FOXO3 NCOA3 |
| | CGACAGCCTGGGTGGAGGAGGCAGTGAGGGCCAGAGCGACGAGAGAGCCCTGCTGGAC | (nt) |
| | CAGCTGCACACCCTGCTGAGCAACACCGACGCCACCGGCCTGGAGGAGATCGACAGAG | |
| | CCCTGGGCATCCCCGAGCTGGTGAACCAGGGCCAGGCCCTGGAGCCCAAGCAGGAC | |
| 114 | GAAAGCCCCAGCGACGAGGGCGCTCTGCTGGACCAGCTGTACCTGGCCCTGAGAAACT | Bipartite activator- |
| | TCGACGGCCTGGAGGAGATCGACAGAGCCCTGGGCATCCCCGAGCTGGTGAGCCAGAG | NCOA2 NCOA3 |
| | CCAGGCCGTGGACCCCGAGCAGTTCAACGGAGGTGGAGGCTCAGAGGGCCAGAGCGAC | (nt) |
| | GAGAGAGCCCTGCTGGACCAGCTGCACACCCTGCTGAGCAACACCGACGCCACCGGCC | |
| | TGGAGGAGATCGACAGAGCCCTGGGCATCCCCGAGCTGGTGAACCAGGGCCAGGCCCT | |
| | GGAGCCCAAGCAGGAC | |
| 115 | GCCTACACCGACGAGCTGGTGGAGCTGCACAGAAGACTGATGGCCCTGAGAGAGAGAA | Bipartite activator- |
| | ACGTGCTGCAGCAGATCGTGAACCTGATCGAGGAGACCGGCCACTTCAACGTGACCAA | ENL NCOA3 (nt) |
| | CACCACCTTCGACTTCGACCTGTTCAGCCTGGACGAGACCACCGTGAGAAAGCTGCAG | |
| | AGCTGCCTGGAGAACGGAGGTGGAGGCTCAGAGGGCCAGAGCGACGAGAGAGCCCTGC | |
| | TGGACCAGCTGCACACCCTGCTGAGCAACACCGACGCCACCGGCCTGGAGGAGATCGA | |
| | CAGAGCCCTGGGCATCCCCGAGCTGGTGAACCAGGGCCAGGCCCTGGAGCCCAAGCAG | |
| | GAC | |
| 116 | CCCTGCGGCATCTGCACCAACGAGGTGAACGACGACCAGGACGCCATCCTGTGCGAGG | Bipartite activator- |
| | CCAGCTGCCAGAAGTGGTTCCACAGAATCTGCACCGGCATGACCGAGACCGCCTACGG | PYGO1 FOXO3 |
| | CCTGCTGACCGCCGAGGCCAGCGCTGTGTGGGGATGCGACACCTGCATGGCCGACAAC | (nt) |
| | GGAGGTGGAGGCTCACACGAGAAGTTCCCCAGCGACCTGGACCTGGACATGTTCAACG | |
| | GCAGCCTGGAGTGCGACATGGAGAGCATCATCAGAAGCGAGCTGATGGACGCCGACGG | |
| | CCTGGACTTCAACTTCGACAGCCTG | |
| 117 | GACATCGACGAGTGCGCCAGCAACCCCTGCAGAAAGGGCGCCACCTGCATCAACGGCG | Bipartite activator- |
| | TGAACGGCTTCAGATGCATCTGCCCCGAGGGCCCCCACCACCCCAGCTGCTACAACGG | NOTCH2 FOXO3 |
| | AGGTGGAGGCTCACACGAGAAGTTCCCCAGCGACCTGGACCTGGACATGTTCAACGGC | (nt) |
| | AGCCTGGAGTGCGACATGGAGAGCATCATCAGAAGCGAGCTGATGGACGCCGACGGCC | |
| | TGGACTTCAACTTCGACAGCCTG | |
| 118 | GAAGGGCAAAGCGACGAGCGAGCACTCTTGGATCAGCTCCACACGCTGCTCTCAAACA | Bipartite activator- |
| | CTGATGCCACTGGCTTGGAAGAGATCGATCGAGCATTGGGAATCCCAGAGCTTGTAAA | NCOA3 FOXO3 |
| | TCAAGGTCAGGCGCTGGAACCTAAACAGGATAACGGAGGTGGAGGCTCACACGAGAAG | (nt) |
| | TTCCCCAGCGACCTGGACCTGGACATGTTCAACGGCAGCCTGGAGTGCGACATGGAGA | |
| | GCATCATCAGAAGCGAGCTGATGGACGCCGACGGCCTGGACTTCAACTTCGACAGCCT | |
| | G | |
| 119 | TGGTTCCACGGCGCCATCAGCAGAGAGGACGCCGAGAACCTGCTGGAGAGCCAGCCCC | Bipartite activator- |
| | TGGGCAGCTTCCTGATCAGAGTGAGCCACAGCCACGTGGGCTACACCCTGAGCTACAA | HSH2D FOXO3 |
| | GGCCCAGAGCAGCTGCTGCCACTTCATGGTGAAGCTGCTGGACGACGGCACCTTCATG | (nt) |
| | ATCCCCGGCGAGAAGGTGGCCCACACCAGCCTGGACGCCCTGGTGACCTTCCACAACG | |
| | GAGGTGGAGGCTCACACGAGAAGTTCCCCAGCGACCTGGACCTGGACATGTTCAACGG | |
| | CAGCCTGGAGTGCGACATGGAGAGCATCATCAGAAGCGAGCTGATGGACGCCGACGGC | |
| | CTGGACTTCAACTTCGACAGCCTG | |
| 120 | CACGAGAAATTCCCCTCCGATTTGGATCTGGATATGTTCAACGGGTCACTTGAATGTG | Bipartite activator- |
| | ACATGGAGAGCATTATACGCTCCGAACTTATGGACGCAGACGGTCTTGACTTTAACTT | FOXO3 FOXO3 |
| | TGATTCCCTCAACGGAGGTGGAGGCTCACACGAGAAGTTCCCCAGCGACCTGGACCTG | (nt) |
| | GACATGTTCAACGGCAGCCTGGAGTGCGACATGGAGAGCATCATCAGAAGCGAGCTGA | |
| | TGGACGCCGACGGCCTGGACTTCAACTTCGACAGCCTG | |
| 121 | GAAAGCCCCAGCGACGAGGGCGCTCTGCTGGACCAGCTGTACCTGGCCCTGAGAAACT | Bipartite activator- |
| | TCGACGGCCTGGAGGAGATCGACAGAGCCCTGGGCATCCCCGAGCTGGTGAGCCAGAG | NCOA2 FOXO3 |
| | CCAGGCCGTGGACCCCGAGCAGTTCAACGGAGGTGGAGGCTCACACGAGAAGTTCCCC | (nt) |
| | AGCGACCTGGACCTGGACATGTTCAACGGCAGCCTGGAGTGCGACATGGAGAGCATCA | |
| | TCAGAAGCGAGCTGATGGACGCCGACGGCCTGGACTTCAACTTCGACAGCCTG | |
| 122 | GCCTACACCGACGAGCTGGTGGAGCTGCACAGAAGACTGATGGCCCTGAGAGAGAGAA | Bipartite activator- |
| | ACGTGCTGCAGCAGATCGTGAACCTGATCGAGGAGACCGGCCACTTCAACGTGACCAA | ENL FOXO3 (nt) |
| | CACCACCTTCGACTTCGACCTGTTCAGCCTGGACGAGACCACCGTGAGAAAGCTGCAG | |
| | AGCTGCCTGGAGAACGGAGGTGGAGGCTCACACGAGAAGTTCCCCAGCGACCTGGACC | |
| | TGGACATGTTCAACGGCAGCCTGGAGTGCGACATGGAGAGCATCATCAGAAGCGAGCT | |
| | GATGGACGCCGACGGCCTGGACTTCAACTTCGACAGCCTG | |
| 123 | CCCTGCGGCATCTGCACCAACGAGGTGAACGACGACCAGGACGCCATCCTGTGCGAGG | Tripartite activator- |
| | CCAGCTGCCAGAAGTGGTTCCACAGAATCTGCACCGGCATGACCGAGACCGCCTACGG | PYGO1 FOXO3 |
| | CCTGCTGACCGCCGAGGCCAGCGCTGTGTGGGGATGCGACACCTGCATGGCCGACAAC | NCOA3 (nt) |
| | GGAGGTGGAGGCTCACACGAGAAGTTCCCCAGCGACCTGGACCTGGACATGTTCAACG | |
| | GCAGCCTGGAGTGCGACATGGAGAGCATCATCAGAAGCGAGCTGATGGACGCCGACGG | |
| | CCTGGACTTCAACTTCGACAGCCTGGGTGGAGGAGGCAGTGAGGGCCAGAGCGACGAG | |
| | AGAGCCCTGCTGGACCAGCTGCACACCCTGCTGAGCAACACCGACGCCACCGGCCTGG | |
| | AGGAGATCGACAGAGCCCTGGGCATCCCCGAGCTGGTGAACCAGGGCCAGGCCCTGGA | |
| | GCCCAAGCAGGAC | |
| 124 | GACATCGACGAGTGCGCCAGCAACCCCTGCAGAAAGGGCGCCACCTGCATCAACGGCG | Tripartite activator- |
| | TGAACGGCTTCAGATGCATCTGCCCCGAGGGCCCCCACCACCCCAGCTGCTACAACGG | NOTCH2 FOXO3 |
| | AGGTGGAGGCTCACACGAGAAGTTCCCCAGCGACCTGGACCTGGACATGTTCAACGGC | NCOA3 (nt) |
| | AGCCTGGAGTGCGACATGGAGAGCATCATCAGAAGCGAGCTGATGGACGCCGACGGCC | |
| | TGGACTTCAACTTCGACAGCCTGGGTGGAGGAGGCAGTGAGGGCCAGAGCGACGAGAG | |
| | AGCCCTGCTGGACCAGCTGCACACCCTGCTGAGCAACACCGACGCCACCGGCCTGGAG | |
| | GAGATCGACAGAGCCCTGGGCATCCCCGAGCTGGTGAACCAGGGCCAGGCCCTGGAGC | |
| | CCAAGCAGGAC | |
| 125 | GAAGGGCAAAGCGACGAGCGAGCACTCTTGGATCAGCTCCACACGCTGCTCTCAAACA | Tripartite activator- |
| | CTGATGCCACTGGCTTGGAAGAGATCGATCGAGCATTGGGAATCCCAGAGCTTGTAAA | NCOA3 FOXO3 |
| | TCAAGGTCAGGCGCTGGAACCTAAACAGGATAACGGAGGTGGAGGCTCACACGAGAAG | NCOA3 (nt) |
| | TTCCCCAGCGACCTGGACCTGGACATGTTCAACGGCAGCCTGGAGTGCGACATGGAGA | |
| | GCATCATCAGAAGCGAGCTGATGGACGCCGACGGCCTGGACTTCAACTTCGACAGCCT | |
| | GGGTGGAGGAGGCAGTGAGGGCCAGAGCGACGAGAGAGCCCTGCTGGACCAGCTGCAC | |
| | ACCCTGCTGAGCAACACCGACGCCACCGGCCTGGAGGAGATCGACAGAGCCCTGGGCA | |
| | TCCCCGAGCTGGTGAACCAGGGCCAGGCCCTGGAGCCCAAGCAGGAC | |
| 126 | TGGTTCCACGGCGCCATCAGCAGAGAGGACGCCGAGAACCTGCTGGAGAGCCAGCCCC | Tripartite activator- |
| | TGGGCAGCTTCCTGATCAGAGTGAGCCACAGCCACGTGGGCTACACCCTGAGCTACAA | HSH2D FOXO3 |
| | GGCCCAGAGCAGCTGCTGCCACTTCATGGTGAAGCTGCTGGACGACGGCACCTTCATG | NCOA3 (nt) |
| | ATCCCCGGCGAGAAGGTGGCCCACACCAGCCTGGACGCCCTGGTGACCTTCCACAACG | |
| | GAGGTGGAGGCTCACACGAGAAGTTCCCCAGCGACCTGGACCTGGACATGTTCAACGG | |
| | CAGCCTGGAGTGCGACATGGAGAGCATCATCAGAAGCGAGCTGATGGACGCCGACGGC | |
| | CTGGACTTCAACTTCGACAGCCTGGGTGGAGGAGGCAGTGAGGGCCAGAGCGACGAGA | |
| | GAGCCCTGCTGGACCAGCTGCACACCCTGCTGAGCAACACCGACGCCACCGGCCTGGA | |
| | GGAGATCGACAGAGCCCTGGGCATCCCCGAGCTGGTGAACCAGGGCCAGGCCCTGGAG | |
| | CCCAAGCAGGAC | |
| 127 | CACGAGAAATTCCCCTCCGATTTGGATCTGGATATGTTCAACGGGTCACTTGAATGTG | Tripartite activator- |
| | ACATGGAGAGCATTATACGCTCCGAACTTATGGACGCAGACGGTCTTGACTTTAACTT | FOXO3 FOXO3 |
| | TGATTCCCTCAACGGAGGTGGAGGCTCACACGAGAAGTTCCCCAGCGACCTGGACCTG | NCOA3 (nt) |
| | GACATGTTCAACGGCAGCCTGGAGTGCGACATGGAGAGCATCATCAGAAGCGAGCTGA | |
| | TGGACGCCGACGGCCTGGACTTCAACTTCGACAGCCTGGGTGGAGGAGGCAGTGAGGG | |
| | CCAGAGCGACGAGAGAGCCCTGCTGGACCAGCTGCACACCCTGCTGAGCAACACCGAC | |
| | GCCACCGGCCTGGAGGAGATCGACAGAGCCCTGGGCATCCCCGAGCTGGTGAACCAGG | |
| | GCCAGGCCCTGGAGCCCAAGCAGGAC | |
| 128 | GAAAGCCCCAGCGACGAGGGCGCTCTGCTGGACCAGCTGTACCTGGCCCTGAGAAACT | Tripartite activator- |
| | TCGACGGCCTGGAGGAGATCGACAGAGCCCTGGGCATCCCCGAGCTGGTGAGCCAGAG | NCOA2 FOXO3 |
| | CCAGGCCGTGGACCCCGAGCAGTTCAACGGAGGTGGAGGCTCACACGAGAAGTTCCCC | NCOA3 (nt) |
| | AGCGACCTGGACCTGGACATGTTCAACGGCAGCCTGGAGTGCGACATGGAGAGCATCA | |
| | TCAGAAGCGAGCTGATGGACGCCGACGGCCTGGACTTCAACTTCGACAGCCTGGGTGG | |
| | AGGAGGCAGTGAGGGCCAGAGCGACGAGAGAGCCCTGCTGGACCAGCTGCACACCCTG | |
| | CTGAGCAACACCGACGCCACCGGCCTGGAGGAGATCGACAGAGCCCTGGGCATCCCCG | |
| | AGCTGGTGAACCAGGGCCAGGCCCTGGAGCCCAAGCAGGAC | |
| 129 | GCCTACACCGACGAGCTGGTGGAGCTGCACAGAAGACTGATGGCCCTGAGAGAGAGAA | Tripartite activator- |
| | ACGTGCTGCAGCAGATCGTGAACCTGATCGAGGAGACCGGCCACTTCAACGTGACCAA | ENL FOXO3 |
| | CACCACCTTCGACTTCGACCTGTTCAGCCTGGACGAGACCACCGTGAGAAAGCTGCAG | NCOA3 (nt) |
| | AGCTGCCTGGAGAACGGAGGTGGAGGCTCACACGAGAAGTTCCCCAGCGACCTGGACC | |
| | TGGACATGTTCAACGGCAGCCTGGAGTGCGACATGGAGAGCATCATCAGAAGCGAGCT | |
| | GATGGACGCCGACGGCCTGGACTTCAACTTCGACAGCCTGGGTGGAGGAGGCAGTGAG | |
| | GGCCAGAGCGACGAGAGAGCCCTGCTGGACCAGCTGCACACCCTGCTGAGCAACACCG | |
| | ACGCCACCGGCCTGGAGGAGATCGACAGAGCCCTGGGCATCCCCGAGCTGGTGAACCA | |
| | GGGCCAGGCCCTGGAGCCCAAGCAGGAC | |
| 130 | PCGICTNEVNDDQDAILCEASCQKWFHRICTGMTETAYGLLTAEASAVWGCDTCMAD | PYGO1 |
| | | transcriptional |
| | | activation domain |
| | | (aa) |
| 131 | AYTDELVELHRRLMALRERNVLQQIVNLIEETGHFNVTNTTFDFDLESLDETTVRKLQ | ENL transcriptional |
| | SCLE | activation domain |
| | | (aa) |
| 132 | HEKFPSDLDLDMENGSLECDMESIIRSELMDADGLDENEDSL | FOXO3 |
| | | transcriptional |
| | | activation domain |
| | | (aa) |
| 133 | EGQSDERALLDQLHTLLSNTDATGLEEIDRALGIPELVNQGQALEPKQD | NCOA3 |
| | | transcriptional |
| | | activation domain |
| | | (aa) |
| 134 | WFHGAISREDAENLLESQPLGSFLIRVSHSHVGYTLSYKAQSSCCHFMVKLLDDGTFM | HSH2D |
| | IPGEKVAHTSLDALVTFH | transcriptional |
| | | activation domain |
| | | (aa) |
| 135 | ESPSDEGALLDQLYLALRNEDGLEEIDRALGIPELVSQSQAVDPEQF | NCOA2 |
| | | transcriptional |
| | | activation domain |
| | | (aa) |
| 136 | DIDECASNPCRKGATCINGVNGFRCICPEGPHHPSCY | NOTCH2 |
| | | transcriptional |
| | | activation domain |
| | | (aa) |
| 137 | NGGGGS | Multipartite linker 1 |
| | | (aa) |
| 138 | GGGGS | Multipartite linker 2 |
| | | (aa) |
| 139 | GGGGSGGGGSGGGGS | Multipartite linker 3 |
| | | (aa) |
| 140 | PCGICTNEVNDDQDAILCEASCQKWFHRICTGMTETAYGLLTAEASAVWGCDTCMADN | Bipartite activator- |
| | GGGGSEGQSDERALLDQLHTLLSNTDATGLEEIDRALGIPELVNQGQALEPKQD | PYGO1 NCOA3 |
| | | (aa) |
| 141 | DIDECASNPCRKGATCINGVNGFRCICPEGPHHPSCYNGGGGSEGQSDERALLDQLHT | Bipartite activator- |
| | LLSNTDATGLEEIDRALGIPELVNQGQALEPKQD | NOTCH2 NCOA3 |
| | | (aa) |
| 142 | EGQSDERALLDQLHTLLSNTDATGLEEIDRALGIPELVNQGQALEPKQDGGGGSEGQS | Bipartite activator- |
| | DERALLDQLHTLLSNTDATGLEEIDRALGIPELVNQGQALEPKQD | NCOA3 NCOA3 |
| | | (aa) |
| 143 | WFHGAISREDAENLLESQPLGSFLIRVSHSHVGYTLSYKAQSSCCHFMVKLLDDGTEM | Bipartite activator- |
| | IPGEKVAHTSLDALVTFHNGGGGSEGQSDERALLDQLHTLLSNTDATGLEEIDRALGI | HSH2D NCOA3 |
| | PELVNQGQALEPKQD | (aa) |
| 144 | HEKFPSDLDLDMENGSLECDMESIIRSELMDADGLDFNFDSLGGGGSEGQSDERALLD | Bipartite activator- |
| | QLHTLLSNTDATGLEEIDRALGIPELVNQGQALEPKQD | FOXO3 NCOA3 |
| | | (aa) |
| 145 | ESPSDEGALLDQLYLALRNEDGLEEIDRALGIPELVSQSQAVDPEQFNGGGGSEGQSD | Bipartite activator- |
| | ERALLDQLHTLLSNTDATGLEEIDRALGIPELVNQGQALEPKQD | NCOA2 NCOA3 |
| | | (aa) |
| 146 | AYTDELVELHRRLMALRERNVLQQIVNLIEETGHFNVTNTTFDFDLFSLDETTVRKLQ | Bipartite activator- |
| | SCLENGGGGSEGQSDERALLDQLHTLLSNTDATGLEEIDRALGIPELVNQGQALEPKQ | ENL NCOA3 (aa) |
| | D | |
| 147 | PCGICTNEVNDDQDAILCEASCQKWFHRICTGMTETAYGLLTAEASAVWGCDTCMADN | Bipartite activator- |
| | GGGGSHEKFPSDLDLDMENGSLECDMESIIRSELMDADGLDENEDSL | PYGO1 FOXO3 |
| | | (aa) |
| 148 | DIDECASNPCRKGATCINGVNGFRCICPEGPHHPSCYNGGGGSHEKFPSDLDLDMENG | Bipartite activator- |
| | SLECDMESIIRSELMDADGLDFNFDSL | NOTCH2 FOXO3 |
| | | (aa) |
| 149 | EGQSDERALLDQLHTLLSNTDATGLEEIDRALGIPELVNQGQALEPKQDNGGGGSHEK | Bipartite activator- |
| | FPSDLDLDMENGSLECDMESIIRSELMDADGLDENEDSL | NCOA3 FOXO3 |
| | | (aa) |
| 150 | WFHGAISREDAENLLESQPLGSFLIRVSHSHVGYTLSYKAQSSCCHFMVKLLDDGTEM | Bipartite activator- |
| | IPGEKVAHTSLDALVTFHNGGGGSHEKFPSDLDLDMENGSLECDMESIIRSELMDADG | HSH2D FOXO3 |
| | LDFNFDSL | (aa) |
| 151 | HEKFPSDLDLDMENGSLECDMESIIRSELMDADGLDENEDSLNGGGGSHEKFPSDLDL | Bipartite activator- |
| | DMFNGSLECDMESIIRSELMDADGLDENFDSL | FOXO3 FOXO3 |
| | | (aa) |
| 152 | ESPSDEGALLDQLYLALRNEDGLEEIDRALGIPELVSQSQAVDPEQENGGGGSHEKFP | Bipartite activator- |
| | SDLDLDMENGSLECDMESIIRSELMDADGLDENEDSL | NCOA2 FOXO3 |
| | | (aa) |
| 153 | AYTDELVELHRRLMALRERNVLQQIVNLIEETGHFNVTNTTFDFDLFSLDETTVRKLQ | Bipartite activator- |
| | SCLENGGGGSHEKFPSDLDLDMENGSLECDMESIIRSELMDADGLDFNFDSL | ENL FOXO3 (aa) |
| 154 | PCGICTNEVNDDQDAILCEASCQKWFHRICTGMTETAYGLLTAEASAVWGCDTCMADN | Tripartite activator- |
| | GGGGSHEKFPSDLDLDMENGSLECDMESIIRSELMDADGLDFNFDSLGGGGSEGQSDE | PYGO1 FOXO3 |
| | RALLDQLHTLLSNTDATGLEEIDRALGIPELVNQGQALEPKQD | NCOA3 (aa) |
| 155 | DIDECASNPCRKGATCINGVNGFRCICPEGPHHPSCYNGGGGSHEKFPSDLDLDMENG | Tripartite activator- |
| | SLECDMESIIRSELMDADGLDENFDSLGGGGSEGQSDERALLDQLHTLLSNTDATGLE | NOTCH2 FOXO3 |
| | EIDRALGIPELVNQGQALEPKQD | NCOA3 (aa) |
| 156 | EGQSDERALLDQLHTLLSNTDATGLEEIDRALGIPELVNQGQALEPKQDNGGGGSHEK | Tripartite activator- |
| | FPSDLDLDMENGSLECDMESIIRSELMDADGLDFNFDSLGGGGSEGQSDERALLDQLH | NCOA3 FOXO3 |
| | TLLSNTDATGLEEIDRALGIPELVNQGQALEPKQD | NCOA3 (aa) |
| 157 | WFHGAISREDAENLLESQPLGSFLIRVSHSHVGYTLSYKAQSSCCHFMVKLLDDGTFM | Tripartite activator- |
| | IPGEKVAHTSLDALVTFHNGGGGSHEKFPSDLDLDMENGSLECDMESIIRSELMDADG | HSH2D FOXO3 |
| | LDFNFDSLGGGGSEGQSDERALLDQLHTLLSNTDATGLEEIDRALGIPELVNQGQALE | NCOA3 (aa) |
| | PKQD | |
| 158 | HEKFPSDLDLDMENGSLECDMESIIRSELMDADGLDENFDSLNGGGGSHEKFPSDLDL | Tripartite activator- |
| | DMFNGSLECDMESIIRSELMDADGLDFNFDSLGGGGSEGQSDERALLDQLHTLLSNTD | FOXO3 FOXO3 |
| | ATGLEEIDRALGIPELVNQGQALEPKQD | NCOA3 (aa) |
| 159 | ESPSDEGALLDQLYLALRNEDGLEEIDRALGIPELVSQSQAVDPEQFNGGGGSHEKFP | Tripartite activator- |
| | SDLDLDMFNGSLECDMESIIRSELMDADGLDFNFDSLGGGGSEGQSDERALLDQLHTL | NCOA2 FOX03 |
| | LSNTDATGLEEIDRALGIPELVNQGQALEPKQD | NCOA3 (aa) |
| 160 | AYTDELVELHRRLMALRERNVLQQIVNLIEETGHENVTNTTEDEDLESLDETTVRKLQ | Tripartite activator- |
| | SCLENGGGGSHEKFPSDLDLDMENGSLECDMESIIRSELMDADGLDENEDSLGGGGSE | ENL FOXO3 |
| | GQSDERALLDQLHTLLSNTDATGLEEIDRALGIPELVNQGQALEPKQD | NCOA3 (aa) |
| 161 | gacgcattggacgattttgatctggatatgctgggaagtgacgccctcgatgattttg | VP64 1 (nt) |
| | accttgacatgcttggttcggatgcccttgatgactttgacctcgacatgctcggcag | |
| | tgacgcccttgatgatttcgacctggacatg | |
| 162 | DALDDFDLDMLGSDALDDFDLDMLGSDALDDFDLDMLGSDALDDEDLDM | VP64 1 (aa) |
| 163 | gacgcgctggacgatttcgatctcgacatgctgggttctgatgccctcgatgactttg | VP64 2 (nt) |
| | acctggatatgttgggaagcgacgcattggatgactttgatctggacatgctcggctc | |
| | cgatgctctggacgatttcgatctcgatatgtta | |
| 164 | DALDDFDLDMLGSDALDDFDLDMLGSDALDDFDLDMLGSDALDDEDLDML | VP64 2 (aa) |
| 165 | GGAATTGTGACCCTGCCTCATCGCTACGTGTACCTGATCAGGTTTCTTGCGGACCACG | Control peptide (nt) |
| | TCGATGCTAGCCAATCCCTAACTACGAGAACTCGTATTGACACATTCATGTGCGTCGC | |
| | CTGCTCTTTCGACGGCAGTAATTATACCGCAGTGTTAAGTCCCCAGATACTCGCCATC | |
| | CGACGAAACTCTTGTGTACGAGATATGCGATTCGTTGCTAGACGTGAGTACACCAAGG | |
| | CCGTGTTC | |
| 166 | GACTACAAAGACCATGACGGTGATTATAAAGATCATGACATCGATTACAAGGATGACG | FLAG epitope tag |
| | ATGACAAG | (nt) |
| 167 | DYKDHDGDYKDHDIDYKDDDDK | FLAG epitope tag |
| | | (aa) |
| 168 | GTTTTAGTACTCTGGAAACAGAATCTACTAAAACAAGGCAAAATGCCGTGTTTATCTC | SaCas9 gRNA |
| | GTCAACTTGTTGGCGAGA | scaffold DNA |
| | | sequence |
| 169 | GUUUUAGUACUCUGGAAACAGAAUCUACUAAAACAAGGCAAAAUGCCGUGUUUAUCUC | SaCas9 gRNA |
| | GUCAACUUGUUGGCGAGA | scaffold RNA |
| | | sequence |
| 170 | GTTTAAGAGCTATGCTGGAAACAGCATAGCAAGTTTAAATAAGGCTAGTCCGTTATCA | SpCas9 gRNA |
| | ACTTGAAAAAGTGGCACCGAGTCGGTGC | scaffold DNA |
| | | sequence |
| 171 | GUUUAAGAGCUAUGCUGGAAACAGCAUAGCAAGUUUAAAUAAGGCUAGUCCGUUAUCA | SpCas9 gRNA |
| | ACUUGAAAAAGUGGCACCGAGUCGGUGC | scaffold RNA |
| | | sequence |
| 172 | AACCCAAAGAAGAAGCGGAAGGTCGGTATCCACGGAGTCCCAGCAGCC | linker (nt) |
| 173 | NPKKKRKVGIHGVPAA | linker (aa) |
| 174 | TCACACAGCTAGGAAGTGGG | FXN promoter- |
| | | targeting gRNA A- |
| | | spacer |
| 175 | TCACACAGCTAGGAAGTGGGGTTTTAGTACTCTGGAAACAGAATCTACTAAAACAAGG | FXN promoter- |
| | CAAAATGCCGTGTTTATCTCGTCAACTTGTTGGCGAGA | targeting gRNA A- |
| | | DNA |
| 176 | LERLKKAKAGEKYKYEVEDFTGVYEEVDEEQYSKLVQARQDDDWIVDDDGIGYVEDGR | DPOLA |
| | EIF | transcriptional |
| | | activation domain |
| | | (aa) |
| 177 | YDNDVTVWSPQGRIHQIEYAME | PSA1 |
| | | transcriptional |
| | | activation domain |
| | | (aa) |
| 178 | LYVGSLHFNITEDMLRGIFEPFGRIESIQLMMDSETGRSKGYGFITFSDSECAKKALE | RBM39 |
| | QLNGFELAGRPM | transcriptional |
| | | activation domain |
| | | (aa) |
| 179 | RKADLENHNKDGGFWTVIDGKVYDIKDFQTQSLTGNSILAQFAGEDPVVALEAALQFE | HERC2 |
| | DTRESMHAFCVGQY | transcriptional |
| | | activation domain |
| | | (aa) |
| 180 | HEKFPSDLDLDMENGSLECDMESIIRSELMDADGLDENEDSLGGGGSEGQSDERALLD | Multipartite |
| | QLHTLLSNTDATGLEEIDRALGIPELVNQGQALEPKQDGGGGSGGGGSGGGGSNPKKK | activator-dSaCas9- |
| | RKVGIHGVPAAKRNYILGLAIGITSVGYGIIDYETRDVIDAGVRLFKEANVENNEGRR | FOXO3 NCOA3 |
| | SKRGARRLKRRRRHRIQRVKKLLFDYNLLTDHSELSGINPYEARVKGLSQKLSEEEFS | (aa) |
| | AALLHLAKRRGVHNVNEVEEDTGNELSTKEQISRNSKALEEKYVAELQLERLKKDGEV | |
| | RGSINRFKTSDYVKEAKQLLKVQKAYHQLDQSFIDTYIDLLETRRTYYEGPGEGSPFG | |
| | WKDIKEWYEMLMGHCTYFPEELRSVKYAYNADLYNALNDLNNLVITRDENEKLEYYEK | |
| | FQIIENVFKQKKKPTLKQIAKEILVNEEDIKGYRVTSTGKPEFTNLKVYHDIKDITAR | |
| | KEIIENAELLDQIAKILTIYQSSEDIQEELTNLNSELTQEEIEQISNLKGYTGTHNLS | |
| | LKAINLILDELWHTNDNQIAIFNRLKLVPKKVDLSQQKEIPTTLVDDFILSPVVKRSF | |
| | IQSIKVINAIIKKYGLPNDIIIELAREKNSKDAQKMINEMQKRNRQTNERIEEIIRTT | |
| | GKENAKYLIEKIKLHDMQEGKCLYSLEAIPLEDLLNNPFNYEVDHIIPRSVSEDNSEN | |
| | NKVLVKQEEASKKGNRTPFQYLSSSDSKISYETFKKHILNLAKGKGRISKTKKEYLLE | |
| | ERDINRFSVQKDFINRNLVDTRYATRGLMNLLRSYFRVNNLDVKVKSINGGFTSELRR | |
| | KWKFKKERNKGYKHHAEDALIIANADFIFKEWKKLDKAKKVMENQMFEEKQAESMPEI | |
| | ETEQEYKEIFITPHQIKHIKDFKDYKYSHRVDKKPNRELINDTLYSTRKDDKGNTLIV | |
| | NNLNGLYDKDNDKLKKLINKSPEKLLMYHHDPQTYQKLKLIMEQYGDEKNPLYKYYEE | |
| | TGNYLTKYSKKDNGPVIKKIKYYGNKLNAHLDITDDYPNSRNKVVKLSLKPYRFDVYL | |
| | DNGVYKFVTVKNLDVIKKENYYEVNSKCYEEAKKLKKISNQAEFIASFYNNDLIKING | |
| | ELYRVIGVNNDLLNRIEVNMIDITYREYLENMNDKRPPRIIKTIASKTQSIKKYSTDI | |
| | LGNLYEVKSKKHPQIIKKG | |
| 181 | PCGICTNEVNDDQDAILCEASCQKWFHRICTGMTETAYGLLTAEASAVWGCDTCMADN | Multipartite |
| | GGGGSHEKFPSDLDLDMENGSLECDMESIIRSELMDADGLDENEDSLGGGGSEGQSDE | activator-dSaCas9- |
| | RALLDQLHTLLSNTDATGLEEIDRALGIPELVNQGQALEPKQDGGGGSGGGGSGGGGS | PYGO1 FOXO3 |
| | NPKKKRKVGIHGVPAAKRNYILGLAIGITSVGYGIIDYETRDVIDAGVRLFKEANVEN | NCOA3 (aa) |
| | NEGRRSKRGARRLKRRRRHRIQRVKKLLFDYNLLTDHSELSGINPYEARVKGLSQKLS | |
| | EEEFSAALLHLAKRRGVHNVNEVEEDTGNELSTKEQISRNSKALEEKYVAELQLERLK | |
| | KDGEVRGSINRFKTSDYVKEAKQLLKVQKAYHQLDQSFIDTYIDLLETRRTYYEGPGE | |
| | GSPFGWKDIKEWYEMLMGHCTYFPEELRSVKYAYNADLYNALNDLNNLVITRDENEKL | |
| | EYYEKFQIIENVFKQKKKPTLKQIAKEILVNEEDIKGYRVTSTGKPEFTNLKVYHDIK | |
| | DITARKEIIENAELLDQIAKILTIYQSSEDIQEELTNLNSELTQEEIEQISNLKGYTG | |
| | THNLSLKAINLILDELWHTNDNQIAIFNRLKLVPKKVDLSQQKEIPTTLVDDFILSPV | |
| | VKRSFIQSIKVINAIIKKYGLPNDIIIELAREKNSKDAQKMINEMQKRNRQTNERIEE | |
| | IIRTTGKENAKYLIEKIKLHDMQEGKCLYSLEAIPLEDLLNNPFNYEVDHIIPRSVSF | |
| | DNSFNNKVLVKQEEASKKGNRTPFQYLSSSDSKISYETFKKHILNLAKGKGRISKTKK | |
| | EYLLEERDINRFSVQKDFINRNLVDTRYATRGLMNLLRSYFRVNNLDVKVKSINGGFT | |
| | SFLRRKWKFKKERNKGYKHHAEDALIIANADFIFKEWKKLDKAKKVMENQMFEEKQAE | |
| | SMPEIETEQEYKEIFITPHQIKHIKDFKDYKYSHRVDKKPNRELINDTLYSTRKDDKG | |
| | NTLIVNNLNGLYDKDNDKLKKLINKSPEKLLMYHHDPQTYQKLKLIMEQYGDEKNPLY | |
| | KYYEETGNYLTKYSKKDNGPVIKKIKYYGNKLNAHLDITDDYPNSRNKVVKLSLKPYR | |
| | FDVYLDNGVYKFVTVKNLDVIKKENYYEVNSKCYEEAKKLKKISNQAEFIASFYNNDL | |
| | IKINGELYRVIGVNNDLLNRIEVNMIDITYREYLENMNDKRPPRIIKTIASKTQSIKK | |
| | YSTDILGNLYEVKSKKHPQIIKKG | |
| 182 | DIDECASNPCRKGATCINGVNGFRCICPEGPHHPSCYNGGGGSHEKFPSDLDLDMENG | Multipartite |
| | SLECDMESIIRSELMDADGLDFNFDSLGGGGSEGQSDERALLDQLHTLLSNTDATGLE | activator-dSaCas9- |
| | EIDRALGIPELVNQGQALEPKQDGGGGSGGGGSGGGGSNPKKKRKVGIHGVPAAKRNY | NOTCH2 FOXO3 |
| | ILGLAIGITSVGYGIIDYETRDVIDAGVRLFKEANVENNEGRRSKRGARRLKRRRRHR | NCOA3 (aa) |
| | IQRVKKLLFDYNLLTDHSELSGINPYEARVKGLSQKLSEEEFSAALLHLAKRRGVHNV | |
| | NEVEEDTGNELSTKEQISRNSKALEEKYVAELQLERLKKDGEVRGSINRFKTSDYVKE | |
| | AKQLLKVQKAYHQLDQSFIDTYIDLLETRRTYYEGPGEGSPFGWKDIKEWYEMLMGHC | |
| | TYFPEELRSVKYAYNADLYNALNDLNNLVITRDENEKLEYYEKFQIIENVFKQKKKPT | |
| | LKQIAKEILVNEEDIKGYRVTSTGKPEFTNLKVYHDIKDITARKEIIENAELLDQIAK | |
| | ILTIYQSSEDIQEELTNLNSELTQEEIEQISNLKGYTGTHNLSLKAINLILDELWHTN | |
| | DNQIAIFNRLKLVPKKVDLSQQKEIPTTLVDDFILSPVVKRSFIQSIKVINAIIKKYG | |
| | LPNDIIIELAREKNSKDAQKMINEMQKRNRQTNERIEEIIRTTGKENAKYLIEKIKLH | |
| | DMQEGKCLYSLEAIPLEDLLNNPFNYEVDHIIPRSVSFDNSFNNKVLVKQEEASKKGN | |
| | RTPFQYLSSSDSKISYETFKKHILNLAKGKGRISKTKKEYLLEERDINRFSVQKDFIN | |
| | RNLVDTRYATRGLMNLLRSYFRVNNLDVKVKSINGGFTSFLRRKWKFKKERNKGYKHH | |
| | AEDALIIANADFIFKEWKKLDKAKKVMENQMFEEKQAESMPEIETEQEYKEIFITPHQ | |
| | IKHIKDFKDYKYSHRVDKKPNRELINDTLYSTRKDDKGNTLIVNNLNGLYDKDNDKLK | |
| | KLINKSPEKLLMYHHDPQTYQKLKLIMEQYGDEKNPLYKYYEETGNYLTKYSKKDNGP | |
| | VIKKIKYYGNKLNAHLDITDDYPNSRNKVVKLSLKPYRFDVYLDNGVYKFVTVKNLDV | |
| | IKKENYYEVNSKCYEEAKKLKKISNQAEFIASFYNNDLIKINGELYRVIGVNNDLLNR | |
| | IEVNMIDITYREYLENMNDKRPPRIIKTIASKTQSIKKYSTDILGNLYEVKSKKHPQI | |
| | IKKG | |
| 183 | EGQSDERALLDQLHTLLSNTDATGLEEIDRALGIPELVNQGQALEPKQDNGGGGSHEK | Multipartite |
| | FPSDLDLDMENGSLECDMESIIRSELMDADGLDENEDSLGGGGSEGQSDERALLDQLH | activator-dSaCas9- |
| | TLLSNTDATGLEEIDRALGIPELVNQGQALEPKQDGGGGSGGGGSGGGGSNPKKKRKV | NCOA3 FOXO3 |
| | GIHGVPAAKRNYILGLAIGITSVGYGIIDYETRDVIDAGVRLFKEANVENNEGRRSKR | NCOA3 (aa) |
| | GARRLKRRRRHRIQRVKKLLFDYNLLTDHSELSGINPYEARVKGLSQKLSEEEFSAAL | |
| | LHLAKRRGVHNVNEVEEDTGNELSTKEQISRNSKALEEKYVAELQLERLKKDGEVRGS | |
| | INRFKTSDYVKEAKQLLKVQKAYHQLDQSFIDTYIDLLETRRTYYEGPGEGSPFGWKD | |
| | IKEWYEMLMGHCTYFPEELRSVKYAYNADLYNALNDLNNLVITRDENEKLEYYEKFQI | |
| | IENVFKQKKKPTLKQIAKEILVNEEDIKGYRVTSTGKPEFTNLKVYHDIKDITARKEI | |
| | IENAELLDQIAKILTIYQSSEDIQEELTNLNSELTQEEIEQISNLKGYTGTHNLSLKA | |
| | INLILDELWHTNDNQIAIFNRLKLVPKKVDLSQQKEIPTTLVDDFILSPVVKRSFIQS | |
| | IKVINAIIKKYGLPNDIIIELAREKNSKDAQKMINEMQKRNRQTNERIEEIIRTTGKE | |
| | NAKYLIEKIKLHDMQEGKCLYSLEAIPLEDLLNNPFNYEVDHIIPRSVSEDNSENNKV | |
| | LVKQEEASKKGNRTPFQYLSSSDSKISYETFKKHILNLAKGKGRISKTKKEYLLEERD | |
| | INRFSVQKDFINRNLVDTRYATRGLMNLLRSYFRVNNLDVKVKSINGGFTSFLRRKWK | |
| | FKKERNKGYKHHAEDALIIANADFIFKEWKKLDKAKKVMENQMFEEKQAESMPEIETE | |
| | QEYKEIFITPHQIKHIKDFKDYKYSHRVDKKPNRELINDTLYSTRKDDKGNTLIVNNL | |
| | NGLYDKDNDKLKKLINKSPEKLLMYHHDPQTYQKLKLIMEQYGDEKNPLYKYYEETGN | |
| | YLTKYSKKDNGPVIKKIKYYGNKLNAHLDITDDYPNSRNKVVKLSLKPYRFDVYLDNG | |
| | VYKFVTVKNLDVIKKENYYEVNSKCYEEAKKLKKISNQAEFIASFYNNDLIKINGELY | |
| | RVIGVNNDLLNRIEVNMIDITYREYLENMNDKRPPRIIKTIASKTQSIKKYSTDILGN | |
| | LYEVKSKKHPQIIKKG | |
| 184 | WFHGAISREDAENLLESQPLGSFLIRVSHSHVGYTLSYKAQSSCCHFMVKLLDDGTFM | Multipartite |
| | IPGEKVAHTSLDALVTFHNGGGGSHEKFPSDLDLDMENGSLECDMESIIRSELMDADG | activator-dSaCas9- |
| | LDFNFDSLGGGGSEGQSDERALLDQLHTLLSNTDATGLEEIDRALGIPELVNQGQALE | HSH2D FOXO3 |
| | PKQDGGGGSGGGGSGGGGSNPKKKRKVGIHGVPAAKRNYILGLAIGITSVGYGIIDYE | NCOA3 (aa) |
| | TRDVIDAGVRLFKEANVENNEGRRSKRGARRLKRRRRHRIQRVKKLLFDYNLLTDHSE | |
| | LSGINPYEARVKGLSQKLSEEEFSAALLHLAKRRGVHNVNEVEEDTGNELSTKEQISR | |
| | NSKALEEKYVAELQLERLKKDGEVRGSINRFKTSDYVKEAKQLLKVQKAYHQLDQSFI | |
| | DTYIDLLETRRTYYEGPGEGSPFGWKDIKEWYEMLMGHCTYFPEELRSVKYAYNADLY | |
| | NALNDLNNLVITRDENEKLEYYEKFQIIENVFKQKKKPTLKQIAKEILVNEEDIKGYR | |
| | VTSTGKPEFTNLKVYHDIKDITARKEIIENAELLDQIAKILTIYQSSEDIQEELTNLN | |
| | SELTQEEIEQISNLKGYTGTHNLSLKAINLILDELWHTNDNQIAIFNRLKLVPKKVDL | |
| | SQQKEIPTTLVDDFILSPVVKRSFIQSIKVINAIIKKYGLPNDIIIELAREKNSKDAQ | |
| | KMINEMQKRNRQTNERIEEIIRTTGKENAKYLIEKIKLHDMQEGKCLYSLEAIPLEDL | |
| | LNNPFNYEVDHIIPRSVSFDNSFNNKVLVKQEEASKKGNRTPFQYLSSSDSKISYETF | |
| | KKHILNLAKGKGRISKTKKEYLLEERDINRFSVQKDFINRNLVDTRYATRGLMNLLRS | |
| | YFRVNNLDVKVKSINGGFTSFLRRKWKFKKERNKGYKHHAEDALIIANADFIFKEWKK | |
| | LDKAKKVMENQMFEEKQAESMPEIETEQEYKEIFITPHQIKHIKDFKDYKYSHRVDKK | |
| | PNRELINDTLYSTRKDDKGNTLIVNNLNGLYDKDNDKLKKLINKSPEKLLMYHHDPQT | |
| | YQKLKLIMEQYGDEKNPLYKYYEETGNYLTKYSKKDNGPVIKKIKYYGNKLNAHLDIT | |
| | DDYPNSRNKVVKLSLKPYRFDVYLDNGVYKFVTVKNLDVIKKENYYEVNSKCYEEAKK | |
| | LKKISNQAEFIASFYNNDLIKINGELYRVIGVNNDLLNRIEVNMIDITYREYLENMND | |
| | KRPPRIIKTIASKTQSIKKYSTDILGNLYEVKSKKHPQIIKKG | |
| 185 | HEKFPSDLDLDMENGSLECDMESIIRSELMDADGLDFNFDSLNGGGGSHEKFPSDLDL | Multipartite |
| | DMFNGSLECDMESIIRSELMDADGLDFNFDSLGGGGSEGQSDERALLDQLHTLLSNTD | activator-dSaCas9- |
| | ATGLEEIDRALGIPELVNQGQALEPKQDGGGGSGGGGSGGGGSNPKKKRKVGIHGVPA | FOXO3 FOXO3 |
| | AKRNYILGLAIGITSVGYGIIDYETRDVIDAGVRLFKEANVENNEGRRSKRGARRLKR | NCOA3 (aa) |
| | RRRHRIQRVKKLLFDYNLLTDHSELSGINPYEARVKGLSQKLSEEEFSAALLHLAKRR | |
| | GVHNVNEVEEDTGNELSTKEQISRNSKALEEKYVAELQLERLKKDGEVRGSINRFKTS | |
| | DYVKEAKQLLKVQKAYHQLDQSFIDTYIDLLETRRTYYEGPGEGSPFGWKDIKEWYEM | |
| | LMGHCTYFPEELRSVKYAYNADLYNALNDLNNLVITRDENEKLEYYEKFQIIENVEKQ | |
| | KKKPTLKQIAKEILVNEEDIKGYRVTSTGKPEFTNLKVYHDIKDITARKEIIENAELL | |
| | DQIAKILTIYQSSEDIQEELTNLNSELTQEEIEQISNLKGYTGTHNLSLKAINLILDE | |
| | LWHTNDNQIAIFNRLKLVPKKVDLSQQKEIPTTLVDDFILSPVVKRSFIQSIKVINAI | |
| | IKKYGLPNDIIIELAREKNSKDAQKMINEMQKRNRQTNERIEEIIRTTGKENAKYLIE | |
| | KIKLHDMQEGKCLYSLEAIPLEDLLNNPFNYEVDHIIPRSVSFDNSENNKVLVKQEEA | |
| | SKKGNRTPFQYLSSSDSKISYETFKKHILNLAKGKGRISKTKKEYLLEERDINRFSVQ | |
| | KDFINRNLVDTRYATRGLMNLLRSYFRVNNLDVKVKSINGGFTSFLRRKWKFKKERNK | |
| | GYKHHAEDALIIANADFIFKEWKKLDKAKKVMENQMFEEKQAESMPEIETEQEYKEIF | |
| | ITPHQIKHIKDFKDYKYSHRVDKKPNRELINDTLYSTRKDDKGNTLIVNNLNGLYDKD | |
| | NDKLKKLINKSPEKLLMYHHDPQTYQKLKLIMEQYGDEKNPLYKYYEETGNYLTKYSK | |
| | KDNGPVIKKIKYYGNKLNAHLDITDDYPNSRNKVVKLSLKPYRFDVYLDNGVYKFVTV | |
| | KNLDVIKKENYYEVNSKCYEEAKKLKKISNQAEFIASFYNNDLIKINGELYRVIGVNN | |
| | DLLNRIEVNMIDITYREYLENMNDKRPPRIIKTIASKTQSIKKYSTDILGNLYEVKSK | |
| | KHPQIIKKG | |
| 186 | ESPSDEGALLDQLYLALRNFDGLEEIDRALGIPELVSQSQAVDPEQFNGGGGSHEKFP | Multipartite |
| | SDLDLDMENGSLECDMESIIRSELMDADGLDENFDSLGGGGSEGQSDERALLDQLHTL | activator-dSaCas9- |
| | LSNTDATGLEEIDRALGIPELVNQGQALEPKQDGGGGSGGGGSGGGGSNPKKKRKVGI | NCOA2 FOXO3 |
| | HGVPAAKRNYILGLAIGITSVGYGIIDYETRDVIDAGVRLFKEANVENNEGRRSKRGA | NCOA3 (aa) |
| | RRLKRRRRHRIQRVKKLLFDYNLLTDHSELSGINPYEARVKGLSQKLSEEEFSAALLH | |
| | LAKRRGVHNVNEVEEDTGNELSTKEQISRNSKALEEKYVAELQLERLKKDGEVRGSIN | |
| | RFKTSDYVKEAKQLLKVQKAYHQLDQSFIDTYIDLLETRRTYYEGPGEGSPFGWKDIK | |
| | EWYEMLMGHCTYFPEELRSVKYAYNADLYNALNDLNNLVITRDENEKLEYYEKFQIIE | |
| | NVFKQKKKPTLKQIAKEILVNEEDIKGYRVTSTGKPEFTNLKVYHDIKDITARKEIIE | |
| | NAELLDQIAKILTIYQSSEDIQEELTNLNSELTQEEIEQISNLKGYTGTHNLSLKAIN | |
| | LILDELWHTNDNQIAIFNRLKLVPKKVDLSQQKEIPTTLVDDFILSPVVKRSFIQSIK | |
| | VINAIIKKYGLPNDIIIELAREKNSKDAQKMINEMQKRNRQTNERIEEIIRTTGKENA | |
| | KYLIEKIKLHDMQEGKCLYSLEAIPLEDLLNNPFNYEVDHIIPRSVSFDNSENNKVLV | |
| | KQEEASKKGNRTPFQYLSSSDSKISYETFKKHILNLAKGKGRISKTKKEYLLEERDIN | |
| | RFSVQKDFINRNLVDTRYATRGLMNLLRSYFRVNNLDVKVKSINGGFTSFLRRKWKFK | |
| | KERNKGYKHHAEDALIIANADFIFKEWKKLDKAKKVMENQMFEEKQAESMPEIETEQE | |
| | YKEIFITPHQIKHIKDFKDYKYSHRVDKKPNRELINDTLYSTRKDDKGNTLIVNNING | |
| | LYDKDNDKLKKLINKSPEKLLMYHHDPQTYQKLKLIMEQYGDEKNPLYKYYEETGNYL | |
| | TKYSKKDNGPVIKKIKYYGNKLNAHLDITDDYPNSRNKVVKLSLKPYRFDVYLDNGVY | |
| | KFVTVKNLDVIKKENYYEVNSKCYEEAKKLKKISNQAEFIASFYNNDLIKINGELYRV | |
| | IGVNNDLLNRIEVNMIDITYREYLENMNDKRPPRIIKTIASKTQSIKKYSTDILGNLY | |
| | EVKSKKHPQIIKKG | |
| 187 | AYTDELVELHRRLMALRERNVLQQIVNLIEETGHFNVTNTTFDFDLFSLDETTVRKLQ | Multipartite |
| | SCLENGGGGSHEKFPSDLDLDMENGSLECDMESIIRSELMDADGLDENFDSLGGGGSE | activator-dSaCas9- |
| | GQSDERALLDQLHTLLSNTDATGLEEIDRALGIPELVNQGQALEPKQDGGGGSGGGGS | ENL FOXO3 |
| | GGGGSNPKKKRKVGIHGVPAAKRNYILGLAIGITSVGYGIIDYETRDVIDAGVRLEKE | NCOA3 (aa) |
| | ANVENNEGRRSKRGARRLKRRRRHRIQRVKKLLFDYNLLTDHSELSGINPYEARVKGL | |
| | SQKLSEEEFSAALLHLAKRRGVHNVNEVEEDTGNELSTKEQISRNSKALEEKYVAELQ | |
| | LERLKKDGEVRGSINRFKTSDYVKEAKQLLKVQKAYHQLDQSFIDTYIDLLETRRTYY | |
| | EGPGEGSPFGWKDIKEWYEMLMGHCTYFPEELRSVKYAYNADLYNALNDLNNLVITRD | |
| | ENEKLEYYEKFQIIENVFKQKKKPTLKQIAKEILVNEEDIKGYRVTSTGKPEFTNLKV | |
| | YHDIKDITARKEIIENAELLDQIAKILTIYQSSEDIQEELTNLNSELTQEEIEQISNL | |
| | KGYTGTHNLSLKAINLILDELWHTNDNQIAIFNRLKLVPKKVDLSQQKEIPTTLVDDF | |
| | ILSPVVKRSFIQSIKVINAIIKKYGLPNDIIIELAREKNSKDAQKMINEMQKRNRQTN | |
| | ERIEEIIRTTGKENAKYLIEKIKLHDMQEGKCLYSLEAIPLEDLLNNPFNYEVDHIIP | |
| | RSVSFDNSFNNKVLVKQEEASKKGNRTPFQYLSSSDSKISYETFKKHILNLAKGKGRI | |
| | SKTKKEYLLEERDINRFSVQKDFINRNLVDTRYATRGLMNLLRSYFRVNNLDVKVKSI | |
| | NGGFTSFLRRKWKFKKERNKGYKHHAEDALIIANADFIFKEWKKLDKAKKVMENQMFE | |
| | EKQAESMPEIETEQEYKEIFITPHQIKHIKDFKDYKYSHRVDKKPNRELINDTLYSTR | |
| | KDDKGNTLIVNNLNGLYDKDNDKLKKLINKSPEKLLMYHHDPQTYQKLKLIMEQYGDE | |
| | KNPLYKYYEETGNYLTKYSKKDNGPVIKKIKYYGNKLNAHLDITDDYPNSRNKVVKLS | |
| | LKPYRFDVYLDNGVYKFVTVKNLDVIKKENYYEVNSKCYEEAKKLKKISNQAEFIASF | |
| | YNNDLIKINGELYRVIGVNNDLLNRIEVNMIDITYREYLENMNDKRPPRIIKTIASKT | |
| | QSIKKYSTDILGNLYEVKSKKHPQIIKKG | |
| 188 | GAGGGCCAGAGCGACGAGAGAGCCCTGCTGGACCAGCTGCACACCCTGCTGAGCAACA | NCOA3- |
| | CCGACGCCACCGGCCTGGAGGAGATCGACAGAGCCCTGGGCATCCCCGAGCTGGTGAA | NCOA3(codon |
| | CCAGGGCCAGGCCCTGGAGCCCAAGCAGGACGGTGGAGGAGGCAGTGAAGGGCAAAGC | optimized)-NCOA3 |
| | GACGAGCGAGCACTCTTGGATCAGCTCCACACGCTGCTCTCAAACACTGATGCCACTG | (nt) |
| | GCTTGGAAGAGATCGATCGAGCATTGGGAATCCCAGAGCTTGTAAATCAAGGTCAGGC | |
| | GCTGGAACCTAAACAGGATGGAGGTGGAGGCTCAGAGGGCCAGAGCGACGAGAGAGCC | |
| | CTGCTGGACCAGCTGCACACCCTGCTGAGCAACACCGACGCCACCGGCCTGGAGGAGA | |
| | TCGACAGAGCCCTGGGCATCCCCGAGCTGGTGAACCAGGGCCAGGCCCTGGAGCCCAA | |
| | GCAGGACGGAGGAGGAGGTTCTGGGGGTGGAGGTAGCGGTGGTGGAGGTAGT | |
| 189 | EGQSDERALLDQLHTLLSNTDATGLEEIDRALGIPELVNQGQALEPKQDGGGGSEGQS | NCOA3- |
| | DERALLDQLHTLLSNTDATGLEEIDRALGIPELVNQGQALEPKQDGGGGSEGQSDERA | NCOA3(codon |
| | LLDQLHTLLSNTDATGLEEIDRALGIPELVNQGQALEPKQDGGGGSGGGGSGGGGS | optimized)-NCOA3 |
| | | (aa) |
| 190 | GCCCGAGGGCGGGGCATGGAC | mouse Nrf2 gRNA |
| | | A spacer |
| 191 | GCGAGAGGAGGATCAACAGTG | mouse Nrf2 gRNA |
| | | B spacer |
| 192 | GGCAGTTGGCCTCTTGCAAAGT | mouse Nrf2 gRNA |
| | | C spacer |
| 193 | PTQAGEGTLSEALLQLQFDDEDLGALLGNSTDPAVFTDLASVDNSEFQQLLNQGIPVA | p65AD (aa) |
| | PHTTEPMLMEYPEAITRLVTGAQRPPDPAPAPLGAPGLPNGLLSGDEDFSSIADMDES | |
| | ALLSQISS | |
| 194 | RDSREGMFLPKPEAGSAISDVFEGREVCQPKRIRPFHPPGSPWANRPLPASLAPTPTG | Rta domain (aa) |
| | PVHEPVGSLTPAPVPQPLDPAPAVTPEASHLLEDPDEETSQAVKALREMADTVIPQKE | |
| | EAAICGQMDLSHPPPRGHLDELTTTLESMTEDLNLDSPLTPELNEILDTELNDECLLH | |
| | AMHISTGLSIFDTSLF | |
| 195 | GFSVDTSALLDLFSPSVTVPDMSLPDLDSSLASIQELLSPQEPPRPPEAENSSPDSGK | HSF1 domain (aa) |
| | QLVHYTAQPLFLLDPGSVDTGSNDLPVLFELGEGSYFSEGDGFAEDPTISLLTGSEPP | |
| | KAKDPTVS | |
| 196 | DALDDFDLDMLGSDALDDFDLDMLGSDALDDFDLDMLGSDALDDEDLDMLGSPKKKRK | VPR (aa) |
| | VGSQYLPDTDDRHRIEEKRKRTYETFKSIMKKSPFSGPTDPRPPPRRIAVPSRSSASV | |
| | PKPAPQPYPFTSSLSTINYDEFPTMVFPSGQISQASALAPAPPQVLPQAPAPAPAPAM | |
| | VSALAQAPAPVPVLAPGPPQAVAPPAPKPTQAGEGTLSEALLQLQFDDEDLGALLGNS | |
| | TDPAVFTDLASVDNSEFQQLLNQGIPVAPHTTEPMLMEYPEAITRLVTGAQRPPDPAP | |
| | APLGAPGLPNGLLSGDEDFSSIADMDFSALLSQISSGSGSGSRDSREGMFLPKPEAGS | |
| | AISDVFEGREVCQPKRIRPFHPPGSPWANRPLPASLAPTPTGPVHEPVGSLTPAPVPQ | |
| | PLDPAPAVTPEASHLLEDPDEETSQAVKALREMADTVIPQKEEAAICGQMDLSHPPPR | |
| | GHLDELTTTLESMTEDLNLDSPLTPELNEILDTFLNDECLLHAMHISTGLSIFDTSLF | |
| 197 | DALDDFDLDMLGSDALDDFDLDMLGSDALDDFDLDMLGSDALDDEDLDMLGSLPSASV | VPH (aa) |
| | EFEGSGGPSGQISNQALALAPSSAPVLAQTMVPSSAMVPLAQPPAPAPVLTPGPPQSL | |
| | SAPVPKSTQAGEGTLSEALLHLQFDADEDLGALLGNSTDPGVFTDLASVDNSEFQQLL | |
| | NQGVSMSHSTAEPMLMEYPEAITRLVTGSQRPPDPAPTPLGTSGLPNGLSGDEDESSI | |
| | ADMDFSALLSQISSSGQGGGGSGFSVDTSALLDLFSPSVTVPDMSLPDLDSSLASIQE | |
| | LLSPQEPPRPPEAENSSPDSGKQLVHYTAQPLFLLDPGSVDTGSNDLPVLFELGEGSY | |
| | FSEGDGFAEDPTISLLTGSEPPKAKDPTVS | |
| 198 | LPTCSCLDRVIQKDKGPYYTHLGAGPSVAAVREIMENRYGQKGNAIRIEIVVYTGKEG | TET1 catalytic |
| | KSSHGCPIAKWVLRRSSDEEKVLCLVRQRTGHHCPTAVMVVLIMVWDGIPLPMADRLY | domain (aa) |
| | TELTENLKSYNGHPTDRRCTLNENRTCTCQGIDPETCGASFSFGCSWSMYFNGCKFGR | |
| | SPSPRRFRIDPSSPLHEKNLEDNLQSLATRLAPIYKQYAPVAYQNQVEYENVARECRL | |
| | GSKEGRPFSGVTACLDFCAHPHRDIHNMNNGSTVVCTLTREDNRSLGVIPQDEQLHVL | |
| | PLYKLSDTDEFGSKEGMEAKIKSGAIEVLAPRRKKRTCFTQPVPRSGKKRAAMMTEVL | |
| | AHKIRAVEKKPIPRIKRKNNSTTTNNSKPSSLPTLGSNTETVQPEVKSETEPHFILKS | |
| | SDNTKTYSLMPSAPHPVKEASPGFSWSPKTASATPAPLKNDATASCGFSERSSTPHCT | |
| | MPSGRLSGANAAAADGPGISQLGEVAPLPTLSAPVMEPLINSEPSTGVTEPLTPHQPN | |
| | HQPSFLTSPQDLASSPMEEDEQHSEADEPPSDEPLSDDPLSPAEEKLPHIDEYWSDSE | |
| | HIFLDANIGGVAIAPAHGSVLIECARRELHATTPVEHPNRNHPTRLSLVFYQHKNLNK | |
| | PQHGFELNKIKFEAKEAKNKKMKASEQKDQAANEGPEQSSEVNELNQIPSHKALTLTH | |
| | DNVVTVSPYALTHVAGPYNHWV | |
| 199 | MAENLLDGPPNPKRAKLSSPGFSANDSTDFGSLFDLENDLPDELIPNGGELGLLNSGN | CREB binding |
| | LVPDAASKHKQLSELLRGGSGSSINPGIGNVSASSPVQQGLGGQAQGQPNSANMASLS | protein (CBP) (AA) |
| | AMGKSPLSQGDSSAPSLPKQAASTSGPTPAASQALNPQAQKQVGLATSSPATSQTGPG | |
| | ICMNANFNQTHPGLLNSNSGHSLINQASQGQAQVMNGSLGAAGRGRGAGMPYPTPAMQ | |
| | GASSSVLAETLTQVSPQMTGHAGLNTAQAGGMAKMGITGNTSPFGQPFSQAGGQPMGA | |
| | TGVNPQLASKQSMVNSLPTFPTDIKNTSVTNVPNMSQMQTSVGIVPTQAIATGPTADP | |
| | EKRKLIQQQLVLLLHAHKCQRREQANGEVRACSLPHCRTMKNVLNHMTHCQAGKACQA | |
| | ILGSPASGIQNTIGSVGTGQQNATSLSNPNPIDPSSMQRAYAALGLPYMNQPQTQLQP | |
| | QVPGQQPAQPQTHQQMRTLNPLGNNPMNIPAGGITTDQQPPNLISESALPTSLGATNP | |
| | LMNDGSNSGNIGTLSTIPTAAPPSSTGVRKGWHEHVTQDLRSHLVHKLVQAIFPTPDP | |
| | AALKDRRMENLVAYAKKVEGDMYESANSRDEYYHLLAEKIYKIQKELEEKRRSRLHKQ | |
| | GILGNQPALPAPGAQPPVIPQAQPVRPPNGPLSLPVNRMQVSQGMNSFNPMSLGNVQL | |
| | PQAPMGPRAASPMNHSVQMNSMGSVPGMAISPSRMPQPPNMMGAHTNNMMAQAPAQSQ | |
| | FLPQNQFPSSSGAMSVGMGQPPAQTGVSQGQVPGAALPNPLNMLGPQASQLPCPPVTQ | |
| | SPLHPTPPPASTAAGMPSLQHTTPPGMTPPQPAAPTQPSTPVSSSGQTPTPTPGSVPS | |
| | ATQTQSTPTVQAAAQAQVTPQPQTPVQPPSVATPQSSQQQPTPVHAQPPGTPLSQAAA | |
| | SIDNRVPTPSSVASAETNSQQPGPDVPVLEMKTETQAEDTEPDPGESKGEPRSEMMEE | |
| | DLQGASQVKEETDIAEQKSEPMEVDEKKPEVKVEVKEEEESSSNGTASQSTSPSQPRK | |
| | KIFKPEELRQALMPTLEALYRQDPESLPFRQPVDPQLLGIPDYFDIVKNPMDLSTIKR | |
| | KLDTGQYQEPWQYVDDVWLMENNAWLYNRKTSRVYKFCSKLAEVFEQEIDPVMQSLGY | |
| | CCGRKYEFSPQTLCCYGKQLCTIPRDAAYYSYQNRYHFCEKCFTEIQGENVTLGDDPS | |
| | QPQTTISKDQFEKKKNDTLDPEPFVDCKECGRKMHQICVLHYDIIWPSGFVCDNCLKK | |
| | TGRPRKENKFSAKRLQTTRLGNHLEDRVNKFLRRQNHPEAGEVFVRVVASSDKTVEVK | |
| | PGMKSRFVDSGEMSESFPYRTKALFAFEEIDGVDVCFFGMHVQEYGSDCPPPNTRRVY | |
| | ISYLDSIHFFRPRCLRTAVYHEILIGYLEYVKKLGYVTGHIWACPPSEGDDYIFHCHP | |
| | PDQKIPKPKRLQEWYKKMLDKAFAERIIHDYKDIFKQATEDRLTSAKELPYFEGDFWP | |
| | NVLEESIKELEQEEEERKKEESTAASETTEGSQGDSKNAKKKNNKKTNKNKSSISRAN | |
| | KKKPSMPNVSNDLSQKLYATMEKHKEVFFVIHLHAGPVINTLPPIVDPDPLLSCDLMD | |
| | GRDAFLTLARDKHWEFSSLRRSKWSTLCMLVELHTQGQDRFVYTCNECKHHVETRWHC | |
| | TVCEDYDLCINCYNTKSHAHKMVKWGLGLDDEGSSQGEPQSKSPQESRRLSIQRCIQS | |
| | LVHACQCRNANCSLPSCQKMKRVVQHTKGCKRKTNGGCPVCKQLIALCCYHAKHCQEN | |
| | KCPVPFCLNIKHKLRQQQIQHRLQQAQLMRRRMATMNTRNVPQQSLPSPTSAPPGTPT | |
| | QQPSTPQTPQPPAQPQPSPVSMSPAGFPSVARTQPPTTVSTGKPTSQVPAPPPPAQPP | |
| | PAAVEAARQIEREAQQQQHLYRVNINNSMPPGRTGMGTPGSQMAPVSLNVPRPNQVSG | |
| | PVMPSMPPGQWQQAPLPQQQPMPGLPRPVISMQAQAAVAGPRMPSVQPPRSISPSALQ | |
| | DLLRTLKSPSSPQQQQQVLNILKSNPQLMAAFIKQRTAKYVANQPGMQPQPGLQSQPG | |
| | MQPQPGMHQQPSLQNLNAMQAGVPRPGVPPQQQAMGGLNPQGQALNIMNPGHNPNMAS | |
| | MNPQYREMLRRQLLQQQQQQQQQQQQQQQQQQGSAGMAGGMAGHGQFQQPQGPGGYPP | |
| | AMQQQQRMQQHLPLQGSSMGQMAAQMGQLGQMGQPGLGADSTPNIQQALQQRILQQQQ | |
| | MKQQIGSPGQPNPMSPQQHMLSGQPQASHLPGQQIATSLSNQVRSPAPVQSPRPQSQP | |
| | PHSSPSPRIQPQPSPHHVSPQTGSPHPGLAVTMASSIDQGHLGNPEQSAMLPQLNTPS | |
| | RSALSSELSLVGDTTGDTLEKFVEGL | |
| 200 | MAENVVEPGPPSAKRPKLSSPALSASASDGTDFGSLEDLEHDLPDELINSTELGLING | p300 (AA) |
| | GDINQLQTSLGMVQDAASKHKQLSELLRSGSSPNLNMGVGGPGQVMASQAQQSSPGLG | |
| | LINSMVKSPMTQAGLTSPNMGMGTSGPNQGPTQSTGMMNSPVNQPAMGMNTGMNAGMN | |
| | PGMLAAGNGQGIMPNQVMNGSIGAGRGRQNMQYPNPGMGSAGNLLTEPLQQGSPQMGG | |
| | QTGLRGPQPLKMGMMNNPNPYGSPYTQNPGQQIGASGLGLQIQTKTVLSNNLSPFAMD | |
| | KKAVPGGGMPNMGQQPAPQVQQPGLVTPVAQGMGSGAHTADPEKRKLIQQQLVLLLHA | |
| | HKCQRREQANGEVRQCNLPHCRTMKNVLNHMTHCQSGKSCQVAHCASSRQIISHWKNC | |
| | TRHDCPVCLPLKNAGDKRNQQPILTGAPVGLGNPSSLGVGQQSAPNLSTVSQIDPSSI | |
| | ERAYAALGLPYQVNQMPTQPQVQAKNQQNQQPGQSPQGMRPMSNMSASPMGVNGGVGV | |
| | QTPSLLSDSMLHSAINSQNPMMSENASVPSLGPMPTAAQPSTTGIRKQWHEDITQDLR | |
| | NHLVHKLVQAIFPTPDPAALKDRRMENLVAYARKVEGDMYESANNRAEYYHLLAEKIY | |
| | KIQKELEEKRRTRLQKQNMLPNAAGMVPVSMNPGPNMGQPQPGMTSNGPLPDPSMIRG | |
| | SVPNQMMPRITPQSGLNQFGQMSMAQPPIVPRQTPPLQHHGQLAQPGALNPPMGYGPR | |
| | MQQPSNQGQFLPQTQFPSQGMNVTNIPLAPSSGQAPVSQAQMSSSSCPVNSPIMPPGS | |
| | QGSHIHCPQLPQPALHQNSPSPVPSRTPTPHHTPPSIGAQQPPATTIPAPVPTPPAMP | |
| | PGPQSQALHPPPRQTPTPPTTQLPQQVQPSLPAAPSADQPQQQPRSQQSTAASVPTPT | |
| | APLLPPQPATPLSQPAVSIEGQVSNPPSTSSTEVNSQAIAEKQPSQEVKMEAKMEVDQ | |
| | PEPADTQPEDISESKVEDCKMESTETEERSTELKTEIKEEEDQPSTSATQSSPAPGQS | |
| | KKKIFKPEELRQALMPTLEALYRQDPESLPFRQPVDPQLLGIPDYFDIVKSPMDLSTI | |
| | KRKLDTGQYQEPWQYVDDIWLMFNNAWLYNRKTSRVYKYCSKLSEVFEQEIDPVMQSL | |
| | GYCCGRKLEFSPQTLCCYGKQLCTIPRDATYYSYQNRYHFCEKCFNEIQGESVSLGDD | |
| | PSQPQTTINKEQFSKRKNDTLDPELFVECTECGRKMHQICVLHHEIIWPAGFVCDGCL | |
| | KKSARTRKENKFSAKRLPSTRLGTFLENRVNDFLRRQNHPESGEVTVRVVHASDKTVE | |
| | VKPGMKARFVDSGEMAESFPYRTKALFAFEEIDGVDLCFFGMHVQEYGSDCPPPNQRR | |
| | VYISYLDSVHFFRPKCLRTAVYHEILIGYLEYVKKLGYTTGHIWACPPSEGDDYIFHC | |
| | HPPDQKIPKPKRLQEWYKKMLDKAVSERIVHDYKDIFKQATEDRLTSAKELPYFEGDF | |
| | WPNVLEESIKELEQEEEERKREENTSNESTDVTKGDSKNAKKKNNKKTSKNKSSLSRG | |
| | NKKKPGMPNVSNDLSQKLYATMEKHKEVFFVIRLIAGPAANSLPPIVDPDPLIPCDLM | |
| | DGRDAFLTLARDKHLEFSSLRRAQWSTMCMLVELHTQSQDRFVYTCNECKHHVETRWH | |
| | CTVCEDYDLCITCYNTKNHDHKMEKLGLGLDDESNNQQAAATQSPGDSRRLSIQRCIQ | |
| | SLVHACQCRNANCSLPSCQKMKRVVQHTKGCKRKTNGGCPICKQLIALCCYHAKHCQE | |
| | NKCPVPFCLNIKQKLRQQQLQHRLQQAQMLRRRMASMQRTGVVGQQQGLPSPTPATPT | |
| | TPTGQQPTTPQTPQPTSQPQPTPPNSMPPYLPRTQAAGPVSQGKAAGQVTPPTPPQTA | |
| | QPPLPGPPPAAVEMAMQIQRAAETQRQMAHVQIFQRPIQHQMPPMTPMAPMGMNPPPM | |
| | TRGPSGHLEPGMGPTGMQQQPPWSQGGLPQPQQLQSGMPRPAMMSVAQHGQPLNMAPQ | |
| | PGLGQVGISPLKPGTVSQQALQNLLRTLRSPSSPLQQQQVLSILHANPQLLAAFIKQR | |
| | AAKYANSNPQPIPGQPGMPQGQPGLQPPTMPGQQGVHSNPAMQNMNPMQAGVQRAGLP | |
| | QQQPQQQLQPPMGGMSPQAQQMNMNHNTMPSQFRDILRRQQMMQQQQQQGAGPGIGPG | |
| | MANHNQFQQPQGVGYPPQQQQRMQHHMQQMQQGNMGQIGQLPQALGAEAGASLQAYQQ | |
| | RLLQQQMGSPVQPNPMSPQQHMLPNQAQSPHLQGQQIPNSLSNQVRSPQPVPSPRPQS | |
| | QPPHSSPSPRMQPQPSPHHVSPQTSSPHPGLVAAQANPMEQGHFASPDQNSMLSQLAS | |
| | NPGMANLHGASATDLGLSTDNSDLNSNLSQSTLDIH | |
| 201 | IFKPEELRQALMPTLEALYRQDPESLPFRQPVDPQLLGIPDYFDIVKSPMDLSTIKRK | p300 domain (AA) |
| | LDTGQYQEPWQYVDDIWLMFNNAWLYNRKTSRVYKYCSKLSEVFEQEIDPVMQSLGYC | |
| | CGRKLEFSPQTLCCYGKQLCTIPRDATYYSYQNRYHFCEKCFNEIQGESVSLGDDPSQ | |
| | PQTTINKEQFSKRKNDTLDPELFVECTECGRKMHQICVLHHEIIWPAGFVCDGCLKKS | |
| | ARTRKENKFSAKRLPSTRLGTFLENRVNDFLRRQNHPESGEVTVRVVHASDKTVEVKP | |
| | GMKARFVDSGEMAESFPYRTKALFAFEEIDGVDLCFFGMHVQEYGSDCPPPNQRRVYI | |
| | SYLDSVHFFRPKCLRTAVYHEILIGYLEYVKKLGYTTGHIWACPPSEGDDYIFHCHPP | |
| | DQKIPKPKRLQEWYKKMLDKAVSERIVHDYKDIFKQATEDRLTSAKELPYFEGDFWPN | |
| | VLEESIKELEQEEEERKREENTSNESTDVTKGDSKNAKKKNNKKTSKNKSSLSRGNKK | |
| | KPGMPNVSNDLSQKLYATMEKHKEVFFVIRLIAGPAANSLPPIVDPDPLIPCDLMDGR | |
| | DAFLTLARDKHLEFSSLRRAQWSTMCMLVELHTQSQD | |
| 202 | MDLPVGPGAAGPSNVPAFLTKLWTLVSDPDTDALICWSPSGNSFHVFDQGQFAKEVLP | HSF1 (AA) |
| | KYFKHNNMASFVRQLNMYGFRKVVHIEQGGLVKPERDDTEFQHPCFLRGQEQLLENIK | |
| | RKVTSVSTLKSEDIKIRQDSVTKLLTDVQLMKGKQECMDSKLLAMKHENEALWREVAS | |
| | LRQKHAQQQKVVNKLIQFLISLVQSNRILGVKRKIPLMLNDSGSAHSMPKYSRQFSLE | |
| | HVHGSGPYSAPSPAYSSSSLYAPDAVASSGPIISDITELAPASPMASPGGSIDERPLS | |
| | SSPLVRVKEEPPSPPQSPRVEEASPGRPSSVDTLLSPTALIDSILRESEPAPASVTAL | |
| | TDARGHTDTEGRPPSPPPTSTPEKCLSVACLDKNELSDHLDAMDSNLDNLQTMLSSHG | |
| | FSVDTSALLDLFSPSVTVPDMSLPDLDSSLASIQELLSPQEPPRPPEAENSSPDSAGA | |
| | LHSAAAVPAGPRLRGHREQRPAGAV | |
| 203 | MSRSRHARPSRLVRKEDVNKKKKNSQLRKTTKGANKNVASVKTLSPGKLKQLIQERDV | TET1 (AA) |
| | KKKTEPKPPVPVRSLLTRAGAARMNLDRTEVLFQNPESLTCNGFTMALRSTSLSRRLS | |
| | QPPLVVAKSKKVPLSKGLEKQHDCDYKILPALGVKHSENDSVPMQDTQVLPDIETLIG | |
| | VQNPSLLKGKSQETTQFWSQRVEDSKINIPTHSGPAAEILPGPLEGTRCGEGLESEET | |
| | LNDTSGSPKMFAQDTVCAPFPQRATPKVTSQGNPSIQLEELGSRVESLKLSDSYLDPI | |
| | KSEHDCYPTSSLNKVIPDLNLRNCLALGGSTSPTSVIKFLLAGSKQATLGAKPDHQEA | |
| | FEATANQQEVSDTTSFLGQAFGAIPHQWELPGADPVHGEALGETPDLPEIPGAIPVQG | |
| | EVFGTILDQQETLGMSGSVVPDLPVFLPVPPNPIATFNAPSKWPEPQSTVSYGLAVQG | |
| | AIQILPLGSGHTPQSSSNSEKNSLPPVMAISNVENEKQVHISFLPANTQGFPLAPERG | |
| | LFHASLGIAQLSQAGPSKSDRGSSQVSVTSTVHVVNTTVVTMPVPMVSTSSSSYTTLL | |
| | PTLEKKKRKRCGVCEPCQQKTNCGECTYCKNRKNSHQICKKRKCEELKKKPSVVVPLE | |
| | VIKENKRPQREKKPKVLKADFDNKPVNGPKSESMDYSRCGHGEEQKLELNPHTVENVT | |
| | KNEDSMTGIEVEKWTQNKKSQLTDHVKGDESANVPEAEKSKNSEVDKKRTKSPKLFVQ | |
| | TVRNGIKHVHCLPAETNVSFKKFNIEEFGKTLENNSYKFLKDTANHKNAMSSVATDMS | |
| | CDHLKGRSNVLVFQQPGENCSSIPHSSHSIINHHASIHNEGDQPKTPENIPSKEPKDG | |
| | SPVQPSLLSLMKDRRLTLEQVVAIEALTQLSEAPSENSSPSKSEKDEESEQRTASLLN | |
| | SCKAILYTVRKDLQDPNLQGEPPKLNHCPSLEKQSSCNTVVFNGQTTTLSNSHINSAT | |
| | NQASTKSHEYSKVTNSLSLFIPKSNSSKIDTNKSIAQGIITLDNCSNDLHQLPPRNNE | |
| | VEYCNQLLDSSKKLDSDDLSCQDATHTQIEEDVATQLTQLASIIKINYIKPEDKKVES | |
| | TPTSLVTCNVQQKYNQEKGTIQQKPPSSVHNNHGSSLTKQKNPTQKKTKSTPSRDRRK | |
| | KKPTVVSYQENDRQKWEKLSYMYGTICDIWIASKFQNFGQFCPHDFPTVFGKISSSTK | |
| | IWKPLAQTRSIMQPKTVFPPLTQIKLQRYPESAEEKVKVEPLDSLSLFHLKTESNGKA | |
| | FTDKAYNSQVQLTVNANQKAHPLTQPSSPPNQCANVMAGDDQIRFQQVVKEQLMHQRL | |
| | PTLPGISHETPLPESALTLRNVNVVCSGGITVVSTKSEEEVCSSSFGTSEFSTVDSAQ | |
| | KNFNDYAMNFFTNPTKNLVSITKDSELPTCSCLDRVIQKDKGPYYTHLGAGPSVAAVR | |
| | EIMENRYGQKGNAIRIEIVVYTGKEGKSSHGCPIAKWVLRRSSDEEKVLCLVRQRTGH | |
| | HCPTAVMVVLIMVWDGIPLPMADRLYTELTENLKSYNGHPTDRRCTLNENRTCTCQGI | |
| | DPETCGASFSFGCSWSMYFNGCKFGRSPSPRRFRIDPSSPLHEKNLEDNLQSLATRLA | |
| | PIYKQYAPVAYQNQVEYENVARECRLGSKEGRPFSGVTACLDFCAHPHRDIHNMNNGS | |
| | TVVCTLTREDNRSLGVIPQDEQLHVLPLYKLSDTDEFGSKEGMEAKIKSGAIEVLAPR | |
| | RKKRTCFTQPVPRSGKKRAAMMTEVLAHKIRAVEKKPIPRIKRKNNSTTTNNSKPSSL | |
| | PTLGSNTETVQPEVKSETEPHFILKSSDNTKTYSLMPSAPHPVKEASPGFSWSPKTAS | |
| | ATPAPLKNDATASCGFSERSSTPHCTMPSGRLSGANAAAADGPGISQLGEVAPLPTLS | |
| | APVMEPLINSEPSTGVTEPLTPHQPNHQPSELTSPQDLASSPMEEDEQHSEADEPPSD | |
| | EPLSDDPLSPAEEKLPHIDEYWSDSEHIFLDANIGGVAIAPAHGSVLIECARRELHAT | |
| | TPVEHPNRNHPTRLSLVFYQHKNLNKPQHGFELNKIKFEAKEAKNKKMKASEQKDQAA | |
| | NEGPEQSSEVNELNQIPSHKALTLTHDNVVTVSPYALTHVAGPYNHWV | |
| 204 | LLPKNYHLENEVARLKKLVGERGGSGG | SunTag repeat V1 |
| 205 | EELLSKNYHLENEVARLKK | SunTag GCN4 |
| | | peptide V2 (AA) |
| 206 | GSGSG | linker (SunTag) |
| 207 | EELLSKNYHLENEVARLKKGSGSG | SunTag repeat V4 |
| 208 | TCACACAGCTAGGAAGTGGG | target site A or |
| | | gRNA spacer |
| 209 | GAGGCTGCTTGGCCGCCGGT | target site B or |
| | | gRNA spacer |
| 210 | GCCCGCTCCGCCCTCCAGCG | target site C or |
| | | gRNA spacer |
| 211 | AGCCTGCTTTGTGCAAAGCA | target site D or |
| | | gRNA spacer |
| 212 | CCGGCCGATGACGCGCCGCGGG | target site E or |
| | | gRNA spacer |
| 213 | GGCCGGCTACTGCGCGGCGCCC | target site F or |
| | | gRNA spacer |
| 214 | ACCTCTAGCTGCTCCCCCACAG | target site G or |
| | | gRNA spacer |
| 215 | CCGCCCGCTCCGCCCTCCAGCG | target site H or |
| | | gRNA spacer |
| 216 | GCCCGAGAGTCCACATGCTGCT | target site I or |
| | | gRNA spacer |
| 217 | GCTCTCCATTTTTGTTAAATGC | target site J or |
| | | gRNA spacer |
| 218 | CTGCTGTAAACCCATACCGG | target site K or |
| | | gRNA spacer |
| 219 | GCAGAGTACAGATTTACACA | target site L or |
| | | gRNA spacer |
| 220 | CAAGGGAGACTGCAGCCTGG | target site M or |
| | | gRNA spacer |
| 221 | AAGCTGGGAAGTTCTTCCTG | target site N or |
| | | gRNA spacer |
| 222 | ATGCACGAATAGTGCTAAGC | target site O or |
| | | gRNA spacer |
| 223 | TACACAAGGCATCCGTCTCC | target site P or |
| | | gRNA spacer |
| 224 | GGCTGCTTGGCCGCCGGTAT | target site Q or |
| | | gRNA spacer |
| 225 | TTTAGAAGCGGCGGGCCACC | target site R or |
| | | gRNA spacer |
| 226 | AATTGAGGCTGCTTGGCCGC | target site S or |
| | | gRNA spacer |
| 227 | GCAAAGCACGGAGTGCAACC | target site T or |
| | | gRNA spacer |
| 228 | TGCATGGGACAACCACCACGA | target site U or |
| | | gRNA spacer |
| 229 | UCACACAGCUAGGAAGUGGG | gRNA A spacer |
| | | RNA |
| 230 | GAGGCUGCUUGGCCGCCGGU | gRNA B spacer |
| | | RNA |
| 231 | GCCCGCUCCGCCCUCCAGCG | gRNA C spacer |
| | | RNA |
| 232 | AGCCUGCUUUGUGCAAAGCA | gRNA D spacer |
| | | RNA |
| 233 | CCGGCCGAUGACGCGCCGCGGG | gRNA E spacer |
| | | RNA |
| 234 | GGCCGGCUACUGCGCGGCGCCC | gRNA F spacer |
| | | RNA |
| 235 | ACCUCUAGCUGCUCCCCCACAG | gRNA G spacer |
| | | RNA |
| 236 | CCGCCCGCUCCGCCCUCCAGCG | gRNA H spacer |
| | | RNA |
| 237 | GCCCGAGAGUCCACAUGCUGCU | gRNA I spacer |
| | | RNA |
| 238 | GCUCUCCAUUUUUGUUAAAUGC | gRNA J spacer |
| | | RNA |
| 239 | CUGCUGUAAACCCAUACCGG | gRNA K spacer |
| | | RNA |
| 240 | GCAGAGUACAGAUUUACACA | gRNA L spacer |
| | | RNA |
| 241 | CAAGGGAGACUGCAGCCUGG | gRNA M spacer |
| | | RNA |
| 242 | AAGCUGGGAAGUUCUUCCUG | gRNA N spacer |
| | | RNA |
| 243 | AUGCACGAAUAGUGCUAAGC | gRNA O spacer |
| | | RNA |
| 244 | UACACAAGGCAUCCGUCUCC | gRNA P spacer |
| | | RNA |
| 245 | GGCUGCUUGGCCGCCGGUAU | gRNA Q spacer |
| | | RNA |
| 246 | UUUAGAAGCGGCGGGCCACC | gRNA R spacer |
| | | RNA |
| 247 | AAUUGAGGCUGCUUGGCCGC | gRNA S spacer |
| | | RNA |
| 248 | GCAAAGCACGGAGUGCAACC | gRNA T spacer |
| | | RNA |
| 249 | UGCAUGGGACAACCACCACGA | gRNA U spacer |
| | | RNA |
| 250 | UCACACAGCUAGGAAGUGGGGUUUUAGUACUCUGGAAACAGAAUCUACUAAAACAAGG | gRNA A |
| | CAAAAUGCCGUGUUUAUCUCGUCAACUUGUUGGCGAGA | |
| 251 | GAGGCUGCUUGGCCGCCGGUGUUUUAGUACUCUGGAAACAGAAUCUACUAAAACAAGG | gRNA B |
| | CAAAAUGCCGUGUUUAUCUCGUCAACUUGUUGGCGAGA | |
| 252 | GCCCGCUCCGCCCUCCAGCGGUUUUAGUACUCUGGAAACAGAAUCUACUAAAACAAGG | gRNA C |
| | CAAAAUGCCGUGUUUAUCUCGUCAACUUGUUGGCGAGA | |
| 253 | AGCCUGCUUUGUGCAAAGCAGUUUUAGUACUCUGGAAACAGAAUCUACUAAAACAAGG | gRNA D |
| | CAAAAUGCCGUGUUUAUCUCGUCAACUUGUUGGCGAGA | |
| 254 | CCGGCCGAUGACGCGCCGCGGGGUUUUAGUACUCUGGAAACAGAAUCUACUAAAACAA | gRNA E |
| | GGCAAAAUGCCGUGUUUAUCUCGUCAACUUGUUGGCGAGA | |
| 255 | GGCCGGCUACUGCGCGGCGCCCGUUUUAGUACUCUGGAAACAGAAUCUACUAAAACAA | gRNA F |
| | GGCAAAAUGCCGUGUUUAUCUCGUCAACUUGUUGGCGAGA | |
| 256 | ACCUCUAGCUGCUCCCCCACAGGUUUUAGUACUCUGGAAACAGAAUCUACUAAAACAA | gRNA G |
| | GGCAAAAUGCCGUGUUUAUCUCGUCAACUUGUUGGCGAGA | |
| 257 | CCGCCCGCUCCGCCCUCCAGCGGUUUUAGUACUCUGGAAACAGAAUCUACUAAAACAA | gRNA H |
| | GGCAAAAUGCCGUGUUUAUCUCGUCAACUUGUUGGCGAGA | |
| 258 | GCCCGAGAGUCCACAUGCUGCUGUUUUAGUACUCUGGAAACAGAAUCUACUAAAACAA | gRNA I |
| | GGCAAAAUGCCGUGUUUAUCUCGUCAACUUGUUGGCGAGA | |
| 259 | GCUCUCCAUUUUUGUUAAAUGCGUUUUAGUACUCUGGAAACAGAAUCUACUAAAACAA | gRNA J |
| | GGCAAAAUGCCGUGUUUAUCUCGUCAACUUGUUGGCGAGA | |
| 260 | CUGCUGUAAACCCAUACCGGGUUUAAGAGCUAUGCUGGAAACAGCAUAGCAAGUUUAA | gRNA K |
| | AUAAGGCUAGUCCGUUAUCAACUUGAAAAAGUGGCACCGAGUCGGUGC | |
| 261 | GCAGAGUACAGAUUUACACAGUUUAAGAGCUAUGCUGGAAACAGCAUAGCAAGUUUAA | gRNA L |
| | AUAAGGCUAGUCCGUUAUCAACUUGAAAAAGUGGCACCGAGUCGGUGC | |
| 262 | CAAGGGAGACUGCAGCCUGGGUUUAAGAGCUAUGCUGGAAACAGCAUAGCAAGUUUAA | gRNA M |
| | AUAAGGCUAGUCCGUUAUCAACUUGAAAAAGUGGCACCGAGUCGGUGC | |
| 263 | AAGCUGGGAAGUUCUUCCUGGUUUAAGAGCUAUGCUGGAAACAGCAUAGCAAGUUUAA | gRNA N |
| | AUAAGGCUAGUCCGUUAUCAACUUGAAAAAGUGGCACCGAGUCGGUGC | |
| 264 | AUGCACGAAUAGUGCUAAGCGUUUAAGAGCUAUGCUGGAAACAGCAUAGCAAGUUUAA | gRNA O |
| | AUAAGGCUAGUCCGUUAUCAACUUGAAAAAGUGGCACCGAGUCGGUGC | |
| 265 | UACACAAGGCAUCCGUCUCCGUUUAAGAGCUAUGCUGGAAACAGCAUAGCAAGUUUAA | gRNA P |
| | AUAAGGCUAGUCCGUUAUCAACUUGAAAAAGUGGCACCGAGUCGGUGC | |
| 266 | GGCUGCUUGGCCGCCGGUAUGUUUAAGAGCUAUGCUGGAAACAGCAUAGCAAGUUUAA | gRNA Q |
| | AUAAGGCUAGUCCGUUAUCAACUUGAAAAAGUGGCACCGAGUCGGUGC | |
| 267 | UUUAGAAGCGGCGGGCCACCGUUUAAGAGCUAUGCUGGAAACAGCAUAGCAAGUUUAA | gRNA R |
| | AUAAGGCUAGUCCGUUAUCAACUUGAAAAAGUGGCACCGAGUCGGUGC | |
| 268 | AAUUGAGGCUGCUUGGCCGCGUUUAAGAGCUAUGCUGGAAACAGCAUAGCAAGUUUAA | gRNA S |
| | AUAAGGCUAGUCCGUUAUCAACUUGAAAAAGUGGCACCGAGUCGGUGC | |
| 269 | GCAAAGCACGGAGUGCAACCGUUUAAGAGCUAUGCUGGAAACAGCAUAGCAAGUUUAA | gRNA T |
| | AUAAGGCUAGUCCGUUAUCAACUUGAAAAAGUGGCACCGAGUCGGUGC | |
| 270 | UGCAUGGGACAACCACCACGAGUUUUAGUACUCUGGAAACAGAAUCUACUAAAACAAG | gRNA U |
| | GCAAAAUGCCGUGUUUAUCUCGUCAACUUGUUGGCGAGA | |
| 271 | MDYKDHDGDYKDHDIDYKDDDDKHGPRHEKFPSDLDLDMENGSLECDMESIIRSELMD | Multipartite |
| | ADGLDFNFDSLGGGGSEGQSDERALLDQLHTLLSNTDATGLEEIDRALGIPELVNQGQ | activator-dSaCas9- |
| | ALEPKQDGGGGSGGGGSGGGGSNPKKKRKVGIHGVPAAKRNYILGLAIGITSVGYGII | FOXO3 NCOA3 |
| | DYETRDVIDAGVRLFKEANVENNEGRRSKRGARRLKRRRRHRIQRVKKLLFDYNLLTD | (aa) |
| | HSELSGINPYEARVKGLSQKLSEEEFSAALLHLAKRRGVHNVNEVEEDTGNELSTKEQ | |
| | ISRNSKALEEKYVAELQLERLKKDGEVRGSINRFKTSDYVKEAKQLLKVQKAYHQLDQ | |
| | SFIDTYIDLLETRRTYYEGPGEGSPFGWKDIKEWYEMLMGHCTYFPEELRSVKYAYNA | |
| | DLYNALNDLNNLVITRDENEKLEYYEKFQIIENVFKQKKKPTLKQIAKEILVNEEDIK | |
| | GYRVTSTGKPEFTNLKVYHDIKDITARKEIIENAELLDQIAKILTIYQSSEDIQEELT | |
| | NLNSELTQEEIEQISNLKGYTGTHNLSLKAINLILDELWHTNDNQIAIFNRLKLVPKK | |
| | VDLSQQKEIPTTLVDDFILSPVVKRSFIQSIKVINAIIKKYGLPNDIIIELAREKNSK | |
| | DAQKMINEMQKRNRQTNERIEEIIRTTGKENAKYLIEKIKLHDMQEGKCLYSLEAIPL | |
| | EDLLNNPFNYEVDHIIPRSVSFDNSFNNKVLVKQEEASKKGNRTPFQYLSSSDSKISY | |
| | ETFKKHILNLAKGKGRISKTKKEYLLEERDINRFSVQKDFINRNLVDTRYATRGLMNL | |
| | LRSYFRVNNLDVKVKSINGGFTSFLRRKWKFKKERNKGYKHHAEDALIIANADFIFKE | |
| | WKKLDKAKKVMENQMFEEKQAESMPEIETEQEYKEIFITPHQIKHIKDFKDYKYSHRV | |
| | DKKPNRELINDTLYSTRKDDKGNTLIVNNLNGLYDKDNDKLKKLINKSPEKLLMYHHD | |
| | PQTYQKLKLIMEQYGDEKNPLYKYYEETGNYLTKYSKKDNGPVIKKIKYYGNKLNAHL | |
| | DITDDYPNSRNKVVKLSLKPYRFDVYLDNGVYKFVTVKNLDVIKKENYYEVNSKCYEE | |
| | AKKLKKISNQAEFIASFYNNDLIKINGELYRVIGVNNDLLNRIEVNMIDITYREYLEN | |
| | MNDKRPPRIIKTIASKTQSIKKYSTDILGNLYEVKSKKHPQIIKKGKRPAATKKAGQA | |
| | KKKKAS | |
| 272 | MDYKDHDGDYKDHDIDYKDDDDKHGPRPCGICTNEVNDDQDAILCEASCQKWFHRICT | Multipartite |
| | GMTETAYGLLTAEASAVWGCDTCMADNGGGGSHEKFPSDLDLDMENGSLECDMESIIR | activator-dSaCas9- |
| | SELMDADGLDFNFDSLGGGGSEGQSDERALLDQLHTLLSNTDATGLEEIDRALGIPEL | PYGO1 FOXO3 |
| | VNQGQALEPKQDGGGGSGGGGSGGGGSNPKKKRKVGIHGVPAAKRNYILGLAIGITSV | NCOA3 (aa) |
| | GYGIIDYETRDVIDAGVRLFKEANVENNEGRRSKRGARRLKRRRRHRIQRVKKLLFDY | |
| | NLLTDHSELSGINPYEARVKGLSQKLSEEEFSAALLHLAKRRGVHNVNEVEEDTGNEL | |
| | STKEQISRNSKALEEKYVAELQLERLKKDGEVRGSINRFKTSDYVKEAKQLLKVQKAY | |
| | HQLDQSFIDTYIDLLETRRTYYEGPGEGSPFGWKDIKEWYEMLMGHCTYFPEELRSVK | |
| | YAYNADLYNALNDLNNLVITRDENEKLEYYEKFQIIENVFKQKKKPTLKQIAKEILVN | |
| | EEDIKGYRVTSTGKPEFTNLKVYHDIKDITARKEIIENAELLDQIAKILTIYQSSEDI | |
| | QEELTNLNSELTQEEIEQISNLKGYTGTHNLSLKAINLILDELWHTNDNQIAIFNRLK | |
| | LVPKKVDLSQQKEIPTTLVDDFILSPVVKRSFIQSIKVINAIIKKYGLPNDIIIELAR | |
| | EKNSKDAQKMINEMQKRNRQTNERIEEIIRTTGKENAKYLIEKIKLHDMQEGKCLYSL | |
| | EAIPLEDLLNNPFNYEVDHIIPRSVSFDNSFNNKVLVKQEEASKKGNRTPFQYLSSSD | |
| | SKISYETFKKHILNLAKGKGRISKTKKEYLLEERDINRFSVQKDFINRNLVDTRYATR | |
| | GLMNLLRSYFRVNNLDVKVKSINGGFTSFLRRKWKFKKERNKGYKHHAEDALIIANAD | |
| | FIFKEWKKLDKAKKVMENQMFEEKQAESMPEIETEQEYKEIFITPHQIKHIKDEKDYK | |
| | YSHRVDKKPNRELINDTLYSTRKDDKGNTLIVNNLNGLYDKDNDKLKKLINKSPEKLL | |
| | MYHHDPQTYQKLKLIMEQYGDEKNPLYKYYEETGNYLTKYSKKDNGPVIKKIKYYGNK | |
| | LNAHLDITDDYPNSRNKVVKLSLKPYRFDVYLDNGVYKFVTVKNLDVIKKENYYEVNS | |
| | KCYEEAKKLKKISNQAEFIASFYNNDLIKINGELYRVIGVNNDLLNRIEVNMIDITYR | |
| | EYLENMNDKRPPRIIKTIASKTQSIKKYSTDILGNLYEVKSKKHPQIIKKGKRPAATK | |
| | KAGQAKKKKAS | |
| 273 | MDYKDHDGDYKDHDIDYKDDDDKHGPRDIDECASNPCRKGATCINGVNGFRCICPEGP | Multipartite |
| | HHPSCYNGGGGSHEKFPSDLDLDMFNGSLECDMESIIRSELMDADGLDFNFDSLGGGG | activator-dSaCas9- |
| | SEGQSDERALLDQLHTLLSNTDATGLEEIDRALGIPELVNQGQALEPKQDGGGGSGGG | NOTCH2 FOXO3 |
| | GSGGGGSNPKKKRKVGIHGVPAAKRNYILGLAIGITSVGYGIIDYETRDVIDAGVRLE | NCOA3 (aa) |
| | KEANVENNEGRRSKRGARRLKRRRRHRIQRVKKLLFDYNLLTDHSELSGINPYEARVK | |
| | GLSQKLSEEEFSAALLHLAKRRGVHNVNEVEEDTGNELSTKEQISRNSKALEEKYVAE | |
| | LQLERLKKDGEVRGSINRFKTSDYVKEAKQLLKVQKAYHQLDQSFIDTYIDLLETRRT | |
| | YYEGPGEGSPFGWKDIKEWYEMLMGHCTYFPEELRSVKYAYNADLYNALNDLNNLVIT | |
| | RDENEKLEYYEKFQIIENVFKQKKKPTLKQIAKEILVNEEDIKGYRVTSTGKPEFTNL | |
| | KVYHDIKDITARKEIIENAELLDQIAKILTIYQSSEDIQEELTNLNSELTQEEIEQIS | |
| | NLKGYTGTHNLSLKAINLILDELWHTNDNQIAIFNRLKLVPKKVDLSQQKEIPTTLVD | |
| | DFILSPVVKRSFIQSIKVINAIIKKYGLPNDIIIELAREKNSKDAQKMINEMQKRNRQ | |
| | TNERIEEIIRTTGKENAKYLIEKIKLHDMQEGKCLYSLEAIPLEDLLNNPFNYEVDHI | |
| | IPRSVSFDNSFNNKVLVKQEEASKKGNRTPFQYLSSSDSKISYETFKKHILNLAKGKG | |
| | RISKTKKEYLLEERDINRFSVQKDFINRNLVDTRYATRGLMNLLRSYFRVNNLDVKVK | |
| | SINGGFTSFLRRKWKFKKERNKGYKHHAEDALIIANADFIFKEWKKLDKAKKVMENQM | |
| | FEEKQAESMPEIETEQEYKEIFITPHQIKHIKDFKDYKYSHRVDKKPNRELINDTLYS | |
| | TRKDDKGNTLIVNNLNGLYDKDNDKLKKLINKSPEKLLMYHHDPQTYQKLKLIMEQYG | |
| | DEKNPLYKYYEETGNYLTKYSKKDNGPVIKKIKYYGNKLNAHLDITDDYPNSRNKVVK | |
| | LSLKPYRFDVYLDNGVYKFVTVKNLDVIKKENYYEVNSKCYEEAKKLKKISNQAEFIA | |
| | SFYNNDLIKINGELYRVIGVNNDLLNRIEVNMIDITYREYLENMNDKRPPRIIKTIAS | |
| | KTQSIKKYSTDILGNLYEVKSKKHPQIIKKGKRPAATKKAGQAKKKKAS | |
| 274 | MDYKDHDGDYKDHDIDYKDDDDKHGPREGQSDERALLDQLHTLLSNTDATGLEEIDRA | Multipartite |
| | LGIPELVNQGQALEPKQDNGGGGSHEKFPSDLDLDMENGSLECDMESIIRSELMDADG | activator-dSaCas9- |
| | LDFNFDSLGGGGSEGQSDERALLDQLHTLLSNTDATGLEEIDRALGIPELVNQGQALE | NCOA3 FOXO3 |
| | PKQDGGGGSGGGGSGGGGSNPKKKRKVGIHGVPAAKRNYILGLAIGITSVGYGIIDYE | NCOA3 (aa) |
| | TRDVIDAGVRLFKEANVENNEGRRSKRGARRLKRRRRHRIQRVKKLLFDYNLLTDHSE | |
| | LSGINPYEARVKGLSQKLSEEEFSAALLHLAKRRGVHNVNEVEEDTGNELSTKEQISR | |
| | NSKALEEKYVAELQLERLKKDGEVRGSINRFKTSDYVKEAKQLLKVQKAYHQLDQSFI | |
| | DTYIDLLETRRTYYEGPGEGSPFGWKDIKEWYEMLMGHCTYFPEELRSVKYAYNADLY | |
| | NALNDLNNLVITRDENEKLEYYEKFQIIENVFKQKKKPTLKQIAKEILVNEEDIKGYR | |
| | VTSTGKPEFTNLKVYHDIKDITARKEIIENAELLDQIAKILTIYQSSEDIQEELTNLN | |
| | SELTQEEIEQISNLKGYTGTHNLSLKAINLILDELWHTNDNQIAIFNRLKLVPKKVDL | |
| | SQQKEIPTTLVDDFILSPVVKRSFIQSIKVINAIIKKYGLPNDIIIELAREKNSKDAQ | |
| | KMINEMQKRNRQTNERIEEIIRTTGKENAKYLIEKIKLHDMQEGKCLYSLEAIPLEDL | |
| | LNNPFNYEVDHIIPRSVSFDNSFNNKVLVKQEEASKKGNRTPFQYLSSSDSKISYETF | |
| | KKHILNLAKGKGRISKTKKEYLLEERDINRFSVQKDFINRNLVDTRYATRGLMNLLRS | |
| | YFRVNNLDVKVKSINGGFTSFLRRKWKFKKERNKGYKHHAEDALIIANADFIFKEWKK | |
| | LDKAKKVMENQMFEEKQAESMPEIETEQEYKEIFITPHQIKHIKDFKDYKYSHRVDKK | |
| | PNRELINDTLYSTRKDDKGNTLIVNNLNGLYDKDNDKLKKLINKSPEKLLMYHHDPQT | |
| | YQKLKLIMEQYGDEKNPLYKYYEETGNYLTKYSKKDNGPVIKKIKYYGNKLNAHLDIT | |
| | DDYPNSRNKVVKLSLKPYRFDVYLDNGVYKFVTVKNLDVIKKENYYEVNSKCYEEAKK | |
| | LKKISNQAEFIASFYNNDLIKINGELYRVIGVNNDLLNRIEVNMIDITYREYLENMND | |
| | KRPPRIIKTIASKTQSIKKYSTDILGNLYEVKSKKHPQIIKKGKRPAATKKAGQAKKK | |
| | KAS | |
| 275 | MDYKDHDGDYKDHDIDYKDDDDKHGPRWFHGAISREDAENLLESQPLGSFLIRVSHSH | Multipartite |
| | VGYTLSYKAQSSCCHFMVKLLDDGTFMIPGEKVAHTSLDALVTFHNGGGGSHEKFPSD | activator-dSaCas9- |
| | LDLDMFNGSLECDMESIIRSELMDADGLDFNFDSLGGGGSEGQSDERALLDQLHTLLS | HSH2D FOXO3 |
| | NTDATGLEEIDRALGIPELVNQGQALEPKQDGGGGSGGGGSGGGGSNPKKKRKVGIHG | NCOA3 (aa) |
| | VPAAKRNYILGLAIGITSVGYGIIDYETRDVIDAGVRLFKEANVENNEGRRSKRGARR | |
| | LKRRRRHRIQRVKKLLFDYNLLTDHSELSGINPYEARVKGLSQKLSEEEFSAALLHLA | |
| | KRRGVHNVNEVEEDTGNELSTKEQISRNSKALEEKYVAELQLERLKKDGEVRGSINRF | |
| | KTSDYVKEAKQLLKVQKAYHQLDQSFIDTYIDLLETRRTYYEGPGEGSPFGWKDIKEW | |
| | YEMLMGHCTYFPEELRSVKYAYNADLYNALNDLNNLVITRDENEKLEYYEKFQIIENV | |
| | FKQKKKPTLKQIAKEILVNEEDIKGYRVTSTGKPEFTNLKVYHDIKDITARKEIIENA | |
| | ELLDQIAKILTIYQSSEDIQEELTNLNSELTQEEIEQISNLKGYTGTHNLSLKAINLI | |
| | LDELWHTNDNQIAIFNRLKLVPKKVDLSQQKEIPTTLVDDFILSPVVKRSFIQSIKVI | |
| | NAIIKKYGLPNDIIIELAREKNSKDAQKMINEMQKRNRQTNERIEEIIRTTGKENAKY | |
| | LIEKIKLHDMQEGKCLYSLEAIPLEDLLNNPFNYEVDHIIPRSVSFDNSENNKVLVKQ | |
| | EEASKKGNRTPFQYLSSSDSKISYETFKKHILNLAKGKGRISKTKKEYLLEERDINRF | |
| | SVQKDFINRNLVDTRYATRGLMNLLRSYFRVNNLDVKVKSINGGFTSFLRRKWKFKKE | |
| | RNKGYKHHAEDALIIANADFIFKEWKKLDKAKKVMENQMFEEKQAESMPEIETEQEYK | |
| | EIFITPHQIKHIKDFKDYKYSHRVDKKPNRELINDTLYSTRKDDKGNTLIVNNLNGLY | |
| | DKDNDKLKKLINKSPEKLLMYHHDPQTYQKLKLIMEQYGDEKNPLYKYYEETGNYLTK | |
| | YSKKDNGPVIKKIKYYGNKLNAHLDITDDYPNSRNKVVKLSLKPYRFDVYLDNGVYKF | |
| | VTVKNLDVIKKENYYEVNSKCYEEAKKLKKISNQAEFIASFYNNDLIKINGELYRVIG | |
| | VNNDLLNRIEVNMIDITYREYLENMNDKRPPRIIKTIASKTQSIKKYSTDILGNLYEV | |
| | KSKKHPQIIKKGKRPAATKKAGQAKKKKAS | |
| 276 | MDYKDHDGDYKDHDIDYKDDDDKHGPRHEKFPSDLDLDMENGSLECDMESIIRSELMD | Multipartite |
| | ADGLDFNFDSLNGGGGSHEKFPSDLDLDMENGSLECDMESIIRSELMDADGLDENEDS | activator-dSaCas9- |
| | LGGGGSEGQSDERALLDQLHTLLSNTDATGLEEIDRALGIPELVNQGQALEPKQDGGG | FOXO3 FOXO3 |
| | GSGGGGSGGGGSNPKKKRKVGIHGVPAAKRNYILGLAIGITSVGYGIIDYETRDVIDA | NCOA3 (aa) |
| | GVRLFKEANVENNEGRRSKRGARRLKRRRRHRIQRVKKLLFDYNLLTDHSELSGINPY | |
| | EARVKGLSQKLSEEEFSAALLHLAKRRGVHNVNEVEEDTGNELSTKEQISRNSKALEE | |
| | KYVAELQLERLKKDGEVRGSINRFKTSDYVKEAKQLLKVQKAYHQLDQSFIDTYIDLL | |
| | ETRRTYYEGPGEGSPFGWKDIKEWYEMLMGHCTYFPEELRSVKYAYNADLYNALNDLN | |
| | NLVITRDENEKLEYYEKFQIIENVFKQKKKPTLKQIAKEILVNEEDIKGYRVTSTGKP | |
| | EFTNLKVYHDIKDITARKEIIENAELLDQIAKILTIYQSSEDIQEELTNLNSELTQEE | |
| | IEQISNLKGYTGTHNLSLKAINLILDELWHTNDNQIAIFNRLKLVPKKVDLSQQKEIP | |
| | TTLVDDFILSPVVKRSFIQSIKVINAIIKKYGLPNDIIIELAREKNSKDAQKMINEMQ | |
| | KRNRQTNERIEEIIRTTGKENAKYLIEKIKLHDMQEGKCLYSLEAIPLEDLLNNPENY | |
| | EVDHIIPRSVSFDNSFNNKVLVKQEEASKKGNRTPFQYLSSSDSKISYETFKKHILNL | |
| | AKGKGRISKTKKEYLLEERDINRFSVQKDFINRNLVDTRYATRGLMNLLRSYFRVNNL | |
| | DVKVKSINGGFTSFLRRKWKFKKERNKGYKHHAEDALIIANADFIFKEWKKLDKAKKV | |
| | MENQMFEEKQAESMPEIETEQEYKEIFITPHQIKHIKDFKDYKYSHRVDKKPNRELIN | |
| | DTLYSTRKDDKGNTLIVNNLNGLYDKDNDKLKKLINKSPEKLLMYHHDPQTYQKLKLI | |
| | MEQYGDEKNPLYKYYEETGNYLTKYSKKDNGPVIKKIKYYGNKLNAHLDITDDYPNSR | |
| | NKVVKLSLKPYRFDVYLDNGVYKFVTVKNLDVIKKENYYEVNSKCYEEAKKLKKISNQ | |
| | AEFIASFYNNDLIKINGELYRVIGVNNDLLNRIEVNMIDITYREYLENMNDKRPPRII | |
| | KTIASKTQSIKKYSTDILGNLYEVKSKKHPQIIKKGKRPAATKKAGQAKKKKAS | |
| 277 | MDYKDHDGDYKDHDIDYKDDDDKHGPRESPSDEGALLDQLYLALRNEDGLEEIDRALG | Multipartite |
| | IPELVSQSQAVDPEQFNGGGGSHEKFPSDLDLDMENGSLECDMESIIRSELMDADGLD | activator-dSaCas9- |
| | FNFDSLGGGGSEGQSDERALLDQLHTLLSNTDATGLEEIDRALGIPELVNQGQALEPK | NCOA2 FOXO3 |
| | QDGGGGSGGGGSGGGGSNPKKKRKVGIHGVPAAKRNYILGLAIGITSVGYGIIDYETR | NCOA3 (aa) |
| | DVIDAGVRLFKEANVENNEGRRSKRGARRLKRRRRHRIQRVKKLLFDYNLLTDHSELS | |
| | GINPYEARVKGLSQKLSEEEFSAALLHLAKRRGVHNVNEVEEDTGNELSTKEQISRNS | |
| | KALEEKYVAELQLERLKKDGEVRGSINRFKTSDYVKEAKQLLKVQKAYHQLDQSFIDT | |
| | YIDLLETRRTYYEGPGEGSPFGWKDIKEWYEMLMGHCTYFPEELRSVKYAYNADLYNA | |
| | LNDLNNLVITRDENEKLEYYEKFQIIENVFKQKKKPTLKQIAKEILVNEEDIKGYRVT | |
| | STGKPEFTNLKVYHDIKDITARKEIIENAELLDQIAKILTIYQSSEDIQEELTNLNSE | |
| | LTQEEIEQISNLKGYTGTHNLSLKAINLILDELWHTNDNQIAIFNRLKLVPKKVDLSQ | |
| | QKEIPTTLVDDFILSPVVKRSFIQSIKVINAIIKKYGLPNDIIIELAREKNSKDAQKM | |
| | INEMQKRNRQTNERIEEIIRTTGKENAKYLIEKIKLHDMQEGKCLYSLEAIPLEDLLN | |
| | NPFNYEVDHIIPRSVSFDNSFNNKVLVKQEEASKKGNRTPFQYLSSSDSKISYETFKK | |
| | HILNLAKGKGRISKTKKEYLLEERDINRFSVQKDFINRNLVDTRYATRGLMNLLRSYF | |
| | RVNNLDVKVKSINGGFTSFLRRKWKFKKERNKGYKHHAEDALIIANADFIFKEWKKLD | |
| | KAKKVMENQMFEEKQAESMPEIETEQEYKEIFITPHQIKHIKDFKDYKYSHRVDKKPN | |
| | RELINDTLYSTRKDDKGNTLIVNNLNGLYDKDNDKLKKLINKSPEKLLMYHHDPQTYQ | |
| | KLKLIMEQYGDEKNPLYKYYEETGNYLTKYSKKDNGPVIKKIKYYGNKLNAHLDITDD | |
| | YPNSRNKVVKLSLKPYRFDVYLDNGVYKFVTVKNLDVIKKENYYEVNSKCYEEAKKLK | |
| | KISNQAEFIASFYNNDLIKINGELYRVIGVNNDLLNRIEVNMIDITYREYLENMNDKR | |
| | PPRIIKTIASKTQSIKKYSTDILGNLYEVKSKKHPQIIKKGKRPAATKKAGQAKKKKA | |
| | S | |
| 278 | MDYKDHDGDYKDHDIDYKDDDDKHGPRAYTDELVELHRRLMALRERNVLQQIVNLIEE | Multipartite |
| | TGHFNVTNTTFDFDLESLDETTVRKLQSCLENGGGGSHEKFPSDLDLDMENGSLECDM | activator-dSaCas9- |
| | ESIIRSELMDADGLDFNFDSLGGGGSEGQSDERALLDQLHTLLSNTDATGLEEIDRAL | ENL FOXO3 |
| | GIPELVNQGQALEPKQDGGGGSGGGGSGGGGSNPKKKRKVGIHGVPAAKRNYILGLAI | NCOA3 (aa) |
| | GITSVGYGIIDYETRDVIDAGVRLFKEANVENNEGRRSKRGARRLKRRRRHRIQRVKK | |
| | LLFDYNLLTDHSELSGINPYEARVKGLSQKLSEEEFSAALLHLAKRRGVHNVNEVEED | |
| | TGNELSTKEQISRNSKALEEKYVAELQLERLKKDGEVRGSINRFKTSDYVKEAKQLLK | |
| | VQKAYHQLDQSFIDTYIDLLETRRTYYEGPGEGSPFGWKDIKEWYEMLMGHCTYFPEE | |
| | LRSVKYAYNADLYNALNDLNNLVITRDENEKLEYYEKFQIIENVFKQKKKPTLKQIAK | |
| | EILVNEEDIKGYRVTSTGKPEFTNLKVYHDIKDITARKEIIENAELLDQIAKILTIYQ | |
| | SSEDIQEELTNLNSELTQEEIEQISNLKGYTGTHNLSLKAINLILDELWHTNDNQIAI | |
| | FNRLKLVPKKVDLSQQKEIPTTLVDDFILSPVVKRSFIQSIKVINAIIKKYGLPNDII | |
| | IELAREKNSKDAQKMINEMQKRNRQTNERIEEIIRTTGKENAKYLIEKIKLHDMQEGK | |
| | CLYSLEAIPLEDLLNNPFNYEVDHIIPRSVSFDNSFNNKVLVKQEEASKKGNRTPFQY | |
| | LSSSDSKISYETFKKHILNLAKGKGRISKTKKEYLLEERDINRFSVQKDFINRNLVDT | |
| | RYATRGLMNLLRSYFRVNNLDVKVKSINGGFTSFLRRKWKFKKERNKGYKHHAEDALI | |
| | IANADFIFKEWKKLDKAKKVMENQMFEEKQAESMPEIETEQEYKEIFITPHQIKHIKD | |
| | FKDYKYSHRVDKKPNRELINDTLYSTRKDDKGNTLIVNNLNGLYDKDNDKLKKLINKS | |
| | PEKLLMYHHDPQTYQKLKLIMEQYGDEKNPLYKYYEETGNYLTKYSKKDNGPVIKKIK | |
| | YYGNKLNAHLDITDDYPNSRNKVVKLSLKPYRFDVYLDNGVYKFVTVKNLDVIKKENY | |
| | YEVNSKCYEEAKKLKKISNQAEFIASFYNNDLIKINGELYRVIGVNNDLLNRIEVNMI | |
| | DITYREYLENMNDKRPPRIIKTIASKTQSIKKYSTDILGNLYEVKSKKHPQIIKKGKR | |
| | PAATKKAGQAKKKKAS | |
| 279 | ATGACCGAGTACAAGCCCACGGTGCGCCTCGCCACCCGCGACGACGTCCCCAGGGCCG | Puromycin |
| | TACGCACCCTCGCCGCCGCGTTCGCCGACTACCCCGCCACGCGCCACACCGTCGATCC | resistance gene (nt) |
| | GGACCGCCACATCGAGCGGGTCACCGAGCTGCAAGAACTCTTCCTCACGCGCGTCGGG | |
| | CTCGACATCGGCAAGGTGTGGGTCGCGGACGACGGCGCCGCGGTGGCGGTCTGGACCA | |
| | CGCCGGAGAGCGTCGAAGCGGGGGCGGTGTTCGCCGAGATCGGCCCGCGCATGGCCGA | |
| | GTTGAGCGGTTCCCGGCTGGCCGCGCAGCAACAGATGGAAGGCCTCCTGGCGCCGCAC | |
| | CGGCCCAAGGAGCCCGCGTGGTTCCTGGCCACCGTCGGCGTGTCGCCCGACCACCAGG | |
| | GCAAGGGTCTGGGCAGCGCCGTCGTGCTCCCCGGAGTGGAGGCGGCCGAGCGCGCCGG | |
| | GGTGCCCGCCTTCCTGGAGACCTCCGCGCCCCGCAACCTCCCCTTCTACGAGCGGCTC | |
| | GGCTTCACCGTCACCGCCGACGTCGAGGTGCCCGAAGGACCGCGCACCTGGTGCATGA | |
| | CCCGCAAGCCCGGTGCCTGA | |
| 280 | MTEYKPTVRLATRDDVPRAVRTLAAAFADYPATRHTVDPDRHIERVTELQELFLTRVG | Puromycin |
| | LDIGKVWVADDGAAVAVWTTPESVEAGAVFAEIGPRMAELSGSRLAAQQQMEGLLAPH | resistance gene (aa) |
| | RPKEPAWFLATVGVSPDHQGKGLGSAVVLPGVEAAERAGVPAFLETSAPRNLPFYERL | |
| | GFTVTADVEVPEGPRTWCMTRKPGA | |
| 281 | PCGICTNEVNDDQDAILCEASCQKWFHRICTGMTETAYGLLTAEASAVWGCDTCMADN | Bipartite activator- |
| | GGGGSEGQSDERALLDQLHTLLSNTDATGLEEIDRALGIPELVNQGQALEPKQDGGGG | PYGO1 NCOA3 |
| | SGGGGSGGGGS | (aa) |
| 282 | DIDECASNPCRKGATCINGVNGFRCICPEGPHHPSCYNGGGGSEGQSDERALLDQLHT | Bipartite activator- |
| | LLSNTDATGLEEIDRALGIPELVNQGQALEPKQDGGGGSGGGGSGGGGS | NOTCH2 NCOA3 |
| | | (aa) |
| 283 | EGQSDERALLDQLHTLLSNTDATGLEEIDRALGIPELVNQGQALEPKQDGGGGSEGQS | Bipartite activator- |
| | DERALLDQLHTLLSNTDATGLEEIDRALGIPELVNQGQALEPKQDGGGGSGGGGSGGG | NCOA3 NCOA3 |
| | GS | (aa) |
| 284 | WFHGAISREDAENLLESQPLGSFLIRVSHSHVGYTLSYKAQSSCCHFMVKLLDDGTEM | Bipartite activator- |
| | IPGEKVAHTSLDALVTFHNGGGGSEGQSDERALLDQLHTLLSNTDATGLEEIDRALGI | HSH2D NCOA3 |
| | PELVNQGQALEPKQDGGGGSGGGGSGGGGS | (aa) |
| 285 | HEKFPSDLDLDMENGSLECDMESIIRSELMDADGLDFNFDSLGGGGSEGQSDERALLD | Bipartite activator- |
| | QLHTLLSNTDATGLEEIDRALGIPELVNQGQALEPKQDGGGGSGGGGSGGGGS | FOXO3 NCOA3 |
| | | (aa) |
| 286 | ESPSDEGALLDQLYLALRNEDGLEEIDRALGIPELVSQSQAVDPEQENGGGGSEGQSD | Bipartite activator- |
| | ERALLDQLHTLLSNTDATGLEEIDRALGIPELVNQGQALEPKQDGGGGSGGGGSGGGG | NCOA2 NCOA3 |
| | S | (aa) |
| 287 | AYTDELVELHRRLMALRERNVLQQIVNLIEETGHFNVTNTTFDFDLESLDETTVRKLQ | Bipartite activator- |
| | SCLENGGGGSEGQSDERALLDQLHTLLSNTDATGLEEIDRALGIPELVNQGQALEPKQ | ENL NCOA3 (aa) |
| | DGGGGSGGGGSGGGGS | |
| 288 | PCGICTNEVNDDQDAILCEASCQKWFHRICTGMTETAYGLLTAEASAVWGCDTCMADN | Bipartite activator- |
| | GGGGSHEKFPSDLDLDMENGSLECDMESIIRSELMDADGLDFNFDSLGGGGSGGGGSG | PYGO1 FOXO3 |
| | GGGS | (aa) |
| 289 | DIDECASNPCRKGATCINGVNGFRCICPEGPHHPSCYNGGGGSHEKFPSDLDLDMENG | Bipartite activator- |
| | SLECDMESIIRSELMDADGLDFNFDSLGGGGSGGGGSGGGGS | NOTCH2 FOXO3 |
| | | (aa) |
| 290 | EGQSDERALLDQLHTLLSNTDATGLEEIDRALGIPELVNQGQALEPKQDNGGGGSHEK | Bipartite activator- |
| | FPSDLDLDMENGSLECDMESIIRSELMDADGLDFNFDSLGGGGSGGGGSGGGGS | NCOA3 FOXO3 |
| | | (aa) |
| 291 | WFHGAISREDAENLLESQPLGSFLIRVSHSHVGYTLSYKAQSSCCHFMVKLLDDGTEM | Bipartite activator- |
| | IPGEKVAHTSLDALVTFHNGGGGSHEKFPSDLDLDMENGSLECDMESIIRSELMDADG | HSH2D FOXO3 |
| | LDFNFDSLGGGGSGGGGSGGGGS | (aa) |
| 292 | HEKFPSDLDLDMENGSLECDMESIIRSELMDADGLDFNFDSLNGGGGSHEKFPSDLDL | Bipartite activator- |
| | DMFNGSLECDMESIIRSELMDADGLDFNFDSLGGGGSGGGGSGGGGS | FOXO3 FOXO3 |
| | | (aa) |
| 293 | ESPSDEGALLDQLYLALRNEDGLEEIDRALGIPELVSQSQAVDPEQENGGGGSHEKFP | Bipartite activator- |
| | SDLDLDMENGSLECDMESIIRSELMDADGLDFNFDSLGGGGSGGGGSGGGGS | NCOA2 FOXO3 |
| | | (aa) |
| 294 | AYTDELVELHRRLMALRERNVLQQIVNLIEETGHENVTNTTFDFDLFSLDETTVRKLQ | Bipartite activator- |
| | SCLENGGGGSHEKFPSDLDLDMENGSLECDMESIIRSELMDADGLDFNFDSLGGGGSG | ENL FOXO3 (aa) |
| | GGGSGGGGS | |
| 295 | PCGICTNEVNDDQDAILCEASCQKWFHRICTGMTETAYGLLTAEASAVWGCDTCMADN | Tripartite activator- |
| | GGGGSHEKFPSDLDLDMENGSLECDMESIIRSELMDADGLDFNFDSLGGGGSEGQSDE | PYGO1 FOXO3 |
| | RALLDQLHTLLSNTDATGLEEIDRALGIPELVNQGQALEPKQDGGGGSGGGGSGGGGS | NCOA3 (aa) |
| 296 | DIDECASNPCRKGATCINGVNGFRCICPEGPHHPSCYNGGGGSHEKFPSDLDLDMENG | Tripartite activator- |
| | SLECDMESIIRSELMDADGLDENEDSLGGGGSEGQSDERALLDQLHTLLSNTDATGLE | NOTCH2 FOXO3 |
| | EIDRALGIPELVNQGQALEPKQDGGGGSGGGGSGGGGS | NCOA3 (aa) |
| 297 | EGQSDERALLDQLHTLLSNTDATGLEEIDRALGIPELVNQGQALEPKQDNGGGGSHEK | Tripartite activator- |
| | FPSDLDLDMENGSLECDMESIIRSELMDADGLDENFDSLGGGGSEGQSDERALLDQLH | NCOA3 FOXO3 |
| | TLLSNTDATGLEEIDRALGIPELVNQGQALEPKQDGGGGSGGGGSGGGGS | NCOA3 (aa) |
| 298 | WFHGAISREDAENLLESQPLGSFLIRVSHSHVGYTLSYKAQSSCCHFMVKLLDDGTEM | Tripartite activator- |
| | IPGEKVAHTSLDALVTFHNGGGGSHEKFPSDLDLDMENGSLECDMESIIRSELMDADG | HSH2D FOXO3 |
| | LDFNFDSLGGGGSEGQSDERALLDQLHTLLSNTDATGLEEIDRALGIPELVNQGQALE | NCOA3 (aa) |
| | PKQDGGGGSGGGGSGGGGS | |
| 299 | HEKFPSDLDLDMENGSLECDMESIIRSELMDADGLDFNFDSLNGGGGSHEKFPSDLDL | Tripartite activator- |
| | DMFNGSLECDMESIIRSELMDADGLDFNFDSLGGGGSEGQSDERALLDQLHTLLSNTD | FOXO3 FOXO3 |
| | ATGLEEIDRALGIPELVNQGQALEPKQDGGGGSGGGGSGGGGS | NCOA3 (aa) |
| 300 | ESPSDEGALLDQLYLALRNEDGLEEIDRALGIPELVSQSQAVDPEQFNGGGGSHEKFP | Tripartite activator- |
| | SDLDLDMENGSLECDMESIIRSELMDADGLDFNFDSLGGGGSEGQSDERALLDQLHTL | NCOA2 FOXO3 |
| | LSNTDATGLEEIDRALGIPELVNQGQALEPKQDGGGGSGGGGSGGGGS | NCOA3 (aa) |
| 301 | AYTDELVELHRRLMALRERNVLQQIVNLIEETGHENVTNTTEDEDLESLDETTVRKLQ | Tripartite activator- |
| | SCLENGGGGSHEKFPSDLDLDMENGSLECDMESIIRSELMDADGLDENEDSLGGGGSE | ENL FOXO3 |
| | GQSDERALLDQLHTLLSNTDATGLEEIDRALGIPELVNQGQALEPKQDGGGGSGGGGS | NCOA3 (aa) |
| | GGGGS | |
| 302 | NNNNGNNN | N. meningitidis |
| | | Cas9 PAM |
| 303 | AKNTITKTLKLRIVRPYNSAEVEKIVADEKNNREKIALEKNKDKVKEACSKHLKVAAY | CasMini |
| | CTTQVERNACLFCKARKLDDKFYQKLRGQFPDAVFWQEISEIFRQLQKQAAEIYNQSL | Cas12f/D326A/ |
| | IELYYEIFIKGKGIANASSVEHYLSRVCYRRAAELFKNAAIASGLRSKIKSNFRLKEL | D510A/D143R/T147R/ |
| | KNMKSGLPTTKSDNFPIPLVKQKGGQYTGFEISNHNSDFIIKIPFGRWQVKKEIDKYR | K330R/E528R (aa) |
| | PWEKFDFEQVQKSPKPISLLLSTQRRKRNKGWSKDEGTEAEIKKVMNGDYQTSYIEVK | |
| | RGSKICEKSAWMLNLSIDVPKIDKGVDPSIIGGIAVGVRSPLVCAINNAFSRYSISDN | |
| | DLFHFNKKMFARRRILLKKNRHKRAGHGAKNKLKPITILTEKSERFRKKLIERWACEI | |
| | ADFFIKNKVGTVQMENLESMKRKEDSYFNIRLRGFWPYAEMQNKIEFKLKQYGIEIRK | |
| | VAPNNTSKTCSKCGHLNNYENFEYRKKNKFPHFKCEKCNFKENAAYNAALNISNPKLK | |
| | STKERP | |
| 304 | TBN | PAM-CasPhi |
| 305 | TTTN | PAM-UnlCas12fl |
| 306 | NNNNACAC | PAM-CjCas9 |
| 307 | GGTT | PAM-Cas12k |
| 308 | TTTR | PAM-Cas12f |
| 309 | MKRNYILGLAIGITSVGYGIIDYETRDVIDAGVRLFKEANVENNEGRRSKRGARRLKR | dSaCas9 (aa) |
| | RRRHRIQRVKKLLFDYNLLTDHSELSGINPYEARVKGLSQKLSEEEFSAALLHLAKRR | |
| | GVHNVNEVEEDTGNELSTKEQISRNSKALEEKYVAELQLERLKKDGEVRGSINRFKTS | |
| | DYVKEAKQLLKVQKAYHQLDQSFIDTYIDLLETRRTYYEGPGEGSPFGWKDIKEWYEM | |
| | LMGHCTYFPEELRSVKYAYNADLYNALNDLNNLVITRDENEKLEYYEKFQIIENVEKQ | |
| | KKKPTLKQIAKEILVNEEDIKGYRVTSTGKPEFTNLKVYHDIKDITARKEIIENAELL | |
| | DQIAKILTIYQSSEDIQEELTNLNSELTQEEIEQISNLKGYTGTHNLSLKAINLILDE | |
| | LWHTNDNQIAIFNRLKLVPKKVDLSQQKEIPTTLVDDFILSPVVKRSFIQSIKVINAI | |
| | IKKYGLPNDIIIELAREKNSKDAQKMINEMQKRNRQTNERIEEIIRTTGKENAKYLIE | |
| | KIKLHDMQEGKCLYSLEAIPLEDLLNNPFNYEVDHIIPRSVSEDNSENNKVLVKQEEA | |
| | SKKGNRTPFQYLSSSDSKISYETFKKHILNLAKGKGRISKTKKEYLLEERDINRFSVQ | |
| | KDFINRNLVDTRYATRGLMNLLRSYFRVNNLDVKVKSINGGFTSFLRRKWKFKKERNK | |
| | GYKHHAEDALIIANADFIFKEWKKLDKAKKVMENQMFEEKQAESMPEIETEQEYKEIF | |
| | ITPHQIKHIKDFKDYKYSHRVDKKPNRELINDTLYSTRKDDKGNTLIVNNLNGLYDKD | |
| | NDKLKKLINKSPEKLLMYHHDPQTYQKLKLIMEQYGDEKNPLYKYYEETGNYLTKYSK | |
| | KDNGPVIKKIKYYGNKLNAHLDITDDYPNSRNKVVKLSLKPYRFDVYLDNGVYKFVTV | |
| | KNLDVIKKENYYEVNSKCYEEAKKLKKISNQAEFIASFYNNDLIKINGELYRVIGVNN | |
| | DLLNRIEVNMIDITYREYLENMNDKRPPRIIKTIASKTQSIKKYSTDILGNLYEVKSK | |
| | KHPQIIKKG | |
| 310 | MDKKYSIGLAIGTNSVGWAVITDEYKVPSKKFKVLGNTDRHSIKKNLIGALLFDSGET | dSpCas9 (aa) |
| | AEATRLKRTARRRYTRRKNRICYLQEIFSNEMAKVDDSFFHRLEESFLVEEDKKHERH | |
| | PIFGNIVDEVAYHEKYPTIYHLRKKLVDSTDKADLRLIYLALAHMIKERGHFLIEGDL | |
| | NPDNSDVDKLFIQLVQTYNQLFEENPINASGVDAKAILSARLSKSRRLENLIAQLPGE | |
| | KKNGLFGNLIALSLGLTPNFKSNEDLAEDAKLQLSKDTYDDDLDNLLAQIGDQYADLE | |
| | LAAKNLSDAILLSDILRVNTEITKAPLSASMIKRYDEHHQDLTLLKALVRQQLPEKYK | |
| | EIFFDQSKNGYAGYIDGGASQEEFYKFIKPILEKMDGTEELLVKLNREDLLRKQRTED | |
| | NGSIPHQIHLGELHAILRRQEDFYPFLKDNREKIEKILTFRIPYYVGPLARGNSRFAW | |
| | MTRKSEETITPWNFEEVVDKGASAQSFIERMTNFDKNLPNEKVLPKHSLLYEYFTVYN | |
| | ELTKVKYVTEGMRKPAFLSGEQKKAIVDLLFKTNRKVTVKQLKEDYFKKIECFDSVEI | |
| | SGVEDRFNASLGTYHDLLKIIKDKDFLDNEENEDILEDIVLTLTLFEDREMIEERLKT | |
| | YAHLFDDKVMKQLKRRRYTGWGRLSRKLINGIRDKQSGKTILDFLKSDGFANRNEMQL | |
| | IHDDSLTFKEDIQKAQVSGQGDSLHEHIANLAGSPAIKKGILQTVKVVDELVKVMGRH | |
| | KPENIVIEMARENQTTQKGQKNSRERMKRIEEGIKELGSQILKEHPVENTQLQNEKLY | |
| | LYYLQNGRDMYVDQELDINRLSDYDVDAIVPQSFLKDDSIDNKVLTRSDKNRGKSDNV | |
| | PSEEVVKKMKNYWRQLLNAKLITQRKFDNLTKAERGGLSELDKAGFIKRQLVETRQIT | |
| | KHVAQILDSRMNTKYDENDKLIREVKVITLKSKLVSDFRKDFQFYKVREINNYHHAHD | |
| | AYLNAVVGTALIKKYPKLESEFVYGDYKVYDVRKMIAKSEQEIGKATAKYFFYSNIMN | |
| | FFKTEITLANGEIRKRPLIETNGETGEIVWDKGRDFATVRKVLSMPQVNIVKKTEVQT | |
| | GGFSKESILPKRNSDKLIARKKDWDPKKYGGFDSPTVAYSVLVVAKVEKGKSKKLKSV | |
| | KELLGITIMERSSFEKNPIDFLEAKGYKEVKKDLIIKLPKYSLFELENGRKRMLASAG | |
| | ELQKGNELALPSKYVNFLYLASHYEKLKGSPEDNEQKQLFVEQHKHYLDEIIEQISEF | |
| | SKRVILADANLDKVLSAYNKHRDKPIREQAENIIHLFTLTNLGAPAAFKYFDTTIDRK | |
| | RYTSTKEVLDATLIHQSITGLYETRIDLSQLGGD | |
| 311 | gaaacaccguaauuucuacucuuguagau | dAsCas12a gRNA |
| | | scaffold |
| 312 | CAACGAUUGCCCCUCACGAGGGGAC | dCasPhi-2 gRNA |
| | | scaffold |
| 313 | ACCGCUUCACUUAGAGUGAAGGUGGGCUGCUUGCAUCAGCCUAAUGUCGAGAAGUGCU | Un1Cas12fl gRNA |
| | UUCUUCGGAAAGUAACCCUCGAAACAAAGAAA | scaffold |
| 314 | GGAAUGAAC | Un1Cas12fl gRNA |
| | | scaffold |
| 315 | UUUUAUUUU | Un1Cas12fl gRNA |
| | | scaffold |
| 316 | GCGGUUUUAGGGGAUUGUAACCCCGCAGAGUCCCGCAAACUCUUUAUUUUAGUCCCUU | CjCas9 gRNA |
| | UUCAGGGACUAAAAC | scaffold |
| 317 | AUAUUAAUAGCGCCGCAAUUCAUGCUGCUUGCAGCCUCUGAAUUUUGUUAAAUGAGGG | Cas12k gRNA |
| | UUAGUUUGACUGUAUAAAUACAGUCUUGCUUUCUGACCCUGGUAGCUGCUCACCCUGA | scaffold |
| | UGCUGCUGUCAAUAGACAGGAUAGGUGCGCUCCCAGCAAUAAGGGCGCGGAUGUACUG | |
| | CUGUAGUGGCUACUGAAUCACCCCCGAUCAAGGGGGAACCCUCCAAAAGGUGGGUUGA | |
| | AAG | |
| 318 | GGGCUUCACUGAUAAAGUGGAGAACCGCUUCACCAAAAGCUGUCCCUUAGGGGAUUAG | CasMini gRNA |
| | AACUUGAGUGAAGGUGGGCUGCUUGCAUCAGCCUAAUGUCGAGAAGUGCUUUCUUCGG | scaffold |
| | AAAGUAACCCUCGAAACAAAUUCAUUUGAAUGAAGGAAUGCAAC | |
| 319 | AAGTAAGGCAAAAAGATTGC | gRNA V spacer |
| 320 | CAATTTTACTCCTTAGGGGA | gRNA W spacer |
| 321 | GAGAGCAGATACTACTGCCA | gRNA X spacer |
| 322 | TGGAGACCAAGAATGCTGCT | gRNA Y spacer |
| 323 | TGATTTTGTGATCTTGCGAA | gRNA Z spacer |
| 324 | TAACATGCTGTACAGGTTTA | gRNA AA spacer |
| 325 | CTAGACCATCTAGCTTTGTG | gRNA AB spacer |
| 326 | TCTCCACCCACTAGATGGCA | gRNA AC spacer |
| 327 | MTQFEGFTNLYQVSKTLRFELIPQGKTLKHIQEQGFIEEDKARNDHYKELKPIIDRIY | dAsCas12a |
| | KTYADQCLQLVQLDWENLSAAIDSYRKEKTEETRNALIEEQATYRNAIHDYFIGRTDN | (dAsCpf1) (aa) |
| | LTDAINKRHAEIYKGLFKAELFNGKVLKQLGTVTTTEHENALLRSFDKFTTYFSGFYE | |
| | NRKNVFSAEDISTAIPHRIVQDNFPKFKENCHIFTRLITAVPSLREHFENVKKAIGIF | |
| | VSTSIEEVFSFPFYNQLLTQTQIDLYNQLLGGISREAGTEKIKGLNEVLNLAIQKNDE | |
| | TAHIIASLPHRFIPLFKQILSDRNTLSFILEEFKSDEEVIQSFCKYKTLLRNENVLET | |
| | AEALFNELNSIDLTHIFISHKKLETISSALCDHWDTLRNALYERRISELTGKITKSAK | |
| | EKVQRSLKHEDINLQEIISAAGKELSEAFKQKTSEILSHAHAALDQPLPTTLKKQEEK | |
| | EILKSQLDSLLGLYHLLDWFAVDESNEVDPEFSARLTGIKLEMEPSLSFYNKARNYAT | |
| | KKPYSVEKFKLNFQMPTLASGWDVNKEKNNGAILFVKNGLYYLGIMPKQKGRYKALSF | |
| | EPTEKTSEGFDKMYYDYFPDAAKMIPKCSTQLKAVTAHFQTHTTPILLSNNFIEPLEI | |
| | TKEIYDLNNPEKEPKKFQTAYAKKTGDQKGYREALCKWIDFTRDFLSKYTKTTSIDLS | |
| | SLRPSSQYKDLGEYYAELNPLLYHISFQRIAEKEIMDAVETGKLYLFQIYNKDFAKGH | |
| | HGKPNLHTLYWTGLFSPENLAKTSIKLNGQAELFYRPKSRMKRMAHRLGEKMLNKKLK | |
| | DQKTPIPDTLYQELYDYVNHRLSHDLSDEARALLPNVITKEVSHEIIKDRRFTSDKFF | |
| | FHVPITLNYQAANSPSKFNQRVNAYLKEHPETPIIGIARGERNLIYITVIDSTGKILE | |
| | QRSLNTIQQFDYQKKLDNREKERVAARQAWSVVGTIKDLKQGYLSQVIHEIVDLMIHY | |
| | QAVVVLANLNFGFKSKRTGIAEKAVYQQFEKMLIDKLNCLVLKDYPAEKVGGVLNPYQ | |
| | LTDQFTSFAKMGTQSGFLFYVPAPYTSKIDPLTGFVDPFVWKTIKNHESRKHFLEGED | |
| | FLHYDVKTGDFILHFKMNRNLSFQRGLPGFMPAWDIVFEKNETQFDAKGTPFIAGKRI | |
| | VPVIENHRFTGRYRDLYPANELIALLEEKGIVERDGSNILPKLLENDDSHAIDTMVAL | |
| | IRSVLQMRNSNAATGEDYINSPVRDLNGVCFDSRFQNPEWPMDADANGAYHIALKGQL | |
| | LLNHLKESKDLKLQNGISNQDWLAYIQELRN | |
| 328 | TQFEGFTNLYQVSKTLRFELIPQGKTLKHIQEQGFIEEDKARNDHYKELKPIIDRIYK | dAsCas12a |
| | TYADQCLQLVQLDWENLSAAIDSYRKEKTEETRNALIEEQATYRNAIHDYFIGRTDNL | (dAsCpf1) (aa) |
| | TDAINKRHAEIYKGLFKAELFNGKVLKQLGTVTTTEHENALLRSFDKFTTYFSGFYEN | |
| | RKNVESAEDISTAIPHRIVQDNFPKFKENCHIFTRLITAVPSLREHFENVKKAIGIFV | |
| | STSIEEVFSFPFYNQLLTQTQIDLYNQLLGGISREAGTEKIKGLNEVLNLAIQKNDET | |
| | AHIIASLPHRFIPLFKQILSDRNTLSFILEEFKSDEEVIQSFCKYKTLLRNENVLETA | |
| | EALFNELNSIDLTHIFISHKKLETISSALCDHWDTLRNALYERRISELTGKITKSAKE | |
| | KVQRSLKHEDINLQEIISAAGKELSEAFKQKTSEILSHAHAALDQPLPTTLKKQEEKE | |
| | ILKSQLDSLLGLYHLLDWFAVDESNEVDPEFSARLTGIKLEMEPSLSFYNKARNYATK | |
| | KPYSVEKFKLNFQMPTLASGWDVNKEKNNGAILFVKNGLYYLGIMPKQKGRYKALSFE | |
| | PTEKTSEGFDKMYYDYFPDAAKMIPKCSTQLKAVTAHFQTHTTPILLSNNFIEPLEIT | |
| | KEIYDLNNPEKEPKKFQTAYAKKTGDQKGYREALCKWIDFTRDELSKYTKTTSIDLSS | |
| | LRPSSQYKDLGEYYAELNPLLYHISFQRIAEKEIMDAVETGKLYLFQIYNKDFAKGHH | |
| | GKPNLHTLYWTGLFSPENLAKTSIKLNGQAELFYRPKSRMKRMAHRLGEKMLNKKLKD | |
| | QKTPIPDTLYQELYDYVNHRLSHDLSDEARALLPNVITKEVSHEIIKDRRFTSDKFFF | |
| | HVPITLNYQAANSPSKFNQRVNAYLKEHPETPIIGIARGERNLIYITVIDSTGKILEQ | |
| | RSLNTIQQFDYQKKLDNREKERVAARQAWSVVGTIKDLKQGYLSQVIHEIVDLMIHYQ | |
| | AVVVLANLNFGFKSKRTGIAEKAVYQQFEKMLIDKLNCLVLKDYPAEKVGGVLNPYQL | |
| | TDQFTSFAKMGTQSGFLFYVPAPYTSKIDPLTGFVDPFVWKTIKNHESRKHFLEGFDF | |
| | LHYDVKTGDFILHFKMNRNLSFQRGLPGFMPAWDIVFEKNETQFDAKGTPFIAGKRIV | |
| | PVIENHRFTGRYRDLYPANELIALLEEKGIVERDGSNILPKLLENDDSHAIDTMVALI | |
| | RSVLQMRNSNAATGEDYINSPVRDLNGVCFDSRFQNPEWPMDADANGAYHIALKGQLL | |
| | LNHLKESKDLKLQNGISNQDWLAYIQELRN | |
| 329 | MTQFEGFTNLYQVSKTLRFELIPQGKTLKHIQEQGFIEEDKARNDHYKELKPIIDRIY | WT AsCas12a |
| | KTYADQCLQLVQLDWENLSAAIDSYRKEKTEETRNALIEEQATYRNAIHDYFIGRTDN | (AsCpf1) (aa) |
| | LTDAINKRHAEIYKGLFKAELFNGKVLKQLGTVTTTEHENALLRSFDKFTTYFSGFYE | |
| | NRKNVFSAEDISTAIPHRIVQDNFPKFKENCHIFTRLITAVPSLREHFENVKKAIGIF | |
| | VSTSIEEVFSFPFYNQLLTQTQIDLYNQLLGGISREAGTEKIKGLNEVLNLAIQKNDE | |
| | TAHIIASLPHRFIPLFKQILSDRNTLSFILEEFKSDEEVIQSFCKYKTLLRNENVLET | |
| | AEALFNELNSIDLTHIFISHKKLETISSALCDHWDTLRNALYERRISELTGKITKSAK | |
| | EKVQRSLKHEDINLQEIISAAGKELSEAFKQKTSEILSHAHAALDQPLPTTLKKQEEK | |
| | EILKSQLDSLLGLYHLLDWFAVDESNEVDPEFSARLTGIKLEMEPSLSFYNKARNYAT | |
| | KKPYSVEKFKLNFQMPTLASGWDVNKEKNNGAILFVKNGLYYLGIMPKQKGRYKALSF | |
| | EPTEKTSEGFDKMYYDYFPDAAKMIPKCSTQLKAVTAHFQTHTTPILLSNNFIEPLEI | |
| | TKEIYDLNNPEKEPKKFQTAYAKKTGDQKGYREALCKWIDFTRDELSKYTKTTSIDLS | |
| | SLRPSSQYKDLGEYYAELNPLLYHISFQRIAEKEIMDAVETGKLYLFQIYNKDFAKGH | |
| | HGKPNLHTLYWTGLFSPENLAKTSIKLNGQAELFYRPKSRMKRMAHRLGEKMLNKKLK | |
| | DQKTPIPDTLYQELYDYVNHRLSHDLSDEARALLPNVITKEVSHEIIKDRRFTSDKFF | |
| | FHVPITLNYQAANSPSKENQRVNAYLKEHPETPIIGIDRGERNLIYITVIDSTGKILE | |
| | QRSLNTIQQFDYQKKLDNREKERVAARQAWSVVGTIKDLKQGYLSQVIHEIVDLMIHY | |
| | QAVVVLENLNFGFKSKRTGIAEKAVYQQFEKMLIDKLNCLVLKDYPAEKVGGVLNPYQ | |
| | LTDQFTSFAKMGTQSGFLFYVPAPYTSKIDPLTGFVDPFVWKTIKNHESRKHFLEGED | |
| | FLHYDVKTGDFILHFKMNRNLSFQRGLPGFMPAWDIVFEKNETQFDAKGTPFIAGKRI | |
| | VPVIENHRFTGRYRDLYPANELIALLEEKGIVERDGSNILPKLLENDDSHAIDTMVAL | |
| | IRSVLQMRNSNAATGEDYINSPVRDLNGVCFDSRFQNPEWPMDADANGAYHIALKGQL | |
| | LLNHLKESKDLKLQNGISNQDWLAYIQELRN | |
| 330 | TQFEGFTNLYQVSKTLRFELIPQGKTLKHIQEQGFIEEDKARNDHYKELKPIIDRIYK | WT AsCas12a |
| | TYADQCLQLVQLDWENLSAAIDSYRKEKTEETRNALIEEQATYRNAIHDYFIGRTDNL | (AsCpf1) (aa) |
| | TDAINKRHAEIYKGLFKAELFNGKVLKQLGTVTTTEHENALLRSFDKFTTYFSGFYEN | |
| | RKNVFSAEDISTAIPHRIVQDNFPKFKENCHIFTRLITAVPSLREHFENVKKAIGIFV | |
| | STSIEEVFSFPFYNQLLTQTQIDLYNQLLGGISREAGTEKIKGLNEVLNLAIQKNDET | |
| | AHIIASLPHRFIPLFKQILSDRNTLSFILEEFKSDEEVIQSFCKYKTLLRNENVLETA | |
| | EALFNELNSIDLTHIFISHKKLETISSALCDHWDTLRNALYERRISELTGKITKSAKE | |
| | KVQRSLKHEDINLQEIISAAGKELSEAFKQKTSEILSHAHAALDQPLPTTLKKQEEKE | |
| | ILKSQLDSLLGLYHLLDWFAVDESNEVDPEFSARLTGIKLEMEPSLSFYNKARNYATK | |
| | KPYSVEKFKLNFQMPTLASGWDVNKEKNNGAILFVKNGLYYLGIMPKQKGRYKALSFE | |
| | PTEKTSEGFDKMYYDYFPDAAKMIPKCSTQLKAVTAHFQTHTTPILLSNNFIEPLEIT | |
| | KEIYDLNNPEKEPKKFQTAYAKKTGDQKGYREALCKWIDFTRDELSKYTKTTSIDLSS | |
| | LRPSSQYKDLGEYYAELNPLLYHISFQRIAEKEIMDAVETGKLYLFQIYNKDFAKGHH | |
| | GKPNLHTLYWTGLFSPENLAKTSIKLNGQAELFYRPKSRMKRMAHRLGEKMLNKKLKD | |
| | QKTPIPDTLYQELYDYVNHRLSHDLSDEARALLPNVITKEVSHEIIKDRRFTSDKFFF | |
| | HVPITLNYQAANSPSKFNQRVNAYLKEHPETPIIGIDRGERNLIYITVIDSTGKILEQ | |
| | RSLNTIQQFDYQKKLDNREKERVAARQAWSVVGTIKDLKQGYLSQVIHEIVDLMIHYQ | |
| | AVVVLENLNFGFKSKRTGIAEKAVYQQFEKMLIDKLNCLVLKDYPAEKVGGVLNPYQL | |
| | TDQFTSFAKMGTQSGFLFYVPAPYTSKIDPLTGFVDPFVWKTIKNHESRKHFLEGFDF | |
| | LHYDVKTGDFILHFKMNRNLSFQRGLPGFMPAWDIVFEKNETQFDAKGTPFIAGKRIV | |
| | PVIENHRFTGRYRDLYPANELIALLEEKGIVERDGSNILPKLLENDDSHAIDTMVALI | |
| | RSVLQMRNSNAATGEDYINSPVRDLNGVCFDSRFQNPEWPMDADANGAYHIALKGQLL | |
| | LNHLKESKDLKLQNGISNQDWLAYIQELRN | |
| 331 | MPKPAVESEFSKVLKKHFPGERFRSSYMKRGGKILAAQGEEAVVAYLQGKSEEEPPNE | dCasPhi-2 (aa) |
| | QPPAKCHVVTKSRDFAEWPIMKASEAIQRYIYALSTTERAACKPGKSSESHAAWFAAT | |
| | GVSNHGYSHVQGLNLIFDHTLGRYDGVLKKVQLRNEKARARLESINASRADEGLPEIK | |
| | AEEEEVATNETGHLLQPPGINPSFYVYQTISPQAYRPRDEIVLPPEYAGYVRDPNAPI | |
| | PLGVVRNRCDIQKGCPGYIPEWQREAGTAISPKTGKAVTVPGLSPKKNKRMRRYWRSE | |
| | KEKAQDALLVTVRIGTDWVVIDVRGLLRNARWRTIAPKDISLNALLDLFTGDPVIDVR | |
| | RNIVTFTYTLDACGTYARKWTLKGKQTKATLDKLTATQTVALVAIALGQTNPISAGIS | |
| | RVTQENGALQCEPLDRFTLPDDLLKDISAYRIAWDRNEEELRARSVEALPEAQQAEVR | |
| | ALDGVSKETARTQLCADFGLDPKRLPWDKMSSNTTFISEALLSNSVSRDQVFFTPAPK | |
| | KGAKKKAPVEVMRKDRTWARAYKPRLSVEAQKLKNEALWALKRTSPEYLKLSRRKEEL | |
| | CRRSINYVIEKTRRRTQCQIVIPVIEDLNVRFFHGSGKRLPGWDNFFTAKKENRWFIQ | |
| | GLHKAFSDLRTHRSFYVFEVRPERTSITCPKCGHCEVGNRDGEAFQCLSCGKTCNADL | |
| | DVATHNLTQVALTGKTMPKREEPRDAQGTAPARKTKKASKSKAPPAEREDQTPAQEPS | |
| | QTSGSGPKKKRKVEDPKKKRKV | |
| 332 | PKPAVESEFSKVLKKHFPGERFRSSYMKRGGKILAAQGEEAVVAYLQGKSEEEPPNFQ | dCasPhi-2 (aa) |
| | PPAKCHVVTKSRDFAEWPIMKASEAIQRYIYALSTTERAACKPGKSSESHAAWFAATG | |
| | VSNHGYSHVQGLNLIFDHTLGRYDGVLKKVQLRNEKARARLESINASRADEGLPEIKA | |
| | EEEEVATNETGHLLQPPGINPSFYVYQTISPQAYRPRDEIVLPPEYAGYVRDPNAPIP | |
| | LGVVRNRCDIQKGCPGYIPEWQREAGTAISPKTGKAVTVPGLSPKKNKRMRRYWRSEK | |
| | EKAQDALLVTVRIGTDWVVIDVRGLLRNARWRTIAPKDISLNALLDLFTGDPVIDVRR | |
| | NIVTFTYTLDACGTYARKWTLKGKQTKATLDKLTATQTVALVAIALGQTNPISAGISR | |
| | VTQENGALQCEPLDRFTLPDDLLKDISAYRIAWDRNEEELRARSVEALPEAQQAEVRA | |
| | LDGVSKETARTQLCADFGLDPKRLPWDKMSSNTTFISEALLSNSVSRDQVFFTPAPKK | |
| | GAKKKAPVEVMRKDRTWARAYKPRLSVEAQKLKNEALWALKRTSPEYLKLSRRKEELC | |
| | RRSINYVIEKTRRRTQCQIVIPVIEDLNVRFFHGSGKRLPGWDNFFTAKKENRWFIQG | |
| | LHKAFSDLRTHRSFYVFEVRPERTSITCPKCGHCEVGNRDGEAFQCLSCGKTCNADLD | |
| | VATHNLTQVALTGKTMPKREEPRDAQGTAPARKTKKASKSKAPPAEREDQTPAQEPSQ | |
| | TSGSGPKKKRKVEDPKKKRKV | |
| 333 | MPKPAVESEFSKVLKKHFPGERFRSSYMKRGGKILAAQGEEAVVAYLQGKSEEEPPNF | WT CasPhi-2 (aa) |
| | QPPAKCHVVTKSRDFAEWPIMKASEAIQRYIYALSTTERAACKPGKSSESHAAWFAAT | |
| | GVSNHGYSHVQGLNLIFDHTLGRYDGVLKKVQLRNEKARARLESINASRADEGLPEIK | |
| | AEEEEVATNETGHLLQPPGINPSFYVYQTISPQAYRPRDEIVLPPEYAGYVRDPNAPI | |
| | PLGVVRNRCDIQKGCPGYIPEWQREAGTAISPKTGKAVTVPGLSPKKNKRMRRYWRSE | |
| | KEKAQDALLVTVRIGTDWVVIDVRGLLRNARWRTIAPKDISLNALLDLFTGDPVIDVR | |
| | RNIVTFTYTLDACGTYARKWTLKGKQTKATLDKLTATQTVALVAIDLGQTNPISAGIS | |
| | RVTQENGALQCEPLDRFTLPDDLLKDISAYRIAWDRNEEELRARSVEALPEAQQAEVR | |
| | ALDGVSKETARTQLCADFGLDPKRLPWDKMSSNTTFISEALLSNSVSRDQVFFTPAPK | |
| | KGAKKKAPVEVMRKDRTWARAYKPRLSVEAQKLKNEALWALKRTSPEYLKLSRRKEEL | |
| | CRRSINYVIEKTRRRTQCQIVIPVIEDLNVRFFHGSGKRLPGWDNFFTAKKENRWFIQ | |
| | GLHKAFSDLRTHRSFYVFEVRPERTSITCPKCGHCEVGNRDGEAFQCLSCGKTCNADL | |
| | DVATHNLTQVALTGKTMPKREEPRDAQGTAPARKTKKASKSKAPPAEREDQTPAQEPS | |
| | QTS | |
| 334 | PKPAVESEFSKVLKKHFPGERFRSSYMKRGGKILAAQGEEAVVAYLQGKSEEEPPNFQ | WT CasPhi-2 (aa) |
| | PPAKCHVVTKSRDFAEWPIMKASEAIQRYIYALSTTERAACKPGKSSESHAAWFAATG | |
| | VSNHGYSHVQGLNLIFDHTLGRYDGVLKKVQLRNEKARARLESINASRADEGLPEIKA | |
| | EEEEVATNETGHLLQPPGINPSFYVYQTISPQAYRPRDEIVLPPEYAGYVRDPNAPIP | |
| | LGVVRNRCDIQKGCPGYIPEWQREAGTAISPKTGKAVTVPGLSPKKNKRMRRYWRSEK | |
| | EKAQDALLVTVRIGTDWVVIDVRGLLRNARWRTIAPKDISLNALLDLFTGDPVIDVRR | |
| | NIVTFTYTLDACGTYARKWTLKGKQTKATLDKLTATQTVALVAIDLGQTNPISAGISR | |
| | VTQENGALQCEPLDRFTLPDDLLKDISAYRIAWDRNEEELRARSVEALPEAQQAEVRA | |
| | LDGVSKETARTQLCADFGLDPKRLPWDKMSSNTTFISEALLSNSVSRDQVFFTPAPKK | |
| | GAKKKAPVEVMRKDRTWARAYKPRLSVEAQKLKNEALWALKRTSPEYLKLSRRKEELC | |
| | RRSINYVIEKTRRRTQCQIVIPVIEDLNVRFFHGSGKRLPGWDNFFTAKKENRWFIQG | |
| | LHKAFSDLRTHRSFYVFEVRPERTSITCPKCGHCEVGNRDGEAFQCLSCGKTCNADLD | |
| | VATHNLTQVALTGKTMPKREEPRDAQGTAPARKTKKASKSKAPPAEREDQTPAQEPSQ | |
| | TS | |
| 335 | MAKNTITKTLKLRIVRPYNSAEVEKIVADEKNNREKIALEKNKDKVKEACSKHLKVAA | WT UnlCas12fl |
| | YCTTQVERNACLFCKARKLDDKFYQKLRGQFPDAVFWQEISEIFRQLQKQAAEIYNQS | (aa) |
| | LIELYYEIFIKGKGIANASSVEHYLSDVCYTRAAELFKNAAIASGLRSKIKSNERLKE | |
| | LKNMKSGLPTTKSDNFPIPLVKQKGGQYTGFEISNHNSDFIIKIPFGRWQVKKEIDKY | |
| | RPWEKFDFEQVQKSPKPISLLLSTQRRKRNKGWSKDEGTEAEIKKVMNGDYQTSYIEV | |
| | KRGSKIGEKSAWMLNLSIDVPKIDKGVDPSIIGGIDVGVKSPLVCAINNAFSRYSISD | |
| | NDLFHFNKKMFARRRILLKKNRHKRAGHGAKNKLKPITILTEKSERFRKKLIERWACE | |
| | IADFFIKNKVGTVQMENLESMKRKEDSYFNIRLRGFWPYAEMQNKIEFKLKQYGIEIR | |
| | KVAPNNTSKTCSKCGHLNNYFNFEYRKKNKFPHFKCEKCNFKENADYNAALNISNPKL | |
| | KSTKEEP | |
| 336 | AKNTITKTLKLRIVRPYNSAEVEKIVADEKNNREKIALEKNKDKVKEACSKHLKVAAY | WT Un1Cas12f1 |
| | CTTQVERNACLFCKARKLDDKFYQKLRGQFPDAVFWQEISEIFRQLQKQAAEIYNQSL | (aa) |
| | IELYYEIFIKGKGIANASSVEHYLSDVCYTRAAELFKNAAIASGLRSKIKSNFRLKEL | |
| | KNMKSGLPTTKSDNFPIPLVKQKGGQYTGFEISNHNSDFIIKIPFGRWQVKKEIDKYR | |
| | PWEKFDFEQVQKSPKPISLLLSTQRRKRNKGWSKDEGTEAEIKKVMNGDYQTSYIEVK | |
| | RGSKIGEKSAWMLNLSIDVPKIDKGVDPSIIGGIDVGVKSPLVCAINNAFSRYSISDN | |
| | DLFHFNKKMFARRRILLKKNRHKRAGHGAKNKLKPITILTEKSERFRKKLIERWACEI | |
| | ADFFIKNKVGTVQMENLESMKRKEDSYFNIRLRGFWPYAEMQNKIEFKLKQYGIEIRK | |
| | VAPNNTSKTCSKCGHLNNYFNFEYRKKNKFPHFKCEKCNFKENADYNAALNISNPKLK | |
| | STKEEP | |
| 337 | MAKNTITKTLKLRIVRPYNSAEVEKIVADEKNNREKIALEKNKDKVKEACSKHLKVAA | dUn1Cas12fl (aa) |
| | YCTTQVERNACLFCKARKLDDKFYQKLRGQFPDAVFWQEISEIFRQLQKQAAEIYNQS | |
| | LIELYYEIFIKGKGIANASSVEHYLSDVCYTRAAELFKNAAIASGLRSKIKSNFRLKE | |
| | LKNMKSGLPTTKSDNFPIPLVKQKGGQYTGFEISNHNSDFIIKIPFGRWQVKKEIDKY | |
| | RPWEKFDFEQVQKSPKPISLLLSTQRRKRNKGWSKDEGTEAEIKKVMNGDYQTSYIEV | |
| | KRGSKIGEKSAWMLNLSIDVPKIDKGVDPSIIGGIAVGVKSPLVCAINNAFSRYSISD | |
| | NDLFHFNKKMFARRRILLKKNRHKRAGHGAKNKLKPITILTEKSERFRKKLIERWACE | |
| | IADFFIKNKVGTVQMENLESMKRKEDSYFNIRLRGFWPYAEMQNKIEFKLKQYGIEIR | |
| | KVAPNNTSKTCSKCGHLNNYFNFEYRKKNKFPHFKCEKCNFKENAAYNAALNISNPKL | |
| | KSTKEEP | |
| 338 | AKNTITKTLKLRIVRPYNSAEVEKIVADEKNNREKIALEKNKDKVKEACSKHLKVAAY | dUnlCas12fl (aa) |
| | CTTQVERNACLFCKARKLDDKFYQKLRGQFPDAVEWQEISEIFRQLQKQAAEIYNQSL | |
| | IELYYEIFIKGKGIANASSVEHYLSDVCYTRAAELFKNAAIASGLRSKIKSNERLKEL | |
| | KNMKSGLPTTKSDNFPIPLVKQKGGQYTGFEISNHNSDFIIKIPFGRWQVKKEIDKYR | |
| | PWEKFDFEQVQKSPKPISLLLSTQRRKRNKGWSKDEGTEAEIKKVMNGDYQTSYIEVK | |
| | RGSKIGEKSAWMLNLSIDVPKIDKGVDPSIIGGIAVGVKSPLVCAINNAFSRYSISDN | |
| | DLFHENKKMFARRRILLKKNRHKRAGHGAKNKLKPITILTEKSERFRKKLIERWACEI | |
| | ADFFIKNKVGTVQMENLESMKRKEDSYFNIRLRGFWPYAEMQNKIEFKLKQYGIEIRK | |
| | VAPNNTSKTCSKCGHLNNYFNFEYRKKNKFPHFKCEKCNFKENAAYNAALNISNPKLK | |
| | STKEEP | |
| 339 | MARILAFAIGISSIGWAFSENDELKDCGVRIFTKVENPKTGESLALPRRLARSARKRL | dCjCas9 (aa) |
| | ARRKARLNHLKHLIANEFKLNYEDYQSFDESLAKAYKGSLISPYELRFRALNELLSKQ | |
| | DFARVILHIAKRRGYDDIKNSDDKEKGAILKAIKQNEEKLANYQSVGEYLYKEYFQKF | |
| | KENSKEFTNVRNKKESYERCIAQSFLKDELKLIFKKQREFGFSFSKKFEEEVLSVAFY | |
| | KRALKDFSHLVGNCSFFTDEKRAPKNSPLAFMFVALTRIINLLNNLKNTEGILYTKDD | |
| | LNALLNEVLKNGTLTYKQTKKLLGLSDDYEFKGEKGTYFIEFKKYKEFIKALGEHNLS | |
| | QDDLNEIAKDITLIKDEIKLKKALAKYDLNQNQIDSLSKLEFKDHLNISFKALKLVTP | |
| | LMLEGKKYDEACNELNLKVAINEDKKDFLPAFNETYYKDEVTNPVVLRAIKEYRKVLN | |
| | ALLKKYGKVHKINIELAREVGKNHSQRAKIEKEQNENYKAKKDAELECEKLGLKINSK | |
| | NILKLRLFKEQKEFCAYSGEKIKISDLQDEKMLEIDAIYPYSRSFDDSYMNKVLVFTK | |
| | QNQEKLNQTPFEAFGNDSAKWQKIEVLAKNLPTKKQKRILDKNYKDKEQKNFKDRNLN | |
| | DTRYIARLVLNYTKDYLDFLPLSDDENTKLNDTQKGSKVHVEAKSGMLTSALRHTWGF | |
| | SAKDRNNHLHHAIDAVIIAYANNSIVKAFSDFKKEQESNSAELYAKKISELDYKNKRK | |
| | FFEPFSGFRQKVLDKIDEIFVSKPERKKPSGALHEETFRKEEEFYQSYGGKEGVLKAL | |
| | ELGKIRKVNGKIVKNGDMFRVDIFKHKKTNKFYAVPIYTMDFALKVLPNKAVARSKKG | |
| | EIKDWILMDENYEFCFSLYKDSLILIQTKDMQEPEFVYYNAFTSSTVSLIVSKHDNKF | |
| | ETLSKNQKILFKNANEKEVIAKSIGIQNLKVFEKYIVSALGEVTKAEFRQREDFKK | |
| 340 | ARILAFAIGISSIGWAFSENDELKDCGVRIFTKVENPKTGESLALPRRLARSARKRLA | dCjCas9 (aa) |
| | RRKARLNHLKHLIANEFKLNYEDYQSFDESLAKAYKGSLISPYELRFRALNELLSKQD | |
| | FARVILHIAKRRGYDDIKNSDDKEKGAILKAIKQNEEKLANYQSVGEYLYKEYFQKFK | |
| | ENSKEFTNVRNKKESYERCIAQSFLKDELKLIFKKQREFGFSFSKKFEEEVLSVAFYK | |
| | RALKDFSHLVGNCSFFTDEKRAPKNSPLAFMFVALTRIINLLNNLKNTEGILYTKDDL | |
| | NALLNEVLKNGTLTYKQTKKLLGLSDDYEFKGEKGTYFIEFKKYKEFIKALGEHNLSQ | |
| | DDLNEIAKDITLIKDEIKLKKALAKYDLNQNQIDSLSKLEFKDHLNISFKALKLVTPL | |
| | MLEGKKYDEACNELNLKVAINEDKKDFLPAFNETYYKDEVTNPVVLRAIKEYRKVLNA | |
| | LLKKYGKVHKINIELAREVGKNHSQRAKIEKEQNENYKAKKDAELECEKLGLKINSKN | |
| | ILKLRLFKEQKEFCAYSGEKIKISDLQDEKMLEIDAIYPYSRSFDDSYMNKVLVFTKQ | |
| | NQEKLNQTPFEAFGNDSAKWQKIEVLAKNLPTKKQKRILDKNYKDKEQKNFKDRNLND | |
| | TRYIARLVLNYTKDYLDFLPLSDDENTKLNDTQKGSKVHVEAKSGMLTSALRHTWGFS | |
| | AKDRNNHLHHAIDAVIIAYANNSIVKAFSDFKKEQESNSAELYAKKISELDYKNKRKF | |
| | FEPFSGFRQKVLDKIDEIFVSKPERKKPSGALHEETFRKEEEFYQSYGGKEGVLKALE | |
| | LGKIRKVNGKIVKNGDMFRVDIFKHKKTNKFYAVPIYTMDFALKVLPNKAVARSKKGE | |
| | IKDWILMDENYEFCFSLYKDSLILIQTKDMQEPEFVYYNAFTSSTVSLIVSKHDNKFE | |
| | TLSKNQKILFKNANEKEVIAKSIGIQNLKVFEKYIVSALGEVTKAEFRQREDFKK | |
| 341 | MSSGSGHMARILAFDIGISSIGWAFSENDELKDCGVRIFTKVENPKTGESLALPRRLA | WT CjCas9 (aa) |
| | RSARKRLARRKARLNHLKHLIANEFKLNYEDYQSFDESLAKAYKGSLISPYELRFRAL | |
| | NELLSKQDFARVILHIAKRRGYDDIKNSDDKEKGAILKAIKQNEEKLANYQSVGEYLY | |
| | KEYFQKFKENSKEFTNVRNKKESYERCIAQSFLKDELKLIFKKQREFGFSFSKKFEEE | |
| | VLSVAFYKRALKDFSHLVGNCSFFTDEKRAPKNSPLAFMFVALTRIINLLNNLKNTEG | |
| | ILYTKDDLNALLNEVLKNGTLTYKQTKKLLGLSDDYEFKGEKGTYFIEFKKYKEFIKA | |
| | LGEHNLSQDDLNEIAKDITLIKDEIKLKKALAKYDLNQNQIDSLSKLEFKDHLNISFK | |
| | ALKLVTPLMLEGKKYDEACNELNLKVAINEDKKDFLPAFNETYYKDEVTNPVVLRAIK | |
| | EYRKVLNALLKKYGKVHKINIELGGGSGGYIARLVLNYTKDYLDFLPLSDDENTKLND | |
| | TQKGSKVHVEAKSGMLTSALRHTWGFSAKDRNNHLHHAIDAVIIAYANNSIVKAFSDF | |
| | KKEQESNSAELYAKKISELDYKNKRKFFEPFSGFRQKVLDKIDEIFVSKPERKKPSGA | |
| | LHEETFRKEEEFYQSYGGKEGVLKALELGKIRKVNGKIVKNGDMFRVDIFKHKKTNKF | |
| | YAVPIYTMDFALKVLPNKAVARSKKGEIKDWILMDENYEFCFSLYKDSLILIQTKDMQ | |
| | EPEFVYYNAFTSSTVSLIVSKHDNKFETLSKNQKILFKNANEKEVIAKSIGIQNLKVE | |
| | EKYIVSALGEVTKAEFRQREDFKK | |
| 342 | SSGSGHMARILAFDIGISSIGWAFSENDELKDCGVRIFTKVENPKTGESLALPRRLAR | WT CjCas9 (aa) |
| | SARKRLARRKARLNHLKHLIANEFKLNYEDYQSFDESLAKAYKGSLISPYELRFRALN | |
| | ELLSKQDFARVILHIAKRRGYDDIKNSDDKEKGAILKAIKQNEEKLANYQSVGEYLYK | |
| | EYFQKFKENSKEFTNVRNKKESYERCIAQSFLKDELKLIFKKQREFGFSFSKKFEEEV | |
| | LSVAFYKRALKDFSHLVGNCSFFTDEKRAPKNSPLAFMFVALTRIINLLNNLKNTEGI | |
| | LYTKDDLNALLNEVLKNGTLTYKQTKKLLGLSDDYEFKGEKGTYFIEFKKYKEFIKAL | |
| | GEHNLSQDDLNEIAKDITLIKDEIKLKKALAKYDLNQNQIDSLSKLEFKDHLNISEKA | |
| | LKLVTPLMLEGKKYDEACNELNLKVAINEDKKDELPAFNETYYKDEVTNPVVLRAIKE | |
| | YRKVLNALLKKYGKVHKINIELGGGSGGYIARLVLNYTKDYLDFLPLSDDENTKLNDT | |
| | QKGSKVHVEAKSGMLTSALRHTWGFSAKDRNNHLHHAIDAVIIAYANNSIVKAFSDEK | |
| | KEQESNSAELYAKKISELDYKNKRKFFEPFSGFRQKVLDKIDEIFVSKPERKKPSGAL | |
| | HEETFRKEEEFYQSYGGKEGVLKALELGKIRKVNGKIVKNGDMFRVDIFKHKKTNKFY | |
| | AVPIYTMDFALKVLPNKAVARSKKGEIKDWILMDENYEFCFSLYKDSLILIQTKDMQE | |
| | PEFVYYNAFTSSTVSLIVSKHDNKFETLSKNQKILFKNANEKEVIAKSIGIQNLKVFE | |
| | KYIVSALGEVTKAEFRQREDFKK | |
| 343 | MSSTSGKNPTNPIVRTISCNLSAKEDVLRKVWEEMSQKNTPLIVQLLKSVSEQPEFEA | WT Cas12k (aa) |
| | NKENGTITKKEITELRRDITEDSDLKKQSGRLRSSADSLVTEVYSSWIRLYQVRKNKK | |
| | EGKEYFLNNILKSDVELVEQSNCDLQTIRCKAKEILSQVEEFIEQVNNKPKINKTTSA | |
| | KKKINKSNKNNKAIEEILNRFFIGNIDKTLTNTLYEIHRKSPDILTQCAVAYLIKNDN | |
| | KVSEAEENLTQLNKRSSAKEIEIKRLETQIQNTRLPNGRDITGEKYSQAFEKLVNQVP | |
| | QDNEEFAEWIAILLKKVSSLPYPILYSSGDLSWYKDEKGNIFVYENGWAEYHFQICCD | |
| | KRQLRFFERFLKYYKALKASEKGEEKLSGSLVTLRSAHLLWRQGKGKGEPWKVNKLAL | |
| | HCTYDARLWTAEGTEEVRQEKTDKAQAEVNQAESNENIDSKQQKKLTKNKSSLSRLKN | |
| | SFARPSKPLYRGQSNIIVGISFHPVELATLVVVDINTKEILICKTVKELLGDAFPLLS | |
| | RRRRQQVHFRKEREKAQKKDSPCDLGESKLGEYVDRLLAKRIVEVAKEYQASCIVLPG | |
| | LKGIREIRTSVIQAKAETKFPGDINAQELYVKEYNRQIHNWSYSRLQESIKSRAAELK | |
| | ISIKFGKQPSHSTLQEQAINLALSA | |
| 344 | SSTSGKNPTNPIVRTISCNLSAKEDVLRKVWEEMSQKNTPLIVQLLKSVSEQPEFEAN | WT Cas12k (aa) |
| | KENGTITKKEITELRRDITEDSDLKKQSGRLRSSADSLVTEVYSSWIRLYQVRKNKKE | |
| | GKEYFLNNILKSDVELVEQSNCDLQTIRCKAKEILSQVEEFIEQVNNKPKINKTTSAK | |
| | KKINKSNKNNKAIEEILNRFFIGNIDKTLTNTLYEIHRKSPDILTQCAVAYLIKNDNK | |
| | VSEAEENLTQLNKRSSAKEIEIKRLETQIQNTRLPNGRDITGEKYSQAFEKLVNQVPQ | |
| | DNEEFAEWIAILLKKVSSLPYPILYSSGDLSWYKDEKGNIFVYFNGWAEYHFQICCDK | |
| | RQLRFFERFLKYYKALKASEKGEEKLSGSLVTLRSAHLLWRQGKGKGEPWKVNKLALH | |
| | CTYDARLWTAEGTEEVRQEKTDKAQAEVNQAESNENIDSKQQKKLTKNKSSLSRLKNS | |
| | FARPSKPLYRGQSNIIVGISFHPVELATLVVVDINTKEILICKTVKELLGDAFPLLSR | |
| | RRRQQVHFRKEREKAQKKDSPCDLGESKLGEYVDRLLAKRIVEVAKEYQASCIVLPGL | |
| | KGIREIRTSVIQAKAETKFPGDINAQELYVKEYNRQIHNWSYSRLQESIKSRAAELKI | |
| | SIKFGKQPSHSTLQEQAINLALSA | |
| 345 | MGPKKKRKVGSGSAKNTITKTLKLRIVRPYNSAEVEKIVADEKNNREKIALEKNKDKV | CasMini |
| | KEACSKHLKVAAYCTTQVERNACLFCKARKLDDKFYQKLRGQFPDAVFWQEISEIFRQ | |
| | LQKQAAEIYNQSLIELYYEIFIKGKGIANASSVEHYLSRVCYRRAAELFKNAAIASGL | |
| | RSKIKSNFRLKELKNMKSGLPTTKSDNFPIPLVKQKGGQYTGFEISNHNSDFIIKIPF | |
| | GRWQVKKEIDKYRPWEKFDFEQVQKSPKPISLLLSTQRRKRNKGWSKDEGTEAEIKKV | |
| | MNGDYQTSYIEVKRGSKICEKSAWMLNLSIDVPKIDKGVDPSIIGGIAVGVRSPLVCA | |
| | INNAFSRYSISDNDLFHFNKKMFARRRILLKKNRHKRAGHGAKNKLKPITILTEKSER | |
| | FRKKLIERWACEIADFFIKNKVGTVQMENLESMKRKEDSYFNIRLRGFWPYAEMQNKI | |
| | EFKLKQYGIEIRKVAPNNTSKTCSKCGHLNNYFNFEYRKKNKFPHFKCEKCNFKENAA | |
| | YNAALNISNPKLKSTKERPAKRPAATKKAGQAKKKK | |
| 346 | GPKKKRKVGSGSAKNTITKTLKLRIVRPYNSAEVEKIVADEKNNREKIALEKNKDKVK | CasMini |
| | EACSKHLKVAAYCTTQVERNACLFCKARKLDDKFYQKLRGQFPDAVFWQEISEIFRQL | |
| | QKQAAEIYNQSLIELYYEIFIKGKGIANASSVEHYLSRVCYRRAAELFKNAAIASGLR | |
| | SKIKSNFRLKELKNMKSGLPTTKSDNFPIPLVKQKGGQYTGFEISNHNSDFIIKIPFG | |
| | RWQVKKEIDKYRPWEKFDFEQVQKSPKPISLLLSTQRRKRNKGWSKDEGTEAEIKKVM | |
| | NGDYQTSYIEVKRGSKICEKSAWMLNLSIDVPKIDKGVDPSIIGGIAVGVRSPLVCAI | |
| | NNAFSRYSISDNDLFHENKKMFARRRILLKKNRHKRAGHGAKNKLKPITILTEKSERF | |
| | RKKLIERWACEIADFFIKNKVGTVQMENLESMKRKEDSYFNIRLRGFWPYAEMQNKIE | |
| | FKLKQYGIEIRKVAPNNTSKTCSKCGHLNNYENFEYRKKNKFPHFKCEKCNFKENAAY | |
| | NAALNISNPKLKSTKERPAKRPAATKKAGQAKKKK | |
| 347 | TGGAAGTAAGGCAAAAAGATTGC | Cas12a gRNA 1 |
| | | spacer |
| 348 | AGGCAAAAAGATTGCTTTGAATA | Cas12a gRNA 2 |
| | | spacer |
| 349 | AGTGGTTCTCAACCAGGGGCAAT | Cas12a gRNA 3 |
| | | spacer |
| 350 | CAATTTTACTCCTTAGGGGACCT | Cas12a gRNA 4 |
| | | spacer |
| 351 | TTTTCACAATGTCTGGAGACATT | Cas12a gRNA 5 |
| | | spacer |
| 352 | TGGGAAATGCATCATTAGGTGAT | Cas12a gRNA 6 |
| | | spacer |
| 353 | AATTGTACTACAGTAGTTAAGTA | Cas12a gRNA 7 |
| | | spacer |
| 354 | AGTATGGTAACATGCTGTACAGG | Cas12a gRNA 8 |
| | | spacer |
| 355 | TATAGTAGGCTAGACCATCTAGC | Cas12a gRNA 9 |
| | | spacer |
| 356 | TCACAAAATCACCTAATGATGCA | Cas12a gRNA 10 |
| | | spacer |
| 357 | TTGTCCCATGCATTGTAAGGTGG | Cas12a gRNA 11 |
| | | spacer |
| 358 | GAGAACCACTATTCTGGTCTAAC | Cas12a gRNA 12 |
| | | spacer |
| 359 | AATTAAATATTCAAAGCAATCTT | Cas12a gRNA 13 |
| | | spacer |
| 360 | ATCTTTTTGCCTTACTTCCAAGT | Cas12a gRNA 14 |
| | | spacer |
| 361 | CTTACTTCCAAGTTTTAGATAAT | Cas12a gRNA 15 |
| | | spacer |
| 362 | GCTCACGCCTGTCGTCTCAGCAC | Cas12a gRNA 16 |
| | | spacer |
| 363 | GTGAGGCCCAGGCGGGCAGATCG | Cas12a gRNA 17 |
| | | spacer |
| 364 | CAGCAGCTCCCAAGTTCCTCCTG | Cas12a gRNA 18 |
| | | spacer |
| 365 | CCCAAGTTCCTCCTGTTTAGAAT | Cas12a gRNA 19 |
| | | spacer |
| 366 | TAATATTTTCAAGGCTGGATTTT | Cas12a gRNA 20 |
| | | spacer |
| 367 | CTGGATTTTTTTGAACGAAATGC | Cas12a gRNA 21 |
| | | spacer |
| 368 | TAGCTATTCTGCAGCTCTGAAAG | Cas12a gRNA 22 |
| | | spacer |
| 369 | TTTCACCTCGTTCCAGGAAAGCA | Cas12a gRNA 23 |
| | | spacer |
| 370 | TCACTGCAAGCTCTGCCTCCCGG | Cas12a gRNA 24 |
| | | spacer |
| 371 | CCGCCACCATGCCTGGCTAATTT | Cas12a gRNA 25 |
| | | spacer |
| 372 | ACACCCAGCTAGTTTTTGTATTT | Cas12a gRNA 26 |
| | | spacer |
| 373 | TTGTATTTTTTGTAGAAAGGGGG | Cas12a gRNA 27 |
| | | spacer |
| 374 | TCCCCTAAGGAGTAAAATTG | gRNA AD spacer |
| 375 | TCTAACTTTGAATTAAATAT | gRNA AE spacer |
| 376 | SGSETPGTSESATPES | Linker (aa) |
| 377 | EGQSDERALLDQLHTLLSNTDATGLEEIDRALGIPELVNQGQALEPKQDGGGG | Tripartite activator- |
| | SHEKFPSDLDLDMFNGSLECDMESIIRSELMDADGLDFNFDSLNGGGGSHEKFP | NCOA3, FOXO3, |
| | SDLDLDMFNGSLECDMESIIRSELMDADGLDFNFDSL | FOXO3 (aa) |
| 378 | AEEFVTLKDVGMDFTLGDWEQLGLEQGDTFWDTALDNCQDLFLLDPPRPNLT | ZNF473 (AA) |
| | SHPDGSEDLEPLAGGSPEATSPDVTETK | |
| 379 | ECSEAGLLQEGVQPEEFVAIADYAATDETQLSFLRGEKILILRQTTADWWWGE | ANM2 (AA) |
| | RAGCCGYIPANHVGKHVDEYDPEDTWQ | |
| 380 | PRPELPLPEGWEEARDFDGKVYYIDHTNRTTSWIDPRDRYTKPLTFADCISDEL | KIBRA (AA) |
| | PLGWEEAYDPQVGDYFIDHNTKTTQI | |
| 381 | EACNSHACQWDGGDCSLTMENPWANCSSPLPCWDYINNQCDELCNTVECLFD | NOTCH2 (NOTC2) |
| | NFECQGNSKTCKYDKYCADHFKDNHCDQ | (AA) |
| 382 | LVGSSLEGAVTPQTSAWLPPTSAEHDHSLSCVVTPQDGETSAQMIEENLNCLG | IKKA (AA) |
| | HLSTIIHEANEEQGNSMMNLDWSWLTE | |
| 383 | GSPSYGSPEDTDSFWNPNAFETDSDLPAGWMRVQDTSGTYYWHIPTGTTQWE | APBB1 (AA) |
| | PPGRASPSQGSSPQEESQLTWTGFAHGE | |
| 384 | GNREEQNLSDLLSPICEVANNIEQNAQENENESQVSTDESENSRSPGNKSDNIKP | SMN2 (AA) |
| | KSAPWNSFLPPPPPMPGPRLGPGKP | |
| 385 | LMDSLPGNFEITTSTGFLTDLTLDDILFADIDTSMYDFDPCTSSSGTASKMAPVS | SERTAD2 (AA) |
| | ADDLLKTLAPYSSQPVTPSQPFKMD | |
| 386 | FYIPVQIPGYQYVSPEGNCIEHVQPTSAFIQQPFIDEDPDKEKKIKELEMLLMSAE | MYBA (AA) |
| | NEVRRKRIPSQPGSFSSWSGSFLM | |
| 387 | MAEEFVTLKDVGMDFTLGDWEQLGLEQGDTFWDTALDNCQDLFLLDPPRPNL | ZNF473-Human, |
| | TSHPDGSEDLEPLAGGSPEATSPDVTETKNSPLMEDFFEEGFSQEIIEMLSKDGF | Full-length (AA) |
| | WNSNFGEACIEDTWLDSLLGDPESLLRSDIATNGESPTECKSHELKRGLSPVST | |
| | VSTGEDSMVHNVSEKTLTPAKSKEYRGEFFSYSDHSQQDSVQEGEKPYQCSEC | |
| | GKSFSGSYRLTQHWITHTREKPTVHQECEQGFDRNASLSVYPKTHTGYKFYVC | |
| | NEYGTTFSQSTYLWHQKTHTGEKPCKSQDSDHPPSHDTQPGEHQKTHTDSKSY | |
| | NCNECGKAFTRIFHLTRHQKIHTRKRYECSKCQATFNLRKHLIQHQKTHAAKT | |
| | TSECQECGKIFRHSSLLIEHQALHAGEEPYKCNERGKSFRHNSTLKIHQRVHSG | |
| | EKPYKCSECGKAFHRHTHLNEHRRIHTGYRPHKCQECVRSFSRPSHLMRHQAI | |
| | HTAEKPYSCAECKETFSDNNRLVQHQKMHTVKTPYECQECGERFICGSTLKCH | |
| | ESVHAREKQGFFVSGKILDQNPEQKEKCFKCNKCEKTFSCSKYLTQHERIHTRG | |
| | VKPFECDQCGKAFGQSTRLIHHQRIHSRVRLYKWGEQGKAISSASLIKLQSFHT | |
| | KEHPFKCNECGKTFSHSAHLSKHQLIHAGENPFKCSKCDRVFTQRNYLVQHER | |
| | THARKKPLVCNECGKTFRQSSCLSKHQRIHSGEKPYVCDYCGKAFGLSAELVR | |
| | HQRIHTGEKPYVCQECGKAFTQSSCLSIHRRVHTGEKPYRCGECGKAFAQKAN | |
| | LTQHQRIHTGEKPYSCNVCGKAFVLSAHLNQHLRVHTQETLYQCQRCQKAFR | |
| | CHSSLSRHQRVHNKQQYCL | |
| 388 | MATSGDCPRSESQGEEPAECSEAGLLQEGVQPEEFVAIADYAATDETQLSFLRG | ANM2-Human, |
| | EKILILRQTTADWWWGERAGCCGYIPANHVGKHVDEYDPEDTWQDEEYFGSY | Full-length (AA) |
| | GTLKLHLEMLADQPRTTKYHSVILQNKESLTDKVILDVGCGTGIISLFCAHYAR | |
| | PRAVYAVEASEMAQHTGQLVLQNGFADIITVYQQKVEDVVLPEKVDVLVSEW | |
| | MGTCLLFEFMIESILYARDAWLKEDGVIWPTMAALHLVPCSADKDYRSKVLF | |
| | WDNAYEFNLSALKSLAVKEFFSKPKYNHILKPEDCLSEPCTILQLDMRTVQISD | |
| | LETLRGELRFDIRKAGTLHGFTAWFSVHFQSLQEGQPPQVLSTGPFHPTTHWK | |
| | QTLFMMDDPVPVHTGDVVTGSVVLQRNPVWRRHMSVALSWAVTSRQDPTSQ | |
| | KVGEKVFPIWR | |
| 389 | MPRPELPLPEGWEEARDFDGKVYYIDHTNRTTSWIDPRDRYTKPLTFADCISDE | KIBRA-Human, |
| | LPLGWEEAYDPQVGDYFIDHNTKTTQIEDPRVQWRREQEHMLKDYLVVAQEA | Full-length (AA) |
| | LSAQKEIYQVKQQRLELAQQEYQQLHAVWEHKLGSQVSLVSGSSSSSKYDPEI | |
| | LKAEIATAKSRVNKLKREMVHLQHELQFKERGFQTLKKIDKKMSDAQGSYKL | |
| | DEAQAVLRETKAIKKAITCGEKEKQDLIKSLAMLKDGFRTDRGSHSDLWSSSSS | |
| | LESSSFPLPKQYLDVSSQTDISGSFGINSNNQLAEKVRLRLRYEEAKRRIANLKI | |
| | QLAKLDSEAWPGVLDSERDRLILINEKEELLKEMRFISPRKWTQGEVEQLEMA | |
| | RKRLEKDLQAARDTQSKALTERLKLNSKRNQLVRELEEATRQVATLHSQLKSL | |
| | SSSMQSLSSGSSPGSLTSSRGSLVASSLDSSTSASFTDLYYDPFEQLDSELQSKVE | |
| | FLLLEGATGFRPSGCITTIHEDEVAKTQKAEGGGRLQALRSLSGTPKSMTSLSPR | |
| | SSLSSPSPPCSPLMADPLLAGDAFLNSLEFEDPELSATLCELSLGNSAQERYRLE | |
| | EPGTEGKQLGQAVNTAQGCGLKVACVSAAVSDESVAGDSGVYEASVQRLGA | |
| | SEAAAFDSDESEAVGATRIQIALKYDEKNKQFAILIIQLSNLSALLQQQDQKVNI | |
| | RVAVLPCSESTTCLFRTRPLDASDTLVFNEVFWVSMSYPALHQKTLRVDVCTT | |
| | DRSHLEECLGGAQISLAEVCRSGERSTRWYNLLSYKYLKKQSRELKPVGVMAP | |
| | ASGPASTDAVSALLEQTAVELEKRQEGRSSTQTLEDSWRYEETSENEAVAEEE | |
| | EEEVEEEEGEEDVFTEKASPDMDGYPALKVDKETNTETPAPSPTVVRPKDRRV | |
| | GTPSQGPFLRGSTIIRSKTFSPGPQSQYVCRLNRSDSDSSTLSKKPPFVRNSLERR | |
| | SVRMKRPSSVKSLRSERLIRTSLDLELDLQATRTWHSQLTQEISVLKELKEQLE | |
| | QAKSHGEKELPQWLREDERFRLLLRMLEKRQMDRAEHKGELQTDKMMRAAA | |
| | KDVHRLRGQSCKEPPEVQSFREKMAFFTRPRMNIPALSADDV | |
| 390 | MPALRPALLWALLALWLCCAAPAHALQCRDGYEPCVNEGMCVTYHNGTGYC | NOTCH2 (NOTC2)- |
| | KCPEGFLGEYCQHRDPCEKNRCQNGGTCVAQAMLGKATCRCASGFTGEDCQ | Human, Full- |
| | YSTSHPCFVSRPCLNGGTCHMLSRDTYECTCQVGFTGKECQWTDACLSHPCA | length, alternative |
| | NGSTCTTVANQFSCKCLTGFTGQKCETDVNECDIPGHCQHGGTCLNLPGSYQC | isoform (AA) |
| | QCPQGFTGQYCDSLYVPCAPSPCVNGGTCRQTGDFTFECNCLPGFEGSTCERNI | |
| | DDCPNHRCQNGGVCVDGVNTYNCRCPPQWTGQFCTEDVDECLLQPNACQNG | |
| | GTCANRNGGYGCVCVNGWSGDDCSENIDDCAFASCTPGSTCIDRVASFSCMCP | |
| | EGKAGLLCHLDDACISNPCHKGALCDTNPLNGQYICTCPQGYKGADCTEDVD | |
| | ECAMANSNPCEHAGKCVNTDGAFHCECLKGYAGPRCEMDINECHSDPCQND | |
| | ATCLDKIGGFTCLCMPGFKGVHCELEINECQSNPCVNNGQCVDKVNRFQCLCP | |
| | PGFTGPVCQIDIDDCSSTPCLNGAKCIDHPNGYECQCATGFTGVLCEENIDNCD | |
| | PDPCHHGQCQDGIDSYTCICNPGYMGAICSDQIDECYSSPCLNDGRCIDLVNGY | |
| | QCNCQPGTSGVNCEINFDDCASNPCIHGICMDGINRYSCVCSPGFTGQRCNIDID | |
| | ECASNPCRKGATCINGVNGFRCICPEGPHHPSCYSQVNECLSNPCIHGNCTGGL | |
| | SGYKCLCDAGWVGINCEVDKNECLSNPCQNGGTCDNLVNGYRCTCKKGFKG | |
| | YNCQVNIDECASNPCLNQGTCFDDISGYTCHCVLPYTGKNCQTVLAPCSPNPC | |
| | ENAAVCKESPNFESYTCLCAPGWQGQRCTIDIDECISKPCMNHGLCHNTQGSY | |
| | MCECPPGFSGMDCEEDIDDCLANPCQNGGSCMDGVNTFSCLCLPGFTGDKCQ | |
| | TDMNECLSEPCKNGGTCSDYVNSYTCKCQAGFDGVHCENNINECTESSCFNGG | |
| | TCVDGINSFSCLCPVGFTGSFCLHEINECSSHPCLNEGTCVDGLGTYRCSCPLGY | |
| | TGKNCQTLVNLCSRSPCKNKGTCVQKKAESQCLCPSGWAGAYCDVPNVSCDI | |
| | AASRRGVLVEHLCQHSGVCINAGNTHYCQCPLGYTGSYCEEQLDECASNPCQ | |
| | HGATCSDFIGGYRCECVPGYQGVNCEYEVDECQNQPCQNGGTCIDLVNHFKC | |
| | SCPPGTRGLLCEENIDDCARGPHCLNGGQCMDRIGGYSCRCLPGFAGERCEGDI | |
| | NECLSNPCSSEGSLDCIQLTNDYLCVCRSAFTGRHCETFVDVCPQMPCLNGGTC | |
| | AVASNMPDGFICRCPPGFSGARCQSSCGQVKCRKGEQCVHTASGPRCFCPSPR | |
| | DCESGCASSPCQHGGSCHPQRQPPYYSCQCAPPFSGSRCELYTAPPSTPPATCLS | |
| | QYCADKARDGVCDEACNSHACQWDGGDCSLTMENPWANCSSPLPCWDYINN | |
| | QCDELCNTVECLFDNFECQGNSKTCKYDKYCADHFKDNHCDQGCNSEECGW | |
| | DGLDCAADQPENLAEGTLVIVVLMPPEQLLQDARSFLRALGTLLHTNLRIKRD | |
| | SQGELMVYPYYGEKSAAMKKQRMTRRSLPGEQEQEVAGSKVFLEIDNRQCVQ | |
| | DSDHCFKNTDAAAALLASHAIQGTLSYPLVSVVSESLTPERTQLLYLLAVAVVI | |
| | ILFIILLGVIMAKRKRKHGSLWLPEGFTLRRDASNHKRREPVGQDAVGLKNLSV | |
| | QVSEANLIGTGTSEHWVDDEGPQPKKVKAEDEALLSEEDDPIDRRPWTQQHLE | |
| | AADIRRTPSLALTPPQAEQEVDVLDVNVRGPDGCTPLMLASLRGGSSDLSDED | |
| | EDAEDSSANIITDLVYQGASLQAQTDRTGEMALHLAARYSRADAAKRLLDAG | |
| | ADANAQDNMGRCPLHAAVAADAQGVFQILIRNRVTDLDARMNDGTTPLILAA | |
| | RLAVEGMVAELINCQADVNAVDDHGKSALHWAAAVNNVEATLLLLKNGAN | |
| | RDMQDNKEETPLFLAAREGSYEAAKILLDHFANRDITDHMDRLPRDVARDRM | |
| | HHDIVRLLDEYNVTPSPPGTVLTSALSPVICGPNRSFLSLKHTPMGKKSRRPSAK | |
| | STMPTSLPNLAKEAKDAKGSRRKKSLSEKVQLSESSVTLSPVDSLESPHTYVSD | |
| | TTSSPMITSPGILQASPNPMLATAAPPAPVHAQHALSFSNLHEMQPLAHGASTV | |
| | LPSVSQLLSHHHIVSPGSGSAGSLSRLHPVPVPADWMNRMEVNETQYNEMFG | |
| | MVLAPAEGTHPGIAPQSRPPEGKHITTPREPLPPIVTFQLIPKGSIAQPAGAPQPQ | |
| | STCPPAVAGPLPTMYQIPEMARLPSVAFPTAMMPQQDGQVAQTILPAYHPFPA | |
| | SVGKYPTPPSQHSYASSNAAERTPSHSGHLQGEHPYLTPSPESPDQWSSSSPHSA | |
| | SDWSDVTTSPTPGGAGGGQRGPGTHMSEPPHNNMQVYA | |
| 391 | MERPPGLRPGAGGPWEMRERLGTGGFGNVCLYQHRELDLKIAIKSCRLELSTK | IKKA-Human, |
| | NRERWCHEIQIMKKLNHANVVKACDVPEELNILIHDVPLLAMEYCSGGDLRKL | Full-length (AA) |
| | LNKPENCCGLKESQILSLLSDIGSGIRYLHENKIIHRDLKPENIVLQDVGGKIIHKI | |
| | IDLGYAKDVDQGSLCTSFVGTLQYLAPELFENKPYTATVDYWSFGTMVFECIA | |
| | GYRPFLHHLQPFTWHEKIKKKDPKCIFACEEMSGEVRFSSHLPQPNSLCSLVVE | |
| | PMENWLQLMLNWDPQQRGGPVDLTLKQPRCFVLMDHILNLKIVHILNMTSAK | |
| | IISFLLPPDESLHSLQSRIERETGINTGSQELLSETGISLDPRKPASQCVLDGVRGC | |
| | DSYMVYLFDKSKTVYEGPFASRSLSDCVNYIVQDSKIQLPIIQLRKVWAEAVH | |
| | YVSGLKEDYSRLFQGQRAAMLSLLRYNANLTKMKNTLISASQQLKAKLEFFH | |
| | KSIQLDLERYSEQMTYGISSEKMLKAWKEMEEKAIHYAEVGVIGYLEDQIMSL | |
| | HAEIMELQKSPYGRRQGDLMESLEQRAIDLYKQLKHRPSDHSYSDSTEMVKIIV | |
| | HTVQSQDRVLKELFGHLSKLLGCKQKIIDLLPKVEVALSNIKEADNTVMFMQG | |
| | KRQKEIWHLLKIACTQSSARSLVGSSLEGAVTPQTSAWLPPTSAEHDHSLSCVV | |
| | TPQDGETSAQMIEENLNCLGHLSTIIHEANEEQGNSMMNLDWSWLTE | |
| 392 | MSVPSSLSQSAINANSHGGPALSLPLPLHAAHNQLLNAKLQATAVGPKDLRSA | APBB1-Human, |
| | MGEGGGPEPGPANAKWLKEGQNQLRRAATAHRDQNRNVTLTLAEEASQEPE | Full-length (AA) |
| | MAPLGPKGLIHLYSELELSAHNAANRGLRGPGLIISTQEQGPDEGEEKAAGEAE | |
| | EEEEDDDDEEEEEDLSSPPGLPEPLESVEAPPRPQALTDGPREHSKSASLLFGMR | |
| | NSAASDEDSSWATLSQGSPSYGSPEDTDSFWNPNAFETDSDLPAGWMRVQDT | |
| | SGTYYWHIPTGTTQWEPPGRASPSQGSSPQEESQLTWTGFAHGEGFEDGEFWK | |
| | DEPSDEAPMELGLKEPEEGTLTFPAQSLSPEPLPQEEEKLPPRNTNPGIKCFAVR | |
| | SLGWVEMTEEELAPGRSSVAVNNCIRQLSYHKNNLHDPMSGGWGEGKDLLLQ | |
| | LEDETLKLVEPQSQALLHAQPIISIRVWGVGRDSGRERDFAYVARDKLTQMLK | |
| | CHVFRCEAPAKNIATSLHEICSKIMAERRNARCLVNGLSLDHSKLVDVPFQVEF | |
| | PAPKNELVQKFQVYYLGNVPVAKPVGVDVINGALESVLSSSSREQWTPSHVSV | |
| | APATLTILHQQTEAVLGECRVRFLSFLAVGRDVHTFAFIMAAGPASFCCHMFW | |
| | CEPNAASLSEAVQAACMLRYQKCLDARSQASTSCLPAPPAESVARRVGWTVR | |
| | RGVQSLWGSLKPKRLGAHTP | |
| 393 | MAMSSGGSGGGVPEQEDSVLFRRGTGQSDDSDIWDDTALIKAYDKAVASFKH | SMN2-Human, |
| | ALKNGDICETSGKPKTTPKRKPAKKNKSQKKNTAASLQQWKVGDKCSAIWSE | Full-length (AA) |
| | DGCIYPATIASIDFKRETCVVVYTGYGNREEQNLSDLLSPICEVANNIEQNAQEN | |
| | ENESQVSTDESENSRSPGNKSDNIKPKSAPWNSFLPPPPPMPGPRLGPGKPGLKF | |
| | NGPPPPPPPPPPHLLSCWLPPFPSGPPIIPPPPPICPDSLDDADALGSMLISWYMSG | |
| | YHTGYYMGFRQNQKEGRCSHSLN | |
| 394 | MLGKGGKRKFDEHEDGLEGKIVSPCDGPSKVSYTLQRQTIFNISLMKLYNHRP | SERTAD2- |
| | LTEPSLQKTVLINNMLRRIQEELKQEGSLRPMFTPSSQPTTEPSDSYREAPPAFS | Human, Full-length |
| | HLASPSSHPCDLGSTTPLEACLTPASLLEDDDDTFCTSQAMQPTAPTKLSPPALL | (AA) |
| | PEKDSFSSALDEIEELCPTSTSTEAATAATDSVKGTSSEAGTQKLDGPQESRADD | |
| | SKLMDSLPGNFEITTSTGFLTDLTLDDILFADIDTSMYDFDPCTSSSGTASKMAP | |
| | VSADDLLKTLAPYSSQPVTPSQPFKMDLTELDHIMEVLVGS | |
| 395 | MAKRSRSEDEDDDLQYADHDYEVPQQKGLKKLWNRVKWTRDEDDKLKKLV | MYBA-Human, |
| | EQHGTDDWTLIASHLQNRSDFQCQHRWQKVLNPELIKGPWTKEEDQRVIELV | Full-length (AA) |
| | QKYGPKRWSLIAKHLKGRIGKQCRERWHNHLNPEVKKSSWTEEEDRIIYEAHK | |
| | RLGNRWAEIAKLLPGRTDNSIKNHWNSTMRRKVEQEGYLQDGIKSERSSSKLQ | |
| | HKPCAAMDHMQTQNQFYIPVQIPGYQYVSPEGNCIEHVQPTSAFIQQPFIDEDP | |
| | DKEKKIKELEMLLMSAENEVRRKRIPSQPGSFSSWSGSFLMDDNMSNTLNSLD | |
| | EHTSEFYSMDENQPVSAQQNSPTKFLAVEANAVLSSLQTIPEFAETLELIESDPV | |
| | AWSDVTSFDISDAAASPIKSTPVKLMRIQHNEGAMECQFNVSLVLEGKKNTCN | |
| | GGNSEAVPLTSPNIAKFSTPPAILRKKRKMRVGHSPGSELRDGSLNDGGNMAL | |
| | KHTPLKTLPFSPSQFFNTCPGNEQLNIENPSFTSTPICGQKALITTPLHKETTPKD | |
| | QKENVGFRTPTIRRSILGTTPRTPTPFKNALAAQEKKYGPLKIVSQPLAFLEEDIR | |
| | EVLKEETGTDLFLKEEDEPAYKSCKQENTASGKKVRKSLVLDNWEKEESGTQL | |
| | LTEDISDMQSENRFTTSLLMIPLLEIHDNRCNLIPEKQDINSTINKTYTLTKKKPNP | |
| | NTSKVVKLEKNLQSNCEWETVVYGKTEDQLIMTEQARRYLSTYTATSSTSRAL | |
| | IL | |

1. A fusion protein comprising:
two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2, wherein the transcriptional activation domain increases transcription of an endogenous locus when recruited to a target site at the endogenous locus.

2. The fusion protein of claim 1, further comprising a DNA-targeting domain or a component thereof.

3. A fusion protein comprising:
(1) a DNA-targeting domain or a component thereof, and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2, wherein the transcriptional activation domain increases transcription of an endogenous locus when recruited to a target site at the endogenous locus.

4. The fusion protein of claim 3, wherein the DNA-targeting domain comprises a Cas-gRNA combination comprising a Cas protein or a variant thereof, and at least one gRNA that binds to the target site at the endogenous locus, and the component thereof fused to the two or more transcriptional activation domains is the Cas protein or a variant thereof.

5. The fusion protein of claim 3, wherein the DNA-targeting domain comprises a zinc finger protein (ZFP), a transcription activator-like effector (TALE), a meganuclease, a homing endonuclease, or an I-SceI enzyme or a variant thereof that binds to the target site at the endogenous locus.

6. A fusion protein comprising:
(1) a Cas protein or a variant thereof, and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2, wherein the transcriptional activation domain increases transcription of an endogenous locus when recruited to a target site at the endogenous locus.

7. The fusion protein of claim 4 or 6, wherein the Cas protein or a variant thereof is capable of complexing with at least one gRNA.

8. A fusion protein comprising:
(1) a zinc finger protein (ZFP), a transcription activator-like effector (TALE), a meganuclease, a homing endonuclease, or an I-SceI enzyme or a variant thereof, and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2, wherein the transcriptional activation domain increases transcription of an endogenous locus when recruited to a target site at the endogenous locus.

9. The fusion protein of any of claims 4-8, wherein the variant thereof comprises a catalytically inactive variant.

10. The fusion protein of any of claims 4, 6, and 7, wherein the Cas protein or a variant thereof is a Cas9 or a variant thereof.

11. The fusion protein of any of claims 4, 6, 7, and 10, wherein the Cas protein or a variant thereof protein is a deactivated Cas9 (dCas9).

12. The fusion protein of any of claims 4, 6, 7, 10 and 11, wherein the Cas protein or a variant thereof is a Staphylococcus aureus Cas9 (SaCas9) or a variant thereof.

13. The fusion protein of any of claims 4, 6, 7, and 10-12, wherein the Cas protein or a variant thereof is a Staphylococcus aureus dCas9 (dSaCas9) that comprises at least one amino acid mutation selected from D10A and N580A, with reference to numbering of positions of SEQ ID NO:3.

14. The fusion protein of any of claims 4, 6, 7, and 10-13, wherein the Cas protein or a variant thereof comprises the sequence set forth in SEQ ID NO:2, and an amino acid sequence that has at least 90%, 91%, 92%, 93%, 94%, 95%, 96%, 97%, 98%, and 99% sequence identity thereto.

15. The fusion protein of any of claims 4, 6, 7, 10 and 11, wherein the Cas9 or variant thereof is a Streptococcus pyogenes Cas9 (SpCas9) or a variant thereof.

16. The fusion protein of any of claims 4, 6, 7, 10, 11, and 15, wherein the Cas protein or a variant thereof is a Streptococcus pyogenes dCas9 (dSpCas9) that comprises at least one amino acid mutation selected from D10A and H840A, with reference to numbering of positions of SEQ ID NO:7.

17. The fusion protein of any of claims 4, 6, 7, 10, 11, 15, and 16, wherein the Cas protein or a variant thereof comprises the sequence set forth in SEQ ID NO:6, and an amino acid sequence that has at least 90%, 91%, 92%, 93%, 94%, 95%, 96%, 97%, 98%, and 99% sequence identity thereto.

18. The fusion protein of any of claims 4, 6, 7, and 10-17, wherein the Cas protein or a variant thereof is a split variant Cas protein, wherein the split variant Cas protein comprises a first polypeptide comprising an N-terminal fragment of the variant Cas protein and an N-terminal Intein, and a second polypeptide comprising a C-terminal fragment of the variant Cas protein and a C-terminal Intein.

19. The fusion protein of claim 4, 6, 7, and 10-18, wherein when the first polypeptide and the second polypeptide of the split variant Cas protein are present in proximity or present in the same cell, the N-terminal Intein and C-terminal Intein self-excise and ligate the N-terminal fragment and the C-terminal fragment of the variant Cas protein to form a full-length variant Cas protein.

20. The fusion protein of claim 18 or 19, wherein the N-terminal Intein comprises an N-terminal Npu Intein, or the sequence set forth in SEQ ID NO:88, or an amino acid sequence that has at least 90%, 91%, 92%, 93%, 94%, 95%, 96%, 97%, 98%, or 99% sequence identity thereto, or a portion of any of the foregoing.

21. The fusion protein of any of claims 18-20, wherein the N-terminal fragment of the variant Cas protein comprises:
the N-terminal fragment of variant SpCas9 from the N-terminal end up to position 573 of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or
the sequence set forth in SEQ ID NO:86, or an amino acid sequence that has at least 90%, 91%, 92%, 93%, 94%, 95%, 96%, 97%, 98%, or 99% sequence identity thereto, or a portion of any of the foregoing.

22. The fusion protein of any of claims 18-21, wherein the C-terminal Intein comprises a C-terminal Npu Intein, or the sequence set forth in SEQ ID NO:92, or an amino acid sequence that has at least 90%, 91%, 92%, 93%, 94%, 95%, 96%, 97%, 98%, or 99% sequence identity thereto, or a portion of any of the foregoing.

23. The fusion protein of any of claims 18-22, wherein the C-terminal fragment of the variant Cas protein comprises:
the C-terminal fragment of variant SpCas9 from position 574 to the C-terminal end of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or
the sequence set forth in SEQ ID NO:94, or an amino acid sequence that has at least 90%, 91%, 92%, 93%, 94%, 95%, 96%, 97%, 98%, or 99% sequence identity thereto, or a portion of any of the foregoing.

24. The fusion protein of any of claims 4, 6, 7, or 10-23, wherein the Cas protein or a variant thereof is a Cpf1 or a variant thereof.

25. The fusion protein of any of claims 4, 6, and 7, wherein the Cas protein or a variant thereof is a variant Cpf1 that that is a deactivated Cpf1 (dCpf1).

26. The fusion protein of claim 24 or 25, wherein the variant comprises a catalytically inactive nuclease variant.

27. The fusion protein of any of claims 1-26, wherein the transcriptional activation domain of NCOA3 comprises:
(i) the sequence set forth in SEQ ID NO:40;
(ii) a contiguous portion of SEQ ID NO:40 of at least 20 amino acids;
(iii) the sequence set forth in SEQ ID NO:27;
(iv) a contiguous portion of SEQ ID NO:27 of at least 20 amino acids;
(v) an amino acid sequence that has at least 90%, 91%, 92%, 93%, 94%, 95%, 96%, 97%, 98%, or 99% sequence identity to any of the foregoing.

28. The fusion protein of any of claims 1-27, wherein the transcriptional activation domain of NCOA3 comprises the sequence set forth in SEQ ID NO:133 or an amino acid sequence that has at least 90%, 91%, 92%, 93%, 94%, 95%, 96%, 97%, 98%, or 99% sequence identity to SEQ ID NO:133.

29. The fusion protein of any of claims 1-28, wherein the transcriptional activation domain of NCOA3 comprises the sequence set forth in SEQ ID NO:133.

30. The fusion protein of any of claims 1-29, wherein the transcriptional activation domain of ENL comprises:
(i) the sequence set forth in SEQ ID NO:36;
(ii) a contiguous portion of SEQ ID NO:36 of at least 20 amino acids;
(iii) the sequence set forth in SEQ ID NO:23;
(iv) a contiguous portion of SEQ ID NO:23 of at least 20 amino acids;
(v) an amino acid sequence that has at least 90%, 91%, 92%, 93%, 94%, 95%, 96%, 97%, 98%, or 99% sequence identity to any of the foregoing.

31. The fusion protein of any of claims 1-30, wherein the transcriptional activation domain of ENL comprises the sequence set forth in SEQ ID NO:131 or an amino acid sequence that has at least 90%, 91%, 92%, 93%, 94%, 95%, 96%, 97%, 98%, or 99% sequence identity to any of the foregoing.

32. The fusion protein of any of claims 1-31, wherein the transcriptional activation domain of ENL comprises the sequence set forth in SEQ ID NO:131.

33. The fusion protein of any of claims 1-32, wherein the transcriptional activation domain of FOXO3 comprises:
(i) the sequence set forth in SEQ ID NO:37;
(ii) a contiguous portion of SEQ ID NO:37 of at least 20 amino acids;
(iii) the sequence set forth in SEQ ID NO:24;
(iv) a contiguous portion of SEQ ID NO:24 of at least 20 amino acids;
(v) an amino acid sequence that has at least 90%, 91%, 92%, 93%, 94%, 95%, 96%, 97%, 98%, or 99% sequence identity to any of the foregoing.

34. The fusion protein of any of claims 1-33, wherein the transcriptional activation domain of FOXO3 comprises the sequence set forth in SEQ ID NO:132 or an amino acid sequence that has at least 90%, 91%, 92%, 93%, 94%, 95%, 96%, 97%, 98%, or 99% sequence identity to any of the foregoing.

35. The fusion protein of any of claims 1-34, wherein the transcriptional activation domain of FOXO3 comprises the sequence set forth in SEQ ID NO:132.

36. The fusion protein of any of claims 1-35, wherein the transcriptional activation domain of PYGO1 comprises:
(i) the sequence set forth in SEQ ID NO:42;
(ii) a contiguous portion of SEQ ID NO:42 of at least 20 amino acids;
(iii) the sequence set forth in SEQ ID NO:29;
(iv) a contiguous portion of SEQ ID NO:29 of at least 20 amino acids;
(v) an amino acid sequence that has at least 90%, 91%, 92%, 93%, 94%, 95%, 96%, 97%, 98%, or 99% sequence identity to any of the foregoing.

37. The fusion protein of any of claims 1-36, wherein the transcriptional activation domain of PYGO1 comprises the sequence set forth in SEQ ID NO:130 or an amino acid sequence that has at least 90%, 91%, 92%, 93%, 94%, 95%, 96%, 97%, 98%, or 99% sequence identity to any of the foregoing.

38. The fusion protein of any of claims 1-37, wherein the transcriptional activation domain of PYGO1 comprises the sequence set forth in SEQ ID NO:130.

39. The fusion protein of any of claims 1-38, wherein the transcriptional activation domain of HSH2D comprises:
(i) the sequence set forth in SEQ ID NO:38;
(ii) a contiguous portion of SEQ ID NO:38 of at least 20 amino acids;
(iii) the sequence set forth in SEQ ID NO:25;
(iv) a contiguous portion of SEQ ID NO:25 of at least 20 amino acids;
(v) an amino acid sequence that has at least 90%, 91%, 92%, 93%, 94%, 95%, 96%, 97%, 98%, or 99% sequence identity to any of the foregoing.

40. The fusion protein of any of claims 1-39, wherein the transcriptional activation domain of HSH2D comprises the sequence set forth in SEQ ID NO:134 or an amino acid sequence that has at least 90%, 91%, 92%, 93%, 94%, 95%, 96%, 97%, 98%, or 99% sequence identity to any of the foregoing.

41. The fusion protein of any of claims 1-40, wherein the transcriptional activation domain of HSH2D comprises the sequence set forth in SEQ ID NO:134.

42. The fusion protein of any of claims 1-41, wherein the transcriptional activation domain of NCOA2 comprises:
(i) the sequence set forth in SEQ ID NO:39;
(ii) a contiguous portion of SEQ ID NO:39 of at least 20 amino acids;
(iii) the sequence set forth in SEQ ID NO:26;
(iv) a contiguous portion of SEQ ID NO:26 of at least 20 amino acids;
(v) an amino acid sequence that has at least 90%, 91%, 92%, 93%, 94%, 95%, 96%, 97%, 98%, or 99% sequence identity to any of the foregoing.

43. The fusion protein of any of claims 1-42, wherein the transcriptional activation domain of NCOA2 comprises the sequence set forth in SEQ ID NO:135 or an amino acid sequence that has at least 90%, 91%, 92%, 93%, 94%, 95%, 96%, 97%, 98%, or 99% sequence identity to any of the foregoing.

44. The fusion protein of any of claims 1-43, wherein the transcriptional activation domain of NCOA2 comprises the sequence set forth in SEQ ID NO:135.

45. The fusion protein of any of claims 1-44, wherein the transcriptional activation domain of NOTCH2 comprises:
(i) the sequence set forth in SEQ ID NO:46 or 390;
(ii) a contiguous portion of SEQ ID NO:46 or 390 of at least 20 amino acids;
(iii) the sequence set forth in SEQ ID NO:33 or 381;
(iv) a contiguous portion of SEQ ID NO:33 or 381 of at least 20 amino acids;
(v) an amino acid sequence that has at least 90%, 91%, 92%, 93%, 94%, 95%, 96%, 97%, 98%, or 99% sequence identity to any of the foregoing.

46. The fusion protein of any of claims 1-45, wherein the transcriptional activation domain of NOTCH2 comprises the sequence set forth in SEQ ID NO:136 or an amino acid sequence that has at least 90%, 91%, 92%, 93%, 94%, 95%, 96%, 97%, 98%, or 99% sequence identity to any of the foregoing.

47. The fusion protein of any of claims 1-46, wherein the transcriptional activation domain of NOTCH2 comprises the sequence set forth in SEQ ID NO:136.

48. The fusion protein of any of claims 1-47, wherein the transcriptional activation domain of DPOLA comprises:
(i) the sequence set forth in SEQ ID NO:35;
(ii) a contiguous portion of SEQ ID NO:35 of at least 20 amino acids;
(iii) the sequence set forth in SEQ ID NO:22;
(iv) a contiguous portion of SEQ ID NO:22 of at least 20 amino acids;
(v) an amino acid sequence that has at least 90%, 91%, 92%, 93%, 94%, 95%, 96%, 97%, 98%, or 99% sequence identity to any of the foregoing.

49. The fusion protein of any of claims 1-48, wherein the transcriptional activation domain of DPOLA comprises the sequence set forth in SEQ ID NO:176 or an amino acid sequence that has at least 90%, 91%, 92%, 93%, 94%, 95%, 96%, 97%, 98%, or 99% sequence identity to any of the foregoing.

50. The fusion protein of any of claims 1-49, wherein the transcriptional activation domain of DPOLA comprises the sequence set forth in SEQ ID NO:176.

51. The fusion protein of any of claims 1-50, wherein the transcriptional activation domain of PSA1 comprises:
(i) the sequence set forth in SEQ ID NO:41;
(ii) a contiguous portion of SEQ ID NO:41 of at least 20 amino acids;
(iii) the sequence set forth in SEQ ID NO:28;
(iv) a contiguous portion of SEQ ID NO:28 of at least 20 amino acids;
(v) an amino acid sequence that has at least 90%, 91%, 92%, 93%, 94%, 95%, 96%, 97%, 98%, or 99% sequence identity to any of the foregoing.

52. The fusion protein of any of claims 1-51, wherein the transcriptional activation domain of PSA1 comprises the sequence set forth in SEQ ID NO:177 or an amino acid sequence that has at least 90%, 91%, 92%, 93%, 94%, 95%, 96%, 97%, 98%, or 99% sequence identity to any of the foregoing.

53. The fusion protein of any of claims 1-52, wherein the transcriptional activation domain of PSA1 comprises the sequence set forth in SEQ ID NO:177.

54. The fusion protein of any of claims 1-53, wherein the transcriptional activation domain of RBM39 comprises:
(i) the sequence set forth in SEQ ID NO:43;
(ii) a contiguous portion of SEQ ID NO:43 of at least 20 amino acids;
(iii) the sequence set forth in SEQ ID NO:30;
(iv) a contiguous portion of SEQ ID NO:30 of at least 20 amino acids;
(v) an amino acid sequence that has at least 90%, 91%, 92%, 93%, 94%, 95%, 96%, 97%, 98%, or 99% sequence identity to any of the foregoing.

55. The fusion protein of any of claims 1-54, wherein the transcriptional activation domain of RBM39 comprises the sequence set forth in SEQ ID NO:178 or an amino acid sequence that has at least 90%, 91%, 92%, 93%, 94%, 95%, 96%, 97%, 98%, or 99% sequence identity to any of the foregoing.

56. The fusion protein of any of claims 1-55, wherein the transcriptional activation domain of RBM39 comprises the sequence set forth in SEQ ID NO:178.

57. The fusion protein of any of claims 1-56, wherein the transcriptional activation domain of HERC2 comprises:
(i) the sequence set forth in SEQ ID NO:44;
(ii) a contiguous portion of SEQ ID NO:44 of at least 20 amino acids;
(iii) the sequence set forth in SEQ ID NO:31;
(iv) a contiguous portion of SEQ ID NO:31 of at least 20 amino acids;
(v) an amino acid sequence that has at least 90%, 91%, 92%, 93%, 94%, 95%, 96%, 97%, 98%, or 99% sequence identity to any of the foregoing.

58. The fusion protein of any of claims 1-57, wherein the transcriptional activation domain of HERC2 comprises the sequence set forth in SEQ ID NO:179 or an amino acid sequence that has at least 90%, 91%, 92%, 93%, 94%, 95%, 96%, 97%, 98%, or 99% sequence identity to any of the foregoing.

59. The fusion protein of any of claims 1-58, wherein the transcriptional activation domain of HERC2 comprises the sequence set forth in SEQ ID NO:179.

60. The fusion protein of any of claims 1-59, wherein the transcriptional activation domain of ZNF473 comprises:
(i) the sequence set forth in SEQ ID NO:387;
(ii) a contiguous portion of SEQ ID NO:387 of at least 20 amino acids;
(iii) the sequence set forth in SEQ ID NO:378;
(iv) a contiguous portion of SEQ ID NO:378 of at least 20 amino acids;
(v) an amino acid sequence that has at least 90%, 91%, 92%, 93%, 94%, 95%, 96%, 97%, 98%, or 99% sequence identity to any of the foregoing.

61. The fusion protein of any of claims 1-60, wherein the transcriptional activation domain of ANM2 comprises:
(i) the sequence set forth in SEQ ID NO:388;
(ii) a contiguous portion of SEQ ID NO:388 of at least 20 amino acids;
(iii) the sequence set forth in SEQ ID NO:379;
(iv) a contiguous portion of SEQ ID NO:379 of at least 20 amino acids;
(v) an amino acid sequence that has at least 90%, 91%, 92%, 93%, 94%, 95%, 96%, 97%, 98%, or 99% sequence identity to any of the foregoing.

62. The fusion protein of any of claims 1-61, wherein the transcriptional activation domain of KIBRA comprises:
(i) the sequence set forth in SEQ ID NO:389;
(ii) a contiguous portion of SEQ ID NO:389 of at least 20 amino acids;
(iii) the sequence set forth in SEQ ID NO:380;
(iv) a contiguous portion of SEQ ID NO:380 of at least 20 amino acids;
(v) an amino acid sequence that has at least 90%, 91%, 92%, 93%, 94%, 95%, 96%, 97%, 98%, or 99% sequence identity to any of the foregoing.

63. The fusion protein of any of claims 1-62, wherein the transcriptional activation domain of IKKA comprises:
(i) the sequence set forth in SEQ ID NO:391;
(ii) a contiguous portion of SEQ ID NO:391 of at least 20 amino acids;
(iii) the sequence set forth in SEQ ID NO:382;
(iv) a contiguous portion of SEQ ID NO:382 of at least 20 amino acids;
(v) an amino acid sequence that has at least 90%, 91%, 92%, 93%, 94%, 95%, 96%, 97%, 98%, or 99% sequence identity to any of the foregoing.

64. The fusion protein of any of claims 1-63, wherein the transcriptional activation domain of APBB1 comprises:
(i) the sequence set forth in SEQ ID NO:392;
(ii) a contiguous portion of SEQ ID NO:392 of at least 20 amino acids;
(iii) the sequence set forth in SEQ ID NO:383;
(iv) a contiguous portion of SEQ ID NO:383 of at least 20 amino acids;
(v) an amino acid sequence that has at least 90%, 91%, 92%, 93%, 94%, 95%, 96%, 97%, 98%, or 99% sequence identity to any of the foregoing.

65. The fusion protein of any of claims 1-64, wherein the transcriptional activation domain of SMN2 comprises:
(i) the sequence set forth in SEQ ID NO:393;
(ii) a contiguous portion of SEQ ID NO:393 of at least 20 amino acids;
(iii) the sequence set forth in SEQ ID NO:384;
(iv) a contiguous portion of SEQ ID NO:384 of at least 20 amino acids;
(v) an amino acid sequence that has at least 90%, 91%, 92%, 93%, 94%, 95%, 96%, 97%, 98%, or 99% sequence identity to any of the foregoing.

66. The fusion protein of any of claims 1-65, wherein the transcriptional activation domain of SERTAD2 comprises:
(i) the sequence set forth in SEQ ID NO:394;
(ii) a contiguous portion of SEQ ID NO:394 of at least 20 amino acids;
(iii) the sequence set forth in SEQ ID NO:385;
(iv) a contiguous portion of SEQ ID NO:385 of at least 20 amino acids;
(v) an amino acid sequence that has at least 90%, 91%, 92%, 93%, 94%, 95%, 96%, 97%, 98%, or 99% sequence identity to any of the foregoing.

67. The fusion protein of any of claims 1-66, wherein the transcriptional activation domain of MYBA comprises:
(i) the sequence set forth in SEQ ID NO:395;
(ii) a contiguous portion of SEQ ID NO:395 of at least 20 amino acids;
(iii) the sequence set forth in SEQ ID NO:386;
(iv) a contiguous portion of SEQ ID NO:386 of at least 20 amino acids;
(v) an amino acid sequence that has at least 90%, 91%, 92%, 93%, 94%, 95%, 96%, 97%, 98%, or 99% sequence identity to any of the foregoing.

68. The fusion protein of any of claims 1-67, wherein the transcriptional activation domain is at least at or about 30, 40, 50, 60, or 70 amino acids in length.

69. The fusion protein of any of claims 1-68, wherein the transcriptional activation domain is at least at or about 40 amino acids in length.

70. The fusion protein of any of claims 1-68, wherein the transcriptional activation domain is at least at or about 50 amino acids in length.

71. The fusion protein of any of claims 1-68, wherein the transcriptional activation domain is at least at or about 60 amino acids in length.

72. The fusion protein of any of claims 1-68, wherein the transcriptional activation domain is at least at or about 70 amino acids in length.

73. The fusion protein of any of claims 1-68, wherein the transcriptional activation domain is at or about 120, 110, 100, 90, 80, 70, 60, 50, or 40 amino acids or less in length.

74. The fusion protein of any of claims 1-68 and 73, wherein the transcriptional activation domain is 70 amino acids or less in length.

75. The fusion protein of any of claims 1-60 and 73, wherein the transcriptional activation domain is 60 amino acids or less in length.

76. The fusion protein of any of claims 1-60 and 73, wherein the transcriptional activation domain is 50 amino acids or less in length.

77. The fusion protein of any of claims 1-67, wherein the transcriptional activation domain is between at or about 40 and at or about 120, at or about 40 and at or about 110, at or about 40 and at or about 100, at or about 40 and at or about 90, at or about 40 and at or about 80, at or about 40 and at or about 70, at or about 40 and at or about 60, or at or about 40 and at or about 50 amino acids in length.

78. The fusion protein of any of claims 1-77, wherein the fusion protein comprises a multipartite effector comprising at least two of the two or more transcriptional activation domains.

79. The fusion protein of claim 78, wherein the multipartite effector is composed of two transcriptional activation domains.

80. The fusion protein of claim 78 or claim 79, wherein the multipartite effector is set forth in any of SEQ ID NOS:140-153, a portion thereof, or an amino acid sequence that has at least 90%, 91%, 92%, 93%, 94%, 95%, 96%, 97%, 98%, or 99% sequence identity to any of the foregoing.

81. The fusion protein of any of claims 1-78, wherein the two or more transcriptional activation domains is two transcriptional activation domains.

82. The fusion protein of any of claims 1-81, wherein the fusion protein comprises:
a transcriptional activation domain of PYGO1 and a transcriptional activation domain of NCOA3;
a transcriptional activation domain of NOTCH2 and a transcriptional activation domain of NCOA3;
a transcriptional activation domain of NCOA3 and a transcriptional activation domain of NCOA3;
a transcriptional activation domain of HSH2D and a transcriptional activation domain of NCOA3;
a transcriptional activation domain of FOXO3 and a transcriptional activation domain of NCOA3;
a transcriptional activation domain of NCOA2 and a transcriptional activation domain of NCOA3;
a transcriptional activation domain of ENL and a transcriptional activation domain of NCOA3;
a transcriptional activation domain of PYGO1 and a transcriptional activation domain of FOXO3;
a transcriptional activation domain of NOTCH2 and a transcriptional activation domain of FOXO3;
a transcriptional activation domain of NCOA3 and a transcriptional activation domain of FOXO3;
a transcriptional activation domain of HSH2D and a transcriptional activation domain of FOXO3;
a transcriptional activation domain of FOXO3 and a transcriptional activation domain of FOXO3;
a transcriptional activation domain of NCOA2 and a transcriptional activation domain of FOXO3;
a transcriptional activation domain of ENL and a transcriptional activation domain of FOXO3;
a transcriptional activation domain of MYBA and a transcriptional activation domain of FOXO3; or
a transcriptional activation domain of SERTAD2 and a transcriptional activation domain of NCOA2.

83. The fusion protein of any of claims 1-82, wherein the fusion protein comprises, in N-terminus to C-terminus order:
a transcriptional activation domain of PYGO1, a linker, and a transcriptional activation domain of NCOA3;
a transcriptional activation domain of NOTCH2, a linker, and a transcriptional activation domain of NCOA3;
a transcriptional activation domain of NCOA3, a linker, and a transcriptional activation domain of NCOA3;
a transcriptional activation domain of HSH2D, a linker, and a transcriptional activation domain of NCOA3;
a transcriptional activation domain of FOXO3, a linker, and a transcriptional activation domain of NCOA3;
a transcriptional activation domain of NCOA2, a linker, and a transcriptional activation domain of NCOA3;
a transcriptional activation domain of ENL, a linker, and a transcriptional activation domain of NCOA3;
a transcriptional activation domain of PYGO1, a linker, and a transcriptional activation domain of FOXO3;
a transcriptional activation domain of NOTCH2, a linker, and a transcriptional activation domain of FOXO3;
a transcriptional activation domain of NCOA3, a linker, and a transcriptional activation domain of FOXO3;
a transcriptional activation domain of HSH2D, a linker, and a transcriptional activation domain of FOXO3;
a transcriptional activation domain of FOXO3, a linker, and a transcriptional activation domain of FOXO3;
a transcriptional activation domain of NCOA2, a linker, and a transcriptional activation domain of FOXO3; or
a transcriptional activation domain of ENL, a linker, and a transcriptional activation domain of FOXO3.

84. The fusion protein of any of claims 1-82, wherein the fusion protein comprises the sequence set forth in any one of SEQ ID NOS:140-153, or an amino acid sequence that has at least 90%, 91%, 92%, 93%, 94%, 95%, 96%, 97%, 98%, or 99% sequence identity to any of the foregoing, optionally wherein the fusion protein comprises the sequence set forth in SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, or SEQ ID NO:153.

85. The fusion protein of any of claims 1-84, wherein the fusion protein comprises, in N-terminus to C-terminus order:
a dCas, optionally a dCas9, a transcriptional activation domain of PYGO1 and a transcriptional activation domain of NCOA3;
a dCas, optionally a dCas9, a transcriptional activation domain of NOTCH2 and a transcriptional activation domain of NCOA3;
a dCas, optionally a dCas9, a transcriptional activation domain of NCOA3 and a transcriptional activation domain of NCOA3;
a dCas, optionally a dCas9, a transcriptional activation domain of HSH2D and a transcriptional activation domain of NCOA3;
a dCas, optionally a dCas9, a transcriptional activation domain of FOXO3 and a transcriptional activation domain of NCOA3;
a dCas, optionally a dCas9, a transcriptional activation domain of NCOA2 and a transcriptional activation domain of NCOA3;
a dCas, optionally a dCas9, a transcriptional activation domain of ENL and a transcriptional activation domain of NCOA3;
a dCas, optionally a dCas9, a transcriptional activation domain of PYGO1 and a transcriptional activation domain of FOXO3;
a dCas, optionally a dCas9, a transcriptional activation domain of NOTCH2 and a transcriptional activation domain of FOXO3;
a dCas, optionally a dCas9, a transcriptional activation domain of NCOA3 and a transcriptional activation domain of FOXO3;
a dCas, optionally a dCas9, a transcriptional activation domain of HSH2D and a transcriptional activation domain of FOXO3;
a dCas, optionally a dCas9, a transcriptional activation domain of FOXO3 and a transcriptional activation domain of FOXO3;
a dCas, optionally a dCas9, a transcriptional activation domain of NCOA2 and a transcriptional activation domain of FOXO3; or
a dCas, optionally a dCas9, a transcriptional activation domain of ENL and a transcriptional activation domain of FOXO3.

86. The fusion protein of any of claims 1-84, wherein the fusion protein comprises, in N-terminus to C-terminus order:
a ZFP, a transcriptional activation domain of PYGO1 and a transcriptional activation domain of NCOA3;
a ZFP, a transcriptional activation domain of NOTCH2 and a transcriptional activation domain of NCOA3;
a ZFP, a transcriptional activation domain of NCOA3 and a transcriptional activation domain of NCOA3;
a ZFP, a transcriptional activation domain of HSH2D and a transcriptional activation domain of NCOA3;
a ZFP, a transcriptional activation domain of FOXO3 and a transcriptional activation domain of NCOA3;
a ZFP, a transcriptional activation domain of NCOA2 and a transcriptional activation domain of NCOA3;
a ZFP, a transcriptional activation domain of ENL and a transcriptional activation domain of NCOA3;
a ZFP, a transcriptional activation domain of PYGO1 and a transcriptional activation domain of FOXO3;
a ZFP, a transcriptional activation domain of NOTCH2 and a transcriptional activation domain of FOXO3;
a ZFP, a transcriptional activation domain of NCOA3 and a transcriptional activation domain of FOXO3;
a ZFP, a transcriptional activation domain of HSH2D and a transcriptional activation domain of FOXO3;
a ZFP, a transcriptional activation domain of FOXO3 and a transcriptional activation domain of FOXO3;
a ZFP, a transcriptional activation domain of NCOA2 and a transcriptional activation domain of FOXO3; or
a ZFP, a transcriptional activation domain of ENL and a transcriptional activation domain of FOXO3.

87. The fusion protein of any of claims 1-84, wherein the fusion protein comprises, in N-terminus to C-terminus order:
a transcriptional activation domain of PYGO1, a transcriptional activation domain of NCOA3, and a dCas, optionally a dCas9;
a transcriptional activation domain of NOTCH2, a transcriptional activation domain of NCOA3, and a dCas, optionally a dCas9;
a transcriptional activation domain of NCOA3, a transcriptional activation domain of NCOA3, and a dCas, optionally a dCas9;
a transcriptional activation domain of HSH2D, a transcriptional activation domain of NCOA3, and a dCas, optionally a dCas9;
a transcriptional activation domain of FOXO3, a transcriptional activation domain of NCOA3, and a dCas, optionally a dCas9;
a transcriptional activation domain of NCOA2, a transcriptional activation domain of NCOA3, and a dCas, optionally a dCas9;
a transcriptional activation domain of ENL, a transcriptional activation domain of NCOA3, and a dCas, optionally a dCas9;
a transcriptional activation domain of PYGO1, a transcriptional activation domain of FOXO3, and a dCas, optionally a dCas9;
a transcriptional activation domain of NOTCH2, a transcriptional activation domain of FOXO3, and a dCas, optionally a dCas9;
a transcriptional activation domain of NCOA3, a transcriptional activation domain of FOXO3, and a dCas, optionally a dCas9;
a transcriptional activation domain of HSH2D, a transcriptional activation domain of FOXO3, and a dCas, optionally a dCas9;
a transcriptional activation domain of FOXO3, a transcriptional activation domain of FOXO3, and a dCas, optionally a dCas9;
a transcriptional activation domain of NCOA2, a transcriptional activation domain of FOXO3, and a dCas, optionally a dCas9; or
a transcriptional activation domain of ENL, a transcriptional activation domain of FOXO3, and a dCas, optionally a dCas9.

88. The fusion protein of any of claims 1-84, wherein the fusion protein comprises, in N-terminus to C-terminus order:
a transcriptional activation domain of PYGO1, a transcriptional activation domain of NCOA3, and a ZFP;
a transcriptional activation domain of NOTCH2, a transcriptional activation domain of NCOA3, and a ZFP;
a transcriptional activation domain of NCOA3, a transcriptional activation domain of NCOA3, and a ZFP;
a transcriptional activation domain of HSH2D, a transcriptional activation domain of NCOA3, and a ZFP;
a transcriptional activation domain of FOXO3, a transcriptional activation domain of NCOA3, and a ZFP;
a transcriptional activation domain of NCOA2, a transcriptional activation domain of NCOA3, and a ZFP;
a transcriptional activation domain of ENL, a transcriptional activation domain of NCOA3, and a ZFP;
a transcriptional activation domain of PYGO1, a transcriptional activation domain of FOXO3, and a ZFP;
a transcriptional activation domain of NOTCH2, a transcriptional activation domain of FOXO3, and a ZFP;
a transcriptional activation domain of NCOA3, a transcriptional activation domain of FOXO3, and a ZFP;
a transcriptional activation domain of HSH2D, a transcriptional activation domain of FOXO3, and a ZFP;
a transcriptional activation domain of FOXO3, a transcriptional activation domain of FOXO3, and a ZFP;
a transcriptional activation domain of NCOA2, a transcriptional activation domain of FOXO3, and a ZFP; or
a transcriptional activation domain of ENL, a transcriptional activation domain of FOXO3, and a ZFP.

89. The fusion protein of any of claims 1-78 and 82-88, wherein the two or more transcriptional activation domains is three transcriptional activation domains.

90. The fusion protein of any of claims 1-78 and 89, wherein the fusion protein comprises a multipartite effector comprising at least three transcriptional activation domains.

91. The fusion protein of claim 78 or 90, wherein the multipartite effector is composed of two transcriptional activation domains or three transcriptional activation domains.

92. The fusion protein of claim 78, 90 or 91, wherein the multipartite effector is set forth in any of SEQ ID NOS:154-160 and 377, a portion thereof, or an amino acid sequence that has at least 90%, 91%, 92%, 93%, 94%, 95%, 96%, 97%, 98%, or 99% sequence identity to any of the foregoing

93. The fusion protein of any of claims 1-78 and 82-92, wherein the fusion protein comprises:
a transcriptional activation domain of PYGO1, a transcriptional activation domain of FOXO3, and a transcriptional activation domain of NCOA3;
a transcriptional activation domain of NOTCH2, a transcriptional activation domain of FOXO3, and a transcriptional activation domain of NCOA3;
a transcriptional activation domain of NCOA3, a transcriptional activation domain of FOXO3, and a transcriptional activation domain of NCOA3;
a transcriptional activation domain of HSH2D, a transcriptional activation domain of FOXO3, and a transcriptional activation domain of NCOA3;
a transcriptional activation domain of FOXO3, a transcriptional activation domain of FOXO3, and a transcriptional activation domain of NCOA3;
a transcriptional activation domain of NCOA2, a transcriptional activation domain of FOXO3, and a transcriptional activation domain of NCOA3;
a transcriptional activation domain of ENL, a transcriptional activation domain of FOXO3, and a transcriptional activation domain of NCOA3; or
a transcriptional activation domain of NCOA3, a transcriptional activation domain of FOXO3, and a transcriptional activation domain of FOXO3.

94. The fusion protein of any of claims 1-78 and 82-93, wherein the fusion protein comprises, in N-terminus to C-terminus order:
a transcriptional activation domain of PYGO1, a linker, a transcriptional activation domain of FOXO3, a linker, and a transcriptional activation domain of NCOA3;
a transcriptional activation domain of NOTCH2, a linker, a transcriptional activation domain of FOXO3, a linker, and a transcriptional activation domain of NCOA3;
a transcriptional activation domain of NCOA3, a linker, a transcriptional activation domain of FOXO3, a linker, and a transcriptional activation domain of NCOA3;
a transcriptional activation domain of HSH2D, a linker, a transcriptional activation domain of FOXO3, a linker, and a transcriptional activation domain of NCOA3;
a transcriptional activation domain of FOXO3, a linker, a transcriptional activation domain of FOXO3, a linker, and a transcriptional activation domain of NCOA3;
a transcriptional activation domain of NCOA2, a linker, a transcriptional activation domain of FOXO3, a linker, and a transcriptional activation domain of NCOA3;
a transcriptional activation domain of ENL, a linker, a transcriptional activation domain of FOXO3, a linker, and a transcriptional activation domain of NCOA3; or
a transcriptional activation domain of NCOA3, a linker, a transcriptional activation domain of FOXO3, a linker, and a transcriptional activation domain of FOXO3.

95. The fusion protein of any of claims 1-78 and 82-94, wherein the fusion protein comprises the sequence set forth in any one of SEQ ID NOS:154-160 and 377 or an amino acid sequence that has at least 90%, 91%, 92%, 93%, 94%, 95%, 96%, 97%, 98%, or 99% sequence identity to any of the foregoing.

96. The fusion protein of any of claims 1-78 and 82-95, wherein the fusion protein comprises the sequence set forth in SEQ ID NO:154, SEQ ID NO:155, SEQ ID NO: 156, SEQ ID NO:157, SEQ ID NO:158, SEQ ID NO:159, or SEQ ID NO:160 or SEQ ID NO: 377.

97. The fusion protein of any of claims 78 and 90-96, wherein the multipartite effector comprises, in the N-terminal to C-terminal direction, domains from FOXO3, FOXO3, and NCOA3, respectively.

98. The fusion protein of claim 97, wherein the multipartite effector comprises the sequence set forth in SEQ ID NO:158, or an amino acid sequence that has at least 90%, 91%, 92%, 93%, 94%, 95%, 96%, 97%, 98%, or 99% sequence identity thereto.

99. The fusion protein of any of claims 78 and 90-96, wherein the multipartite effector comprises, in the N-terminal to C-terminal direction, domains from NCOA3, FOXO3, and NCOA3, respectively

100. The fusion protein of claim 99, wherein the multipartite effector comprises the sequence set forth in SEQ ID NO:156, or an amino acid sequence that has at least 90%, 91%, 92%, 93%, 94%, 95%, 96%, 97%, 98%, or 99% sequence identity thereto

101. The fusion protein of any of claims 78 and 90-96, wherein the multipartite effector comprises, in the N-terminal to C-terminal direction, domains from NCOA2, FOXO3, and NCOA3, respectively

102. The fusion protein of claim 101, wherein the multipartite effector comprises the sequence set forth in SEQ ID NO:159, or an amino acid sequence that has at least 90%, 91%, 92%, 93%, 94%, 95%, 96%, 97%, 98%, or 99% sequence identity thereto

103. The fusion protein of any of claims 78 and 90-96, wherein the multipartite effector comprises, in the N-terminal to C-terminal direction, domains from PYGO1, FOXO3, and NCOA3, respectively

104. The fusion protein of claim 103, wherein the multipartite effector comprises the sequence set forth in SEQ ID NO:154, or an amino acid sequence that has at least 90%, 91%, 92%, 93%, 94%, 95%, 96%, 97%, 98%, or 99% sequence identity thereto

105. The fusion protein of any of claims 78 and 90-96, wherein the multipartite effector comprises, in the N-terminal to C-terminal direction, domains from NCOA3, FOXO3, and FOXO3, respectively

106. The fusion protein of claim 105, wherein the multipartite effector comprises the sequence set forth in SEQ ID NO:377, or an amino acid sequence that has at least 90%, 91%, 92%, 93%, 94%, 95%, 96%, 97%, 98%, or 99% sequence identity thereto

107. The fusion protein of any of claims 1-78 and 82-96, wherein the fusion protein comprises a transcriptional activation domain of PYGO1, a linker, a transcriptional activation domain of FOXO3, a linker, and a transcriptional activation domain of NCOA3.

108. The fusion protein of any of claims 1-78, 82-96, 103, 104, and 107, wherein the fusion protein comprises the sequence set forth in SEQ ID NO:154.

109. The fusion protein of any of claims 1-78 and 82-96, wherein the fusion protein comprises a transcriptional activation domain of NCOA3, a linker, a transcriptional activation domain of FOXO3, a linker, and a transcriptional activation domain of NCOA3.

110. The fusion protein of any of claims 1-78, 82-96, 99, 100, and 109, wherein the fusion protein comprises the sequence set forth in SEQ ID NO:156.

111. The fusion protein of any of claims 1-78, and 82-96, wherein the fusion protein comprises a transcriptional activation domain of FOXO3, a linker, a transcriptional activation domain of FOXO3, a linker, and a transcriptional activation domain of NCOA3.

112. The fusion protein of any of claims 1-78, 82-96, 97, 98 and 111, wherein the fusion protein comprises the sequence set forth in SEQ ID NO:158.

113. The fusion protein of any of claims 1-78 and 82-96, wherein the fusion protein comprises a transcriptional activation domain of NCOA2, a linker, a transcriptional activation domain of FOXO3, a linker, and a transcriptional activation domain of NCOA3.

114. The fusion protein of any of claims 1-78, 82-96, 101, 102 and 113, wherein the fusion protein comprises the sequence set forth in SEQ ID NO:159.

115. The fusion protein of any of claims 1-78 and 82-96, wherein the fusion protein comprises, in N-terminus to C-terminus order:
a dCas, optionally a dCas9, a linker, a transcriptional activation domain of PYGO1, a linker, a transcriptional activation domain of FOXO3, a linker, and a transcriptional activation domain of NCOA3;
a dCas, optionally a dCas9, a linker, a transcriptional activation domain of NOTCH2, a linker, a transcriptional activation domain of FOXO3, a linker, and a transcriptional activation domain of NCOA3;
a dCas, optionally a dCas9, a linker, a transcriptional activation domain of NCOA3, a linker, a transcriptional activation domain of FOXO3, a linker, and a transcriptional activation domain of NCOA3;
a dCas, optionally a dCas9, a linker, a transcriptional activation domain of HSH2D, a linker, a transcriptional activation domain of FOXO3, a linker, and a transcriptional activation domain of NCOA3;
a dCas, optionally a dCas9, a linker, a transcriptional activation domain of FOXO3, a linker, a transcriptional activation domain of FOXO3, a linker, and a transcriptional activation domain of NCOA3;
a dCas, optionally a dCas9, a linker, a transcriptional activation domain of NCOA2, a linker, a transcriptional activation domain of FOXO3, a linker, and a transcriptional activation domain of NCOA3;
a dCas, optionally a dCas9, a linker, a transcriptional activation domain of ENL, a linker, a transcriptional activation domain of FOXO3, a linker, and a transcriptional activation domain of NCOA3; or
a dCas, optionally a dCas9, a linker, a transcriptional activation domain of NCOA3, a linker, a transcriptional activation domain of FOXO3, a linker, and a transcriptional activation domain of FOXO3.

116. The fusion protein of any of claims 1-78 and 82-96, wherein the fusion protein comprises, in N-terminus to C-terminus order:
a ZFP, a linker, a transcriptional activation domain of PYGO1, a linker, a transcriptional activation domain of FOXO3, a linker, and a transcriptional activation domain of NCOA3;
a ZFP, a linker, a transcriptional activation domain of NOTCH2, a linker, a transcriptional activation domain of FOXO3, a linker, and a transcriptional activation domain of NCOA3;
a ZFP, a linker, a transcriptional activation domain of NCOA3, a linker, a transcriptional activation domain of FOXO3, a linker, and a transcriptional activation domain of NCOA3;
a ZFP, a linker, a transcriptional activation domain of HSH2D, a linker, a transcriptional activation domain of FOXO3, a linker, and a transcriptional activation domain of NCOA3;
a ZFP, a linker, a transcriptional activation domain of FOXO3, a linker, a transcriptional activation domain of FOXO3, a linker, and a transcriptional activation domain of NCOA3;
a ZFP, a linker, a transcriptional activation domain of NCOA2, a linker, a transcriptional activation domain of FOXO3, a linker, and a transcriptional activation domain of NCOA3;
a ZFP, a linker, a transcriptional activation domain of ENL, a linker, a transcriptional activation domain of FOXO3, a linker, and a transcriptional activation domain of NCOA3; or
a ZFP, a linker, a transcriptional activation domain of NCOA3, a linker, a transcriptional activation domain of FOXO3, a linker, and a transcriptional activation domain of FOXO3.

117. The fusion protein of any of claims 1-78 and 82-96, wherein the fusion protein comprises, in N-terminus to C-terminus order:
a transcriptional activation domain of PYGO1, a linker, a transcriptional activation domain of FOXO3, a linker, a transcriptional activation domain of NCOA3, a linker, and a dCas, optionally a dCas9;
a transcriptional activation domain of NOTCH2, a linker, a transcriptional activation domain of FOXO3, a linker, a transcriptional activation domain of NCOA3, a linker, and a dCas, optionally a dCas9;
a transcriptional activation domain of NCOA3, a linker, a transcriptional activation domain of FOXO3, a linker, a transcriptional activation domain of NCOA3, a linker, and a dCas, optionally a dCas9;
a transcriptional activation domain of HSH2D, a linker, a transcriptional activation domain of FOXO3, a linker, a transcriptional activation domain of NCOA3, a linker, and a dCas, optionally a dCas9;
a transcriptional activation domain of FOXO3, a linker, a transcriptional activation domain of FOXO3, a linker, a transcriptional activation domain of NCOA3, a linker, and a dCas, optionally a dCas9;
a transcriptional activation domain of NCOA2, a linker, a transcriptional activation domain of FOXO3, a linker, a transcriptional activation domain of NCOA3, a linker, and a dCas, optionally a dCas9;
a transcriptional activation domain of ENL, a linker, a transcriptional activation domain of FOXO3, a linker, a transcriptional activation domain of NCOA3, a linker, and a dCas, optionally a dCas9; or
a transcriptional activation domain of NCOA3, a linker, a transcriptional activation domain of FOXO3, a linker, a transcriptional activation domain of FOXO3, a linker, and a dCas, optionally a dCas9.

118. The fusion protein of any of claims 1-78 and 82-96, wherein the fusion protein comprises, in N-terminus to C-terminus order:
a transcriptional activation domain of PYGO1, a linker, a transcriptional activation domain of FOXO3, a linker, a transcriptional activation domain of NCOA3, a linker, and a ZFP;
a transcriptional activation domain of NOTCH2, a linker, a transcriptional activation domain of FOXO3, a linker, a transcriptional activation domain of NCOA3, a linker, and a ZFP;
a transcriptional activation domain of NCOA3, a linker, a transcriptional activation domain of FOXO3, a linker, a transcriptional activation domain of NCOA3, a linker, and a ZFP;
a transcriptional activation domain of HSH2D, a linker, a transcriptional activation domain of FOXO3, a linker, a transcriptional activation domain of NCOA3, a linker, and a ZFP;
a transcriptional activation domain of FOXO3, a linker, a transcriptional activation domain of FOXO3, a linker, a transcriptional activation domain of NCOA3, a linker, and a ZFP;
a transcriptional activation domain of NCOA2, a linker, a transcriptional activation domain of FOXO3, a linker, a transcriptional activation domain of NCOA3, a linker, and a ZFP; or
a transcriptional activation domain of ENL, a linker, a transcriptional activation domain of FOXO3, a linker, a transcriptional activation domain of NCOA3, a linker, and a ZFP; or
a transcriptional activation domain of NCOA3, a linker, a transcriptional activation domain of FOXO3, a linker, a transcriptional activation domain of FOXO3, a linker, and a ZFP.

119. The fusion protein of any of claims 1-78, 82, 83, 93, and 94, wherein the two or more transcriptional activation domains comprises four transcriptional activation domains.

120. The fusion protein of any of claims 1-78, 82, 83, 93, and 94, wherein the two or more transcriptional activation domains comprises five transcriptional activation domains.

121. The fusion protein of any of claims 1-120, wherein the fusion protein further comprises one or more linkers.

122. The fusion protein of claim 121, wherein a linker of the one or more linkers is positioned between the two or more transcriptional activation domains and/or positioned between the polypeptide component of the DNA-targeting domain and one of the two or more transcriptional activation domains.

123. The fusion protein of claim 121 or claim 122, wherein the linker is a polypeptide linker.

124. The fusion protein of claim 123, wherein the polypeptide linker comprises a sequence selected from among SEQ ID NOS:62-67, 96, and 137-139.

125. The fusion protein of any of claims 1-124, wherein the fusion protein further comprises one or more nuclear localization signals (NLSs).

126. The fusion protein of claim 125, wherein the one or more NLSs comprises two or more NLSs.

127. The fusion protein of claim 125 or claim 126, wherein a NLS of one or more NLSs is positioned between the two or more transcriptional activation domains.

128. The fusion protein of any of claims 125-127, wherein a NLS of the one or more NLSs is positioned between the polypeptide component of the DNA-targeting domain and one of the two or more transcriptional activation domains.

129. The fusion protein of any of claims 125-128, wherein the one or more NLSs comprises a sequence selected from among SEQ ID NOS:69-84.

130. The fusion protein of any of claims 1-96 and 115, 117 and 119-129, wherein the fusion protein comprises the sequence set forth in any one of SEQ ID NOS:181-187, or an amino acid sequence that has at least 90%, 91%, 92%, 93%, 94%, 95%, 96%, 97%, 98%, or 99% sequence identity to any of the foregoing.

131. The fusion protein of any of claims 1-96 and 115, 117 and 119-130, wherein the fusion protein comprises the sequence set forth in SEQ ID NO:181, SEQ ID NO: 182, SEQ ID NO:183, SEQ ID NO:184, SEQ ID NO:185, SEQ ID NO:186, or SEQ ID NO: 187.

132. The fusion protein of any of claims 1-131, further comprising a tag.

133. The fusion protein of claim 132, wherein the tag comprises an epitope tag or a split protein tag.

134. The fusion protein of claim 132 or claim 133, wherein the tag is selected from among SEQ ID NOS:61, 88, 92, and 167.

135. The fusion protein of any of claims 1-96 and 115, 117 and 119-134, wherein the fusion protein comprises the sequence set forth in any one of SEQ ID NOS:272-278, or an amino acid sequence that has at least 90%, 91%, 92%, 93%, 94%, 95%, 96%, 97%, 98%, or 99% sequence identity to any of the foregoing.

136. The fusion protein of any of claims 1-96 and 115, 117 and 119-135, wherein the fusion protein comprises the sequence set forth in SEQ ID NO:272, SEQ ID NO:273, SEQ ID NO:274, SEQ ID NO:275, SEQ ID NO:276, SEQ ID NO:277, or SEQ ID NO:278.

137. The fusion protein of any of claims 1-136, wherein the DNA-targeting domain is capable of hybridizing to the target site or is complementary to the target site at the endogenous locus.

138. The fusion protein of any of claims 1-137, wherein the at least one gRNA is capable of hybridizing to the target site or is complementary to the target site at the endogenous locus.

139. The fusion protein of claim 137 or 138, wherein the target site is located at a regulatory DNA element of the endogenous locus.

140. The fusion protein of claim 139, wherein the regulatory DNA element is selected from among a promoter, an upstream regulatory element, an enhancer, an exon, an intron, a 5′ untranslated region (UTR), a 3′ UTR, or a downstream regulatory element.

141. The fusion protein of any of claims 1-140, wherein the endogenous locus is in a human cell.

142. The fusion protein of any of claims 1-141, wherein the endogenous locus is in a stem cell; liver cell, optionally a hepatocyte; muscle cell; heart cell, optionally a cardiomyocyte; brain cell, optionally a neuron; blood cell; immune cell, optionally a lymphoid cell, optionally a T cell; or a cell derived from any of the foregoing.

143. The fusion protein of any of claims 1-142, wherein the endogenous locus is FXN.

144. The fusion protein of any of claims 1-143, wherein the target site is located within the genomic coordinates hg38 chr9:68,940,179-69,205,519 or hg38 chr9:69,027,282-69,028,497.

145. The fusion protein of any of claims 1-144, wherein the target site is located within the genomic coordinates hg38 chr9:69,027,615-69,028,101.

146. The fusion protein of any of claims 1-144, wherein the target site comprises a sequence set forth in any one of SEQ ID NOS:208-228.

147. The fusion protein of any of claims 1-144 and 146, wherein the target site comprises a sequence set forth in SEQ ID NO:208.

148. The fusion protein of any of claims 1-144 and 146, wherein the target site comprises a sequence set forth in SEQ ID NO:214.

149. The fusion protein of any of claims 1-146, wherein the target site comprises a sequence set forth in SEQ ID NO:228.

150. A DNA-targeting system comprising the fusion protein of any of claims 1-149.

151. A DNA-targeting system comprising the fusion protein of any of claims 1-150, and at least one gRNA.

152. A DNA-targeting system comprising:
(1) a DNA-targeting domain, and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2, wherein the transcriptional activation domain increases transcription of an endogenous locus when recruited to a target site at the endogenous locus.

153. The DNA-targeting system of claim 152, wherein the DNA-targeting domain comprises a Cas-gRNA combination comprising a Cas protein or a variant thereof, and at least one gRNA that binds to the target site at the endogenous locus.

154. The DNA-targeting system of claim 153, wherein the DNA-targeting domain comprises a zinc finger protein (ZFP), a transcription activator-like effector (TALE), a meganuclease, a homing endonuclease, or an I-SceI enzyme or a variant thereof that binds to the target site at the endogenous locus.

155. A DNA-targeting system comprising:
(1) a Cas-gRNA combination comprising a Cas protein or a variant thereof, and at least one gRNA that binds to the target site at an endogenous locus, and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2, wherein the transcriptional activation domain increases transcription of an endogenous locus when recruited to a target site at the endogenous locus.

156. A DNA-targeting system comprising:
(1) a zinc finger protein (ZFP), a transcription activator-like effector (TALE), a meganuclease, a homing endonuclease, or an I-SceI enzyme or a variant thereof that binds to the target site at an endogenous locus, and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2, wherein the transcriptional activation domain increases transcription of an endogenous locus when recruited to a target site at the endogenous locus.

157. A DNA-targeting system comprising:
(1) a zinc finger protein (ZFP) that binds to the target site at an endogenous locus; and
(2) two or more transcriptional activation domains, each transcriptional activation domain comprising a domain of a protein selected from among NCOA3, ENL, FOXO3, PYGO1, HSH2D, NCOA2, NOTCH2, DPOLA, PSA1, RBM39, ZNF473, ANM2, KIBRA, IKKA, APBB1, SMN2, SERTAD2, MYBA, and HERC2, wherein the transcriptional activation domain increases transcription of an endogenous locus when recruited to a target site at the endogenous locus.

158. The DNA-targeting system of claim 153 or 155, wherein the Cas protein or a variant thereof, and the two or more transcriptional activation domains are fused in a fusion protein.

159. The DNA-targeting system of claim 154 or 156, wherein the zinc finger protein (ZFP), a transcription activator-like effector (TALE), a meganuclease, a homing endonuclease, or an I-SceI enzyme or a variant thereof, and the two or more transcriptional activation domains are fused in a fusion protein.

160. The DNA-targeting system of claim 154 or 157, wherein the zinc finger protein (ZFP) and the two or more transcriptional activation domains are fused in a fusion protein.

161. The DNA-targeting system of any of claims 153-159, wherein the variant thereof comprises a catalytically inactive variant.

162. The DNA-targeting system of any of claims 153, 155, 158, and 161, wherein the Cas protein or a variant thereof is a Cas9 or a variant thereof.

163. The DNA-targeting system of any of claims 153, 155, 158, 161, and 162, wherein the Cas protein or a variant thereof is a deactivated Cas9 (dCas9).

164. The DNA-targeting system of any of claims 153, 155, 158, and 161-163, wherein the Cas protein or a variant thereof is a Staphylococcus aureus Cas9 (SaCas9) or a variant thereof.

165. The DNA-targeting system of any of claims 153, 155, 158, and 161-164, wherein the Cas protein or a variant thereof is a Staphylococcus aureus dCas9 (dSaCas9) that comprises at least one amino acid mutation selected from D10A and N580A, with reference to numbering of positions of SEQ ID NO:3.

166. The DNA-targeting system of any of claims 153, 155, 158, and 161-165, wherein the Cas protein or a variant thereof comprises the sequence set forth in SEQ ID NO:2, or an amino acid sequence that has at least 90%, 91%, 92%, 93%, 94%, 95%, 96%, 97%, 98%, or 99% sequence identity thereto.

167. The DNA-targeting system of any of claims 153, 155, 158, and 161-163, wherein the Cas protein or a variant thereof is a Streptococcus pyogenes Cas9 (SpCas9) or a variant thereof.

168. The DNA-targeting system of any of claims 153, 155, 158, 161-163 and 167, wherein the Cas protein or a variant thereof is a Streptococcus pyogenes dCas9 (dSpCas9) that comprises at least one amino acid mutation selected from D10A and H840A, with reference to numbering of positions of SEQ ID NO:7.

169. The DNA-targeting system of any of claims 153, 155, 158, 161-163, 167 and 168, wherein the Cas protein or a variant thereof comprises the sequence set forth in SEQ ID NO:6, or an amino acid sequence that has at least 90%, 91%, 92%, 93%, 94%, 95%, 96%, 97%, 98%, or 99% sequence identity thereto.

170. The DNA-targeting system of any of claims 153, 155, 158, and 161-169, wherein the Cas protein or a variant thereof protein is a split variant Cas protein, wherein the split variant Cas protein comprises a first polypeptide comprising an N-terminal fragment of the variant Cas protein and an N-terminal Intein, and a second polypeptide comprising a C-terminal fragment of the variant Cas protein and a C-terminal Intein.

171. The DNA-targeting system of claim 170, wherein when the first polypeptide and the second polypeptide of the split variant Cas protein are present in proximity or present in the same cell, the N-terminal Intein and C-terminal Intein self-excise and ligate the N-terminal fragment and the C-terminal fragment of the variant Cas protein to form a full-length variant Cas protein.

172. The DNA-targeting system of claim 170 or 171, wherein the N-terminal Intein comprises an N-terminal Npu Intein, or the sequence set forth in SEQ ID NO:88, or an amino acid sequence that has at least 90%, 91%, 92%, 93%, 94%, 95%, 96%, 97%, 98%, or 99% sequence identity thereto, or a portion of any of the foregoing.

173. The DNA-targeting system of any of claims 170-172, wherein the N-terminal fragment of the variant Cas protein comprises:
the N-terminal fragment of variant SpCas9 from the N-terminal end up to position 573 of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or
the sequence set forth in SEQ ID NO:86, or an amino acid sequence that has at least 90%, 91%, 92%, 93%, 94%, 95%, 96%, 97%, 98%, or 99% sequence identity thereto, or a portion of any of the foregoing.

174. The DNA-targeting system of any of claims 170-173, wherein the C-terminal Intein comprises a C-terminal Npu Intein, or the sequence set forth in SEQ ID NO:92, or an amino acid sequence that has at least 90%, 91%, 92%, 93%, 94%, 95%, 96%, 97%, 98%, or 99% sequence identity thereto, or a portion of any of the foregoing.

175. The DNA-targeting system of any of claims 170-174, wherein the C-terminal fragment of the variant Cas protein comprises:
the C-terminal fragment of variant SpCas9 from position 574 to the C-terminal end of the dSpCas9 sequence set forth in SEQ ID NO:6, or an amino acid sequence that has at least 90%, 91%, 92%, 93%, 94%, 95%, 96%, 97%, 98%, or 99% sequence identity thereto; or
the sequence set forth in SEQ ID NO:94, or an amino acid sequence that has at least 90%, 91%, 92%, 93%, 94%, 95%, 96%, 97%, 98%, or 99% sequence identity thereto, or a portion of any of the foregoing.

176. The DNA-targeting system of any of claims 153, 155, 158, and 161, wherein the Cas protein or a variant thereof is a Cpf1 or a variant thereof.

177. The DNA-targeting system of any of claims 153, 155, 158, 161 and 176, wherein the Cas protein or a variant thereof is a variant Cpf1 that that is a deactivated Cpf1 (dCpf1).

178. The DNA-targeting system of claim 176 or 177, wherein the variant comprises a catalytically inactive nuclease variant.

179. The DNA-targeting system of any of claims 152-178, wherein the transcriptional activation domain of NCOA3 comprises:
(i) the sequence set forth in SEQ ID NO:40;
(ii) a contiguous portion of SEQ ID NO:40 of at least 20 amino acids;
(iii) the sequence set forth in SEQ ID NO:27;
(iv) a contiguous portion of SEQ ID NO:27 of at least 20 amino acids;
(v) an amino acid sequence that has at least 90%, 91%, 92%, 93%, 94%, 95%, 96%, 97%, 98%, or 99% sequence identity to any of the foregoing.

180. The DNA-targeting system of any of claims 152-179, wherein the transcriptional activation domain of NCOA3 comprises the sequence set forth in SEQ ID NO:133 or an amino acid sequence that has at least 90%, 91%, 92%, 93%, 94%, 95%, 96%, 97%, 98%, or 99% sequence identity to SEQ ID NO:133.

181. The DNA-targeting system of any of claims 152-180, wherein the transcriptional activation domain of NCOA3 comprises the sequence set forth in SEQ ID NO:133.

182. The DNA-targeting system of any of claims 152-181, wherein the transcriptional activation domain of ENL comprises:
(i) the sequence set forth in SEQ ID NO:36;
(ii) a contiguous portion of SEQ ID NO:36 of at least 20 amino acids;
(iii) the sequence set forth in SEQ ID NO:23;
(iv) a contiguous portion of SEQ ID NO:23 of at least 20 amino acids;
(v) an amino acid sequence that has at least 90%, 91%, 92%, 93%, 94%, 95%, 96%, 97%, 98%, or 99% sequence identity to any of the foregoing.

183. The DNA-targeting system of any of claims 152-182, wherein the transcriptional activation domain of ENL comprises the sequence set forth in SEQ ID NO:131 or an amino acid sequence that has at least 90%, 91%, 92%, 93%, 94%, 95%, 96%, 97%, 98%, or 99% sequence identity to any of the foregoing.

184. The DNA-targeting system of any of claims 152-183, wherein the transcriptional activation domain of ENL comprises the sequence set forth in SEQ ID NO:131.

185. The DNA-targeting system of any of claims 152-184, wherein the transcriptional activation domain of FOXO3 comprises:
(i) the sequence set forth in SEQ ID NO:37;
(ii) a contiguous portion of SEQ ID NO:37 of at least 20 amino acids;
(iii) the sequence set forth in SEQ ID NO:24;
(iv) a contiguous portion of SEQ ID NO:24 of at least 20 amino acids;
(v) an amino acid sequence that has at least 90%, 91%, 92%, 93%, 94%, 95%, 96%, 97%, 98%, or 99% sequence identity to any of the foregoing.

186. The DNA-targeting system of any of claims 152-185, wherein the transcriptional activation domain of FOXO3 comprises the sequence set forth in SEQ ID NO:132 or an amino acid sequence that has at least 90%, 91%, 92%, 93%, 94%, 95%, 96%, 97%, 98%, or 99% sequence identity to any of the foregoing.

187. The DNA-targeting system of any of claims 152-186, wherein the transcriptional activation domain of FOXO3 comprises the sequence set forth in SEQ ID NO:132.

188. The DNA-targeting system of any of claims 152-187, wherein the transcriptional activation domain of PYGO1 comprises:
(i) the sequence set forth in SEQ ID NO:42;
(ii) a contiguous portion of SEQ ID NO:42 of at least 20 amino acids;
(iii) the sequence set forth in SEQ ID NO:29;
(iv) a contiguous portion of SEQ ID NO:29 of at least 20 amino acids;
(v) an amino acid sequence that has at least 90%, 91%, 92%, 93%, 94%, 95%, 96%, 97%, 98%, or 99% sequence identity to any of the foregoing.

189. The DNA-targeting system of any of claims 152-188, wherein the transcriptional activation domain of PYGO1 comprises the sequence set forth in SEQ ID NO:130 or an amino acid sequence that has at least 90%, 91%, 92%, 93%, 94%, 95%, 96%, 97%, 98%, or 99% sequence identity to any of the foregoing.

190. The DNA-targeting system of any of claims 152-189, wherein the transcriptional activation domain of PYGO1 comprises the sequence set forth in SEQ ID NO:130.

191. The DNA-targeting system of any of claims 152-190, wherein the transcriptional activation domain of HSH2D comprises:
(i) the sequence set forth in SEQ ID NO:38;
(ii) a contiguous portion of SEQ ID NO:38 of at least 20 amino acids;
(iii) the sequence set forth in SEQ ID NO:25;
(iv) a contiguous portion of SEQ ID NO:25 of at least 20 amino acids;
(v) an amino acid sequence that has at least 90%, 91%, 92%, 93%, 94%, 95%, 96%, 97%, 98%, or 99% sequence identity to any of the foregoing.

192. The DNA-targeting system of any of claims 152-191, wherein the transcriptional activation domain of HSH2D comprises the sequence set forth in SEQ ID NO:134 or an amino acid sequence that has at least 90%, 91%, 92%, 93%, 94%, 95%, 96%, 97%, 98%, or 99% sequence identity to any of the foregoing.

193. The DNA-targeting system of any of claims 152-192, wherein the transcriptional activation domain of HSH2D comprises the sequence set forth in SEQ ID NO:134.

194. The DNA-targeting system of any of claims 152-193, wherein the transcriptional activation domain of NCOA2 comprises:
(i) the sequence set forth in SEQ ID NO:39;
(ii) a contiguous portion of SEQ ID NO:39 of at least 20 amino acids;
(iii) the sequence set forth in SEQ ID NO:26;
(iv) a contiguous portion of SEQ ID NO:26 of at least 20 amino acids;
(v) an amino acid sequence that has at least 90%, 91%, 92%, 93%, 94%, 95%, 96%, 97%, 98%, or 99% sequence identity to any of the foregoing.

195. The DNA-targeting system of any of claims 152-194, wherein the transcriptional activation domain of NCOA2 comprises the sequence set forth in SEQ ID NO:135 or an amino acid sequence that has at least 90%, 91%, 92%, 93%, 94%, 95%, 96%, 97%, 98%, or 99% sequence identity to any of the foregoing.

196. The DNA-targeting system of any of claims 152-195, wherein the transcriptional activation domain of NCOA2 comprises the sequence set forth in SEQ ID NO:135.

197. The DNA-targeting system of any of claims 152-196, wherein the transcriptional activation domain of NOTCH2 comprises:
(i) the sequence set forth in SEQ ID NO:46 or 390;
(ii) a contiguous portion of SEQ ID NO:46 or 390 of at least 20 amino acids;
(iii) the sequence set forth in SEQ ID NO:33 or 381;
(iv) a contiguous portion of SEQ ID NO:33 or 381 of at least 20 amino acids;
(v) an amino acid sequence that has at least 90%, 91%, 92%, 93%, 94%, 95%, 96%, 97%, 98%, or 99% sequence identity to any of the foregoing.

198. The DNA-targeting system of any of claims 152-197, wherein the transcriptional activation domain of NOTCH2 comprises the sequence set forth in SEQ ID NO:136 or an amino acid sequence that has at least 90%, 91%, 92%, 93%, 94%, 95%, 96%, 97%, 98%, or 99% sequence identity to any of the foregoing.

199. The DNA-targeting system of any of claims 152-198, wherein the transcriptional activation domain of NOTCH2 comprises the sequence set forth in SEQ ID NO:136.

200. The DNA-targeting system of any of claims 152-199, wherein the transcriptional activation domain of DPOLA comprises:
(i) the sequence set forth in SEQ ID NO:35;
(ii) a contiguous portion of SEQ ID NO:35 of at least 20 amino acids;
(iii) the sequence set forth in SEQ ID NO:22;
(iv) a contiguous portion of SEQ ID NO:22 of at least 20 amino acids;
(v) an amino acid sequence that has at least 90%, 91%, 92%, 93%, 94%, 95%, 96%, 97%, 98%, or 99% sequence identity to any of the foregoing.

201. The DNA-targeting system of any of claims 152-200, wherein the transcriptional activation domain of DPOLA comprises the sequence set forth in SEQ ID NO:176 or an amino acid sequence that has at least 90%, 91%, 92%, 93%, 94%, 95%, 96%, 97%, 98%, or 99% sequence identity to any of the foregoing.

202. The DNA-targeting system of any of claims 152-201, wherein the transcriptional activation domain of DPOLA comprises the sequence set forth in SEQ ID NO:176.

203. The DNA-targeting system of any of claims 152-202, wherein the transcriptional activation domain of PSA1 comprises:
(i) the sequence set forth in SEQ ID NO:41;
(ii) a contiguous portion of SEQ ID NO:41 of at least 20 amino acids;
(iii) the sequence set forth in SEQ ID NO:28;
(iv) a contiguous portion of SEQ ID NO:28 of at least 20 amino acids;
(v) an amino acid sequence that has at least 90%, 91%, 92%, 93%, 94%, 95%, 96%, 97%, 98%, or 99% sequence identity to any of the foregoing.

204. The DNA-targeting system of any of claims 152-203, wherein the transcriptional activation domain of PSA1 comprises the sequence set forth in SEQ ID NO:177 or an amino acid sequence that has at least 90%, 91%, 92%, 93%, 94%, 95%, 96%, 97%, 98%, or 99% sequence identity to any of the foregoing.

205. The DNA-targeting system of any of claims 152-204, wherein the transcriptional activation domain of PSA1 comprises the sequence set forth in SEQ ID NO:177.

206. The DNA-targeting system of any of claims 152-205, wherein the transcriptional activation domain of RBM39 comprises:
(i) the sequence set forth in SEQ ID NO:43;
(ii) a contiguous portion of SEQ ID NO:43 of at least 20 amino acids;
(iii) the sequence set forth in SEQ ID NO:30;
(iv) a contiguous portion of SEQ ID NO:30 of at least 20 amino acids;
(v) an amino acid sequence that has at least 90%, 91%, 92%, 93%, 94%, 95%, 96%, 97%, 98%, or 99% sequence identity to any of the foregoing.

207. The DNA-targeting system of any of claims 152-206, wherein the transcriptional activation domain of RBM39 comprises the sequence set forth in SEQ ID NO:178 or an amino acid sequence that has at least 90%, 91%, 92%, 93%, 94%, 95%, 96%, 97%, 98%, or 99% sequence identity to any of the foregoing.

208. The DNA-targeting system of any of claims 152-207, wherein the transcriptional activation domain of RBM39 comprises the sequence set forth in SEQ ID NO:178.

209. The DNA-targeting system of any of claims 152-208, wherein the transcriptional activation domain of HERC2 comprises:
(i) the sequence set forth in SEQ ID NO:44;
(ii) a contiguous portion of SEQ ID NO:44 of at least 20 amino acids;
(iii) the sequence set forth in SEQ ID NO:31;
(iv) a contiguous portion of SEQ ID NO:31 of at least 20 amino acids;
(v) an amino acid sequence that has at least 90%, 91%, 92%, 93%, 94%, 95%, 96%, 97%, 98%, or 99% sequence identity to any of the foregoing.

210. The DNA-targeting system of any of claims 152-209, wherein the transcriptional activation domain of HERC2 comprises the sequence set forth in SEQ ID NO:179 or an amino acid sequence that has at least 90%, 91%, 92%, 93%, 94%, 95%, 96%, 97%, 98%, or 99% sequence identity to any of the foregoing.

211. The DNA-targeting system of any of claims 152-210, wherein the transcriptional activation domain of HERC2 comprises the sequence set forth in SEQ ID NO:179.

212. The DNA-targeting system of any of claims 152-211, wherein the transcriptional activation domain of ZNF473 comprises:
(i) the sequence set forth in SEQ ID NO:387;
(ii) a contiguous portion of SEQ ID NO:387 of at least 20 amino acids;
(iii) the sequence set forth in SEQ ID NO:378;
(iv) a contiguous portion of SEQ ID NO:378 of at least 20 amino acids;
(v) an amino acid sequence that has at least 90%, 91%, 92%, 93%, 94%, 95%, 96%, 97%, 98%, or 99% sequence identity to any of the foregoing.

213. The DNA-targeting system of any of claims 152-212, wherein the transcriptional activation domain of ANM2 comprises:
(i) the sequence set forth in SEQ ID NO:388;
(ii) a contiguous portion of SEQ ID NO:388 of at least 20 amino acids;
(iii) the sequence set forth in SEQ ID NO:379;
(iv) a contiguous portion of SEQ ID NO:379 of at least 20 amino acids;
(v) an amino acid sequence that has at least 90%, 91%, 92%, 93%, 94%, 95%, 96%, 97%, 98%, or 99% sequence identity to any of the foregoing.

214. The DNA-targeting system of any of claims 152-213, wherein the transcriptional activation domain of KIBRA comprises:
(i) the sequence set forth in SEQ ID NO:389;
(ii) a contiguous portion of SEQ ID NO:389 of at least 20 amino acids;
(iii) the sequence set forth in SEQ ID NO:380;
(iv) a contiguous portion of SEQ ID NO:380 of at least 20 amino acids;
(v) an amino acid sequence that has at least 90%, 91%, 92%, 93%, 94%, 95%, 96%, 97%, 98%, or 99% sequence identity to any of the foregoing.

215. The DNA-targeting system of any of claims 152-214, wherein the transcriptional activation domain of IKKA comprises:
(i) the sequence set forth in SEQ ID NO:391;
(ii) a contiguous portion of SEQ ID NO:391 of at least 20 amino acids;
(iii) the sequence set forth in SEQ ID NO:382;
(iv) a contiguous portion of SEQ ID NO:382 of at least 20 amino acids;
(v) an amino acid sequence that has at least 90%, 91%, 92%, 93%, 94%, 95%, 96%, 97%, 98%, or 99% sequence identity to any of the foregoing.

216. The DNA-targeting system of any of claims 152-216, wherein the transcriptional activation domain of APBB1 comprises:
(i) the sequence set forth in SEQ ID NO:392;
(ii) a contiguous portion of SEQ ID NO:392 of at least 20 amino acids;
(iii) the sequence set forth in SEQ ID NO:383;
(iv) a contiguous portion of SEQ ID NO:383 of at least 20 amino acids;
(v) an amino acid sequence that has at least 90%, 91%, 92%, 93%, 94%, 95%, 96%, 97%, 98%, or 99% sequence identity to any of the foregoing.

217. The DNA-targeting system of any of claims 152-216, wherein the transcriptional activation domain of SMN2 comprises:
(i) the sequence set forth in SEQ ID NO:393;
(ii) a contiguous portion of SEQ ID NO:393 of at least 20 amino acids;
(iii) the sequence set forth in SEQ ID NO:384;
(iv) a contiguous portion of SEQ ID NO:384 of at least 20 amino acids;
(v) an amino acid sequence that has at least 90%, 91%, 92%, 93%, 94%, 95%, 96%, 97%, 98%, or 99% sequence identity to any of the foregoing.

218. The DNA-targeting system of any of claims 152-217, wherein the transcriptional activation domain of SERTAD2 comprises:
(i) the sequence set forth in SEQ ID NO:394;
(ii) a contiguous portion of SEQ ID NO:394 of at least 20 amino acids;
(iii) the sequence set forth in SEQ ID NO:385;
(iv) a contiguous portion of SEQ ID NO:385 of at least 20 amino acids;
(v) an amino acid sequence that has at least 90%, 91%, 92%, 93%, 94%, 95%, 96%, 97%, 98%, or 99% sequence identity to any of the foregoing.

219. The DNA-targeting system of any of claims 152-218, wherein the transcriptional activation domain of MYBA comprises:
(i) the sequence set forth in SEQ ID NO:395;
(ii) a contiguous portion of SEQ ID NO:395 of at least 20 amino acids;
(iii) the sequence set forth in SEQ ID NO:386;
(iv) a contiguous portion of SEQ ID NO:386 of at least 20 amino acids;
(v) an amino acid sequence that has at least 90%, 91%, 92%, 93%, 94%, 95%, 96%, 97%, 98%, or 99% sequence identity to any of the foregoing.

220. The DNA-targeting system of any of claims 152-219, wherein the transcriptional activation domain is at least at or about 30, 40, 50, 60, or 70 amino acids in length.

221. The DNA-targeting system of any of claims 152-220, wherein the transcriptional activation domain is at least at or about 40 amino acids in length.

222. The DNA-targeting system of any of claims 152-220, wherein the transcriptional activation domain is at least at or about 50 amino acids in length.

223. The DNA-targeting system of any of claims 152-220, wherein the transcriptional activation domain is at least at or about 60 amino acids in length.

224. The DNA-targeting system of any of claims 152-220, wherein the transcriptional activation domain is at least at or about 70 amino acids in length.

225. The DNA-targeting system of any of claims 152-220, wherein the transcriptional activation domain is at or about 120, 110, 100, 90, 80, 70, 60, 50, or 40 amino acids or less in length.

226. The DNA-targeting system of any of claims 152-220 and 225, wherein the transcriptional activation domain is 70 amino acids or less in length.

227. The DNA-targeting system of any of claims 152-220 and 225, wherein the transcriptional activation domain is 60 amino acids or less in length.

228. The DNA-targeting system of any of claims 152-220 and 225, wherein the transcriptional activation domain is 50 amino acids or less in length.

229. The DNA-targeting system of any of claims 152-219, wherein the transcriptional activation domain is between at or about 40 and at or about 120, at or about 40 and at or about 110, at or about 40 and at or about 100, at or about 40 and at or about 90, at or about 40 and at or about 80, at or about 40 and at or about 70, at or about 40 and at or about 60, or at or about 40 and at or about 50 amino acids in length.

230. The DNA-targeting system of any of claims 152-229, wherein the fusion protein comprises a multipartite effector comprising at least two of the two or more transcriptional activation domains.

231. The DNA-targeting system claim 230, wherein the multipartite effector is composed of two transcriptional activation domains.

232. The DNA-targeting system claim 230 or claim 231, wherein the multipartite effector is set forth in any of SEQ ID NOS:140-153, a portion thereof, or an amino acid sequence that has at least 90%, 91%, 92%, 93%, 94%, 95%, 96%, 97%, 98%, or 99% sequence identity to any of the foregoing.

233. The DNA-targeting system of any of claims 152-230, wherein the two or more transcriptional activation domains is two transcriptional activation domains.

234. The DNA-targeting system of any of claims 152-233, wherein the fusion protein comprises:
a transcriptional activation domain of PYGO1 and a transcriptional activation domain of NCOA3;
a transcriptional activation domain of NOTCH2 and a transcriptional activation domain of NCOA3;
a transcriptional activation domain of NCOA3 and a transcriptional activation domain of NCOA3;
a transcriptional activation domain of HSH2D and a transcriptional activation domain of NCOA3;
a transcriptional activation domain of FOXO3 and a transcriptional activation domain of NCOA3;
a transcriptional activation domain of NCOA2 and a transcriptional activation domain of NCOA3;
a transcriptional activation domain of ENL and a transcriptional activation domain of NCOA3;
a transcriptional activation domain of PYGO1 and a transcriptional activation domain of FOXO3;
a transcriptional activation domain of NOTCH2 and a transcriptional activation domain of FOXO3;
a transcriptional activation domain of NCOA3 and a transcriptional activation domain of FOXO3;
a transcriptional activation domain of HSH2D and a transcriptional activation domain of FOXO3;
a transcriptional activation domain of FOXO3 and a transcriptional activation domain of FOXO3;
a transcriptional activation domain of NCOA2 and a transcriptional activation domain of FOXO3;
a transcriptional activation domain of ENL and a transcriptional activation domain of FOXO3;
a transcriptional activation domain of MYBA and a transcriptional activation domain of FOXO3; or
a transcriptional activation domain of SERTAD2 and a transcriptional activation domain of NCOA2.

235. The DNA-targeting system of any of claims 152-234, wherein the fusion protein comprises, in N-terminus to C-terminus order:
a transcriptional activation domain of PYGO1, a linker, and a transcriptional activation domain of NCOA3;
a transcriptional activation domain of NOTCH2, a linker, and a transcriptional activation domain of NCOA3;
a transcriptional activation domain of NCOA3, a linker, and a transcriptional activation domain of NCOA3;
a transcriptional activation domain of HSH2D, a linker, and a transcriptional activation domain of NCOA3;
a transcriptional activation domain of FOXO3, a linker, and a transcriptional activation domain of NCOA3;
a transcriptional activation domain of NCOA2, a linker, and a transcriptional activation domain of NCOA3;
a transcriptional activation domain of ENL, a linker, and a transcriptional activation domain of NCOA3;
a transcriptional activation domain of PYGO1, a linker, and a transcriptional activation domain of FOXO3;
a transcriptional activation domain of NOTCH2, a linker, and a transcriptional activation domain of FOXO3;
a transcriptional activation domain of NCOA3, a linker, and a transcriptional activation domain of FOXO3;
a transcriptional activation domain of HSH2D, a linker, and a transcriptional activation domain of FOXO3;
a transcriptional activation domain of FOXO3, a linker, and a transcriptional activation domain of FOXO3;
a transcriptional activation domain of NCOA2, a linker, and a transcriptional activation domain of FOXO3; or
a transcriptional activation domain of ENL, a linker, and a transcriptional activation domain of FOXO3.

236. The DNA-targeting system of any of claims 152-235, wherein the fusion protein comprises the sequence set forth in any one of SEQ ID NOS:140-153, or an amino acid sequence that has at least 90%, 91%, 92%, 93%, 94%, 95%, 96%, 97%, 98%, or 99% sequence identity to any of the foregoing.

237. The DNA-targeting system of any of claims 152-236, wherein the fusion protein comprises the sequence set forth in SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, or SEQ ID NO:153.

238. The DNA-targeting system of claim any of claims 152-237, wherein the fusion protein comprises, in N-terminus to C-terminus order:
a dCas, optionally a dCas9, a transcriptional activation domain of PYGO1 and a transcriptional activation domain of NCOA3;
a dCas, optionally a dCas9, a transcriptional activation domain of NOTCH2 and a transcriptional activation domain of NCOA3;
a dCas, optionally a dCas9, a transcriptional activation domain of NCOA3 and a transcriptional activation domain of NCOA3;
a dCas, optionally a dCas9, a transcriptional activation domain of HSH2D and a transcriptional activation domain of NCOA3;
a dCas, optionally a dCas9, a transcriptional activation domain of FOXO3 and a transcriptional activation domain of NCOA3;
a dCas, optionally a dCas9, a transcriptional activation domain of NCOA2 and a transcriptional activation domain of NCOA3;
a dCas, optionally a dCas9, a transcriptional activation domain of ENL and a transcriptional activation domain of NCOA3;
a dCas, optionally a dCas9, a transcriptional activation domain of PYGO1 and a transcriptional activation domain of FOXO3;
a dCas, optionally a dCas9, a transcriptional activation domain of NOTCH2 and a transcriptional activation domain of FOXO3;
a dCas, optionally a dCas9, a transcriptional activation domain of NCOA3 and a transcriptional activation domain of FOXO3;
a dCas, optionally a dCas9, a transcriptional activation domain of HSH2D and a transcriptional activation domain of FOXO3;
a dCas, optionally a dCas9, a transcriptional activation domain of FOXO3 and a transcriptional activation domain of FOXO3;
a dCas, optionally a dCas9, a transcriptional activation domain of NCOA2 and a transcriptional activation domain of FOXO3; or
a dCas, optionally a dCas9, a transcriptional activation domain of ENL and a transcriptional activation domain of FOXO3.

239. The DNA-targeting system of claim any of claims 152-237, wherein the fusion protein comprises, in N-terminus to C-terminus order:
a ZFP, a transcriptional activation domain of PYGO1 and a transcriptional activation domain of NCOA3;
a ZFP, a transcriptional activation domain of NOTCH2 and a transcriptional activation domain of NCOA3;
a ZFP, a transcriptional activation domain of NCOA3 and a transcriptional activation domain of NCOA3;
a ZFP, a transcriptional activation domain of HSH2D and a transcriptional activation domain of NCOA3;
a ZFP, a transcriptional activation domain of FOXO3 and a transcriptional activation domain of NCOA3;
a ZFP, a transcriptional activation domain of NCOA2 and a transcriptional activation domain of NCOA3;
a ZFP, a transcriptional activation domain of ENL and a transcriptional activation domain of NCOA3;
a ZFP, a transcriptional activation domain of PYGO1 and a transcriptional activation domain of FOXO3;
a ZFP, a transcriptional activation domain of NOTCH2 and a transcriptional activation domain of FOXO3;
a ZFP, a transcriptional activation domain of NCOA3 and a transcriptional activation domain of FOXO3;
a ZFP, a transcriptional activation domain of HSH2D and a transcriptional activation domain of FOXO3;
a ZFP, a transcriptional activation domain of FOXO3 and a transcriptional activation domain of FOXO3;
a ZFP, a transcriptional activation domain of NCOA2 and a transcriptional activation domain of FOXO3; or
a ZFP, a transcriptional activation domain of ENL and a transcriptional activation domain of FOXO3.

240. The DNA-targeting system of any of claims 152-237, wherein the fusion protein comprises, in N-terminus to C-terminus order:
a transcriptional activation domain of PYGO1, a transcriptional activation domain of NCOA3, and a dCas, optionally a dCas9;
a transcriptional activation domain of NOTCH2, a transcriptional activation domain of NCOA3, and a dCas, optionally a dCas9;
a transcriptional activation domain of NCOA3, a transcriptional activation domain of NCOA3, and a dCas, optionally a dCas9;
a transcriptional activation domain of HSH2D, a transcriptional activation domain of NCOA3, and a dCas, optionally a dCas9;
a transcriptional activation domain of FOXO3, a transcriptional activation domain of NCOA3, and a dCas, optionally a dCas9;
a transcriptional activation domain of NCOA2, a transcriptional activation domain of NCOA3, and a dCas, optionally a dCas9;
a transcriptional activation domain of ENL, a transcriptional activation domain of NCOA3, and a dCas, optionally a dCas9;
a transcriptional activation domain of PYGO1, a transcriptional activation domain of FOXO3, and a dCas, optionally a dCas9;
a transcriptional activation domain of NOTCH2, a transcriptional activation domain of FOXO3, and a dCas, optionally a dCas9;
a transcriptional activation domain of NCOA3, a transcriptional activation domain of FOXO3, and a dCas, optionally a dCas9;
a transcriptional activation domain of HSH2D, a transcriptional activation domain of FOXO3, and a dCas, optionally a dCas9;
a transcriptional activation domain of FOXO3, a transcriptional activation domain of FOXO3, and a dCas, optionally a dCas9;
a transcriptional activation domain of NCOA2, a transcriptional activation domain of FOXO3, and a dCas, optionally a dCas9; or
a transcriptional activation domain of ENL, a transcriptional activation domain of FOXO3, and a dCas, optionally a dCas9.

241. The DNA-targeting system of any of claims 152-237, wherein the fusion protein comprises, in N-terminus to C-terminus order:
a transcriptional activation domain of PYGO1, a transcriptional activation domain of NCOA3, and a ZFP;
a transcriptional activation domain of NOTCH2, a transcriptional activation domain of NCOA3, and a ZFP;
a transcriptional activation domain of NCOA3, a transcriptional activation domain of NCOA3, and a ZFP;
a transcriptional activation domain of HSH2D, a transcriptional activation domain of NCOA3, and a ZFP;
a transcriptional activation domain of FOXO3, a transcriptional activation domain of NCOA3, and a ZFP;
a transcriptional activation domain of NCOA2, a transcriptional activation domain of NCOA3, and a ZFP;
a transcriptional activation domain of ENL, a transcriptional activation domain of NCOA3, and a ZFP;
a transcriptional activation domain of PYGO1, a transcriptional activation domain of FOXO3, and a ZFP;
a transcriptional activation domain of NOTCH2, a transcriptional activation domain of FOXO3, and a ZFP;
a transcriptional activation domain of NCOA3, a transcriptional activation domain of FOXO3, and a ZFP;
a transcriptional activation domain of HSH2D, a transcriptional activation domain of FOXO3, and a ZFP;
a transcriptional activation domain of FOXO3, a transcriptional activation domain of FOXO3, and a ZFP;
a transcriptional activation domain of NCOA2, a transcriptional activation domain of FOXO3, and a ZFP; or
a transcriptional activation domain of ENL, a transcriptional activation domain of FOXO3, and a ZFP.

242. The DNA-targeting system of any of claims 152-230 and 234-241, wherein the two or more transcriptional activation domains is three transcriptional activation domains.

243. The DNA-targeting system of any of claims 152-230 and 242, wherein the fusion protein comprises a multipartite effector comprising at least three transcriptional activation domains.

244. The DNA-targeting system of claim 230 and 243, wherein the multipartite effector is composed of two transcriptional activation domains or three transcriptional activation domains.

245. The DNA-targeting system of claim 230, 243 and 244, wherein the multipartite effector is set forth in any of SEQ ID NOS:154-160 and 377, a portion thereof, or an amino acid sequence that has at least 90%, 91%, 92%, 93%, 94%, 95%, 96%, 97%, 98%, or 99% sequence identity to any of the foregoing.

246. The DNA-targeting system of any of claims 152-230 and 234-245, wherein the fusion protein comprises:
a transcriptional activation domain of PYGO1, a transcriptional activation domain of FOXO3, and a transcriptional activation domain of NCOA3;
a transcriptional activation domain of NOTCH2, a transcriptional activation domain of FOXO3, and a transcriptional activation domain of NCOA3;
a transcriptional activation domain of NCOA3, a transcriptional activation domain of FOXO3, and a transcriptional activation domain of NCOA3;
a transcriptional activation domain of HSH2D, a transcriptional activation domain of FOXO3, and a transcriptional activation domain of NCOA3;
a transcriptional activation domain of FOXO3, a transcriptional activation domain of FOXO3, and a transcriptional activation domain of NCOA3;
a transcriptional activation domain of NCOA2, a transcriptional activation domain of FOXO3, and a transcriptional activation domain of NCOA3;
a transcriptional activation domain of ENL, a transcriptional activation domain of FOXO3, and a transcriptional activation domain of NCOA3; or
a transcriptional activation domain of NCOA3, a transcriptional activation domain of FOXO3, and a transcriptional activation domain of FOXO3.

247. The DNA-targeting system of any of claims 230 and 243-246, wherein the multipartite effector comprises, in the N-terminal to C-terminal direction, domains from FOXO3, FOXO3, and NCOA3, respectively.

248. The DNA-targeting system of claim 247, wherein the multipartite effector comprises the sequence set forth in SEQ ID NO:158, or an amino acid sequence that has at least 90%, 91%, 92%, 93%, 94%, 95%, 96%, 97%, 98%, or 99% sequence identity thereto.

249. The DNA-targeting system of any of claims 230 and 243-246, wherein the multipartite effector comprises, in the N-terminal to C-terminal direction, domains from NCOA3, FOXO3, and NCOA3, respectively.

250. The DNA-targeting system of claim 249, wherein the multipartite effector comprises the sequence set forth in SEQ ID NO:156, or an amino acid sequence that has at least 90%, 91%, 92%, 93%, 94%, 95%, 96%, 97%, 98%, or 99% sequence identity thereto.

251. The DNA-targeting system of any of claims 230 and 243-246, wherein the multipartite effector comprises, in the N-terminal to C-terminal direction, domains from NCOA2, FOXO3, and NCOA3, respectively.

252. The DNA-targeting system of claim 251, wherein the multipartite effector comprises the sequence set forth in SEQ ID NO:159, or an amino acid sequence that has at least 90%, 91%, 92%, 93%, 94%, 95%, 96%, 97%, 98%, or 99% sequence identity thereto.

253. The DNA-targeting system of any of claims 230 and 243-246, wherein the multipartite effector comprises, in the N-terminal to C-terminal direction, domains from PYGO1, FOXO3, and NCOA3, respectively.

254. The DNA-targeting system of claim 253, wherein the multipartite effector comprises the sequence set forth in SEQ ID NO:154, or an amino acid sequence that has at least 90%, 91%, 92%, 93%, 94%, 95%, 96%, 97%, 98%, or 99% sequence identity thereto.

255. The DNA-targeting system of any of claims 230 and 243-246, wherein the multipartite effector comprises, in the N-terminal to C-terminal direction, domains from NCOA3, FOXO3, and FOXO3, respectively.

256. The DNA-targeting system of claim 255, wherein the multipartite effector comprises the sequence set forth in SEQ ID NO:377, or an amino acid sequence that has at least 90%, 91%, 92%, 93%, 94%, 95%, 96%, 97%, 98%, or 99% sequence identity thereto.

257. The DNA-targeting system of any one of any of claims 152-230 and 234-256, wherein the fusion protein comprises, in N-terminus to C-terminus order:
a transcriptional activation domain of PYGO1, a linker, a transcriptional activation domain of FOXO3, a linker, and a transcriptional activation domain of NCOA3;
a transcriptional activation domain of NOTCH2, a linker, a transcriptional activation domain of FOXO3, a linker, and a transcriptional activation domain of NCOA3;
a transcriptional activation domain of NCOA3, a linker, a transcriptional activation domain of FOXO3, a linker, and a transcriptional activation domain of NCOA3;
a transcriptional activation domain of HSH2D, a linker, a transcriptional activation domain of FOXO3, a linker, and a transcriptional activation domain of NCOA3;
a transcriptional activation domain of FOXO3, a linker, a transcriptional activation domain of FOXO3, a linker, and a transcriptional activation domain of NCOA3;
a transcriptional activation domain of NCOA2, a linker, a transcriptional activation domain of FOXO3, a linker, and a transcriptional activation domain of NCOA3;
a transcriptional activation domain of ENL, a linker, a transcriptional activation domain of FOXO3, a linker, and a transcriptional activation domain of NCOA3; or
a transcriptional activation domain of NCOA3, a linker, a transcriptional activation domain of FOXO3, a linker, and a transcriptional activation domain of FOXO3.

258. The DNA-targeting system of any of claims 152-230 and 234-257, wherein the fusion protein comprises the sequence set forth in any one of SEQ ID NOS:154-160 and 377 or an amino acid sequence that has at least 90%, 91%, 92%, 93%, 94%, 95%, 96%, 97%, 98%, or 99% sequence identity to any of the foregoing.

259. The DNA-targeting system of any of claims 152-230 and 234-258, wherein the fusion protein comprises the sequence set forth in SEQ ID NO:154, SEQ ID NO:155, SEQ ID NO:156, SEQ ID NO:157, SEQ ID NO:158, SEQ ID NO:159, or SEQ ID NO:160 or SEQ ID NO: 377.

260. The DNA-targeting system of any of claims 152-230 and 234-259, wherein the fusion protein comprises a transcriptional activation domain of PYGO1, a linker, a transcriptional activation domain of FOXO3, a linker, and a transcriptional activation domain of NCOA3.

261. The DNA-targeting system of any of claims 152-230 and 234-260, wherein the fusion protein comprises the sequence set forth in SEQ ID NO:154.

262. The DNA-targeting system of any of claims 152-230 and 234-259, wherein the fusion protein comprises a transcriptional activation domain of NCOA3, a linker, a transcriptional activation domain of FOXO3, a linker, and a transcriptional activation domain of NCOA3.

263. The DNA-targeting system of any of claims 152-230 and 234-259, and 262, wherein the fusion protein comprises the sequence set forth in SEQ ID NO:156.

264. The DNA-targeting system of any of claims 152-230 and 234-259, wherein the fusion protein comprises a transcriptional activation domain of FOXO3, a linker, a transcriptional activation domain of FOXO3, a linker, and a transcriptional activation domain of NCOA3.

265. The DNA-targeting system of any of claims 152-230 and 234-259, and 264, wherein the fusion protein comprises the sequence set forth in SEQ ID NO:158.

266. The DNA-targeting system of any of claims 152-230 and 234-259, wherein the fusion protein comprises a transcriptional activation domain of NCOA2, a linker, a transcriptional activation domain of FOXO3, a linker, and a transcriptional activation domain of NCOA3.

267. The DNA-targeting system of any of claims 152-230 and 234-259, and 266, wherein the fusion protein comprises the sequence set forth in SEQ ID NO:159.

268. The DNA-targeting system of any of claims 152-230 and 234-259, wherein the fusion protein comprises, in N-terminus to C-terminus order:
a Cas9, optionally dCas9, a linker, a transcriptional activation domain of PYGO1, a linker, a transcriptional activation domain of FOXO3, a linker, and a transcriptional activation domain of NCOA3;
a Cas9, optionally dCas9, a linker, a transcriptional activation domain of NOTCH2, a linker, a transcriptional activation domain of FOXO3, a linker, and a transcriptional activation domain of NCOA3;
a Cas9, optionally dCas9, a linker, a transcriptional activation domain of NCOA3, a linker, a transcriptional activation domain of FOXO3, a linker, and a transcriptional activation domain of NCOA3;
a Cas9, optionally dCas9, a linker, a transcriptional activation domain of HSH2D, a linker, a transcriptional activation domain of FOXO3, a linker, and a transcriptional activation domain of NCOA3;
a Cas9, optionally dCas9, a linker, a transcriptional activation domain of FOXO3, a linker, a transcriptional activation domain of FOXO3, a linker, and a transcriptional activation domain of NCOA3;
a Cas9, optionally dCas9, a linker, a transcriptional activation domain of NCOA2, a linker, a transcriptional activation domain of FOXO3, a linker, and a transcriptional activation domain of NCOA3; or
a Cas9, optionally dCas9, a linker, a transcriptional activation domain of ENL, a linker, a transcriptional activation domain of FOXO3, a linker, and a transcriptional activation domain of NCOA3; or
a Cas9, optionally dCas9, a linker, a transcriptional activation domain of NCOA3, a linker, a transcriptional activation domain of FOXO3, a linker, and a transcriptional activation domain of FOXO3.

269. The DNA-targeting system of any of claims 152-230 and 234-259, wherein the fusion protein comprises, in N-terminus to C-terminus order:
a ZFP, a linker, a transcriptional activation domain of PYGO1, a linker, a transcriptional activation domain of FOXO3, a linker, and a transcriptional activation domain of NCOA3;
a ZFP, a linker, a transcriptional activation domain of NOTCH2, a linker, a transcriptional activation domain of FOXO3, a linker, and a transcriptional activation domain of NCOA3;
a ZFP, a linker, a transcriptional activation domain of NCOA3, a linker, a transcriptional activation domain of FOXO3, a linker, and a transcriptional activation domain of NCOA3;
a ZFP, a linker, a transcriptional activation domain of HSH2D, a linker, a transcriptional activation domain of FOXO3, a linker, and a transcriptional activation domain of NCOA3;
a ZFP, a linker, a transcriptional activation domain of FOXO3, a linker, a transcriptional activation domain of FOXO3, a linker, and a transcriptional activation domain of NCOA3;
a ZFP, a linker, a transcriptional activation domain of NCOA2, a linker, a transcriptional activation domain of FOXO3, a linker, and a transcriptional activation domain of NCOA3; or
a ZFP, a linker, a transcriptional activation domain of ENL, a linker, a transcriptional activation domain of FOXO3, a linker, and a transcriptional activation domain of NCOA3; or
a ZFP, a linker, a transcriptional activation domain of NCOA3, a linker, a transcriptional activation domain of FOXO3, a linker, and a transcriptional activation domain of FOXO3.

270. The DNA-targeting system of any of claims 152-230 and 234-259, wherein the fusion protein comprises, in N-terminus to C-terminus order:
a transcriptional activation domain of PYGO1, a linker, a transcriptional activation domain of FOXO3, a linker, a transcriptional activation domain of NCOA3, a linker, and a Cas9, optionally a dCas9;
a transcriptional activation domain of NOTCH2, a linker, a transcriptional activation domain of FOXO3, a linker, a transcriptional activation domain of NCOA3, a linker, and a Cas9, optionally a dCas9;
a transcriptional activation domain of NCOA3, a linker, a transcriptional activation domain of FOXO3, a linker, a transcriptional activation domain of NCOA3, a linker, and a Cas9, optionally a dCas9;
a transcriptional activation domain of HSH2D, a linker, a transcriptional activation domain of FOXO3, a linker, a transcriptional activation domain of NCOA3, a linker, and a Cas9, optionally a dCas9;
a transcriptional activation domain of FOXO3, a linker, a transcriptional activation domain of FOXO3, a linker, a transcriptional activation domain of NCOA3, a linker, and a Cas9, optionally a dCas9;
a transcriptional activation domain of NCOA2, a linker, a transcriptional activation domain of FOXO3, a linker, a transcriptional activation domain of NCOA3, a linker, and a Cas9, optionally a dCas9;
a transcriptional activation domain of ENL, a linker, a transcriptional activation domain of FOXO3, a linker, a transcriptional activation domain of NCOA3, a linker, and a Cas9, optionally a dCas9; or
a transcriptional activation domain of NCOA3, a linker, a transcriptional activation domain of FOXO3, a linker, a transcriptional activation domain of FOXO3, a linker, and a Cas9, optionally a dCas9.

271. The DNA-targeting system of any of claims 152-230 and 234-259, wherein the fusion protein comprises, in N-terminus to C-terminus order:
a transcriptional activation domain of PYGO1, a linker, a transcriptional activation domain of FOXO3, a linker, a transcriptional activation domain of NCOA3, a linker, and a ZFP;
a transcriptional activation domain of NOTCH2, a linker, a transcriptional activation domain of FOXO3, a linker, a transcriptional activation domain of NCOA3, a linker, and a ZFP;
a transcriptional activation domain of NCOA3, a linker, a transcriptional activation domain of FOXO3, a linker, a transcriptional activation domain of NCOA3, a linker, and a ZFP;
a transcriptional activation domain of HSH2D, a linker, a transcriptional activation domain of FOXO3, a linker, a transcriptional activation domain of NCOA3, a linker, and a ZFP;
a transcriptional activation domain of FOXO3, a linker, a transcriptional activation domain of FOXO3, a linker, a transcriptional activation domain of NCOA3, a linker, and a ZFP;
a transcriptional activation domain of NCOA2, a linker, a transcriptional activation domain of FOXO3, a linker, a transcriptional activation domain of NCOA3, a linker, and a ZFP;
a transcriptional activation domain of ENL, a linker, a transcriptional activation domain of FOXO3, a linker, a transcriptional activation domain of NCOA3, a linker, and a ZFP; or
a transcriptional activation domain of NCOA3, a linker, a transcriptional activation domain of FOXO3, a linker, a transcriptional activation domain of FOXO3, a linker, and a ZFP.

272. The DNA-targeting system of any of claims 152-230, 234-241, and 246-271, wherein the two or more transcriptional activation domains comprises four transcriptional activation domains.

273. The DNA-targeting system of any of claims 152-230, 234-241, and 246-271, wherein the two or more transcriptional activation domains comprises five transcriptional activation domains.

274. The DNA-targeting system of any of claims 152-273, further comprising one or more linkers.

275. The DNA-targeting system of any of claims 152-274, wherein a linker of one or more linkers is positioned between the two or more transcriptional activation domains and/or positioned between the polypeptide component of the DNA-targeting domain and one of the two or more transcriptional activation domains.

276. The DNA-targeting system of claim 274 or 275, wherein the linker is a polypeptide linker.

277. The DNA-targeting system of claim 276, wherein the polypeptide linker comprises a sequence selected from among SEQ ID NOS:62-67, 96, and 137-139.

278. The DNA-targeting system of any of claims 152-277, further comprising one or more nuclear localization signals (NLSs).

279. The DNA-targeting system of claim 278, wherein the one or more NLSs comprises two or more NLSs.

280. The DNA-targeting system of claim 278 or 279, wherein a NLS of one or more NLSs is positioned between the two or more transcriptional activation domains.

281. The DNA-targeting system of any of claims 236-280, wherein a NLS of the one or more NLSs is positioned between the polypeptide component of the DNA-targeting domain and one of the two or more transcriptional activation domains.

282. The DNA-targeting system of any of claims 236-281, wherein the one or more NLSs comprises a sequence selected from among SEQ ID NOS:69-84.

283. The DNA-targeting system of any of claims 152-282, wherein the fusion protein comprises the sequence set forth in any one of SEQ ID NOS:181-187, or an amino acid sequence that has at least 90%, 91%, 92%, 93%, 94%, 95%, 96%, 97%, 98%, or 99% sequence identity to any of the foregoing.

284. The DNA-targeting system of any of claims 152-283, wherein the fusion protein comprises the sequence set forth in SEQ ID NO:181, SEQ ID NO: 182, SEQ ID NO:183, SEQ ID NO:184, SEQ ID NO:185, SEQ ID NO:186, or SEQ ID NO: 187.

285. The DNA-targeting system of any of claims 152-284, further comprising a tag.

286. The DNA-targeting system of claim 285, wherein the tag comprises an epitope tag or a split protein tag.

287. The DNA-targeting system of claim 285 or 286, wherein the tag is selected from among SEQ ID NOS:61, 88, 92, and 167.

288. The DNA-targeting system of any of claims 152-287, wherein the fusion protein comprises the sequence set forth in any one of SEQ ID NOS:272-278, or an amino acid sequence that has at least 90%, 91%, 92%, 93%, 94%, 95%, 96%, 97%, 98%, or 99% sequence identity to any of the foregoing.

289. The DNA-targeting system of any of claims 152-288, wherein the fusion protein comprises the sequence set forth in SEQ ID NO:272, SEQ ID NO:273, SEQ ID NO:274, SEQ ID NO:275, SEQ ID NO:276, SEQ ID NO:277, or SEQ ID NO:278.

290. The DNA-targeting system of any of claims 152-289, wherein the DNA-targeting domain is capable of hybridizing to the target site or is complementary to the target site at the endogenous locus.

291. The DNA-targeting system of any of claims 152-290, wherein the at least one gRNA is capable of hybridizing to the target site or is complementary to the target site at the endogenous locus.

292. The DNA-targeting system of any of claims 152-291, wherein the target site is located at a regulatory DNA element of the endogenous locus.

293. The DNA-targeting system of claim 292, wherein the regulatory DNA element is selected from among a promoter, an upstream regulatory element, an enhancer, an exon, an intron, a 5′ untranslated region (UTR), a 3′ UTR, or a downstream regulatory element.

294. The DNA-targeting system of any of claims 152-293, wherein the endogenous locus is in a human cell.

295. The DNA-targeting system of any of claims 152-294, wherein the endogenous locus is in a stem cell; liver cell, optionally a hepatocyte; muscle cell; heart cell, optionally a cardiomyocyte; brain cell, optionally a neuron; blood cell; immune cell, optionally a lymphoid cell, optionally a T cell; or a cell derived from any of the foregoing.

296. The DNA-targeting system of any of claims 152-295, wherein the endogenous locus is FXN.

297. The DNA-targeting system of any of claims 152-296, wherein the target site is located within the genomic coordinates hg38 chr9:69,027,282-69,028,497 or hg38 chr9:69,027,282-69,028,497.

298. The DNA-targeting system of any of claims 152-297, wherein the target site is located within the genomic coordinates hg38 chr9:69,027,615-69,028,101.

299. The DNA-targeting system of any of claims 152-297, wherein the target site comprises a sequence set forth in any one of SEQ ID NOS:208-228.

300. The DNA-targeting system of any of claims 152-297 and 299, wherein the target site comprises a sequence set forth in SEQ ID NO:208.

301. The DNA-targeting system of any of claims 152-297 and 299, wherein the target site comprises a sequence set forth in SEQ ID NO:214.

302. The DNA-targeting system of any of claims 152-297, wherein the target site comprises a sequence set forth in SEQ ID NO:228.

303. The DNA-targeting system of any of claims 151, 153, 155, and 158-297, wherein the gRNA comprises a sequence set forth in any one of SEQ ID NOS:229-249.

304. The DNA-targeting system of any of claims 151, 153, 155, 158-297, and 303, wherein the gRNA comprises a sequence set forth in SEQ ID NO:229.

305. The DNA-targeting system of any of claims 151, 153, 155, 158-297, and 303, wherein the gRNA comprises a sequence set forth in SEQ ID NO:235.

306. The DNA-targeting system of any of claims 151, 153, 155, 158-297, and 303, wherein the gRNA comprises a sequence set forth in SEQ ID NO:249.

307. A polynucleotide comprising a sequence encoding the fusion protein of any of claims 1-149 or the DNA-targeting system of any of claims 150-306, or a portion or a component of any of the foregoing.

308. The polynucleotide of claim 307, wherein the sequence encoding the fusion protein comprises a sequence set forth in any one of SEQ ID NOS:109-122, or a nucleic acid sequence that has at least 90%, 91%, 92%, 93%, 94%, 95%, 96%, 97%, 98%, or 99% sequence identity to any of the foregoing.

309. The polynucleotide of claim 307 or 308, wherein the sequence encoding the fusion protein comprises a sequence set forth in any one of SEQ ID NOS:109-122.

310. The polynucleotide of claim 307, wherein the sequence encoding the fusion protein comprises a sequence set forth in any one of SEQ ID NOS:123-129, or a nucleic acid sequence that has at least 90%, 91%, 92%, 93%, 94%, 95%, 96%, 97%, 98%, or 99% sequence identity to any of the foregoing.

311. The polynucleotide of claim 307 or 310, wherein the sequence encoding the fusion protein comprises a sequence set forth in any one of SEQ ID NOS:123-129.

312. A plurality of polynucleotides, comprising a first polynucleotide comprising the polynucleotide of any of claims 307-311, and one or more second polynucleotides encoding an additional portion or an additional component of the fusion protein of any of claims 1-149 or the DNA-targeting system of any of claims 150-306, or a portion or a component of any of the foregoing.

313. A vector comprising the polynucleotide of any of claims 307-311.

314. A vector comprising the plurality of polynucleotides of claim 312.

315. The vector of claim 313 or 314, wherein the vector is a viral vector.

316. The vector of claim 315, wherein the viral vector is an AAV vector.

317. The vector of claim 316, wherein the AAV vector is selected from among an AAV1, AAV2, AAV3, AAV4, AAV5, AAV6, AAV7, AAV8, AAV9, AAV10, AAV11, AAV12, or AAV-DJ vector.

318. The vector of any of claims 315-317, wherein the viral vector is an AAV9 vector.

319. The vector of claim 313 or 314, wherein the vector is a non-viral vector selected from: a lipid nanoparticle, a liposome, an exosome, or a cell penetrating peptide.

320. A plurality of vectors, comprising a first vector comprising the vector of any of claims 313-319, and one or more second vectors comprising the one or more second polynucleotide of the plurality of polynucleotides of claim 312.

321. A cell comprising the fusion protein of any of claims 1-149, the DNA-targeting system of any of claims 150-306, the polynucleotide of any of claims 307-311, the plurality of polynucleotides of claim 312, the vector of any of claims 313-319, or the plurality of vectors of claim 320, or a portion or a component of any of the foregoing.

322. A method for modulating the expression of an endogenous locus in a cell, the method comprising introducing the fusion protein of any of claims 1-149, the DNA-targeting system of any of claims 150-306, the polynucleotide of any of claims 307-311, the plurality of polynucleotides of claim 312, the vector of any of claims 313-319, or the plurality of vectors of claim 320, or a portion or a component of any of the foregoing, into the cell.

323. A method for modulating the expression of an endogenous locus in a subject, the method comprising administering the fusion protein of any of claims 1-149, the DNA-targeting system of any of claims 150-306, the polynucleotide of any of claims 307-311, the plurality of polynucleotides of claim 312, the vector of any of claims 313-319, or the plurality of vectors of claim 320, or a portion or a component of any of the foregoing, to the subject.

324. The method of claim 322 or 323, wherein the fusion protein or the DNA-targeting system increases transcription of an endogenous locus when recruited to a target site at the endogenous locus.

325. The method of claim 324, wherein the endogenous locus is in a human cell.

326. The method of claim 324 or 325, wherein the endogenous locus is in a stem cell; liver cell, optionally a hepatocyte; muscle cell; heart cell, optionally a cardiomyocyte; brain cell, optionally a neuron; blood cell; immune cell, optionally a lymphoid cell, optionally a T cell; or a cell derived from any of the foregoing.

327. The method of any of claims 322-326, wherein the endogenous locus is FXN.

328. The method of any of claims 322-327, wherein the target site is located within the genomic coordinates hg38 chr9:68,940,179-69,205,519 or hg38 chr9:69,027,282-69,028,497.

329. The method of any of claims 322-328, wherein the target site is located within the genomic coordinates hg38 chr9:69,027,615-69,028,101.

330. The method of any of claims 322-328, wherein the target site comprises a sequence set forth in any one of SEQ ID NOS:208-228.

331. The method of any of claims 322-328 and 330, wherein the target site comprises a sequence set forth in SEQ ID NO:208.

332. The method of any of claims 322-328 and 330, wherein the target site comprises a sequence set forth in SEQ ID NO:214.

333. The method of any of claims 322-330, wherein the target site comprises a sequence set forth in SEQ ID NO:228.

334. The method of any of claims 322-333, wherein the cell is from a subject that has or is suspected of having a disease or disorder or the subject has or is suspected of having a disease or disorder.

335. The method of claim 334, wherein the disease or disorder is associated with the reduction of expression of the endogenous locus.

336. The method of any of claims 322-335, wherein the introducing, contacting or administering is carried out in vivo or ex vivo.

337. The method of any of claims 322-336, wherein the subject is a human.

338. A pharmaceutical composition comprising the fusion protein of any of claims 1-149, the DNA-targeting system of any of claims 150-306, the polynucleotide of any of claims 307-311, the plurality of polynucleotides of claim 312, the vector of any of claims 313-319, or the plurality of vectors of claim 320, or a portion or a component of any of the foregoing.

339. The pharmaceutical composition of claim 338, for use in treating a disease or disorder.

340. The pharmaceutical composition of claim 338, for use in the manufacture of a medicament for treating a disease or disorder.

341. Use of the pharmaceutical composition of claim 338 for treating a disease or disorder.

342. Use of the pharmaceutical composition of claim 338 in the manufacture of a medicament for treating a disease or disorder.

343. The pharmaceutical composition for use or the use of any of claims 338-342, wherein the disease or disorder is associated with the reduction of expression of an endogenous locus.

344. The pharmaceutical composition for use or the use of any of claims 338-343, wherein the pharmaceutical composition is to be administered to a subject.

345. The pharmaceutical composition for use or the use of any of claims 338-344, wherein the administration is carried out in vivo or ex vivo.

346. The pharmaceutical composition for use or the use of any of claims 338-345, wherein the fusion protein or the DNA-targeting system increases transcription of an endogenous locus when recruited to a target site at the endogenous locus.

347. The pharmaceutical composition for use or the use of claim 346, wherein the endogenous locus is in a human cell.

348. The pharmaceutical composition for use or the use of claim 346 or 347, wherein the endogenous locus is in a stem cell; liver cell, optionally a hepatocyte; muscle cell; heart cell, optionally a cardiomyocyte; brain cell, optionally a neuron; blood cell; immune cell, optionally a lymphoid cell, optionally a T cell; or a cell derived from any of the foregoing.

349. The pharmaceutical composition for use or the use of any of claims 338-348, wherein the endogenous locus is frataxin (FXN).

350. The pharmaceutical composition for use or the use of any of claims 338-349, wherein the target site is located within the genomic coordinates hg38 chr9:68,940,179-69,205,519 or hg38 chr9:69,027,282-69,028,497.

351. The pharmaceutical composition for use or the use of any of claims 338-350, wherein the target site is located within the genomic coordinates hg38 chr9:69,027,615-69,028,101.

352. The pharmaceutical composition for use or the use of any of claims 338-350, wherein the target site comprises a sequence set forth in any one of SEQ ID NOS:208-228.

353. The pharmaceutical composition for use or the use of any of claims 338-350 and 352, wherein the target site comprises a sequence set forth in SEQ ID NO:208.

354. The pharmaceutical composition for use or the use of any of claims 338-350 and 352, wherein the target site comprises a sequence set forth in SEQ ID NO:214.

355. The pharmaceutical composition for use or the use of any of claims 338-352, wherein the target site comprises a sequence set forth in SEQ ID NO:228.

356. The pharmaceutical composition for use or the use of any of claims 338-355, wherein the subject is a human.


SEQUENCE LISTING (395 sequences)

SEQ ID NO: 1 - DNA, 3156 bp, synthetic construct
  misc_feature 1..3156: note = dSaCas9 (nt)
  source 1..3156: mol_type = other DNA
aagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 2 - protein, 1052 aa, synthetic construct
  REGION 1..1052: note = dSaCas9 (aa)
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 1053 aa, synthetic construct
  REGION 1..1053: note = WT Sa Cas9 (aa)
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 1052 aa, synthetic construct
  REGION 1..1052: note = WT Sa Cas9 (aa)
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 5 - DNA, 4101 bp, synthetic construct
  misc_feature 1..4101: note = dSpCas9 (nt)
  source 1..4101: mol_type = other DNA
gacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 6 - protein, 1367 aa, synthetic construct
  REGION 1..1367: note = dSpCas9 (aa)
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 1368 aa, synthetic construct [sequence omitted]

SEQ ID NO: 8 - protein, 1367 aa, synthetic construct
  REGION 1..1367: note = WT Sp Cas9 (aa)
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 9 - DNA, 240 bp, synthetic construct
  misc_feature 1..240: note = DPOLA (nt)
  source 1..240: mol_type = other DNA
aagagcaagaagggcagacaggaggccctggagagactgaagaaggccaaggccggcgagaagtacaagtacgaggtggaggacttcaccggcgtgtacgaggaggtggacgaggagcagtacagcaagctggtgcaggccagacaggacgacgactggatcgtggacgacgacggcatcggctacgtggaggacggcagagagatcttcgacgacgacctggaggacgacgccctggac

SEQ ID NO: 10 - DNA, 240 bp, synthetic construct
  misc_feature 1..240: note = ENL (nt)
  source 1..240: mol_type = other DNA
agcaagcccgagaagatcctgaagaagggcacctacgacaaggcctacaccgacgagctggtggagctgcacagaagactgatggccctgagagagagaaacgtgctgcagcagatcgtgaacctgatcgaggagaccggccacttcaacgtgaccaacaccaccttcgacttcgacctgttcagcctggacgagaccaccgtgagaaagctgcagagctgcctggaggccgtggccacc

SEQ ID NO: 11 - DNA, 240 bp, synthetic construct
  misc_feature 1..240: note = FOXO3 (nt)
  source 1..240: mol_type = other DNA
gacagcctgagcggcagcagcctgtacagcaccagcgccaacctgcccgtgatgggccacgagaagttccccagcgacctggacctggacatgttcaacggcagcctggagtgcgacatggagagcatcatcagaagcgagctgatggacgccgacggcctggacttcaacttcgacagcctgatcagcacccagaacgtggtgggcctgaacgtgggcaacttcaccggcgccaagcag

SEQ ID NO: 12 - DNA, 240 bp, synthetic construct
  misc_feature 1..240: note = HSH2D (nt)
  source 1..240: mol_type = other DNA
cccgagtggttccacggcgccatcagcagagaggacgccgagaacctgctggagagccagcccctgggcagcttcctgatcagagtgagccacagccacgtgggctacaccctgagctacaaggcccagagcagctgctgccacttcatggtgaagctgctggacgacggcaccttcatgatccccggcgagaaggtggcccacaccagcctggacgccctggtgaccttccaccagcag

SEQ ID NO: 13 - DNA, 240 bp, synthetic construct
  misc_feature 1..240: note = NCOA2 (nt)
  source 1..240: mol_type = other DNA
cccttcggcagcagccccgacgacctgctgtgcccccaccccgctgctgaaagccccagcgacgagggcgctctgctggaccagctgtacctggccctgagaaacttcgacggcctggaggagatcgacagagccctgggcatccccgagctggtgagccagagccaggccgtggaccccgagcagttcagcagccaggacagcaacatcatgctggagcagaaggcccccgtgttcccc

SEQ ID NO: 14 - DNA, 240 bp, synthetic construct
  misc_feature 1..240: note = NCOA3 (nt)
  source 1..240: mol_type = other DNA
ctgagaaacagcctggacgacctggtgggcccccccagcaacctggagggccagagcgacgagagagccctgctggaccagctgcacaccctgctgagcaacaccgacgccaccggcctggaggagatcgacagagccctgggcatccccgagctggtgaaccagggccaggccctggagcccaagcaggacgccttccagggccaggaggctgccgtgatgatggaccagaaggccggc

SEQ ID NO: 15 - DNA, 240 bp, synthetic construct
  misc_feature 1..240: note = PSA1 (nt)
  source 1..240: mol_type = other DNA
ttcagaaaccagtacgacaacgacgtgaccgtgtggagcccccagggcagaatccaccagatcgagtacgccatggaggccgtgaagcagggcagcgccaccgtgggcctgaagagcaagacccacgccgtgctggtggccctgaagagagcccagagcgagctggccgcccaccagaagaagatcctgcacgtggacaaccacatcggcatcagcatcgccggcctgaccgccgacgcc

SEQ ID NO: 16 - DNA, 240 bp, synthetic construct
  misc_feature 1..240: note = PYGO1 (nt)
  source 1..240: mol_type = other DNA
agacacggccacagcagcagcgaccccgtgtacccctgcggcatctgcaccaacgaggtgaacgacgaccaggacgccatcctgtgcgaggccagctgccagaagtggttccacagaatctgcaccggcatgaccgagaccgcctacggcctgctgaccgccgaggccagcgctgtgtggggatgcgacacctgcatggccgacaaggacgtgcagctgatgagaaccagagagaccttc

SEQ ID NO: 17 - DNA, 240 bp, synthetic construct
  misc_feature 1..240: note = RBM39 (nt)
  source 1..240: mol_type = other DNA
ggccccatgagactgtacgtgggcagcctgcacttcaacatcaccgaggacatgctgagaggcatcttcgagcccttcggcagaatcgagagcatccagctgatgatggacagcgagaccggcagaagcaagggctacggcttcatcaccttcagcgacagcgagtgcgccaagaaggccctggagcagctgaacggcttcgagctggccggcagacccatgaaggtgggccacgtgacc

SEQ ID NO: 18 - DNA, 240 bp, synthetic construct
  misc_feature 1..240: note = HERC2 (nt)
  source 1..240: mol_type = other DNA
accctgatcagaaaggccgacctggagaaccacaacaaggacggcggcttctggaccgtgatcgacggcaaggtgtacgacatcaaggacttccagacccagagcctgaccggcaacagcatcctggcccagttcgccggcgaggaccccgtggtggccctggaagctgctctgcagttcgaggacaccagagagagcatgcacgccttctgcgtgggccagtacctggagcccgaccag

SEQ ID NO: 19 - DNA, 240 bp, synthetic construct
  misc_feature 1..240: note = DMD control (nt)
  source 1..240: mol_type = other DNA
ccctactacatcaaccacgagacccagaccacctgctgggaccaccccaagatgaccgagctgtaccagagcctggccgacctgaacaacgtgagattcagcgcctacaggaccgccatgaagctgagaagactgcagaaggccctgtgcctggacctgctgagcctgagcgccgcctgcgacgccttggaccagcacaacctgaagcagaacgaccagcccatggacatcctgcagatc

SEQ ID NO: 20 - DNA, 240 bp, synthetic construct
  misc_feature 1..240: note = NOTCH2 (nt)
  source 1..240: mol_type = other DNA
aacagatacagctgcgtgtgcagccccggcttcaccggccagagatgcaacatcgacatcgacgagtgcgccagcaacccctgcagaaagggcgccacctgcatcaacggcgtgaacggcttcagatgcatctgccccgagggcccccaccaccccagctgctacagccaggtgaacgagtgcctgagcaacccctgcatccacggcaactgcaccggcggcctgagcggctacaagtgc

SEQ ID NO: 21 - protein, 80 aa, synthetic construct
  REGION 1..80: note = Control peptide (aa)
GIVTLPHRYVYLIRFLADHVDASQSLTTRTRIDTFMCVACSFDGSNYTAVLSPQILAIRRNSCVRDMRFVARREYTKAVF

SEQ ID NO: 22 - protein, 80 aa, synthetic construct
  REGION 1..80: note = DPOLA (aa)
KSKKGRQEALERLKKAKAGEKYKYEVEDFTGVYEEVDEEQYSKLVQARQDDDWIVDDDGIGYVEDGREIFDDDLEDDALD

SEQ ID NO: 23 - protein, 80 aa, synthetic construct
  REGION 1..80: note = ENL (aa)
SKPEKILKKGTYDKAYTDELVELHRRLMALRERNVLQQIVNLIEETGHFNVTNTTFDFDLFSLDETTVRKLQSCLEAVAT

SEQ ID NO: 24 - protein, 80 aa, synthetic construct
  REGION 1..80: note = FOXO3 (aa)
DSLSGSSLYSTSANLPVMGHEKFPSDLDLDMFNGSLECDMESIIRSELMDADGLDFNFDSLISTQNVVGLNVGNFTGAKQ

SEQ ID NO: 25 - protein, 80 aa, synthetic construct
  REGION 1..80: note = HSH2D (aa)
PEWFHGAISREDAENLLESQPLGSFLIRVSHSHVGYTLSYKAQSSCCHFMVKLLDDGTFMIPGEKVAHTSLDALVTFHQQ

SEQ ID NO: 26 - protein, 80 aa, synthetic construct
  REGION 1..80: note = NCOA2 (aa)
PFGSSPDDLLCPHPAAESPSDEGALLDQLYLALRNFDGLEEIDRALGIPELVSQSQAVDPEQFSSQDSNIMLEQKAPVFP

SEQ ID NO: 27 - protein, 80 aa, synthetic construct
  REGION 1..80: note = NCOA3 (aa)
LRNSLDDLVGPPSNLEGQSDERALLDQLHTLLSNTDATGLEEIDRALGIPELVNQGQALEPKQDAFQGQEAAVMMDQKAG

SEQ ID NO: 28 - protein, 80 aa, synthetic construct
  REGION 1..80: note = PSA1 (aa)
FRNQYDNDVTVWSPQGRIHQIEYAMEAVKQGSATVGLKSKTHAVLVALKRAQSELAAHQKKILHVDNHIGISIAGLTADA

SEQ ID NO: 29 - protein, 80 aa, synthetic construct
  REGION 1..80: note = PYGO1 (aa)
RHGHSSSDPVYPCGICTNEVNDDQDAILCEASCQKWFHRICTGMTETAYGLLTAEASAVWGCDTCMADKDVQLMRTRETF

SEQ ID NO: 30 - protein, 80 aa, synthetic construct
  REGION 1..80: note = RBM39 (aa)
GPMRLYVGSLHFNITEDMLRGIFEPFGRIESIQLMMDSETGRSKGYGFITFSDSECAKKALEQLNGFELAGRPMKVGHVT

SEQ ID NO: 31 - protein, 80 aa, synthetic construct
  REGION 1..80: note = HERC2 (aa)
TLIRKADLENHNKDGGFWTVIDGKVYDIKDFQTQSLTGNSILAQFAGEDPVVALEAALQFEDTRESMHAFCVGQYLEPDQ

SEQ ID NO: 32 - protein, 80 aa, synthetic construct
  REGION 1..80: note = DMD (aa)
PYYINHETQTTCWDHPKMTELYQSLADLNNVRFSAYRTAMKLRRLQKALCLDLLSLSAACDALDQHNLKQNDQPMDILQI

SEQ ID NO: 33 - protein, 80 aa, synthetic construct
  REGION 1..80: note = NOTCH2 (aa)
NRYSCVCSPGFTGQRCNIDIDECASNPCRKGATCINGVNGFRCICPEGPHHPSCYSQVNECLSNPCIHGNCTGGLSGYKC

SEQ ID NO: 34 - protein, 617 aa, synthetic construct
  REGION 1..617: note = p300 core (aa)
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 35 - protein, 1468 aa, Homo sapiens
  REGION 1..1468: note = DNA polymerase alpha catalytic subunit (DPOLA) isoform 1 [Homo sapiens] (aa)
MAPVHGDDCEIGASALSDSGSFVSSRARREKKSKKGRQEALERLKKAKAGEKYKYEVEDFTGVYEEVDEEQYSKLVQARQDDDWIVDDDGIGYVEDGREIFDDDLEDDALDADEKGKDGKARNKDKRNVKKLAVTKPNNIKSMFIACAGKKTADKAVDLSKDGLLGDILQDLNTETPQITPPPVMILKKKRSIGASPNPFSVHTATAVPSGKIASPVSRKEPPLTPVPLKRAEFAGDDVQVESTEEEQESGAMEFEDGDFDEPMEVEEVDLEPMAAKAWDKESEPAEEVKQEADSGKGTVSYLGSFLPDVSCWDIDQEGDSSFSVQEVQVDSSHLPLVKGADEEQVFHFYWLDAYEDQYNQPGVVFLFGKVWIESAETHVSCCVMVKNIERTLYFLPREMKIDLNTGKETGTPISMKDVYEEFDEKIATKYKIMKFKSKPVEKNYAFEIPDVPEKSEYLEVKYSAEMPQLPQDLKGETFSHVFGTNTSSLELFLMNRKIKGPCWLEVKSPQLLNQPVSWCKVEAMALKPDLVNVIKDVSPPPLVVMAFSMKTMQNAKNHQNEIIAMAALVHHSFALDKAAPKPPFQSHFCVVSKPKDCIFPYAFKEVIEKKNVKVEVAATERTLLGFFLAKVHKIDPDIIVGHNIYGFELEVLLQRINVCKAPHWSKIGRLKRSNMPKLGGRSGFGERNATCGRMICDVEISAKELIRCKSYHLSELVQQILKTERVVIPMENIQNMYSESSQLLYLLEHTWKDAKFILQIMCELNVLPLALQITNIAGNIMSRTLMGGRSERNEFLLLHAFYENNYIVPDKQIFRKPQQKLGDEDEEIDGDTNKYKKGRKKAAYAGGLVLDPKVGFYDKFILLLDFNSLYPSIIQEFNICFTTVQRVASEAQKVTEDGEQEQIPELPDPSLEMGILPREIRKLVERRKQVKQLMKQQDLNPDLILQYDIRQKALKLTANSMYGCLGFSYSRFYAKPLAALVTYKGREILMHTKEMVQKMNLEVIYGDTDSIMINTNSTNLEEVFKLGNKVKSEVNKLYKLLEIDIDGVFKSLLLLKKKKYAALVVEPTSDGNYVTKQELKGLDIVRRDWCDLAKDTGNFVIGQILSDQSRDTIVENIQKRLIEIGENVLNGSVPVSQFEINKALTKDPQDYPDKKSLPHVHVALWINSQGGRKVKAGDTVSYVICQDGSNLTASQRAYAPEQLQKQDNLTIDTQYYLAQQIHPVVARICEPIDGIDAVLIATWLGLDPTQFRVHHYHKDEENDALLGGPAQLTDEEKYRDCERFKCPCPTCGTENIYDNVFDGSGTDMEPSLYRCSNIDCKASPLTFTVQLSNKLIMDIRRFIKKYYDGWLICEEPTCRNRTRHLPLQFSRTGPLCPACMKATLQPEYSDKSLYTQLCFYRYIFDAECALEKLTTDHEKDKLKKQFFTPKVLQDYRKLKNTAEQFLSRSGYSEVNLSKLFAGCAVKS

SEQ ID NO: 36 - protein, 600 aa, Homo sapiens
  REGION 1..600: note = protein ENL (ENL) isoform X1 [Homo sapiens] (aa)
MCAFPAFPQFWDSEIPGQRHQLRSSFCVCVCTCAPVCRLDSDFKRCTVQVRLELGHRAQLRKKPTTEGFTHDWMVFVRGPEQCDIQHFVEKVVFWLHDSFPKPRRVCKEPPYKVEESGYAGFIMPIEVHFKNKEEPRKVCFTYDLFLNLEGNPPVNHLRCEKLTFNNPTTEFRYKLLRAGGVMVMPEGADTVSRPSPDYPMLPTIPLSAFSDPKKTKPSHGSKDANKESSKTSKPHKVTKEHRERPRKDSESKSSSKELEREQAKSSKDTSRKLGEGRLPKEEKAPPPKAAFKEPKMALKETKLESTSPKGGPPPPPPPPPRASSKRPATADSPKPSAKKQKKSSSKGSRSAPGTSPRTSSSSSFSDKKPAKDKSSTRGEKVKAESEPREAKKALEVEESNSEDEASFKSESAQSSPSNSSSSSDSSSDSDFEPSQNHSQGPLRSMVEDLQSEESDEDDSSSGEEAAGKTNPGRDSRLSFSDSESDNSADSSLPSREPPPPQKPPPPNSKVSGRRSPESCSKPEKILKKGTYDKAYTDELVELHRRLMALRERNVLQQIVNLIEETGHFNVTNTTFDFDLFSLDETTVRKLQSCLEAVAT

SEQ ID NO: 37 - protein, 673 aa, Homo sapiens
  REGION 1..673: note = forkhead box protein O3 (FOXO3) [Homo sapiens] (aa)
MAEAPASPAPLSPLEVELDPEFEPQSRPRSCTWPLQRPELQASPAKPSGETAADSMIPEEEDDEDDEDGGGRAGSAMAIGGGGGSGTLGSGLLLEDSARVLAPGGQDPGSGPATAAGGLSGGTQALLQPQQPLPPPQPGAAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQTAPESADDSPSQLSKWPGSPTSRSSDELDAWTDFRSRTNSNASTVSGRLSPIMASTELDEVQDDDAPLSPMLYSSSASLSPSVSKPCTVELPRLTDMAGTMNLNDGLTENLMDDLLDNITLPPSQPSPTGGLMQRSSSFPYTTKGSGLGSPTSSFNSTVFGPSSLNSLRQSPMQTIQENKPATFSSMSHYGNQTLQDLLTSDSLSHSDVMMTQSDPLMSQASTAVSAQNSRRNVMLRNDPMMSFAAQPNQGSLVNQNLLHHQHQTQGALGGSRALSNSVSNMGLSESSSLGSAKHQQQSPVSQSMQTLSDSLSGSSLYSTSANLPVMGHEKFPSDLDLDMFNGSLECDMESIIRSELMDADGLDFNFDSLISTQNVVGLNVGNFTGAKQASSQSWVPG

SEQ ID NO: 38 - protein, 352 aa, Homo sapiens
  REGION 1..352: note = hematopoietic SH2 domain-containing protein (HSH2D) isoform 1 [Homo sapiens] (aa)
MTEAGKLPLPLPPRLDWFVHTQMGQLAQDGVPEWFHGAISREDAENLLESQPLGSFLIRVSHSHVGYTLSYKAQSSCCHFMVKLLDDGTFMIPGEKVAHTSLDALVTFHQQKPIEPRRELLTQPCRQKDPANVDYEDLFLYSNAVAEEAACPVSAPEEASPKPVLCHQSKERKPSAEMNRITTKEATSSCPPKSPLGETRQKLWRSLKMLPERGQRVRQQLKSHLATVNLSSLLDVRRSTVISGPGTGKGSQDHSGDPTSGDRGYTDPCVATSLKSPSQPQAPKDRKVPTRKAERSVSCIEVTPGDRSWHQMVVRALSSQESKPEHQGLAEPENDQLPEEYQQPPPFAPGYC

SEQ ID NO: 39 - protein, 1533 aa, Homo sapiens
  REGION 1..1533: note = nuclear receptor coactivator 2 (NCOA2) isoform X1 [Homo sapiens] (aa)
MSGMGENTSDPSRAETRKRKECPDQLGPSPKRNTEKRNREQENKYIEELAELIFANFNDIDNFNFKPDKCAILKETVKQIRQIKEQEKAAAANIDEVQKSDVSSTGQGVIDKDALGPMMLEALDGFFFVVNLEGNVVFVSENVTQYLRYNQEELMNKSVYSILHVGDHTEFVKNLLPKSIVNGGSWSGEPPRRNSHTFNCRMLVKPLPDSEEEGHDNQEAHQKYETMQCFAVSQPKSIKEEGEDLQSCLICVARRVPMKERPVLPSSESFTTRQDLQGKITSLDTSTMRAAMKPGWEDLVRRCIQKFHAQHEGESVSYAKRHHHEVLRQGLAFSQIYRFSLSDGTLVAAQTKSKLIRSQTTNEPQLVISLHMLHREQNVCVMNPDLTGQTMGKPLNPISSNSPAHQALCSGNPGQDMTLSSNINFPINGPKEQMGMPMGRFGGSGGMNHVSGMQATTPQGSNYALKMNSPSQSSPGMNPGQPTSMLSPRHRMSPGVAGSPRIPPSQFSPAGSLHSPVGVCSSTGNSHSYTNSSLNALQALSEGHGVSLGSSLASPDLKMGNLQNSPVNMNPPPLSKMGSLDSKDCFGLYGEPSEGTTGQAESSCHPGEQKETNDPNLPPAVSSERADGQSRLHDSKGQTKLLQLLTTKSDQMEPSPLASSLSDTNKDSTGSLPGSGSTHGTSLKEKHKILHRLLQDSSSPVDLAKLTAEATGKDLSQESSSTAPGSEVTIKQEPVSPKKKENALLRYLLDKDDTKDIGLPEITPKLERLDSKTDPASNTKLIAMKTEKEEMSFEPGDQPGSELDNLEEILDDLQNSQLPQLFPDTRPGAPAGSVDKQAIINDLMQLTAENSPVTPVGAQKTALRISQSTFNNPRPGQLGRLLPNQNLPLDITLQSPTGAGPFPPIRNSSPYSVIPQPGMMGNQGMIGNQGNLGNSSTGMIGNSASRPTMPSGEWAPQSSAVRVTCAATTSAMNRPVQGGMIRNPAASIPMRPSSQPGQRQTLQSQVMNIGPSELEMNMGGPQYSQQQAPPNQTAPWPESILPIDQASFASQNRQPFGSSPDDLLCPHPAAESPSDEGALLDQLYLALRNFDGLEEIDRALGIPELVSQSQAVDPEQFSSQDSNIMLEQKAPVFPQQYASQAQMAQGSYSPMQDPNFHTMGQRPSYATLRMQPRPGLRPTGLVQNQPNQLRLQLQHRLQAQQNRQPLMNQISNVSNVNLTLRPGVPTQAPINAQMLAQRQREILNQHLRQRQMHQQQQVQQRTLMMRGQGLNMTPSMVAPSGMPATMSNPRIPQANAQQFPFPPNYGTGLRSPPPFTSPFSPVSPSVGSQLLSHSSLHGSQMNLANQGMIGNLGGQLGPVRSPQVQHSTFQALSSGISQQPDPGFTGATTPQSPLMSPRMAHTQSPMMQQSQANPAYQAPSDINGWAQGNMGGNSMFSQQSPPHFGQQANTSMYSNNMNINVSMATNTGGMSSMNQMTGQISMTSVTSVPTSGLSSMGPEQVNDPALRGGNLFPNQLPGMDMIKQEGDTTRKYC

SEQ ID NO: 40 - protein, 1424 aa, Homo sapiens
  REGION 1..1424: note = nuclear receptor coactivator 3 (NCOA3) isoform a [Homo sapiens] (aa)
MSGLGENLDPLASDSRKRKLPCDTPGQGLTCSGEKRRREQESKYIEELAELISANLSDIDNFNVKPDKCAILKETVRQIRQIKEQGKTISNDDDVQKADVSSTGQGVIDKDSLGPLLLQALDGFLFVVNRDGNIVFVSENVTQYLQYKQEDLVNTSVYNILHEEDRKDFLKNLPKSTVNGVSWTNETQRQKSHTFNCRMLMKTPHDILEDINASPEMRQRYETMQCFALSQPRAMMEEGEDLQSCMICVARRITTGERTFPSNPESFITRHDLSGKVVNIDTNSLRSSMRPGFEDIIRRCIQRFFSLNDGQSWSQKRHYQEAYLNGHAETPVYRFSLADGTIVTAQTKSKLFRNPVTNDRHGFVSTHFLQREQNGYRPNPNPVGQGIRPPMAGCNSSVGGMSMSPNQGLQMPSSRAYGLADPSTTGQMSGARYGGSSNIASLTPGPGMQSPSSYQNNNYGLNMSSPPHGSPGLAPNQQNIMISPRNRGSPKIASHQFSPVAGVHSPMASSGNTGNHSFSSSSLSALQAISEGVGTSLLSTLSSPGPKLDNSPNMNITQPSKVSNQDSKSPLGFYCDQNPVESSMCQSNSRDHLSDKESKESSVEGAENQRGPLESKGHKKLLQLLTCSSDDRGHSSLTNSPLDSSCKESSVSVTSPSGVSSSTSGGVSSTSNMHGSLLQEKHRILHKLLQNGNSPAEVAKITAEATGKDTSSITSCGDGNVVKQEQLSPKKKENNALLRYLLDRDDPSDALSKELQPQVEGVDNKMSQCTSSTIPSSSQEKDPKIKTETSEEGSGDLDNLDAILGDLTSSDFYNNSISSNGSHLGTKQQVFQGTNSLGLKSSQSVQSIRPPYNRAVSLDSPVSVGSSPPVKNISAFPMLPKQPMLGGNPRMMDSQENYGSSMGGPNRNVTVTQTPSSGDWGLPNSKAGRMEPMNSNSMGRPGGDYNTSLPRPALGGSIPTLPLRSNSIPGARPVLQQQQQMLQMRPGEIPMGMGANPYGQAAASNQLGSWPDGMLSMEQVSHGTQNRPLLRNSLDDLVGPPSNLEGQSDERALLDQLHTLLSNTDATGLEEIDRALGIPELVNQGQALEPKQDAFQGQEAAVMMDQKAGLYGQTYPAQGPPMQGGFHLQGQSPSFNSMMNQMNQQGNFPLQGMHPRANIMRPRTNTPKQLRMQLQQRLQGQQFLNQSRQALELKMENPTAGGAAVMRPMMQPQVSSQQGFLNAQMVAQRSRELLSHHFRQQRVAMMMQQQQQQQQQQQQQQQQQQQQQQQQQQQQQTQAFSPPPNVTASPSMDGLLAGPTMPQAPPQQFPYQPNYGMGQQPDPAFGRVSSPPNAMMSSRMGPSQNPMMQHPQAASIYQSSEMKGWPSGNLARNSSFSQQQFAHQGNPAVYSMVHMNGSSGHMGQMNMNPMPMSGMPMGPDQKYC

SEQ ID NO: 41 - protein, 269 aa, Homo sapiens
  REGION 1..269: note = proteasome subunit alpha type-1 (PSA1) isoform 1 [Homo sapiens] (aa)
MQLSKVKFRNQYDNDVTVWSPQGRIHQIEYAMEAVKQGSATVGLKSKTHAVLVALKRAQSELAAHQKKILHVDNHIGISIAGLTADARLLCNFMRQECLDSRFVFDRPLPVSRLVSLIGSKTQIPTQRYGRRPYGVGLLIAGYDDMGPHIFQTCPSANYFDCRAMSIGARSQSARTYLERHMSEFMECNLNELVKHGLRALRETLPAEQDLTTKNVSIGIVGKDLEFTIYDDDDVSPFLEGLEERPQRKAQPAQPADEPAEKADEPMEH

SEQ ID NO: 42 - protein, 419 aa, Homo sapiens
  REGION 1..419: note = pygopus homolog 1 (PYGO1) isoform 1 [Homo sapiens] (aa)
MPAENSPAPAYKVSSHGGDSGLDGLGGPGVQLGSPDKKKRKANTQGPSFPPLSEYAPPPNPNSDHLVAANPFDDNYNTISYKPLPSSNPYLGPGYPGFGGYSTFRMPPHVPPRMSSPYCGPYSLRNQPHPFPQNPLGMGFNRPHAFNFGPHDNSSFGNPSYNNALSQNVNMPNQHFRQNPAENFSQIPPQNASQVSNPDLASNFVPGNNSNFTSPLESNHSFIPPPNTFGQAKAPPPKQDFTQGATKNTNQNSSAHPPHLNMDDTVNQSNIELKNVNRNNAVNQENSRSSSTEATNNNPANGTQNKPRQPRGAADACTTEKSNKSSLHPNRHGHSSSDPVYPCGICTNEVNDDQDAILCEASCQKWFHRICTGMTETAYGLLTAEASAVWGCDTCMADKDVQLMRTRETFGPSAVGSDA

SEQ ID NO: 43 - protein, 530 aa, Homo sapiens
  REGION 1..530: note = RNA-binding protein 39 (RBM39) isoform a [Homo sapiens] (aa)
MADDIDIEAMLEAPYKKDENKLSSANGHEERSKKRKKSKSRSRSHERKRSKSKERKRSRDRERKKSKSRERKRSRSKERRRSRSRSRDRRFRGRYRSPYSGPKFNSAIRGKIGLPHSIKLSRRRSRSKSPFRKDKSPVREPIDNLTPEERDARTVFCMQLAARIRPRDLEEFFSTVGKVRDVRMISDRNSRRSKGIAYVEFVDVSSVPLAIGLTGQRVLGVPIIVQASQAEKNRAAAMANNLQKGSAGPMRLYVGSLHFNITEDMLRGIFEPFGRIESIQLMMDSETGRSKGYGFITFSDSECAKKALEQLNGFELAGRPMKVGHVTERTDASSASSFLDSDELERTGIDLGTTGRLQLMARLAEGTGLQIPPAAQQALQMSGSLAFGAVAEFSFVIDLQTRLSQQTEASALAAAASVQPLATQCFQLSNMFNPQTEEEVGWDTEIKDDVIEECNKHGGVIHIYVDKNSAQGNVYVKCPSIAAAIAAVNALHGRWFAGKMITAAYVPLPTYHNLFPDSMTATQLLVPSRR

SEQ ID NO: 44 - protein, 4834 aa, Homo sapiens
  REGION 1..4834: note = E3 ubiquitin-protein ligase HERC2 (HERC2) [Homo sapiens] (aa)
MPSESFCLAAQARLDSKWLKTDIQLAFTRDGLCGLWNEMVKDGEIVYTGTESTQNGELPPRKDDSVEPSGTKKEDLNDKEKKDEEETPAPIYRAKSILDSWVWGKQPDVNELKECLSVLVKEQQALAVQSATTTLSALRLKQRLVILERYFIALNRTVFQENVKVKWKSSGISLPPVDKKSSRPAGKGVEGLARVGSRAALSFAFAFLRRAWRSGEDADLCSELLQESLDALRALPEASLFDESTVSSVWLEVVERATRFLRSVVTGDVHGTPATKGPGSIPLQDQHLALAILLELAVQRGTLSQMLSAILLLLQLWDSGAQETDNERSAQGTSAPLLPLLQRFQSIICRKDAPHSEGDMHLLSGPLSPNESFLRYLTLPQDNELAIDLRQTAVVVMAHLDRLATPCMPPLCSSPTSHKGSLQEVIGWGLIGWKYYANVIGPIQCEGLANLGVTQIACAEKRFLILSRNGRVYTQAYNSDTLAPQLVQGLASRNIVKIAAHSDGHHYLALAATGEVYSWGCGDGGRLGHGDTVPLEEPKVISAFSGKQAGKHVVHIACGSTYSAAITAEGELYTWGRGNYGRLGHGSSEDEAIPMLVAGLKGLKVIDVACGSGDAQTLAVTENGQVWSWGDGDYGKLGRGGSDGCKTPKLIEKLQDLDVVKVRCGSQFSIALTKDGQVYSWGKGDNQRLGHGTEEHVRYPKLLEGLQGKKVIDVAAGSTHCLALTEDSEVHSWGSNDQCQHFDTLRVTKPEPAALPGLDTKHIVGIACGPAQSFAWSSCSEWSIGLRVPFVVDICSMTFEQLDLLLRQVSEGMDGSADWPPPQEKECVAVATLNLLRLQLHAAISHQVDPEFLGLGLGSILLNSLKQTVVTLASSAGVLSTVQSAAQAVLQSGWSVLLPTAEERARALSALLPCAVSGNEVNISPGRRFMIDLLVGSLMADGGLESALHAAITAEIQDIEAKKEAQKEKEIDEQEANASTFHRSRTPLDKDLINTGICESSGKQCLPLVQLIQQLLRNIASQTVARLKDVARRISSCLDFEQHSRERSASLDLLLRFQRLLISKLYPGESIGQTSDISSPELMGVGSLLKKYTALLCTHIGDILPVAASIASTSWRHFAEVAYIVEGDFTGVLLPELVVSIVLLLSKNAGLMQEAGAVPLLGGLLEHLDRFNHLAPGKERDDHEELAWPGIMESFFTGQNCRNNEEVTLIRKADLENHNKDGGFWTVIDGKVYDIKDFQTQSLTGNSILAQFAGEDPVVALEAALQFEDTRESMHAFCVGQYLEPDQEIVTIPDLGSLSSPLIDTERNLGLLLGLHASYLAMSTPLSPVEIECAKWLQSSIFSGGLQTSQIHYSYNEEKDEDHCSSPGGTPASKSRLCSHRRALGDHSQAFLQAIADNNIQDHNVKDFLCQIERYCRQCHLTTPIMFPPEHPVEEVGRLLLCCLLKHEDLGHVALSLVHAGALGIEQVKHRTLPKSVVDVCRVVYQAKCSLIKTHQEQGRSYKEVCAPVIERLRFLFNELRPAVCNDLSIMSKFKLLSSLPRWRRIAQKIIRERRKKRVPKKPESTDDEEKIGNEESDLEEACILPHSPINVDKRPIAIKSPKDKWQPLLSTVTGVHKYKWLKQNVQGLYPQSPLLSTIAEFALKEEPVDVEKMRKCLLKQLERAEVRLEGIDTILKLASKNFLLPSVQYAMFCGWQRLIPEGIDIGEPLTDCLKDVDLIPPFNRMLLEVTFGKLYAWAVQNIRNVLMDASAKFKELGIQPVPLQTITNENPSGPSLGTIPQARFLLVMLSMLTLQHGANNLDLLLNSGMLALTQTALRLIGPSCDNVEEDMNASAQGASATVLEETRKETAPVQLPVSGPELAAMMKIGTRVMRGVDWKWGDQDGPPPGLGRVIGELGEDGWIRVQWDTGSTNSYRMGKEGKYDLKLAELPAAAQPSAEDSDTEDDSEAEQTERNIHPTAMMFTSTINLLQTLCLSAGVHAEIMQSEATKTLCGLLRMLVESGTTDKTSSPNRLVYREQHRSWCTLGFVRSIALTPQVCGALSSPQWITLLMKVVEGHAPFTATSLQRQILAVHLLQAVLPSWDKTERARDMKCLVEKLFDFLGSLLTTCSSDVPLLRESTLRRRRVRPQASLTATHSSTLAEEVVALLRTLHSLTQWNGLINKYINSQLRSITHSFVGRPSEGAQLEDYFPDSENPEVGGLMAVLAVIGGIDGRLRLGGQVMHDEFGEGTVTRITPKGKITVQFSDMRTCRVCPLNQLKPLPAVAFNVNNLPFTEPMLSVWAQLVNLAGSKLEKHKIKKSTKQAFAGQVDLDLLRCQQLKLYILKAGRALLSHQDKLRQILSQPAVQETGTVHTDDGAVVSPDLGDMSPEGPQPPMILLQQLLASATQPSPVKAIFDKQELEAAALAVCQCLAVESTHPSSPGFEDCSSSEATTPVAVQHIRPARVKRRKQSPVPALPIVVQLMEMGFSRRNIEFALKSLTGASGNASSLPGVEALVGWLLDHSDIQVTELSDADTVSDEYSDEEVVEDVDDAAYSMSTGAVVTESQTYKKRADFLSNDDYAVYVRENIQVGMMVRCCRAYEEVCEGDVGKVIKLDRDGLHDLNVQCDWQQKGGTYWVRYIHVELIGYPPPSSSSHIKIGDKVRVKASVTTPKYKWGSVTHQSVGVVKAFSANGKDIIVDFPQQSHWTGLLSEMELVPSIHPGVTCDGCQMFPINGSRFKCRNCDDFDFCETCFKTKKHNTRHTFGRINEPGQSAVFCGRSGKQLKRCHSSQPGMLLDSWSRMVKSLNVSSSVNQASRLIDGSEPCWQSSGSQGKHWIRLEIFPDVLVHRLKMIVDPADSSYMPSLVVVSGGNSLNNLIELKTININPSDTTVPLLNDCTEYHRYIEIAIKQCRSSGIDCKIHGLILLGRIRAEEEDLAAVPFLASDNEEEEDEKGNSGSLIRKKAAGLESAATIRTKVFVWGLNDKDQLGGLKGSKIKVPSFSETLSALNVVQVAGGSKSLFAVTVEGKVYACGEATNGRLGLGISSGTVPIPRQITALSSYVVKKVAVHSGGRHATALTVDGKVFSWGEGDDGKLGHFSRMNCDKPRLIEALKTKRIRDIACGSSHSAALTSSGELYTWGLGEYGRLGHGDNTTQLKPKMVKVLLGHRVIQVACGSRDAQTLALTDEGLVFSWGDGDFGKLGRGGSEGCNIPQNIERLNGQGVCQIECGAQFSLALTKSGVVWTWGKGDYFRLGHGSDVHVRKPQVVEGLRGKKIVHVAVGALHCLAVTDSGQVYAWGDNDHGQQGNGTTTVNRKPTLVQGLEGQKITRVACGSSHSVAWTTVDVATPSVHEPVLFQTARDPLGASYLGVPSDADSSAASNKISGASNSKPNRPSLAKILLSLDGNLAKQQALSHILTALQIMYARDAVVGALMPAAMIAPVECPSFSSAAPSDASAMASPMNGEECMLAVDIEDRLSPNPWQEKREIVSSEDAVTPSAVTPSAPSASARPFIPVTDDLGAASIIAETMTKTKEDVESQNKAAGPEPQALDEFTSLLIADDTRVVVDLLKLSVCSRAGDRGRDVLSAVLSGMGTAYPQVADMLLELCVTELEDVATDSQSGRLSSQPVVVESSHPYTDDTSTSGTVKIPGAEGLRVEFDRQCSTERRHDPLTVMDGVNRIVSVRSGREWSDWSSELRIPGDELKWKFISDGSVNGWGWRFTVYPIMPAAGPKELLSDRCVLSCPSMDLVTCLLDFRLNLASNRSIVPRLAASLAACAQLSALAASHRMWALQRLRKLLTTEFGQSININRLLGENDGETRALSFTGSALAALVKGLPEALQRQFEYEDPIVRGGKQLLHSPFFKVLVALACDLELDTLPCCAETHKWAWFRRYCMASRVAVALDKRTPLPRLFLDEVAKKIRELMADSENMDVLHESHDIFKREQDEQLVQWMNRRPDDWTLSAGGSGTIYGWGHNHRGQLGGIEGAKVKVPTPCEALATLRPVQLIGGEQTLFAVTADGKLYATGYGAGGRLGIGGTESVSTPTLLESIQHVFIKKVAVNSGGKHCLALSSEGEVYSWGEAEDGKLGHGNRSPCDRPRVIESLRGIEVVDVAAGGAHSACVTAAGDLYTWGKGRYGRLGHSDSEDQLKPKLVEALQGHRVVDIACGSGDAQTLCLTDDDTVWSWGDGDYGKLGRGGSDGCKVPMKIDSLTGLGVVKVECGSQFSVALTKSGAVYTWGKGDYHRLGHGSDDHVRRPRQVQGLQGKKVIAIATGSLHCVCCTEDGEVYTWGDNDEGQLGDGTTNAIQRPRLVAALQGKKVNRVACGSAHTLAWSTSKPASAGKLPAQVPMEYNHLQEIPIIALRNRLLLLHHLSELFCPCIPMFDLEGSLDETGLGPSVGFDTLRGILISQGKEAAFRKVVQATMVRDRQHGPVVELNRIQVKRSRSKGGLAGPDGTKSVFGQMCAKMSSFGPDSLLLPHRVWKVKFVGESVDDCGGGYSESIAEICEELQNGLTPLLIVTPNGRDESGANRDCYLLSPAARAPVHSSMFRFLGVLLGIAIRTGSPLSLNLAEPVWKQLAGMSLTIADLSEVDKDFIPGLMYIRDNEATSEEFEAMSLPFTVPSASGQDIQLSSKHTHITLDNRAEYVRLAINYRLHEFDEQVAAVREGMARVVPVPLLSLFTGYELETMVCGSPDIPLHLLKSVATYKGIEPSASLIQWFWEVMESFSNTERSLFLRFVWGRTRLPRTIADFRGRDFVIQVLDKYNPPDHFLPESYTCFFLLKLPRYSCKQVLEEKLKYAIHFCKSIDTDDYARIALTGEPAADDSSDDSDNEDVDSFASDSTQDYLTGH

SEQ ID NO: 45 - protein, 3703 aa, Homo sapiens
  REGION 1..3703: note = dystrophin (DMD) isoform X1 [Homo sapiens] (aa)
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGHNVGSLFHMADDLGRAMESLVSVMTDEEGAE

SEQ ID NO: 46 - protein, 2471 aa, Homo sapiens
  REGION 1..2471: note = NOTCH2 (NOTC2) [Homo sapiens] (aa)
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCNQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 47 - protein, 2414 aa, synthetic construct
  REGION 1..2414: note = p300 (aa)
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 48 - DNA, 57 bp, synthetic construct
  misc_feature 1..57: note = P2A sequence (nt)
  source 1..57: mol_type = other DNA
gcaacaaacttctctctgctgaaacaagccggagatgtcgaagagaatcctggaccg

SEQ ID NO: 49 - protein, 19 aa, synthetic construct
  REGION 1..19: note = P2A sequence (aa)
ATNFSLLKQAGDVEENPGP

SEQ ID NO: 50-60 - skipped (11 entries)

SEQ ID NO: 61 - protein, 22 aa, synthetic construct
  REGION 1..22: note = SunTag GCN4 peptide (aa)
LLPKNYHLENEVARLKKLVGER

SEQ ID NO: 62 - protein, 5 aa, synthetic construct
  REGION 1..5: note = GGSGG linker (aa)
GGSGG

SEQ ID NO: 63 - protein, 5 aa, synthetic construct
  REGION 1..5: note = GGGGG linker (aa)
GGGGG

SEQ ID NO: 64 - protein, 5 aa, synthetic construct
  REGION 1..5: note = GGAGG linker (aa)
GGAGG

SEQ ID NO: 65 - protein, 7 aa, synthetic construct
  REGION 1..7: note = GGGGSSS linker (aa)
GGGGSSS

SEQ ID NO: 66 - protein, 7 aa, synthetic construct
  REGION 1..7: note = GGGGAAA linker (aa)
GGGGAAA

SEQ ID NO: 67 - protein, 50 aa, synthetic construct
  REGION 1..50: note = (GGGGS)n linker (aa), n is 1 to 10
GGGGSXXXXXXXXXXXXXXXXXXXXXXXXXXXXXXXXXXXXXXXXXXXXX

SEQ ID NO: 68 - DNA, 48 bp, synthetic construct
  misc_feature 1..48: note = nucleoplasmin NLS (nt)
  source 1..48: mol_type = other DNA
aaaaggccggcggccacgaaaaaggccggccaggcaaaaaagaaaaag

SEQ ID NO: 69 - protein, 16 aa, synthetic construct
  REGION 1..16: note = nucleoplasmin NLS (aa)
KRPAATKKAGQAKKKK

SEQ ID NO: 70 - protein, 7 aa, synthetic construct
  REGION 1..7: note = SV40 virus large T-antigen NLS
PKKKRKV

SEQ ID NO: 71 - protein, 9 aa, synthetic construct
  REGION 1..9: note = c-myc NLS (aa)
PAAKRVKLD

SEQ ID NO: 72 - protein, 11 aa, synthetic construct
  REGION 1..11: note = c-myc NLS (aa)
RQRRNELKRSP

SEQ ID NO: 73 - protein, 38 aa, synthetic construct
  REGION 1..38: note = hRNPA1 M9 NLS (aa)
NQSSNFGPMKGGNFGGRSSGPYGGGGQYFAKPRNQGGY

SEQ ID NO: 74 - protein, 42 aa, synthetic construct
  REGION 1..42: note = importin-alpha IBB domain NLS (aa)
RMRIZFKNKGKDTAELRRRRVEVSVELRKAKKDEQILKRRNV

SEQ ID NO: 75 - protein, 8 aa, synthetic construct
  REGION 1..8: note = myoma T protein NLS (aa)
VSRKRPRP

SEQ ID NO: 76 - protein, 8 aa, synthetic construct
  REGION 1..8: note = myoma T protein NLS (aa)
PPKKARED

SEQ ID NO: 77 - protein, 8 aa, Homo sapiens
  REGION 1..8: note = human p53 NLS (aa)
PQPKKKPL

SEQ ID NO: 78 - protein, 12 aa, Mus musculus
  REGION 1..12: note = mouse c-abl IV NLS (aa)
SALIKKKKKMAP

SEQ ID NO: 79 - protein, 5 aa, synthetic construct
  REGION 1..5: note = influenza virus NS1 NLS (aa)
DRLRR

SEQ ID NO: 80 - protein, 7 aa, synthetic construct
  REGION 1..7: note = influenza virus NS1 NLS (aa)
PKQKKRK

SEQ ID NO: 81 - protein, 10 aa, Hepatitis B virus
  REGION 1..10: note = Hepatitis virus delta antigen NLS (aa)
RKLKKKIKKL

SEQ ID NO: 82 - protein, 10 aa, Mus musculus
  REGION 1..10: note = mouse Mx1 protein MNLS (aa)
REKKKFLKRR

SEQ ID NO: 83 - protein, 20 aa, Homo sapiens
  REGION 1..20: note = human poly(ADP-ribose) polymerase NLS (aa)
KRKGDEVDGVDEVAKKKSKK

SEQ ID NO: 84 - protein, 17 aa, Homo sapiens
  REGION 1..17: note = steroid hormone receptors (human) glucocorticoid NLS (aa)
RKCLQAGMNLEARKTKK

SEQ ID NO: 85 - DNA, 1716 bp, synthetic construct
  misc_feature 1..1716: note = N term dSpCas9 N terminal fragement (nt)
  source 1..1716: mol_type = other DNA
gacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaa

SEQ ID NO: 86 - protein, 572 aa, synthetic construct
  REGION 1..572: note = N term dSpCas9 N terminal fragement
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIE

SEQ ID NO: 87 - DNA, 255 bp, synthetic construct
  misc_feature 1..255: note = N term Npu Intein (nt)
  source 1..255: mol_type = other DNA
tgcctgtcctacgagacagagatcctgacagtggagtatggcctgctgccaatcggcaagatcgtggagaagaggatcgagtgtaccgtgtactctgtggataacaatggcaacatctatacacagcccgtggcacagtggcacgataggggagagcaggaggtgttcgagtattgcctggaggacggcagcctgatcagggcaaccaaggaccacaagttcatgacagtggatggccagatgctgcccatcgac

SEQ ID NO: 88 - protein, 85 aa, synthetic construct
  REGION 1..85: note = N term Npu Intein (aa)
CLSYETEILTVEYGLLPIGKIVEKRIECTVYSVDNNGNIYTQPVAQWHDRGEQEVFEYCLEDGSLIRATKDHKFMTVDGQMLPID

SEQ ID NO: 89 - DNA, 2598 bp, synthetic construct
  misc_feature 1..2598: note = dSpCas9-573-C Intein (nt)
  source 1..2598: mol_type = other DNA
atgaaacggacagccgacggaagcgagttcgagtcaccaaagaagaagcggaaagtcatcaagattgctacacggaaatacctgggaaagcagaacgtgtacgacatcggcgtggagcgggatcacaacttcgccctgaagaatggctttatcgccagcaattgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacaaaaggccggcggccacgaaaaaggccggccaggcaaaaaagaaaaagtga

SEQ ID NO: 90 - protein, 865 aa, synthetic construct
  REGION 1..865: note = dSpCas9-573-C Intein (aa)
MKRTADGSEFESPKKKRKVIKIATRKYLGKQNVYDIGVERDHNFALKNGFIASNCFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 91 - DNA, 105 bp, synthetic construct
  misc_feature 1..105: note = C term Npu Intein (nt)
  source 1..105: mol_type = other DNA
atcaagattgctacacggaaatacctgggaaagcagaacgtgtacgacatcggcgtggagcgggatcacaacttcgccctgaagaatggctttatcgccagcaat

SEQ ID NO: 92 - protein, 35 aa, synthetic construct
  REGION 1..35: note = C term Npu Intein (aa)
IKIATRKYLGKQNVYDIGVERDHNFALKNGFIASN

SEQ ID NO: 93 - DNA, 2385 bp, synthetic construct
  misc_feature 1..2385: note = C term dSpCas9 C terminal fragment (nt)
  source 1..2385: mol_type = other DNA
tgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 94 - protein, 795 aa, synthetic construct
  REGION 1..795: note = C term dSpCas9 C terminal fragment (aa)
CFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 95 - DNA, 240 bp, synthetic construct
  misc_feature 1..240: note = extended linker (nt)
  source 1..240: mol_type = other DNA
ggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgag

SEQ ID NO: 96 - protein, 80 aa, synthetic construct
  REGION 1..80: note = extended linker (aa)
GGPSSGAPPPSGGSPAGSPTSTEEGTSESATPESGPGTSTEPSEGSAPGSPAGSPTSTEEGTSTEPSEGSAPGTSTEPSE

SEQ ID NO: 97 - DNA, 171 bp, synthetic construct
  misc_feature 1..171: note = PYGO1 transcriptional activation domain (nt)
  source 1..171: mol_type = other DNA
ccctgcggcatctgcaccaacgaggtgaacgacgaccaggacgccatcctgtgcgaggccagctgccagaagtggttccacagaatctgcaccggcatgaccgagaccgcctacggcctgctgaccgccgaggccagcgctgtgtggggatgcgacacctgcatggccgac

SEQ ID NO: 98 - DNA, 186 bp, synthetic construct
  misc_feature 1..186: note = ENL transcriptional activation domain (nt)
  source 1..186: mol_type = other DNA
gcctacaccgacgagctggtggagctgcacagaagactgatggccctgagagagagaaacgtgctgcagcagatcgtgaacctgatcgaggagaccggccacttcaacgtgaccaacaccaccttcgacttcgacctgttcagcctggacgagaccaccgtgagaaagctgcagagctgcctggag

SEQ ID NO: 99 - DNA, 126 bp, synthetic construct
  misc_feature 1..126: note = FOXO3 transcriptional activation domain, codon optimized (nt)
  source 1..126: mol_type = other DNA
cacgagaaattcccctccgatttggatctggatatgttcaacgggtcacttgaatgtgacatggagagcattatacgctccgaacttatggacgcagacggtcttgactttaactttgattccctc

SEQ ID NO: 100 - DNA, 126 bp, synthetic construct
  misc_feature 1..126: note = FOXO3 transcriptional activation domain (nt)
  source 1..126: mol_type = other DNA
cacgagaagttccccagcgacctggacctggacatgttcaacggcagcctggagtgcgacatggagagcatcatcagaagcgagctgatggacgccgacggcctggacttcaacttcgacagcctg

SEQ ID NO: 101 - DNA, 147 bp, synthetic construct
  misc_feature 1..147: note = NCOA3 transcriptional activation domain (nt)
  source 1..147: mol_type = other DNA
gagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 102 - DNA, 147 bp, synthetic construct
  misc_feature 1..147: note = NCOA3 transcriptional activation domain, codon optimized (nt)
  source 1..147: mol_type = other DNA
gaagggcaaagcgacgagcgagcactcttggatcagctccacacgctgctctcaaacactgatgccactggcttggaagagatcgatcgagcattgggaatcccagagcttgtaaatcaaggtcaggcgctggaacctaaacaggat

SEQ ID NO: 103 - DNA, 228 bp, synthetic construct
  misc_feature 1..228: note = NCOA3 transcriptional activation domain, codon optimized (nt)
  source 1..228: mol_type = other DNA
tggttccacggcgccatcagcagagaggacgccgagaacctgctggagagccagcccctgggcagcttcctgatcagagtgagccacagccacgtgggctacaccctgagctacaaggcccagagcagctgctgccacttcatggtgaagctgctggacgacggcaccttcatgatccccggcgagaaggtggcccacaccagcctggacgccctggtgaccttccac

SEQ ID NO: 104 - DNA, 141 bp, synthetic construct
  misc_feature 1..141: note = NCOA2 transcriptional activation domain (nt)
  source 1..141: mol_type = other DNA
gaaagccccagcgacgagggcgctctgctggaccagctgtacctggccctgagaaacttcgacggcctggaggagatcgacagagccctgggcatccccgagctggtgagccagagccaggccgtggaccccgagcagttc

SEQ ID NO: 105 - DNA, 111 bp, synthetic construct
  misc_feature 1..111: note = NOTCH2 transcriptional activation domain (nt)
  source 1..111: mol_type = other DNA
gacatcgacgagtgcgccagcaacccctgcagaaagggcgccacctgcatcaacggcgtgaacggcttcagatgcatctgccccgagggcccccaccaccccagctgctac

SEQ ID NO: 106 - DNA, 18 bp, synthetic construct
  misc_feature 1..18: note = Multipartite linker 1 (nt)
  source 1..18: mol_type = other DNA
aacggaggtggaggctca

SEQ ID NO: 107 - DNA, 15 bp, synthetic construct
  misc_feature 1..15: note = Multipartite linker 2 (nt)
  source 1..15: mol_type = other DNA
ggtggaggaggcagt

SEQ ID NO: 108 - DNA, 45 bp, synthetic construct
  misc_feature 1..45: note = Multipartite linker 3 (nt)
  source 1..45: mol_type = other DNA
ggaggaggaggttctgggggtggaggtagcggtggtggaggtagt

SEQ ID NO: 109 - DNA, 336 bp, synthetic construct
  misc_feature 1..336: note = Bipartite activator - PYGO1 NCOA3 (nt)
  source 1..336: mol_type = other DNA
ccctgcggcatctgcaccaacgaggtgaacgacgaccaggacgccatcctgtgcgaggccagctgccagaagtggttccacagaatctgcaccggcatgaccgagaccgcctacggcctgctgaccgccgaggccagcgctgtgtggggatgcgacacctgcatggccgacaacggaggtggaggctcagagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 110 - DNA, 276 bp, synthetic construct
  misc_feature 1..276: note = Bipartite activator - NOTCH2 NCOA3 (nt)
  source 1..276: mol_type = other DNA
gacatcgacgagtgcgccagcaacccctgcagaaagggcgccacctgcatcaacggcgtgaacggcttcagatgcatctgccccgagggcccccaccaccccagctgctacaacggaggtggaggctcagagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 111 - DNA, 309 bp, synthetic construct
  misc_feature 1..309: note = Bipartite activator - NCOA3 NCOA3 (nt)
  source 1..309: mol_type = other DNA
gaagggcaaagcgacgagcgagcactcttggatcagctccacacgctgctctcaaacactgatgccactggcttggaagagatcgatcgagcattgggaatcccagagcttgtaaatcaaggtcaggcgctggaacctaaacaggatggtggaggaggcagtgagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 112 - DNA, 393 bp, synthetic construct
  misc_feature 1..393: note = Bipartite activator - HSH2D NCOA3 (nt)
  source 1..393: mol_type = other DNA
tggttccacggcgccatcagcagagaggacgccgagaacctgctggagagccagcccctgggcagcttcctgatcagagtgagccacagccacgtgggctacaccctgagctacaaggcccagagcagctgctgccacttcatggtgaagctgctggacgacggcaccttcatgatccccggcgagaaggtggcccacaccagcctggacgccctggtgaccttccacaacggaggtggaggctcagagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 113 - DNA, 288 bp, synthetic construct
  misc_feature 1..288: note = Bipartite activator - FOXO3 NCOA3 (nt)
  source 1..288: mol_type = other DNA
cacgagaagttccccagcgacctggacctggacatgttcaacggcagcctggagtgcgacatggagagcatcatcagaagcgagctgatggacgccgacggcctggacttcaacttcgacagcctgggtggaggaggcagtgagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 114 - DNA, 306 bp, synthetic construct
  misc_feature 1..306: note = Bipartite activator - NCOA2 NCOA3 (nt)
  source 1..306: mol_type = other DNA
gaaagccccagcgacgagggcgctctgctggaccagctgtacctggccctgagaaacttcgacggcctggaggagatcgacagagccctgggcatccccgagctggtgagccagagccaggccgtggaccccgagcagttcaacggaggtggaggctcagagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 115 - DNA, 351 bp, synthetic construct
  misc_feature 1..351: note = Bipartite activator - ENL NCOA3 (nt)
  source 1..351: mol_type = other DNA
gcctacaccgacgagctggtggagctgcacagaagactgatggccctgagagagagaaacgtgctgcagcagatcgtgaacctgatcgaggagaccggccacttcaacgtgaccaacaccaccttcgacttcgacctgttcagcctggacgagaccaccgtgagaaagctgcagagctgcctggagaacggaggtggaggctcagagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 116 - DNA, 315 bp, synthetic construct
  misc_feature 1..315: note = Bipartite activator - PYGO1 FOXO3 (nt)
  source 1..315: mol_type = other DNA
ccctgcggcatctgcaccaacgaggtgaacgacgaccaggacgccatcctgtgcgaggccagctgccagaagtggttccacagaatctgcaccggcatgaccgagaccgcctacggcctgctgaccgccgaggccagcgctgtgtggggatgcgacacctgcatggccgacaacggaggtggaggctcacacgagaagttccccagcgacctggacctggacatgttcaacggcagcctggagtgcgacatggagagcatcatcagaagcgagctgatggacgccgacggcctggacttcaacttcgacagcctg

SEQ ID NO: 117 - DNA, 255 bp, synthetic construct
  misc_feature 1..255: note = Bipartite activator - NOTCH2 FOXO3 (nt)
  source 1..255: mol_type = other DNA
gacatcgacgagtgcgccagcaacccctgcagaaagggcgccacctgcatcaacggcgtgaacggcttcagatgcatctgccccgagggcccccaccaccccagctgctacaacggaggtggaggctcacacgagaagttccccagcgacctggacctggacatgttcaacggcagcctggagtgcgacatggagagcatcatcagaagcgagctgatggacgccgacggcctggacttcaacttcgacagcctg

SEQ ID NO: 118 - DNA, 291 bp, synthetic construct
  misc_feature 1..291: note = Bipartite activator - NCOA3 FOXO3 (nt)
  source 1..291: mol_type = other DNA
gaagggcaaagcgacgagcgagcactcttggatcagctccacacgctgctctcaaacactgatgccactggcttggaagagatcgatcgagcattgggaatcccagagcttgtaaatcaaggtcaggcgctggaacctaaacaggataacggaggtggaggctcacacgagaagttccccagcgacctggacctggacatgttcaacggcagcctggagtgcgacatggagagcatcatcagaagcgagctgatggacgccgacggcctggacttcaacttcgacagcctg

SEQ ID NO: 119 - DNA, 372 bp, synthetic construct
  misc_feature 1..372: note = Bipartite activator - HSH2D FOXO3 (nt)
  source 1..372: mol_type = other DNA
tggttccacggcgccatcagcagagaggacgccgagaacctgctggagagccagcccctgggcagcttcctgatcagagtgagccacagccacgtgggctacaccctgagctacaaggcccagagcagctgctgccacttcatggtgaagctgctggacgacggcaccttcatgatccccggcgagaaggtggcccacaccagcctggacgccctggtgaccttccacaacggaggtggaggctcacacgagaagttccccagcgacctggacctggacatgttcaacggcagcctggagtgcgacatggagagcatcatcagaagcgagctgatggacgccgacggcctggacttcaacttcgacagcctg

SEQ ID NO: 120 - DNA, 270 bp, synthetic construct
  misc_feature 1..270: note = Bipartite activator - FOXO3 FOXO3 (nt)
  source 1..270: mol_type = other DNA
cacgagaaattcccctccgatttggatctggatatgttcaacgggtcacttgaatgtgacatggagagcattatacgctccgaacttatggacgcagacggtcttgactttaactttgattccctcaacggaggtggaggctcacacgagaagttccccagcgacctggacctggacatgttcaacggcagcctggagtgcgacatggagagcatcatcagaagcgagctgatggacgccgacggcctggacttcaacttcgacagcctg

SEQ ID NO: 121 - DNA, 285 bp, synthetic construct
  misc_feature 1..285: note = Bipartite activator - NCOA2 FOXO3 (nt)
  source 1..285: mol_type = other DNA
gaaagccccagcgacgagggcgctctgctggaccagctgtacctggccctgagaaacttcgacggcctggaggagatcgacagagccctgggcatccccgagctggtgagccagagccaggccgtggaccccgagcagttcaacggaggtggaggctcacacgagaagttccccagcgacctggacctggacatgttcaacggcagcctggagtgcgacatggagagcatcatcagaagcgagctgatggacgccgacggcctggacttcaacttcgacagcctg

SEQ ID NO: 122 - DNA, 330 bp, synthetic construct
  misc_feature 1..330: note = Bipartite activator - ENL FOXO3 (nt)
  source 1..330: mol_type = other DNA
gcctacaccgacgagctggtggagctgcacagaagactgatggccctgagagagagaaacgtgctgcagcagatcgtgaacctgatcgaggagaccggccacttcaacgtgaccaacaccaccttcgacttcgacctgttcagcctggacgagaccaccgtgagaaagctgcagagctgcctggagaacggaggtggaggctcacacgagaagttccccagcgacctggacctggacatgttcaacggcagcctggagtgcgacatggagagcatcatcagaagcgagctgatggacgccgacggcctggacttcaacttcgacagcctg

SEQ ID NO: 123 - DNA, 477 bp, synthetic construct
  misc_feature 1..477: note = Tripartite activator - PYGO1 FOXO3 NCOA3 (nt)
  source 1..477: mol_type = other DNA
ccctgcggcatctgcaccaacgaggtgaacgacgaccaggacgccatcctgtgcgaggccagctgccagaagtggttccacagaatctgcaccggcatgaccgagaccgcctacggcctgctgaccgccgaggccagcgctgtgtggggatgcgacacctgcatggccgacaacggaggtggaggctcacacgagaagttccccagcgacctggacctggacatgttcaacggcagcctggagtgcgacatggagagcatcatcagaagcgagctgatggacgccgacggcctggacttcaacttcgacagcctgggtggaggaggcagtgagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 124 - DNA, 417 bp, synthetic construct
  misc_feature 1..417: note = Tripartite activator - NOTCH2 FOXO3 NCOA3 (nt)
  source 1..417: mol_type = other DNA
gacatcgacgagtgcgccagcaacccctgcagaaagggcgccacctgcatcaacggcgtgaacggcttcagatgcatctgccccgagggcccccaccaccccagctgctacaacggaggtggaggctcacacgagaagttccccagcgacctggacctggacatgttcaacggcagcctggagtgcgacatggagagcatcatcagaagcgagctgatggacgccgacggcctggacttcaacttcgacagcctgggtggaggaggcagtgagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 125 - DNA, 453 bp, synthetic construct
  misc_feature 1..453: note = Tripartite activator - NCOA3 FOXO3 NCOA3 (nt)
  source 1..453: mol_type = other DNA
gaagggcaaagcgacgagcgagcactcttggatcagctccacacgctgctctcaaacactgatgccactggcttggaagagatcgatcgagcattgggaatcccagagcttgtaaatcaaggtcaggcgctggaacctaaacaggataacggaggtggaggctcacacgagaagttccccagcgacctggacctggacatgttcaacggcagcctggagtgcgacatggagagcatcatcagaagcgagctgatggacgccgacggcctggacttcaacttcgacagcctgggtggaggaggcagtgagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 126 - DNA, 534 bp, synthetic construct
  misc_feature 1..534: note = Tripartite activator - HSH2D FOXO3 NCOA3 (nt)
  source 1..534: mol_type = other DNA
tggttccacggcgccatcagcagagaggacgccgagaacctgctggagagccagcccctgggcagcttcctgatcagagtgagccacagccacgtgggctacaccctgagctacaaggcccagagcagctgctgccacttcatggtgaagctgctggacgacggcaccttcatgatccccggcgagaaggtggcccacaccagcctggacgccctggtgaccttccacaacggaggtggaggctcacacgagaagttccccagcgacctggacctggacatgttcaacggcagcctggagtgcgacatggagagcatcatcagaagcgagctgatggacgccgacggcctggacttcaacttcgacagcctgggtggaggaggcagtgagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 127 - DNA, 432 bp, synthetic construct
  misc_feature 1..432: note = Tripartite activator - FOXO3 FOXO3 NCOA3 (nt)
  source 1..432: mol_type = other DNA
cacgagaaattcccctccgatttggatctggatatgttcaacgggtcacttgaatgtgacatggagagcattatacgctccgaacttatggacgcagacggtcttgactttaactttgattccctcaacggaggtggaggctcacacgagaagttccccagcgacctggacctggacatgttcaacggcagcctggagtgcgacatggagagcatcatcagaagcgagctgatggacgccgacggcctggacttcaacttcgacagcctgggtggaggaggcagtgagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 128 - DNA, 447 bp, synthetic construct
  misc_feature 1..447: note = Tripartite activator - NCOA2 FOXO3 NCOA3 (nt)
  source 1..447: mol_type = other DNA
gaaagccccagcgacgagggcgctctgctggaccagctgtacctggccctgagaaacttcgacggcctggaggagatcgacagagccctgggcatccccgagctggtgagccagagccaggccgtggaccccgagcagttcaacggaggtggaggctcacacgagaagttccccagcgacctggacctggacatgttcaacggcagcctggagtgcgacatggagagcatcatcagaagcgagctgatggacgccgacggcctggacttcaacttcgacagcctgggtggaggaggcagtgagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 129 - DNA, 492 bp, synthetic construct
  misc_feature 1..492: note = Tripartite activator - ENL FOXO3 NCOA3 (nt)
  source 1..492: mol_type = other DNA
gcctacaccgacgagctggtggagctgcacagaagactgatggccctgagagagagaaacgtgctgcagcagatcgtgaacctgatcgaggagaccggccacttcaacgtgaccaacaccaccttcgacttcgacctgttcagcctggacgagaccaccgtgagaaagctgcagagctgcctggagaacggaggtggaggctcacacgagaagttccccagcgacctggacctggacatgttcaacggcagcctggagtgcgacatggagagcatcatcagaagcgagctgatggacgccgacggcctggacttcaacttcgacagcctgggtggaggaggcagtgagggccagagcgacgagagagccctgctggaccagctgcacaccctgctgagcaacaccgacgccaccggcctggaggagatcgacagagccctgggcatccccgagctggtgaaccagggccaggccctggagcccaagcaggac

SEQ ID NO: 130 - protein, 57 aa, synthetic construct
  REGION 1..57: note = PYGO1 transcriptional activation domain (aa)
PCGICTNEVNDDQDAILCEASCQKWFHRICTGMTETAYGLLTAEASAVWGCDTCMAD

SEQ ID NO: 131 - protein, 62 aa, synthetic construct
  REGION 1..62: note = ENL transcriptional activation domain (aa)
AYTDELVELHRRLMALRERNVLQQIVNLIEETGHFNVTNTTFDFDLFSLDETTVRKLQSCLE

SEQ ID NO: 132 - protein, 42 aa, synthetic construct
  REGION 1..42: note = FOXO3 transcriptional activation domain (aa)
HEKFPSDLDLDMFNGSLECDMESIIRSELMDADGLDFNFDSL

SEQ ID NO: 133 - protein, 49 aa, synthetic construct
  REGION 1..49: note = NCOA3 transcriptional activation domain (aa)
EGQSDERALLDQLHTLLSNTDATGLEEIDRALGIPELVNQGQALEPKQD

SEQ ID NO: 134 - protein, 76 aa, synthetic construct
  REGION 1..76: note = HSH2D transcriptional activation domain (aa)
WFHGAISREDAENLLESQPLGSFLIRVSHSHVGYTLSYKAQSSCCHFMVKLLDDGTFMIPGEKVAHTSLDALVTFH

SEQ ID NO: 135 - protein, 47 aa, synthetic construct
  REGION 1..47: note = NCOA2 transcriptional activation domain (aa)
ESPSDEGALLDQLYLALRNFDGLEEIDRALGIPELVSQSQAVDPEQF

SEQ ID NO: 136 - protein, 37 aa, synthetic construct
  REGION 1..37: note = NOTCH2 transcriptional activation domain (aa)
DIDECASNPCRKGATCINGVNGFRCICPEGPHHPSCY

SEQ ID NO: 137 - protein, 6 aa, synthetic construct
  REGION 1..6: note = Multipartite linker 1 (aa)
NGGGGS

SEQ ID NO: 138 - protein, 5 aa, synthetic construct
  REGION 1..5: note = Multipartite linker 2 (aa)
GGGGS

SEQ ID NO: 139 - protein, 15 aa, synthetic construct
  REGION 1..15: note = Multipartite linker 3 (aa)
GGGGSGGGGSGGGGS

SEQ ID NO: 140 - protein, 112 aa, synthetic construct
  REGION 1..112: note = Bipartite activator - PYGO1 NCOA3 (aa)
PCGICTNEVNDDQDAILCEASCQKWFHRICTGMTETAYGLLTAEASAVWGCDTCMADNGGGGSEGQSDERALLDQLHTLLSNTDATGLEEIDRALGIPELVNQGQALEPKQD

SEQ ID NO: 141 - protein, 92 aa, synthetic construct
  REGION 1..92: note = Bipartite activator - NOTCH2 NCOA3 (aa)
DIDECASNPCRKGATCINGVNGFRCICPEGPHHPSCYNGGGGSEGQSDERALLDQLHTLLSNTDATGLEEIDRALGIPELVNQGQALEPKQD

SEQ ID NO: 142 - protein, 103 aa, synthetic construct
  REGION 1..103: note = Bipartite activator - NCOA3 NCOA3 (aa)
EGQSDERALLDQLHTLLSNTDATGLEEIDRALGIPELVNQGQALEPKQDGGGGSEGQSDERALLDQLHTLLSNTDATGLEEIDRALGIPELVNQGQALEPKQD

SEQ ID NO: 143 - protein, 131 aa, synthetic construct
  REGION 1..131: note = Bipartite activator - HSH2D NCOA3 (aa)
WFHGAISREDAENLLESQPLGSFLIRVSHSHVGYTLSYKAQSSCCHFMVKLLDDGTFMIPGEKVAHTSLDALVTFHNGGGGSEGQSDERALLDQLHTLLSNTDATGLEEIDRALGIPELVNQGQALEPKQD

SEQ ID NO: 144 - protein, 96 aa, synthetic construct
  REGION 1..96: note = Bipartite activator - FOXO3 NCOA3 (aa)
HEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 145 - protein, 102 aa, synthetic construct
  REGION 1..102: note = Bipartite activator - NCOA2 NCOA3 (aa)
ESPSDEGALLDQLYLALRNFDGLEEIDRALGIPELVSQSQAVDPEQFNGGGGSEGQSDERALLDQLHTLLSNTDATGLEEIDRALGIPELVNQGQALEPKQD

SEQ ID NO: 146 - protein, 117 aa, synthetic construct
  REGION 1..117: note = Bipartite activator - ENL NCOA3 (aa)
AYTDELVELHRRLMALRERNVLQQIVNLIEETGHFNVTNTTFDFDLFSLDETTVRKLQSCLENGGGGSEGQSDERALLDQLHTLLSNTDATGLEEIDRALGIPELVNQGQALEPKQD

SEQ ID NO: 147 - protein, 105 aa, synthetic construct
  REGION 1..105: note = Bipartite activator - PYGO1 FOXO3 (aa)
PCGICTNEVNDDQDAILCEASCQKWFHRICTGMTETAYGLLTAEASAVWGCDTCMADNGGGGSHEKFPSDLDLDMFNGSLECDMESIIRSELMDADGLDFNFDSL

SEQ ID NO: 148 - protein, 85 aa, synthetic construct
  REGION 1..85: note = Bipartite activator - NOTCH2 FOXO3 (aa)
DIDECASNPCRKGATCINGVNGFRCICPEGPHHPSCYNGGGGSHEKFPSDLDLDMFNGSLECDMESIIRSELMDADGLDFNFDSL

SEQ ID NO: 149 - protein, 97 aa, synthetic construct
  REGION 1..97: note = Bipartite activator - NCOA3 FOXO3 (aa)
EGQSDERALLDQLHTLLSNTDATGLEEIDRALGIPELVNQGQALEPKQDNGGGGSHEKFPSDLDLDMFNGSLECDMESIIRSELMDADGLDFNFDSL

SEQ ID NO: 150 - protein, 124 aa, synthetic construct
  REGION 1..124: note = Bipartite activator - HSH2D FOXO3 (aa)
WFHGAISREDAENLLESQPLGSFLIRVSHSHVGYTLSYKAQSSCCHFMVKLLDDGTFMIPGEKVAHTSLDALVTFHNGGGGSHEKFPSDLDLDMFNGSLECDMESIIRSELMDADGLDFNFDSL

SEQ ID NO: 151 - protein, 90 aa, synthetic construct
  REGION 1..90: note = Bipartite activator - FOXO3 FOXO3 (aa)
HEKFPSDLDLDMFNGSLECDMESIIRSELMDADGLDFNFDSLNGGGGSHEKFPSDLDLDMFNGSLECDMESIIRSELMDADGLDFNFDSL

SEQ ID NO: 152 - protein, 95 aa, synthetic construct
  REGION 1..95: note = Bipartite activator - NCOA2 FOXO3 (aa)
ESPSDEGALLDQLYLALRNFDGLEEIDRALGIPELVSQSQAVDPEQFNGGGGSHEKFPSDLDLDMFNGSLECDMESIIRSELMDADGLDFNFDSL

SEQ ID NO: 153 - protein, 110 aa, synthetic construct
  REGION 1..110: note = Bipartite activator - ENL FOXO3 (aa)
AYTDELVELHRRLMALRERNVLQQIVNLIEETGHFNVTNTTFDFDLFSLDETTVRKLQSCLENGGGGSHEKFPSDLDLDMFNGSLECDMESIIRSELMDADGLDFNFDSL

SEQ ID NO: 154 - protein, 159 aa, synthetic construct
  REGION 1..159: note = Tripartite activator - PYGO1 FOXO3 NCOA3 (aa)
PCGICTNEVNDDQDAILCEASCQKWFHRICTGMTETAYGLLTAEASAVWGCDTCMADNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 155 - protein, 139 aa, synthetic construct
  REGION 1..139: note = Tripartite activator - NOTCH2 FOXO3 NCOA3 (aa)
DIDECASNPCRKGATCINGVNGFRCICPEGPHHPSCYNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 156 - protein, 151 aa, synthetic construct
  REGION 1..151: note = Tripartite activator - NCOA3 FOXO3 NCOA3 (aa)
EGQSDERALLDQLHTLLSNTDATGLEEIDRALGIPELVNQGQALEPKQDNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 157 - protein, 178 aa, synthetic construct
  REGION 1..178: note = Tripartite activator - HSH2D FOXO3 NCOA3 (aa)
WFHGAISREDAENLLESQPLGSFLIRVSHSHVGYTLSYKAQSSCCHFMVKLLDDGTFMIPGEKVAHTSLDALVTFHNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 158 - protein, 144 aa, synthetic construct
  REGION 1..144: note = Tripartite activator - FOXO3 FOXO3 NCOA3 (aa)
HEKFPSDLDLDMFNGSLECDMESIIRSELMDADGLDFNFDSLNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 159 - protein, 149 aa, synthetic construct
  REGION 1..149: note = Tripartite activator - NCOA2 FOXO3 NCOA3 (aa)
ESPSDEGALLDQLYLALRNFDGLEEIDRALGIPELVSQSQAVDPEQFNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 160 - protein, 164 aa, synthetic construct
  REGION 1..164: note = Tripartite activator - ENL FOXO3 NCOA3 (aa)
AYTDELVELHRRLMALRERNVLQQIVNLIEETGHFNVTNTTFDFDLFSLDETTVRKLQSCLENGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 161 - DNA, 147 bp, synthetic construct
  misc_feature 1..147: note = VP64 1 (nt)
  source 1..147: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatg

SEQ ID NO: 162 - protein, 49 aa, synthetic construct
  REGION 1..49: note = VP64 1 (aa)
DALDDFDLDMLGSDALDDFDLDMLGSDALDDFDLDMLGSDALDDFDLDM

SEQ ID NO: 163 - DNA, 150 bp, synthetic construct
  misc_feature 1..150: note = VP64 2 (nt)
  source 1..150: mol_type = other DNA
gacgcgctggacgatttcgatctcgacatgctgggttctgatgccctcgatgactttgacctggatatgttgggaagcgacgcattggatgactttgatctggacatgctcggctccgatgctctggacgatttcgatctcgatatgtta

SEQ ID NO: 164 - protein, 50 aa, synthetic construct
  REGION 1..50: note = VP64 2 (aa)
DALDDFDLDMLGSDALDDFDLDMLGSDALDDFDLDMLGSDALDDFDLDML

SEQ ID NO: 165 - DNA, 240 bp, synthetic construct
  misc_feature 1..240: note = Control peptide (nt)
  source 1..240: mol_type = other DNA
ggaattgtgaccctgcctcatcgctacgtgtacctgatcaggtttcttgcggaccacgtcgatgctagccaatccctaactacgagaactcgtattgacacattcatgtgcgtcgcctgctctttcgacggcagtaattataccgcagtgttaagtccccagatactcgccatccgacgaaactcttgtgtacgagatatgcgattcgttgctagacgtgagtacaccaaggccgtgttc

SEQ ID NO: 166 - DNA, 66 bp, synthetic construct
  misc_feature 1..66: note = FLAG epitope tag (nt)
  source 1..66: mol_type = other DNA
gactacaaagaccatgacggtgattataaagatcatgacatcgattacaaggatgacgatgacaag

SEQ ID NO: 167 - protein, 22 aa, synthetic construct
  REGION 1..22: note = FLAG epitope tag (aa)
DYKDHDGDYKDHDIDYKDDDDK

SEQ ID NO: 168 - DNA, 76 bp, synthetic construct
  misc_feature 1..76: note = SaCas9 gRNA scaffold DNA sequence
  source 1..76: mol_type = other DNA
gttttagtactctggaaacagaatctactaaaacaaggcaaaatgccgtgtttatctcgtcaacttgttggcgaga

SEQ ID NO: 169 - RNA, 76 nt, synthetic construct
  misc_feature 1..76: note = SaCas9 gRNA scaffold RNA sequence
  source 1..76: mol_type = other RNA
gttttagtactctggaaacagaatctactaaaacaaggcaaaatgccgtgtttatctcgtcaacttgttggcgaga

SEQ ID NO: 170 - DNA, 86 bp, synthetic construct
  misc_feature 1..86: note = SpCas9 gRNA scaffold DNA sequence
  source 1..86: mol_type = other DNA
gtttaagagctatgctggaaacagcatagcaagtttaaataaggctagtccgttatcaacttgaaaaagtggcaccgagtcggtgc

SEQ ID NO: 171 - RNA, 86 nt, synthetic construct
  misc_feature 1..86: note = SpCas9 gRNA scaffold RNA sequence
  source 1..86: mol_type = other RNA
gtttaagagctatgctggaaacagcatagcaagtttaaataaggctagtccgttatcaacttgaaaaagtggcaccgagtcggtgc

SEQ ID NO: 172 - DNA, 48 bp, synthetic construct
  misc_feature 1..48: note = linker (nt)
  source 1..48: mol_type = other DNA
aacccaaagaagaagcggaaggtcggtatccacggagtcccagcagcc

SEQ ID NO: 173 - protein, 16 aa, synthetic construct
  REGION 1..16: note = linker (aa)
NPKKKRKVGIHGVPAA

SEQ ID NO: 174 - protein, 16 aa, synthetic construct
  REGION 1..16: note = Linker (aa)
SGSETPGTSESATPES

SEQ ID NO: 175 - DNA, 96 bp, synthetic construct
  misc_feature 1..96: note = FXN promoter-targeting gRNA A - DNA
  source 1..96: mol_type = other DNA
tcacacagctaggaagtggggttttagtactctggaaacagaatctactaaaacaaggcaaaatgccgtgtttatctcgtcaacttgttggcgaga

SEQ ID NO: 176 - protein, 61 aa, synthetic construct
  REGION 1..61: note = DPOLA transcriptional activation domain (aa)
LERLKKAKAGEKYKYEVEDFTGVYEEVDEEQYSKLVQARQDDDWIVDDDGIGYVEDGREIF

SEQ ID NO: 177 - protein, 22 aa, synthetic construct
  REGION 1..22: note = PSA1 transcriptional activation domain (aa)
YDNDVTVWSPQGRIHQIEYAME

SEQ ID NO: 178 - protein, 70 aa, synthetic construct
  REGION 1..70: note = RBM39 transcriptional activation domain (aa)
LYVGSLHFNITEDMLRGIFEPFGRIESIQLMMDSETGRSKGYGFITFSDSECAKKALEQLNGFELAGRPM

SEQ ID NO: 179 - protein, 72 aa, synthetic construct
  REGION 1..72: note = HERC2 transcriptional activation domain (aa)
RKADLENHNKDGGFWTVIDGKVYDIKDFQTQSLTGNSILAQFAGEDPVVALEAALQFEDTRESMHAFCVGQY

SEQ ID NO: 180 - protein, 1179 aa, synthetic construct
  REGION 1..1179: note = Multipartite activator-dSaCas9 - FOXO3 NCOA3 (aa)
HEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 181 - protein, 1242 aa, synthetic construct
  REGION 1..1242: note = Multipartite activator-dSaCas9 - PYGO1 FOXO3 NCOA3 (aa)
PCGICTNEVNDDQDAILCEASCQKWFHRICTGMTETAYGLLTAEASAVWGCDTCMADNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 182 - protein, 1222 aa, synthetic construct
  REGION 1..1222: note = Multipartite activator-dSaCas9 - NOTCH2 FOXO3 NCOA3 (aa)
DIDECASNPCRKGATCINGVNGFRCICPEGPHHPSCYNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 183 - protein, 1234 aa, synthetic construct
  REGION 1..1234: note = Multipartite activator-dSaCas9 - NCOA3 FOXO3 NCOA3 (aa)
EGQSDERALLDQLHTLLSNTDATGLEEIDRALGIPELVNQGQALEPKQDNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 184 - protein, 1261 aa, synthetic construct
  REGION 1..1261: note = Multipartite activator-dSaCas9 - HSH2D FOXO3 NCOA3 (aa)
WFHGAISREDAENLLESQPLGSFLIRVSHSHVGYTLSYKAQSSCCHFMVKLLDDGTFMIPGEKVAHTSLDALVTFHNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 185 - protein, 1227 aa, synthetic construct
  REGION 1..1227: note = Multipartite activator-dSaCas9 - FOXO3 FOXO3 NCOA3 (aa)
HEKFPSDLDLDMFNGSLECDMESIIRSELMDADGLDFNFDSLNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 186 - protein, 1232 aa, synthetic construct
  REGION 1..1232: note = Multipartite activator-dSaCas9 - NCOA2 FOXO3 NCOA3 (aa)
ESPSDEGALLDQLYLALRNFDGLEEIDRALGIPELVSQSQAVDPEQFNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 187 - protein, 1247 aa, synthetic construct
  REGION 1..1247: note = Multipartite activator-dSaCas9 - ENL FOXO3 NCOA3 (aa)
AYTDELVELHRRLMALRERNVLQQIVNLIEETGHFNVTNTTFDFDLFSLDETTVRKLQSCLENGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 188 - DNA, 516 bp, synthetic construct
  misc_feature 1..516: note = NCOA3-NCOA3(codon optimized)-NCOA3 (nt)
  source 1..516: mol_type = other DNA
gagggccagagcgacgagagagccctgctggaccagctgcacaccctgctgagcaacaccgacgccaccggcctggaggagatcgacagagccctgggcatccccgagctggtgaaccagggccaggccctggagcccaagcaggacggtggaggaggcagtgaagggcaaagcgacgagcgagcactcttggatcagctccacacgctgctctcaaacactgatgccactggcttggaagagatcgatcgagcattgggaatcccagagcttgtaaatcaaggtcaggcgctggaacctaaacaggatggaggtggaggctcagagggccagagcgacgagagagccctgctggaccagctgcacaccctgctgagcaacaccgacgccaccggcctggaggagatcgacagagccctgggcatccccgagctggtgaaccagggccaggccctggagcccaagcaggacggaggaggaggttctgggggtggaggtagcggtggtggaggtagt

SEQ ID NO: 189 - protein, 172 aa, synthetic construct
  REGION 1..172: note = NCOA3-NCOA3(codon optimized)-NCOA3 (aa)
EGQSDERALLDQLHTLLSNTDATGLEEIDRALGIPELVNQGQALEPKQDGGGGSEGQSDERALLDQLHTLLSNTDATGLEEIDRALGIPELVNQGQALEPKQDGGGGSEGQSDERALLDQLHTLLSNTDATGLEEIDRALGIPELVNQGQALEPKQDGGGGSGGGGSGGGGS

SEQ ID NO: 190 - DNA, 21 bp, Mus musculus
  misc_feature 1..21: note = mouse Nrf2 gRNA A spacer
  source 1..21: mol_type = unassigned DNA
gcccgagggcggggcatggac

SEQ ID NO: 191 - DNA, 21 bp, Mus musculus
  misc_feature 1..21: note = mouse Nrf2 gRNA B spacer
  source 1..21: mol_type = unassigned DNA
gcgagaggaggatcaacagtg

SEQ ID NO: 192 - DNA, 22 bp, Mus musculus
  misc_feature 1..22: note = mouse Nrf2 gRNA C spacer
  source 1..22: mol_type = unassigned DNA
ggcagttggcctcttgcaaagt

SEQ ID NO: 193 - protein, 124 aa, synthetic construct
  REGION 1..124: note = p65AD (aa)
PTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 194 - protein, 190 aa, synthetic construct
  REGION 1..190: note = Rta domain (aa)
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 195 - protein, 124 aa, synthetic construct
  REGION 1..124: note = HSF1 domain (aa)
GFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 196 - protein, 522 aa, synthetic construct
  REGION 1..522: note = VPR (aa)
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 197 - protein, 378 aa, synthetic construct
  REGION 1..378: note = VPH (aa)
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 198 - protein, 718 aa, synthetic construct
  REGION 1..718: note = TET1 catalytic domain (aa)
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 199 - protein, 2404 aa, synthetic construct
  REGION 1..2404: note = CREB binding protein (CBP) (AA)
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQAILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 200 - protein, 2414 aa, synthetic construct
  REGION 1..2414: note = p300 (AA)
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 201 - protein, 617 aa, synthetic construct; identical to SEQ ID NO: 34
  REGION 1..617: note = p300 domain (AA)

SEQ ID NO: 202 - protein, 489 aa, synthetic construct
  REGION 1..489: note = HSF1 (AA)
MDLPVGPGAAGPSNVPAFLTKLWTLVSDPDTDALICWSPSGNSFHVFDQGQFAKEVLPKYFKHNNMASFVRQLNMYGFRKVVHIEQGGLVKPERDDTEFQHPCFLRGQEQLLENIKRKVTSVSTLKSEDIKIRQDSVTKLLTDVQLMKGKQECMDSKLLAMKHENEALWREVASLRQKHAQQQKVVNKLIQFLISLVQSNRILGVKRKIPLMLNDSGSAHSMPKYSRQFSLEHVHGSGPYSAPSPAYSSSSLYAPDAVASSGPIISDITELAPASPMASPGGSIDERPLSSSPLVRVKEEPPSPPQSPRVEEASPGRPSSVDTLLSPTALIDSILRESEPAPASVTALTDARGHTDTEGRPPSPPPTSTPEKCLSVACLDKNELSDHLDAMDSNLDNLQTMLSSHGFSVDTSALLDLFSPSVTVPDMSLPDLDSSLASIQELLSPQEPPRPPEAENSSPDSAGALHSAAAVPAGPRLRGHREQRPAGAV

SEQ ID NO: 203 - protein, 2136 aa, synthetic construct
  REGION 1..2136: note = TET1 (AA)
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTIQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 204 - protein, 27 aa, synthetic construct
  REGION 1..27: note = SunTag repeat V1
LLPKNYHLENEVARLKKLVGERGGSGG

SEQ ID NO: 205 - protein, 19 aa, synthetic construct
  REGION 1..19: note = SunTag GCN4 peptide V2 (AA)
EELLSKNYHLENEVARLKK

SEQ ID NO: 206 - protein, 5 aa, synthetic construct
  REGION 1..5: note = linker (SunTag)
GSGSG

SEQ ID NO: 207 - protein, 24 aa, synthetic construct
  REGION 1..24: note = SunTag repeat V4
EELLSKNYHLENEVARLKKGSGSG

SEQ ID NO: 208 - DNA, 20 bp, synthetic construct
  misc_feature 1..20: note = target site A or gRNA spacer sequence
  source 1..20: mol_type = other DNA
tcacacagctaggaagtggg

SEQ ID NO: 209 - DNA, 20 bp, synthetic construct
  misc_feature 1..20: note = target site B or gRNA spacer sequence
  source 1..20: mol_type = other DNA
gaggctgcttggccgccggt

SEQ ID NO: 210 - DNA, 20 bp, synthetic construct
  misc_feature 1..20: note = target site C or gRNA spacer sequence
  source 1..20: mol_type = other DNA
gcccgctccgccctccagcg

SEQ ID NO: 211 - DNA, 20 bp, synthetic construct
  misc_feature 1..20: note = target site D or gRNA spacer sequence
  source 1..20: mol_type = other DNA
agcctgctttgtgcaaagca

SEQ ID NO: 212 - DNA, 22 bp, synthetic construct
  misc_feature 1..22: note = target site E or gRNA spacer sequence
  source 1..22: mol_type = other DNA
ccggccgatgacgcgccgcggg

SEQ ID NO: 213 - DNA, 22 bp, synthetic construct
  misc_feature 1..22: note = target site F or gRNA spacer sequence
  source 1..22: mol_type = other DNA
ggccggctactgcgcggcgccc

SEQ ID NO: 214 - DNA, 22 bp, synthetic construct
  misc_feature 1..22: note = target site G or gRNA spacer sequence
  source 1..22: mol_type = other DNA
acctctagctgctcccccacag

SEQ ID NO: 215 - DNA, 22 bp, synthetic construct
  misc_feature 1..22: note = target site H or gRNA spacer sequence
  source 1..22: mol_type = other DNA
ccgcccgctccgccctccagcg

SEQ ID NO: 216 - DNA, 22 bp, synthetic construct
  misc_feature 1..22: note = target site I or gRNA spacer sequence
  source 1..22: mol_type = other DNA
gcccgagagtccacatgctgct

SEQ ID NO: 217 - DNA, 22 bp, synthetic construct
  misc_feature 1..22: note = target site J or gRNA spacer sequence
  source 1..22: mol_type = other DNA
gctctccatttttgttaaatgc

SEQ ID NO: 218 - DNA, 20 bp, synthetic construct
  misc_feature 1..20: note = target site K or gRNA spacer sequence
  source 1..20: mol_type = other DNA
ctgctgtaaacccataccgg

SEQ ID NO: 219 - DNA, 20 bp, synthetic construct
  misc_feature 1..20: note = target site L or gRNA spacer sequence
  source 1..20: mol_type = other DNA
gcagagtacagatttacaca

SEQ ID NO: 220 - DNA, 20 bp, synthetic construct
  misc_feature 1..20: note = target site M or gRNA spacer sequence
  source 1..20: mol_type = other DNA
caagggagactgcagcctgg

SEQ ID NO: 221 - DNA, 20 bp, synthetic construct
  misc_feature 1..20: note = target site N or gRNA spacer sequence
  source 1..20: mol_type = other DNA
aagctgggaagttcttcctg

SEQ ID NO: 222 - DNA, 20 bp, synthetic construct
  misc_feature 1..20: note = target site O or gRNA spacer sequence
  source 1..20: mol_type = other DNA
atgcacgaatagtgctaagc

SEQ ID NO: 223 - DNA, 20 bp, synthetic construct
  misc_feature 1..20: note = target site P or gRNA spacer sequence
  source 1..20: mol_type = other DNA
tacacaaggcatccgtctcc

SEQ ID NO: 224 - DNA, 20 bp, synthetic construct
  misc_feature 1..20: note = target site Q or gRNA spacer sequence
  source 1..20: mol_type = other DNA
ggctgcttggccgccggtat

SEQ ID NO: 225 - DNA, 20 bp, synthetic construct
  misc_feature 1..20: note = target site R or gRNA spacer sequence
  source 1..20: mol_type = other DNA
tttagaagcggcgggccacc

SEQ ID NO: 226 - DNA, 20 bp, synthetic construct
  misc_feature 1..20: note = target site S or gRNA spacer sequence
  source 1..20: mol_type = other DNA
aattgaggctgcttggccgc

SEQ ID NO: 227 - DNA, 20 bp, synthetic construct
  misc_feature 1..20: note = target site T or gRNA spacer sequence
  source 1..20: mol_type = other DNA
gcaaagcacggagtgcaacc

SEQ ID NO: 228 - DNA, 21 bp, synthetic construct
  misc_feature 1..21: note = target site U or gRNA spacer sequence
  source 1..21: mol_type = other DNA
tgcatgggacaaccaccacga

SEQ ID NO: 229 - RNA, 20 nt, synthetic construct
  misc_feature 1..20: note = gRNA A spacer RNA sequence
  source 1..20: mol_type = other RNA
tcacacagctaggaagtggg

SEQ ID NO: 230 - RNA, 20 nt, synthetic construct
  misc_feature 1..20: note = gRNA B spacer RNA sequence
  source 1..20: mol_type = other RNA
gaggctgcttggccgccggt

SEQ ID NO: 231 - RNA, 20 nt, synthetic construct
  misc_feature 1..20: note = gRNA C spacer RNA sequence
  source 1..20: mol_type = other RNA
gcccgctccgccctccagcg

SEQ ID NO: 232 - RNA, 20 nt, synthetic construct
  misc_feature 1..20: note = gRNA D spacer RNA sequence
  source 1..20: mol_type = other RNA
agcctgctttgtgcaaagca

SEQ ID NO: 233 - RNA, 22 nt, synthetic construct
  misc_feature 1..22: note = gRNA E spacer RNA sequence
  source 1..22: mol_type = other RNA
ccggccgatgacgcgccgcggg

SEQ ID NO: 234 - RNA, 22 nt, synthetic construct
  misc_feature 1..22: note = gRNA F spacer RNA sequence
  source 1..22: mol_type = other RNA
ggccggctactgcgcggcgccc

SEQ ID NO: 235 - RNA, 22 nt, synthetic construct
  misc_feature 1..22: note = gRNA G spacer RNA sequence
  source 1..22: mol_type = other RNA
acctctagctgctcccccacag

SEQ ID NO: 236 - RNA, 22 nt, synthetic construct
  misc_feature 1..22: note = gRNA H spacer RNA sequence
  source 1..22: mol_type = other RNA
ccgcccgctccgccctccagcg

SEQ ID NO: 237 - RNA, 22 nt, synthetic construct
  misc_feature 1..22: note = gRNA I spacer RNA sequence
  source 1..22: mol_type = other RNA
gcccgagagtccacatgctgct

SEQ ID NO: 238 - RNA, 22 nt, synthetic construct
  misc_feature 1..22: note = gRNA J spacer RNA sequence
  source 1..22: mol_type = other RNA
gctctccatttttgttaaatgc

SEQ ID NO: 239 - RNA, 20 nt, synthetic construct
  misc_feature 1..20: note = gRNA K spacer RNA sequence
  source 1..20: mol_type = other RNA
ctgctgtaaacccataccgg

SEQ ID NO: 240 - RNA, 20 nt, synthetic construct
  misc_feature 1..20: note = gRNA L spacer RNA sequence
  source 1..20: mol_type = other RNA
gcagagtacagatttacaca

SEQ ID NO: 241 - RNA, 20 nt, synthetic construct
  misc_feature 1..20: note = gRNA M spacer RNA sequence
  source 1..20: mol_type = other RNA
caagggagactgcagcctgg

SEQ ID NO: 242 - RNA, 20 nt, synthetic construct
  misc_feature 1..20: note = gRNA N spacer RNA sequence
  source 1..20: mol_type = other RNA
aagctgggaagttcttcctg

SEQ ID NO: 243 - RNA, 20 nt, synthetic construct
  misc_feature 1..20: note = gRNA O spacer RNA sequence
  source 1..20: mol_type = other RNA
atgcacgaatagtgctaagc

SEQ ID NO: 244 - RNA, 20 nt, synthetic construct
  misc_feature 1..20: note = gRNA P spacer RNA sequence
  source 1..20: mol_type = other RNA
tacacaaggcatccgtctcc

SEQ ID NO: 245 - RNA, 20 nt, synthetic construct
  misc_feature 1..20: note = gRNA Q spacer RNA sequence
  source 1..20: mol_type = other RNA
ggctgcttggccgccggtat

SEQ ID NO: 246 - RNA, 20 nt, synthetic construct
  misc_feature 1..20: note = gRNA R spacer RNA sequence
  source 1..20: mol_type = other RNA
tttagaagcggcgggccacc

SEQ ID NO: 247 - RNA, 20 nt, synthetic construct
  misc_feature 1..20: note = gRNA S spacer RNA sequence
  source 1..20: mol_type = other RNA
aattgaggctgcttggccgc

SEQ ID NO: 248 - RNA, 20 nt, synthetic construct
  misc_feature 1..20: note = gRNA T spacer RNA sequence
  source 1..20: mol_type = other RNA
gcaaagcacggagtgcaacc

SEQ ID NO: 249 - RNA, 21 nt, synthetic construct
  misc_feature 1..21: note = gRNA U spacer RNA sequence
  source 1..21: mol_type = other RNA
tgcatgggacaaccaccacga

SEQ ID NO: 250 - RNA, 96 nt, synthetic construct
  misc_feature 1..96: note = gRNA A
  source 1..96: mol_type = other RNA
tcacacagctaggaagtggggttttagtactctggaaacagaatctactaaaacaaggcaaaatgccgtgtttatctcgtcaacttgttggcgaga

SEQ ID NO: 251 - RNA, 96 nt, synthetic construct
  misc_feature 1..96: note = gRNA B
  source 1..96: mol_type = other RNA
gaggctgcttggccgccggtgttttagtactctggaaacagaatctactaaaacaaggcaaaatgccgtgtttatctcgtcaacttgttggcgaga

SEQ ID NO: 252 - RNA, 96 nt, synthetic construct
  misc_feature 1..96: note = gRNA C
  source 1..96: mol_type = other RNA
gcccgctccgccctccagcggttttagtactctggaaacagaatctactaaaacaaggcaaaatgccgtgtttatctcgtcaacttgttggcgaga

SEQ ID NO: 253 - RNA, 96 nt, synthetic construct
  misc_feature 1..96: note = gRNA D
  source 1..96: mol_type = other RNA
agcctgctttgtgcaaagcagttttagtactctggaaacagaatctactaaaacaaggcaaaatgccgtgtttatctcgtcaacttgttggcgaga

SEQ ID NO: 254 - RNA, 98 nt, synthetic construct
  misc_feature 1..98: note = gRNA E
  source 1..98: mol_type = other RNA
ccggccgatgacgcgccgcggggttttagtactctggaaacagaatctactaaaacaaggcaaaatgccgtgtttatctcgtcaacttgttggcgaga

SEQ ID NO: 255 - RNA, 98 nt, synthetic construct
  misc_feature 1..98: note = gRNA F
  source 1..98: mol_type = other RNA
ggccggctactgcgcggcgcccgttttagtactctggaaacagaatctactaaaacaaggcaaaatgccgtgtttatctcgtcaacttgttggcgaga

SEQ ID NO: 256 - RNA, 98 nt, synthetic construct
  misc_feature 1..98: note = gRNA G
  source 1..98: mol_type = other RNA
acctctagctgctcccccacaggttttagtactctggaaacagaatctactaaaacaaggcaaaatgccgtgtttatctcgtcaacttgttggcgaga

SEQ ID NO: 257 - RNA, 98 nt, synthetic construct
  misc_feature 1..98: note = gRNA H
  source 1..98: mol_type = other RNA
ccgcccgctccgccctccagcggttttagtactctggaaacagaatctactaaaacaaggcaaaatgccgtgtttatctcgtcaacttgttggcgaga

SEQ ID NO: 258 - RNA, 98 nt, synthetic construct
  misc_feature 1..98: note = gRNA I
  source 1..98: mol_type = other RNA
gcccgagagtccacatgctgctgttttagtactctggaaacagaatctactaaaacaaggcaaaatgccgtgtttatctcgtcaacttgttggcgaga

SEQ ID NO: 259 - RNA, 98 nt, synthetic construct
  misc_feature 1..98: note = gRNA J
  source 1..98: mol_type = other RNA
gctctccatttttgttaaatgcgttttagtactctggaaacagaatctactaaaacaaggcaaaatgccgtgtttatctcgtcaacttgttggcgaga

SEQ ID NO: 260 - RNA, 106 nt, synthetic construct
  misc_feature 1..106: note = gRNA K
  source 1..106: mol_type = other RNA
ctgctgtaaacccataccgggtttaagagctatgctggaaacagcatagcaagtttaaataaggctagtccgttatcaacttgaaaaagtggcaccgagtcggtgc

SEQ ID NO: 261 - RNA, 106 nt, synthetic construct
  misc_feature 1..106: note = gRNA L
  source 1..106: mol_type = other RNA
gcagagtacagatttacacagtttaagagctatgctggaaacagcatagcaagtttaaataaggctagtccgttatcaacttgaaaaagtggcaccgagtcggtgc

SEQ ID NO: 262 - RNA, 106 nt, synthetic construct
  misc_feature 1..106: note = gRNA M
  source 1..106: mol_type = other RNA
caagggagactgcagcctgggtttaagagctatgctggaaacagcatagcaagtttaaataaggctagtccgttatcaacttgaaaaagtggcaccgagtcggtgc

SEQ ID NO: 263 - RNA, 106 nt, synthetic construct
  misc_feature 1..106: note = gRNA N
  source 1..106: mol_type = other RNA
aagctgggaagttcttcctggtttaagagctatgctggaaacagcatagcaagtttaaataaggctagtccgttatcaacttgaaaaagtggcaccgagtcggtgc

SEQ ID NO: 264 - RNA, 106 nt, synthetic construct
  misc_feature 1..106: note = gRNA O
  source 1..106: mol_type = other RNA
atgcacgaatagtgctaagcgtttaagagctatgctggaaacagcatagcaagtttaaataaggctagtccgttatcaacttgaaaaagtggcaccgagtcggtgc

SEQ ID NO: 265 - RNA, 106 nt, synthetic construct
  misc_feature 1..106: note = gRNA P
  source 1..106: mol_type = other RNA
tacacaaggcatccgtctccgtttaagagctatgctggaaacagcatagcaagtttaaataaggctagtccgttatcaacttgaaaaagtggcaccgagtcggtgc

SEQ ID NO: 266 - RNA, 106 nt, synthetic construct
  misc_feature 1..106: note = gRNA Q
  source 1..106: mol_type = other RNA
ggctgcttggccgccggtatgtttaagagctatgctggaaacagcatagcaagtttaaataaggctagtccgttatcaacttgaaaaagtggcaccgagtcggtgc

SEQ ID NO: 267 - RNA, 106 nt, synthetic construct
  misc_feature 1..106: note = gRNA R
  source 1..106: mol_type = other RNA
tttagaagcggcgggccaccgtttaagagctatgctggaaacagcatagcaagtttaaataaggctagtccgttatcaacttgaaaaagtggcaccgagtcggtgc

SEQ ID NO: 268 - RNA, 106 nt, synthetic construct
  misc_feature 1..106: note = gRNA S
  source 1..106: mol_type = other RNA
aattgaggctgcttggccgcgtttaagagctatgctggaaacagcatagcaagtttaaataaggctagtccgttatcaacttgaaaaagtggcaccgagtcggtgc

SEQ ID NO: 269 - RNA, 106 nt, synthetic construct
  misc_feature 1..106: note = gRNA T
  source 1..106: mol_type = other RNA
gcaaagcacggagtgcaaccgtttaagagctatgctggaaacagcatagcaagtttaaataaggctagtccgttatcaacttgaaaaagtggcaccgagtcggtgc

SEQ ID NO: 270 - RNA, 97 nt, synthetic construct
  misc_feature 1..97: note = gRNA U
  source 1..97: mol_type = other RNA
tgcatgggacaaccaccacgagttttagtactctggaaacagaatctactaaaacaaggcaaaatgccgtgtttatctcgtcaacttgttggcgaga

SEQ ID NO: 271 - protein, 1224 aa, synthetic construct
  REGION 1..1224: note = Multipartite activator-dSaCas9 - FOXO3 NCOA3 (aa)
MDYKDHDGDYKDHDIDYKDDDDKHGPRHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKAS

SEQ ID NO: 272 - protein, 1287 aa, synthetic construct
  REGION 1..1287: note = Multipartite activator-dSaCas9 - PYGO1 FOXO3 NCOA3 (aa)
MDYKDHDGDYKDHDIDYKDDDDKHGPRPCGICTNEVNDDQDAILCEASCQKWFHRICTGMTETAYGLLTAEASAVWGCDTCMADNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKAS

SEQ ID NO: 273 - protein, 1267 aa, synthetic construct
  REGION 1..1267: note = Multipartite activator-dSaCas9 - NOTCH2 FOXO3 NCOA3 (aa)
MDYKDHDGDYKDHDIDYKDDDDKHGPRDIDECASNPCRKGATCINGVNGFRCICPEGPHHPSCYNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKAS

SEQ ID NO: 274 - protein, 1279 aa, synthetic construct
  REGION 1..1279: note = Multipartite activator-dSaCas9 - NCOA3 FOXO3 NCOA3 (aa)
MDYKDHDGDYKDHDIDYKDDDDKHGPREGQSDERALLDQLHTLLSNTDATGLEEIDRALGIPELVNQGQALEPKQDNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKAS

SEQ ID NO: 275 - protein, 1306 aa, synthetic construct
  REGION 1..1306: note = Multipartite activator-dSaCas9 - HSH2D FOXO3 NCOA3 (aa)
MDYKDHDGDYKDHDIDYKDDDDKHGPRWFHGAISREDAENLLESQPLGSFLIRVSHSHVGYTLSYKAQSSCCHFMVKLLDDGTFMIPGEKVAHTSLDALVTFHNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKAS

SEQ ID NO: 276 - protein, 1272 aa, synthetic construct
  REGION 1..1272: note = Multipartite activator-dSaCas9 - FOXO3 FOXO3 NCOA3 (aa)
MDYKDHDGDYKDHDIDYKDDDDKHGPRHEKFPSDLDLDMFNGSLECDMESIIRSELMDADGLDFNFDSLNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKAS

SEQ ID NO: 277 - protein, 1277 aa, synthetic construct
  REGION 1..1277: note = Multipartite activator-dSaCas9 - NCOA2 FOXO3 NCOA3 (aa)
MDYKDHDGDYKDHDIDYKDDDDKHGPRESPSDEGALLDQLYLALRNFDGLEEIDRALGIPELVSQSQAVDPEQFNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKAS

SEQ ID NO: 278 - protein, 1292 aa, synthetic construct
  REGION 1..1292: note = Multipartite activator-dSaCas9 - ENL FOXO3 NCOA3 (aa)
MDYKDHDGDYKDHDIDYKDDDDKHGPRAYTDELVELHRRLMALRERNVLQQIVNLIEETGHFNVTNTTFDFDLFSLDETTVRKLQSCLENGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGSNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKAS

SEQ ID NO: 279 - DNA, 600 bp, synthetic construct
  misc_feature 1..600: note = Puromycin resistance gene (nt)
  source 1..600: mol_type = other DNA
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtgtcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 280 - protein, 199 aa, synthetic construct
  REGION 1..199: note = Puromycin resistance gene (aa)
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 281 - protein, 127 aa, synthetic construct
  REGION 1..127: note = Bipartite activator - PYGO1 NCOA3 (aa)
PCGICTNEVNDDQDAILCEASCQKWFHRICTGMTETAYGLLTAEASAVWGCDTCMADNGGGGSEGQSDERALLDQLHTLLSNTDATGLEEIDRALGIPELVNQGQALEPKQDGGGGSGGGGSGGGGS

SEQ ID NO: 282 - protein, 107 aa, synthetic construct
  REGION 1..107: note = Bipartite activator - NOTCH2 NCOA3 (aa)
DIDECASNPCRKGATCINGVNGFRCICPEGPHHPSCYNGGGGSEGQSDERALLDQLHTLLSNTDATGLEEIDRALGIPELVNQGQALEPKQDGGGGSGGGGSGGGGS

SEQ ID NO: 283 - protein, 118 aa, synthetic construct
  REGION 1..118: note = Bipartite activator - NCOA3 NCOA3 (aa)
EGQSDERALLDQLHTLLSNTDATGLEEIDRALGIPELVNQGQALEPKQDGGGGSEGQSDERALLDQLHTLLSNTDATGLEEIDRALGIPELVNQGQALEPKQDGGGGSGGGGSGGGGS

SEQ ID NO: 284 - protein, 146 aa, synthetic construct
  REGION 1..146: note = Bipartite activator - HSH2D NCOA3 (aa)
WFHGAISREDAENLLESQPLGSFLIRVSHSHVGYTLSYKAQSSCCHFMVKLLDDGTFMIPGEKVAHTSLDALVTFHNGGGGSEGQSDERALLDQLHTLLSNTDATGLEEIDRALGIPELVNQGQALEPKQDGGGGSGGGGSGGGGS

SEQ ID NO: 285 - protein, 111 aa, synthetic construct
  REGION 1..111: note = Bipartite activator - FOXO3 NCOA3 (aa)
HEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGS

SEQ ID NO: 286 - protein, 117 aa, synthetic construct
  REGION 1..117: note = Bipartite activator - NCOA2 NCOA3 (aa)
ESPSDEGALLDQLYLALRNFDGLEEIDRALGIPELVSQSQAVDPEQFNGGGGSEGQSDERALLDQLHTLLSNTDATGLEEIDRALGIPELVNQGQALEPKQDGGGGSGGGGSGGGGS

SEQ ID NO: 287 - protein, 132 aa, synthetic construct
  REGION 1..132: note = Bipartite activator - ENL NCOA3 (aa)
AYTDELVELHRRLMALRERNVLQQIVNLIEETGHFNVTNTTFDFDLFSLDETTVRKLQSCLENGGGGSEGQSDERALLDQLHTLLSNTDATGLEEIDRALGIPELVNQGQALEPKQDGGGGSGGGGSGGGGS

SEQ ID NO: 288 - protein, 120 aa, synthetic construct
  REGION 1..120: note = Bipartite activator - PYGO1 FOXO3 (aa)
PCGICTNEVNDDQDAILCEASCQKWFHRICTGMTETAYGLLTAEASAVWGCDTCMADNGGGGSHEKFPSDLDLDMFNGSLECDMESIIRSELMDADGLDFNFDSLGGGGSGGGGSGGGGS

SEQ ID NO: 289 - protein, 100 aa, synthetic construct
  REGION 1..100: note = Bipartite activator - NOTCH2 FOXO3 (aa)
DIDECASNPCRKGATCINGVNGFRCICPEGPHHPSCYNGGGGSHEKFPSDLDLDMFNGSLECDMESIIRSELMDADGLDFNFDSLGGGGSGGGGSGGGGS

SEQ ID NO: 290 - protein, 112 aa, synthetic construct
  REGION 1..112: note = Bipartite activator - NCOA3 FOXO3 (aa)
EGQSDERALLDQLHTLLSNTDATGLEEIDRALGIPELVNQGQALEPKQDNGGGGSHEKFPSDLDLDMFNGSLECDMESIIRSELMDADGLDFNFDSLGGGGSGGGGSGGGGS

SEQ ID NO: 291 - protein, 139 aa, synthetic construct
  REGION 1..139: note = Bipartite activator - HSH2D FOXO3 (aa)
WFHGAISREDAENLLESQPLGSFLIRVSHSHVGYTLSYKAQSSCCHFMVKLLDDGTFMIPGEKVAHTSLDALVTFHNGGGGSHEKFPSDLDLDMFNGSLECDMESIIRSELMDADGLDFNFDSLGGGGSGGGGSGGGGS

SEQ ID NO: 292 - protein, 105 aa, synthetic construct
  REGION 1..105: note = Bipartite activator - FOXO3 FOXO3 (aa)
HEKFPSDLDLDMFNGSLECDMESIIRSELMDADGLDFNFDSLNGGGGSHEKFPSDLDLDMFNGSLECDMESIIRSELMDADGLDFNFDSLGGGGSGGGGSGGGGS

SEQ ID NO: 293 - protein, 110 aa, synthetic construct
  REGION 1..110: note = Bipartite activator - NCOA2 FOXO3 (aa)
ESPSDEGALLDQLYLALRNFDGLEEIDRALGIPELVSQSQAVDPEQFNGGGGSHEKFPSDLDLDMFNGSLECDMESIIRSELMDADGLDFNFDSLGGGGSGGGGSGGGGS

SEQ ID NO: 294 - protein, 125 aa, synthetic construct
  REGION 1..125: note = Bipartite activator - ENL FOXO3 (aa)
AYTDELVELHRRLMALRERNVLQQIVNLIEETGHFNVTNTTFDFDLFSLDETTVRKLQSCLENGGGGSHEKFPSDLDLDMFNGSLECDMESIIRSELMDADGLDFNFDSLGGGGSGGGGSGGGGS

SEQ ID NO: 295 - protein, 174 aa, synthetic construct
  REGION 1..174: note = Tripartite activator - PYGO1 FOXO3 NCOA3 (aa)
PCGICTNEVNDDQDAILCEASCQKWFHRICTGMTETAYGLLTAEASAVWGCDTCMADNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGS

SEQ ID NO: 296 - protein, 154 aa, synthetic construct
  REGION 1..154: note = Tripartite activator - NOTCH2 FOXO3 NCOA3 (aa)
DIDECASNPCRKGATCINGVNGFRCICPEGPHHPSCYNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGS

SEQ ID NO: 297 - protein, 166 aa, synthetic construct
  REGION 1..166: note = Tripartite activator - NCOA3 FOXO3 NCOA3 (aa)
EGQSDERALLDQLHTLLSNTDATGLEEIDRALGIPELVNQGQALEPKQDNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGS

SEQ ID NO: 298 - protein, 193 aa, synthetic construct
  REGION 1..193: note = Tripartite activator - HSH2D FOXO3 NCOA3 (aa)
WFHGAISREDAENLLESQPLGSFLIRVSHSHVGYTLSYKAQSSCCHFMVKLLDDGTFMIPGEKVAHTSLDALVTFHNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGS

SEQ ID NO: 299 - protein, 159 aa, synthetic construct
  REGION 1..159: note = Tripartite activator - FOXO3 FOXO3 NCOA3 (aa)
HEKFPSDLDLDMFNGSLECDMESIIRSELMDADGLDFNFDSLNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGS

SEQ ID NO: 300 - protein, 164 aa, synthetic construct
  REGION 1..164: note = Tripartite activator - NCOA2 FOXO3 NCOA3 (aa)
ESPSDEGALLDQLYLALRNFDGLEEIDRALGIPELVSQSQAVDPEQFNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGS

SEQ ID NO: 301 - protein, 179 aa, synthetic construct
  REGION 1..179: note = Tripartite activator - ENL FOXO3 NCOA3 (aa)
AYTDELVELHRRLMALRERNVLQQIVNLIEETGHFNVTNTTFDFDLFSLDETTVRKLQSCLENGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQDGGGGSGGGGSGGGGS

SEQ ID NO: 302 - skipped

SEQ ID NO: 303 - protein, 528 aa, synthetic construct
  REGION 1..528: note = CasMini Cas12f/D326A/D510A/D143R/T147R/K330R/E528R (aa)
AKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 304-308 - skipped (5 entries)

SEQ ID NO: 309 - protein, 1053 aa, synthetic construct
  REGION 1..1053: note = dSaCas9 (aa)
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 310 - protein, 1368 aa, synthetic construct
  REGION 1..1368: note = dSpCas9 (aa)
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 311 - RNA, 29 nt, synthetic construct
  misc_feature 1..29: note = dAsCas12a gRNA scaffold
  source 1..29: mol_type = other RNA
gaaacaccgtaatttctactcttgtagat

SEQ ID NO: 312 - RNA, 25 nt, synthetic construct
  misc_feature 1..25: note = dCasPhi-2 gRNA scaffold
  source 1..25: mol_type = other RNA
caacgattgcccctcacgaggggac

SEQ ID NO: 313 - RNA, 90 nt, synthetic construct
  misc_feature 1..90: note = Un1Cas12f1 gRNA scaffold
  source 1..90: mol_type = other RNA
accgcttcacttagagtgaaggtgggctgcttgcatcagcctaatgtcgagaagtgctttcttcggaaagtaaccctcgaaacaaagaaa

SEQ ID NO: 314-315 - skipped (2 entries)

SEQ ID NO: 316 - RNA, 73 nt, synthetic construct
  misc_feature 1..73: note = CjCas9 gRNA scaffold
  source 1..73: mol_type = other RNA
gcggttttaggggattgtaaccccgcagagtcccgcaaactctttattttagtcccttttcagggactaaaac

SEQ ID NO: 317 - RNA, 235 nt, synthetic construct
  misc_feature 1..235: note = Cas12k gRNA scaffold
  source 1..235: mol_type = other RNA
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaag

SEQ ID NO: 318 - RNA, 160 nt, synthetic construct
  misc_feature 1..160: note = CasMini gRNA scaffold
  source 1..160: mol_type = other RNA
gggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 319 - DNA, 20 bp, synthetic construct
  misc_feature 1..20: note = gRNA V spacer
  source 1..20: mol_type = other DNA
aagtaaggcaaaaagattgc

SEQ ID NO: 320 - DNA, 20 bp, synthetic construct
  misc_feature 1..20: note = gRNA W spacer
  source 1..20: mol_type = other DNA
caattttactccttagggga

SEQ ID NO: 321 - DNA, 20 bp, synthetic construct
  misc_feature 1..20: note = gRNA X spacer
  source 1..20: mol_type = other DNA
gagagcagatactactgcca

SEQ ID NO: 322 - DNA, 20 bp, synthetic construct
  misc_feature 1..20: note = gRNA Y spacer
  source 1..20: mol_type = other DNA
tggagaccaagaatgctgct

SEQ ID NO: 323 - DNA, 20 bp, synthetic construct
  misc_feature 1..20: note = gRNA Z spacer
  source 1..20: mol_type = other DNA
tgattttgtgatcttgcgaa

SEQ ID NO: 324 - DNA, 20 bp, synthetic construct
  misc_feature 1..20: note = gRNA AA spacer
  source 1..20: mol_type = other DNA
taacatgctgtacaggttta

SEQ ID NO: 325 - DNA, 20 bp, synthetic construct
  misc_feature 1..20: note = gRNA AB spacer
  source 1..20: mol_type = other DNA
ctagaccatctagctttgtg

SEQ ID NO: 326 - DNA, 20 bp, synthetic construct
  misc_feature 1..20: note = gRNA AC spacer
  source 1..20: mol_type = other DNA
tctccacccactagatggca

SEQ ID NO: 327 - protein, 1307 aa, synthetic construct
  REGION 1..1307: note = dAsCas12a (dAsCpf1) (aa)
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 328 - protein, 1306 aa, synthetic construct
  REGION 1..1306: note = dAsCas12a (dAsCpf1) (aa)
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 329 - protein, 1307 aa, synthetic construct
  REGION 1..1307: note = WT AsCas12a (AsCpf1) (aa)
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 330 - protein, 1306 aa, synthetic construct
  REGION 1..1306: note = WT AsCas12a (AsCpf1) (aa)
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 331 - protein, 776 aa, synthetic construct
  REGION 1..776: note = dCasPhi-2 (aa)
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIALGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTSGSGPKKKRKVEDPKKKRKV

SEQ ID NO: 332 - protein, 775 aa, synthetic construct
  REGION 1..775: note = dCasPhi-2 (aa)
PKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIALGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTSGSGPKKKRKVEDPKKKRKV

SEQ ID NO: 333 - protein, 757 aa, synthetic construct
  REGION 1..757: note = WT CasPhi-2 (aa)
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 334 - protein, 756 aa, synthetic construct
  REGION 1..756: note = WT CasPhi-2 (aa)
PKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 335 - protein, 529 aa, synthetic construct
  REGION 1..529: note = WT Un1Cas12f1 (aa)
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 336 - protein, 528 aa, synthetic construct
  REGION 1..528: note = WT Un1Cas12f1 (aa)
AKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 337 - protein, 529 aa, synthetic construct
  REGION 1..529: note = dUn1Cas12f1 (aa)
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 338 - protein, 528 aa, synthetic construct
  REGION 1..528: note = dUn1Cas12f1 (aa)
AKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 339 - protein, 984 aa, synthetic construct
  REGION 1..984: note = dCjCas9 (aa)
MARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDAIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 340 - protein, 983 aa, synthetic construct
  REGION 1..983: note = dCjCas9 (aa)
ARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDAIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 341 - protein, 836 aa, synthetic construct
  REGION 1..836: note = WT CjCas9 (aa)
MSSGSGHMARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELGGGSGGYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 342 - protein, 835 aa, synthetic construct
  REGION 1..835: note = WT CjCas9 (aa)
SSGSGHMARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELGGGSGGYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 343 - protein, 663 aa, synthetic construct
  REGION 1..663: note = WT Cas12k (aa)
MSSTSGKNPTNPIVRTISCNLSAKEDVLRKVWEEMSQKNTPLIVQLLKSVSEQPEFEANKENGTITKKEITELRRDITEDSDLKKQSGRLRSSADSLVTEVYSSWLRLYQVRKNKKEGKEYFLNNILKSDVELVEQSNCDLQTIRCKAKEILSQVEEFIEQVNNKPKINKTTSAKKKINKSNKNNKAIEEILNRFFIGNIDKTLTNTLYEIHRKSPDILTQCAVAYLIKNDNKVSEAEENLTQLNKRSSAKEIEIKRLETQIQNTRLPNGRDITGEKYSQAFEKLVNQVPQDNEEFAEWIAILLKKVSSLPYPILYSSGDLSWYKDEKGNIFVYFNGWAEYHFQICCDKRQLRFFERFLKYYKALKASEKGEEKLSGSLVTLRSAHLLWRQGKGKGEPWKVNKLALHCTYDARLWTAEGTEEVRQEKTDKAQAEVNQAESNENIDSKQQKKLTKNKSSLSRLKNSFARPSKPLYRGQSNIIVGISFHPVELATLVVVDINTKEILICKTVKELLGDAFPLLSRRRRQQVHFRKEREKAQKKDSPCDLGESKLGEYVDRLLAKRIVEVAKEYQASCIVLPGLKGIREIRTSVIQAKAETKFPGDINAQELYVKEYNRQIHNWSYSRLQESIKSRAAELKISIKFGKQPSHSTLQEQAINLALSA

SEQ ID NO: 344 - protein, 662 aa, synthetic construct
  REGION 1..662: note = WT Cas12k (aa)
SSTSGKNPTNPIVRTISCNLSAKEDVLRKVWEEMSQKNTPLIVQLLKSVSEQPEFEANKENGTITKKEITELRRDITEDSDLKKQSGRLRSSADSLVTEVYSSWLRLYQVRKNKKEGKEYFLNNILKSDVELVEQSNCDLQTIRCKAKEILSQVEEFIEQVNNKPKINKTTSAKKKINKSNKNNKAIEEILNRFFIGNIDKTLTNTLYEIHRKSPDILTQCAVAYLIKNDNKVSEAEENLTQLNKRSSAKEIEIKRLETQIQNTRLPNGRDITGEKYSQAFEKLVNQVPQDNEEFAEWIAILLKKVSSLPYPILYSSGDLSWYKDEKGNIFVYFNGWAEYHFQICCDKRQLRFFERFLKYYKALKASEKGEEKLSGSLVTLRSAHLLWRQGKGKGEPWKVNKLALHCTYDARLWTAEGTEEVRQEKTDKAQAEVNQAESNENIDSKQQKKLTKNKSSLSRLKNSFARPSKPLYRGQSNIIVGISFHPVELATLVVVDINTKEILICKTVKELLGDAFPLLSRRRRQQVHFRKEREKAQKKDSPCDLGESKLGEYVDRLLAKRIVEVAKEYQASCIVLPGLKGIREIRTSVIQAKAETKFPGDINAQELYVKEYNRQIHNWSYSRLQESIKSRAAELKISIKFGKQPSHSTLQEQAINLALSA

SEQ ID NO: 345 - protein, 558 aa, synthetic construct
  REGION 1..558: note = CasMini
MGPKKKRKVGSGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERPAKRPAATKKAGQAKKKK

SEQ ID NO: 346 - protein, 557 aa, synthetic construct
  REGION 1..557: note = CasMini
GPKKKRKVGSGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERPAKRPAATKKAGQAKKKK

SEQ ID NO: 347 - DNA, 23 bp, synthetic construct
  misc_feature 1..23: note = Cas12a gRNA 1 spacer
  source 1..23: mol_type = other DNA
tggaagtaaggcaaaaagattgc

SEQ ID NO: 348 - DNA, 23 bp, synthetic construct
  misc_feature 1..23: note = Cas12a gRNA 2 spacer
  source 1..23: mol_type = other DNA
aggcaaaaagattgctttgaata

SEQ ID NO: 349 - DNA, 23 bp, synthetic construct
  misc_feature 1..23: note = Cas12a gRNA 3 spacer
  source 1..23: mol_type = other DNA
agtggttctcaaccaggggcaat

SEQ ID NO: 350 - DNA, 23 bp, synthetic construct
  misc_feature 1..23: note = Cas12a gRNA 4 spacer
  source 1..23: mol_type = other DNA
caattttactccttaggggacct

SEQ ID NO: 351 - DNA, 23 bp, synthetic construct
  misc_feature 1..23: note = Cas12a gRNA 5 spacer
  source 1..23: mol_type = other DNA
ttttcacaatgtctggagacatt

SEQ ID NO: 352 - DNA, 23 bp, synthetic construct
  misc_feature 1..23: note = Cas12a gRNA 6 spacer
  source 1..23: mol_type = other DNA
tgggaaatgcatcattaggtgat

SEQ ID NO: 353 - DNA, 23 bp, synthetic construct
  misc_feature 1..23: note = Cas12a gRNA 7 spacer
  source 1..23: mol_type = other DNA
aattgtactacagtagttaagta

SEQ ID NO: 354 - DNA, 23 bp, synthetic construct
  misc_feature 1..23: note = Cas12a gRNA 8 spacer
  source 1..23: mol_type = other DNA
agtatggtaacatgctgtacagg

SEQ ID NO: 355 - DNA, 23 bp, synthetic construct
  misc_feature 1..23: note = Cas12a gRNA 9 spacer
  source 1..23: mol_type = other DNA
tatagtaggctagaccatctagc

SEQ ID NO: 356 - DNA, 23 bp, synthetic construct
  misc_feature 1..23: note = Cas12a gRNA 10 spacer
  source 1..23: mol_type = other DNA
tcacaaaatcacctaatgatgca

SEQ ID NO: 357 - DNA, 23 bp, synthetic construct
  misc_feature 1..23: note = Cas12a gRNA 11 spacer
  source 1..23: mol_type = other DNA
ttgtcccatgcattgtaaggtgg

SEQ ID NO: 358 - DNA, 23 bp, synthetic construct
  misc_feature 1..23: note = Cas12a gRNA 12 spacer
  source 1..23: mol_type = other DNA
gagaaccactattctggtctaac

SEQ ID NO: 359 - DNA, 23 bp, synthetic construct
  misc_feature 1..23: note = Cas12a gRNA 13 spacer
  source 1..23: mol_type = other DNA
aattaaatattcaaagcaatctt

SEQ ID NO: 360 - DNA, 23 bp, synthetic construct
  misc_feature 1..23: note = Cas12a gRNA 14 spacer
  source 1..23: mol_type = other DNA
atctttttgccttacttccaagt

SEQ ID NO: 361 - DNA, 23 bp, synthetic construct
  misc_feature 1..23: note = Cas12a gRNA 15 spacer
  source 1..23: mol_type = other DNA
cttacttccaagttttagataat

SEQ ID NO: 362 - DNA, 23 bp, synthetic construct
  misc_feature 1..23: note = Cas12a gRNA 16 spacer
  source 1..23: mol_type = other DNA
gctcacgcctgtcgtctcagcac

SEQ ID NO: 363 - DNA, 23 bp, synthetic construct
  misc_feature 1..23: note = Cas12a gRNA 17 spacer
  source 1..23: mol_type = other DNA
gtgaggcccaggcgggcagatcg

SEQ ID NO: 364 - DNA, 23 bp, synthetic construct
  misc_feature 1..23: note = Cas12a gRNA 18 spacer
  source 1..23: mol_type = other DNA
cagcagctcccaagttcctcctg

SEQ ID NO: 365 - DNA, 23 bp, synthetic construct
  misc_feature 1..23: note = Cas12a gRNA 19 spacer
  source 1..23: mol_type = other DNA
cccaagttcctcctgtttagaat

SEQ ID NO: 366 - DNA, 23 bp, synthetic construct
  misc_feature 1..23: note = Cas12a gRNA 20 spacer
  source 1..23: mol_type = other DNA
taatattttcaaggctggatttt

SEQ ID NO: 367 - DNA, 23 bp, synthetic construct
  misc_feature 1..23: note = Cas12a gRNA 21 spacer
  source 1..23: mol_type = other DNA
ctggatttttttgaacgaaatgc

SEQ ID NO: 368 - DNA, 23 bp, synthetic construct
  misc_feature 1..23: note = Cas12a gRNA 22 spacer
  source 1..23: mol_type = other DNA
tagctattctgcagctctgaaag

SEQ ID NO: 369 - DNA, 23 bp, synthetic construct
  misc_feature 1..23: note = Cas12a gRNA 23 spacer
  source 1..23: mol_type = other DNA
tttcacctcgttccaggaaagca

SEQ ID NO: 370 - DNA, 23 bp, synthetic construct
  misc_feature 1..23: note = Cas12a gRNA 24 spacer
  source 1..23: mol_type = other DNA
tcactgcaagctctgcctcccgg

SEQ ID NO: 371 - DNA, 23 bp, synthetic construct
  misc_feature 1..23: note = Cas12a gRNA 25 spacer
  source 1..23: mol_type = other DNA
ccgccaccatgcctggctaattt

SEQ ID NO: 372 - DNA, 23 bp, synthetic construct
  misc_feature 1..23: note = Cas12a gRNA 26 spacer
  source 1..23: mol_type = other DNA
acacccagctagtttttgtattt

SEQ ID NO: 373 - DNA, 23 bp, synthetic construct
  misc_feature 1..23: note = Cas12a gRNA 27 spacer
  source 1..23: mol_type = other DNA
ttgtattttttgtagaaaggggg

SEQ ID NO: 374 - DNA, 20 bp, synthetic construct
  misc_feature 1..20: note = gRNA AD spacer
  source 1..20: mol_type = other DNA
tcccctaaggagtaaaattg

SEQ ID NO: 375 - DNA, 20 bp, synthetic construct
  misc_feature 1..20: note = gRNA AE spacer
  source 1..20: mol_type = other DNA
tctaactttgaattaaatat

SEQ ID NO: 376 - protein, 16 aa, synthetic construct
  REGION 1..16: note = Linker (AA)
SGSETPGTSESATPES

SEQ ID NO: 377 - protein, 144 aa, synthetic construct
  REGION 1..144: note = Tripartite activator - NCOA3, FOXO3, FOXO3 (AA)
EGQSDERALLDQLHTLLSNTDATGLEEIDRALGIPELVNQGQALEPKQDGGGGSHEKFPSDLDLDMFNGSLECDMESIIRSELMDADGLDFNFDSLNGGGGSHEKFPSDLDLDMFNGSLECDMESIIRSELMDADGLDFNFDSL

SEQ ID NO: 378 - protein, 80 aa, synthetic construct
  REGION 1..80: note = ZNF473 (AA)
AEEFVTLKDVGMDFTLGDWEQLGLEQGDTFWDTALDNCQDLFLLDPPRPNLTSHPDGSEDLEPLAGGSPEATSPDVTETK

SEQ ID NO: 379 - protein, 80 aa, synthetic construct
  REGION 1..80: note = ANM2 (AA)
ECSEAGLLQEGVQPEEFVAIADYAATDETQLSFLRGEKILILRQTTADWWWGERAGCCGYIPANHVGKHVDEYDPEDTWQ

SEQ ID NO: 380 - protein, 80 aa, synthetic construct
  REGION 1..80: note = KIBRA (AA)
PRPELPLPEGWEEARDFDGKVYYIDHTNRTTSWIDPRDRYTKPLTFADCISDELPLGWEEAYDPQVGDYFIDHNTKTTQI

SEQ ID NO: 381 - protein, 80 aa, synthetic construct
  REGION 1..80: note = NOTCH2 (NOTC2) (AA)
EACNSHACQWDGGDCSLTMENPWANCSSPLPCWDYINNQCDELCNTVECLFDNFECQGNSKTCKYDKYCADHFKDNHCDQ

SEQ ID NO: 382 - protein, 80 aa, synthetic construct
  REGION 1..80: note = IKKA (AA)
LVGSSLEGAVTPQTSAWLPPTSAEHDHSLSCVVTPQDGETSAQMIEENLNCLGHLSTIIHEANEEQGNSMMNLDWSWLTE

SEQ ID NO: 383 - protein, 80 aa, synthetic construct
  REGION 1..80: note = APBB1 (AA)
GSPSYGSPEDTDSFWNPNAFETDSDLPAGWMRVQDTSGTYYWHIPTGTTQWEPPGRASPSQGSSPQEESQLTWTGFAHGE

SEQ ID NO: 384 - protein, 80 aa, synthetic construct
  REGION 1..80: note = SMN2 (AA)
GNREEQNLSDLLSPICEVANNIEQNAQENENESQVSTDESENSRSPGNKSDNIKPKSAPWNSFLPPPPPMPGPRLGPGKP

SEQ ID NO: 385 - protein, 80 aa, synthetic construct
  REGION 1..80: note = SERTAD2 (AA)
LMDSLPGNFEITTSTGFLTDLTLDDILFADIDTSMYDFDPCTSSSGTASKMAPVSADDLLKTLAPYSSQPVTPSQPFKMD

SEQ ID NO: 386 - protein, 80 aa, synthetic construct
  REGION 1..80: note = MYBA (AA)
FYIPVQIPGYQYVSPEGNCIEHVQPTSAFIQQPFIDEDPDKEKKIKELEMLLMSAENEVRRKRIPSQPGSFSSWSGSFLM

SEQ ID NO: 387 - protein, 871 aa, synthetic construct
  REGION 1..871: note = ZNF473 - Human, Full-length (AA)
MAEEFVTLKDVGMDFTLGDWEQLGLEQGDTFWDTALDNCQDLFLLDPPRPNLTSHPDGSEDLEPLAGGSPEATSPDVTETKNSPLMEDFFEEGFSQEIIEMLSKDGFWNSNFGEACIEDTWLDSLLGDPESLLRSDIATNGESPTECKSHELKRGLSPVSTVSTGEDSMVHNVSEKTLTPAKSKEYRGEFFSYSDHSQQDSVQEGEKPYQCSECGKSFSGSYRLTQHWITHTREKPTVHQECEQGFDRNASLSVYPKTHTGYKFYVCNEYGTTFSQSTYLWHQKTHTGEKPCKSQDSDHPPSHDTQPGEHQKTHTDSKSYNCNECGKAFTRIFHLTRHQKIHTRKRYECSKCQATFNLRKHLIQHQKTHAAKTTSECQECGKIFRHSSLLIEHQALHAGEEPYKCNERGKSFRHNSTLKIHQRVHSGEKPYKCSECGKAFHRHTHLNEHRRIHTGYRPHKCQECVRSFSRPSHLMRHQAIHTAEKPYSCAECKETFSDNNRLVQHQKMHTVKTPYECQECGERFICGSTLKCHESVHAREKQGFFVSGKILDQNPEQKEKCFKCNKCEKTFSCSKYLTQHERIHTRGVKPFECDQCGKAFGQSTRLIHHQRIHSRVRLYKWGEQGKAISSASLIKLQSFHTKEHPFKCNECGKTFSHSAHLSKHQLIHAGENPFKCSKCDRVFTQRNYLVQHERTHARKKPLVCNECGKTFRQSSCLSKHQRIHSGEKPYVCDYCGKAFGLSAELVRHQRIHTGEKPYVCQECGKAFTQSSCLSIHRRVHTGEKPYRCGECGKAFAQKANLTQHQRIHTGEKPYSCNVCGKAFVLSAHLNQHLRVHTQETLYQCQRCQKAFRCHSSLSRHQRVHNKQQYCL

SEQ ID NO: 388 - protein, 433 aa, synthetic construct
  REGION 1..433: note = ANM2 - Human, Full-length (AA)
MATSGDCPRSESQGEEPAECSEAGLLQEGVQPEEFVAIADYAATDETQLSFLRGEKILILRQTTADWWWGERAGCCGYIPANHVGKHVDEYDPEDTWQDEEYFGSYGTLKLHLEMLADQPRTTKYHSVILQNKESLTDKVILDVGCGTGIISLFCAHYARPRAVYAVEASEMAQHTGQLVLQNGFADIITVYQQKVEDVVLPEKVDVLVSEWMGTCLLFEFMIESILYARDAWLKEDGVIWPTMAALHLVPCSADKDYRSKVLFWDNAYEFNLSALKSLAVKEFFSKPKYNHILKPEDCLSEPCTILQLDMRTVQISDLETLRGELRFDIRKAGTLHGFTAWFSVHFQSLQEGQPPQVLSTGPFHPTTHWKQTLFMMDDPVPVHTGDVVTGSVVLQRNPVWRRHMSVALSWAVTSRQDPTSQKVGEKVFPIWR

SEQ ID NO: 389 - protein, 1113 aa, synthetic construct
  REGION 1..1113: note = KIBRA - Human, Full-length (AA)
MPRPELPLPEGWEEARDFDGKVYYIDHTNRTTSWIDPRDRYTKPLTFADCISDELPLGWEEAYDPQVGDYFIDHNTKTTQIEDPRVQWRREQEHMLKDYLVVAQEALSAQKEIYQVKQQRLELAQQEYQQLHAVWEHKLGSQVSLVSGSSSSSKYDPEILKAEIATAKSRVNKLKREMVHLQHELQFKERGFQTLKKIDKKMSDAQGSYKLDEAQAVLRETKAIKKAITCGEKEKQDLIKSLAMLKDGFRTDRGSHSDLWSSSSSLESSSFPLPKQYLDVSSQTDISGSFGINSNNQLAEKVRLRLRYEEAKRRIANLKIQLAKLDSEAWPGVLDSERDRLILINEKEELLKEMRFISPRKWTQGEVEQLEMARKRLEKDLQAARDTQSKALTERLKLNSKRNQLVRELEEATRQVATLHSQLKSLSSSMQSLSSGSSPGSLTSSRGSLVASSLDSSTSASFTDLYYDPFEQLDSELQSKVEFLLLEGATGFRPSGCITTIHEDEVAKTQKAEGGGRLQALRSLSGTPKSMTSLSPRSSLSSPSPPCSPLMADPLLAGDAFLNSLEFEDPELSATLCELSLGNSAQERYRLEEPGTEGKQLGQAVNTAQGCGLKVACVSAAVSDESVAGDSGVYEASVQRLGASEAAAFDSDESEAVGATRIQIALKYDEKNKQFAILIIQLSNLSALLQQQDQKVNIRVAVLPCSESTTCLFRTRPLDASDTLVFNEVFWVSMSYPALHQKTLRVDVCTTDRSHLEECLGGAQISLAEVCRSGERSTRWYNLLSYKYLKKQSRELKPVGVMAPASGPASTDAVSALLEQTAVELEKRQEGRSSTQTLEDSWRYEETSENEAVAEEEEEEVEEEEGEEDVFTEKASPDMDGYPALKVDKETNTETPAPSPTVVRPKDRRVGTPSQGPFLRGSTIIRSKTFSPGPQSQYVCRLNRSDSDSSTLSKKPPFVRNSLERRSVRMKRPSSVKSLRSERLIRTSLDLELDLQATRTWHSQLTQEISVLKELKEQLEQAKSHGEKELPQWLREDERFRLLLRMLEKRQMDRAEHKGELQTDKMMRAAAKDVHRLRGQSCKEPPEVQSFREKMAFFTRPRMNIPALSADDV

SEQ ID NO: 390 - protein, 2471 aa, synthetic construct
  REGION 1..2471: note = NOTCH2 (NOTC2) - Human, Full-length, alternative isoform (AA)
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 391 - protein, 745 aa, synthetic construct
  REGION 1..745: note = IKKA - Human, Full-length (AA)
MERPPGLRPGAGGPWEMRERLGTGGFGNVCLYQHRELDLKIAIKSCRLELSTKNRERWCHEIQIMKKLNHANVVKACDVPEELNILIHDVPLLAMEYCSGGDLRKLLNKPENCCGLKESQILSLLSDIGSGIRYLHENKIIHRDLKPENIVLQDVGGKIIHKIIDLGYAKDVDQGSLCTSFVGTLQYLAPELFENKPYTATVDYWSFGTMVFECIAGYRPFLHHLQPFTWHEKIKKKDPKCIFACEEMSGEVRFSSHLPQPNSLCSLVVEPMENWLQLMLNWDPQQRGGPVDLTLKQPRCFVLMDHILNLKIVHILNMTSAKIISFLLPPDESLHSLQSRIERETGINTGSQELLSETGISLDPRKPASQCVLDGVRGCDSYMVYLFDKSKTVYEGPFASRSLSDCVNYIVQDSKIQLPIIQLRKVWAEAVHYVSGLKEDYSRLFQGQRAAMLSLLRYNANLTKMKNTLISASQQLKAKLEFFHKSIQLDLERYSEQMTYGISSEKMLKAWKEMEEKAIHYAEVGVIGYLEDQIMSLHAEIMELQKSPYGRRQGDLMESLEQRAIDLYKQLKHRPSDHSYSDSTEMVKIIVHTVQSQDRVLKELFGHLSKLLGCKQKIIDLLPKVEVALSNIKEADNTVMFMQGKRQKEIWHLLKIACTQSSARSLVGSSLEGAVTPQTSAWLPPTSAEHDHSLSCVVTPQDGETSAQMIEENLNCLGHLSTIIHEANEEQGNSMMNLDWSWLTE

SEQ ID NO: 392 - protein, 710 aa, synthetic construct
  REGION 1..710: note = APBB1 - Human, Full-length (AA)
MSVPSSLSQSAINANSHGGPALSLPLPLHAAHNQLLNAKLQATAVGPKDLRSAMGEGGGPEPGPANAKWLKEGQNQLRRAATAHRDQNRNVTLTLAEEASQEPEMAPLGPKGLIHLYSELELSAHNAANRGLRGPGLIISTQEQGPDEGEEKAAGEAEEEEEDDDDEEEEEDLSSPPGLPEPLESVEAPPRPQALTDGPREHSKSASLLFGMRNSAASDEDSSWATLSQGSPSYGSPEDTDSFWNPNAFETDSDLPAGWMRVQDTSGTYYWHIPTGTTQWEPPGRASPSQGSSPQEESQLTWTGFAHGEGFEDGEFWKDEPSDEAPMELGLKEPEEGTLTFPAQSLSPEPLPQEEEKLPPRNTNPGIKCFAVRSLGWVEMTEEELAPGRSSVAVNNCIRQLSYHKNNLHDPMSGGWGEGKDLLLQLEDETLKLVEPQSQALLHAQPIISIRVWGVGRDSGRERDFAYVARDKLTQMLKCHVFRCEAPAKNIATSLHEICSKIMAERRNARCLVNGLSLDHSKLVDVPFQVEFPAPKNELVQKFQVYYLGNVPVAKPVGVDVINGALESVLSSSSREQWTPSHVSVAPATLTILHQQTEAVLGECRVRFLSFLAVGRDVHTFAFIMAAGPASFCCHMFWCEPNAASLSEAVQAACMLRYQKCLDARSQASTSCLPAPPAESVARRVGWTVRRGVQSLWGSLKPKRLGAHTP

SEQ ID NO: 393 - protein, 294 aa, synthetic construct
  REGION 1..294: note = SMN2 - Human, Full-length (AA)
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 394 - protein, 314 aa, synthetic construct
  REGION 1..314: note = SERTAD2 - Human, Full-length (AA)
MLGKGGKRKFDEHEDGLEGKIVSPCDGPSKVSYTLQRQTIFNISLMKLYNHRPLTEPSLQKTVLINNMLRRIQEELKQEGSLRPMFTPSSQPTTEPSDSYREAPPAFSHLASPSSHPCDLGSTTPLEACLTPASLLEDDDDTFCTSQAMQPTAPTKLSPPALLPEKDSFSSALDEIEELCPTSTSTEAATAATDSVKGTSSEAGTQKLDGPQESRADDSKLMDSLPGNFEITTSTGFLTDLTLDDILFADIDTSMYDFDPCTSSSGTASKMAPVSADDLLKTLAPYSSQPVTPSQPFKMDLTELDHIMEVLVGS

SEQ ID NO: 395 - protein, 752 aa, synthetic construct
  REGION 1..752: note = MYBA - Human, Full-length (AA)
MAKRSRSEDEDDDLQYADHDYEVPQQKGLKKLWNRVKWTRDEDDKLKKLVEQHGTDDWTLIASHLQNRSDFQCQHRWQKVLNPELIKGPWTKEEDQRVIELVQKYGPKRWSLIAKHLKGRIGKQCRERWHNHLNPEVKKSSWTEEEDRIIYEAHKRLGNRWAEIAKLLPGRTDNSIKNHWNSTMRRKVEQEGYLQDGIKSERSSSKLQHKPCAAMDHMQTQNQFYIPVQIPGYQYVSPEGNCIEHVQPTSAFIQQPFIDEDPDKEKKIKELEMLLMSAENEVRRKRIPSQPGSFSSWSGSFLMDDNMSNTLNSLDEHTSEFYSMDENQPVSAQQNSPTKFLAVEANAVLSSLQTIPEFAETLELIESDPVAWSDVTSFDISDAAASPIKSTPVKLMRIQHNEGAMECQFNVSLVLEGKKNTCNGGNSEAVPLTSPNIAKFSTPPAILRKKRKMRVGHSPGSELRDGSLNDGGNMALKHTPLKTLPFSPSQFFNTCPGNEQLNIENPSFTSTPICGQKALITTPLHKETTPKDQKENVGFRTPTIRRSILGTTPRTPTPFKNALAAQEKKYGPLKIVSQPLAFLEEDIREVLKEETGTDLFLKEEDEPAYKSCKQENTASGKKVRKSLVLDNWEKEESGTQLLTEDISDMQSENRFTTSLLMIPLLEIHDNRCNLIPEKQDINSTNKTYTLTKKKPNPNTSKVVKLEKNLQSNCEWETVVYGKTEDQLIMTEQARRYLSTYTATSSTSRALIL